(19) United States
(12) Patent Application Publication
(10) Pub. No.: US 2026/0055108 A1
(43) Pub. Date: Feb. 26, 2026

(54) TRIAZINE COMPOUNDS AND USES THEREOF

(71) Applicant: HUTCHMED LIMITED, Shanghai (CN)

(72) Inventors: Weihan ZHANG, Shanghai (CN); Haibin YANG, Shanghai (CN); Huaqing CAI, Shanghai (CN); Wei-Guo SU, Shanghai (CN)

(21) Appl. No.: 19/108,036

(22) PCT Filed: Sep. 1, 2023
(86) PCT No.: PCT/CN2023/116442

§ 371 Date: Feb. 28, 2025

(30) Foreign Application Priority Data
Sep. 2, 2022 (CN) 202211074820.6
Aug. 18, 2023 (CN) 202311048952.6

(51) Int. Cl. C07D 471/10; A61K 31/53; A61K 31/5377; A61K 45/06; A61P 35/02; C07B 59/00; C07D 401/14; C07D 403/14; C07D 519/00

(52) U.S. Cl. CPC C07D 471/10; A61K 31/53; A61K 31/5377; A61K 45/06; A61P 35/02; C07B 59/002; C07D 401/14; C07D 403/14; C07D 519/00

(57) ABSTRACT

Provided are a triazine compound of formula (I), a pharmaceutical composition comprising same, a preparation method therefor and the use thereof.

### TECHNICAL FIELD

The present invention relates to a triazine compound, a pharmaceutical composition comprising same, a preparation method therefor and the use thereof.

### BACKGROUND ART

The occurrence of acute leukemia is related to genetic heterogeneity and epigenetic abnormal changes (Rodriguez-Paredes M, et al. Nat Med. 2011; 17(3): 330-339), which poses a huge challenge to the treatment of acute leukemia. MLL (Mixed lineage leukemia) protein, also referred to as MLL1 or KMT2A, is a protein expressed in hematopoietic cells and necessary for the normal development of hematopoietic cells. It consists of two subunits, the N-terminal part and the C-terminal part formed by the cleavage of MLL protein precursor. MLL protein comprises two main functional domains, such as the C-terminal SET (Su(var)3-9, Enhancer-of-zeste and Trithorax) domain with histone H3 lysine 4 (H3K4) methyltransferase activity and the N-terminal DNA binding domain (AT hook sequence). MLL protein usually forms a complex with a chaperonin to regulate gene expression through epigenetic mechanisms (Krivtsov A V, et al. Nat Rev Cancer. 2007; 7: 823-33).

Studies have found that MLL gene translocation occurs in approximately 5%-10% of patients with acute leukemia (including AML and ALL), and is particularly prevalent in infantile leukemia, accounting for approximately 80% of acute lymphocytic leukemia (ALL) cases in infants and young children, which cases show strong resistance to chemotherapy (Teachey D T, et al. Br J Haematol. 2013; 162(5): 606-620). MLL translocation may result in the fusion of the MLL N-terminal fragment to more than 80 chaperonins (Slany R K, et al. Haematologica. 2009; 94(7): 984-993). Through genome-wide gene expression analysis of samples in MLL fusion leukemia and MLL wild-type leukemia, it has been found that their gene expression characteristics are significantly different. The most frequently overexpressed genes in MLL fusion leukemia are HOX (Homeobox) cluster genes (particularly HOXA7-HOXA10) and MEIS1, a cofactor of HOX genes. HOX family genes encode transcription factors, thereby controlling developmental processes, especially the development of the hematopoietic system (Li Z, et al. Cancer Res (2009) 69: 1109-16). In the hematopoietic system, HOX and MEIS1 genes are highly expressed in both stem cells and early lineage progenitor cells, and the expression levels decrease with differentiation. Persistent expression of MEIS1 and HOX genes has been observed in a variety of leukemias, including MLL fusion leukemia (Kawagoe H, et al. Leukemia (1999) 13: 687-98). Therefore, dysregulated expression of the HOX developmental regulator and its cofactor MEIS1 by MLL fusion proteins plays a key role in the stem cell-like characteristics of MLL fusion leukemia, and endows or maintains these cells with advantages in self-renewal properties, growth and survival, thereby promoting the oncogenic potential of the MLL fusion proteins (Winters A C, et al. Front Pediatr. 2017; 5: 4). In addition to gene fusions caused by translocation, MLL partial tandem duplications (PTDs) may also be found in 5%-10% of children and adults with ALL and AML. Epidemiological investigation shows that MLL-PTD indicates worse recurrence-free survival rate in patients with acute leukemia (Döhner K, et al. J Clin Oncol. 2002; 20: 3254-61). Therefore, there is a great medical need for leukemia caused by MLL abnormalities, and it is necessary to develop new treatment methods for the leukemia.

Studies have shown that the oncogenic function of MLL fusion proteins mainly depends on their direct interaction with menin proteins (Yokoyama A, et al. Cell. 2005; 123(2): 207-218). Menin is a 67 kDa protein encoded by MEN1 (Multiple Endocrine Neoplasia I) gene located on chromosome 11q13. Menin is widely expressed and mainly localized in the nucleus. Although menin lacks a defined biological functional domain, menin protein can bind to a variety of functional proteins, participate in the regulation of gene expression, and affect cell growth and development (Balogh K, et al. Trends Endocrinol Metab. 2006; 17(9): 357-364). As one of the most important binding molecules, menin protein can directly bind to the N-terminal menin binding motif (MBM) of MLL fusion protein, and recruits MLL or MLL fusion protein to target genes (including HOXA9 or MEIS1) (Grembecka J, et al. J. Biol. Chem. 2010; 285(52): 40690-40698).

Studies have shown that if MLL fusion protein loses its binding to menin protein, it will lose its oncogenic properties in vitro and in vivo (Caslini C, et al. Cancer. Res. 2007; 67(15): 7275-7283).

In addition, if the N-terminus of MLL-ENL fusion protein is mutated, the fusion protein will not bind to menin, thereby inhibiting HOX gene expression and the potential to induce leukemia in mice (Yokoyama A, et al. Cell. 2005). The MLL fusion protein/menin interaction can be inhibited by using small molecule inhibitors, which can significantly inhibit the proliferation of MLL fusion-related leukemia cells and promote the cell differentiation (Grembecka J, et al. Nat. Chem. Biol. 2012; 8(3): 277-284).

NPM1 is a widely expressed nucleophosmin that shuttles between the nucleus and the cytoplasm as a chaperone molecule, participates in the biosynthesis of ribosomes, and maintains genome stability (Heath E M, et al. Leukemia. 2017; 31(4): 798-807). Studies have shown that NPM1 mutation is found in 20%-30% of patients with AML (Papaemmanuil E, et al. N Engl J Med. 2016; 374: 2209-21). Moreover, studies have shown that the menin/MLL1 wild-type interaction also plays an important role in acute myelogenous leukemia with mutation in the NPM1 gene. After treatment with the first-generation menin-MLL1 inhibitor, AML cells with NPM1 mutation undergo growth arrest and differentiation (Uckelmann H J, et al. Science. 2020; 367: 586-90). These results further support that more potent menin-MLL1 inhibitors can be developed for leukemia patients with NPM1 mutation.

Currently, a plurality of small molecule menin inhibitors against acute leukemia are being explored in pre-clinical or clinical trials. However, the small molecule menin inhibitors currently in the clinical stage still have great defects, such as to-be-improved menin inhibitory activity, poor compound stability and poor pharmacokinetic characteristics, making it difficult for the compound exposure to reach a clinically effective dose. Therefore, it is of great clinical significance to develop a new generation of more potent small molecule menin inhibitors with good pharmacokinetic properties and less toxic and side effects.

### SUMMARY OF THE INVENTION

The present invention addresses the aforementioned needs in the art. The present invention provides an inhibitor compound with a new structure having a menin inhibitory activity. Compared with existing inhibitors in the prior art, the compounds of the present invention have comparable or enhanced menin inhibitory activity and good pharmacokinetic properties due to their improved structural patterns, so that they can be administered in a convenient manner and are absorbed more easily in vivo, with less toxic and side effects.

In this way, the present invention provides a compound of formula (I):

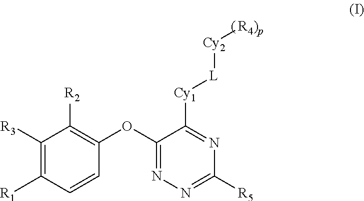

or a pharmaceutically acceptable salt, a solvate, a racemic mixture, an enantiomer, a diastereomer or a tautomer thereof, or a deuterate thereof, wherein
R₁ is selected from hydrogen, halogen, —CN, —OH, —NH₂, C₁₋₆ alkyl, C₁₋₆ haloalkyl, —O—(C₁₋₆ alkyl) and —O—(C₁₋₆ haloalkyl);
R₂ is selected from hydrogen, halogen, —CN, —OH, —NH₂, C₁₋₆ alkyl, C₁₋₆ haloalkyl, —(C₁₋₆ alkyl)-O—(C₁₋₆ alkyl), —(C₁₋₆ alkyl)-OH, —(C₁₋₆ alkyl)-CN, —O—(C₁₋₆ alkyl), —O—(C₁₋₆ haloalkyl), —O—(C₃₋₈ cycloalkyl), —O-(4-8 membered heterocyclyl), C₃₋₈ cycloalkyl, 4-8 membered heterocyclyl, phenyl, 5-12 membered heteroaryl, —S—(C₁₋₆ alkyl), —S—(C₃₋₈ cycloalkyl), —S-(4-8 membered heterocyclyl), —NH(C₁₋₆ alkyl), —N(C₁₋₆ alkyl)₂, —NHCONH₂, —NHCO(C₁₋₆ alkyl), —CONRₐR_{b}, —CSNRₐR_{b}, —COR_{c} and —COORₑ, wherein the C₃₋₈ cycloalkyl, 4-8 membered heterocyclyl, phenyl and 5-12 membered heteroaryl are each optionally substituted with one or more groups independently selected from halogen, —OH, oxo, —CN, —NH₂, —CONH₂, C₁₋₆ alkyl, C₂₋₆ alkenyl, C₂₋₆ alkynyl, C₁₋₆ haloalkyl, —(C₁₋₆ alkyl)-O—(C₁₋₆ alkyl), —(C₁₋₆ alkyl)-OH, —(C₁₋₆ alkyl)-CN, —O—(C₁₋₆ alkyl), —O—(C₁₋₆ haloalkyl), —S—(C₁₋₆ alkyl), —NH(C₁₋₆ alkyl), —N(C₁₋₆ alkyl)₂, —CONH(C₁₋₆ alkyl) and —CON(C₁₋₆ alkyl)₂;
R₃ is selected from hydrogen, halogen, —CN, —OH, —NH₂, C₁₋₆ alkyl, C₁₋₆ haloalkyl, —O—(C₁₋₆ alkyl) and —O—(C₁₋₆ haloalkyl);
or R₂ and R₃ together with the carbon atom to which they are attached form 5-6 membered heteroaryl or 4-6 membered heterocyclyl, wherein the 5-6 membered heteroaryl and 4-6 membered heterocyclyl are each optionally substituted with one or more groups independently selected from halogen, —OH, oxo, —CN, —NH₂, —CONH₂, C₁₋₆ alkyl, C₂₋₆ alkenyl, C₂₋₆ alkynyl, C₁₋₆ haloalkyl, —(C₁₋₆ alkyl)-O—(C₁₋₆ alkyl), —(C₁₋₆ alkyl)-OH, —(C₁₋₆ alkyl)-CN, —O—(C₁₋₆ alkyl) and —O—(C₁₋₆ haloalkyl);
R₅ is selected from hydrogen, halogen, —CN, —OH, C₁₋₆ alkyl, C₁₋₆ haloalkyl, —(C₁₋₆ alkyl)-O—(C₁₋₆ alkyl), —(C₁₋₆ alkyl)-OH, —(C₁₋₆ alkyl)-CN, —O—(C₁₋₆ alkyl), —O—(C₁₋₆ haloalkyl), C₃₋₈ cycloalkyl, 4-8 membered heterocyclyl, —S—(C₁₋₆ alkyl), —NHCONH₂, —NHCO(C₁₋₆ alkyl) and —NRₐR_{b};
Cy₁ is 4-12 membered heterocyclyl, which is optionally substituted with one or more groups independently selected from halogen, —OH, oxo, —CN, —NH₂, —CONH₂, C₁₋₆ alkyl, C₂₋₆ alkenyl, C₂₋₆ alkynyl, C₁₋₆ haloalkyl, —(C₁₋₆ alkyl)-O—(C₁₋₆ alkyl), —(C₁₋₆ alkyl)-OH, —(C₁₋₆ alkyl)-CN, —O—(C₁₋₆ alkyl) and —O—(C₁₋₆ haloalkyl);
Cy₂ is selected from C₃₋₈ cycloalkyl, 4-9 membered heterocyclyl, aryl and 5-14 membered heteroaryl;
R₄ is independently selected from halogen, —CN, —OH, oxo, —SH, C₁₋₆ alkyl, C₂₋₆ alkenyl, C₂₋₆ alkynyl, —O—(C₁₋₆ alkyl), —O—(C₁₋₆ haloalkyl), —O—(C₃₋₈ cycloalkyl), —O-(4-8 membered heterocyclyl), —(C₁₋₆ alkyl)ₘ-(C₃₋₈ cycloalkyl), —(C₁₋₆ alkyl)ₘ-(4-8 membered heterocyclyl), —(C₁₋₆ alkyl)ₘ-phenyl, —(C₁₋₆ alkyl)ₘ-(5-12 membered heteroaryl), —S—(C₁₋₆ alkyl), —S—(C₃₋₈ cycloalkyl), —S-(4-8 membered heterocyclyl), —NHCONH₂, —CONRₐR_{b}, —COR_{c}, —COOR_{c}, —NRₐR_{b}, —NR_{d}COR_{c}, —NR_{d}S(O)ₙR_{f}, —S(O)ₙR_{f} and —S(O)ₙNRₐR_{b}, wherein the C₁₋₆ alkyl, C₃₋₈ cycloalkyl, 4-8 membered heterocyclyl, phenyl and 5-12 membered heteroaryl are each optionally substituted with one or more groups independently selected from halogen, —OH, oxo, —SH, —CN, —NH₂, —CONH₂, C₁₋₆ alkyl, C₂₋₆ alkenyl, C₂₋₆ alkynyl, C₁₋₆ haloalkyl, —(C₁₋₆ alkyl)-O—(C₁₋₆ alkyl), —(C₁₋₆ alkyl)-OH, —(C₁₋₆ alkyl)-CN, —O—(C₁₋₆ alkyl), —O—(C₁₋₆ haloalkyl), —O—(C₃₋₈ cycloalkyl), —O-(4-8 membered heterocyclyl), —S—(C₁₋₆ alkyl), —NH(C₁₋₆ alkyl), —N(C₁₋₆ alkyl)₂, —CO(C₁₋₆ alkyl), —CO(C₂₋₆ alkenyl), —CO(C₂₋₆ alkynyl), —CONH(C₁₋₆ alkyl), —CON(C₁₋₆ alkyl)₂, C₃₋₈ cycloalkyl and 4-8 membered heterocyclyl;
L is absent, or L is CH₂;
Rₐ, R_{b}, R_{c}, R_{d} and Rₑ are each independently selected from hydrogen, C₁₋₆ alkyl, C₂₋₆ alkenyl, C₂₋₆ alkynyl, —(C₁₋₆ alkyl)ₘ-(C₃₋₈ cycloalkyl), —(C₁₋₆ alkyl)ₘ-(4-8 membered heterocyclyl), —(C₁₋₆ alkyl)ₘ-phenyl and —(C₁₋₆ alkyl)ₘ-(5-12 membered heteroaryl), wherein the C₁₋₆ alkyl, C₃₋₈ cycloalkyl, 4-8 membered heterocyclyl, phenyl and 5-12 membered heteroaryl are each optionally substituted with one or more groups independently selected from halogen, —OH, oxo, —SH, —CN, —NH₂, —CONH₂, C₁₋₆ alkyl, C₂₋₆ alkenyl, C₂₋₆ alkynyl, C₁₋₆ haloalkyl, —(C₁₋₆ alkyl)-O—(C₁₋₆ alkyl), —(C₁₋₆ alkyl)-OH, —(C₁₋₆ alkyl)-CN, —O—(C₁₋₆ alkyl), —O—(C₁₋₆ haloalkyl), —O—(C₃₋₈ cycloalkyl), —O-(4-8 membered heterocyclyl), —S—(C₁₋₆ alkyl), —NH(C₁₋₆ alkyl), —N(C₁₋₆ alkyl)₂, —NH(C₃₋₈ cycloalkyl), —NH(4-8 membered heterocyclyl), —CO(C₁₋₆ alkyl), —CO(C₂₋₆ alkenyl), —CO(C₂₋₆ alkynyl), —CO(C₃₋₈ cycloalkyl), —CO(4-8 membered heterocyclyl), —CONH(C₁₋₆ alkyl), —CONH(C₃₋₈ cycloalkyl), —CONH(4-8 membered heterocyclyl) and —CON(C₁₋₆ alkyl)₂;
R_{f} is —CH₃;
p is 0, 1, 2, 3, 4 or 5;
m is 0 or 1; and
n is 1 or 2;
provided that, R₄ is not —NH(6-membered nitrogen-containing heteroaryl) or —NH(phenyl substituted with F).

The above-mentioned compounds and the active compounds (including compounds of general formulas and specific compounds) disclosed in the context of the present invention, and pharmaceutically acceptable salts thereof, or solvates, racemic mixtures, enantiomers, diastereomers or tautomers thereof, or deuterates thereof are collectively referred to herein as “compounds of the present invention”.

The present invention also provides a pharmaceutical composition, comprising the compounds of the present invention, and optionally comprising a pharmaceutically acceptable excipient.

The present invention also provides a method of in vivo or in vitro inhibiting menin-MLL interaction, comprising contacting menin with an effective amount of the compounds of the present invention.

The present invention also provides a method of treating or preventing a disease mediated by menin-MLL interaction or at least in part by menin-MLL interaction, comprising administering to a subject in need thereof an effective amount of the compounds of the present invention.

The present invention also provides a method of treating or preventing cancer, comprising administering to a subject in need thereof an effective amount of the compounds of the present invention.

The present invention also provides the use of the compounds of the present invention in the treatment or prevention of a disease mediated by menin-MLL interaction or at least in part by menin-MLL interaction.

The present invention also provides the use of the compounds of the present invention in the treatment or prevention of cancer.

The present invention also provides the use of the compounds of the present invention in the manufacture of a medicament for treating or preventing a disease mediated by menin-MLL interaction or at least in part by menin-MLL interaction.

The present invention also provides the use of the compounds of the present invention in the manufacture of a medicament for treating or preventing cancer.

The present invention also provides the compounds of the present invention for in vivo or in vitro inhibiting menin-MLL interaction.

The present invention also provides the compounds of the present invention for use as a medicament.

The present invention also provides the compounds of the present invention for use as a medicament for treating or preventing a disease mediated by menin-MLL interaction or at least in part by menin-MLL interaction, especially for treating or preventing cancer.

The present invention also provides a pharmaceutical combination, comprising the compounds of the present invention and at least one additional therapeutic agent, wherein the additional therapeutic agent is preferably selected from: an anti-neoplastic active agent, an anti-inflammatory agent or an immunomodulator, wherein the anti-neoplastic active agent includes a chemotherapeutic agent, an immune checkpoint inhibitor or agonist, and a targeted therapeutic agent.

The present invention further provides a kit for treating or preventing a disease mediated by menin-MLL interaction or at least in part by menin-MLL interaction. The kit can comprise the pharmaceutical composition of the present invention and instructions for use, wherein the pharmaceutical composition comprises the compounds of the present invention.

In some embodiments of the present invention, the “disease mediated by menin-MLL interaction or at least in part by menin-MLL interaction” refers to cancer, such as a hematologic malignancy or solid tumor, including leukemia, lymphoma and myeloma, such as acute leukemia, chronic leukemia, myeloid leukemia, myelogenous leukemia, lymphocytic leukemia, lymphoblastic leukemia, acute myelogenous leukemia (AML), chronic myelogenous leukemia (CML), acute lymphocytic leukemia (ALL), B-cell acute lymphocytic leukemia (B-ALL), T-cell prolymphocytic leukemia (T-PLL), chronic lymphocytic leukemia (CLL), chronic myelocytic leukemia, large granular lymphocytic leukemia, hairy cell leukemia (HCL), mixed lineage leukemia (MLL), MLL-related leukemia, MLL-rearranged leukemia (MLL-r), MLL-PTD leukemia, MLL-positive leukemia, NPM1 mutant leukemia, leukemia exhibiting HOX/MEIS1 gene expression signatures, myelodysplastic syndrome (MDS), myeloproliferative neoplasm (MPN), multiple myeloma (MM), Hodgkin's lymphoma, non-Hodgkin's lymphoma, diffuse large B-cell lymphoma (DLBCL), B-cell lymphoma, T-cell lymphoma, mantle cell lymphoma, follicular lymphoma (FL), Waldenstrom's macroglobulinemia, prostate cancer, breast cancer, lung cancer, liver cancer, colon cancer, colorectal cancer, pancreatic cancer, melanoma, and glioblastoma (GBM).

### DETAILED DESCRIPTION OF THE INVENTION

### Definitions

As used in the present application, the following words, phrases and symbols are generally intended to have the meanings as set forth below, except to the extent that the context in which they are used indicates otherwise.

A dash (“-”) that is not between two letters or symbols is used to indicate a point of attachment for a substituent. For example, —O(C₁₋₆ alkyl) refers to the attachment of C₁₋₆ alkyl to the rest of the molecule through an oxygen atom.

The term “alkyl” as used herein refers to a straight or branched saturated hydrocarbon radical containing 1-18 carbon atoms (C₁₋₁₈), preferably 1-10 carbon atoms (C₁₋₁₀), more preferably 1-6 carbon atoms (C₁₋₆), and further more preferably 1-4 carbon atoms (C₁-4) or 1-3 carbon atoms (C₁₋₃). For example, “C₁₋₆ alkyl” refers to an alkyl containing 1-6 carbon atoms. “C₁₋₃ alkyl” refers to an alkyl containing 1-3 carbon atoms. Examples of C₁₋₆ alkyl include, but are not limited to, methyl, ethyl, propyl (e.g. n-propyl, i-propyl), butyl (e.g. n-butyl, i-butyl, s-butyl and t-butyl), pentyl (e.g. n-pentyl, i-pentyl, neo-pentyl), hexyl, and the like. When used as a linker (e.g., in the definition of L) or between two dashes (“-”) (e.g., —(C₁₋₆ alkyl)-OH), the alkyl refers to an alkylene.

The term “alkenyl” as used herein refers to a straight or branched unsaturated hydrocarbon radical containing one or more, for example 1, 2, or 3 carbon-carbon double bonds (C═C) and 2-18 carbon atoms (C₂₋₁₈), preferably 2-10 carbon atoms (C₂₋₁₀), more preferably 2-6 carbon atoms (C₂₋₆), and further more preferably 2-4 carbon atoms (C₂₋₄). For example, “C₂₋₆ alkenyl” refers to an alkenyl containing 2-6 carbon atoms. “C₂₋₄ alkenyl” refers to an alkenyl containing 2-4 carbon atoms. Examples of C₂₋₆ alkenyl include, but are not limited to, vinyl, propenyl (e.g. 2-propenyl), and butenyl (e.g. 2-butenyl), and the like. The point of attachment for the alkenyl can be on or not on the double bond carbon.

The term “alkynyl” as used herein refers to a straight or branched unsaturated hydrocarbon radical containing one or more, for example 1, 2, or 3, carbon-carbon triple bonds (C≡C) and 2-18 carbon atoms (C₂₋₁₈), preferably 2-10 carbon atoms (C₂₋₁₀), more preferably 2-6 carbon atoms (C₂₋₆), and further more preferably 2-4 carbon atoms (C₂₋₄). For example, “C₂₋₆ alkynyl” refers to an alkynyl containing 2-6 carbon atoms. “C₂₋₄ alkynyl” refers to an alkynyl containing 2-4 carbon atoms. Examples of C₂₋₆ alkynyl include, but are not limited to, ethynyl, propynyl (e.g. 2-propynyl), and butynyl (e.g. 2-butynyl), and the like. The point of attachment for the alkynyl can be on or not on the triple bond carbon.

The term “halogen” or “halo” as used herein means fluoro, chloro, bromo, and iodo, preferably fluoro, chloro and bromo, more preferably fluoro and chloro.

The term “haloalkyl” as used herein refers to an alkyl radical, as defined herein, in which one or more, for example 1, 2, 3, 4, or 5, or all hydrogen atoms are replaced with halogen atoms, and when more than one hydrogen atoms are replaced with halogen atoms, the halogen atoms may be the same or different from each other. For example, “C₁₋₆ haloalkyl” refers to a haloalkyl as defined herein containing 1-6 carbon atoms. “C₁₋₄ haloalkyl” refers to a haloalkyl as defined herein containing 1-4 carbon atoms. Examples of C₁₋₆ haloalkyl include, but are not limited to —CF₃, —CHF₂, —CH₂F, —CH₂CF₃, —CH(CF₃)₂, and the like.

The term “cycloalkyl” as used herein refers to saturated or partially unsaturated cyclic hydrocarbon radical having 3-12 ring carbon atoms (C₃₋₁₂), such as 3-8 ring carbon atoms (C₃₋₈), 5-7 ring carbon atoms (C₅₋₇), 4-7 ring carbon atoms (C₄₋₇) or 3-6 ring carbon atoms (C₃₋₆), which may have one or more rings, such as 1, 2, or 3 rings, preferably 1 or 2 rings. For example, “C₃₋₈ cycloalkyl” or “3-8 membered cycloalkyl” refers to a cycloalkyl containing 3-8 ring carbon atoms; “C₃₋₆ cycloalkyl” or “3-6 membered cycloalkyl” refers to a cycloalkyl containing 3-6 ring carbon atoms. The cycloalkyl may include a fused or bridged ring, or a spirocyclic ring. The rings of the cycloalkyl may be saturated or have one or more, for example, one or two double bonds (i.e. partially unsaturated), but not fully conjugated, and not an aryl as defined herein. Examples of cycloalkyl include, but are not limited to cyclopropyl, cyclobutyl, cyclopentyl, cyclohexyl, spiro[2.2]pentyl, spiro[3.3]heptyl, bicyclo[3.1.0]hexyl, cyclopropenyl, cyclobutenyl, cyclopentenyl, cyclopentadienyl, cyclohexenyl, etc.

The term “heterocyclyl” or “heterocycle” as used herein can be used interchangeably and each refers to saturated or partially unsaturated cyclic radicals having 3-12 ring atoms, such as 4-12 ring atoms (4-12 membered heterocyclyl), 3-8 ring atoms (3-8 membered heterocyclyl), 4-9 ring atoms (4-9 membered heterocyclyl), 4-8 ring atoms (4-8 membered heterocyclyl), 4-6 ring atoms (4-6 membered heterocyclyl) or 4-5 ring atoms (4-5 membered heterocyclyl), and containing one or more, for example 1, 2 or 3, preferably 1 or 2 heteroatoms independently selected from N, O and S in the rings, with the remaining ring atoms being carbon; it may have one or more rings, for example 1, 2 or 3, preferably 1 or 2 rings. The heterocyclyl also includes those wherein the N or S heteroatom are optionally oxidized to various oxidation states. The point of attachment of heterocyclyl can be on the N heteroatom or carbon. For example, “4-9 membered heterocyclyl or 4-9 membered heterocycle” represents a heterocyclyl having 4-9 (4, 5, 6, 7, 8 or 9) ring atoms comprising at least one, such as 1, 2 or 3, preferably 1 or 2 heteroatoms independently selected from N, O and S; “4-8 membered heterocyclyl or 4-8 membered heterocycle” represents a heterocyclyl having 4-8 (4, 5, 6, 7 or 8) ring atoms comprising at least one, such as 1, 2 or 3, preferably 1 or 2 heteroatoms independently selected from N, O and S; and “4-6 membered heterocyclyl or 4-6 membered heterocycle” represents a heterocyclyl having 4-6 (4, 5 or 6) ring atoms comprising at least one, preferably 1 or 2 heteroatoms independently selected from N, O and S (preferably N and O), which is preferably a monocyclic ring. The heterocyclyl also includes a monocyclic ring, fused or bridged ring, or a spirocyclic ring. The rings of the heterocyclyl may be saturated or have one or more, for example, one or two double bonds (i.e. partially unsaturated), but not fully conjugated, and not a heteroaryl as defined herein. Examples of heterocyclyl include, but are not limited to: 4-12 membered heterocyclyl, 4-9 membered heterocyclyl, 4-8 membered heterocyclyl and 4-6 membered heterocyclyl, such as 4-12 membered monocyclic or fused or bridged heterocyclyl, such as oxetanyl, azetidinyl, pyrrolidyl, tetrahydrofuranyl, dioxolanyl, dioxanyl, tetrahydropyranyl, dihydropyranyl (such as 3,6-dihydro-2H-pyranyl and 3,4-dihydro-2H-pyranyl), morpholinyl, thiomorpholinyl, piperidyl, piperazinyl, tetrahydropyridyl, dihydropyridyl, dihydropyrimidyl, dihydropyridazinyl, pyrazolidinyl, diazaspiro[3.5]nonyl (such as 2,7-diazaspiro[3.5]nonyl), azaspiro[3.5]nonyl (such as 2-azaspiro[3.5]nonyl), diazaspiro[3.4]octyl (such as 2,6-diazaspiro[3.4]octyl), and oxaspiro[3.3]heptyl.

The term “aryl” or “aromatic ring” as used herein can be used interchangeably and each refers to carbocyclic hydrocarbon radical of 6 to 14 carbon atoms, preferably 6 to 10 carbon atoms, consisting of one ring or more, such as two fused rings, wherein at least one ring is an aromatic ring. Examples of aryl include, but are not limited to phenyl, naphthalenyl, 1,2,3,4-tetrahydronaphthalenyl, phenanthryl, indenyl, indanyl, azulenyl, preferably phenyl and naphthalenyl, more preferably phenyl.

The term “heteroaryl” or “heteroaromatic ring” as used herein can be used interchangeably and each refers to: mono-, bi-, or tri-ring system having 5-15 ring atoms, preferably 5-14 ring atoms, more preferably 5-12 ring atoms, further preferably 5-10 ring atoms, and most preferably 5-6 or 8-10 ring atoms, wherein at least one ring is 5- or 6-membered aromatic ring containing one or more, for example 1 to 4, heteroatoms independently selected from N, O, and S, wherein S and N may be optionally oxidized to various oxidation states. When the total number of S and O atoms in the heteroaryl group exceeds 1, said S and O heteroatoms are not adjacent to one another. Preferably, the heteroaryl is 5-12 membered heteroaryl. For example, the heteroaryl includes:
a 5-6 membered monocyclic heteroaryl, i.e., a monocyclic ring aromatic hydrocarbyl having 5 or 6 ring atoms, wherein the ring atoms include one or more, such as 1, 2 or 3 heteroatoms independently selected from N, O and S (preferably N), and the remaining ring atoms are carbon atoms, such as 5-membered or 6-membered heteroaryl, such as 5-membered or 6-membered nitrogen- or oxygen- or sulfur-containing heteroaryl, 5-membered or 6-membered nitrogen- and oxygen- and/or sulfur-containing heteroaryl, such as pyridyl, N-oxide pyridyl, pyridinonyl (i.e., oxopyridyl, such as 2-oxopyridyl), pyrazinyl, pyrimidyl, triazinyl (such as 1,2,4-triazinyl), pyridazinyl, pyridazinonyl (i.e., oxopyridazinyl, such as 3-oxopyridazinyl), pyrazolyl, imidazolyl, oxazolyl, isoxazolyl, oxadiazolyl, thiazolyl, isothiazolyl, thiadiazolyl, tetrazolyl, triazolyl (such as 1,2,4-triazolyl), thienyl, furanyl, pyranyl, pyrrolyl, and
a 8-10 membered bicyclic heteroaryl, i.e., a bicycle aromatic hydrocarbyl having 8, 9 or 10 ring atoms, preferably a 9-10 membered bicyclic heteroaryl, wherein the ring atoms include one or more, such as 1, 2, 3 or 4, preferably 1, 2 or 3 heteroatoms independently selected from N, O and S (preferably N), and the remaining ring atoms are carbon atoms, wherein at least one ring is an aromatic ring, such as 8-10 membered nitrogen- or oxygen- or sulfur-containing bicyclic heteroaryl, 8-10 membered nitrogen- and oxygen- and/or sulfur-containing bicyclic heteroaryl, such as benzodioxolyl, benzoxazolyl, benzoisoxazolyl, benzothienyl, benzothiazolyl, benzoisothiazolyl, benzofuranyl, indolyl, indazolyl, purinyl, quinolinyl, quinolinonyl (i.e., oxoquinolinyl, such as 2-oxoquinolinyl), isoquinolinyl, dihydroquinolinyl, quinazolinyl, quinoxalinyl, imidazopyrimidyl (such as imidazo[1,2-c]pyrimidyl), imidazopyrazinyl (such as imidazo[1,2-a]pyrazinyl), imidazopyridyl (such as imidazo[1,2-a]pyridyl), imidazopyridazinyl (such as imidazo[1,2-b]pyridazinyl), pyrrolopyrazinyl (such as pyrrolo[1,2-a]pyrazinyl), pyrrolopyridyl (such as 1H-pyrrolo[2,3-b]pyridyl), pyrrolopyrimidyl (such as pyrrolo[3,4-d]pyrimidyl), pyrazolopyrazinyl (such as pyrazolo[1,5-a]pyrazinyl), pyrazolopyridyl (such as 1H-pyrazolo[3,4-b]pyridyl), pyrazolopyrimidyl (such as pyrazolo[1,5-a]pyrimidyl), triazolopyrimidyl (such as [1,2,4]triazolo[4,3-c]pyrimidyl and [1,2,4]triazolo[1,5-c]pyrimidyl), triazolopyrazinyl (such as [1,2,4]triazolo[1,5-a]pyrazinyl), triazolopyridyl (such as [1,2,4]triazolo[4,3-a]pyridyl and [1,2,4]triazolo[1,5-a]pyridyl), tetrazolopyridyl (such as tetrazolo[1,5-a]pyridyl), dihydro-pyrimidopyridazinyl (such as 3,4-dihydro-2H-pyrimido[1,2-b]pyridazinyl), dihydro-[1,4]dioxinopyridyl (such as 2,3-dihydro-[1,4]dioxino[2,3-b]pyridyl), dihydro-[1,4]dioxinopyridazinyl (such as 6,7-dihydro-[1,4]dioxino[2,3-c]pyridazinyl).

The term “—OH” as used herein refers to hydroxyl radical.

The term “—CN” as used herein refers to cyano radical.

The term “oxo” as used herein refers to ═O.

The term “amino protecting group” as used herein, also called nitrogen protecting group, refers to groups that reversibly block or protect the amino and/or amide functional groups so that the reaction proceeds on other functional groups of the compound. Examples of the amino protecting group include, but are not limited to, Boc (tert-butoxycarbonyl), benzyl, Pmb (p-methoxybenzyl), alkanoyl, triphenylmethyl, benzoyl, succinyl, phthaloyl, Fmoc (9-fluorenylmethoxycarbonyl), Cbz (benzyloxycarbonyl), and the like, preferably Boc (tert-butoxycarbonyl), benzyl, Pmb (p-methoxybenzyl), and Cbz (benzyloxycarbonyl); more preferably Boc (tert-butoxycarbonyl).

The term “optional” or “optionally” as used herein means that the subsequently described event or circumstance may or may not occur, and the description includes instances wherein the event or circumstance occur and instances in which it does not occur. For example, “optionally substituted with one or more” includes unsubstituted and substituted with 1, 2, 3 or more substituents as described. It will be understood by those skilled in the art, with respect to any group containing one or more substituents, that such groups are not intended to introduce any substitution or substitution patterns that are sterically impractical, chemically incorrect, synthetically non-feasible and/or inherently unstable.

The term “substituted” or “substituted with . . . ”, as used herein, means that one or more (such as, 1, 2, 3 or 4) hydrogens on the designated atom or group are replaced with one or more (such as 1, 2, 3 or 4) substituents, preferably the substituents selected from the indicated group of substituents or radicals, provided that the designated atom's normal valence is not exceeded. The said substituents may be the same or different from each other. The term “substituted with one or more groups independently selected from . . . ” or “substituted with one or more . . . ” as used herein means that one or more hydrogens on the designated atom or group are independently replaced with one or more radicals from the indicated group of substituents or radicals, wherein the said radicals may be the same or different from each other. Preferably, “substituted with one or more groups independently selected from . . . ” or “substituted with one or more . . . ” means that the designated atom or group is substituted with 1, 2, 3, or 4 radicals independently selected from the indicated group of substituents or radicals, wherein the said radicals may be the same or different from each other. In some embodiments, when a substituent is oxo (i.e., ═O), then 2 hydrogens on a single atom are replaced by the oxo. An optional substituent can be any radicals, provided that combinations of substituents and/or variables result in a chemically correct and stable compound. A chemically correct and stable compound is meant to imply a compound that is sufficiently robust to survive sufficient isolation from a reaction mixture to be able to identify the chemical structure of the compound. Preferably, substituents are those exemplified in the compounds of the examples of the present application.

Unless otherwise specified, substituents are named into the core structure. For example, it is to be understood that when (cycloalkyl)alkyl is listed as a possible substituent, the point of attachment of this substituent to the core structure is in the alkyl portion.

It will be appreciated by the person of ordinary skill in the art (“POSITA”) that some of the compounds of formula (I) may contain one or more chiral centers and therefore exist in two or more stereoisomeric forms. The racemates of these isomers, the individual isomers and mixtures enriched in one enantiomer, as well as diastereomers when there are two chiral centers, and mixtures partially enriched with specific diastereomers are within the scope of the present invention. It will be further appreciated by the POSITA that the present invention includes all the individual stereoisomers (e.g. enantiomers, diastereomers), racemic mixtures or partially resolved mixtures of the compounds of formula (I) and, where appropriate, the individual tautomeric forms thereof.

The racemates can be used as such or can be resolved into their individual isomers. The resolution can afford stereochemically pure compounds or mixtures enriched in one or more isomers. Methods for separation of isomers are well known (cf. Allinger N. L. and Eliel E. L. in “Topics in Stereochemistry”, Vol. 6, Wiley Interscience, 1971) and include physical methods such as chromatography using a chiral adsorbent. Individual isomers can be prepared in chiral form from chiral precursors. Alternatively, individual isomers can be separated chemically from a mixture by: forming diastereomeric salts with a chiral acid (such as the individual enantiomers of 10-camphorsulfonic acid, camphoric acid, alpha-bromocamphoric acid, tartaric acid, diacetyltartaric acid, malic acid, pyrrolidone-5-carboxylic acid, and the like), fractionally crystallizing the salts, and then freeing one or both of the resolved bases, optionally repeating the process, so as obtain either or both substantially free of the other; i.e., in a form having an optical purity of >95%. Alternatively, the racemates can be covalently linked to a chiral compound (auxiliary) to produce diastereomers which can be separated by chromatography or by fractional crystallization after which time the chiral auxiliary is chemically removed to afford the pure enantiomers.

The term “tautomer” as used herein refers to constitutional isomers of compounds generated by rapid movement of an atom in two positions in a molecule. Tautomers readily interconvert into each other, e.g., enol form and ketone form are tipical tautomers.

A “pharmaceutically acceptable salt” is intended to mean a salt of a free acid or base of a compound of Formula (I) that is non-toxic, biologically tolerable, or otherwise biologically suitable for administration to the subject to be treated or prevented. For example, an acid addition salt includes such as a salt derived from an inorganic acid and an organic acid. For examples, see, generally, S. M. Berge, et al., “Pharmaceutical Salts”, J. Pharm. Sci., 1977, 66:1-19, and Handbook of Pharmaceutical Salts, Properties, Selection, and Use, Stahl and Wermuth, Eds., Wiley-VCH and VHCA, Zurich, 2002.

In addition, if a compound described herein is obtained as an acid addition salt, the free base can be obtained by basifying a solution of the acid addition salt. Conversely, if the product is a free base, an acid addition salt, particularly a pharmaceutically acceptable acid addition salt, may be produced by dissolving the free base in a suitable solvent and treating the solution with an acid, in accordance with conventional procedures for preparing acid addition salts from base compounds. The POSITA will recognize various synthetic methodologies that may be used without undue experimentation to prepare non-toxic pharmaceutically acceptable acid addition salts or base addition salts.

The term “solvate” means solvent addition forms that contain either stoichiometric or non-stoichiometric amounts of solvent. Some compounds have a tendency to trap a fixed molar ratio of solvent molecules in the solid state, thus forming a solvate. If the solvent is water, the solvate formed is a hydrate, when the solvent is alcohol, the solvate formed is an alcoholate. Hydrates are formed by the combination of one or more molecules of water, or less than one molecule of water, with one molecule of the substances in which the water retains its molecular state as H₂O, such combination being able to form one or more hydrates, for example, hemihydrate, monohydrate, and dihydrate.

The term “deuterate” means the compound formed by replacing one or more, for example, 1, 2, 3, 4, 5 or 6 hydrogen atoms in a compound with its isotope deuterium, wherein at the substitution position, the abundance of isotope deuterium (the deuteration degree) of the element deuterium is at least greater than the natural abundance. In some embodiments, the deuterate in the compound of formula (I) or in the compound of its sub-formula (I-1), (I-2), (I-3), (I-4) has a deuteration degree of at least 50% (e.g., 50%, 60%, 65%, 70%, 75%, 80%, 85%, 90%, 91%, 92%, 93%, 94%, 95%, 96%, 97%, 98%, 99%, 99.5%, 99.9%, or any value therebetween). In some embodiments, the compound of formula (I) or the compound of its sub-formula (I-1), (I-2), (I-3), (I-4) has a deuteration degree of greater than 99.9%, up to 100%.

As used herein, the terms “group(s)” and “radical(s)” are synonymous and are intended to indicate functional groups or fragments of molecules attachable to other fragments of molecules.

The term “active ingredient” is used to indicate a chemical substance which has biological activity. In some embodiments, an “active ingredient” is a chemical substance having pharmaceutical utility.

The term “pharmaceutical combination” as used herein means a product obtained by mixing or combining two or more active ingredients, including fixed and non-fixed combinations of active ingredients, such as a kit, and a pharmaceutical composition. The term “fixed combination” means that two or more active ingredients (such as compounds of the present invention and additional therapeutic agents) are administered simultaneously to a patient in the form of a single entity or dose. The term “non-fixed combination” means that two or more active ingredients (such as compounds of the present invention and additional therapeutic agents) are administered simultaneously, in parallel or successively to a patient in separate entities, wherein the administration provides the patient with a therapeutically effective level of the compound.

The terms “treating” or “treatment” or “prevention” of a disease or disorder, in the context of achieving therapeutic benefit, refer to administering one or more pharmaceutical substances, especially compounds of the present invention to a subject that has the disease or disorder, or has a symptom of a disease or disorder, or has a predisposition toward a disease or disorder, with the purpose to cure, heal, alleviate, relieve, alter, remedy, ameliorate, improve, or affect the disease or disorder, the symptoms of the disease or disorder, or the predisposition toward the disease or disorder. In some embodiments, the disease or disorder is cancer, such as hematologic malignancies or solid tumors, including leukemia, lymphoma and myeloma.

The terms “treating”, “contacting” and “reacting,” in the context of a chemical reaction, mean adding or mixing two or more reagents under appropriate conditions to produce the indicated and/or the desired product. It should be appreciated that the reaction which produces the indicated and/or the desired product may not necessarily result directly from the combination of two reagents which were initially added, i.e., there may be one or more intermediates which are produced in the mixture which ultimately lead to the formation of the indicated and/or the desired product.

The term “effective amount” as used herein refers to an amount or dose of a menin inhibitor sufficient to generally bring about a therapeutic benefit in patients in need of treatment or prevention for a disease or disorder mediated by menin-MLL interaction or at least in part by menin-MLL interaction. Effective amounts or doses of the active ingredient of the present disclosure may be ascertained by methods such as modeling, dose escalation studies or clinical trials, and by taking into consideration factors, e.g., the mode or route of administration or drug delivery, the pharmacokinetics of the agent, the severity and course of the disease or disorder, the subject's previous or ongoing therapy, the subject's health status and response to drugs, and the judgment of the attending physician.

An exemplary dose is in the range of from about 0.0001 to about 200 mg of active agent per kg of subject's body weight per day, such as from about 0.001 to 100 mg/kg/day, or about 0.01 to 35 mg/kg/day, or about 0.1 to 10 mg/kg daily in single or divided dosage units (e.g., BID, TID, QID). For a 70-kg human, an illustrative range for a suitable dosage amount is from about 0.05 to about 7 g/day, or about 0.2 to about 5 g/day. Once improvement of the patient's disease or disorder has occurred, the dose may be adjusted for maintenance treatment. For example, the dosage or the frequency of administration, or both, may be reduced as a function of the symptoms, to a level at which the desired therapeutic effect is maintained. Of course, if symptoms have been alleviated to an appropriate level, treatment may cease. Patients may, however, require intermittent treatment on a long-term basis upon any recurrence of symptoms.

The term “inhibition” or “inhibiting” indicates a decrease in the baseline activity of a biological activity or process. The term “inhibition of menin-MLL interaction” is a practical pharmaceutical activity for purposes of this disclosure and refers to a decrease in the menin-MLL interaction as a direct or indirect response to the presence of the compound of the present invention, relative to the menin-MLL interaction in the absence of the compound of the present invention. The decrease in interaction may be due to the direct interaction of the compound of the present invention with menin, or due to the interaction of the compound of the present invention, with one or more other factors that in turn affect the menin-MLL interaction. For example, the presence of the compound of the present invention may decrease the menin-MLL interaction by directly binding to the menin, by causing (directly or indirectly) another factor to decrease the menin-MLL interaction, or by (directly or indirectly) decreasing the amount of menin present in the cell or organism.

The term “subject” or “patient” as used herein means mammals and non-mammals. Mammals means any member of the mammalia class including, but not limited to, humans; non-human primates such as chimpanzees and other apes and monkey species; farm animals such as cattle, horses, sheep, goats, and swine; domestic animals such as rabbits, dogs, and cats; laboratory animals including rodents, such as rats, mice, and guinea pigs; and the like. Examples of non-mammals include, but are not limited to, birds, and the like. The term “subject” or “patient” does not denote a particular age or sex. In some embodiments, the subject or patient is a human.

In general, the term “about” is used herein to modify a numerical value above or below the stated value by a variance of 20%.

Technical and scientific terms used herein and not specifically defined have the meaning commonly understood by the POSITA to which the present disclosure pertains.

All numerical ranges herein shall be interpreted as disclosing each numerical value and subset of numerical values within the range, regardless of whether they are specifically otherwise disclosed. For example, when referring to any range of values, it should be regarded as referring to every value within the range of values, for example, every integer within the range of values. For example, C₁₋₆ as used herein represents the inclusion of 1, 2, 3, 4, 5 or 6 C. The invention relates to all values falling within the ranges, all smaller ranges and the upper or lower limits of the numerical range.

### DETAILED DESCRIPTION OF EMBODIMENTS

Embodiment 1. A compound of formula (I):

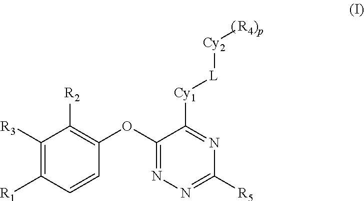

or a pharmaceutically acceptable salt, a solvate, a racemic mixture, an enantiomer, a diastereomer or a tautomer thereof, or a deuterate thereof, wherein
R₁ is selected from hydrogen, halogen, —CN, —OH, —NH₂, C₁₋₆ alkyl, C₁₋₆ haloalkyl, —O—(C₁₋₆ alkyl) and —O—(C₁₋₆ haloalkyl);
R₂ is selected from hydrogen, halogen, —CN, —OH, —NH₂, C₁₋₆ alkyl, C₁₋₆ haloalkyl, —(C₁₋₆ alkyl)-O—(C₁₋₆ alkyl), —(C₁₋₆ alkyl)-OH, —(C₁₋₆ alkyl)-CN, —O—(C₁₋₆ alkyl), —O—(C₁₋₆ haloalkyl), —O—(C₃₋₈ cycloalkyl), —O-(4-8 membered heterocyclyl), C₃₋₈ cycloalkyl, 4-8 membered heterocyclyl, phenyl, 5-12 membered heteroaryl, —S—(C₁₋₆ alkyl), —S—(C₃₋₈ cycloalkyl), —S-(4-8 membered heterocyclyl), —NH(C₁₋₆ alkyl), —N(C₁₋₆ alkyl)₂, —NHCONH₂, —NHCO(C₁₋₆ alkyl), —CONRₐR_{b}, —CSNRₐR_{b}, —COR_{c} and —COOR_{c}, wherein the C₃₋₈ cycloalkyl, 4-8 membered heterocyclyl, phenyl and 5-12 membered heteroaryl are each optionally substituted with one or more groups independently selected from halogen, —OH, oxo, —CN, —NH₂, —CONH₂, C₁₋₆ alkyl, C₂₋₆ alkenyl, C₂₋₆ alkynyl, C₁₋₆ haloalkyl, —(C₁₋₆ alkyl)-O—(C₁₋₆ alkyl), —(C₁₋₆ alkyl)-OH, —(C₁₋₆ alkyl)-CN, —O—(C₁₋₆ alkyl), —O—(C₁₋₆ haloalkyl), —S—(C₁₋₆ alkyl), —NH(C₁₋₆ alkyl), —N(C₁₋₆ alkyl)₂, —CONH(C₁₋₆ alkyl) and —CON(C₁₋₆ alkyl)₂;
R₃ is selected from hydrogen, halogen, —CN, —OH, —NH₂, C₁₋₆ alkyl, C₁₋₆ haloalkyl, —O—(C₁₋₆ alkyl) and —O—(C₁₋₆ haloalkyl);
or R₂ and R₃ together with the carbon atom to which they are attached form 5-6 membered heteroaryl or 4-6 membered heterocyclyl, wherein the 5-6 membered heteroaryl and 4-6 membered heterocyclyl are each optionally substituted with one or more groups independently selected from halogen, —OH, oxo, —CN, —NH₂, —CONH₂, C₁₋₆ alkyl, C₂₋₆ alkenyl, C₂₋₆ alkynyl, C₁₋₆ haloalkyl, —(C₁₋₆ alkyl)-O—(C₁₋₆ alkyl), —(C₁₋₆ alkyl)-OH, —(C₁₋₆ alkyl)-CN, —O—(C₁₋₆ alkyl) and —O—(C₁₋₆ haloalkyl);
R₅ is selected from hydrogen, halogen, —CN, —OH, C₁₋₆ alkyl, C₁₋₆ haloalkyl, —(C₁₋₆ alkyl)-O—(C₁₋₆ alkyl), —(C₁₋₆ alkyl)-OH, —(C₁₋₆ alkyl)-CN, —O—(C₁₋₆ alkyl), —O—(C₁₋₆ haloalkyl), C₃₋₈ cycloalkyl, 4-8 membered heterocyclyl, —S—(C₁₋₆ alkyl), —NHCONH₂, —NHCO(C₁₋₆ alkyl) and —NRₐR_{b};
Cy₁ is 4-12 membered heterocyclyl, which is optionally substituted with one or more groups independently selected from halogen, —OH, oxo, —CN, —NH₂, —CONH₂, C₁₋₆ alkyl, C₂₋₆ alkenyl, C₂₋₆ alkynyl, C₁₋₆ haloalkyl, —(C₁₋₆ alkyl)-O—(C₁₋₆ alkyl), —(C₁₋₆ alkyl)-OH, —(C₁₋₆ alkyl)-CN, —O—(C₁₋₆ alkyl) and —O—(C₁₋₆ haloalkyl);
Cy₂ is selected from C₃₋₈ cycloalkyl, 4-12 membered heterocyclyl, aryl and 5-14 membered heteroaryl;
R₄ is independently selected from halogen, —CN, —OH, oxo, —SH, C₁₋₆ alkyl, C₂₋₆ alkenyl, C₂₋₆ alkynyl, —O—(C₁₋₆ alkyl), —O—(C₁₋₆ haloalkyl), —O—(C₃₋₈ cycloalkyl), —O-(4-8 membered heterocyclyl), —(C₁₋₆ alkyl)ₘ-(C₃₋₈ cycloalkyl), —(C₁₋₆ alkyl)ₘ-(4-8 membered heterocyclyl), —(C₁₋₆ alkyl)ₘ-phenyl, —(C₁₋₆ alkyl)ₘ-(5-12 membered heteroaryl), —S—(C₁₋₆ alkyl), —S—(C₃₋₈ cycloalkyl), —S-(4-8 membered heterocyclyl), —NHCONH₂, —CONRₐR_{b}, —COR_{c}, —COORₑ, —NRₐR_{b}, —NR_{d}COR_{c}, —NR_{d}S(O)ₙR_{f}, —S(O)ₙR_{f} and —S(O)ₙNRₐR_{b}, wherein the C₁₋₆ alkyl, C₃₋₈ cycloalkyl, 4-8 membered heterocyclyl, phenyl and 5-12 membered heteroaryl are each optionally substituted with one or more groups independently selected from halogen, —OH, oxo, —SH, —CN, —NH₂, —CONH₂, C₁₋₆ alkyl, C₂₋₆ alkenyl, C₂₋₆ alkynyl, C₁₋₆ haloalkyl, —(C₁₋₆ alkyl)-O—(C₁₋₆ alkyl), —(C₁₋₆ alkyl)-OH, —(C₁₋₆ alkyl)-CN, —O—(C₁₋₆ alkyl), —O—(C₁₋₆ haloalkyl), —O—(C₃₋₈ cycloalkyl), —O-(4-8 membered heterocyclyl), —S—(C₁₋₆ alkyl), —NH(C₁₋₆ alkyl), —N(C₁₋₆ alkyl)₂, —CO(C₁₋₆ alkyl), —CO(C₂₋₆ alkenyl), —CO(C₂₋₆ alkynyl), —CONH(C₁₋₆ alkyl), —CON(C₁₋₆ alkyl)₂, C₃₋₈ cycloalkyl and 4-8 membered heterocyclyl;
L is absent, or L is C₁₋₆ alkyl or CO;
Rₐ, R_{b}, R_{c}, R_{d}, Rₑ and R_{f} are each independently selected from hydrogen, C₁₋₆ alkyl, C₂₋₆ alkenyl, C₂₋₆ alkynyl, —(C₁₋₆ alkyl)ₘ-(C₃₋₈ cycloalkyl), —(C₁₋₆ alkyl)ₘ-(4-8 membered heterocyclyl), —(C₁₋₆ alkyl)ₘ-phenyl and —(C₁₋₆ alkyl)ₘ-(5-12 membered heteroaryl), wherein the C₁₋₆ alkyl, C₃₋₈ cycloalkyl, 4-8 membered heterocyclyl, phenyl and 5-12 membered heteroaryl are each optionally substituted with one or more groups independently selected from halogen, —OH, oxo, —SH, —CN, —NH₂, —CONH₂, C₁₋₆ alkyl, C₂₋₆ alkenyl, C₂₋₆ alkynyl, C₁₋₆ haloalkyl, —(C₁₋₆ alkyl)-O—(C₁₋₆ alkyl), —(C₁₋₆ alkyl)-OH, —(C₁₋₆ alkyl)-CN, —O—(C₁₋₆ alkyl), —O—(C₁₋₆ haloalkyl), —O—(C₃₋₈ cycloalkyl), —O-(4-8 membered heterocyclyl), —S—(C₁₋₆ alkyl), —NH(C₁₋₆ alkyl), —N(C₁₋₆ alkyl)₂, —NH(C₃₋₈ cycloalkyl), —NH(4-8 membered heterocyclyl), —CO(C₁₋₆ alkyl), —CO(C₂₋₆ alkenyl), —CO(C₂₋₆ alkynyl), —CO(C₃₋₈ cycloalkyl), —CO(4-8 membered heterocyclyl), —CONH(C₁₋₆ alkyl), —CONH(C₃₋₈ cycloalkyl), —CONH(4-8 membered heterocyclyl) and —CON(C₁₋₆ alkyl)₂;
p is 0, 1, 2, 3, 4 or 5;
m is 0 or 1; and
n is 1 or 2.

Embodiment 1.1. The compound or the pharmaceutically acceptable salt, the solvate, the racemic mixture, the enantiomer, the diastereomer or the tautomer thereof, or the deuterate thereof according to embodiment 1, wherein Cy₂ is selected from C₃₋₈ cycloalkyl, 4-12 membered monocyclic or fused or bridged heterocyclyl, aryl and 5-14 membered heteroaryl; preferably, Cy₂ is selected from C₃₋₈ cycloalkyl, 4-9 membered heterocyclyl, aryl and 5-14 membered heteroaryl.

Embodiment 1.2. The compound or the pharmaceutically acceptable salt, the solvate, the racemic mixture, the enantiomer, the diastereomer or the tautomer thereof, or the deuterate thereof according to embodiment 1 or 1.1, wherein L is absent, or L is CH₂.

Embodiment 1.3. The compound or the pharmaceutically acceptable salt, the solvate, the racemic mixture, the enantiomer, the diastereomer or the tautomer thereof, or the deuterate thereof according to any one of embodiments 1-1.2, wherein R_{f} is —CH₃.

Embodiment 1.4. The compound or the pharmaceutically acceptable salt, the solvate, the racemic mixture, the enantiomer, the diastereomer or the tautomer thereof, or the deuterate thereof according to any one of embodiments 1-1.3, wherein provided that, R₄ is not —NH(6-membered nitrogen-containing heteroaryl) or —NH(phenyl substituted with F), further, R₄ is not —NH(6-membered heteroaryl) or —NH(phenyl substituted with F).

Embodiment 2. The compound or the pharmaceutically acceptable salt, the solvate, the racemic mixture, the enantiomer, the diastereomer or the tautomer thereof, or the deuterate thereof according to any one of embodiments 1-1.4, wherein
R₁ is halogen, CN or C₁₋₆ haloalkyl;
R₂ is selected from hydrogen, —O—(C₁₋₆ alkyl), C₃₋₈ cycloalkyl, 4-8 membered heterocyclyl, 5-12 membered heteroaryl, —CONRₐR_{b}, —CSNRₐR_{b}, —COR_{c} and —COORₑ, wherein the C₃₋₈ cycloalkyl, 4-8 membered heterocyclyl and 5-12 membered heteroaryl are each optionally substituted with one or more groups independently selected from halogen, —OH, oxo, —CN, —NH₂, —CONH₂, C₁₋₆ alkyl, C₁₋₆ haloalkyl, —(C₁₋₆ alkyl)-O—(C₁₋₆ alkyl), —(C₁₋₆ alkyl)-OH, —(C₁₋₆ alkyl)-CN, —O—(C₁₋₆ alkyl) and —O—(C₁₋₆ haloalkyl);
R₃ is hydrogen;
or R₂ and R₃ together with the carbon atom to which they are attached form 5-6 membered heteroaryl, wherein the 5-6 membered heteroaryl is optionally substituted with one or more groups independently selected from C₁₋₆ alkyl;
R₅ is selected from hydrogen, C₁₋₆ alkyl and —NRₐR_{b};
Cy₁ is 4-12 membered heterocyclyl;
Cy₂ is selected from C₃₋ₛ cycloalkyl, 4-9 membered heterocyclyl, aryl and 5-14 membered heteroaryl;
R₄ is independently selected from halogen, —CN, —OH, oxo, C₁₋₆ alkyl, —O—(C₁₋₆ alkyl), —(C₁₋₆ alkyl)ₘ-(C₃₋₈ cycloalkyl), —(C₁₋₆ alkyl)ₘ-(4-8 membered heterocyclyl), —(C₁₋₆ alkyl)ₘ-phenyl, —(C₁₋₆ alkyl)ₘ-(5-12 membered heteroaryl), —CONRₐR_{b}, —CORₑ, —COORₑ, —NRₐR_{b}, —NR_{d}COR_{c}, —NR_{d}S(O)ₙR_{f}, —S(O)ₙR_{f} and —S(O)ₙNRₐR_{b}, wherein the C₁₋₆ alkyl, C₃₋₈ cycloalkyl, 4-8 membered heterocyclyl, phenyl and 5-12 membered heteroaryl are each optionally substituted with one or more groups independently selected from halogen, —OH, oxo, —CN, —NH₂, —CONH₂, C₁₋₆ alkyl, C₂₋₆ alkenyl, C₂₋₆ alkynyl, C₁₋₆ haloalkyl, —(C₁₋₆ alkyl)-O—(C₁₋₆ alkyl), —(C₁₋₆ alkyl)-OH, —(C₁₋₆ alkyl)-CN, —O—(C₁₋₆ alkyl), —O—(C₁₋₆ haloalkyl), —NH(C₁₋₆ alkyl), —N(C₁₋₆ alkyl)₂, —CONH(C₁₋₆ alkyl), —CON(C₁₋₆ alkyl)₂, C₃₋₈ cycloalkyl and 4-8 membered heterocyclyl;
L is absent, or L is CH₂;
Rₐ, R_{b}, R_{c}, R_{d} and Rₑ are each independently selected from hydrogen, C₁₋₆ alkyl, C₂₋₆ alkenyl, —(C₁₋₆ alkyl)ₘ-(C₃₋₈ cycloalkyl), —(C₁₋₆ alkyl)ₘ-(4-8 membered heterocyclyl), —(C₁₋₆ alkyl)ₘ-phenyl and —(C₁₋₆ alkyl)ₘ-(5-12 membered heteroaryl), wherein the C₁₋₆ alkyl, C₃₋₈ cycloalkyl, 4-8 membered heterocyclyl, phenyl and 5-12 membered heteroaryl are each optionally substituted with one or more groups independently selected from halogen, —OH, —CN, —CONH₂, C₁₋₆ alkyl, C₁₋₆ haloalkyl, —(C₁₋₆ alkyl)-OH, —O—(C₁₋₆ alkyl), —NH(C₃₋₈ cycloalkyl), —CO(C₂₋₆ alkenyl) and —CON(C₁₋₆ alkyl)₂;
R_{f} is —CH₃;
p is 0, 1, 2 or 3;
m is 0 or 1; and
n is 1 or 2;
provided that, R₄ is not —NH(6-membered nitrogen-containing heteroaryl) or —NH(phenyl substituted with F), further, R₄ is not —NH(6-membered heteroaryl) or —NH(phenyl substituted with F).

Embodiment 3. The compound or the pharmaceutically acceptable salt, the solvate, the racemic mixture, the enantiomer, the diastereomer or the tautomer thereof, or the deuterate thereof according to any one of embodiments 1-2, wherein the compound is a compound of formula (I-1):

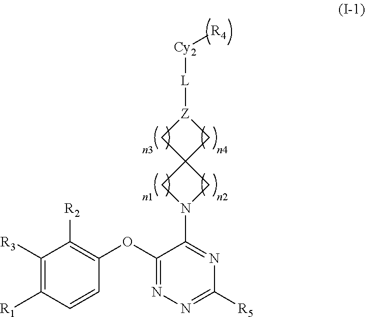

wherein
Z is N or CH; preferably, Z is N; and
n1, n2, n3 and n4 are each independently selected from 1 and 2.

Embodiment 4. The compound or the pharmaceutically acceptable salt, the solvate, the racemic mixture, the enantiomer, the diastereomer or the tautomer thereof, or the deuterate thereof according to any one of embodiments 1-2, wherein the compound is a compound of formula (I-2):

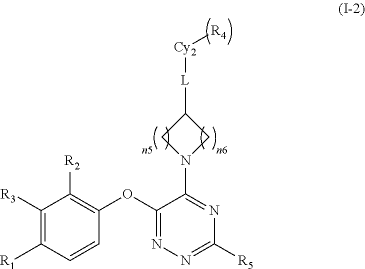

wherein n5 and n6 are each independently selected from 1 and 2.

Embodiment 5. The compound or the pharmaceutically acceptable salt, the solvate, the racemic mixture, the enantiomer, the diastereomer or the tautomer thereof, or the deuterate thereof according to any one of embodiments 1-4, wherein R₁ is halogen; preferably, R₁ is F or Cl; and more preferably, R₁ is F.

Embodiment 6. The compound or the pharmaceutically acceptable salt, the solvate, the racemic mixture, the enantiomer, the diastereomer or the tautomer thereof, or the deuterate thereof according to any one of embodiments 1-5, wherein R₂ is selected from hydrogen, —O—(C₁₋₆ alkyl), C₃₋₈ cycloalkyl, 4-8 membered heterocyclyl, 5-12 membered heteroaryl, —CONRₐR_{b}, —CSNRₐR_{b}, —COR_{c} and —COORₑ, wherein Rₐ, R_{b}, R_{c} and Rₑ are each independently selected from hydrogen, C₁₋₆ alkyl, C₁₋₆ haloalkyl, —(C₁₋₆ alkyl)-O—(C₁₋₆ alkyl), —(C₁₋₆ alkyl)-OH, —(C₁₋₆ alkyl)-CN, C₃₋₈ cycloalkyl and 4-8 membered heterocyclyl, wherein the C₃₋₈ cycloalkyl, 4-8 membered heterocyclyl and 5-12 membered heteroaryl are each optionally substituted with one or more groups independently selected from halogen, —OH, oxo, —CN, —NH₂, —CONH₂, C₁₋₆ alkyl, C₁₋₆ haloalkyl, —(C₁₋₆ alkyl)-O—(C₁₋₆ alkyl), —(C₁₋₆ alkyl)-OH, —(C₁₋₆ alkyl)-CN, —O—(C₁₋₆ alkyl) and —O—(C₁₋₆ haloalkyl);
preferably, R₂ is selected from hydrogen, —O—(C₁₋₆ alkyl), C₃₋₆ cycloalkyl, 4-6 membered heterocyclyl, 5-6 membered heteroaryl, —CONRₐR_{b}, —CSNRₐR_{b}, —COR_{c} and —COORₑ, wherein Rₐ, R_{b}, R_{c} and Rₑ are each independently selected from C₁₋₆ alkyl, C₃₋₆ cycloalkyl and 4-6 membered heterocyclyl, wherein the C₃₋₆ cycloalkyl, 4-6 membered heterocyclyl and 5-6 membered heteroaryl are each optionally substituted with one or more groups independently selected from halogen, —OH and C₁₋₆ alkyl;
more preferably, R₂ is selected from —COO(C₁₋₆ alkyl) and —CON(C₁₋₆ alkyl)₂; and
most preferably, R₂ is —CON(C₁₋₆ alkyl)₂.

Embodiment 7. The compound or the pharmaceutically acceptable salt, the solvate, the racemic mixture, the enantiomer, the diastereomer or the tautomer thereof, or the deuterate thereof according to embodiment 1, wherein R₃ is hydrogen.

Embodiment 8. The compound or the pharmaceutically acceptable salt, the solvate, the racemic mixture, the enantiomer, the diastereomer or the tautomer thereof, or the deuterate thereof according to any one of embodiments 1-5, wherein R₂ and R₃ together with the carbon atom to which they are attached form 5-6 membered heteroaryl, wherein the 5-6 membered heteroaryl is optionally substituted with one or more groups independently selected from C₁₋₆ alkyl; preferably, R₂ and R₃ together with the carbon atom to which they are attached form pyrazolyl, wherein the pyrazolyl is optionally substituted with one or more groups independently selected from C₁₋₆ alkyl.

Embodiment 9. The compound or the pharmaceutically acceptable salt, the solvate, the racemic mixture, the enantiomer, the diastereomer or the tautomer thereof, or the deuterate thereof according to any one of embodiments 1-8, wherein R₅ is selected from hydrogen, C₁₋₆ alkyl, —NH₂, —NH(C₁₋₆ alkyl) and —N(C₁₋₆ alkyl)₂; preferably, R₅ is selected from hydrogen, C₁₋₆ alkyl and —NH(C₁₋₆ alkyl); and more preferably, R₅ is hydrogen.

Embodiment 10. The compound or the pharmaceutically acceptable salt, the solvate, the racemic mixture, the enantiomer, the diastereomer or the tautomer thereof, or the deuterate thereof according to any one of embodiments 1-9, wherein L is CH₂.

Embodiment 11. The compound or the pharmaceutically acceptable salt, the solvate, the racemic mixture, the enantiomer, the diastereomer or the tautomer thereof, or the deuterate thereof according to any one of embodiments 1-10, wherein Cy₂ is selected from C₃₋₈ cycloalkyl, 4-8 membered heterocyclyl, phenyl and 5-12 membered heteroaryl; preferably, Cy₂ is selected from C₃₋₆ cycloalkyl, 4-6 membered heterocyclyl, phenyl, 5-6 membered heteroaryl and 8-10 membered heteroaryl.

Embodiment 12. The compound or the pharmaceutically acceptable salt, the solvate, the racemic mixture, the enantiomer, the diastereomer or the tautomer thereof, or the deuterate thereof according to embodiment 11, wherein Cy₂ is selected from cyclopropyl, cyclobutyl, cyclohexyl, cyclohexenyl, pyrrolidyl, piperidyl, piperazinyl, phenyl, pyridyl, pyridinonyl, pyrimidyl, indolyl, indazolyl, benzofuranyl, benzoxazolyl, imidazopyridyl, quinolinyl, quinolinonyl, quinazolinyl and dihydro-[1,4]dioxinopyridyl.

Embodiment 13. The compound or the pharmaceutically acceptable salt, the solvate, the racemic mixture, the enantiomer, the diastereomer or the tautomer thereof, or the deuterate thereof according to embodiment 12, wherein Cy₂ is selected from

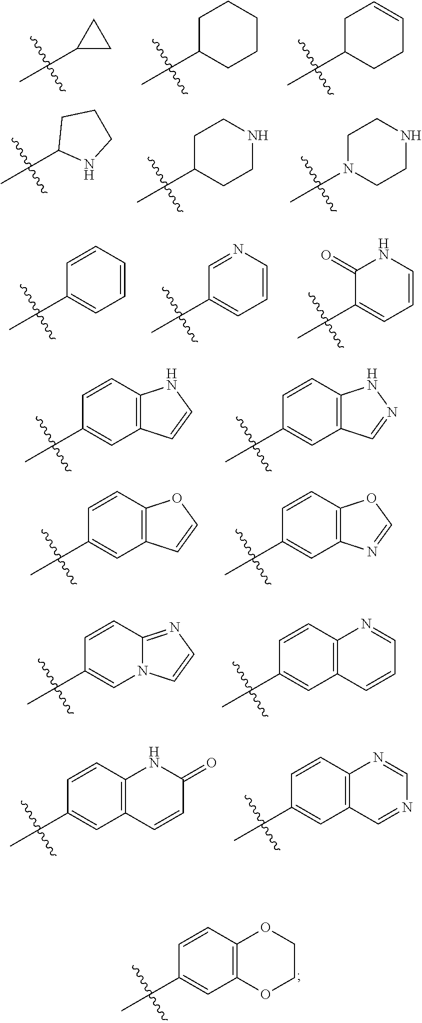

preferably, Cy₂ is selected from

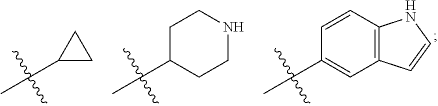

and more preferably, Cy₂ is

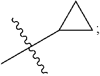 [SMILES: *C1CC1]

### or Cy₂ is

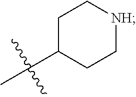 [SMILES: *C1CCNCC1]

### or Cy₂ is

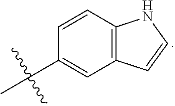 [SMILES: *c1ccc2[nH]ccc2c1]

Embodiment 14. The compound or the pharmaceutically acceptable salt, the solvate, the racemic mixture, the enantiomer, the diastereomer or the tautomer thereof, or the deuterate thereof according to any one of embodiments 1-13, wherein R₄ is independently selected from halogen, —CN, —OH, oxo, C₁₋₆ alkyl, —O—(C₁₋₆ alkyl), —(C₁₋₆ alkyl)ₘ, —(C₃₋₈ cycloalkyl), —(C₁₋₆ alkyl)ₘ-(4-8 membered heterocyclyl), —(C₁₋₆ alkyl)ₘ-phenyl, —(C₁₋₆ alkyl)ₘ-(5-12 membered heteroaryl), —CONRₐR_{b}, —COR_{c}, —COORₑ, —NRₐR_{b}, —NR_{d}COR_{c}, —NR_{d}S(O)ₙR_{f} and —S(O)ₙR_{f}, wherein the C₁₋₆ alkyl, C₃₋₈ cycloalkyl, 4-8 membered heterocyclyl, phenyl and 5-12 membered heteroaryl are each optionally substituted with one or more groups independently selected from halogen, —OH, oxo, —CN, —NH₂, —CONH₂, C₁₋₆ alkyl, C₂₋₆ alkenyl, C₂₋₆ alkynyl, C₁₋₆ haloalkyl, —(C₁₋₆ alkyl)-O—(C₁₋₆ alkyl), —(C₁₋₆ alkyl)-OH, —(C₁₋₆ alkyl)-CN, —O—(C₁₋₆ alkyl), —O—(C₁₋₆ haloalkyl), —NH(C₁₋₆ alkyl), —N(C₁₋₆ alkyl)₂, —CONH(C₁₋₆ alkyl), —CON(C₁₋₆ alkyl)₂, C₃₋₈ cycloalkyl and 4-8 membered heterocyclyl;
preferably, R₄ is independently selected from halogen, —CN, —OH, oxo, C₁₋₆ alkyl, —O—(C₁₋₆ alkyl), —(C₁₋₆ alkyl)ₘ-(4-8 membered heterocyclyl), —(C₁₋₆ alkyl)ₘ-phenyl, —(C₁₋₆ alkyl)ₘ-(5-12 membered heteroaryl), —CONRₐR_{b}, —COR_{c}, —COORₑ, —NRₐR_{b}, —NR_{d}COR_{c}, —NR_{d}S(O)ₙR_{f} and —S(O)ₙR_{f}, wherein the C₁₋₆ alkyl, 4-8 membered heterocyclyl, phenyl and 5-12 membered heteroaryl are each optionally substituted with one or more groups independently selected from halogen, —OH, oxo, —CN, —NH₂, —CONH₂, C₁₋₆ alkyl, C₂₋₆ alkenyl, C₂₋₆ alkynyl, C₁₋₆ haloalkyl, —(C₁₋₆ alkyl)-OH, —O—(C₁₋₆ alkyl) and C₃₋₈ cycloalkyl;
provided that, R₄ is not —NH(6-membered nitrogen-containing heteroaryl) or —NH(phenyl substituted with F), further, R₄ is not —NH(6-membered heteroaryl) or —NH(phenyl substituted with F).

Embodiment 15. The compound or the pharmaceutically acceptable salt, the solvate, the racemic mixture, the enantiomer, the diastereomer or the tautomer thereof, or the deuterate thereof according to embodiment 14, wherein R₄ is independently selected from:
1) halogen;
2) —CN;
3) —OH;
4) oxo;
5) C₁₋₆ alkyl, which is optionally substituted with one or more groups independently selected from —OH, —CN and —CONH₂;
6) —O—(C₁₋₆ alkyl);
7) —(C₁₋₆ alkyl)ₘ-(4-6 membered heterocyclyl), wherein preferably, the 4-6 membered heterocyclyl is selected from oxetanyl and dihydropyranyl;
8) —(C₁₋₆ alkyl)ₘ-phenyl, wherein the phenyl is optionally substituted with one or more groups independently selected from —CN;
9) —(C₁₋₆ alkyl)ₘ-(5-12 membered heteroaryl), wherein preferably, the 5-12 membered heteroaryl is selected from 5-6 membered heteroaryl and 8-10 membered heteroaryl; more preferably, the 5-12 membered heteroaryl is selected from pyrazolyl, oxazolyl, pyridyl, pyridinonyl, pyrimidyl, pyridazinyl, pyridazinonyl, pyrazinyl, 1,2,4-triazinyl, indolyl, imidazopyridazinyl, [1,2,4]triazolopyridyl, pyrazolopyrimidyl, dihydro-pyrimidopyridazinyl and dihydro-[1,4]dioxinopyridazinyl; and most preferably, the 5-12 membered heteroaryl is selected from pyridyl and pyridazinyl;
wherein the 5-12 membered heteroaryl is optionally substituted with one or more groups independently selected from halogen, —OH, oxo, —CN, —NH₂, —CONH₂, C₁₋₆ alkyl, C₂₋₆ alkenyl, C₂₋₆ alkynyl, C₁₋₆ haloalkyl, —(C₁₋₆ alkyl)-OH, —O—(C₁₋₆ alkyl) and C₃₋₈ cycloalkyl; preferably, the 5-12 membered heteroaryl is optionally substituted with one or more groups independently selected from halogen, —OH, —CN, —CONH₂, C₁₋₆ alkyl, C₂₋₆ alkenyl, C₂₋₆ alkynyl, C₁₋₆ haloalkyl, —(C₁₋₆ alkyl)-OH, —O—(C₁₋₆ alkyl) and C₃₋₈ cycloalkyl;
10) —CONRₐR_{b}, wherein Rₐ and R_{b} are each independently selected from hydrogen, C₁₋₆ alkyl, —(C₁₋₆ alkyl)ₘ-(C₃₋₆ cycloalkyl), —(C₁₋₆ alkyl)ₘ-(4-6 membered heterocyclyl), —(C₁₋₆ alkyl)ₘ-phenyl and —(C₁₋₆ alkyl)ₘ-(5-6 membered heteroaryl), wherein the C₁₋₆ alkyl, C₃₋₆ cycloalkyl, 4-6 membered heterocyclyl, phenyl and 5-6 membered heteroaryl are each optionally substituted with one or more groups independently selected from halogen and —O—(C₁₋₆ alkyl);
11) —COR_{c}, wherein R_{c} is selected from C₁₋₆ alkyl, C₂₋₆ alkenyl, —(C₁₋₆ alkyl)ₘ-(C₃₋₈ cycloalkyl), —(C₁₋₆ alkyl)ₘ-(4-6 membered heterocyclyl), —(C₁₋₆ alkyl)ₘ-phenyl and —(C₁₋₆ alkyl)ₘ-(5-10 membered heteroaryl), wherein the C₁₋₆ alkyl, C₃₋₈ cycloalkyl, 4-6 membered heterocyclyl, phenyl and 5-10 membered heteroaryl are each optionally substituted with one or more groups independently selected from halogen, —OH, —CN, C₁₋₆ alkyl, C₁₋₆ haloalkyl, —O—(C₁₋₆ alkyl), —NH(C₃₋₆ cycloalkyl), —CO(C₂₋₆ alkenyl) and —CON(C₁₋₆ alkyl)₂;
12) —COOR_{c}, wherein R_{c} is selected from hydrogen and C₁₋₆ alkyl;
13) —NRₐR_{b}, wherein Rₐ and R_{b} are each independently selected from hydrogen, C₁₋₆ alkyl and —(C₁₋₆ alkyl)ₘ-(5-10 membered heteroaryl), wherein the C₁₋₆ alkyl and 5-10 membered heteroaryl are each optionally substituted with one or more groups independently selected from halogen, —OH, —CN, —CONH₂, C₁₋₆ alkyl, —(C₁₋₆ alkyl)-OH and —O—(C₁₋₆ alkyl), provided that, R₄ is not —NH(6-membered nitrogen-containing heteroaryl), further, R₄ is not —NH(6-membered heteroaryl);
14) —NR_{d}COR_{c}, wherein R_{d} is selected from hydrogen and C₁₋₆ alkyl; R_{c} is C₁₋₆ alkyl, which is optionally substituted with one or more groups independently selected from —CN and —O—(C₁₋₆ alkyl);
15) —NR_{d}S(O)ₙR_{f}, wherein R_{d} is hydrogen; R_{f} is —CH₃;
16) —S(O)ₙR_{f}, wherein R_{f} is —CH₃.

Embodiment 16. The compound or the pharmaceutically acceptable salt, the solvate, the racemic mixture, the enantiomer, the diastereomer or the tautomer thereof, or the deuterate thereof according to embodiment 1, wherein the compound is a compound of formula (I-3):

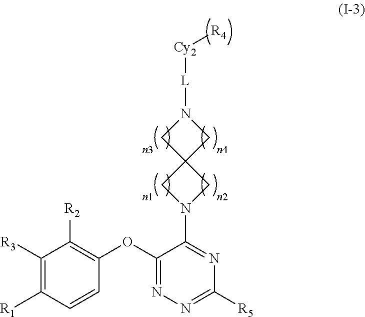

wherein
n1, n2, n3 and n4 are each independently selected from 1 and 2; preferably, both n1 and n2 are 1, and both n3 and n4 are 2;
R₁ is halogen; preferably, R₁ is F or Cl; and more preferably, R₁ is F;
R₂ is selected from hydrogen, —O—(C₁₋₆ alkyl), C₃₋₈ cycloalkyl, 4-8 membered heterocyclyl, 5-12 membered heteroaryl, —CONRₐR_{b}, —CSNRₐR_{b}, —CORₑ and —COOR_{c}, wherein Rₐ, R_{b}, R_{c} and R_{c} are each independently selected from hydrogen, C₁₋₆ alkyl, C₁₋₆ haloalkyl, —(C₁₋₆ alkyl)-O—(C₁₋₆ alkyl), —(C₁₋₆ alkyl)-OH, —(C₁₋₆ alkyl)-CN, C₃₋₈ cycloalkyl and 4-8 membered heterocyclyl, wherein the C₃₋₈ cycloalkyl, 4-8 membered heterocyclyl and 5-12 membered heteroaryl are each optionally substituted with one or more groups independently selected from halogen, —OH, oxo, —CN, —NH₂, —CONH₂, C₁₋₆ alkyl, C₁₋₆ haloalkyl, —(C₁₋₆ alkyl)-O—(C₆ alkyl), —(C₁₋₆ alkyl)-OH, —(C₁₋₆ alkyl)-CN, —O—(C₁₋₆ alkyl) and —O—(C₁₋₆ haloalkyl); preferably, R₂ is selected from hydrogen, —O—(C₁₋₆ alkyl), C₃₋₆ cycloalkyl, 4-6 membered heterocyclyl, 5-6 membered heteroaryl, —CONRₐR_{b}, —CSNRₐR_{b}, —COR_{c} and —COORₑ, wherein Rₐ, R_{b}, R_{c} and Rₑ are each independently selected from C₁₋₆ alkyl, C₃₋₆ cycloalkyl and 4-6 membered heterocyclyl, wherein the C₃₋₆ cycloalkyl, 4-6 membered heterocyclyl and 5-6 membered heteroaryl are each optionally substituted with one or more groups independently selected from halogen, —OH and C₁₋₆ alkyl; more preferably, R₂ is selected from —COO(C₁₋₆ alkyl) and —CON(C₁₋₆ alkyl)₂; and most preferably, R₂ is —CON(C₁₋₆ alkyl)₂;
R₃ is hydrogen;
or R₂ and R₃ together with the carbon atom to which they are attached form 5-6 membered heteroaryl, wherein the 5-6 membered heteroaryl is optionally substituted with one or more groups independently selected from C₁₋₆ alkyl; preferably, R₂ and R₃ together with the carbon atom to which they are attached form pyrazolyl, wherein the pyrazolyl is optionally substituted with one or more groups independently selected from C₁₋₆ alkyl;
R₅ is selected from hydrogen, C₁₋₆ alkyl, —NH₂, —NH(C₁₋₆ alkyl) and —N(C₁₋₆ alkyl)₂; preferably, R₅ is selected from hydrogen, C₁₋₆ alkyl and —NH(C₁₋₆ alkyl); and more preferably, R₅ is hydrogen;
Cy₂ is selected from C₃₋₆ cycloalkyl, 4-6 membered heterocyclyl, phenyl, 5-6 membered heteroaryl and 8-10 membered heteroaryl; preferably, Cy₂ is selected from cyclopropyl, cyclobutyl, cyclohexyl, cyclohexenyl, pyrrolidyl, piperidyl, piperazinyl, phenyl, pyridyl, pyridinonyl, pyrimidyl, indolyl, indazolyl, benzofuranyl, benzoxazolyl, imidazopyridyl, quinolinyl, quinolinonyl, quinazolinyl and dihydro-[1,4]dioxinopyridyl; more preferably, Cy₂ is selected from

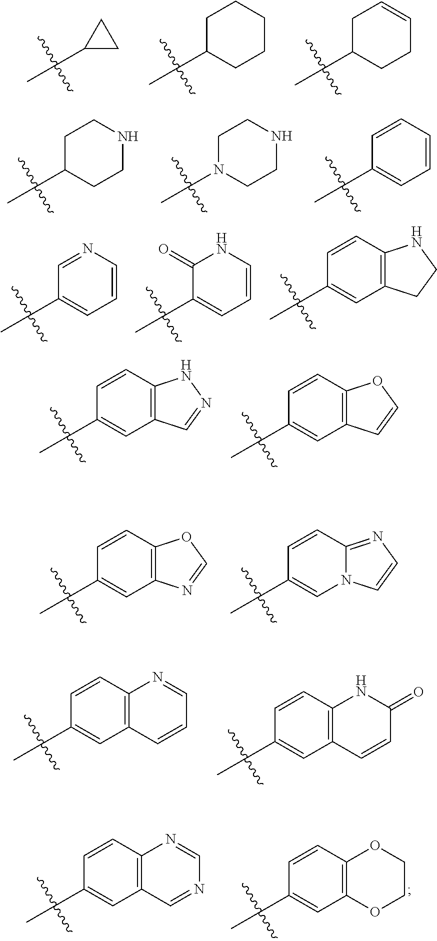

and most preferably, Cy₂ is

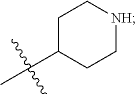 [SMILES: *C1CCNCC1]

R₄ is independently selected from halogen, —CN, —OH, oxo, C₁₋₆ alkyl, —O—(C₁₋₆ alkyl), —(C₁₋₆ alkyl)ₘ-(4-8 membered heterocyclyl), —(C₁₋₆ alkyl)ₘ-phenyl, —(C₁₋₆ alkyl)ₘ-(5-12 membered heteroaryl), —CONRₐR_{b}, —COR_{c}, —COORₑ, —NRₐR_{b}, —NR_{d}COR_{c}, —NR_{d}S(O)ₙR_{f} and —S(O)ₙR_{f}, wherein the C₁₋₆ alkyl, 4-8 membered heterocyclyl, phenyl and 5-12 membered heteroaryl are each optionally substituted with one or more groups independently selected from halogen, —OH, oxo, —CN, —NH₂, —CONH₂, C₁₋₆ alkyl, C₂₋₆ alkenyl, C₂₋₆ alkynyl, C₁₋₆ haloalkyl, —(C₁₋₆ alkyl)-OH, —O—(C₁₋₆ alkyl) and C₃₋₈ cycloalkyl; preferably, R₄ is independently selected from:
1) halogen;
2) —CN;
3) —OH;
4) oxo;
5) C₁₋₆ alkyl, which is optionally substituted with one or more groups independently selected from —OH, —CN and —CONH₂;
6) —O—(C₁₋₆ alkyl);
7) —(C₁₋₆ alkyl)ₘ-(4-6 membered heterocyclyl), wherein preferably, the 4-6 membered heterocyclyl is selected from oxetanyl and dihydropyranyl;
8) —(C₁₋₆ alkyl)ₘ-phenyl, wherein the phenyl is optionally substituted with one or more groups independently selected from —CN;
9) —(C₁₋₆ alkyl)ₘ-(5-12 membered heteroaryl), wherein preferably, the 5-12 membered heteroaryl is selected from 5-6 membered heteroaryl and 8-10 membered heteroaryl; more preferably, the 5-12 membered heteroaryl is selected from pyrazolyl, oxazolyl, pyridyl, pyridinonyl, pyrimidyl, pyridazinyl, pyridazinonyl, pyrazinyl, 1,2,4-triazinyl, indolyl, imidazopyridazinyl, [1,2,4]triazolopyridyl, pyrazolopyrimidyl, dihydro-pyrimidopyridazinyl and dihydro-[1,4]dioxinopyridazinyl; and most preferably, the 5-12 membered heteroaryl is selected from pyridyl and pyridazinyl;
wherein the 5-12 membered heteroaryl is optionally substituted with one or more groups independently selected from halogen, —OH, oxo, —CN, —NH₂, —CONH₂, C₁₋₆ alkyl, C₂₋₆ alkenyl, C₂₋₆ alkynyl, C₁₋₆ haloalkyl, —(C₁₋₆ alkyl)-OH, —O—(C₁₋₆ alkyl) and C₃₋₈ cycloalkyl; preferably, the 5-12 membered heteroaryl is optionally substituted with one or more groups independently selected from halogen, —OH, —CN, —CONH₂, C₁₋₆ alkyl, C₂₋₆ alkenyl, C₂₋₆ alkynyl, C₆ haloalkyl, —(C₁₋₆ alkyl)-OH, —O—(C₁₋₆ alkyl) and C₃₋₈ cycloalkyl;
10) —CONRₐR_{b}, wherein Rₐ and R_{b} are each independently selected from hydrogen, C₁₋₆ alkyl, —(C₁₋₆ alkyl)ₘ-(C₃₋₆ cycloalkyl), —(C₁₋₆ alkyl)ₘ-(4-6 membered heterocyclyl), —(C₁₋₆ alkyl)ₘ-phenyl and —(C₁₋₆ alkyl)ₘ-(5-6 membered heteroaryl), wherein the C₁₋₆ alkyl, C₃₋₆ cycloalkyl, 4-6 membered heterocyclyl, phenyl and 5-6 membered heteroaryl are each optionally substituted with one or more groups independently selected from halogen and —O—(C₁₋₆ alkyl);
11) —COR_{c}, wherein R_{c} is selected from C₁₋₆ alkyl, C₂₋₆ alkenyl, —(C₁₋₆ alkyl)ₘ-(C₃₋₈ cycloalkyl), —(C₁₋₆ alkyl)ₘ-(4-6 membered heterocyclyl), —(C₁₋₆ alkyl)ₘ-phenyl and —(C₁₋₆ alkyl)ₘ-(5-10 membered heteroaryl), wherein the C₁₋₆ alkyl, C₃₋₈ cycloalkyl, 4-6 membered heterocyclyl, phenyl and 5-10 membered heteroaryl are each optionally substituted with one or more groups independently selected from halogen, —OH, —CN, C₁₋₆ alkyl, C₁₋₆ haloalkyl, —O—(C₁₋₆ alkyl), —NH(C₃₋₆ cycloalkyl), —CO(C₂₋₆ alkenyl) and —CON(C₁₋₆ alkyl)₂;
12) —COORₑ, wherein Rₑ is selected from hydrogen and C₁₋₆ alkyl;
13) —NRₐR_{b}, wherein Rₐ and R_{b} are each independently selected from hydrogen and C₁₋₆ alkyl, wherein the C₁₋₆ alkyl is optionally substituted with one or more groups independently selected from —CN and —O—(C₁₋₆ alkyl);
14) —NR_{d}COR_{c}, wherein R_{d} is selected from hydrogen and C₁₋₆ alkyl; R_{c} is C₁₋₆ alkyl, which is optionally substituted with one or more groups independently selected from —CN and —O—(C₁₋₆ alkyl);
15) —NR_{d}S(O)ₙR_{f}, wherein R_{d} is hydrogen; R_{f} is —CH₃;
16) —S(O)ₙR_{f}, wherein R_{f} is —CH₃;
L is CH₂;
p is 0, 1, 2 or 3;
m is 0 or 1; and
n is 1 or 2; preferably, n is 2.

Embodiment 17. The compound or the pharmaceutically acceptable salt, the solvate, the racemic mixture, the enantiomer, the diastereomer or the tautomer thereof, or the deuterate thereof according to embodiment 1, wherein the compound is a compound of formula (I-4):

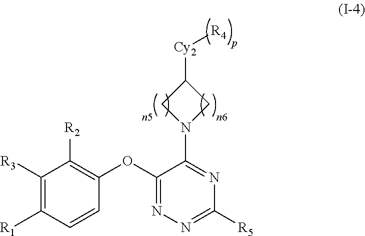

wherein
n5 and n6 are each independently selected from 1 and 2; preferably, both n5 and n6 are 1;
R₁ is halogen; preferably, R₁ is F;
R₂ is —CON(C₁₋₆ alkyl)₂;
R₃ is hydrogen;
R₅ is hydrogen;
Cy₂ is selected from C₃₋₉ cycloalkyl and 4-8 membered heterocyclyl; preferably, Cy₂ is selected from cyclopropyl and pyrrolidyl; more preferably, Cy₂ is selected from

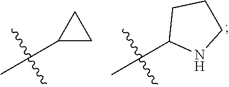

and most preferably, Cy₂ is

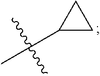 [SMILES: *C1CC1]

R₄ is independently selected from —(C₁₋₆ alkyl)ₘ-(5-12 membered heteroaryl) and —NRₐR_{b}, wherein the 5-12 membered heteroaryl is optionally substituted with one or more groups independently selected from —CN and C₁₋₆ alkyl; preferably, R₄ is independently selected from:
1) —(C₁₋₆ alkyl)-indolyl, wherein the indolyl is optionally substituted with one or more groups independently selected from —CN and C₁₋₆ alkyl;
2) —NRₐR_{b}, wherein Rₐ and R_{b} are each independently selected from hydrogen, C₁₋₆ alkyl and —(C₁₋₆ alkyl)ₘ-(5-10 membered heteroaryl), wherein the C₁₋₆ alkyl and 5-10 membered heteroaryl are each optionally substituted with one or more groups independently selected from —OH, —CN, C₁₋₆ alkyl and —(C₁₋₆ alkyl)-OH, provided that, R₄ is not —NH(6-membered nitrogen-containing heteroaryl), further, R₄ is not —NH(6-membered heteroaryl);
p is 0, 1 or 2; preferably, p is 1; and
m is 0 or 1.

Embodiment 18. A compound or the pharmaceutically acceptable salt, the solvate, the racemic mixture, the enantiomer, the diastereomer or the tautomer thereof, or the deuterate thereof, which is selected from:

| No. | Structural formula |
|---|---|
| 1 | 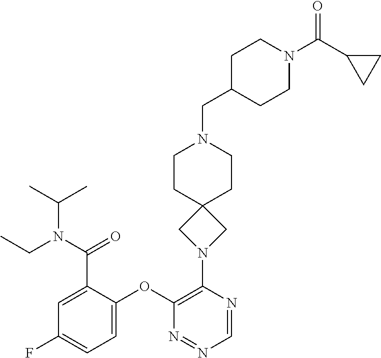 [SMILES: CCN(C(=O)c1cc(F)ccc1Oc1nncnc1N1CC2(CCN(CC3CCN(C(=O)C4CC4)CC3)CC2)C1)C(C)C] |
| 2 | 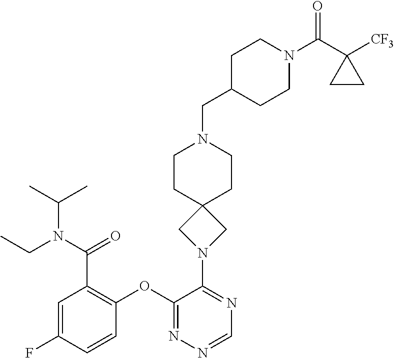 [SMILES: CCN(C(=O)c1cc(F)ccc1Oc1nncnc1N1CC2(CCN(CC3CCN(C(=O)C4(C(F)(F)F)CC4)CC3)CC2)C1)C(C)C] |
| 3 | 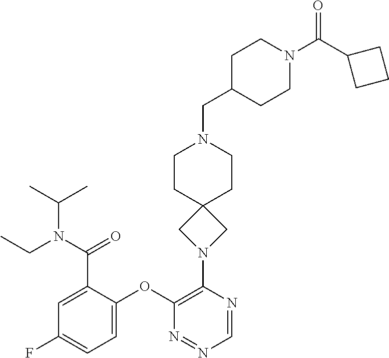 [SMILES: CCN(C(=O)c1cc(F)ccc1Oc1nncnc1N1CC2(CCN(CC3CCN(C(=O)C4CCC4)CC3)CC2)C1)C(C)C] |
| 4 | 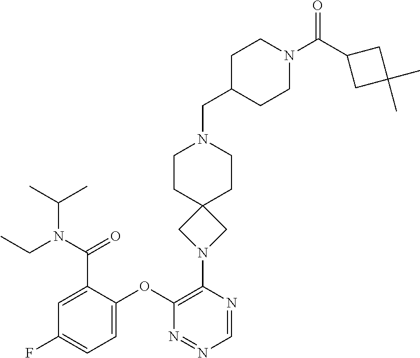 [SMILES: CCN(C(=O)c1cc(F)ccc1Oc1nncnc1N1CC2(CCN(CC3CCN(C(=O)C4CC(C)(C)C4)CC3)CC2)C1)C(C)C] |
| 5 | 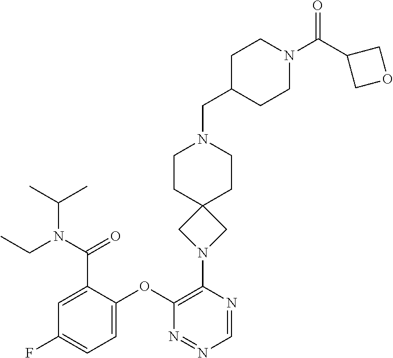 [SMILES: CCN(C(=O)c1cc(F)ccc1Oc1nncnc1N1CC2(CCN(CC3CCN(C(=O)C4COC4)CC3)CC2)C1)C(C)C] |
| 6 | 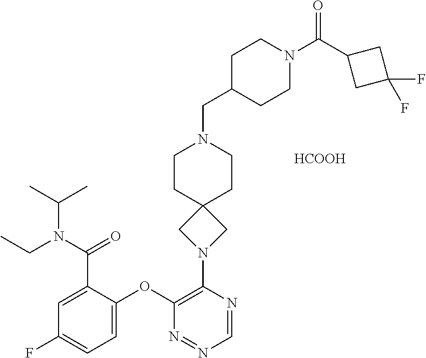 [SMILES: CCN(C(=O)c1cc(F)ccc1Oc1nncnc1N1CC2(CCN(CC3CCN(C(=O)C4CC(F)(F)C4)CC3)CC2)C1)C(C)C ; "HCOOH"] |
| 7 | 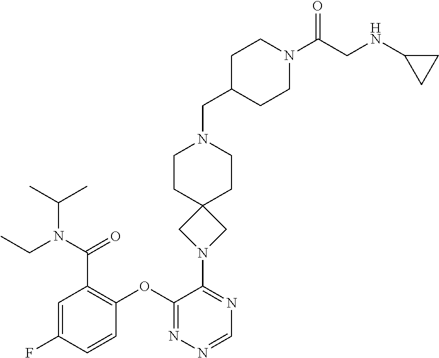 [SMILES: CCN(C(=O)c1cc(F)ccc1Oc1nncnc1N1CC2(CCN(CC3CCN(C(=O)CNC4CC4)CC3)CC2)C1)C(C)C] |
| 8 | 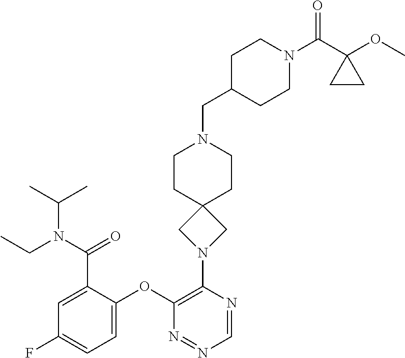 [SMILES: CCN(C(=O)c1cc(F)ccc1Oc1nncnc1N1CC2(CCN(CC3CCN(C(=O)C4(OC)CC4)CC3)CC2)C1)C(C)C] |
| 9 | 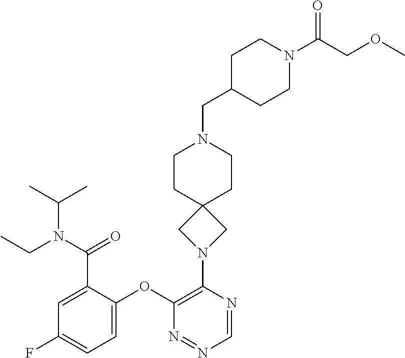 [SMILES: CCN(C(=O)c1cc(F)ccc1Oc1nncnc1N1CC2(CCN(CC3CCN(C(=O)COC)CC3)CC2)C1)C(C)C] |
| 10 | 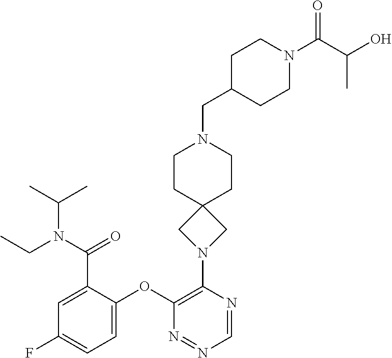 [SMILES: CCN(C(=O)c1cc(F)ccc1Oc1nncnc1N1CC2(CCN(CC3CCN(C(=O)C(C)O)CC3)CC2)C1)C(C)C] |
| 11 | 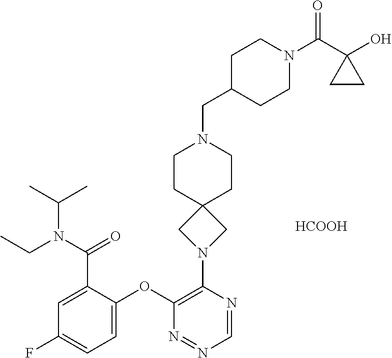 [SMILES: CCN(C(=O)c1cc(F)ccc1Oc1nncnc1N1CC2(CCN(CC3CCN(C(=O)C4(O)CC4)CC3)CC2)C1)C(C)C ; "HCOOH"] |
| 12 | 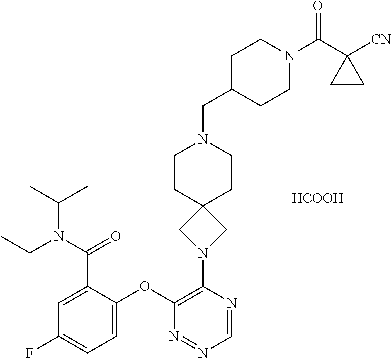 [SMILES: CCN(C(=O)c1cc(F)ccc1Oc1nncnc1N1CC2(CCN(CC3CCN(C(=O)C4(C#N)CC4)CC3)CC2)C1)C(C)C ; "HCOOH"] |
| 13 | 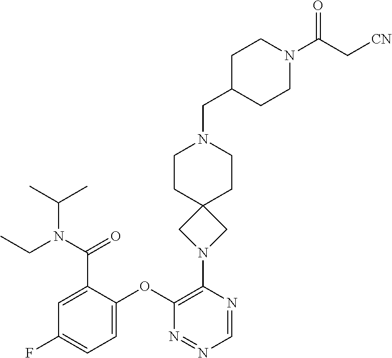 [SMILES: CCN(C(=O)c1cc(F)ccc1Oc1nncnc1N1CC2(CCN(CC3CCN(C(=O)CC#N)CC3)CC2)C1)C(C)C] |
| 14 | 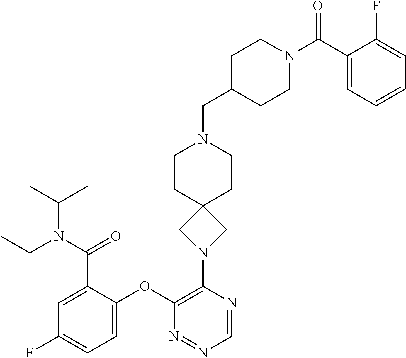 [SMILES: CCN(C(=O)c1cc(F)ccc1Oc1nncnc1N1CC2(CCN(CC3CCN(C(=O)c4ccccc4F)CC3)CC2)C1)C(C)C] |
| 15 | 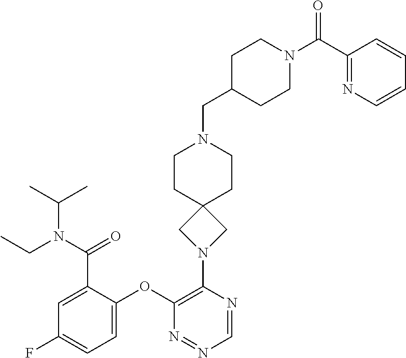 [SMILES: CCN(C(=O)c1cc(F)ccc1Oc1nncnc1N1CC2(CCN(CC3CCN(C(=O)c4ccccn4)CC3)CC2)C1)C(C)C] |
| 16 | 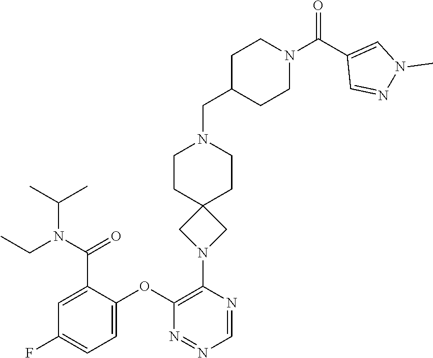 [SMILES: CCN(C(=O)c1cc(F)ccc1Oc1nncnc1N1CC2(CCN(CC3CCN(C(=O)c4cnn(C)c4)CC3)CC2)C1)C(C)C] |
| 17 | 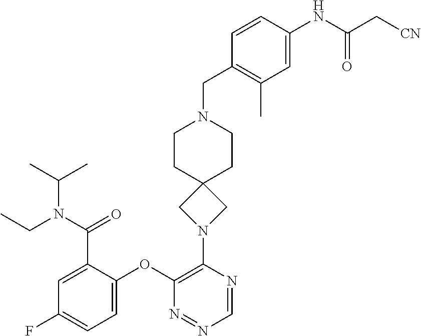 [SMILES: CCN(C(=O)c1cc(F)ccc1Oc1nncnc1N1CC2(CCN(Cc3ccc(NC(=O)CC#N)cc3C)CC2)C1)C(C)C] |
| 18 | 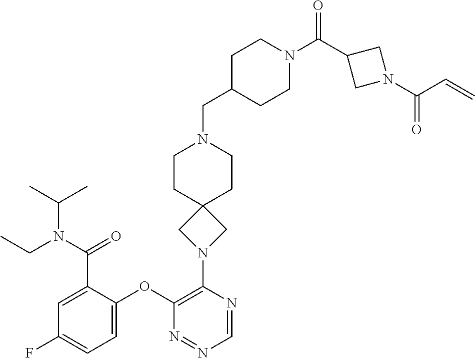 [SMILES: C=CC(=O)N1CC(C(=O)N2CCC(CN3CCC4(CC3)CN(c3ncnnc3Oc3ccc(F)cc3C(=O)N(CC)C(C)C)C4)CC2)C1] |
| 19 | 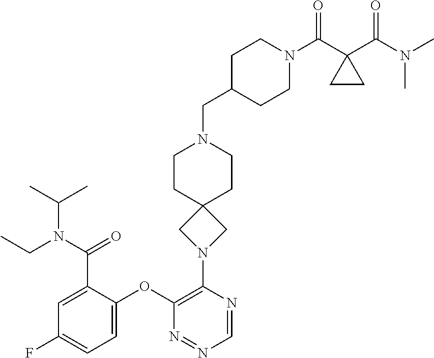 [SMILES: CCN(C(=O)c1cc(F)ccc1Oc1nncnc1N1CC2(CCN(CC3CCN(C(=O)C4(C(=O)N(C)C)CC4)CC3)CC2)C1)C(C)C] |
| 20 | 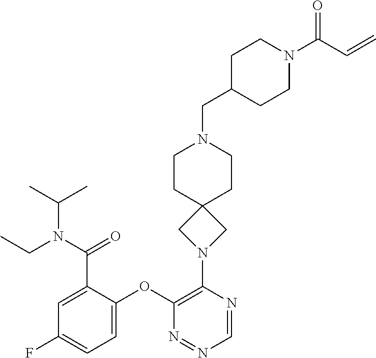 [SMILES: C=CC(=O)N1CCC(CN2CCC3(CC2)CN(c2ncnnc2Oc2ccc(F)cc2C(=O)N(CC)C(C)C)C3)CC1] |
| 21 | 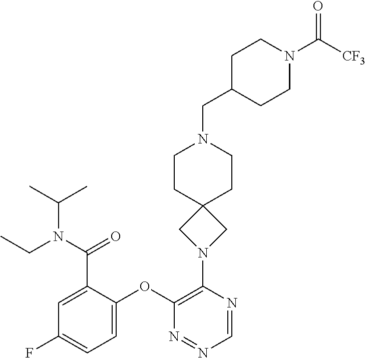 [SMILES: CCN(C(=O)c1cc(F)ccc1Oc1nncnc1N1CC2(CCN(CC3CCN(C(=O)C(F)(F)F)CC3)CC2)C1)C(C)C] |
| 22 | 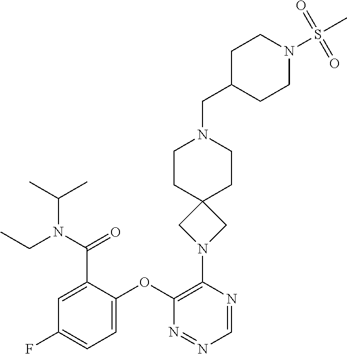 [SMILES: CCN(C(=O)c1cc(F)ccc1Oc1nncnc1N1CC2(CCN(CC3CCN(S(C)(=O)=O)CC3)CC2)C1)C(C)C] |
| 23 | 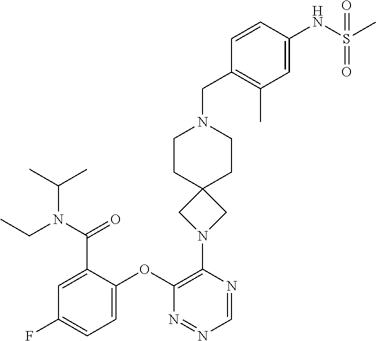 [SMILES: CCN(C(=O)c1cc(F)ccc1Oc1nncnc1N1CC2(CCN(Cc3ccc(NS(C)(=O)=O)cc3C)CC2)C1)C(C)C] |
| 24 | 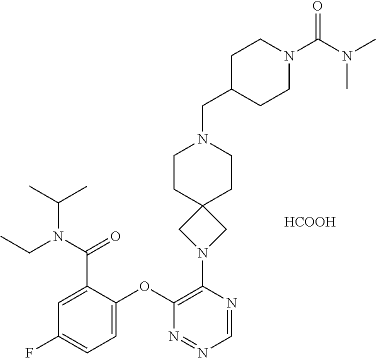 [SMILES: CCN(C(=O)c1cc(F)ccc1Oc1nncnc1N1CC2(CCN(CC3CCN(C(=O)N(C)C)CC3)CC2)C1)C(C)C ; "HCOOH"] |
| 25 | 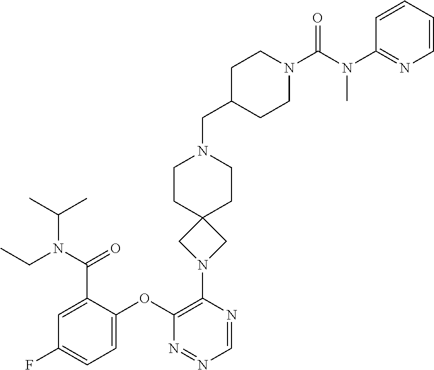 [SMILES: CCN(C(=O)c1cc(F)ccc1Oc1nncnc1N1CC2(CCN(CC3CCN(C(=O)N(C)c4ccccn4)CC3)CC2)C1)C(C)C] |
| 26 | 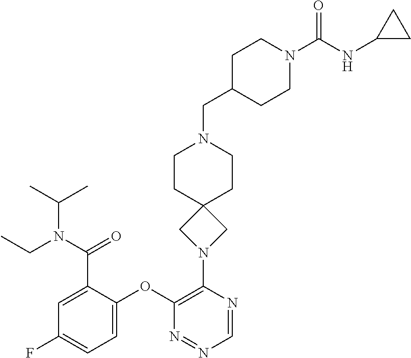 [SMILES: CCN(C(=O)c1cc(F)ccc1Oc1nncnc1N1CC2(CCN(CC3CCN(C(=O)NC4CC4)CC3)CC2)C1)C(C)C] |
| 27 | 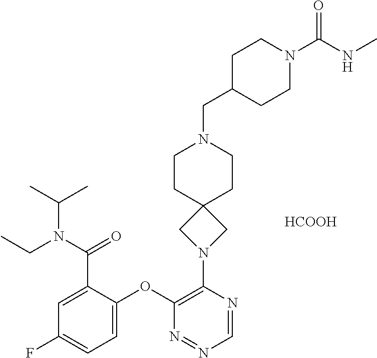 [SMILES: CCN(C(=O)c1cc(F)ccc1Oc1nncnc1N1CC2(CCN(CC3CCN(C(=O)NC)CC3)CC2)C1)C(C)C ; "HCOOH"] |
| 28 | 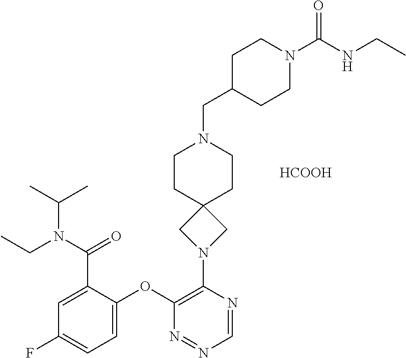 [SMILES: CCNC(=O)N1CCC(CN2CCC3(CC2)CN(c2ncnnc2Oc2ccc(F)cc2C(=O)N(CC)C(C)C)C3)CC1 ; "HCOOH"] |
| 29 | 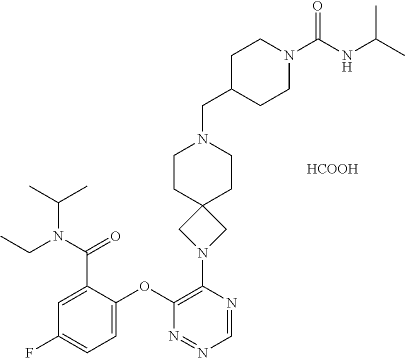 [SMILES: CCN(C(=O)c1cc(F)ccc1Oc1nncnc1N1CC2(CCN(CC3CCN(C(=O)NC(C)C)CC3)CC2)C1)C(C)C ; "HCOOH"] |
| 30 | 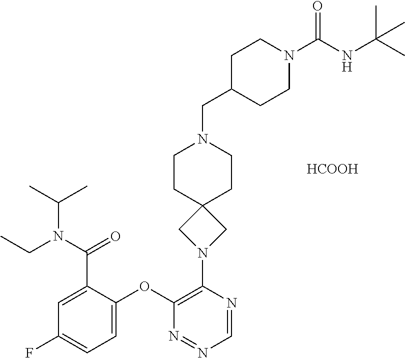 [SMILES: CCN(C(=O)c1cc(F)ccc1Oc1nncnc1N1CC2(CCN(CC3CCN(C(=O)NC(C)(C)C)CC3)CC2)C1)C(C)C ; "HCOOH"] |
| 31 | 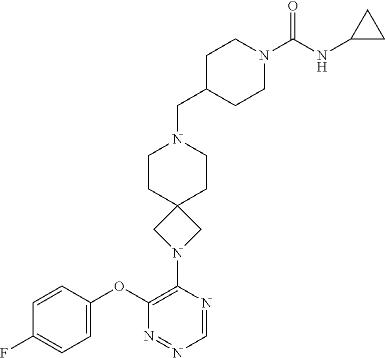 [SMILES: O=C(NC1CC1)N1CCC(CN2CCC3(CC2)CN(c2ncnnc2Oc2ccc(F)cc2)C3)CC1] |
| 32 | 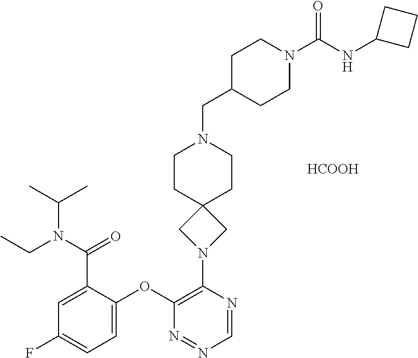 [SMILES: CCN(C(=O)c1cc(F)ccc1Oc1nncnc1N1CC2(CCN(CC3CCN(C(=O)NC4CCC4)CC3)CC2)C1)C(C)C ; "HCOOH"] |
| 33 | 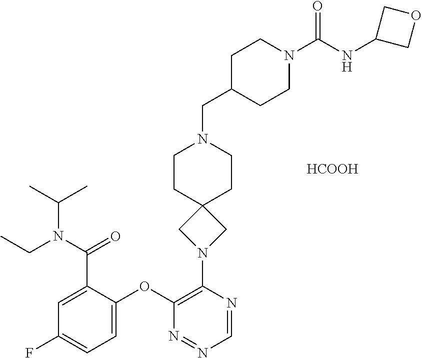 [SMILES: CCN(C(=O)c1cc(F)ccc1Oc1nncnc1N1CC2(CCN(CC3CCN(C(=O)NC4COC4)CC3)CC2)C1)C(C)C ; "HCOOH"] |
| 34 | 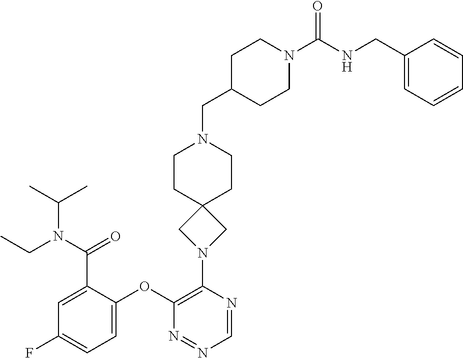 [SMILES: CCN(C(=O)c1cc(F)ccc1Oc1nncnc1N1CC2(CCN(CC3CCN(C(=O)NCc4ccccc4)CC3)CC2)C1)C(C)C] |
| 35 | 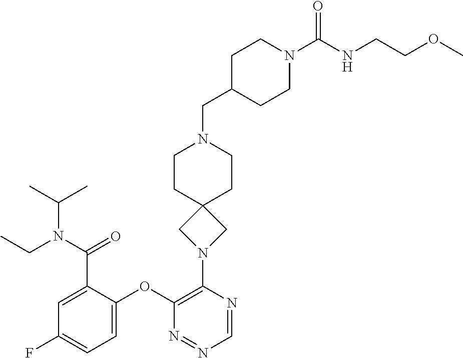 [SMILES: CCN(C(=O)c1cc(F)ccc1Oc1nncnc1N1CC2(CCN(CC3CCN(C(=O)NCCOC)CC3)CC2)C1)C(C)C] |
| 36 | 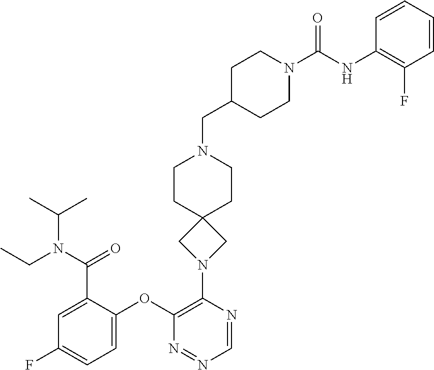 [SMILES: CCN(C(=O)c1cc(F)ccc1Oc1nncnc1N1CC2(CCN(CC3CCN(C(=O)Nc4ccccc4F)CC3)CC2)C1)C(C)C] |
| 37 | 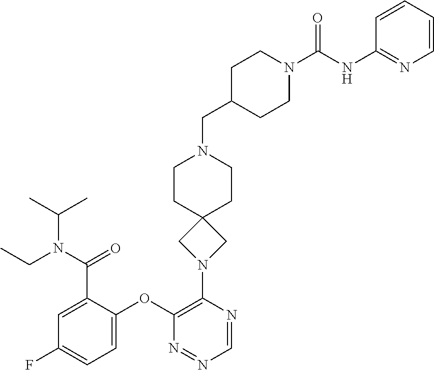 [SMILES: CCN(C(=O)c1cc(F)ccc1Oc1nncnc1N1CC2(CCN(CC3CCN(C(=O)Nc4ccccn4)CC3)CC2)C1)C(C)C] |
| 38 | 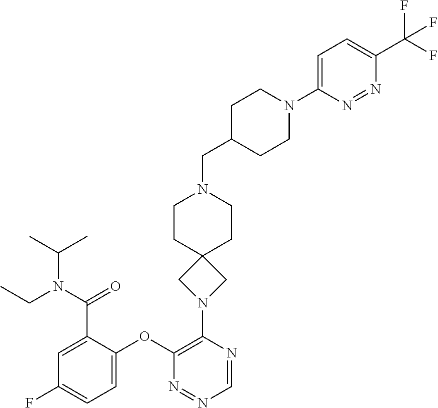 [SMILES: CCN(C(=O)c1cc(F)ccc1Oc1nncnc1N1CC2(CCN(CC3CCN(c4ccc(C(F)(F)F)nn4)CC3)CC2)C1)C(C)C] |
| 39 | 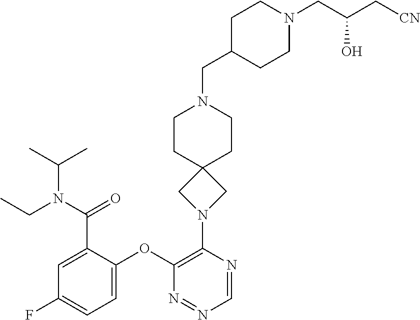 [SMILES: CCN(C(=O)c1cc(F)ccc1Oc1nncnc1N1CC2(CCN(CC3CCN(C[C@@H](O)CC#N)CC3)CC2)C1)C(C)C] |
| 40 | 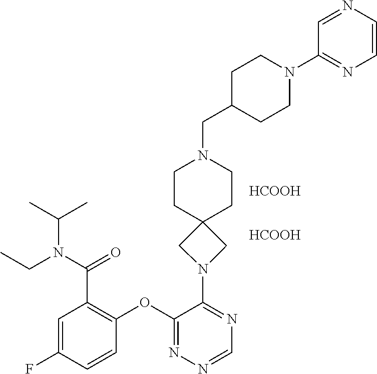 |
| 41 | 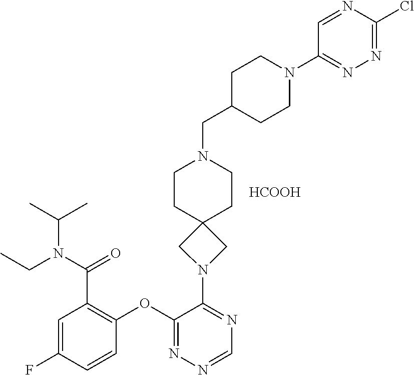 |
| 42 | 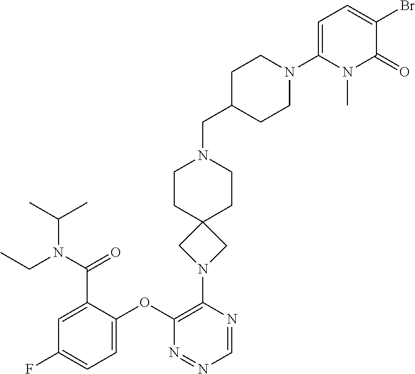 [SMILES: CCN(C(=O)c1cc(F)ccc1Oc1nncnc1N1CC2(CCN(CC3CCN(c4ccc(Br)c(=O)n4C)CC3)CC2)C1)C(C)C] |
| 43 | 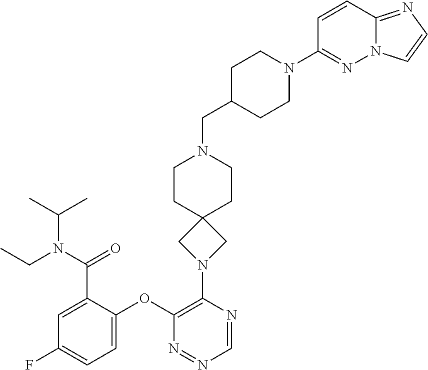 [SMILES: CCN(C(=O)c1cc(F)ccc1Oc1nncnc1N1CC2(CCN(CC3CCN(c4ccc5nccn5n4)CC3)CC2)C1)C(C)C] |
| 44 | 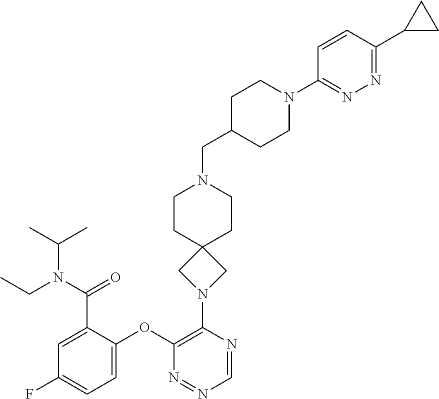 [SMILES: CCN(C(=O)c1cc(F)ccc1Oc1nncnc1N1CC2(CCN(CC3CCN(c4ccc(C5CC5)nn4)CC3)CC2)C1)C(C)C] |
| 45 | 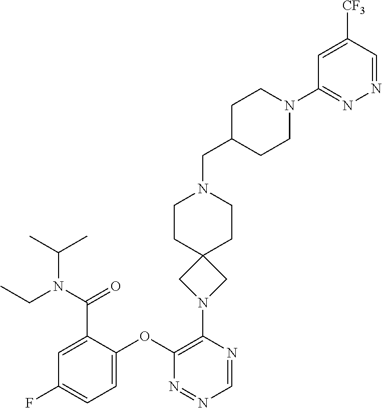 [SMILES: CCN(C(=O)c1cc(F)ccc1Oc1nncnc1N1CC2(CCN(CC3CCN(c4cc(C(F)(F)F)cnn4)CC3)CC2)C1)C(C)C] |
| 46 | 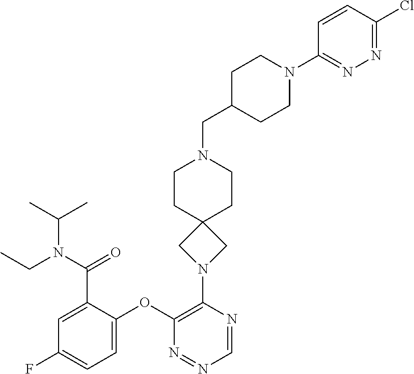 [SMILES: CCN(C(=O)c1cc(F)ccc1Oc1nncnc1N1CC2(CCN(CC3CCN(c4ccc(Cl)nn4)CC3)CC2)C1)C(C)C] |
| 47 | 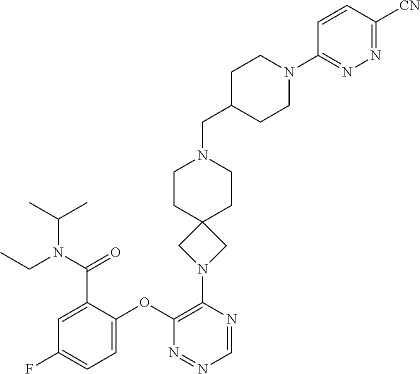 [SMILES: CCN(C(=O)c1cc(F)ccc1Oc1nncnc1N1CC2(CCN(CC3CCN(c4ccc(C#N)nn4)CC3)CC2)C1)C(C)C] |
| 48 | 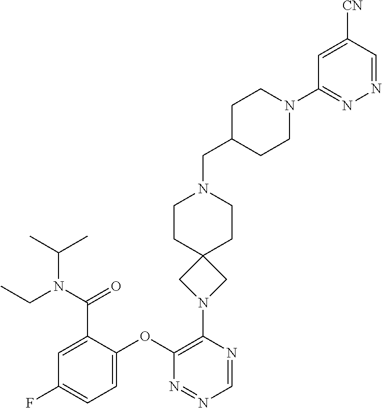 [SMILES: CCN(C(=O)c1cc(F)ccc1Oc1nncnc1N1CC2(CCN(CC3CCN(c4cc(C#N)cnn4)CC3)CC2)C1)C(C)C] |
| 49 | 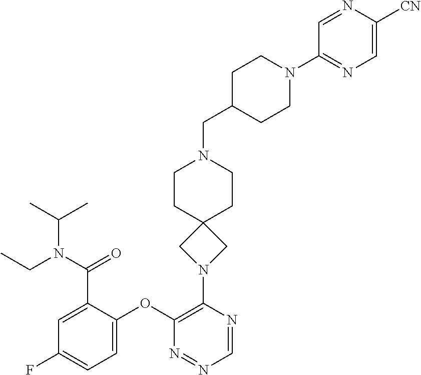 [SMILES: CCN(C(=O)c1cc(F)ccc1Oc1nncnc1N1CC2(CCN(CC3CCN(c4cnc(C#N)cn4)CC3)CC2)C1)C(C)C] |
| 50 | 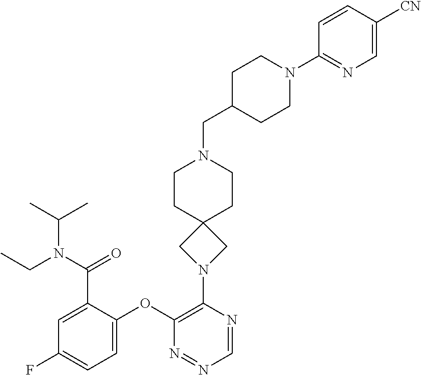 [SMILES: CCN(C(=O)c1cc(F)ccc1Oc1nncnc1N1CC2(CCN(CC3CCN(c4ccc(C#N)cn4)CC3)CC2)C1)C(C)C] |
| 51 | 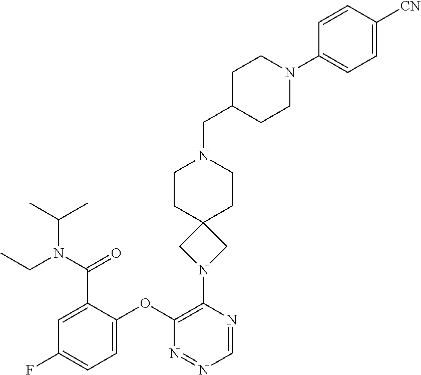 [SMILES: CCN(C(=O)c1cc(F)ccc1Oc1nncnc1N1CC2(CCN(CC3CCN(c4ccc(C#N)cc4)CC3)CC2)C1)C(C)C] |
| 52 | 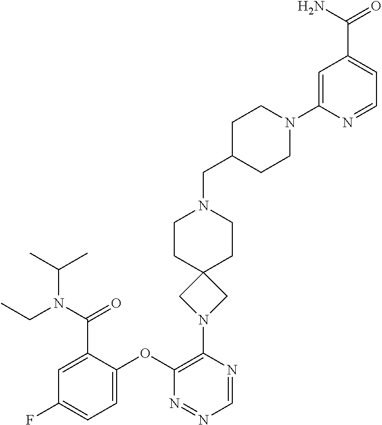 [SMILES: CCN(C(=O)c1cc(F)ccc1Oc1nncnc1N1CC2(CCN(CC3CCN(c4cc(C(N)=O)ccn4)CC3)CC2)C1)C(C)C] |
| 53 | 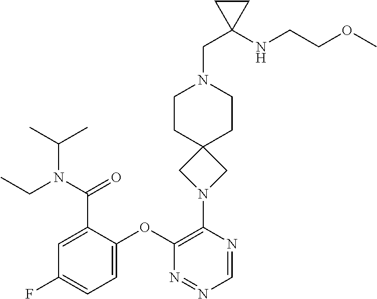 [SMILES: CCN(C(=O)c1cc(F)ccc1Oc1nncnc1N1CC2(CCN(CC3(NCCOC)CC3)CC2)C1)C(C)C] |
| 54 | 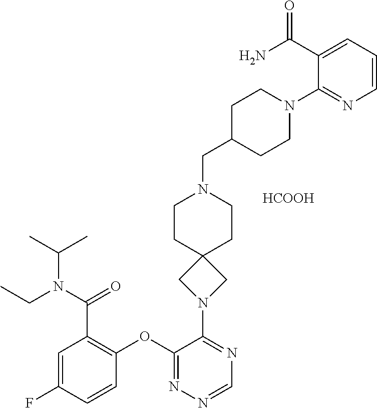 [SMILES: CCN(C(=O)c1cc(F)ccc1Oc1nncnc1N1CC2(CCN(CC3CCN(c4ncccc4C(N)=O)CC3)CC2)C1)C(C)C ; "HCOOH"] |
| 55 | 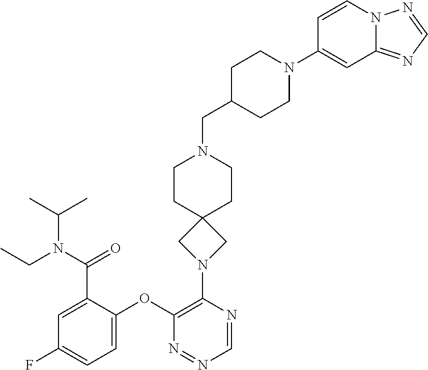 [SMILES: CCN(C(=O)c1cc(F)ccc1Oc1nncnc1N1CC2(CCN(CC3CCN(c4ccn5ncnc5c4)CC3)CC2)C1)C(C)C] |
| 56 | 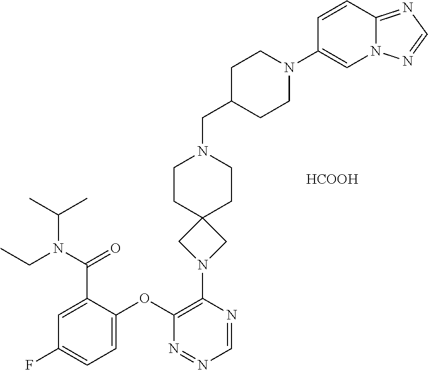 [SMILES: CCN(C(=O)c1cc(F)ccc1Oc1nncnc1N1CC2(CCN(CC3CCN(c4ccc5ncnn5c4)CC3)CC2)C1)C(C)C ; "HCOOH"] |
| 57 | 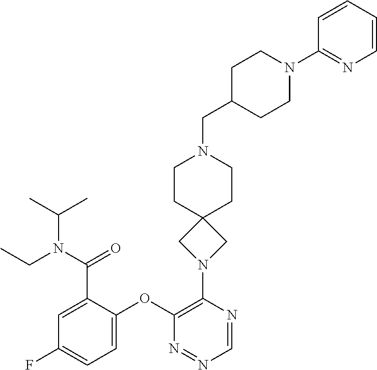 [SMILES: CCN(C(=O)c1cc(F)ccc1Oc1nncnc1N1CC2(CCN(CC3CCN(c4ccccn4)CC3)CC2)C1)C(C)C] |
| 58 | 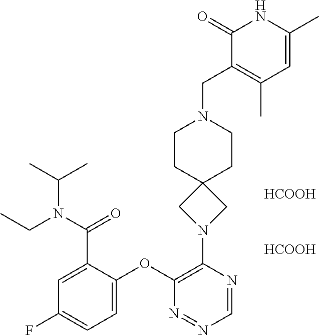 [SMILES: CCN(C(=O)c1cc(F)ccc1Oc1nncnc1N1CC2(CCN(Cc3c(C)cc(C)[nH]c3=O)CC2)C1)C(C)C ; "HCOOH" ; "HCOOH"] |
| 59 | 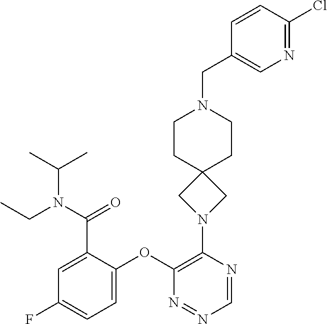 [SMILES: CCN(C(=O)c1cc(F)ccc1Oc1nncnc1N1CC2(CCN(Cc3ccc(Cl)nc3)CC2)C1)C(C)C] |
| 60 | 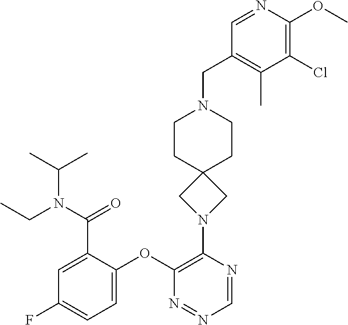 [SMILES: CCN(C(=O)c1cc(F)ccc1Oc1nncnc1N1CC2(CCN(Cc3cnc(OC)c(Cl)c3C)CC2)C1)C(C)C] |
| 61 | 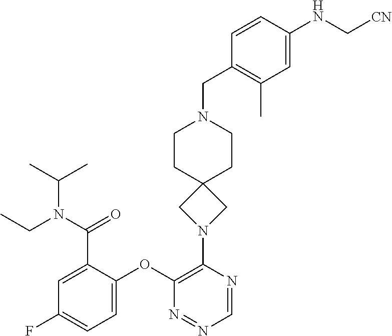 [SMILES: CCN(C(=O)c1cc(F)ccc1Oc1nncnc1N1CC2(CCN(Cc3ccc(NCC#N)cc3C)CC2)C1)C(C)C] |
| 62 | 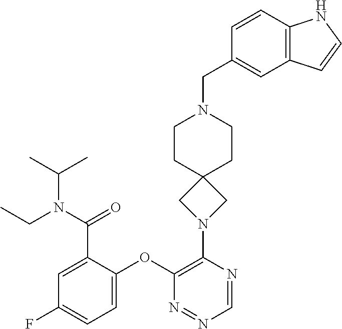 [SMILES: CCN(C(=O)c1cc(F)ccc1Oc1nncnc1N1CC2(CCN(Cc3ccc4[nH]ccc4c3)CC2)C1)C(C)C] |
| 63 | 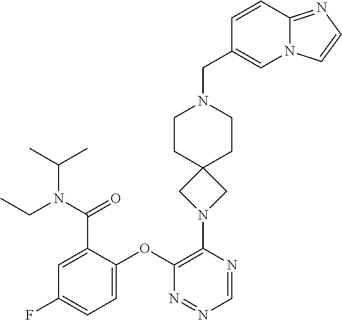 [SMILES: CCN(C(=O)c1cc(F)ccc1Oc1nncnc1N1CC2(CCN(Cc3ccc4nccn4c3)CC2)C1)C(C)C] |
| 64 | 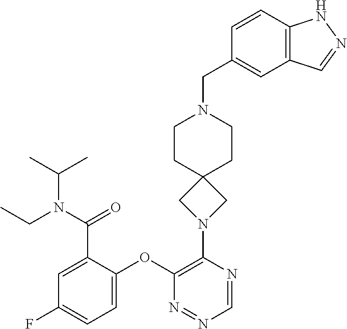 [SMILES: CCN(C(=O)c1cc(F)ccc1Oc1nncnc1N1CC2(CCN(Cc3ccc4[nH]ncc4c3)CC2)C1)C(C)C] |
| 65 | 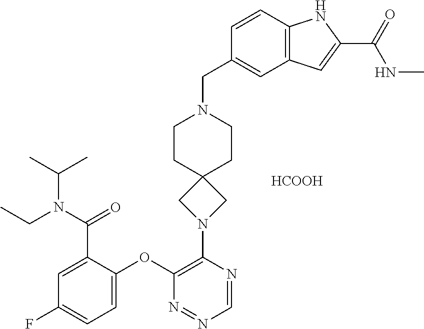 [SMILES: CCN(C(=O)c1cc(F)ccc1Oc1nncnc1N1CC2(CCN(Cc3ccc4[nH]c(C(=O)NC)cc4c3)CC2)C1)C(C)C ; "HCOOH"] |
| 66 | 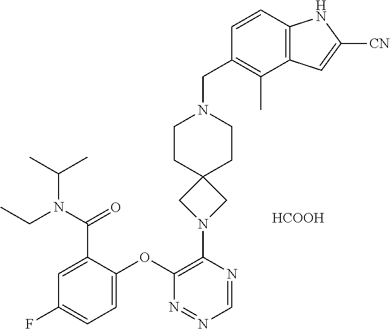 [SMILES: CCN(C(=O)c1cc(F)ccc1Oc1nncnc1N1CC2(CCN(Cc3ccc4[nH]c(C#N)cc4c3C)CC2)C1)C(C)C ; "HCOOH"] |
| 67 | 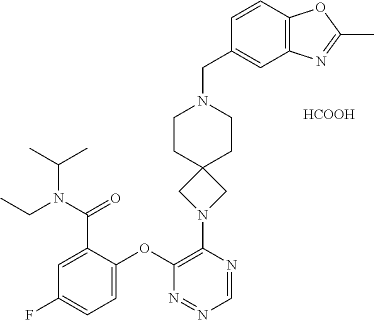 [SMILES: CCN(C(=O)c1cc(F)ccc1Oc1nncnc1N1CC2(CCN(Cc3ccc4oc(C)nc4c3)CC2)C1)C(C)C ; "HCOOH"] |
| 68 | 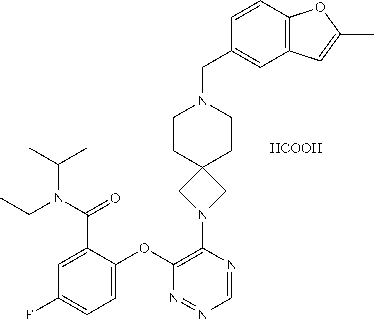 [SMILES: CCN(C(=O)c1cc(F)ccc1Oc1nncnc1N1CC2(CCN(Cc3ccc4oc(C)cc4c3)CC2)C1)C(C)C ; "HCOOH"] |
| 69 | 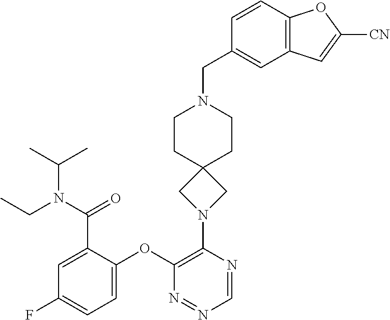 [SMILES: CCN(C(=O)c1cc(F)ccc1Oc1nncnc1N1CC2(CCN(Cc3ccc4oc(C#N)cc4c3)CC2)C1)C(C)C] |
| 70 | 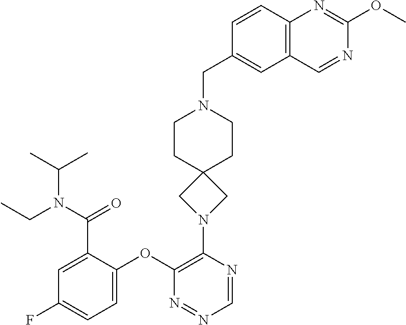 [SMILES: CCN(C(=O)c1cc(F)ccc1Oc1nncnc1N1CC2(CCN(Cc3ccc4nc(OC)ncc4c3)CC2)C1)C(C)C] |
| 71 | 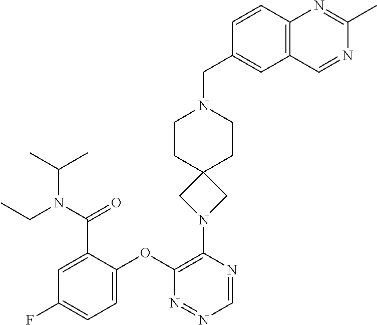 [SMILES: CCN(C(=O)c1cc(F)ccc1Oc1nncnc1N1CC2(CCN(Cc3ccc4nc(C)ncc4c3)CC2)C1)C(C)C] |
| 72 | 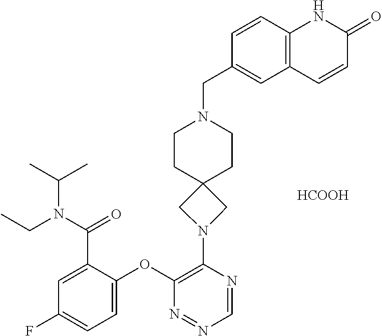 [SMILES: CCN(C(=O)c1cc(F)ccc1Oc1nncnc1N1CC2(CCN(Cc3ccc4[nH]c(=O)ccc4c3)CC2)C1)C(C)C ; "HCOOH"] |
| 73 | 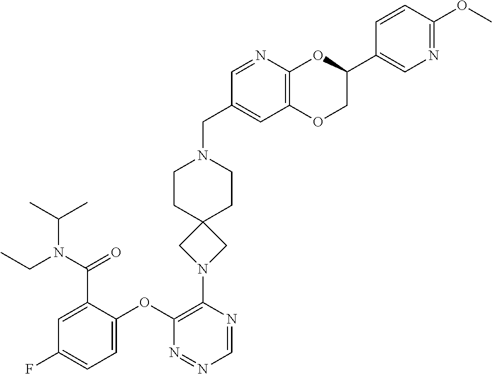 [SMILES: CCN(C(=O)c1cc(F)ccc1Oc1nncnc1N1CC2(CCN(Cc3cnc4c(c3)OC[C@H](c3ccc(OC)nc3)O4)CC2)C1)C(C)C] |
| 74 | 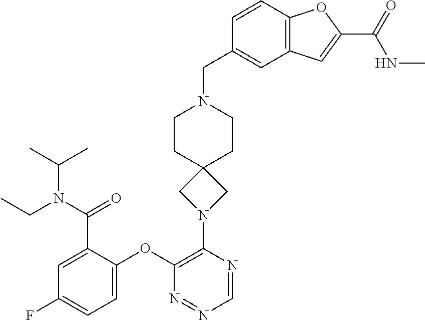 [SMILES: CCN(C(=O)c1cc(F)ccc1Oc1nncnc1N1CC2(CCN(Cc3ccc4oc(C(=O)NC)cc4c3)CC2)C1)C(C)C] |
| 75 | 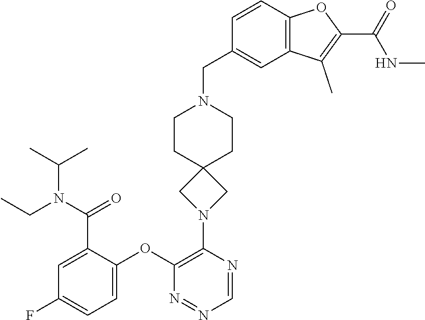 [SMILES: CCN(C(=O)c1cc(F)ccc1Oc1nncnc1N1CC2(CCN(Cc3ccc4oc(C(=O)NC)c(C)c4c3)CC2)C1)C(C)C] |
| 76 | 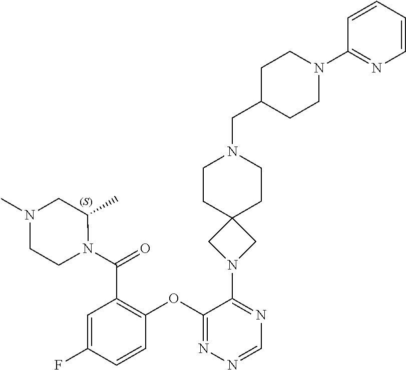 |
| 77 | 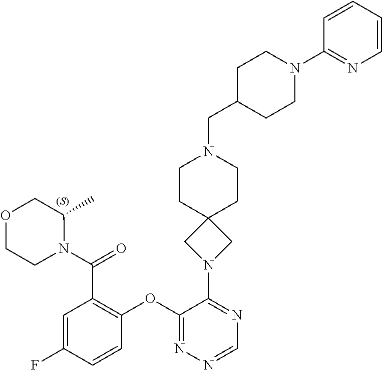 |
| 78 | 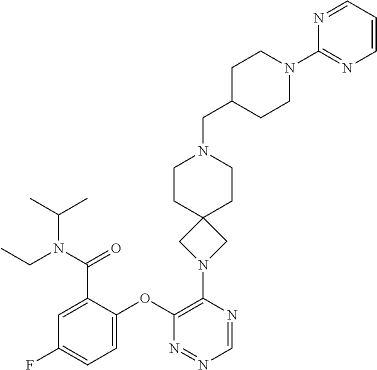 [SMILES: CCN(C(=O)c1cc(F)ccc1Oc1nncnc1N1CC2(CCN(CC3CCN(c4ncccn4)CC3)CC2)C1)C(C)C] |
| 79 | 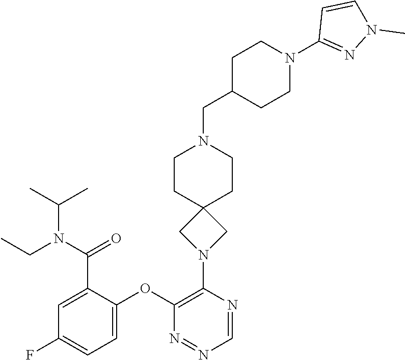 [SMILES: CCN(C(=O)c1cc(F)ccc1Oc1nncnc1N1CC2(CCN(CC3CCN(c4ccn(C)n4)CC3)CC2)C1)C(C)C] |
| 80 | 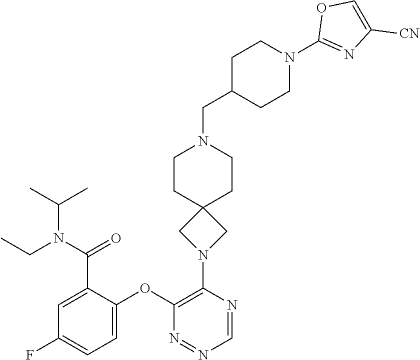 [SMILES: CCN(C(=O)c1cc(F)ccc1Oc1nncnc1N1CC2(CCN(CC3CCN(c4nc(C#N)co4)CC3)CC2)C1)C(C)C] |
| 81 | 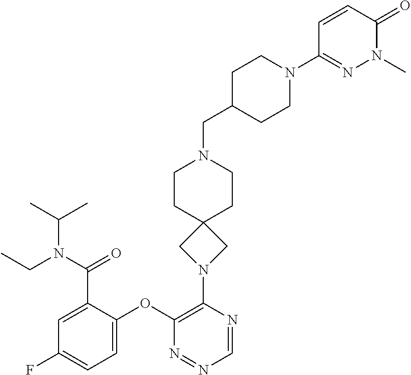 [SMILES: CCN(C(=O)c1cc(F)ccc1Oc1nncnc1N1CC2(CCN(CC3CCN(c4ccc(=O)n(C)n4)CC3)CC2)C1)C(C)C] |
| 82 | 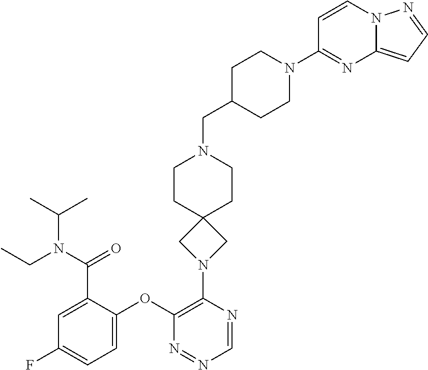 [SMILES: CCN(C(=O)c1cc(F)ccc1Oc1nncnc1N1CC2(CCN(CC3CCN(c4ccn5nccc5n4)CC3)CC2)C1)C(C)C] |
| 83 | 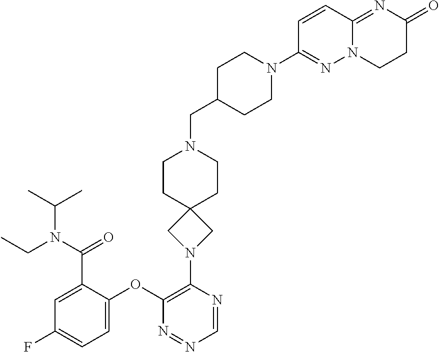 [SMILES: CCN(C(=O)c1cc(F)ccc1Oc1nncnc1N1CC2(CCN(CC3CCN(C4=NN5CCC(=O)N=C5C=C4)CC3)CC2)C1)C(C)C] |
| 84 | 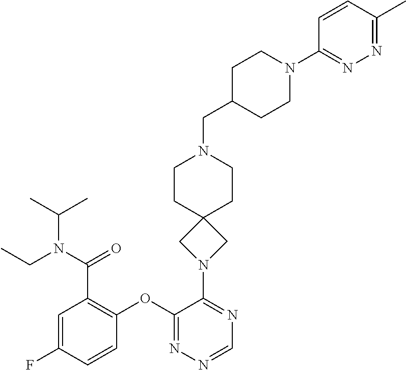 [SMILES: CCN(C(=O)c1cc(F)ccc1Oc1nncnc1N1CC2(CCN(CC3CCN(c4ccc(C)nn4)CC3)CC2)C1)C(C)C] |
| 85 | 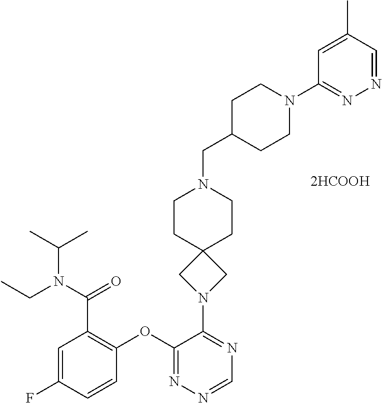 [SMILES: CCN(C(=O)c1cc(F)ccc1Oc1nncnc1N1CC2(CCN(CC3CCN(c4cc(C)cnn4)CC3)CC2)C1)C(C)C ; "2HCOOH"] |
| 86 | 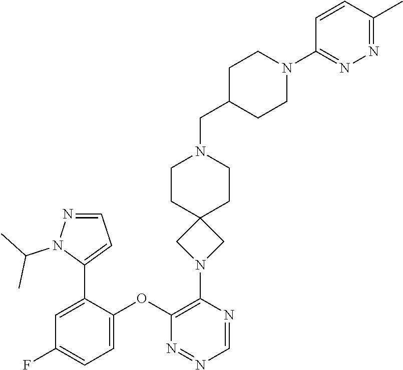 [SMILES: Cc1ccc(N2CCC(CN3CCC4(CC3)CN(c3ncnnc3Oc3ccc(F)cc3-c3ccnn3C(C)C)C4)CC2)nn1] |
| 87 | 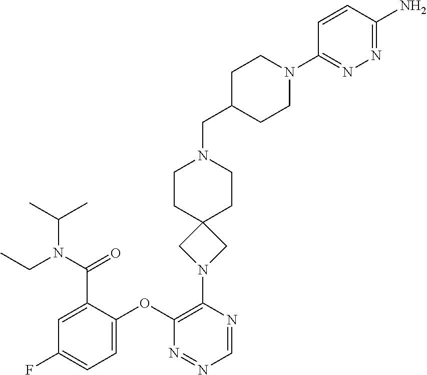 [SMILES: CCN(C(=O)c1cc(F)ccc1Oc1nncnc1N1CC2(CCN(CC3CCN(c4ccc(N)nn4)CC3)CC2)C1)C(C)C] |
| 88 | 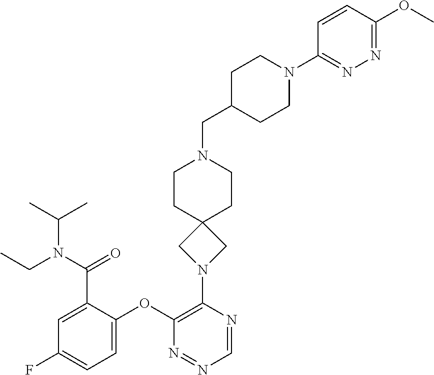 [SMILES: CCN(C(=O)c1cc(F)ccc1Oc1nncnc1N1CC2(CCN(CC3CCN(c4ccc(OC)nn4)CC3)CC2)C1)C(C)C] |
| 89 | 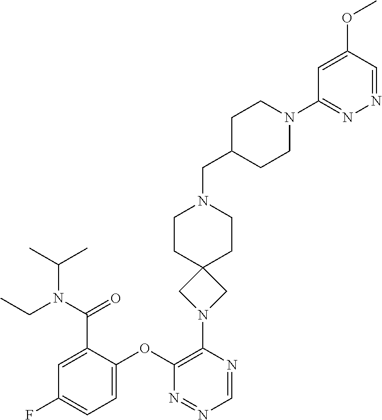 [SMILES: CCN(C(=O)c1cc(F)ccc1Oc1nncnc1N1CC2(CCN(CC3CCN(c4cc(OC)cnn4)CC3)CC2)C1)C(C)C] |
| 90 | 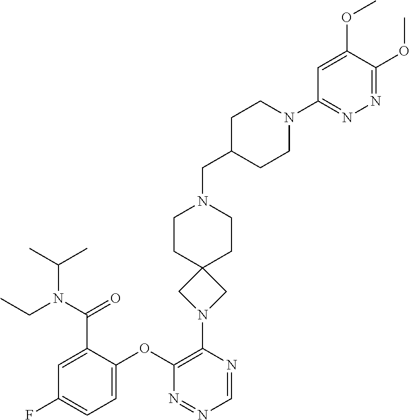 [SMILES: CCN(C(=O)c1cc(F)ccc1Oc1nncnc1N1CC2(CCN(CC3CCN(c4cc(OC)c(OC)nn4)CC3)CC2)C1)C(C)C] |
| 91 | 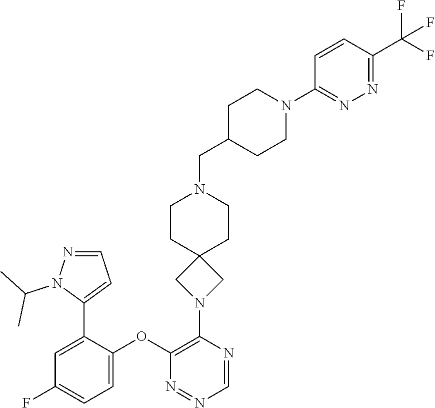 [SMILES: CC(C)n1nccc1-c1cc(F)ccc1Oc1nncnc1N1CC2(CCN(CC3CCN(c4ccc(C(F)(F)F)nn4)CC3)CC2)C1] |
| 92 | 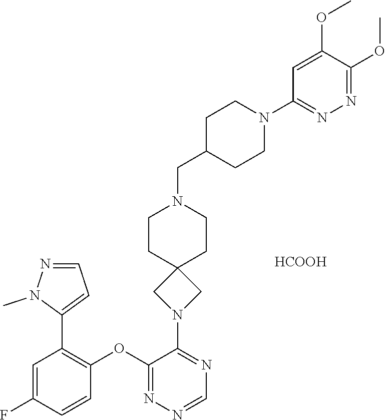 |
| 93 | 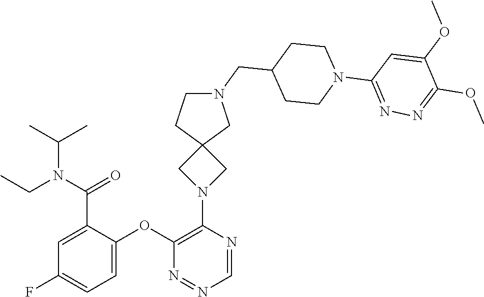 [SMILES: CCN(C(=O)c1cc(F)ccc1Oc1nncnc1N1CC2(CCN(CC3CCN(c4cc(OC)c(OC)nn4)CC3)C2)C1)C(C)C] |
| 94 | 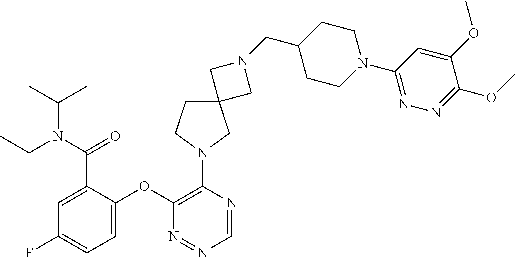 [SMILES: CCN(C(=O)c1cc(F)ccc1Oc1nncnc1N1CCC2(CN(CC3CCN(c4cc(OC)c(OC)nn4)CC3)C2)C1)C(C)C] |
| 95 | 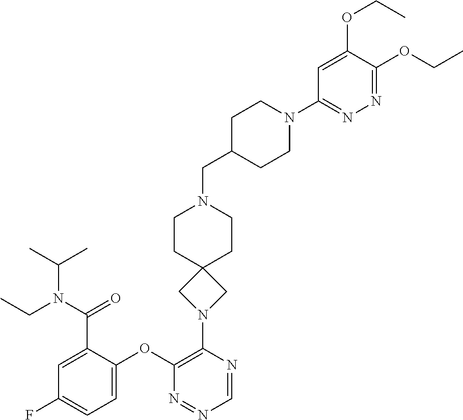 [SMILES: CCOc1cc(N2CCC(CN3CCC4(CC3)CN(c3ncnnc3Oc3ccc(F)cc3C(=O)N(CC)C(C)C)C4)CC2)nnc1OCC] |
| 96 | 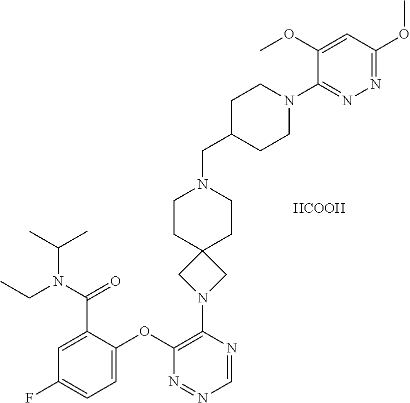 [SMILES: CCN(C(=O)c1cc(F)ccc1Oc1nncnc1N1CC2(CCN(CC3CCN(c4nnc(OC)cc4OC)CC3)CC2)C1)C(C)C ; "HCOOH"] |
| 97 | 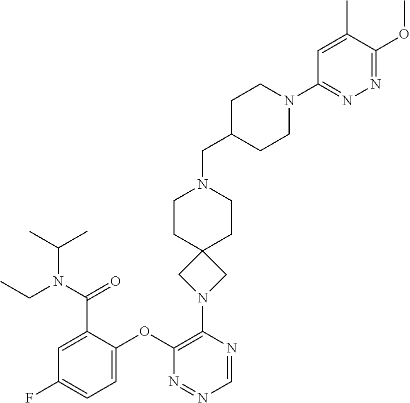 [SMILES: CCN(C(=O)c1cc(F)ccc1Oc1nncnc1N1CC2(CCN(CC3CCN(c4cc(C)c(OC)nn4)CC3)CC2)C1)C(C)C] |
| 98 | 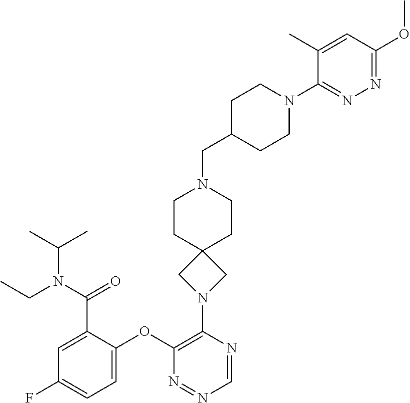 [SMILES: CCN(C(=O)c1cc(F)ccc1Oc1nncnc1N1CC2(CCN(CC3CCN(c4nnc(OC)cc4C)CC3)CC2)C1)C(C)C] |
| 99 | 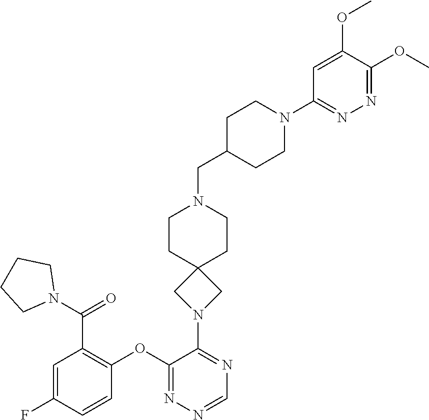 [SMILES: COc1cc(N2CCC(CN3CCC4(CC3)CN(c3ncnnc3Oc3ccc(F)cc3C(=O)N3CCCC3)C4)CC2)nnc1OC] |
| 100 | 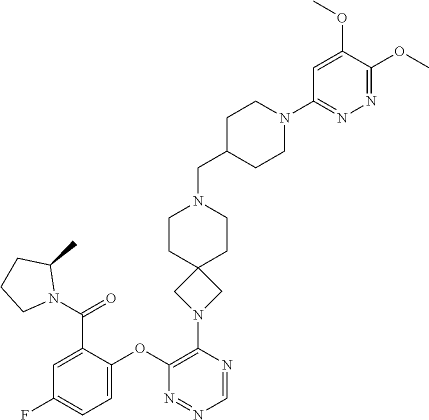 [SMILES: COc1cc(N2CCC(CN3CCC4(CC3)CN(c3ncnnc3Oc3ccc(F)cc3C(=O)N3CCC[C@H]3C)C4)CC2)nnc1OC] |
| 101 | 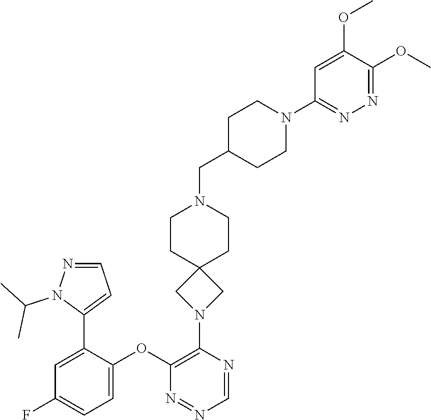 [SMILES: COc1cc(N2CCC(CN3CCC4(CC3)CN(c3ncnnc3Oc3ccc(F)cc3-c3ccnn3C(C)C)C4)CC2)nnc1OC] |
| 102 | 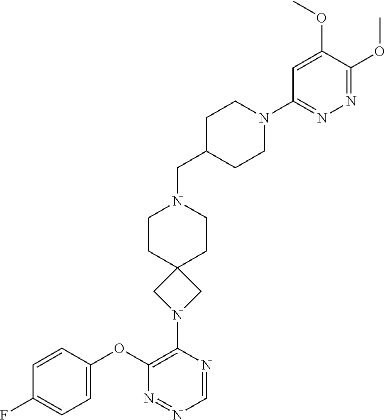 [SMILES: COc1cc(N2CCC(CN3CCC4(CC3)CN(c3ncnnc3Oc3ccc(F)cc3)C4)CC2)nnc1OC] |
| 103 | 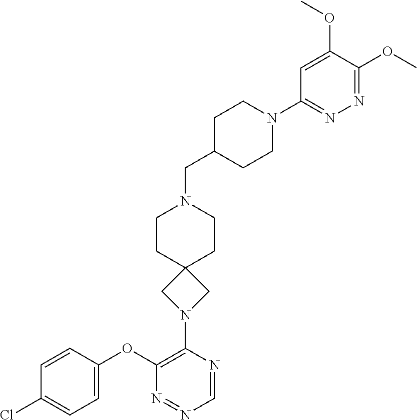 [SMILES: COc1cc(N2CCC(CN3CCC4(CC3)CN(c3ncnnc3Oc3ccc(Cl)cc3)C4)CC2)nnc1OC] |
| 104 | 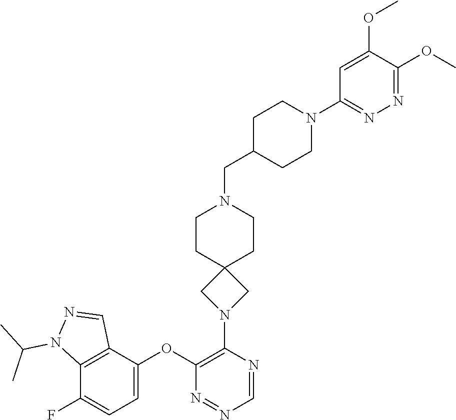 [SMILES: COc1cc(N2CCC(CN3CCC4(CC3)CN(c3ncnnc3Oc3ccc(F)c5c3cnn5C(C)C)C4)CC2)nnc1OC] |
| 105 | 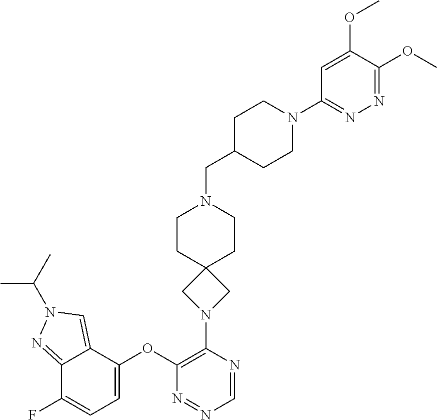 [SMILES: COc1cc(N2CCC(CN3CCC4(CC3)CN(c3ncnnc3Oc3ccc(F)c5nn(C(C)C)cc35)C4)CC2)nnc1OC] |
| 106 | 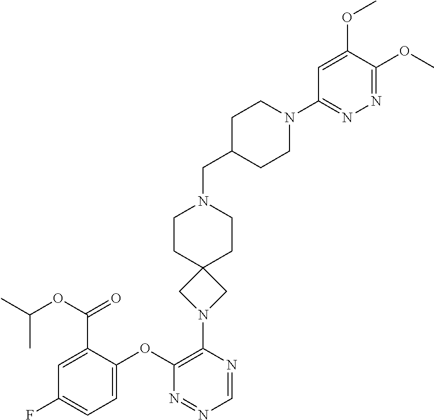 [SMILES: COc1cc(N2CCC(CN3CCC4(CC3)CN(c3ncnnc3Oc3ccc(F)cc3C(=O)OC(C)C)C4)CC2)nnc1OC] |
| 107 | 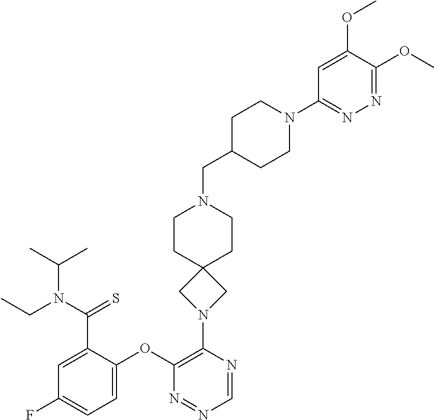 [SMILES: CCN(C(=S)c1cc(F)ccc1Oc1nncnc1N1CC2(CCN(CC3CCN(c4cc(OC)c(OC)nn4)CC3)CC2)C1)C(C)C] |
| 108 | 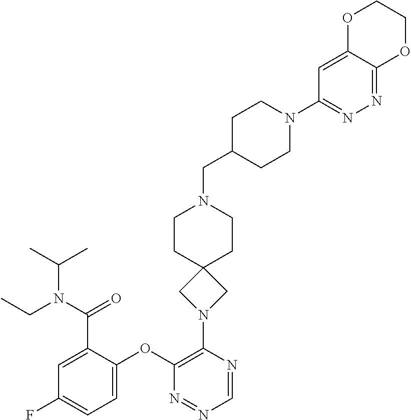 [SMILES: CCN(C(=O)c1cc(F)ccc1Oc1nncnc1N1CC2(CCN(CC3CCN(c4cc5c(nn4)OCCO5)CC3)CC2)C1)C(C)C] |
| 109 | 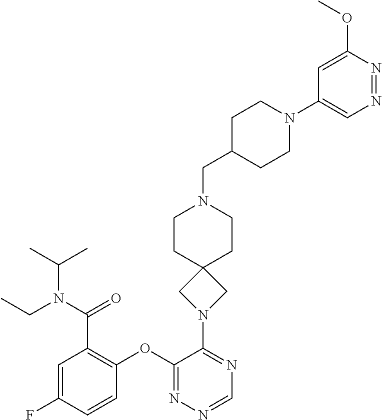 [SMILES: CCN(C(=O)c1cc(F)ccc1Oc1nncnc1N1CC2(CCN(CC3CCN(c4cnnc(OC)c4)CC3)CC2)C1)C(C)C] |
| 110 | 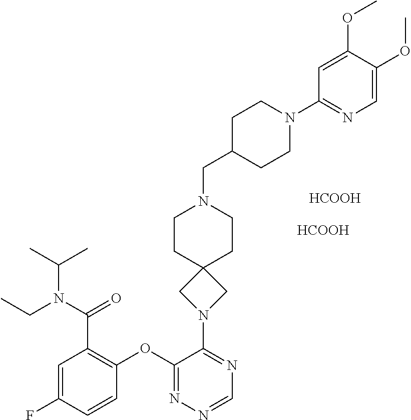 [SMILES: CCN(C(=O)c1cc(F)ccc1Oc1nncnc1N1CC2(CCN(CC3CCN(c4cc(OC)c(OC)cn4)CC3)CC2)C1)C(C)C ; "HCOOH" ; "HCOOH"] |
| 111 | 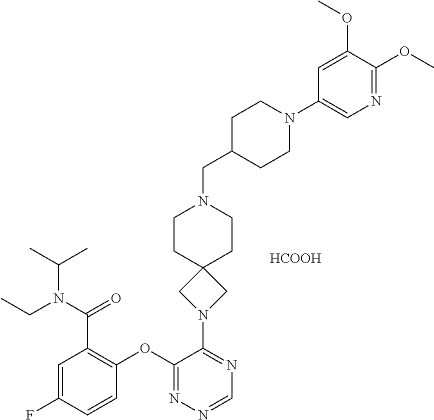 [SMILES: CCN(C(=O)c1cc(F)ccc1Oc1nncnc1N1CC2(CCN(CC3CCN(c4cnc(OC)c(OC)c4)CC3)CC2)C1)C(C)C ; "HCOOH"] |
| 112 | 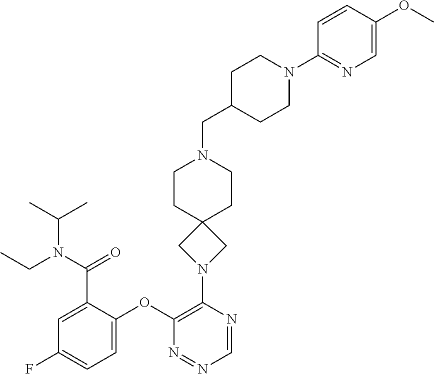 [SMILES: CCN(C(=O)c1cc(F)ccc1Oc1nncnc1N1CC2(CCN(CC3CCN(c4ccc(OC)cn4)CC3)CC2)C1)C(C)C] |
| 113 | 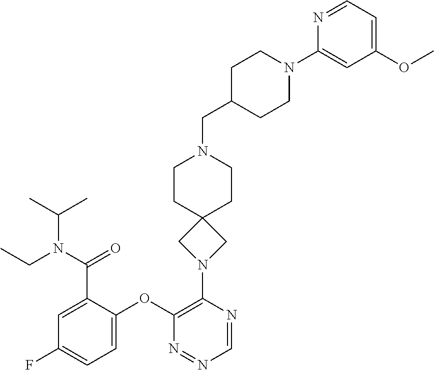 [SMILES: CCN(C(=O)c1cc(F)ccc1Oc1nncnc1N1CC2(CCN(CC3CCN(c4cc(OC)ccn4)CC3)CC2)C1)C(C)C] |
| 114 | 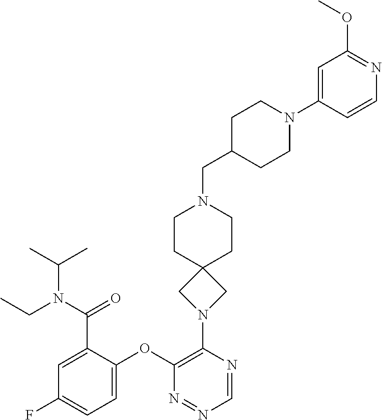 [SMILES: CCN(C(=O)c1cc(F)ccc1Oc1nncnc1N1CC2(CCN(CC3CCN(c4ccnc(OC)c4)CC3)CC2)C1)C(C)C] |
| 115 | 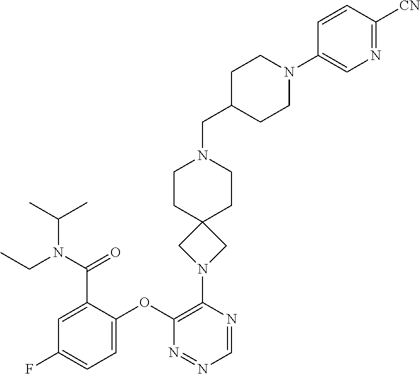 [SMILES: CCN(C(=O)c1cc(F)ccc1Oc1nncnc1N1CC2(CCN(CC3CCN(c4ccc(C#N)nc4)CC3)CC2)C1)C(C)C] |
| 116 | 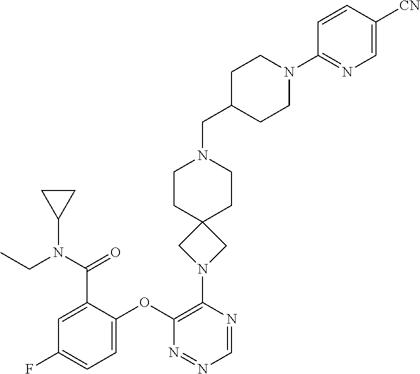 [SMILES: CCN(C(=O)c1cc(F)ccc1Oc1nncnc1N1CC2(CCN(CC3CCN(c4ccc(C#N)cn4)CC3)CC2)C1)C1CC1] |
| 117 | 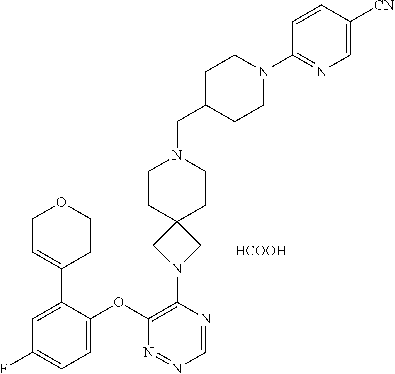 [SMILES: N#Cc1ccc(N2CCC(CN3CCC4(CC3)CN(c3ncnnc3Oc3ccc(F)cc3C3=CCOCC3)C4)CC2)nc1 ; "HCOOH"] |
| 118 | 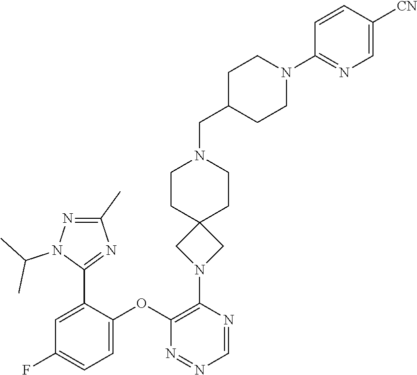 [SMILES: Cc1nc(-c2cc(F)ccc2Oc2nncnc2N2CC3(CCN(CC4CCN(c5ccc(C#N)cn5)CC4)CC3)C2)n(C(C)C)n1] |
| 119 | 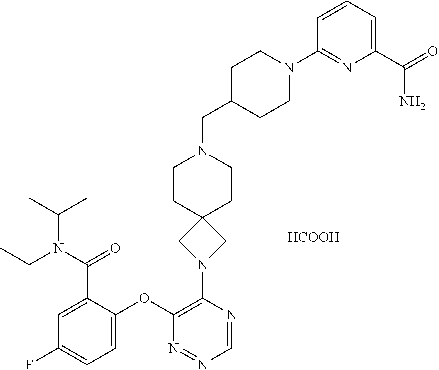 [SMILES: CCN(C(=O)c1cc(F)ccc1Oc1nncnc1N1CC2(CCN(CC3CCN(c4cccc(C(N)=O)n4)CC3)CC2)C1)C(C)C ; "HCOOH"] |
| 120 | 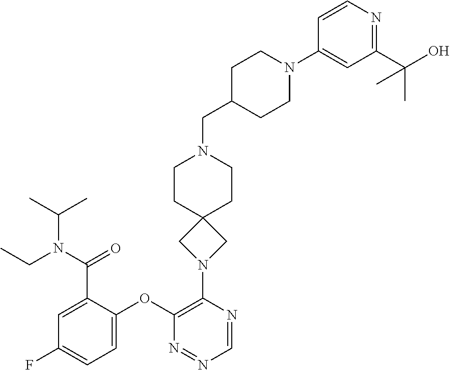 [SMILES: CCN(C(=O)c1cc(F)ccc1Oc1nncnc1N1CC2(CCN(CC3CCN(c4ccnc(C(C)(C)O)c4)CC3)CC2)C1)C(C)C] |
| 121 | 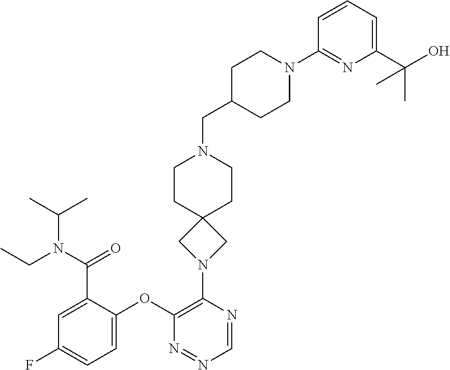 [SMILES: CCN(C(=O)c1cc(F)ccc1Oc1nncnc1N1CC2(CCN(CC3CCN(c4cccc(C(C)(C)O)n4)CC3)CC2)C1)C(C)C] |
| 122 | 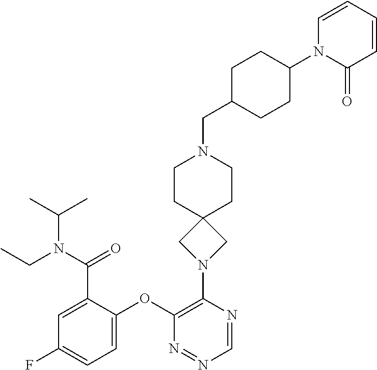 [SMILES: CCN(C(=O)c1cc(F)ccc1Oc1nncnc1N1CC2(CCN(CC3CCC(n4ccccc4=O)CC3)CC2)C1)C(C)C] |
| 123 | 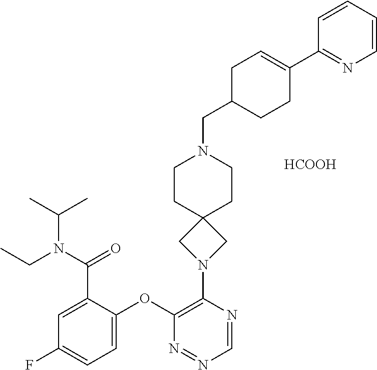 [SMILES: CCN(C(=O)c1cc(F)ccc1Oc1nncnc1N1CC2(CCN(CC3CC=C(c4ccccn4)CC3)CC2)C1)C(C)C ; "HCOOH"] |
| 124 | 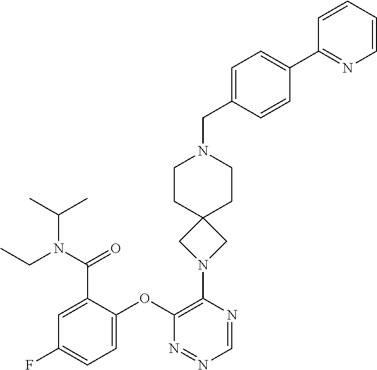 [SMILES: CCN(C(=O)c1cc(F)ccc1Oc1nncnc1N1CC2(CCN(Cc3ccc(-c4ccccn4)cc3)CC2)C1)C(C)C] |
| 125 | 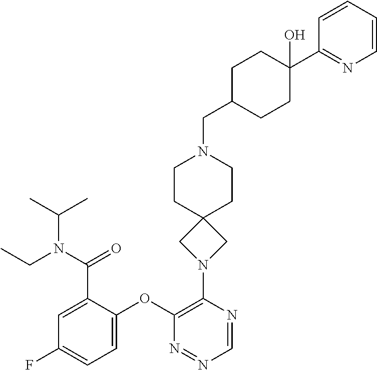 [SMILES: CCN(C(=O)c1cc(F)ccc1Oc1nncnc1N1CC2(CCN(CC3CCC(O)(c4ccccn4)CC3)CC2)C1)C(C)C] |
| 126 | 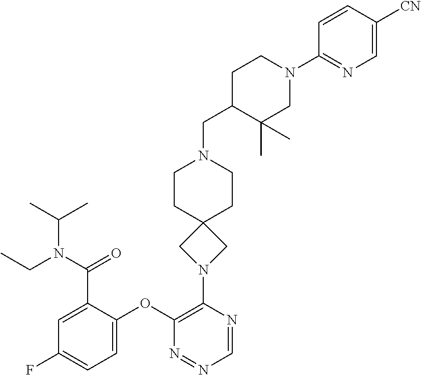 [SMILES: CCN(C(=O)c1cc(F)ccc1Oc1nncnc1N1CC2(CCN(CC3CCN(c4ccc(C#N)cn4)CC3(C)C)CC2)C1)C(C)C] |
| 127 | 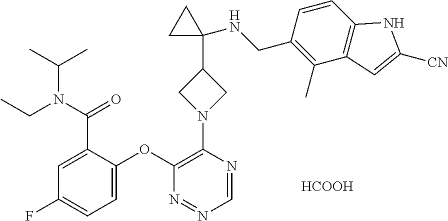 [SMILES: CCN(C(=O)c1cc(F)ccc1Oc1nncnc1N1CC(C2(NCc3ccc4[nH]c(C#N)cc4c3C)CC2)C1)C(C)C ; "HCOOH"] |
| 128 | 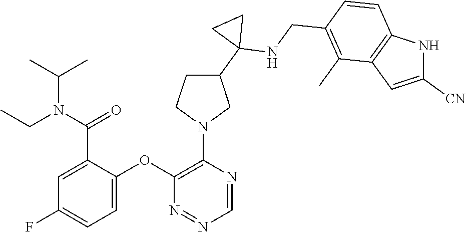 [SMILES: CCN(C(=O)c1cc(F)ccc1Oc1nncnc1N1CCC(C2(NCc3ccc4[nH]c(C#N)cc4c3C)CC2)C1)C(C)C] |
| 129 | 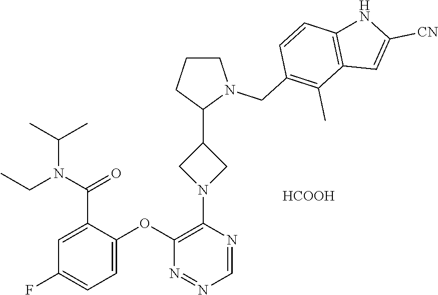 [SMILES: CCN(C(=O)c1cc(F)ccc1Oc1nncnc1N1CC(C2CCCN2Cc2ccc3[nH]c(C#N)cc3c2C)C1)C(C)C ; "HCOOH"] |
| 130 | 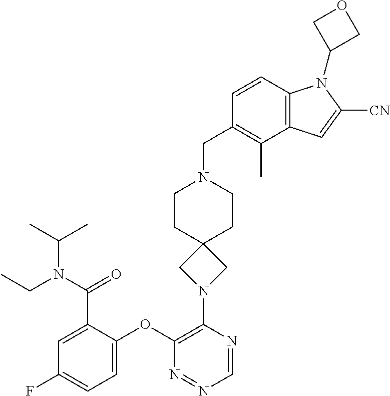 [SMILES: CCN(C(=O)c1cc(F)ccc1Oc1nncnc1N1CC2(CCN(Cc3ccc4c(cc(C#N)n4C4COC4)c3C)CC2)C1)C(C)C] |
| 131 | 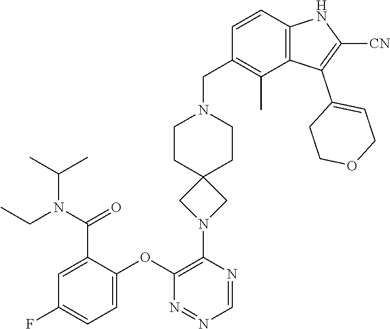 [SMILES: CCN(C(=O)c1cc(F)ccc1Oc1nncnc1N1CC2(CCN(Cc3ccc4[nH]c(C#N)c(C5=CCOCC5)c4c3C)CC2)C1)C(C)C] |
| 132 | 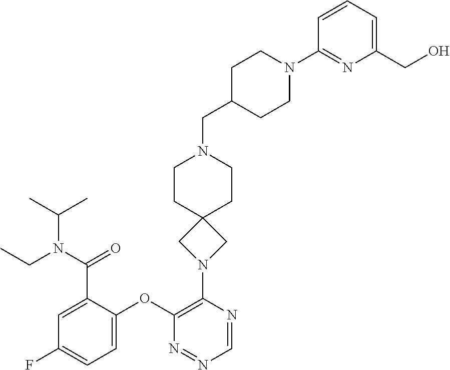 [SMILES: CCN(C(=O)c1cc(F)ccc1Oc1nncnc1N1CC2(CCN(CC3CCN(c4cccc(CO)n4)CC3)CC2)C1)C(C)C] |
| 133 | 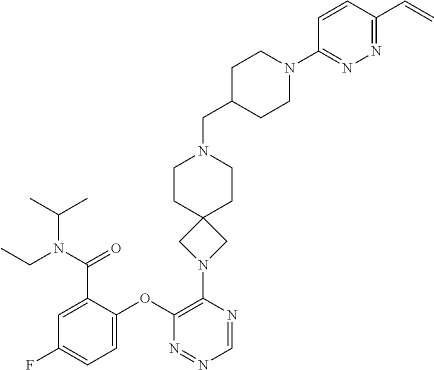 [SMILES: C=Cc1ccc(N2CCC(CN3CCC4(CC3)CN(c3ncnnc3Oc3ccc(F)cc3C(=O)N(CC)C(C)C)C4)CC2)nn1] |
| 134 | 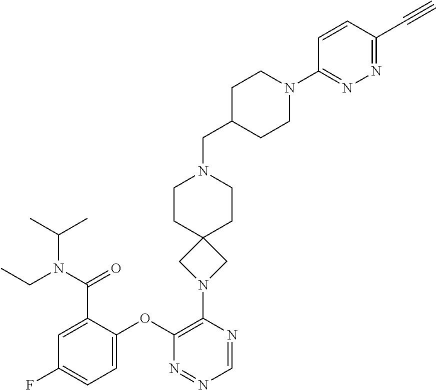 [SMILES: C#Cc1ccc(N2CCC(CN3CCC4(CC3)CN(c3ncnnc3Oc3ccc(F)cc3C(=O)N(CC)C(C)C)C4)CC2)nn1] |
| 135 | 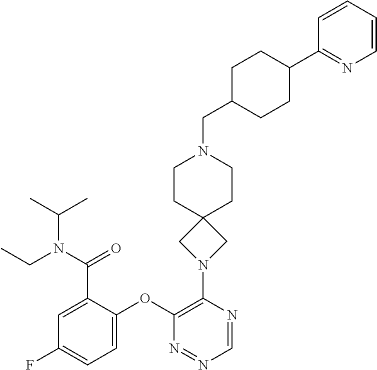 [SMILES: CCN(C(=O)c1cc(F)ccc1Oc1nncnc1N1CC2(CCN(CC3CCC(c4ccccn4)CC3)CC2)C1)C(C)C] |
| 136 | 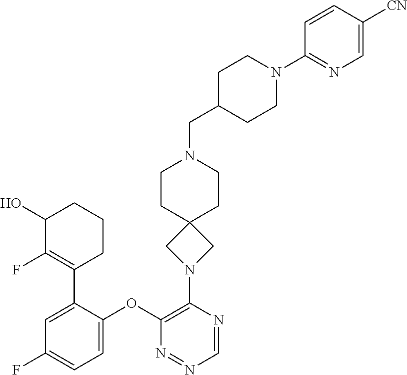 [SMILES: N#Cc1ccc(N2CCC(CN3CCC4(CC3)CN(c3ncnnc3Oc3ccc(F)cc3C3=C(F)C(O)CCC3)C4)CC2)nc1] |
| 137 | 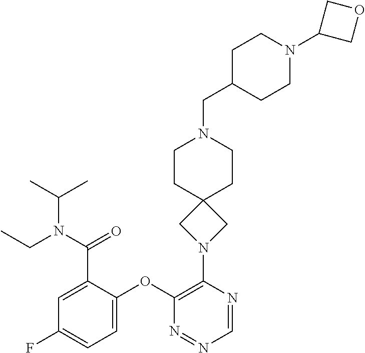 [SMILES: CCN(C(=O)c1cc(F)ccc1Oc1nncnc1N1CC2(CCN(CC3CCN(C4COC4)CC3)CC2)C1)C(C)C] |
| 138 | 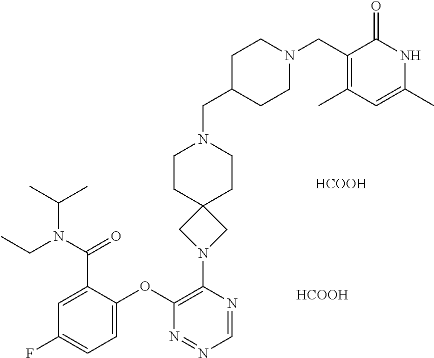 [SMILES: CCN(C(=O)c1cc(F)ccc1Oc1nncnc1N1CC2(CCN(CC3CCN(Cc4c(C)cc(C)[nH]c4=O)CC3)CC2)C1)C(C)C ; "HCOOH" ; "HCOOH"] |
| 139 | 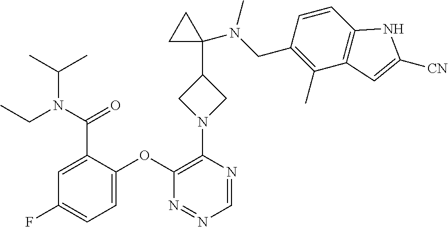 [SMILES: CCN(C(=O)c1cc(F)ccc1Oc1nncnc1N1CC(C2(N(C)Cc3ccc4[nH]c(C#N)cc4c3C)CC2)C1)C(C)C] |
| 140 | 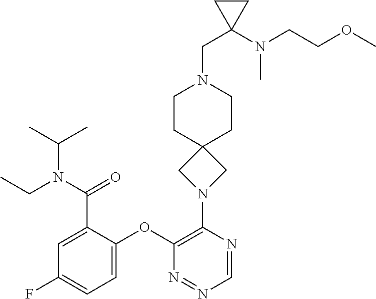 [SMILES: CCN(C(=O)c1cc(F)ccc1Oc1nncnc1N1CC2(CCN(CC3(N(C)CCOC)CC3)CC2)C1)C(C)C] |
| 141 | 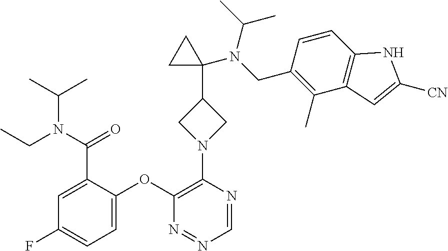 [SMILES: CCN(C(=O)c1cc(F)ccc1Oc1nncnc1N1CC(C2(N(Cc3ccc4[nH]c(C#N)cc4c3C)C(C)C)CC2)C1)C(C)C] |
| 142 | 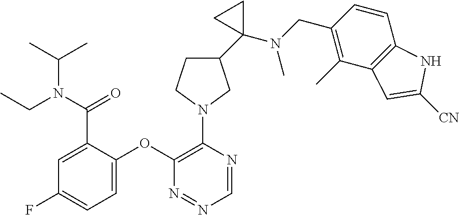 [SMILES: CCN(C(=O)c1cc(F)ccc1Oc1nncnc1N1CCC(C2(N(C)Cc3ccc4[nH]c(C#N)cc4c3C)CC2)C1)C(C)C] |
| 143 | 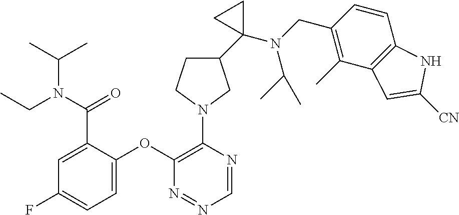 [SMILES: CCN(C(=O)c1cc(F)ccc1Oc1nncnc1N1CCC(C2(N(Cc3ccc4[nH]c(C#N)cc4c3C)C(C)C)CC2)C1)C(C)C] |
| 144 | 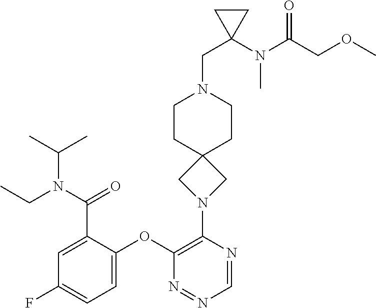 [SMILES: CCN(C(=O)c1cc(F)ccc1Oc1nncnc1N1CC2(CCN(CC3(N(C)C(=O)COC)CC3)CC2)C1)C(C)C] |
| 145 | 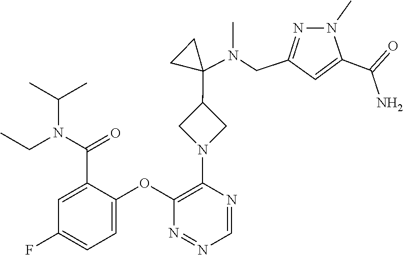 [SMILES: CCN(C(=O)c1cc(F)ccc1Oc1nncnc1N1CC(C2(N(C)Cc3cc(C(N)=O)n(C)n3)CC2)C1)C(C)C] |
| 146 | 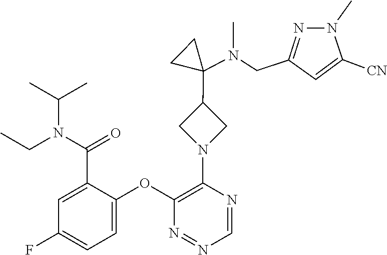 [SMILES: CCN(C(=O)c1cc(F)ccc1Oc1nncnc1N1CC(C2(N(C)Cc3cc(C#N)n(C)n3)CC2)C1)C(C)C] |
| 147 | 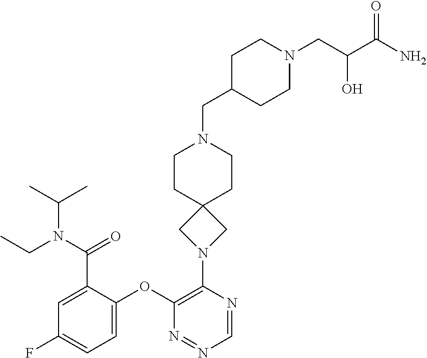 [SMILES: CCN(C(=O)c1cc(F)ccc1Oc1nncnc1N1CC2(CCN(CC3CCN(CC(O)C(N)=O)CC3)CC2)C1)C(C)C] |
| 148 | 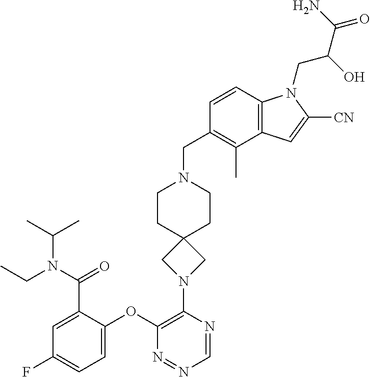 [SMILES: CCN(C(=O)c1cc(F)ccc1Oc1nncnc1N1CC2(CCN(Cc3ccc4c(cc(C#N)n4CC(O)C(N)=O)c3C)CC2)C1)C(C)C] |
| 149 | 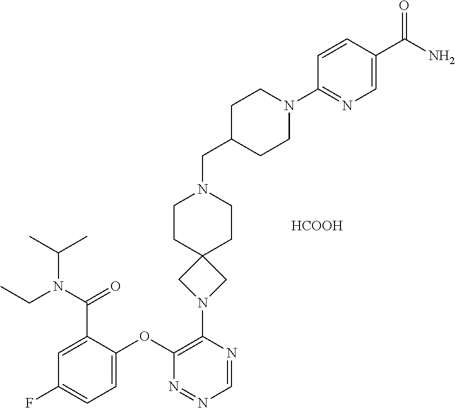 [SMILES: CCN(C(=O)c1cc(F)ccc1Oc1nncnc1N1CC2(CCN(CC3CCN(c4ccc(C(N)=O)cn4)CC3)CC2)C1)C(C)C ; "HCOOH"] |
| 150 | 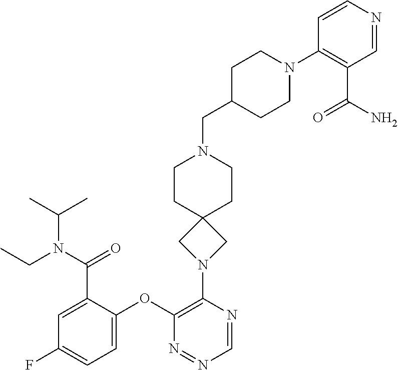 [SMILES: CCN(C(=O)c1cc(F)ccc1Oc1nncnc1N1CC2(CCN(CC3CCN(c4ccncc4C(N)=O)CC3)CC2)C1)C(C)C] |
| 151 | 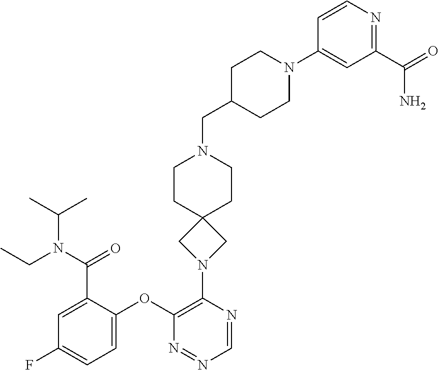 [SMILES: CCN(C(=O)c1cc(F)ccc1Oc1nncnc1N1CC2(CCN(CC3CCN(c4ccnc(C(N)=O)c4)CC3)CC2)C1)C(C)C] |
| 152 | 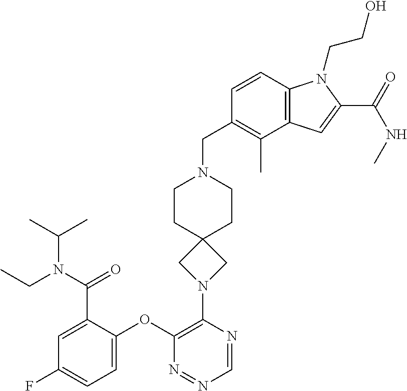 [SMILES: CCN(C(=O)c1cc(F)ccc1Oc1nncnc1N1CC2(CCN(Cc3ccc4c(cc(C(=O)NC)n4CCO)c3C)CC2)C1)C(C)C] |
| 153 | 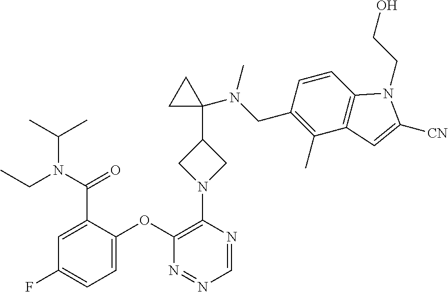 [SMILES: CCN(C(=O)c1cc(F)ccc1Oc1nncnc1N1CC(C2(N(C)Cc3ccc4c(cc(C#N)n4CCO)c3C)CC2)C1)C(C)C] |
| 154 | 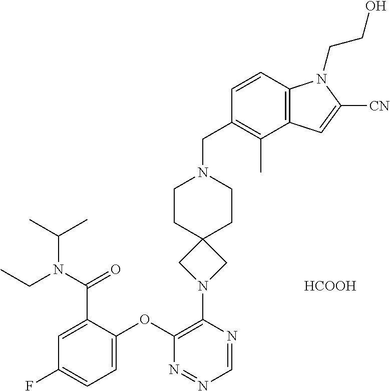 [SMILES: CCN(C(=O)c1cc(F)ccc1Oc1nncnc1N1CC2(CCN(Cc3ccc4c(cc(C#N)n4CCO)c3C)CC2)C1)C(C)C ; "HCOOH"] |
| 155 | 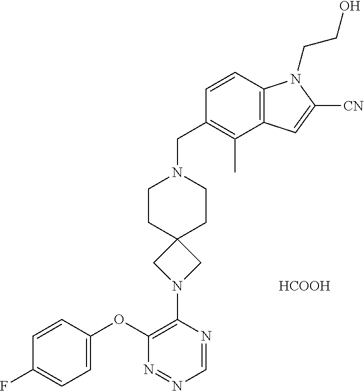 [SMILES: Cc1c(CN2CCC3(CC2)CN(c2ncnnc2Oc2ccc(F)cc2)C3)ccc2c1cc(C#N)n2CCO ; "HCOOH"] |
| 156 | 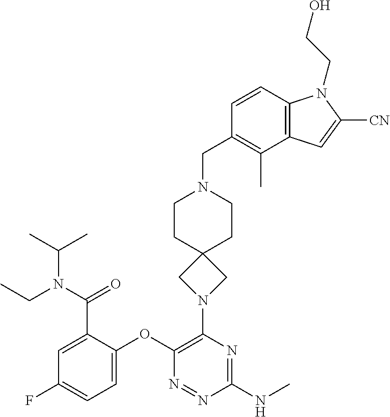 [SMILES: CCN(C(=O)c1cc(F)ccc1Oc1nnc(NC)nc1N1CC2(CCN(Cc3ccc4c(cc(C#N)n4CCO)c3C)CC2)C1)C(C)C] |
| 157 | 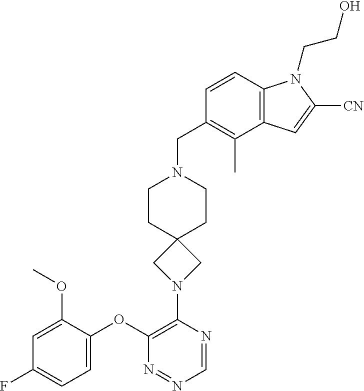 [SMILES: COc1cc(F)ccc1Oc1nncnc1N1CC2(CCN(Cc3ccc4c(cc(C#N)n4CCO)c3C)CC2)C1] |
| 158 | 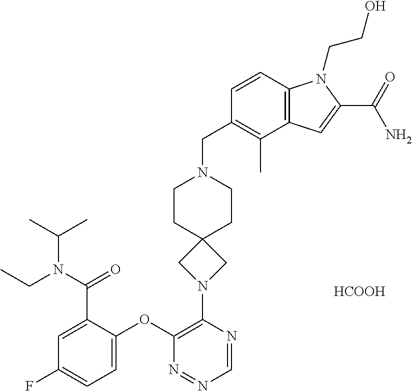 [SMILES: CCN(C(=O)c1cc(F)ccc1Oc1nncnc1N1CC2(CCN(Cc3ccc4c(cc(C(N)=O)n4CCO)c3C)CC2)C1)C(C)C ; "HCOOH"] |
| 159 | 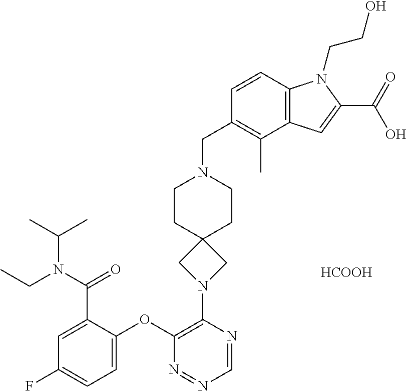 [SMILES: CCN(C(=O)c1cc(F)ccc1Oc1nncnc1N1CC2(CCN(Cc3ccc4c(cc(C(=O)O)n4CCO)c3C)CC2)C1)C(C)C ; "HCOOH"] |
| 160 | 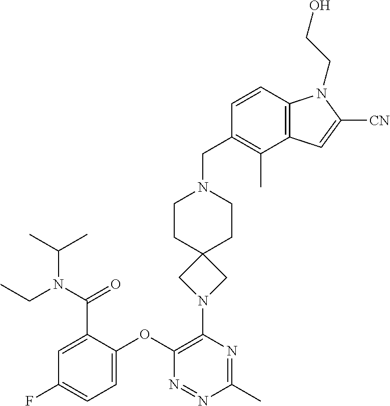 [SMILES: CCN(C(=O)c1cc(F)ccc1Oc1nnc(C)nc1N1CC2(CCN(Cc3ccc4c(cc(C#N)n4CCO)c3C)CC2)C1)C(C)C] |
| 161 | 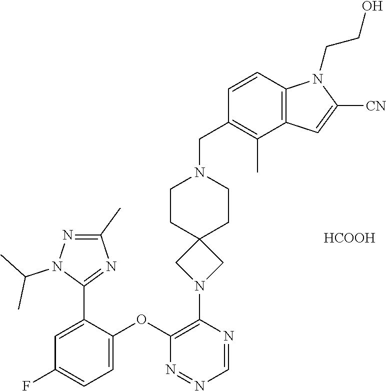 [SMILES: Cc1nc(-c2cc(F)ccc2Oc2nncnc2N2CC3(CCN(Cc4ccc5c(cc(C#N)n5CCO)c4C)CC3)C2)n(C(C)C)n1 ; "HCOOH"] |
| 162 | 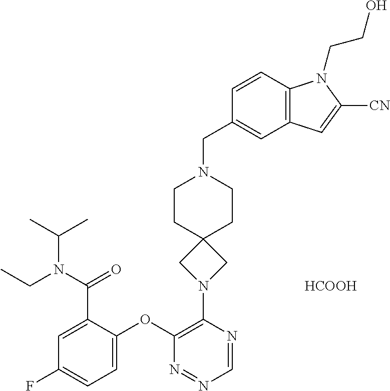 [SMILES: CCN(C(=O)c1cc(F)ccc1Oc1nncnc1N1CC2(CCN(Cc3ccc4c(c3)cc(C#N)n4CCO)CC2)C1)C(C)C ; "HCOOH"] |
| 163 | 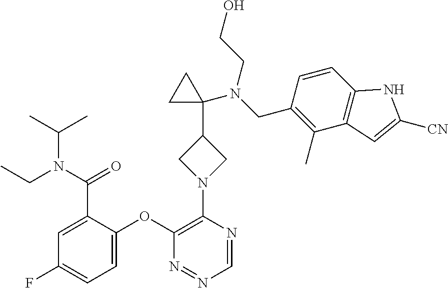 [SMILES: CCN(C(=O)c1cc(F)ccc1Oc1nncnc1N1CC(C2(N(CCO)Cc3ccc4[nH]c(C#N)cc4c3C)CC2)C1)C(C)C] |
| 164 | 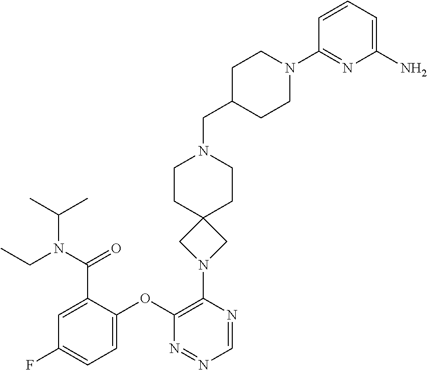 [SMILES: CCN(C(=O)c1cc(F)ccc1Oc1nncnc1N1CC2(CCN(CC3CCN(c4cccc(N)n4)CC3)CC2)C1)C(C)C] |
| 165 | 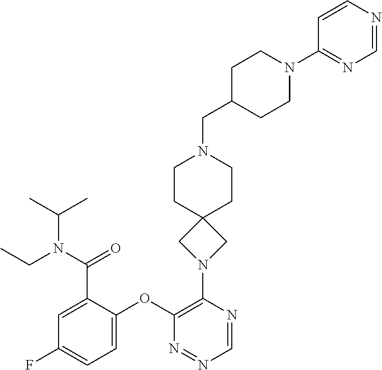 [SMILES: CCN(C(=O)c1cc(F)ccc1Oc1nncnc1N1CC2(CCN(CC3CCN(c4ccncn4)CC3)CC2)C1)C(C)C] |
| 166 | 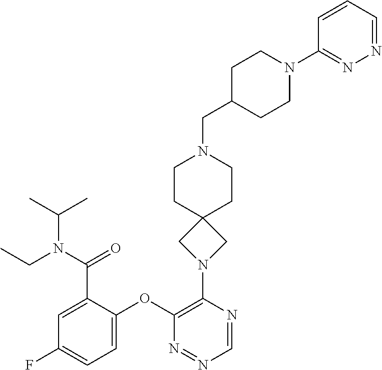 [SMILES: CCN(C(=O)c1cc(F)ccc1Oc1nncnc1N1CC2(CCN(CC3CCN(c4cccnn4)CC3)CC2)C1)C(C)C] |
| 167 | 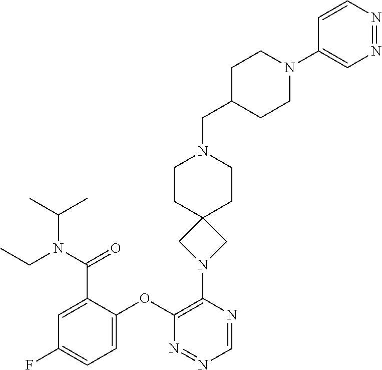 [SMILES: CCN(C(=O)c1cc(F)ccc1Oc1nncnc1N1CC2(CCN(CC3CCN(c4ccnnc4)CC3)CC2)C1)C(C)C] |
| 168 | 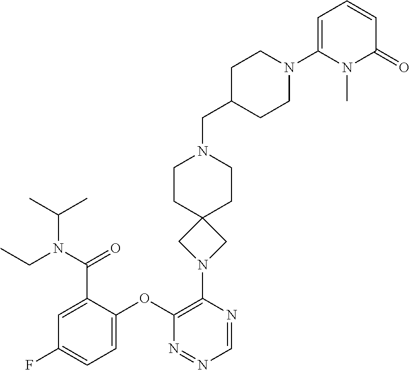 [SMILES: CCN(C(=O)c1cc(F)ccc1Oc1nncnc1N1CC2(CCN(CC3CCN(c4cccc(=O)n4C)CC3)CC2)C1)C(C)C] |
| 169 | 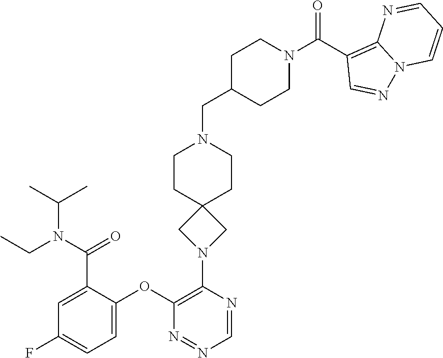 [SMILES: CCN(C(=O)c1cc(F)ccc1Oc1nncnc1N1CC2(CCN(CC3CCN(C(=O)c4cnn5cccnc45)CC3)CC2)C1)C(C)C] |
| 170 | 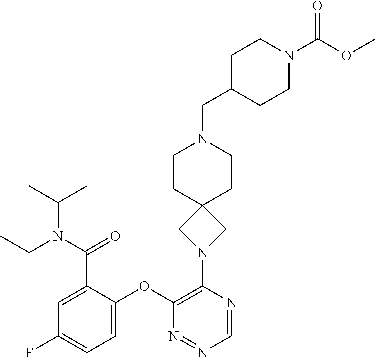 [SMILES: CCN(C(=O)c1cc(F)ccc1Oc1nncnc1N1CC2(CCN(CC3CCN(C(=O)OC)CC3)CC2)C1)C(C)C] |
| 171 | 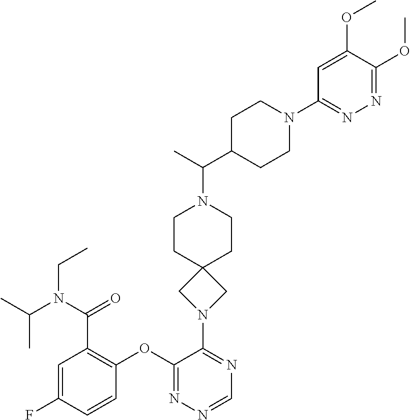 [SMILES: CCN(C(=O)c1cc(F)ccc1Oc1nncnc1N1CC2(CCN(C(C)C3CCN(c4cc(OC)c(OC)nn4)CC3)CC2)C1)C(C)C] |
| 172 | 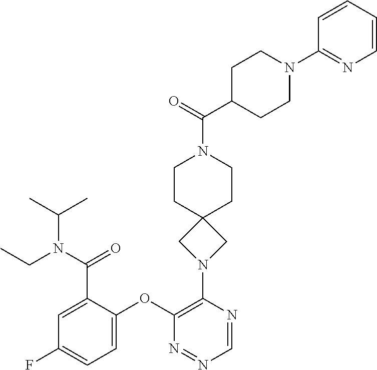 [SMILES: CCN(C(=O)c1cc(F)ccc1Oc1nncnc1N1CC2(CCN(C(=O)C3CCN(c4ccccn4)CC3)CC2)C1)C(C)C] |
| 173 | 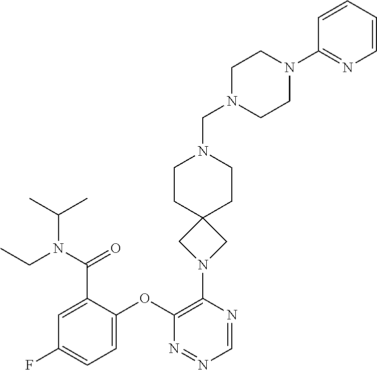 [SMILES: CCN(C(=O)c1cc(F)ccc1Oc1nncnc1N1CC2(CCN(CN3CCN(c4ccccn4)CC3)CC2)C1)C(C)C] |
| 174 | 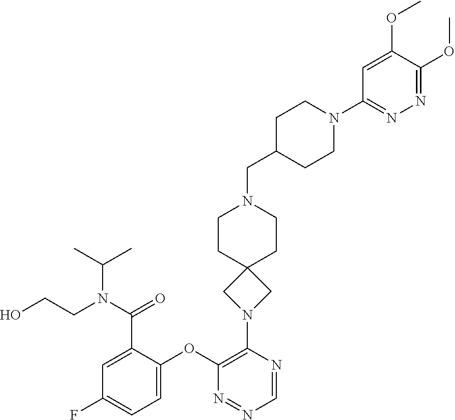 [SMILES: COc1cc(N2CCC(CN3CCC4(CC3)CN(c3ncnnc3Oc3ccc(F)cc3C(=O)N(CCO)C(C)C)C4)CC2)nnc1OC] |
| 175 | 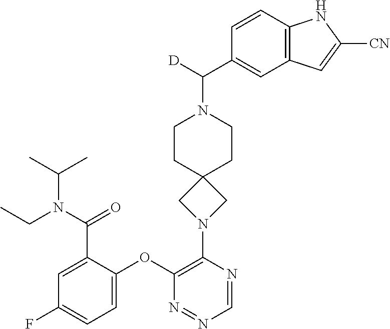 [SMILES: [2H]C(c1ccc2[nH]c(C#N)cc2c1)N1CCC2(CC1)CN(c1ncnnc1Oc1ccc(F)cc1C(=O)N(CC)C(C)C)C2] |
| 176 | 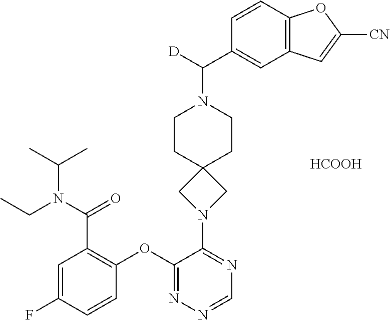 [SMILES: [2H]C(c1ccc2oc(C#N)cc2c1)N1CCC2(CC1)CN(c1ncnnc1Oc1ccc(F)cc1C(=O)N(CC)C(C)C)C2 ; "HCOOH"] |
| 177 | 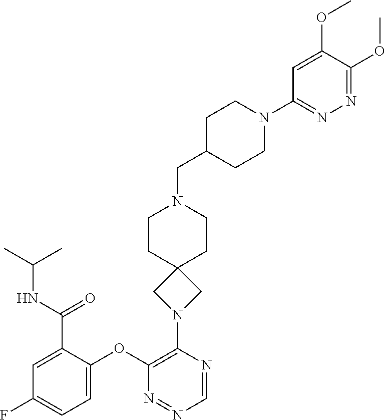 [SMILES: COc1cc(N2CCC(CN3CCC4(CC3)CN(c3ncnnc3Oc3ccc(F)cc3C(=O)NC(C)C)C4)CC2)nnc1OC] |
| 178 | 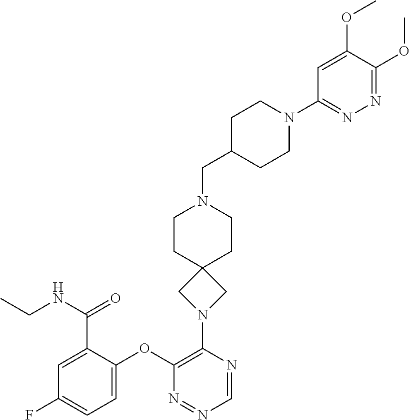 [SMILES: CCNC(=O)c1cc(F)ccc1Oc1nncnc1N1CC2(CCN(CC3CCN(c4cc(OC)c(OC)nn4)CC3)CC2)C1] |
| 179 | 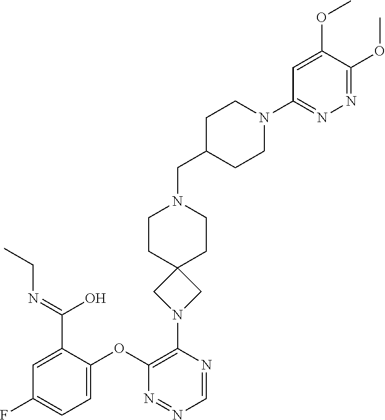 [SMILES: CC/N=C(\O)c1cc(F)ccc1Oc1nncnc1N1CC2(CCN(CC3CCN(c4cc(OC)c(OC)nn4)CC3)CC2)C1] |
| 180 | 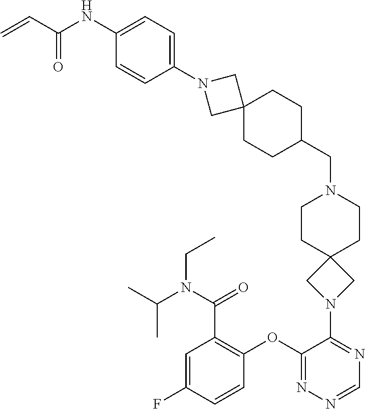 [SMILES: C=CC(=O)Nc1ccc(N2CC3(CCC(CN4CCC5(CC4)CN(c4ncnnc4Oc4ccc(F)cc4C(=O)N(CC)C(C)C)C5)CC3)C2)cc1] |
| 181 | 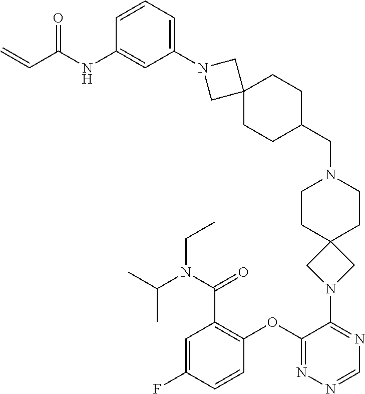 [SMILES: C=CC(=O)Nc1cccc(N2CC3(CCC(CN4CCC5(CC4)CN(c4ncnnc4Oc4ccc(F)cc4C(=O)N(CC)C(C)C)C5)CC3)C2)c1] |
| 182 | 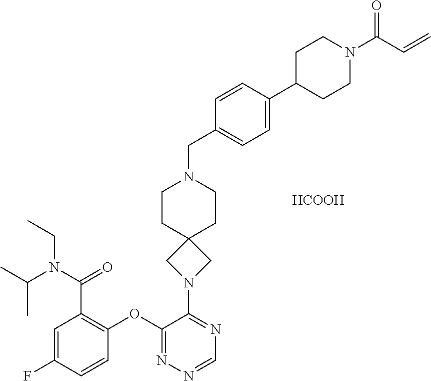 [SMILES: C=CC(=O)N1CCC(c2ccc(CN3CCC4(CC3)CN(c3ncnnc3Oc3ccc(F)cc3C(=O)N(CC)C(C)C)C4)cc2)CC1 ; "HCOOH"] |
| 183 | 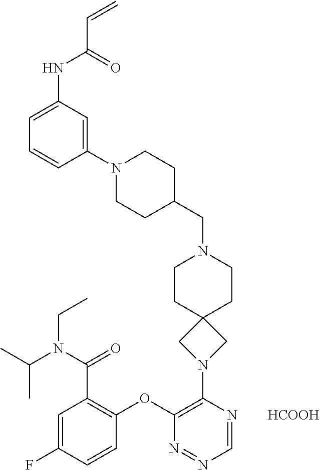 [SMILES: C=CC(=O)Nc1cccc(N2CCC(CN3CCC4(CC3)CN(c3ncnnc3Oc3ccc(F)cc3C(=O)N(CC)C(C)C)C4)CC2)c1 ; "HCOOH"] |
| 184 | 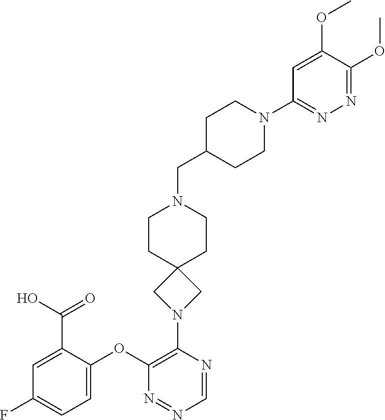 [SMILES: COc1cc(N2CCC(CN3CCC4(CC3)CN(c3ncnnc3Oc3ccc(F)cc3C(=O)O)C4)CC2)nnc1OC] |

Embodiment 19. A pharmaceutical composition, comprising the compound and/or the pharmaceutically acceptable salt thereof according to any one of embodiments 1-18, and optionally comprising a pharmaceutically acceptable excipient.

Embodiment 20. A method of in vivo or in vitro inhibiting menin-MLL interaction, comprising contacting menin with an effective amount of the compound and/or the pharmaceutically acceptable salt thereof according to any one of embodiments 1-18.

Embodiment 21. Use of the compound and/or the pharmaceutically acceptable salt thereof according to any one of embodiments 1-18 in the manufacture of a medicament for treating or preventing a disease mediated by menin-MLL interaction or at least in part by menin-MLL interaction, wherein the disease mediated by menin-MLL interaction or at least in part by menin-MLL interaction is preferably cancer; the cancer is preferably a hematologic malignancy or solid tumor, including leukemia, lymphoma and myeloma; and the cancer is more preferably selected from acute leukemia, chronic leukemia, myeloid leukemia, myelogenous leukemia, lymphocytic leukemia, lymphoblastic leukemia, acute myelogenous leukemia (AML), chronic myelogenous leukemia (CML), acute lymphocytic leukemia (ALL), B-cell acute lymphocytic leukemia (B-ALL), T-cell prolymphocytic leukemia (T-PLL), chronic lymphocytic leukemia (CLL), chronic myelocytic leukemia, large granular lymphocytic leukemia, hairy cell leukemia (HCL), mixed lineage leukemia (MLL), MLL-related leukemia, MLL-rearranged leukemia (MLL-r), MLL-PTD leukemia, MLL-positive leukemia, NPM1 mutant leukemia, leukemia exhibiting HOX/MEIS1 gene expression signatures, myelodysplastic syndrome (MDS), myeloproliferative neoplasm (MPN), multiple myeloma (MM), Hodgkin's lymphoma, non-Hodgkin's lymphoma, diffuse large B-cell lymphoma (DLBCL), B-cell lymphoma, T-cell lymphoma, mantle cell lymphoma, follicular lymphoma (FL), Waldenstrom's macroglobulinemia, prostate cancer, breast cancer, lung cancer, liver cancer, colon cancer, colorectal cancer, pancreatic cancer, melanoma, and glioblastoma (GBM).

Embodiment 22. A method of treating or preventing a disease in a subject, comprising administering to the subject in need thereof an effective amount of the compound and/or the pharmaceutically acceptable salt thereof according to any one of embodiments 1-18, wherein the disease is a disease mediated by menin-MLL interaction or at least in part by menin-MLL interaction; the disease is preferably cancer; the cancer is preferably a hematologic malignancy or solid tumor, including leukemia, lymphoma and myeloma; and the cancer is more preferably selected from acute leukemia, chronic leukemia, myeloid leukemia, myelogenous leukemia, lymphocytic leukemia, lymphoblastic leukemia, acute myelogenous leukemia (AML), chronic myelogenous leukemia (CML), acute lymphocytic leukemia (ALL), B-cell acute lymphocytic leukemia (B-ALL), T-cell prolymphocytic leukemia (T-PLL), chronic lymphocytic leukemia (CLL), chronic myelocytic leukemia, large granular lymphocytic leukemia, hairy cell leukemia (HCL), mixed lineage leukemia (MLL), MLL-related leukemia, MLL-rearranged leukemia (MLL-r), MLL-PTD leukemia, MLL-positive leukemia, NPM1 mutant leukemia, leukemia exhibiting HOX/MEIS1 gene expression signatures, myelodysplastic syndrome (MDS), myeloproliferative neoplasm (MPN), multiple myeloma (MM), Hodgkin's lymphoma, non-Hodgkin's lymphoma, diffuse large B-cell lymphoma (DLBCL), B-cell lymphoma, T-cell lymphoma, mantle cell lymphoma, follicular lymphoma (FL), Waldenstrom's macroglobulinemia, prostate cancer, breast cancer, lung cancer, liver cancer, colon cancer, colorectal cancer, pancreatic cancer, melanoma, and glioblastoma (GBM).

Embodiment 23. The compound and/or the pharmaceutically acceptable salt thereof according to any one of embodiments 1-18, for use as a medicament.

Embodiment 24. The compound and/or the pharmaceutically acceptable salt thereof according to any one of embodiments 1-18, for use in treating or preventing a disease mediated by menin-MLL interaction or at least in part by menin-MLL interaction, wherein the disease is preferably cancer; the cancer is preferably a hematologic malignancy or solid tumor, including leukemia, lymphoma and myeloma; and the cancer is more preferably selected from acute leukemia, chronic leukemia, myeloid leukemia, myelogenous leukemia, lymphocytic leukemia, lymphoblastic leukemia, acute myelogenous leukemia (AML), chronic myelogenous leukemia (CML), acute lymphocytic leukemia (ALL), B-cell acute lymphocytic leukemia (B-ALL), T-cell prolymphocytic leukemia (T-PLL), chronic lymphocytic leukemia (CLL), chronic myelocytic leukemia, large granular lymphocytic leukemia, hairy cell leukemia (HCL), mixed lineage leukemia (MLL), MLL-related leukemia, MLL-rearranged leukemia (MLL-r), MLL-PTD leukemia, MLL-positive leukemia, NPM1 mutant leukemia, leukemia exhibiting HOX/MEIS1 gene expression signatures, myelodysplastic syndrome (MDS), myeloproliferative neoplasm (MPN), multiple myeloma (MM), Hodgkin's lymphoma, non-Hodgkin's lymphoma, diffuse large B-cell lymphoma (DLBCL), B-cell lymphoma, T-cell lymphoma, mantle cell lymphoma, follicular lymphoma (FL), Waldenstrom's macroglobulinemia, prostate cancer, breast cancer, lung cancer, liver cancer, colon cancer, colorectal cancer, pancreatic cancer, melanoma, and glioblastoma (GBM).

Embodiment 25. A pharmaceutical combination, comprising the compound and/or the pharmaceutically acceptable salt thereof according to any one of embodiments 1-18, and at least one additional therapeutic agent, wherein the additional therapeutic agent is preferably selected from an anti-neoplastic active agent, an anti-inflammatory agent or an immunomodulator, wherein the anti-neoplastic active agent includes a chemotherapeutic agent, an immune checkpoint inhibitor or agonist, and a targeted therapeutic agent.

Embodiment 26. A compound of formula (II):

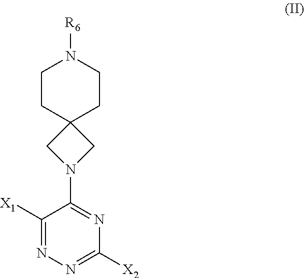

or a pharmaceutically acceptable salt, a solvate, a racemic mixture, an enantiomer, a diastereomer or a tautomer thereof, or a deuterate thereof, wherein
R₆ is an amino protecting group; preferably, R₆ is an amino protecting group selected from Boc (tert-butoxycarbonyl), benzyl, Pmb (p-methoxybenzyl) and Cbz (benzyloxycarbonyl); and more preferably, R₆ is Boc (tert-butoxycarbonyl);
X₁ is halogen; preferably, X, is chlorine;
X₂ is hydrogen or halogen; preferably, X₂ is hydrogen or chlorine; and more preferably, X₂ is chlorine.

Embodiment 27. The compound according to embodiment 26, which is

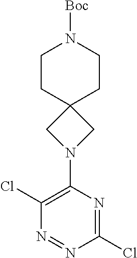 [SMILES: CC(C)(C)OC(=O)N1CCC2(CC1)CN(c1nc(Cl)nnc1Cl)C2]

or a pharmaceutically acceptable salt, a solvate, a racemic mixture, an enantiomer, a diastereomer or a tautomer thereof, or a deuterate thereof.

Embodiment 28. A compound of formula (III):

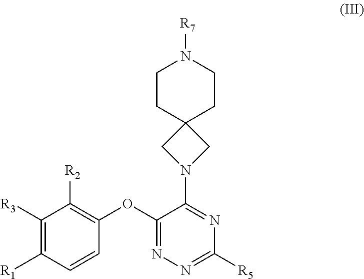

or a pharmaceutically acceptable salt, a solvate, a racemic mixture, an enantiomer, a diastereomer or a tautomer thereof, or a deuterate thereof, wherein
R₁, R₂, R₃ and R₅ are as defined in any one of embodiments 1-17;
R₇ is hydrogen or an amino protecting group; preferably, R₇ is hydrogen or an amino protecting group selected from Boc (tert-butoxycarbonyl), benzyl, Pmb (p-methoxybenzyl) and Cbz (benzyloxycarbonyl); and more preferably, R₇ is hydrogen or Boc (tert-butoxycarbonyl).

Embodiment 28.1. The compound according to embodiment 28, wherein, when R₅ is H or Cl, R₂ is not —C(O)N(CH(CH₃)₂)₂, —C(O)N(CH(CH₃)₂)(CH₃), —C(O)N(CH(CH₃)₂)(CH₂CH₃), —C(O)N(CH(CH₃)₂)(CH₂CHF₂), —C(O)N(CH(CH₃)₂)(CH₂CF₃), —C(O)N(CH(CH₃)₂)(CH₂CH₂OH), —C(O)N(cyclopropyl)₂, —C(O)N(CH(CH₃)₂)(cyclopropyl), —C(O)N(CH₂CH₃)(cyclopropyl), —C(O)N(CH(CH₃)₂)(cyclobutyl), pyrimidyl substituted with isopropyl, and pyrazolyl substituted with isopropyl and/or Cl.

Embodiment 29. The compound according to embodiment 28, which is selected from:

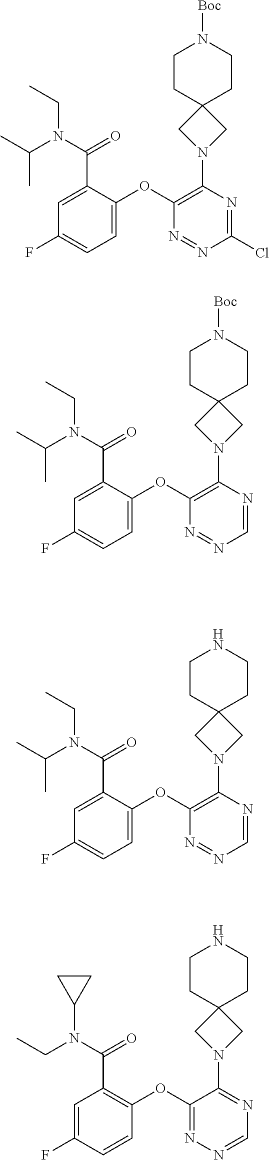 [SMILES: CCN(C(=O)c1cc(F)ccc1Oc1nnc(Cl)nc1N1CC2(CCN(C(=O)OC(C)(C)C)CC2)C1)C(C)C ; CCN(C(=O)c1cc(F)ccc1Oc1nncnc1N1CC2(CCN(C(=O)OC(C)(C)C)CC2)C1)C(C)C ; CCN(C(=O)c1cc(F)ccc1Oc1nncnc1N1CC2(CCNCC2)C1)C(C)C ; CCN(C(=O)c1cc(F)ccc1Oc1nncnc1N1CC2(CCNCC2)C1)C1CC1]

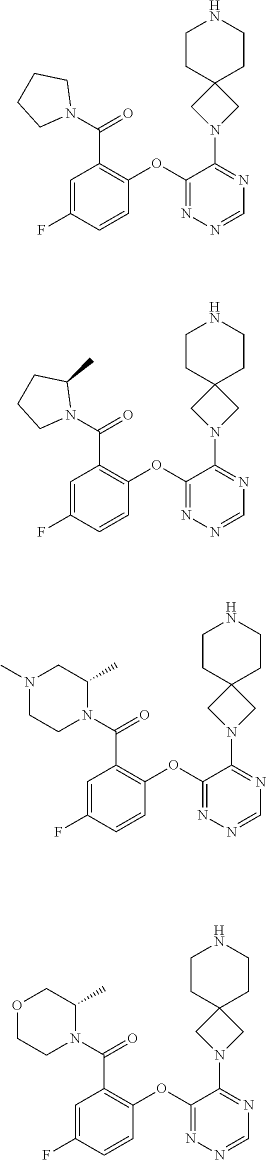 [SMILES: O=C(c1cc(F)ccc1Oc1nncnc1N1CC2(CCNCC2)C1)N1CCCC1 ; C[C@@H]1CCCN1C(=O)c1cc(F)ccc1Oc1nncnc1N1CC2(CCNCC2)C1 ; C[C@H]1CN(C)CCN1C(=O)c1cc(F)ccc1Oc1nncnc1N1CC2(CCNCC2)C1 ; C[C@H]1COCCN1C(=O)c1cc(F)ccc1Oc1nncnc1N1CC2(CCNCC2)C1]

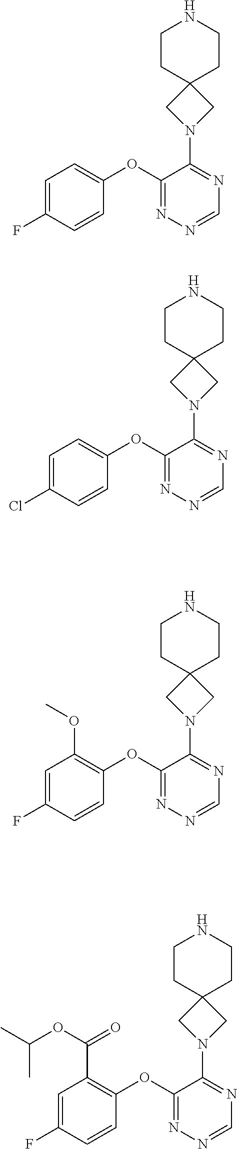 [SMILES: Fc1ccc(Oc2nncnc2N2CC3(CCNCC3)C2)cc1 ; Clc1ccc(Oc2nncnc2N2CC3(CCNCC3)C2)cc1 ; COc1cc(F)ccc1Oc1nncnc1N1CC2(CCNCC2)C1 ; CC(C)OC(=O)c1cc(F)ccc1Oc1nncnc1N1CC2(CCNCC2)C1]

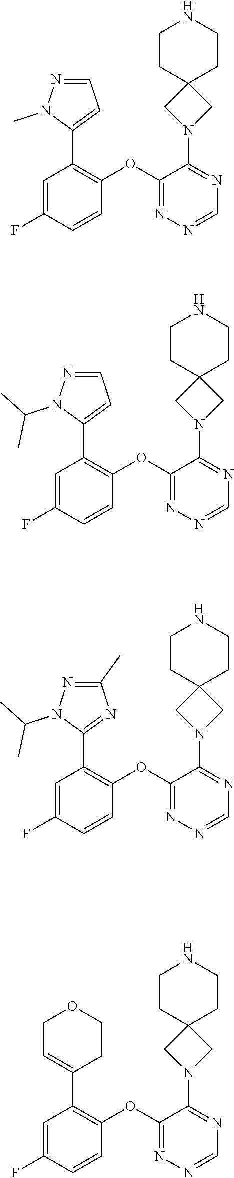 [SMILES: Cn1nccc1-c1cc(F)ccc1Oc1nncnc1N1CC2(CCNCC2)C1 ; CC(C)n1nccc1-c1cc(F)ccc1Oc1nncnc1N1CC2(CCNCC2)C1 ; Cc1nc(-c2cc(F)ccc2Oc2nncnc2N2CC3(CCNCC3)C2)n(C(C)C)n1 ; Fc1ccc(Oc2nncnc2N2CC3(CCNCC3)C2)c(C2=CCOCC2)c1]

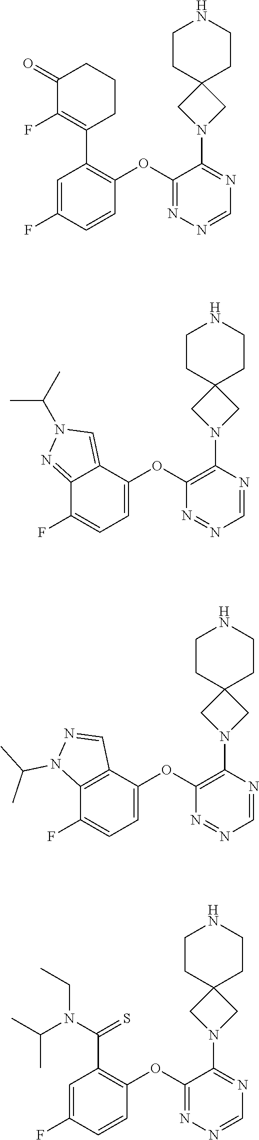 [SMILES: O=C1CCCC(c2cc(F)ccc2Oc2nncnc2N2CC3(CCNCC3)C2)=C1F ; CC(C)n1cc2c(Oc3nncnc3N3CC4(CCNCC4)C3)ccc(F)c2n1 ; CC(C)n1ncc2c(Oc3nncnc3N3CC4(CCNCC4)C3)ccc(F)c21 ; CCN(C(=S)c1cc(F)ccc1Oc1nncnc1N1CC2(CCNCC2)C1)C(C)C]

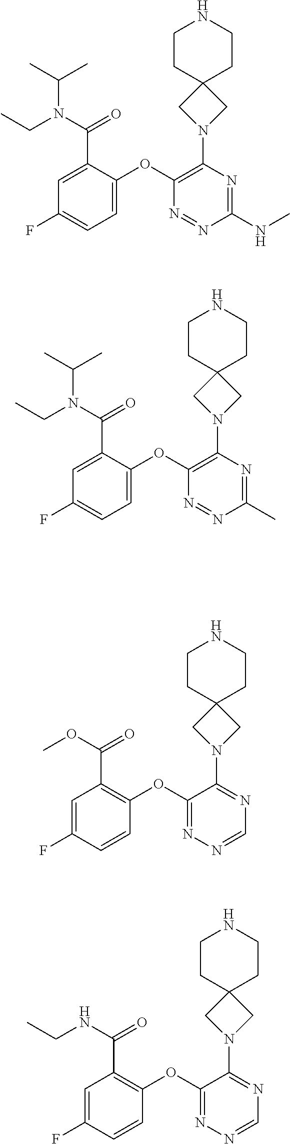

or a pharmaceutically acceptable salt, a solvate, a racemic mixture, an enantiomer, a diastereomer or a tautomer thereof, or a deuterate thereof.

The various embodiments of the present invention (including the following examples) and the features of the various embodiments should be interpreted as being arbitrarily combined with each other, and the various solutions obtained from these mutual combinations are all included in the scope of the present invention, just like the solutions obtained from the mutual combinations specifically and individually set forth herein, unless clearly stated otherwise in the context.

### BENEFICIAL EFFECTS OF THE INVENTION

As described above, it is known that the binding and interaction between MLL fusion protein and menin protein is very important for MLL protein to exert its oncogenic function. Inhibiting or interfering with the MLL-menin interaction can significantly inhibit the cell proliferation in MLL fusion protein-related cancer diseases. We have surprisingly found that the compounds of the present invention having the above-mentioned structural features can significantly inhibit the binding activity of menin and MLL proteins, and can potently inhibit the cell proliferation in cell lines carrying mutations, thereby having potential value as anti-proliferative, pro-apoptotic and/or anti-invasive medicaments for preventing, suppressing and/or treating diseases mediated by menin-MLL interaction or at least in part by menin-MLL interaction, such as cancer or tumor as defined herein.

Specifically, studies have found that the compounds of the present invention can achieve one or more of the following technical effects:
potent MLL-menin binding inhibitory activity: the compounds of the present invention, especially the example compounds herein, show inhibitory activity on the protein binding in MLL and menin protein binding assay (fluorescence polarization method) at a molecular level, with IC50 values generally in the range of 0.1 nM-10 μM, such as 0.1 nM-5 μM, preferably 0.1 nM-1 μM, more preferably 0.1 nM-100 nM, and most preferably 0.1 nM-50 nM, as verified in example 3;
potent cell proliferation inhibitory activity: the compounds of the present invention, especially the example compounds herein, show potent proliferation inhibitory activity in human acute myelogenous leukemia cell MV-4-11 proliferation inhibition assay, with GI50 values generally in the range of 0.1 nM-10 μM, such as 0.1 nM-5 μM, preferably 0.1 nM-1 μM, more preferably 1 nM-100 nM, and most preferably 1 nM-50 nM, as verified in example 4;
expected good pharmacokinetic properties; and
expected satisfactory safety and less toxic and side effects.

### General Synthetic Methods

The compound of formula (I) and/or a pharmaceutically acceptable salt thereof described herein can be synthesized using commercially available starting materials, by methods known in the art, or methods disclosed in the present patent application. The synthetic routes shown in schemes 1-3 illustrate the general synthetic methods of the compounds of the present invention.

### Method 1:

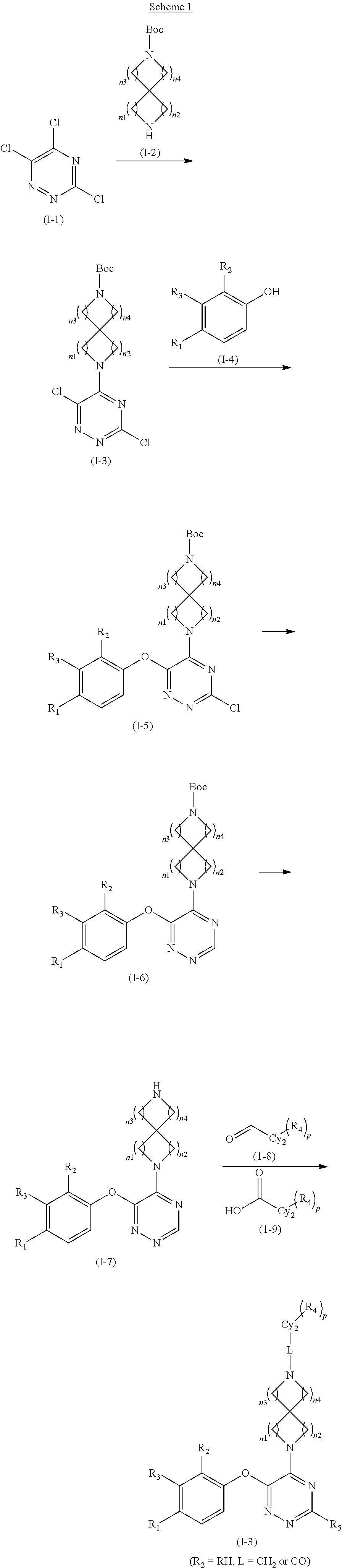

As shown in scheme 1, a compound of formula (1-1) is subjected to a substitution reaction with a compound of formula (1-2) under alkaline conditions (such as but not limited to triethylamine) to obtain a compound of formula (1-3). The compound of formula (1-3) is subjected to a substitution reaction with a compound of formula (1-4) under alkaline conditions (such as but not limited to DBU) to obtain a compound of formula (1-5). The compound of formula (1-5) is reduced and dechlorinated under the action of catalysts (such as but not limited to Pd/C) to produce a compound of formula (1-6), which is subjected to de-Boc reaction under acidic conditions (such as but not limited to HCl) to convert to a compound of formula (1-7). The compound of formula (1-7) is then subjected to a reductive amination reaction with a compound of formula (1-8) under the action of reducing agents (such as but not limited to sodium triacetoxyborohydride, sodium cyanoborohydride, etc.) to obtain a compound of formula (I-3) (wherein R₅═H, and L=CH₂). Alternatively, the compound of formula (1-7) is subjected to a condensation reaction with a compound of formula (1-9) under the action of condensing agents (such as but not limited to HATU) to obtain a compound of formula (I-3) (wherein R₅=H, and L=CO). R₁, R₂, R₃, R₄, Cy₂, n1, n2, n3, n4 and p are as defined herein.

### Method 2:

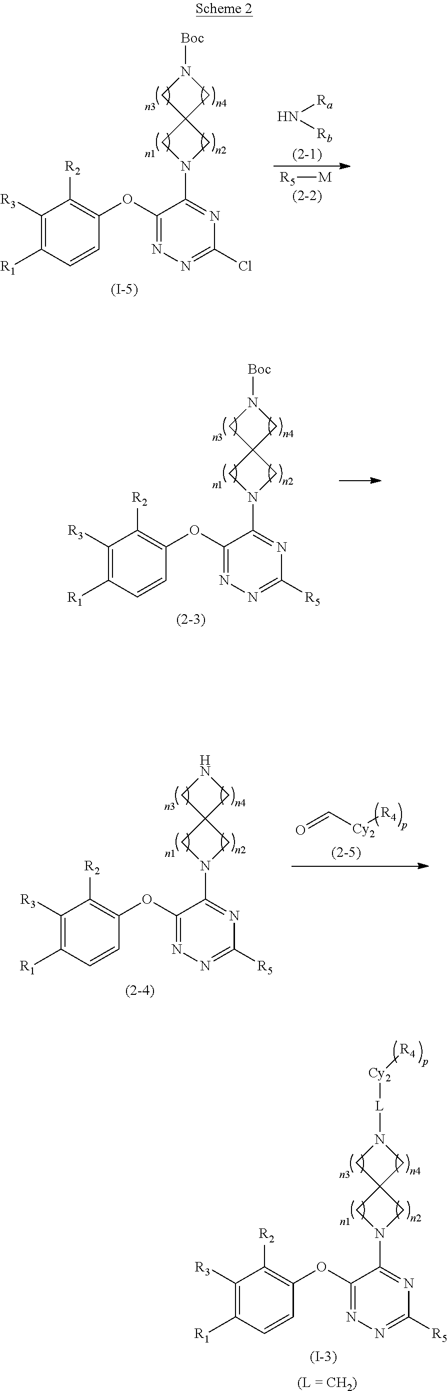

As shown in scheme 2, a compound of formula (1-5) is subjected to a substitution reaction with a compound of formula (2-1) under alkaline conditions (such as but not limited to triethylamine) to produce a compound of formula (2-3) (wherein R₅=—NRₐR_{b}). Alternatively, a compound of formula (1-5) is subjected to a coupling reaction with a compound of formula (2-2) under the catalysis of palladium reagents (such as but not limited to Pd(PPh₃)₄) to obtain a compound of formula (2-3) (wherein R₅=C₁₋₆ alkyl). The compound of formula (2-3) (wherein R₅=—NRₐR_{b} or R₅=C₁₋₆ alkyl) is subjected to de-Boc reaction under acidic conditions (such as but not limited to HCl) to obtain a compound of formula (2-4). The compound of formula (2-4) is then subjected to a reductive amination reaction with a compound of formula (2-5) under the action of reducing agents (such as but not limited to sodium triacetoxyborohydride, sodium cyanoborohydride, etc.) to obtain a compound of formula (1-3) (wherein L=CH₂, and R₅=—NRₐR_{b} or R₅═C₁₋₆ alkyl). R₁, R₂, R₃, R₄, Rₐ, R_{b}, Cy₂, n1, n2, n3, n4 and p are as defined herein; and M is boronic anhydride, borate or boronic acid.

### Method 3:

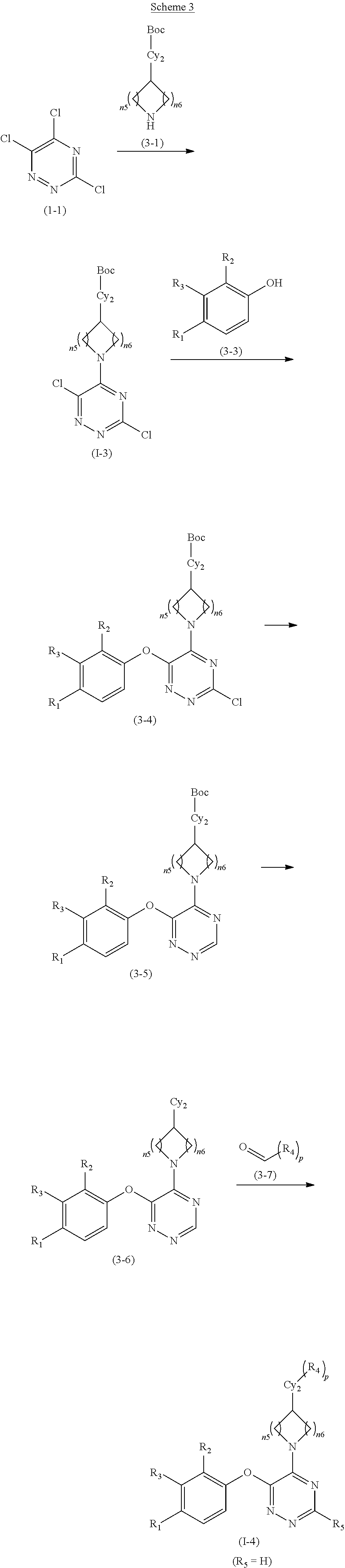

As shown in scheme 3, a compound of formula (1-1) is subjected to a substitution reaction with a compound of formula (3-1) under alkaline conditions (such as but not limited to triethylamine) to obtain a compound of formula (3-2). The compound of formula (3-2) is subjected to a substitution reaction with a compound of formula (3-3) under alkaline conditions (such as but not limited to DBU) to obtain a compound of formula (3-4). The compound of formula (3-4) is reduced and dechlorinated under the action of catalysts (such as but not limited to Pd/C) to produce a compound of formula (3-5). The compound of formula (3-5) is subjected to de-Boc reaction under acidic conditions (such as but not limited to HCl) to obtain a compound of formula (3-6). The compound of formula (3-6) is then subjected to a reductive amination reaction with a compound of formula (3-7) under the action of reducing agents (such as but not limited to sodium triacetoxyborohydride, sodium cyanoborohydride, etc.) to obtain a compound of formula (I-4) (wherein R₅=H). R₁, R₂, R₃, R₄, Cy₂, n5, n6 and p are as defined herein.

The substituents of the compounds thus obtained can be further modified to provide other desired compounds. Synthetic chemistry transformations are described, for example, in R. Larock, Comprehensive Organic Transformations, VCH Publishers (1989); L. Fieser and M. Fieser, Fieser and Fieser's Reagents for Organic Synthesis, John Wiley and Sons (1994); L. Paquette, ed., Encyclopedia of Reagents for Organic Synthesis, John Wiley and Sons (1995) and subsequent editions thereof.

Before use, the compound(s) of the present invention can be purified by column chromatography, high performance liquid chromatography, crystallization or other suitable methods.

### Pharmaceutical Compositions and Utility

The compound of the present invention (e.g., a compound of any of the examples as described herein) is used, alone or in combination with one or more additional therapeutic agents, to formulate pharmaceutical compositions. A pharmaceutical composition comprises: (a) an effective amount of the compounds of the present invention; (b) a pharmaceutically acceptable excipient (e.g., one or more pharmaceutically acceptable carriers); and optionally (c) at least one additional therapeutic agent.

A pharmaceutically acceptable excipient refers to an excipient that is compatible with active ingredients of the composition (and in some embodiments, capable of stabilizing the active ingredients) and not deleterious to the subject to be treated. For example, solubilizing agents, such as cyclodextrins (which form specific, more soluble complexes with the compounds of the present invention), can be utilized as pharmaceutical excipients for delivery of the active ingredients. Examples of other excipients include colloidal silicon dioxide, magnesium stearate, cellulose, sodium lauryl sulfate, and pigments such as D&C Yellow #10. Suitable pharmaceutically acceptable excipients are disclosed in Remington's Pharmaceutical Sciences, A. Osol, a standard reference text in the art.

A pharmaceutical composition comprising a compound of the present invention can be administered in various known manners, such as orally, topically, rectally, parenterally, by inhalation spray, or via an implanted reservoir. The term “parenteral” as used herein includes subcutaneous, intracutaneous, intravenous, intramuscular, intraarticular, intraarterial, intrasynovial, intrasternal, intrathecal, intralesional and intracranial injection or infusion techniques.

A pharmaceutical composition described herein can be prepared in the form of tablet, capsule, sachet, dragee, powder, granule, lozenge, powder for reconstitution, liquid preparation, or suppository. In some embodiments, a pharmaceutical composition comprising a compound of the present invention is formulated for intravenous infusion, topical administration, or oral administration.

An oral composition can be any orally acceptable dosage form including, but not limited to, tablets, capsules, emulsions, and aqueous suspensions, dispersions and solutions. Commonly used carriers for tablets include lactose and corn starch. Lubricating agents, such as magnesium stearate, are also typically added to tablets. For oral administration in a capsule form, useful diluents include lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added.

In some embodiments, the compound of the present invention can be present in an amount of 1, 5, 10, 15, 20, 25, 50, 75, 80, 85, 90, 95, 100, 125, 150, 200, 250, 300, 400 and 500 mg in a tablet. In some embodiments, the compound of the present invention can be present in an amount of 1, 5, 10, 15, 20, 25, 50, 75, 80, 85, 90, 95, 100, 125, 150, 200, 250, 300, 400 and 500 mg in a capsule.

A sterile injectable composition (e.g., aqueous or oleaginous suspension) can be formulated according to techniques known in the art using suitable dispersing or wetting agents (for example, Tween 80) and suspending agents. The sterile injectable composition can also be a sterile injectable solution or suspension in a non-toxic parenterally acceptable diluent or solvent, for example, as a solution in 1,3-butanediol. Among the pharmaceutically acceptable vehicles and solvents that can be employed are mannitol, water, Ringer's solution and isotonic sodium chloride solution. In addition, sterile, fixed oils are conventionally employed as a solvent or suspending medium (e.g., synthetic mono- or di-glycerides). Fatty acids, such as oleic acid and its glyceride derivatives, and natural pharmaceutically acceptable oils, such as olive oil or castor oil, especially in their polyoxyethylated versions, can be used as sterile injectable medium. These oil solutions or suspensions can also contain a long-chain alcohol diluent or dispersant, or carboxymethyl cellulose or similar dispersing agents.

An inhalation composition can be prepared according to techniques well known in the art of pharmaceutical formulation and can be prepared as solutions in saline, employing benzyl alcohol or other suitable preservatives, absorption promoters to enhance bioavailability, fluorocarbons, and/or other solubilizing or dispersing agents known in the art.

A topical composition can be formulated in form of oil, cream, lotion, ointment, and the like. Suitable carriers for the composition include vegetable or mineral oils, white petrolatum (white soft paraffin), branched chain fats or oils, animal fats and high molecular weight alcohols (greater than C12). In some embodiments, the pharmaceutically acceptable carrier is one in which the active ingredient is soluble. Emulsifiers, stabilizers, humectants and antioxidants may also be included as well as agents imparting color or fragrance, if desired. Additionally, transdermal penetration enhancers may be employed in those topical formulations. Examples of such enhancers can be found in U.S. Pat. Nos. 3,989,816 and 4,444,762.

Creams may be formulated from a mixture of mineral oil, self-emulsifying beeswax and water in which mixture the active ingredient, dissolved in a small amount of an oil, such as almond oil, is admixed. An example of such a cream is one which includes, by weight, about 40 parts water, about 20 parts beeswax, about 40 parts mineral oil and about 1 part almond oil. Ointments may be formulated by mixing a solution of the active ingredient in a vegetable oil, such as almond oil, with warm soft paraffin and allowing the mixture to cool. An example of such an ointment is one which includes about 30% by weight almond oil and about 70% by weight white soft paraffin.

Suitable in vitro assays can be used to evaluate the effect of the compounds of the present invention in inhibiting menin-MLL interaction. The compounds of the present invention can further be examined for effects in preventing or treating cancer by in vivo assays. For example, the compound of the present invention can be administered to an animal (e.g., a mouse model) having cancer and its therapeutic effects can be accessed. If the pre-clinical results are successful, the dosage range and administration route for animals, such as humans, can be projected.

The compound of the present invention can be shown to have sufficient pre-clinical practical utility to merit clinical trials hoped to demonstrate a beneficial therapeutic or prophylactic effect, for example, in subjects with cancer.

As used herein, the term “cancer” refers to a cellular disorder characterized by uncontrolled or disregulated cell proliferation, decreased cellular differentiation, inappropriate ability to invade surrounding tissue, and/or ability to establish new growth at ectopic sites. The term “cancer” includes, but is not limited to, solid tumors and hematologic malignancies, such as leukemia, lymphoma or myeloma. The term “cancer” encompasses diseases of skin, tissues, organs, bone, cartilage, blood, and vessels. The term “cancer” further encompasses primary cancer, and metastatic cancer, recurrent cancer and refractory cancer.

Non-limiting examples of solid tumors include pancreatic cancer; bladder cancer; colorectal cancer; colon cancer; breast cancer, including metastatic breast cancer; prostate cancer, including androgen-dependent and androgen-independent prostate cancer; testicular cancer; renal cancer, including, e.g., metastatic renal cell carcinoma; urothelial carcinoma; liver cancer; hepatocellular cancer; lung cancer, including, e.g., non-small cell lung cancer (NSCLC), small cell lung cancer, bronchioloalveolar carcinoma (BAC), and adenocarcinoma of the lung; ovarian cancer, including, e.g., progressive epithelial or primary peritoneal cancer; cervical cancer; endometrial cancer; gastric cancer; esophageal cancer; cholangiocarcinoma; head and neck cancer, including, e.g., squamous cell carcinoma of the head and neck; skin cancer, including, e.g., melanoma and basal carcinoma; neuroendocrine cancer, including metastatic neuroendocrine tumors; brain tumors, including, e.g., glioma, glioblastoma (GBM), anaplastic oligodendroglioma, adult glioblastoma multiforme, and adult anaplastic astrocytoma; bone cancer; sarcoma, including, e.g., Kaposi's sarcoma; adrenal carcinoma; mesothelioma; mesothelial carcinoma; choriocarcinoma; muscle carcinoma; connective tissue carcinoma; and thyroid carcinoma.

Non-limiting examples of hematologic malignancies include acute leukemia; chronic leukemia; myeloid leukemia; myelogenous leukemia; lymphocytic leukemia; chronic lymphoid leukemia; lymphoblastic leukemia; acute lymphoblastic leukemia; acute myelogenous leukemia (AML); juvenile acute myelogenous leukemia; chronic myelogenous leukemia (CML), including accelerated phase CML and CML blastic phase (CML-BP); acute lymphocytic leukemia (ALL); T-cell acute lymphocytic leukemia; B-cell acute lymphocytic leukemia (B-ALL); T-cell prolymphocytic leukemia (T-PLL); chronic lymphocytic leukemia (CLL), including high risk CLL; chronic myelocytic leukemia; large granular lymphocytic leukemia; human acute monocytic leukemia (M(5)); hairy cell leukemia (HCL); mixed lineage leukemia (MLL); MLL-related leukemia; MLL-rearranged leukemia (MLL-r); MLL-PTD leukemia; MLL-positive leukemia; NPM1 mutant leukemia; leukemia exhibiting HOX/MEIS1 gene expression signatures; small lymphotic lymphoma (SLL); lymphoblastic lymphoma; Hodgkin's lymphoma; non-Hodgkin's lymphoma (NHL); mantle cell lymphoma (MCL); B-cell lymphoma; T-cell lymphoma; diffuse large B-cell lymphoma (DLBCL); large B-cell lymphoma (LBCL); follicular lymphoma (FL); marginal zone lymphoma; Burkitt's lymphoma; non-Burkitt's highly degree B cell malignant lymphoma; extranodal marginal-zone B-cell lymphoma; multiple myeloma (MM); Waldenstrom's macroglobulinemia; myeloproliferative neoplasm (MPN); myelodysplastic syndrome (MDS), including refractory anemia (RA), refractory anemia with ring sideroblasts (RARS), refractory anemia with excess of blasts (RAEB) and refractory anemia with excess blasts in transformation (RAEB-T); and myeloproliferative syndrome.

In some embodiments, solid tumor is prostate cancer, breast cancer, lung cancer, liver cancer, colon cancer, colorectal cancer, pancreatic cancer, melanoma, glioblastoma (GBM).

In some embodiments, hematologic malignancy is acute leukemia, chronic leukemia, myeloid leukemia, myelogenous leukemia, lymphocytic leukemia, lymphoblastic leukemia, acute myelogenous leukemia (AML), chronic myelogenous leukemia (CML), acute lymphocytic leukemia (ALL), B-cell acute lymphocytic leukemia (B-ALL), T-cell prolymphocytic leukemia (T-PLL), chronic lymphocytic leukemia (CLL), chronic myelocytic leukemia, large granular lymphocytic leukemia, hairy cell leukemia (HCL), mixed lineage leukemia (MLL), MLL-related leukemia, MLL-rearranged leukemia (MLL-r), MLL-PTD leukemia, MLL-positive leukemia, NPM1 mutant leukemia, leukemia exhibiting HOX/MEIS1 gene expression signatures, myelodysplastic syndrome (MDS), myeloproliferative neoplasm (MPN), multiple myeloma (MM), Hodgkin's lymphoma, non-Hodgkin's lymphoma, diffuse large B-cell lymphoma (DLBCL), B-cell lymphoma, T-cell lymphoma, mantle cell lymphoma, follicular lymphoma (FL), Waldenstrom's macroglobulinemia.

The compound of the present invention can be used to achieve a beneficial therapeutic or prophylactic effect, for example, in subjects with cancer.

In addition, the compounds of the present invention (e.g., a compound of any of the examples as described herein) can be administered in combination with additional therapeutic agents for the treatment of diseases or disorders described herein, such as cancer. The additional therapeutic agents may be administered separately with the compound of the present invention or included with such an ingredient in a pharmaceutical composition according to the disclosure, such as a fixed-dose combination drug product. In some embodiments, additional therapeutic agents are those that are known or discovered to be effective in the treatment of diseases mediated by menin-MLL interaction or at least in part by menin-MLL interaction, such as another menin inhibitor or a compound active against another target associated with the particular disease. The combination may serve to increase efficacy (e.g., by including in the combination a compound potentiating the potency or effectiveness of the compound of the present invention), decrease one or more side effects, or decrease the required dose of the compound of the present invention.

In some embodiments, the compounds of the present invention (e.g., a compound of any of the examples as described herein) can be administered in combination with additional therapeutic agents, such as anti-neoplastic active agents, anti-inflammatory agents, or immunomodulators, wherein the anti-neoplastic active agents include chemotherapeutic agents, immune checkpoint inhibitors or agonists, and targeted therapeutic agents. The term “anti-neoplastic active agent” as used herein refers to any agent that is administered to a subject suffering from cancer for the purposes of treating the cancer, includes, but is not limited to, a chemotherapeutic agent, an immune checkpoint inhibitor or agonist, and a targeted therapeutic agent.

Non-limiting examples of chemotherapeutic agents include topoisomerase I inhibitors (e.g., irinotecan, topotecan, camptothecin and analogs or metabolites thereof, and doxorubicin); topoisomerase II inhibitors (e.g., etoposide, teniposide, mitoxantrone, idarubicin, and daunorubicin); alkylating agents (e.g., melphalan, chlorambucil, busulfan, thiotepa, ifosfamide, carmustine, lomustine, semustine, streptozocin, decarbazine, methotrexate, mitomycin C, and cyclophosphamide); DNA intercalators (e.g., cisplatin, oxaliplatin, and carboplatin); free radical generators such as bleomycin; nucleoside mimetics (e.g., 5-fluorouracil, capecitabine, gemcitabine, fludarabine, cytarabine, azacitidine, mercaptopurine, thioguanine, pentostatin, and hydroxyurea); paclitaxel, docetaxel, and related analogs; vincristine, vinblastin, and related analogs; thalidomide and related analogs (e.g., CC-5013 and CC-4047).

Non-limiting examples of immune checkpoint inhibitors or agonists include PD-1 inhibitors, for example, anti-PD-1 antibodies, such as pembrolizumab, nivolumab, and PDR001 (spartalizumab); PD-L1 inhibitors, for example, anti-PD-L1 antibodies, such as atezolizumab, durvalumab, and avelumab; CTLA-4 inhibitors, such as anti-CTLA-4 antibodies, for example ipilimumab; and BTLA inhibitors, LAG-3 inhibitors, TIM3 inhibitors, TIGIT inhibitors, VISTA inhibitors, OX-40 agonists, and the like.

Targeted therapeutic agents include various small molecule or macromolecular targeted therapeutic agents, and non-limiting examples thereof include: protein tyrosine kinase inhibitors (such as imatinib mesylate and gefitinib); proteasome inhibitors (such as bortezomib); NF-κB inhibitors, including IκB kinase inhibitors; KRAS G12C inhibitors; KRAS G12D inhibitors; ERK inhibitors; CDK4/6 inhibitors; PI3Kδ inhibitors; SYK inhibitors; Bcl-2 inhibitors; BTK inhibitors; EZH1/2 inhibitors; BRAF inhibitors (such as dabrafenib); MEK inhibitors (such as trametinib); mTOR inhibitors (such as rapamycin); anti-CD40 antibodies (such as APX005M, RO7009789); antibodies that bind to proteins overexpressed in cancer to down-regulate cell replication, such as anti-CD20 antibodies (such as rituximab, ibritumomab tiuxetan, and tositumomab), anti-Her2 monoclonal antibodies (such as trastuzumab), anti-EGFR antibodies (such as cetuximab) and anti-VEGF antibodies (such as bevacizumab); anti-angiogenic drugs, such as lenalidomide; and other protein or enzyme inhibitors, these proteins or enzymes are known to be upregulated, overexpressed or activated in cancers, and the inhibition of which can down-regulate cell replication.

### EXAMPLES

The examples below are intended to be purely exemplary and should not be considered to be limiting in any way. Efforts have been made to ensure the accuracy with respect to numbers used (for example, amounts, temperature, etc.), but those skilled in the art should understand that some experimental errors and deviations should be accounted for. Unless indicated otherwise, parts are parts by weight, temperature is in degrees Centigrade, and pressure is at or near atmospheric. All MS data were determined by Agilent 6120 or Agilent 1100. All NMR data were generated using a Varian 400 MHz NMR machine. All reagents, except specific synthesized intermediates, used in the present invention are commercially available. All compound names except the reagents are generated by Chemdraw 19.0. The flash column chromatography is performed using a conventional silica gel column, unless specified otherwise or inconsistent with the context.

If there is any atom with empty valence(s) in any one of the structures disclosed herein, the empty balance(s) is (are) the hydrogen atom(s) which is (are) omitted for convenience purpose.

In the present application, in the case of inconsistency of the name and structure of a compound, when the two of which are both given for the compound, it is subject to the structure of the compound, unless the context shows that the structure of the compound is incorrect and the name is correct.

List of abbreviations used in the following examples:

| | |
|---|---|
| AcOH | Acetic acid |
| Ac₂O | Acetic anhydride |
| AcOK | Potassium acetate |
| BBr₃ | Boron tribromide |
| BINAP | (±)-2,2′-bis-(diphenylphosphino)-1,1′- |
| | binaphthyl |
| Bis(pinacolato)diboron | Bis(pinacolato)diboron |
| (Boc)₂O | Di-tert-butyl dicarbonate |
| Brine | Saturated sodium chloride solution |
| CDI | N,N′-carbonyl diimidazole |
| CeCl₃ | Cerium trichloride |
| Cs₂CO₃ | Cesium carbonate |
| DBU | 1,8-diazabicyclo[5.4.0]undec-7-ene |
| DCM | Dichloromethane |
| DIAD | Diisopropyl azodicarboxylate |
| DIBAL-H | Diisobutylaluminium hydride |
| DIEA | N,N-diisopropylethylamine |
| DMA | N,N-dimethylacetylamide |
| DMAP | 4-dimethylaminopyridine |
| DMF | N,N-dimethylformamide |
| DMP | Dess-Martin periodinane |
| DMSO | Dimethyl sulfoxide |
| EA | Ethyl acetate |
| EtOH | Ethanol |
| Et₃N | Triethylamine |
| HATU | 2-(7-azobenzotriazole)-N,N,N′,N′- |
| | tetramethyluronium hexafluorophosphate |
| LiAlH₄ | Lithium aluminum hydride |
| LiOH —H₂O | Lithium hydroxide monohydrate |
| Mel | Iodomethane |
| MeOH | Methanol |
| MeONa | Sodium methoxide |
| MsCl | Methanesulfonyl chloride |
| NaBD₃CN | d3-Sodium cyanoborodeuteride |
| NaBH₃CN | Sodium cyanoborohydride |
| NaBH(OAc)₃ | Sodium triacetoxyborohydride |
| NIS | N-iodosuccinimide |
| NaOt-Bu | Sodium tert-butoxide |
| Pd/C | Palladium carbon |
| Pd₂(dba)₃ | Tris(dibenzylidene acetone)dipalladium |
| Pd(dppf)Cl₂ —CH₂Cl₂ | [1,1′-bis(diphenylphosphino)ferrocene]palladium |
| | dichloride dichloromethane complex |
| Pd(OAc)₂ | Palladium acetate |
| Pd(PPh₃)₄ | Tetra(triphenylphosphine)palladium |
| PE | Petroleum ether |
| PPh₃ | Triphenylphosphine |
| Py | Pyridine |
| Raney Ni | Raney nickel |
| TBAF | Tetrabutylammonium fluoride |
| TCEP | Tris(2-carboxylethyl)phosphine hydrochloride |
| TEA | Triethylamine |
| TFA | Trifluoroacetic acid |
| THF | Tetrahydrofuran |
| Xantphos | 4,5-bis(diphenylphosphino)-9,9-dimethylxanthene |
| Xphos | 2-dicyclohexylphosphino-2′,4′,6′- |
| | triisopropylbiphenyl |
| Zn(CN)₂ | Zinc cyanide |

### Example 1

### Preparation of Intermediates

### Intermediate 1-1: tert-butyl 2-(3,6-dichloro-1,2,4-triazin-5-yl)-2,7-diazaspiro[3.5]nonane-7-carboxylate

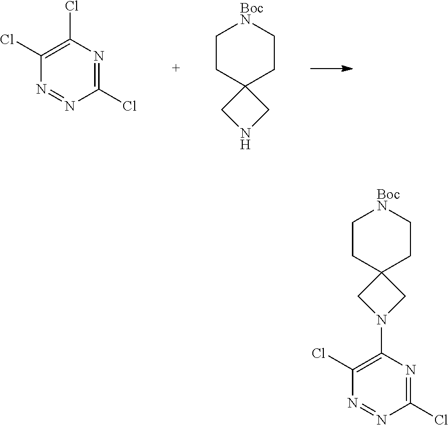 [SMILES: Clc1nnc(Cl)c(Cl)n1 ; CC(C)(C)OC(=O)N1CCC2(CC1)CNC2 ; CC(C)(C)OC(=O)N1CCC2(CC1)CN(c1nc(Cl)nnc1Cl)C2]

In a reaction flask, 3,5,6-trichloro-1,2,4-triazine (5.0 g, 27.0 mmol) and triethylamine (5.46 g, 54 mmol) were dissolved in dichloromethane (30 ml), and tert-butyl 2,7-diazaspiro[3.5]nonane-7-carboxylate (5.52 g, 24.0 mmol) dissolved in 20 ml of dichloromethane was added slowly to the flask under an ice-bath. The mixture was warmed to room temperature and stirred for 1 hour. After the reaction was completed, the reaction solution was washed successively with water and a saturated sodium chloride solution, and subjected to liquid separation. The organic phase was dried over sodium sulfate and then concentrated, and the residue was purified with flash column chromatography (eluted with a gradient of petroleum ether/ethyl acetate=100:0-0:100) to obtain 7.07 g of yellow solid. MS (m/z): 374.2, 376.2 [M+H]+.

The following intermediates were prepared according to the preparation procedure of intermediate 1-1 using corresponding starting materials and reagents under appropriate conditions that will be recognized by those skilled in the art.

| Intermediate | Structural formula | MS (M + H)⁺ |
|---|---|---|
| 1-2 | 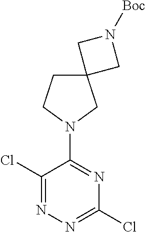 [SMILES: CC(C)(C)OC(=O)N1CC2(CCN(c3nc(Cl)nnc3Cl)C2)C1] | 360.4, 362.4 |
| 1-3 | 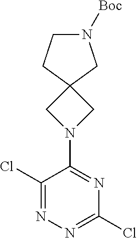 [SMILES: CC(C)(C)OC(=O)N1CCC2(C1)CN(c1nc(Cl)nnc1Cl)C2] | 360.4, 362.4 |
| 1-4 | 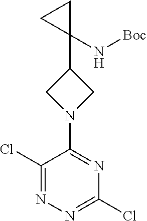 [SMILES: CC(C)(C)OC(=O)NC1(C2CN(c3nc(Cl)nnc3Cl)C2)CC1] | 360.1 |
| 1-5 | 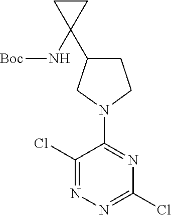 [SMILES: CC(C)(C)OC(=O)NC1(C2CCN(c3nc(Cl)nnc3Cl)C2)CC1] | 374.1 |
| 1-6 | 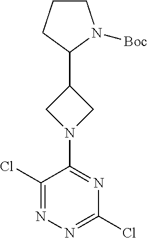 [SMILES: CC(C)(C)OC(=O)N1CCCC1C1CN(c2nc(Cl)nnc2Cl)C1] | 374.2 |

### Intermediate 2-1: N-ethyl-5-fluoro-2-hydroxy-N-isopropylbenzamide

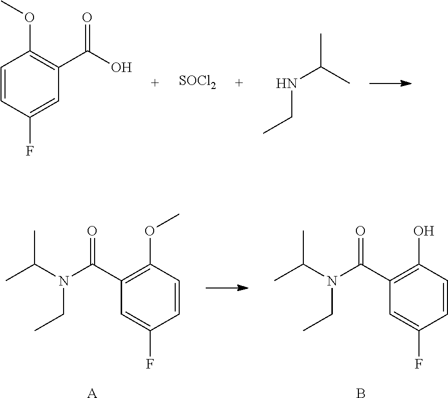

### (A) N-ethyl-5-fluoro-N-isopropyl-2-methoxybenzamide

To a reaction flask were added thionyl chloride (20 ml) and then 5-fluoro-2-methoxybenzoic acid (5.1 g, 30.0 mmol). The mixture was warmed to 80° C. under stirring, and then reacted for 2 hours. The reaction solution was concentrated to remove the solvent, and the residue was dissolved in dichloromethane (100 ml). The solution was cooled to 0° C., and triethylamine (4.55 g, 45.0 mmol) and N-ethylpropan-2-amine (3.14 g, 36.0 mmol) were added. The mixture was warmed to room temperature, then stirred and reacted for 2 hours. After the reaction was completed, the reaction solution was concentrated to remove the solvent. The residue was purified with flash column chromatography (eluted with a gradient of petroleum ether/ethyl acetate=100:0-0:100) to obtain 6.6 g of yellow solid. MS (m/z): 240.0 [M+H]⁺.

### (B) N-ethyl-5-fluoro-2-hydroxy-N-isopropylbenzamide

In a reaction flask, N-ethyl-5-fluoro-N-isopropyl-2-methoxybenzamide (6.6 g, 27.58 mmol) was dissolved in dichloromethane (150 ml). The mixture was cooled to 0° C., and then boron tribromide (33 ml, 36.0 mmol) was added. The resulting mixture was warmed to room temperature, then stirred and reacted for 48 hours. The reaction solution was concentrated to remove the solvent to obtain 5.4 g of yellow solid. MS (m/z): 226.0 [M+H]⁺.

The following intermediates were prepared according to the preparation procedure of intermediate 2-1 using corresponding starting materials and reagents under appropriate conditions that will be recognized by those skilled in the art.

| Intermediate | Structural formula | MS (M + H)⁺ |
|---|---|---|
| 2-2 | 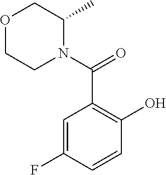 [SMILES: C[C@H]1COCCN1C(=O)c1cc(F)ccc1O] | 240.0 |
| 2-3 | 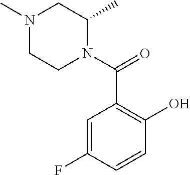 [SMILES: C[C@H]1CN(C)CCN1C(=O)c1cc(F)ccc1O] | 253.2 |
| 2-4 | 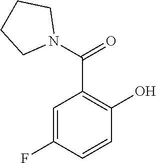 [SMILES: O=C(c1cc(F)ccc1O)N1CCCC1] | 210.0 |
| 2-5 | 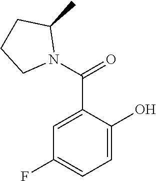 [SMILES: C[C@@H]1CCCN1C(=O)c1cc(F)ccc1O] | 224.2 |

### Intermediate 3: isopropyl 5-fluoro-2-hydroxybenzoate

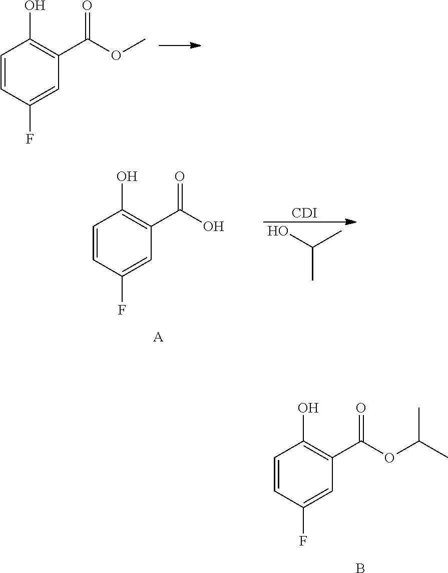

### (A) 5-fluoro-2-hydroxybenzoic acid

To a reaction flask were added methyl 5-fluoro-2-hydroxybenzoate (3.0 g, 17.63 mmol), sodium hydroxide (2.12 g, 52.90 mmol), methanol (30 ml) and water (10 ml), and the mixture was heated to 60° C. and reacted for 15 hours. The reaction solution was cooled to room temperature, adjusted to pH=2.0 with 1 mole/liter dilute hydrochloric acid and filtered, and the filter cake was dried to obtain 2.6 g of white solid. MS (m/z): 157.1 [M+H]⁺.

### (B) isopropyl 5-fluoro-2-hydroxybenzoate

To a reaction flask were added 5-fluoro-2-hydroxybenzoic acid (2.6 g, 16.65 mmol), N,N′-carbonyl diimidazole (2.7 g, 16.65 mmol) and N,N-dimethylformamide (10 ml), and the mixture was heated to 50° C. and reacted for 3 hours. Isopropanol (5 ml) was then added, and the resulting mixture was heated to 50° C. and reacted for 15 hours. After the reaction was completed, the reaction solution was concentrated, and the residue was purified with flash column chromatography (eluted with a gradient of PE/EA=100:0-0:100) to obtain 2.37 g of colorless oil.

¹H NMR (400 MHz, CD₃OD) δ 7.43 (d, J=8.4 Hz, 1H), 7.20 (s, 1H), 6.89 (s, 1H), 5.37-5.13 (m, 1H), 1.36 (d, J=8.0, 6.4 Hz, 6H).

### Intermediate 4: N-ethyl-5-fluoro-2-hydroxy-N-isopropylbenzothioamide

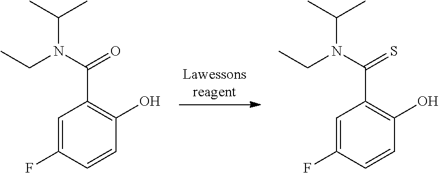 [SMILES: CCN(C(=O)c1cc(F)ccc1O)C(C)C ; CCN(C(=S)c1cc(F)ccc1O)C(C)C]

To a reaction flask were added N-ethyl-5-fluoro-2-hydroxy-N-isopropylbenzamide (500 mg, 2.22 mmol), Lawesson's reagent (538 mg, 1.33 mmol) and toluene (20 ml), and the mixture was heated to reflux, and reacted for 10 hours. After the reaction was completed, the reaction solution was concentrated, and the residue was purified with flash column chromatography (eluted with a gradient of water/methanol=100:0-0:100) to obtain 450 mg of white solid. MS (m/z): 242.1 [M+H]⁺.

### Intermediate 5-1: 4-fluoro-2-(1-isopropyl-1H-pyrazol-5-yl)phenol

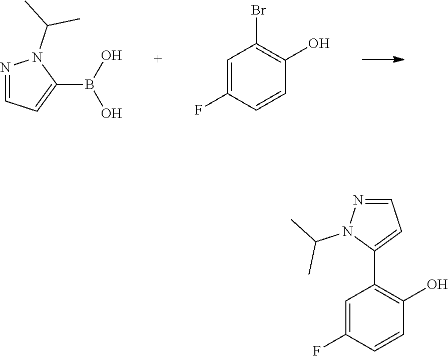 [SMILES: CC(C)n1nccc1B(O)O ; Oc1ccc(F)cc1Br ; CC(C)n1nccc1-c1cc(F)ccc1O]

To a reaction flask were added successively (1-isopropyl-1H-pyrazol-5-yl)boronic acid (330 mg, 2.14 mmol), 2-bromo-4-fluorophenol (341 mg, 1.79 mmol), Pd(dppf)Cl₂·CH₂Cl₂ (147 mg, 0.18 mmol), potassium carbonate (50 mg, 0.36 mmol), dioxane (25 ml) and water (5 ml), and the mixture was heated to 100° C. and reacted for hours under nitrogen protection. After the reaction was completed, the reaction solution was concentrated, and the residue was purified with flash column chromatography (eluted with a gradient of water/methanol=100:0-0:100) to obtain 140 mg of yellow solid. MS (m/z): 221.4 [M+H]⁺.

The following intermediates were prepared according to the preparation procedure of intermediate 5-1 using corresponding starting materials and reagents under appropriate conditions that will be recognized by those skilled in the art.

| Intermediate | Structural formula | MS (M + H)⁺ |
|---|---|---|
| 5-2 | 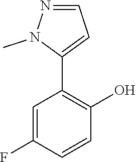 | 193.0 |
| 5-3 | 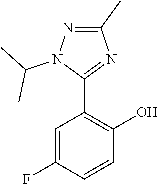 [SMILES: Cc1nc(-c2cc(F)ccc2O)n(C(C)C)n1] | 236.4 |
| 5-4 | 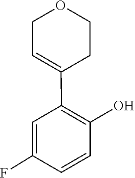 [SMILES: Oc1ccc(F)cc1C1=CCOCC1] | 195.4 |
| 5-5 | 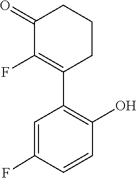 [SMILES: O=C1CCCC(c2cc(F)ccc2O)=C1F] | 225.2 |

### Intermediate 6-1: 7-fluoro-1-isopropyl-1H-indazol-4-ol

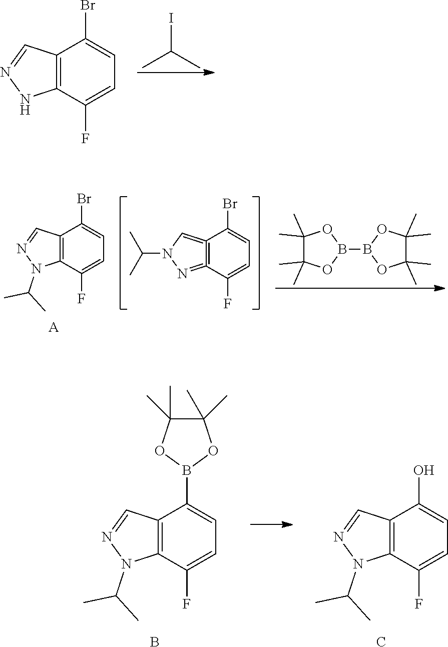

### (A) 4-bromo-7-fluoro-1-isopropyl-1H-indazole

To a reaction flask were added 4-bromo-7-fluoro-1H-indazole (1.5 g, 6.98 mmol) and anhydrous tetrahydrofuran (20 ml), and the mixture was cooled to 0° C. Sodium hydride (60%, 560 mg, 13.96 mmol) was added in batches, and the mixture was stirred at 0° C. for additional 30 minutes. Iodoisopropane (1.423 g, 8.37 mmol) was added, and then the resulting mixture was stirred at room temperature for 15 hours. After the reaction was completed, the reaction solution was quenched with water, and extracted with ethyl acetate. The organic phase was concentrated, and the residue was purified with flash column chromatography (eluted with a gradient of petroleum ether/ethyl acetate=100:0-0:100) to obtain 1.2 g of 4-bromo-7-fluoro-1-isopropyl-1H-indazole (MS (m/z): 257.2, 259.2 [M+H]⁺) and 600 mg of 4-bromo-7-fluoro-2-isopropyl-2H-indazole (MS (m/z): 257.2, 259.2 [M+H]⁺).

### (B) 7-fluoro-1-isopropyl-4-(4,4,5,5-tetramethyl-1,3,2-dioxaborolan-2-yl)-1H-indazole

To a reaction flask were added successively 4-bromo-7-fluoro-1-isopropyl-1H-indazole (1.2 g, 4.67 mmol), bis(pinacolato)diboron (1.78 g, 7.01 mmol), Pd(dppf)Cl₂·CH₂Cl₂ (377 mg, 0.46 mmol), potassium acetate (1.37 g, 13.96 mmol) and dioxane (30 ml), and the mixture was heated to 100° C. and reacted for 15 hours under nitrogen protection. After the reaction was completed, the reaction solution was concentrated, and the residue was purified with flash column chromatography (eluted with a gradient of water/methanol=100:0-0:100) to obtain 1.13 g of yellow solid. MS (m/z): 305.4 [M+H]⁺.

### (C) 7-fluoro-1-isopropyl-1H-indazol-4-ol

To a reaction flask were added 7-fluoro-1-isopropyl-4-(4,4,5,5-tetramethyl-1,3,2-dioxaborolan-2-yl)-1H-indazole (1.13 g, 3.72 mmol), 1 mole/liter aqueous sodium hydroxide solution (3.72 ml, 3.72 mmol) and methanol (15 ml). Hydrogen peroxide (0.5 ml) was added dropwise at room temperature, and the mixture was stirred for 1 hour. After the reaction was completed, ethyl acetate was added, and the mixture was washed with a saturated sodium sulfite solution and saturated brine. The organic phase was concentrated, and the residue was purified with flash column chromatography (eluted with a gradient of petroleum ether/ethyl acetate=100:0-0:100) to obtain 600 mg of white solid. MS (m/z): 195.1 [M+H]⁺.

The following intermediates were prepared according to the preparation procedure of intermediate 6-1 using corresponding starting materials and reagents under appropriate conditions that will be recognized by those skilled in the art.

| Intermediate | Structural formula | MS (M + H)⁺ |
|---|---|---|
| 6-2 | 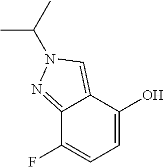 | 195.1 |

### Intermediate 7-1: 2-((5-(2,7-diazaspiro[3.5]nonan-2-yl)-1,2,4-triazin-6-yl)oxy)-N-ethyl-5-fluoro-N-isopropylbenzamide

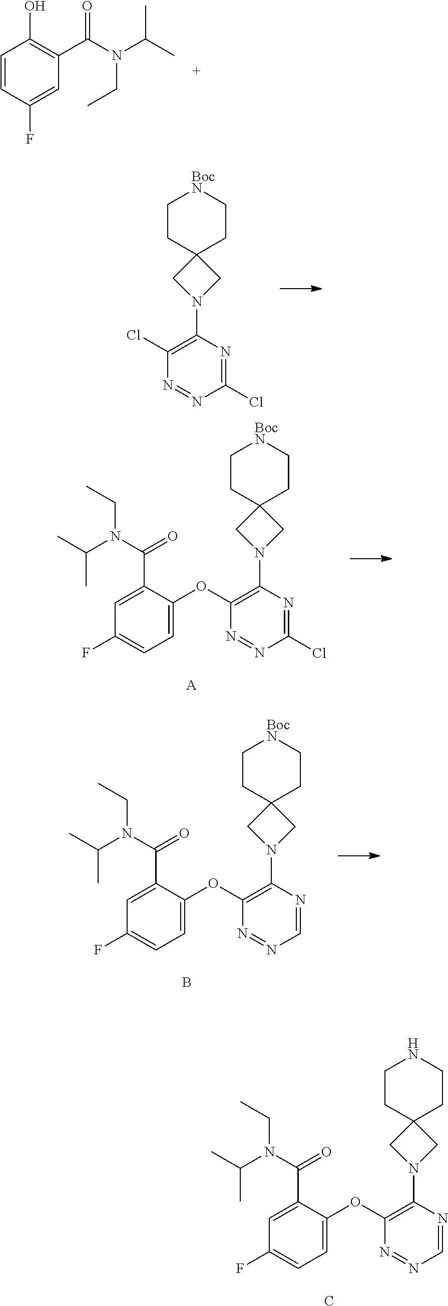

### (A) tert-butyl 2-(3-chloro-6-(2-(ethyl(isopropyl)carbamoyl)-4-fluorophenoxy)-1,2,4-triazin-5-yl)-2,7-diazaspiro[3.5]nonane-7-carboxylate

To a reaction flask were added successively tert-butyl 2-(3,6-dichloro-1,2,4-triazin-5-yl)-2,7-diazaspiro[3.5]nonane-7-carboxylate (7.07 g, 18.9 mmol), N-ethyl-5-fluoro-2-hydroxy-N-isopropylbenzamide (4.26 g, 18.9 mmol), DBU (9.50 g, 62.4 mmol) and tetrahydrofuran (50 ml), and the mixture was warmed to 40° C., then stirred and reacted overnight. The reaction solution was diluted with water, and extracted three times with ethyl acetate. The ethyl acetate phase was washed successively with water and saturated sodium chloride, and then dried over sodium sulfate. After drying, the solution was concentrated to remove the solvent, and the residue was purified with flash column chromatography (eluted with a gradient of petroleum ether/ethyl acetate=100:0-0:100) to obtain 7.55 g of yellow solid. MS (m/z): 563.4 [M+H]⁺.

### (B) tert-butyl 2-(6-(2-(ethyl(isopropyl)carbamoyl)-4-fluorophenoxy)-1,2,4-triazin-5-yl)-2,7-diazaspiro[3.5]nonane-7-carboxylate

To a reaction flask were added successively tert-butyl 2-(3-chloro-6-(2-(ethyl(isopropyl)carbamoyl)-4-fluorophenoxy)-1,2,4-triazin-5-yl)-2,7-diazaspiro[3.5]nonane-7-carboxylate (7.55 g, 13.4 mmol), palladium carbon (1 g) and methanol (100 ml). The air is replaced with a hydrogen balloon, and the mixture was stirred and reacted for 15 hours at room temperature. The reaction solution was filtered to remove palladium carbon and concentrated. The residue was purified with flash column chromatography (eluted with a gradient of petroleum ether/ethyl acetate=100:0-0:100) to obtain 4.8 g of yellow solid. MS (m/z): 529.4 [M+H]⁺.

### (C) 2-((5-(2,7-diazaspiro[3.5]nonan-2-yl)-1,2,4-triazin-6-yl)oxy)-N-ethyl-5-fluoro-N-isopropylbenzamide

To a reaction flask were added successively tert-butyl 2-(6-(2-(ethyl(isopropyl) carbamoyl)-4-fluorophenoxy)-1,2,4-triazin-5-yl)-2,7-diazaspiro[3.5]nonane-7-carboxylate (4.8 g, 9.1 mmol), concentrated hydrochloric acid (10 ml) and methanol (100 ml), and the mixture was stirred and reacted for 1 hour at room temperature. The reaction solution was concentrated to remove the solvent, and then the residue was dissolved in dichloromethane. The resulting solution was washed successively with saturated sodium carbonate and saturated sodium chloride, dried over sodium sulfate, and then concentrated to obtain 3.74 g of white solid. MS (m/z): 429.2 [M+H]⁺.

The following intermediates were prepared according to the preparation procedure of intermediate 7-1 using corresponding starting materials and reagents under appropriate conditions that will be recognized by those skilled in the art.

| Inter- | | MS |
|---|---|---|
| mediate | Structural formula | (M + H)⁺ |
| 7-2 | 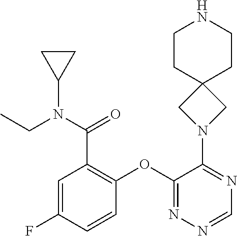 [SMILES: CCN(C(=O)c1cc(F)ccc1Oc1nncnc1N1CC2(CCNCC2)C1)C1CC1] | 527.3 |
| 7-3 | 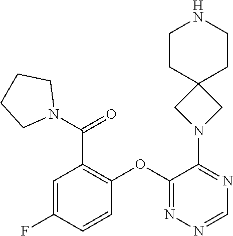 [SMILES: O=C(c1cc(F)ccc1Oc1nncnc1N1CC2(CCNCC2)C1)N1CCCC1] | 413.2 |
| 7-4 | 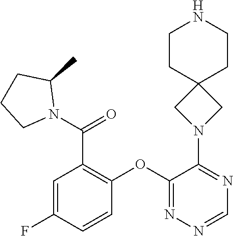 [SMILES: C[C@@H]1CCCN1C(=O)c1cc(F)ccc1Oc1nncnc1N1CC2(CCNCC2)C1] | 427.2 |
| 7-5 | 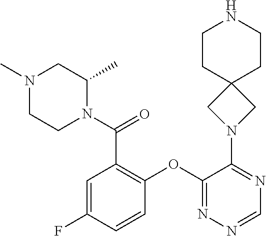 [SMILES: C[C@H]1CN(C)CCN1C(=O)c1cc(F)ccc1Oc1nncnc1N1CC2(CCNCC2)C1] | 456.3 |
| 7-6 | 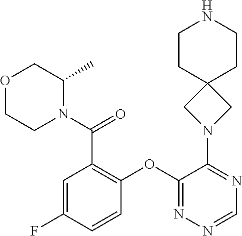 [SMILES: C[C@H]1COCCN1C(=O)c1cc(F)ccc1Oc1nncnc1N1CC2(CCNCC2)C1] | 443.2 |
| 7-7 | 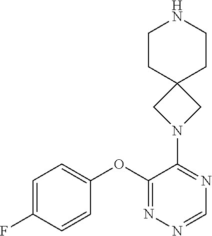 [SMILES: Fc1ccc(Oc2nncnc2N2CC3(CCNCC3)C2)cc1] | 316.2 |
| 7-8 | 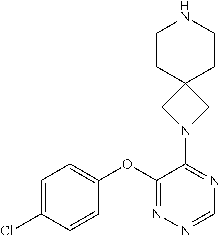 [SMILES: Clc1ccc(Oc2nncnc2N2CC3(CCNCC3)C2)cc1] | 332.1 |
| 7-9 | 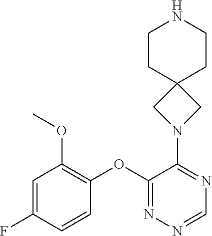 [SMILES: COc1cc(F)ccc1Oc1nncnc1N1CC2(CCNCC2)C1] | 346.1 |
| 7-10 | 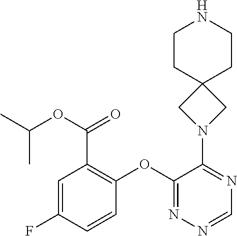 [SMILES: CC(C)OC(=O)c1cc(F)ccc1Oc1nncnc1N1CC2(CCNCC2)C1] | 402.6 |
| 7-11 | 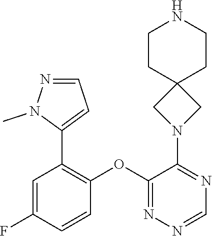 [SMILES: Cn1nccc1-c1cc(F)ccc1Oc1nncnc1N1CC2(CCNCC2)C1] | 396.2 |
| 7-12 | 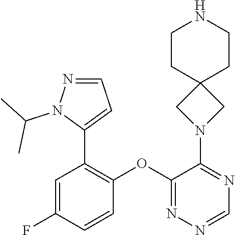 [SMILES: CC(C)n1nccc1-c1cc(F)ccc1Oc1nncnc1N1CC2(CCNCC2)C1] | 424.4 |
| 7-13 | 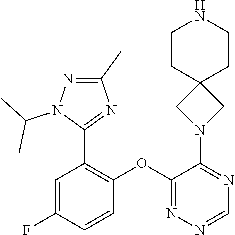 [SMILES: Cc1nc(-c2cc(F)ccc2Oc2nncnc2N2CC3(CCNCC3)C2)n(C(C)C)n1] | 439.6 |
| 7-14 | 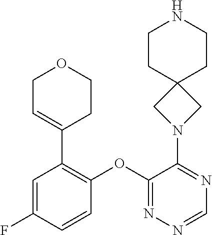 [SMILES: Fc1ccc(Oc2nncnc2N2CC3(CCNCC3)C2)c(C2=CCOCC2)c1] | 398.6 |
| 7-15 | 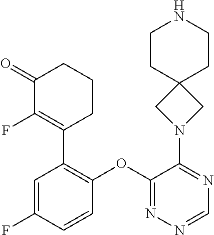 [SMILES: O=C1CCCC(c2cc(F)ccc2Oc2nncnc2N2CC3(CCNCC3)C2)=C1F] | 428.0 |
| 7-16 | 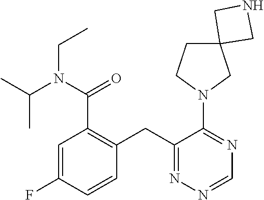 [SMILES: CCN(C(=O)c1cc(F)ccc1Cc1nncnc1N1CCC2(CNC2)C1)C(C)C] | 415.4 |
| 7-17 | 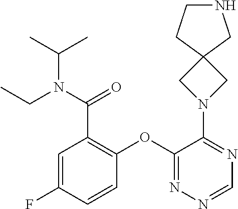 [SMILES: CCN(C(=O)c1cc(F)ccc1Oc1nncnc1N1CC2(CCNC2)C1)C(C)C] | 415.4 |
| 7-18 | 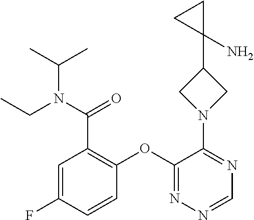 [SMILES: CCN(C(=O)c1cc(F)ccc1Oc1nncnc1N1CC(C2(N)CC2)C1)C(C)C] | 415.2 |
| 7-19 | 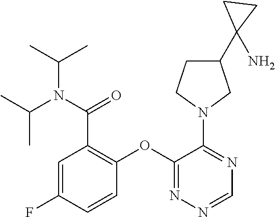 [SMILES: CC(C)N(C(=O)c1cc(F)ccc1Oc1nncnc1N1CCC(C2(N)CC2)C1)C(C)C] | 429.2 |
| 7-20 | 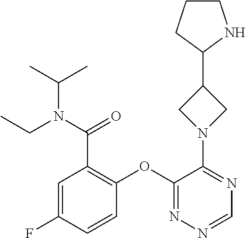 [SMILES: CCN(C(=O)c1cc(F)ccc1Oc1nncnc1N1CC(C2CCCN2)C1)C(C)C] | 429.4 |
| 7-21 | 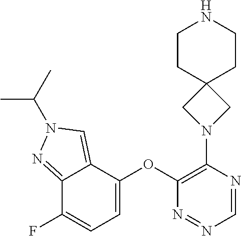 [SMILES: CC(C)n1cc2c(Oc3nncnc3N3CC4(CCNCC4)C3)ccc(F)c2n1] | 398.2 |
| 7-22 | 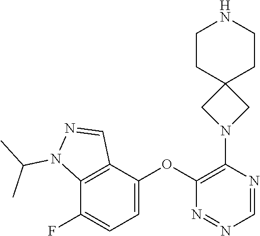 [SMILES: CC(C)n1ncc2c(Oc3nncnc3N3CC4(CCNCC4)C3)ccc(F)c21] | 398.2 |
| 7-23 | 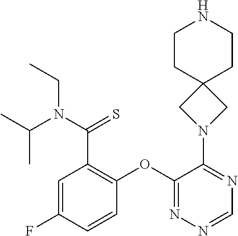 [SMILES: CCN(C(=S)c1cc(F)ccc1Oc1nncnc1N1CC2(CCNCC2)C1)C(C)C] | 445.2 |
| 7-24 | 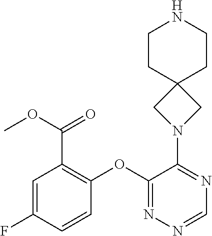 [SMILES: COC(=O)c1cc(F)ccc1Oc1nncnc1N1CC2(CCNCC2)C1] | 374.1 |
| 7-25 | 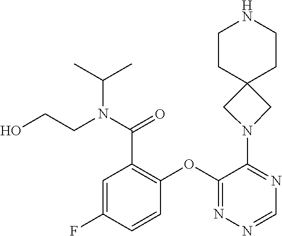 [SMILES: CC(C)N(CCO)C(=O)c1cc(F)ccc1Oc1nncnc1N1CC2(CCNCC2)C1] | 445.2 |
| 7-26 | 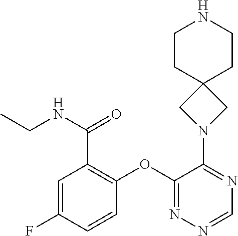 [SMILES: CCNC(=O)c1cc(F)ccc1Oc1nncnc1N1CC2(CCNCC2)C1] | 387.3 |

### Intermediate 8: N-ethyl-5-fluoro-N-isopropyl-2-((3-(methylamino)-5-(2,7-diazaspiro[3.5]nonan-2-yl)-1,2,4-triazin-6-yl)oxy)benzamide

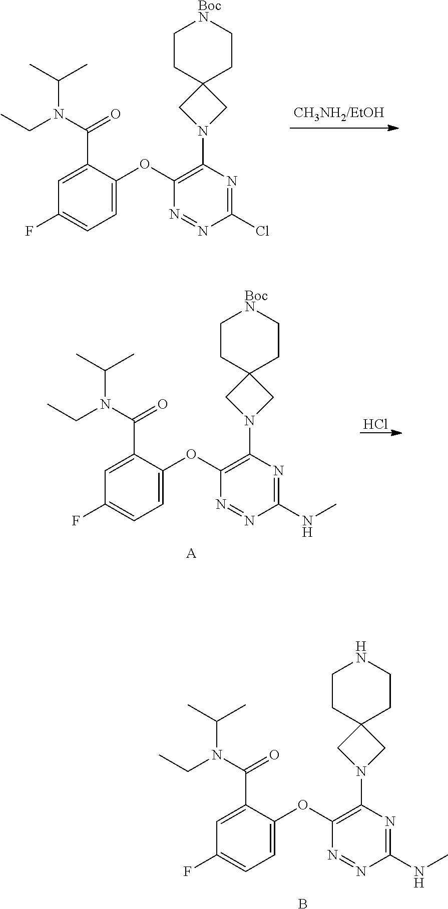

### (A) tert-butyl 2-(6-(2-(ethyl(isopropyl)carbamoyl)-4-fluorophenoxy)-3-(methylamino)-1,2,4-triazin-5-yl)-2,7-diazaspiro[3.5]nonane-7-carboxylate

To a sealed tube were added tert-butyl 2-(3-chloro-6-(2-(ethyl(isopropyl)carbamoyl)-4-fluorophenoxy)-1,2,4-triazin-5-yl)-2,7-diazaspiro[3.5]nonane-7-carboxylate (150 mg, 0.266 mmol) and 30% methylamine in ethanol (2 ml), and the mixture was heated to 90° C., stirred and reacted for 1 hour under sealed conditions. After the reaction was completed, the reaction solution was concentrated, and the residue was purified with flash column chromatography (eluted with a gradient of petroleum ether/ethyl acetate=100:0-0:100) to obtain 80 mg of yellow solid. MS (m/z): 558.3 [M+H]⁺.

### (B) N-ethyl-5-fluoro-N-isopropyl-2-((3-(methylamino)-5-(2,7-diazaspiro[3.5]nonan-2-yl)-1,2,4-triazin-6-yl)oxy)benzamide

To a reaction flask were added tert-butyl 2-(6-(2-(ethyl(isopropyl)carbamoyl)-4-fluorophenoxy)-3-(methylamino)-1,2,4-triazin-5-yl)-2,7-diazaspiro[3.5]nonane-7-carboxylate (80 mg, 0.143 mmol) and 6 mole/liter hydrochloric acid in methanol (5 ml), and then the mixture was stirred and reacted for 2 hours at room temperature. After the reaction was completed, the reaction solution was concentrated, and the residue was dissolved in dichloromethane, and washed with a saturated sodium bicarbonate solution and brine. The organic phase was dried over anhydrous sodium sulfate, and concentrated to obtain 60 mg of white solid. MS (m/z): 458.3 [M+H]⁺.

### Intermediate 9: N-ethyl-5-fluoro-N-isopropyl-2-((3-methyl-5-(2,7-diazaspiro[3.5]nonan-2-yl)-1,2,4-triazin-6-yl)oxy)benzamide

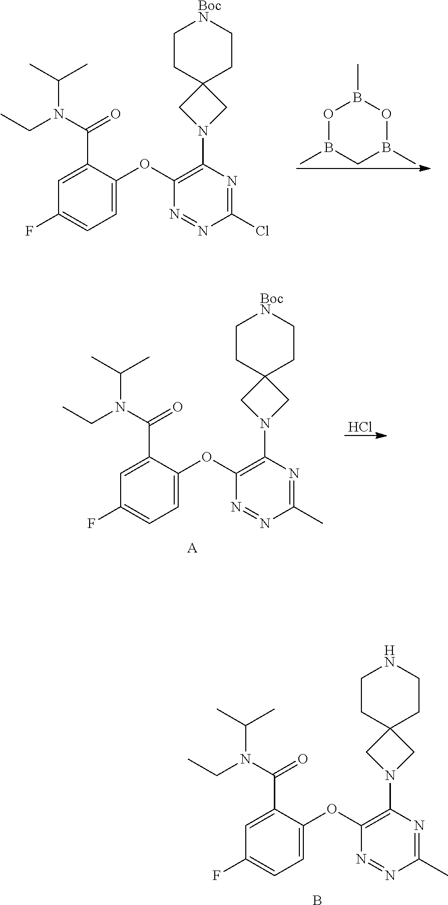

### (A) tert-butyl 2-(6-(2-(ethyl(isopropyl)carbamoyl)-4-fluorophenoxy)-3-methyl-1,2,4-triazin-5-yl)-2,7-diazaspiro[3.5]nonane-7-carboxylate

To a reaction flask were added tert-butyl 2-(3-chloro-6-(2-(ethyl(isopropyl) carbamoyl)-4-fluorophenoxy)-1,2,4-triazin-5-yl)-2,7-diazaspiro[3.5]nonane-7-carboxylate (200 mg, 0.355 mmol), trimethylboroxine (178 mg, 1.421 mmol), tetra(triphenylphosphine)palladium (41 mg, 0.0355 mmol), potassium carbonate (98 mg, 0.71 mmol) and dioxane (20 ml), and the mixture was heated to 110° C., stirred and reacted for 15 hours under nitrogen protection. After the reaction was completed, the reaction solution was concentrated, and the residue was purified with flash column chromatography (eluted with a gradient of water/methanol=100:0-0:100) to obtain 150 mg of white solid. MS (m/z): 543.6 [M+H]⁺.

### (B) N-ethyl-5-fluoro-N-isopropyl-2-((3-methyl-5-(2,7-diazaspiro[3.5]nonan-2-yl)-1,2,4-triazin-6-yl)oxy)benzamide

To a reaction flask were added tert-butyl 2-(6-(2-(ethyl(isopropyl)carbamoyl)-4-fluorophenoxy)-3-methyl-1,2,4-triazin-5-yl)-2,7-diazaspiro[3.5]nonane-7-carboxylate (150 mg, 0.276 mmol) and 6.0 mole/liter hydrochloric acid in methanol (5 ml), and then the mixture was stirred and reacted for 2 hours at room temperature. After the reaction was completed, the reaction solution was concentrated, and the residue was dissolved in dichloromethane, and washed with a saturated sodium bicarbonate solution and brine. The organic phase was dried over anhydrous sodium sulfate, and concentrated to obtain 120 mg of white solid. MS (m/z): 443.3 [M+H]⁺.

### Intermediate 10-1: N-ethyl-5-fluoro-N-isopropyl-2-((5-(7-(piperidin-4-ylmethyl)-2,7-diazaspiro[3.5]nonan-2-yl)-1,2,4-triazin-6-yl)oxy)benzamide

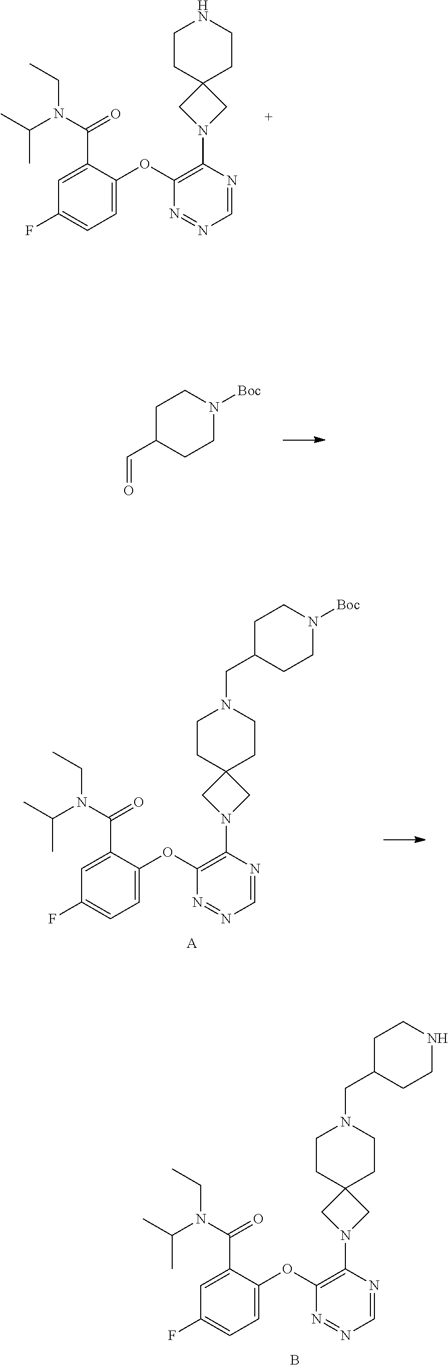

### (A) tert-butyl 4-((2-(6-(2-(ethyl(isopropyl)carbamoyl)-4-fluorophenoxy)-1,2,4-triazin-5-yl)-2,7-diazaspiro[3.5]nonan-7-yl)methyl)piperidine-1-carboxylate

To a reaction flask were added successively 2-((5-(2,7-diazaspiro[3.5]nonan-2-yl)-1,2,4-triazin-6-yl)oxy)-N-ethyl-5-fluoro-N-isopropylbenzamide (400 mg, 0.93 mmol), tert-butyl 4-formylpiperidine-1-carboxylate (298 mg, 1.4 mmol), methanol (10 ml) and acetic acid (1 ml), followed by sodium cyanoborohydride (120 mg, 1.91 mmol). The mixture was reacted at room temperature for 5 hours, and the reaction solution was concentrated to remove the solvent. The residue was then purified with flash column chromatography (eluted with a gradient of water/methanol=100:0-0:100) to obtain 450 mg of solid. MS (m/z): 626.4 [M+H]⁺.

### (B) N-ethyl-5-fluoro-N-isopropyl-2-((5-(7-(piperidin-4-ylmethyl)-2,7-diazaspiro[3.5]nonan-2-yl)-1,2,4-triazin-6-yl)oxy)benzamide

In a reaction flask, tert-butyl 4-((2-(6-(2-(ethyl(isopropyl)carbamoyl)-4-fluorophenoxy)-1,2,4-triazin-5-yl)-2,7-diazaspiro[3.5]nonan-7-yl)methyl)piperidine-1-carboxylate (450 mg, 0.72 mmol) was dissolved in dichloromethane (10 ml), trifluoroacetic acid (4 ml) was added, and the mixture was reacted at room temperature for 5 hours. The reaction solution was concentrated to remove the solvent, and then the residue was dissolved in dichloromethane. The resulting solution was neutralized with saturated sodium carbonate, washed with saturated sodium chloride, dried over sodium sulfate, and then concentrated, and the residue was purified with flash column chromatography (eluted with a gradient of water/methanol=100:0-0:100) to obtain 350 mg of solid. MS (m/z): 526.3 [M+H]⁺.

The following intermediates were prepared according to the preparation procedure of intermediate 10-1 using corresponding starting materials and reagents under appropriate conditions that will be recognized by those skilled in the art.

| | | MS |
|---|---|---|
| Intermediate | Structural formula | (M + H)⁺ |
| 10-2 | 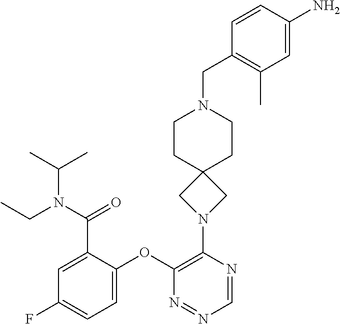 [SMILES: CCN(C(=O)c1cc(F)ccc1Oc1nncnc1N1CC2(CCN(Cc3ccc(N)cc3C)CC2)C1)C(C)C] | 548.9 |
| 10-3 | 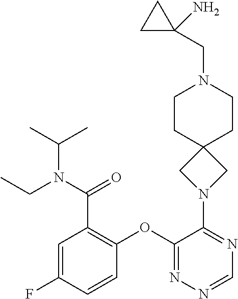 [SMILES: CCN(C(=O)c1cc(F)ccc1Oc1nncnc1N1CC2(CCN(CC3(N)CC3)CC2)C1)C(C)C] | 498.3 |
| 10-4 | 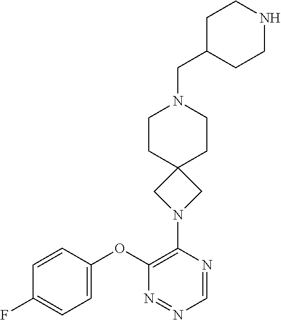 [SMILES: Fc1ccc(Oc2nncnc2N2CC3(CCN(CC4CCNCC4)CC3)C2)cc1] | 413.2 |
| 10-5 | 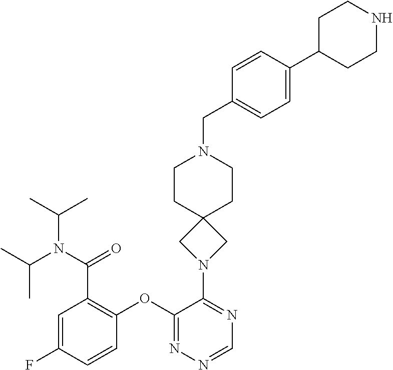 [SMILES: CC(C)N(C(=O)c1cc(F)ccc1Oc1nncnc1N1CC2(CCN(Cc3ccc(C4CCNCC4)cc3)CC2)C1)C(C)C] | 602.4 |
| 10-6 | 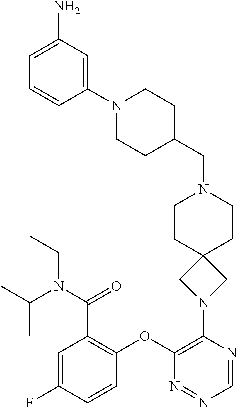 [SMILES: CCN(C(=O)c1cc(F)ccc1Oc1nncnc1N1CC2(CCN(CC3CCN(c4cccc(N)c4)CC3)CC2)C1)C(C)C] | 617.4 |
| 10-7 | 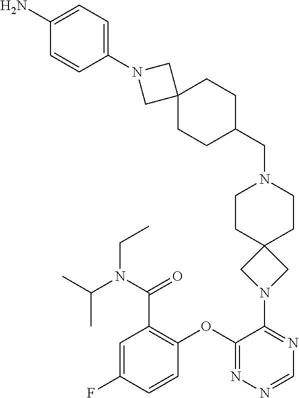 [SMILES: CCN(C(=O)c1cc(F)ccc1Oc1nncnc1N1CC2(CCN(CC3CCC4(CC3)CN(c3ccc(N)cc3)C4)CC2)C1)C(C)C] | 657.4 |
| 10-8 | 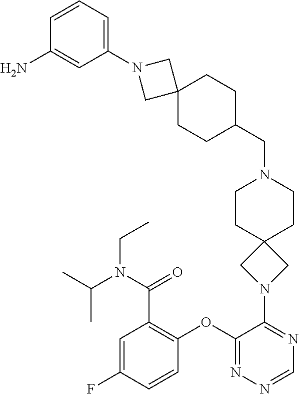 [SMILES: CCN(C(=O)c1cc(F)ccc1Oc1nncnc1N1CC2(CCN(CC3CCC4(CC3)CN(c3cccc(N)c3)C4)CC2)C1)C(C)C] | 657.4 |

### Intermediate 11: 1-(pyridin-2-yl)piperidine-4-carboxylic acid

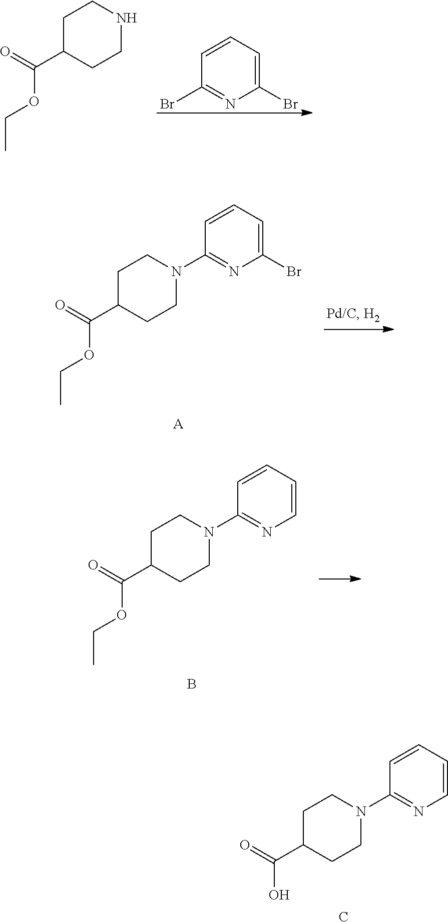

### (A) ethyl 1-(6-bromopyridin-2-yl)piperidine-4-carboxylate

To a reaction flask were added successively ethyl piperidine-4-carboxylate (314 mg, 2.0 mmol), 2,6-dibromopyridine (474 mg, 2.0 mmol), cesium carbonate (975 mg, 3.0 mmol) and dimethyl sulfoxide (5 ml), and the mixture was heated to 100° C. and reacted for 15 hours. The reaction solution was purified with flash column chromatography (eluted with a gradient of water/methanol=100:0-0:100) to obtain 495 mg of white solid. MS (m/z): 313.2 [M+H]⁺.

### (B) ethyl 1-(pyridin-2-yl)piperidine-4-carboxylate

To a reaction flask were added successively ethyl 1-(6-bromopyridin-2-yl)piperidine-4-carboxylate (495 mg, 1.58 mmol), palladium carbon (50 mg) and methanol (10 ml), and the mixture was reacted for 2 hours under hydrogen. After the reaction was completed, the reaction solution was filtered, and the filtrate was concentrated to obtain 400 mg of light yellow solid, which was directly used in the next reaction. MS (m/z): 235.2 [M+H]⁺.

### (C) 1-(pyridin-2-yl)piperidine-4-carboxylic acid

To a reaction flask were added ethyl 1-(pyridin-2-yl)piperidine-4-carboxylate (401 mg, 1.71 mmol), lithium hydroxide monohydrate (199 mg, 4.74 mmol), methanol (8 ml) and water (2 ml), and the mixture was reacted at room temperature for 2 hours. The reaction solution was adjusted to pH 5-6 with 1 mole/liter dilute hydrochloric acid, extracted three times with ethyl acetate, dried over anhydrous sodium sulfate, filtered, and concentrated to obtain 183 mg of white solid, which was directly used in the next reaction. MS (m/z): 207.2 [M+H]⁺.

### Intermediate 12-1: 1-(pyridin-2-yl)piperidine-4-carbaldehyde

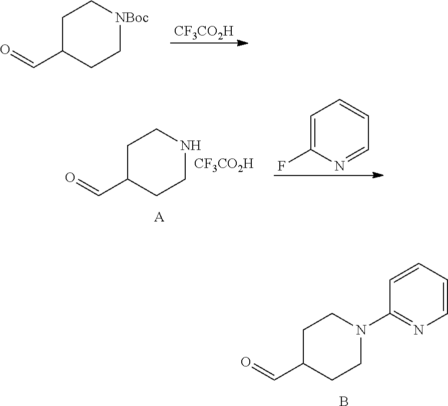

### (A) piperidine-4-carbaldehyde trifluoroacetate

In a reaction flask, tert-butyl 4-formylpiperidine-1-carboxylate (2.13 g, 10.0 mmol) and trifluoroacetic acid (5 ml) were dissolved in dichloromethane (15 ml), and the reaction solution was stirred for 15 hours at room temperature. After the reaction was completed, the reaction solution was concentrated to dryness, to obtain 2.27 g of crude product, which was directly used in the next reaction without further purification. MS (m/z): 146.4 [M+H]⁺.

### (B) 1-(pyridin-2-yl)piperidine-4-carbaldehyde

In a reaction flask, piperidine-4-carbaldehyde trifluoroacetate (454 mg, 2.0 mmol), 2-fluoropyridine (388 mg, 4.0 mmol) and potassium carbonate (553 mg, 4.0 mmol) were dissolved in N,N-dimethylformamide (5 ml), and the reaction solution was heated to reflux for 4 hours. After the reaction was completed, the reaction solution was diluted with water, extracted with ethyl acetate, and then concentrated, and the residue was purified with flash column chromatography (eluted with a gradient of water/methanol=100:0-0:100) to obtain 90 mg of product. MS (m/z): 223.4 [M+OCH₃]⁺.

The following intermediates were prepared according to the preparation procedure of intermediate 12-1 using corresponding starting materials and reagents under appropriate conditions that will be recognized by those skilled in the art.

| | | MS |
|---|---|---|
| Intermediate | Structural formula | (M + H)⁺ |
| 12-2 | 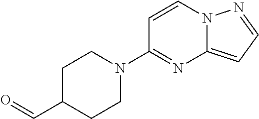 [SMILES: O=CC1CCN(c2ccn3nccc3n2)CC1] | 230.2 |
| 12-3 | 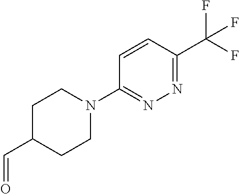 [SMILES: O=CC1CCN(c2ccc(C(F)(F)F)nn2)CC1] | 260.2 |
| 12-4 | 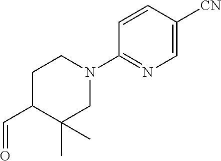 [SMILES: CC1(C)CN(c2ccc(C#N)cn2)CCC1C=O] | 244.2 |
| 12-5 | 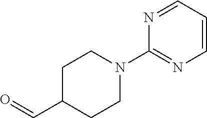 [SMILES: O=CC1CCN(c2ncccn2)CC1] | 192.1 |
| 12-6 | 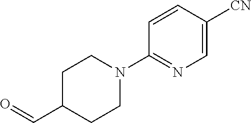 [SMILES: N#Cc1ccc(N2CCC(C=O)CC2)nc1] | 216.2 |
| 12-7 | 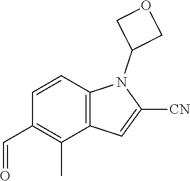 [SMILES: Cc1c(C=O)ccc2c1cc(C#N)n2C1COC1] | 241.1 |

### Intermediate 13-1: 5-formyl-4-methyl-1-(2-((tetrahydro-2H-pyran-2-yl)oxy)ethyl)-1H-indole-2-carbonitrile

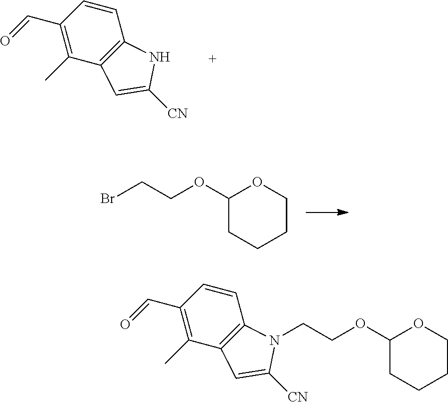 [SMILES: Cc1c(C=O)ccc2[nH]c(C#N)cc12 ; BrCCOC1CCCCO1 ; Cc1c(C=O)ccc2c1cc(C#N)n2CCOC1CCCCO1]

To a reaction flask were added successively 5-formyl-4-methyl-1H-indole-2-carbonitrile (0.50 g, 2.71 mmol), 2-(2-bromoethoxy)tetrahydro-2H-pyrane (0.68 g, 3.25 mmol), cesium carbonate (1.32 g, 4.05 mmol) and N,N-dimethylformamide (10 ml), and the mixture was heated to 50° C. and reacted for 5 hours. After the reaction was completed, the reaction solution was quenched with water (30 ml), extracted three times with ethyl acetate, and dried over anhydrous sodium sulfate. The solution was concentrated to obtain a crude product, which was purified with flash column chromatography (eluted with a gradient of petroleum ether/ethyl acetate=100:0-0:100) to obtain 0.62 g of yellow solid. MS (m/z): 313.2 [M+H]⁺.

The following intermediates were prepared according to the preparation procedure of intermediate 13-1 using corresponding starting materials and reagents under appropriate conditions that will be recognized by those skilled in the art.

| | | MS |
|---|---|---|
| Intermediate | Structural formula | (M + H)⁺ |
| 13-2 | 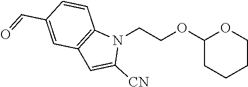 [SMILES: N#Cc1cc2cc(C=O)ccc2n1CCOC1CCCCO1] | 299.2 |

### Intermediates 14 and 15: 5-formyl-4-methyl-1-(2-((tetrahydro-2H-pyran-2-yl)oxy)ethyl)-1H-indole-2-carboxylic acid, 5-formyl-4-methyl-1-(2-((tetrahydro-2H-pyran-2-yl)oxy)ethyl)-1H-indole-2-carboxamide

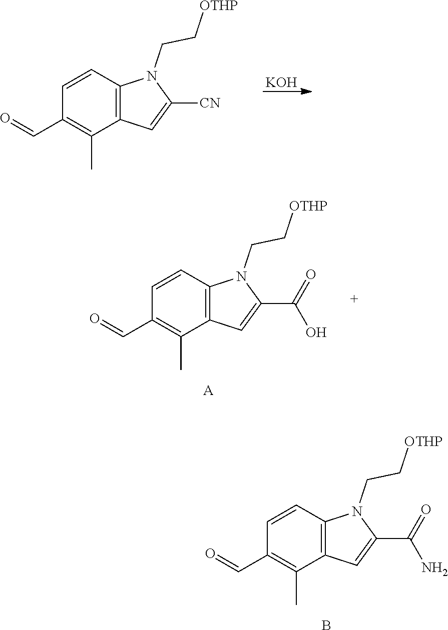

To a reaction flask were added successively 5-formyl-4-methyl-1-(2-((tetrahydro-2H-pyran-2-yl)oxy)ethyl)-1H-indole-2-carbonitrile (intermediate 13-1, 200 mg, 0.64 mmol), potassium hydroxide (143 mg, 2.55 mmol), methanol (8 ml) and water (6 ml), and the mixture was heated to 100° C. and reacted for 16 hours. After the reaction was completed, the reaction solution was concentrated, and the residue was purified with flash column chromatography (eluted with a gradient of water/methanol=100:0-0:100).

Peak 1 is compound A, intermediate 14, carboxylic acid compound 50 mg, MS (m/z): 332.1 [M+H]⁺.

Peak 2 is compound B, intermediate 15, amide compound 150 mg, MS (m/z): 331.1 [M+H]⁺.

### Intermediate 16: tert-butyl 2-cyano-3-(3,6-dihydro-2H-pyran-4-yl)-5-formyl-4-methyl-1H-indole-1-carboxylate

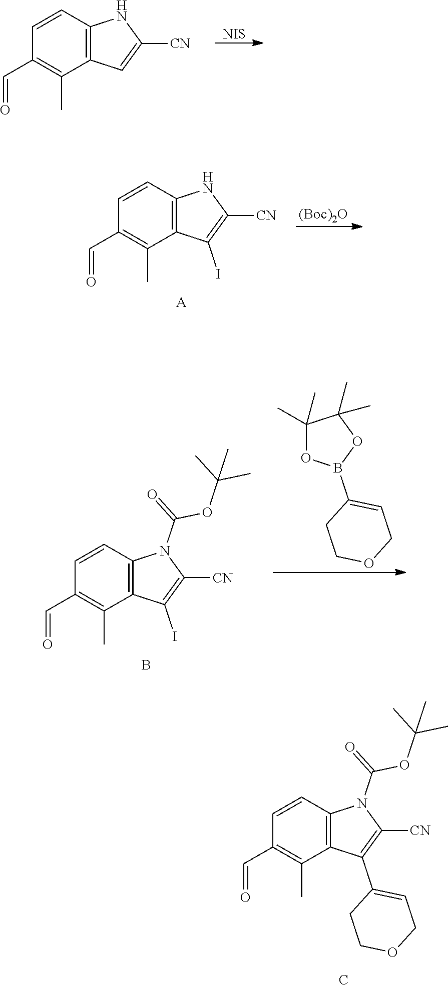

### (A) 5-formyl-3-iodo-4-methyl-1H-indole-2-carbonitrile

To a reaction flask were added 5-formyl-4-methyl-1H-indole-2-carbonitrile (500 mg, 2.71 mmol) and N,N-dimethylformamide (10 ml), followed by N-iodosuccinimide (732 mg, 3.26 mmol), and the mixture was stirred and reacted for 4 hours at room temperature. After the reaction was completed, the reaction solution was poured into water and filtered, and the filter cake was dried to obtain 720 mg of yellow solid. MS (m/z): 311.0 [M+H]⁺.

### (B) tert-butyl 2-cyano-5-formyl-3-iodo-4-methyl-1H-indole-1-carboxylate

To a reaction flask were added 5-formyl-3-iodo-4-methyl-1H-indole-2-carbonitrile (720 mg, 2.32 mmol), di-tert-butyl dicarbonate (1.01 g, 4.63 mmol), 4-dimethylaminopyridine (28 mg, 0.23 mmol) and tetrahydrofuran (50 ml), and the mixture was stirred for 15 hours at room temperature. The reaction solution was concentrated, and the residue was purified with flash column chromatography (eluted with a gradient of water/methanol=100:0-0:100) to obtain 700 mg of white solid. MS (m/z): 433.0 [M+Na]⁺.

### (C) tert-butyl 2-cyano-3-(3,6-dihydro-2H-pyran-4-yl)-5-formyl-4-methyl-1H-indole-1-carboxylate

To a reaction flask were added successively tert-butyl 2-cyano-5-formyl-3-iodo-4-methyl-1H-indole-1-carboxylate (350 mg, 0.85 mmol), 3,6-dihydro-2H-pyran-4-boronic acid pinacol ester (358 mg, 1.70 mmol), Pd(dppf)Cl₂·CH₂Cl₂ (70 mg, 0.086 mmol), potassium carbonate (295 mg, 2.13 mmol), dioxane (20 ml) and water (5 ml), and the mixture was heated to 100° C. and reacted for 3 hours under nitrogen protection. After the reaction was completed, the reaction solution was concentrated, and the residue was purified with flash column chromatography (eluted with a gradient of water/methanol=100:0-0:100) to obtain 200 mg of white solid. MS (m/z): 367.6 [M+H]⁺.

### Intermediate 17: ethyl 3-(2-cyano-5-formyl-4-methyl-1H-indol-1-yl)-2-hydroxypropanoate

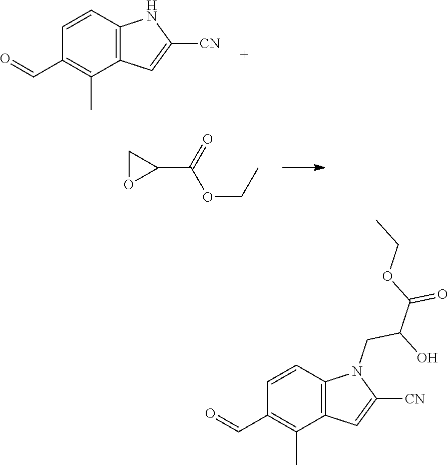

To a reaction flask were added successively 5-formyl-4-methyl-1H-indole-2-carbonitrile (500 mg, 2.71 mmol), ethyl oxiran-2-carboxylate (630 mg, 5.43 mmol), cesium carbonate (1.7 g, 5.22 mmol) and N,N-dimethylformamide (10 ml), and the mixture was heated to 80° C. and reacted for 16 hours. After the reaction was completed, the reaction solution was concentrated, and the residue was purified with flash column chromatography (eluted with a gradient of water/methanol=100:0-0:100) to obtain 100 mg of solid. MS (m/z): 301.1 [M+H]⁺.

### Intermediate 18-1: 5-(4-formylpiperidin-1-yl)picolinonitrile

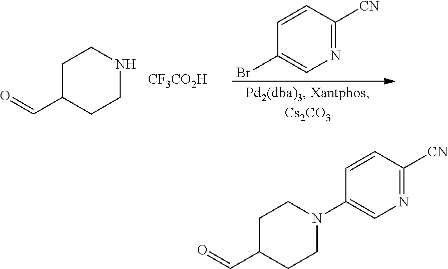 [SMILES: O=CC1CCNCC1 ; "CF3CO2H" ; N#Cc1ccc(Br)cn1 ; N#Cc1ccc(N2CCC(C=O)CC2)cn1]

To a reaction flask were added successively piperidine-4-carbaldehyde (250 mg, 1.1 mmol), 5-bromopicolinonitrile (200 mg, 1.1 mmol), Pd₂(dba)₃ (100 mg, 0.11 mmol), Xantphos (145 mg, 0.25 mmol), cesium carbonate (1 g, 3.1 mmol) and dioxane (10 ml), and the mixture was heated to 100° C. and reacted for 16 hours under nitrogen protection. After the reaction was completed, the reaction solution was concentrated, and the residue was purified with flash column chromatography (eluted with a gradient of water/methanol=100:0-0:100) to obtain 50 mg of oily liquid. MS (m/z): 216.2 [M+H]⁺.

The following intermediates were prepared according to the preparation procedure of intermediate 18-1 using corresponding starting materials and reagents under appropriate conditions that will be recognized by those skilled in the art.

| | | MS |
|---|---|---|
| Intermediate | Structural formula | (M + H)⁺ |
| 18-2 | 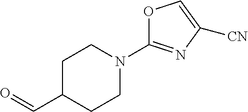 [SMILES: N#Cc1coc(N2CCC(C=O)CC2)n1] | 218.3 |

### Intermediate 19-1: 1-(2-methoxypyridin-4-yl)piperidine-4-carbaldehyde

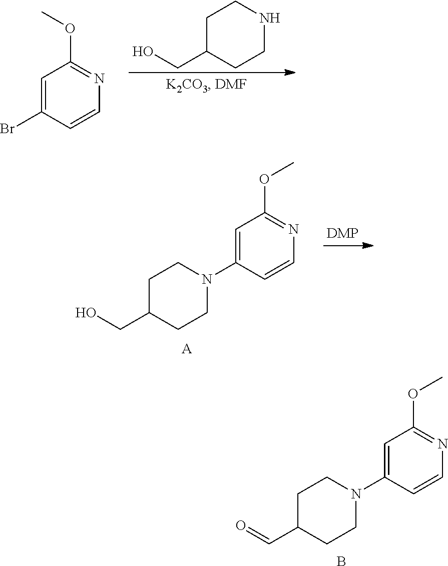

### (A) (1-(2-methoxypyridin-4-yl)piperidin-4-yl)methanol

To a reaction flask were added successively piperidine-4-ylmethanol (615 mg, 5.3 mmol), 4-bromo-2-methoxypyridine (1000 mg, 5.3 mmol), potassium carbonate (2.2 g, 15.9 mmol) and N,N-dimethylformamide (10 ml), and the mixture was heated to 110° C. and reacted for 16 hours. After the reaction was completed, the reaction solution was concentrated, and the residue was purified with flash column chromatography (eluted with a gradient of water/methanol=100:0-0:100) to obtain 500 mg of oily liquid. MS (m/z): 223.2 [M+H]⁺.

### (B) 1-(2-methoxypyridin-4-yl)piperidine-4-carbaldehyde

To a reaction flask were added successively (1-(2-methoxypyridin-4-yl)piperidin-4-yl)methanol (100 mg, 0.45 mmol) and dichloromethane (5 ml), followed by Dess-Martin periodinane (382 mg, 0.9 mmol), and the mixture was reacted at room temperature for 3 hours. After the reaction was completed, the reaction solution was concentrated, and the residue was purified with flash column chromatography (eluted with a gradient of water/methanol=100:0-0:100) to obtain 50 mg of oily liquid. MS (m/z): 221.1 [M+H]⁺.

The following intermediates were prepared according to the preparation procedure of intermediate 19-1 using corresponding starting materials and reagents under appropriate conditions that will be recognized by those skilled in the art.

| | | MS |
|---|---|---|
| Intermediate | Structural formula | (M + H)⁺ |
| 19-2 | 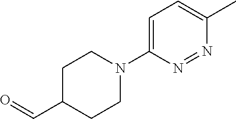 | 206.2 |
| 19-3 | 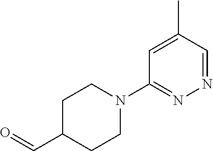 | 206.2 |
| 19-4 | 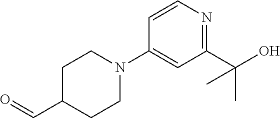 [SMILES: CC(C)(O)c1cc(N2CCC(C=O)CC2)ccn1] | 249.2 |

### Intermediate 20: 2-((4-formyl-3-methylphenyl)amino)acetonitrile

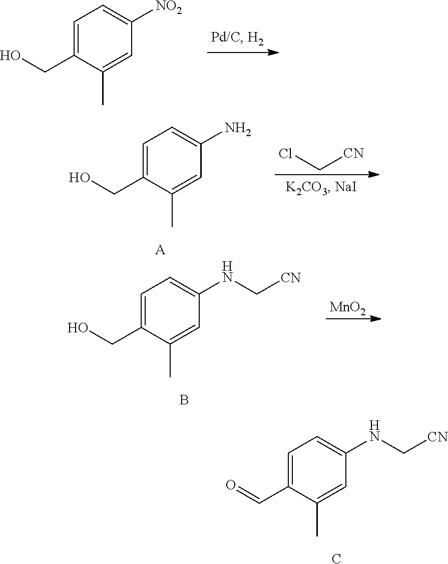

### (A) (4-amino-2-methylphenyl)methanol

To a reaction flask were added (2-methyl-4-nitrophenyl)methanol (500 mg, 3.0 mmol), palladium carbon (100 mg) and ethanol (20 ml), and the mixture was stirred and reacted under hydrogen at room temperature for 15 hours. After the reaction was completed, the reaction solution was filtered, and the filtrate was concentrated to obtain 413 mg of yellow solid. MS (m/z): 138.2 [M+H]⁺.

### (B) 2-((4-(hydroxymethyl)-3-methylphenyl)amino)acetonitrile

To a sealed tube were added (4-amino-2-methylphenyl)methanol (413 mg, 3.0 mmol), 2-chloroacetonitrile (227 mg, 3.0 mmol), potassium carbonate (1.24 g, 9.0 mmol), sodium iodide (450 mg, 3.0 mmol) and acetonitrile (50 ml), and then the mixture was heated to reflux, stirred and reacted for 15 hours. After the reaction was completed, the reaction solution was concentrated, and the residue was purified with flash column chromatography (eluted with a gradient of water/methanol=100:0-0:100) to obtain 200 mg of yellow solid. MS (m/z): 177.2 [M+H]⁺.

### (C) 2-((4-formyl-3-methylphenyl)amino)acetonitrile

To a reaction flask were added 2-((4-(hydroxymethyl)-3-methylphenyl)amino) acetonitrile (150 mg, 0.851 mmol), manganese dioxide (592 mg, 6.81 mmol) and dichloromethane (20 ml), and the mixture was stirred for 15 hours at room temperature. After the reaction was completed, the reaction solution was filtered, the filtrate was concentrated, and then the residue was purified with flash column chromatography (eluted with a gradient of water/methanol=100:0-0:100) to obtain 12 mg of yellow solid. MS (m/z): 175.2 [M+18]⁺.

### Intermediate 21: 1-(6-methoxypyridazin-4-yl)piperidine-4-carbaldehyde

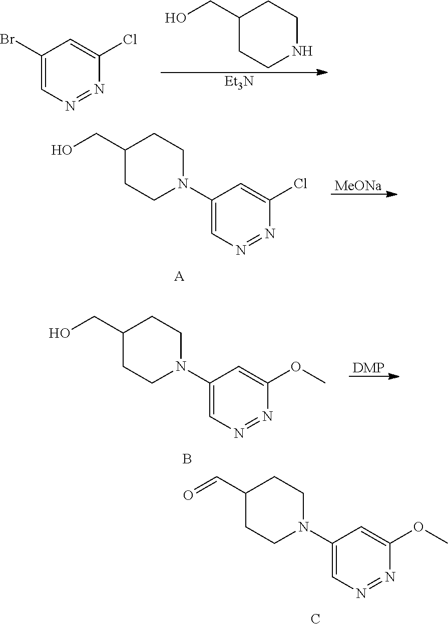

### (A) (1-(6-chloropyridazin-4-yl)piperidin-4-yl)methanol

To a reaction flask were added successively piperidine-4-ylmethanol (576 mg, 5 mmol), 5-bromo-3-chloropyridazine (967 mg, 5 mmol), triethylamine (3 ml) and dioxane (20 ml), and the mixture was heated to 100° C. and reacted for 16 hours. After the reaction was completed, the reaction solution was concentrated, and the residue was purified with flash column chromatography (eluted with a gradient of water/methanol=100:0-0:100) to obtain 620 mg of oily liquid. MS (m/z): 228.1 [M+H]⁺.

### (B) (1-(6-methoxypyridazin-4-yl)piperidin-4-yl)methanol

To a reaction flask were added successively (1-(6-chloropyridazin-4-yl)piperidin-4-yl)methanol (620 mg, 2.72 mmol), sodium methoxide (734 mg, 13.6 mmol) and dioxane (20 ml), and the mixture was heated to 100′T and reacted for 16 hours. After the reaction was completed, the reaction solution was concentrated, and the residue was purified with flash column chromatography (eluted with a gradient of water/methanol=100:0-0:100) to obtain 450 mg of oily liquid. MS (m/z): 224.2 [M+H]⁺.

### (C) 1-(6-methoxypyridazin-4-yl)piperidine-4-carbaldehyde

To a reaction flask were added successively (1-(6-methoxypyridazin-4-yl)piperidin-4-yl)methanol (420 mg, 1.88 mmol) and dichloromethane (25 ml), followed by Dess-Martin periodinane (1595 mg, 3.76 mmol), and the mixture was reacted at room temperature for 3 hours. After the reaction was completed, the reaction solution was concentrated, and the residue was purified with flash column chromatography (eluted with a gradient of water/methanol=100:0-0:100) to obtain 170 mg of oily liquid. MS (m/z): 222.2 [M+H]⁺.

### Intermediate 22: tert-butyl (6-(4-formylpiperidin-1-yl)pyridazin-3-yl)carbamate

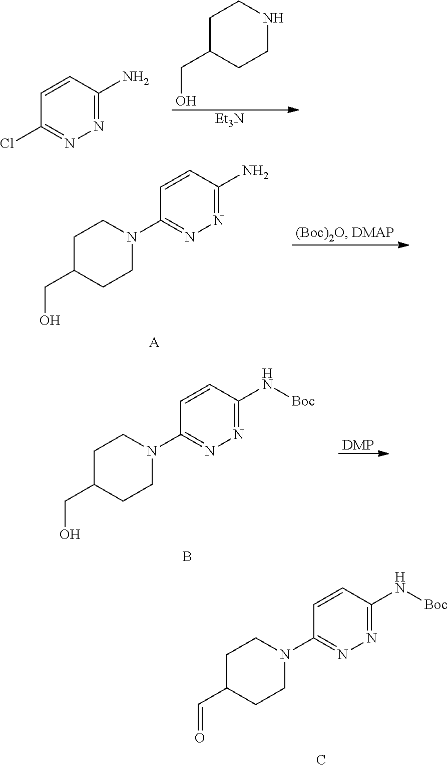

### (A) (1-(6-aminopyridazin-3-yl)piperidin-4-yl)methanol

To a reaction flask were added successively 6-chloropyridazin-3-amine (1.0 g, 7.72 mmol), piperidine-4-ylmethanol (0.98 g, 8.50 mmol), triethylamine (1.54 g, 15.2 mmol) and tert-butanol (30 ml), and the mixture was heated to 200° C. under microwave and reacted for 2 hours. After the reaction was completed, the reaction solution was concentrated, and the residue was purified with flash column chromatography (eluted with a gradient of water/methanol=100:0-0:100) to obtain 0.51 g of light yellow solid. MS (m/z): 209.4 [M+H]⁺.

### (B) tert-butyl (6-(4-(hydroxymethyl)piperidin-1-yl)pyridazin-3-yl)carbamate

To a reaction flask were added successively (1-(6-aminopyridazin-3-yl)piperidin-4-yl)methanol (0.51 g, 2.45 mmol), di-tert-butyl dicarbonate (0.52 g, 2.38 mmol), 4-dimethylaminopyridine (30 mg, 0.245 mmol) and 1,4-dioxane (20 ml), and the mixture was heated to 80° C. and reacted for 2 hours. After the reaction was completed, the reaction solution was concentrated, and the residue was purified with flash column chromatography (eluted with a gradient of water/methanol=100:0-0:100) to obtain 0.18 g of light yellow solid. MS (m/z): 309.2 [M+H]⁺.

### (C) tert-butyl (6-(4-formylpiperidin-1-yl)pyridazin-3-yl)carbamate

To a reaction flask were added tert-butyl (6-(4-(hydroxymethyl)piperidin-1-yl)pyridazin-3-yl)carbamate (180 mg, 0.58 mmol), dichloromethane (10 ml) and Dess-Martin periodinane (492 mg, 1.16 mmol). The mixture was stirred for 2 hours at room temperature. After the reaction was completed, the reaction solution was washed with a saturated sodium bicarbonate solution and a sodium bisulfite solution, the organic phase was concentrated, and the residue was purified with flash column chromatography (eluted with a gradient of water/methanol=100:0-0:100) to obtain 230 mg of product. MS (m/z): 307.2 [M+H]⁺.

### Intermediate 23: methyl 4-(4-formylpiperidin-1-yl)nicotinate

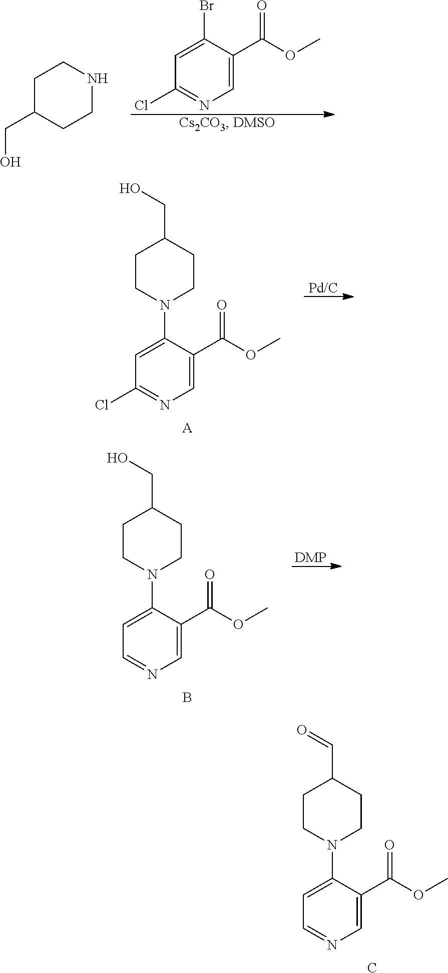

### (A) methyl 6-chloro-4-(4-(hydroxymethyl)piperidin-1-yl)nicotinate

To a reaction flask were added successively methyl 4-bromo-6-chloronicotinate (1.25 g, 5.0 mmol), piperidine-4-ylmethanol (0.57 g, 5.0 mmol), cesium carbonate (3.25 g, 10.0 mmol) and dimethyl sulfoxide (20 ml), and the mixture was heated to 60° C. and reacted for 5 hours. The reaction solution was quenched with water (20 ml), extracted with ethyl acetate, washed with saturated brine, dried over anhydrous sodium sulfate, and concentrated to obtain a crude product, which was purified with flash column chromatography (eluted with a gradient of water/methanol=100:0-0:100) to obtain 0.46 g of light yellow solid. MS (m/z): 285.2 [M+H]⁺.

### (B) methyl 4-(4-(hydroxymethyl)piperidin-1-yl)nicotinate

To a reaction flask were added successively methyl 6-chloro-4-(4-(hydroxymethyl)piperidin-1-yl)nicotinate (0.46 g, 1.62 mmol), palladium carbon (46 mg) and methanol (20 ml), and the mixture was reacted for 2 hours under hydrogen. After the reaction was completed, the reaction solution was filtered, and the filtrate was concentrated to obtain 0.39 g of white solid, which was directly used in the next reaction. MS (m/z): 251.2 [M+H]⁺.

### (C) methyl 4-(4-formylpiperidin-1-yl)nicotinate

To a reaction flask were added methyl 4-(4-(hydroxymethyl)piperidin-1-yl)nicotinate (390 mg, 1.56 mmol) and dichloromethane (20 ml), and Dess-Martin periodinane (992 mg, 2.34 mmol) was added in batches under an ice-bath. The mixture was stirred for 4 hours at room temperature. After the reaction was completed, the reaction solution was washed with a saturated sodium bicarbonate solution and a sodium bisulfite solution, the organic phase was concentrated, and the residue was purified with flash column chromatography (eluted with a gradient of water/acetonitrile=100:0-0:100) to obtain 160 mg of product. MS (m/z): 249.2 [M+H]⁺.

### Intermediate 24-1: 1-(6-methoxypyridazin-3-yl)piperidine-4-carbaldehyde

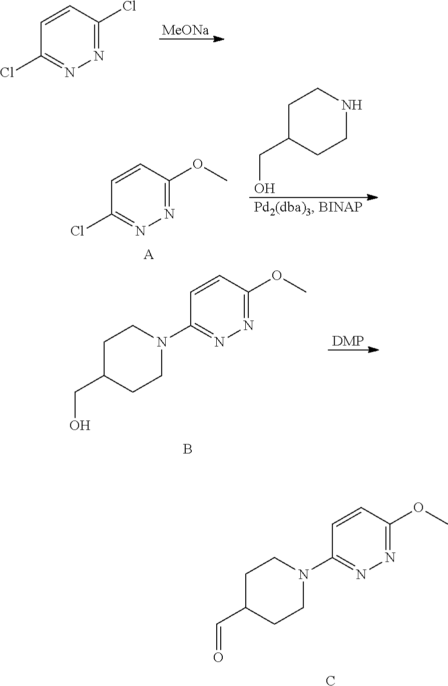

### (A) 3-chloro-6-methoxypyridazine

To a reaction flask were added successively 3,6-dichloropyridazine (1.5 g, 10 mmol), sodium methoxide (2.72 g, 50 mmol) and methanol (30 ml), and the mixture was stirred for 15 hours at room temperature. After the reaction was completed, the reaction solution was concentrated, and the residue was dissolved in dichloromethane, washed with water and saturated sodium chloride, dried over anhydrous sodium sulfate, and concentrated to obtain 1.0 g of white solid. MS (m/z): 145.2, 147.2 [M+H]⁺.

### (B) (1-(6-methoxypyridazin-3-yl)piperidin-4-yl)methanol

To a reaction flask were added successively 3-chloro-6-methoxypyridazine (1.0 g, 6.92 mmol), piperidine-4-ylmethanol (797 mg, 6.92 mmol), Pd₂(dba)₃ (317 mg, 0.35 mmol), BINAP (430 mg, 0.65 mmol), sodium tert-butoxide (1.33 g, 13.84 mmol) and toluene (18 ml), and the mixture was heated to 100° C. and reacted for 15 hours under nitrogen protection. After the reaction was completed, the reaction solution was concentrated, and the residue was purified with flash column chromatography (eluted with a gradient of water/methanol=100:0-0:100) to obtain 300 mg of yellow oil. MS (m/z): 224.2 [M+H]⁺.

### (C) 1-(6-methoxypyridazin-3-yl)piperidine-4-carbaldehyde

To a reaction flask were added (1-(6-methoxypyridazin-3-yl)piperidin-4-yl)methanol (300 mg, 1.34 mmol), dichloromethane (20 ml) and Dess-Martin periodinane (855 mg, 2.02 mmol). The mixture was stirred for 3 hours at room temperature. After the reaction was completed, the reaction solution was washed with a saturated sodium bicarbonate solution and a sodium bisulfite solution, the organic phase was concentrated, and the residue was purified with flash column chromatography (eluted with a gradient of water/methanol=100:0-0:100) to obtain 120 mg of product. MS (m/z): 222.2 [M+H]⁺.

The following intermediates were prepared according to the preparation procedure of intermediate 24-1 using corresponding starting materials and reagents under appropriate conditions that will be recognized by those skilled in the art.

| | | MS |
|---|---|---|
| Intermediate | Structural formula | (M + H)⁺ |
| 24-2 | 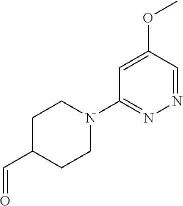 [SMILES: COc1cnnc(N2CCC(C=O)CC2)c1] | 222.2 |
| 24-3 | 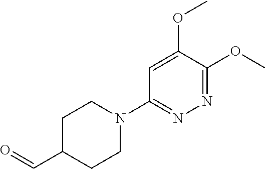 [SMILES: COc1cc(N2CCC(C=O)CC2)nnc1OC] | 252.2 |
| 24-4 | 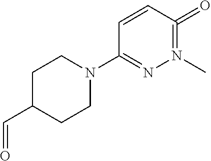 | 221.2 |
| 24-5 | 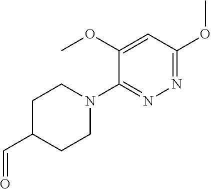 [SMILES: COc1cc(OC)c(N2CCC(C=O)CC2)nn1] | 252.2 |
| 24-6 | 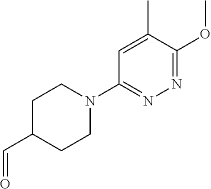 [SMILES: COc1nnc(N2CCC(C=O)CC2)cc1C] | 268.2* |
| 24-7 | 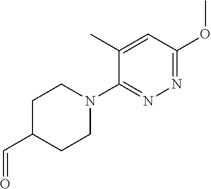 [SMILES: COc1cc(C)c(N2CCC(C=O)CC2)nn1] | 268.2* |
| 24-8 | 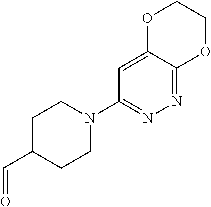 [SMILES: O=CC1CCN(c2cc3c(nn2)OCCO3)CC1] | 250.4 |
| 24-9 | 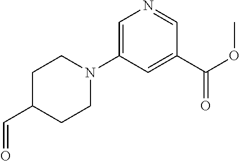 [SMILES: COC(=O)c1cncc(N2CCC(C=O)CC2)c1] | 249.2 |
| 24-10 | 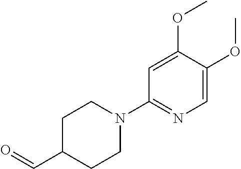 [SMILES: COc1cnc(N2CCC(C=O)CC2)cc1OC] | 251.0 |
| 24-11 | 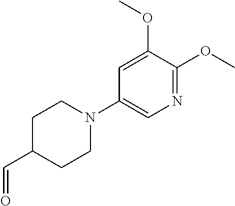 [SMILES: COc1cc(N2CCC(C=O)CC2)cnc1OC] | 251.0 |
| 24-12 | 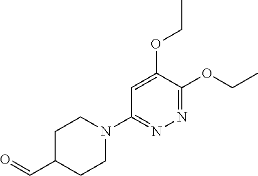 [SMILES: CCOc1cc(N2CCC(C=O)CC2)nnc1OCC] | 280.1 |
| 24-13 | 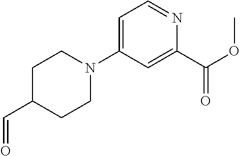 [SMILES: COC(=O)c1cc(N2CCC(C=O)CC2)ccn1] | 249.2 |

*MS shows the data as the molecular weight of acetal

### Intermediate 25: 6-(4-formylpiperidin-1-yl)picolinamide

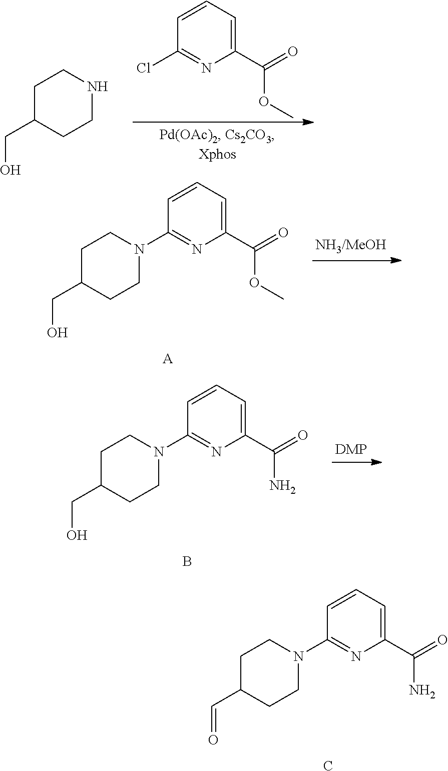

### (A) methyl 6-(4-(hydroxymethyl)piperidin-1-yl)picolinate

To a reaction flask were added successively methyl 6-chloropicolinate (1.71 g, 10 mmol), piperidine-4-ylmethanol (1.15 g, 10 mmol), palladium acetate (224 mg, 1.0 mmol), Xphos (478 mg, 1.0 mmol), cesium carbonate (6.50 g, 20.0 mmol) and 1,4-dioxane (30 ml), and the mixture was heated to 80° C. and reacted for 15 hours under nitrogen protection. After the reaction was completed, the reaction solution was filtered, the filtrate was concentrated, and the residue was purified with flash column chromatography (eluted with a gradient of water/methanol=100:0-0:100) to obtain 1.17 g of yellow oil. MS (m/z): 251.2 [M+H]⁺.

### (B) 6-(4-(hydroxymethyl)piperidin-1-yl)picolinamide

To a sealed tube were added successively methyl 6-(4-(hydroxymethyl)piperidin-1-yl)picolinate (1.17 g, 4.67 mmol) and ammonia in methanol (20 ml, 7 mole/liter), and the mixture was heated to 80° C. and reacted for 2 hours. After the reaction was completed, the reaction solution was concentrated to obtain 0.80 g of light yellow solid, which was directly used in the next reaction. MS (m/z): 236.2 [M+H]⁺.

### (C) 6-(4-formylpiperidin-1-yl)picolinamide

To a reaction flask were added 6-(4-(hydroxymethyl)piperidin-1-yl)picolinamide (235 mg, 1.0 mmol) and dichloromethane (10 ml), and Dess-Martin periodinane (848 mg, 2.0 mmol) was added in batches under an ice-bath. The mixture was stirred for 15 hours at room temperature. After the reaction was completed, the reaction solution was washed with a saturated sodium bicarbonate solution and a sodium bisulfite solution, the organic phase was concentrated, and the residue was purified with flash column chromatography (eluted with a gradient of petroleum ether/ethyl acetate=100:0-0:100) to obtain 57 mg of product. MS (m/z): 234.1 [M+H]⁺.

### Intermediate 26: 1-(4-methoxypyridin-2-yl)piperidine-4-carbaldehyde

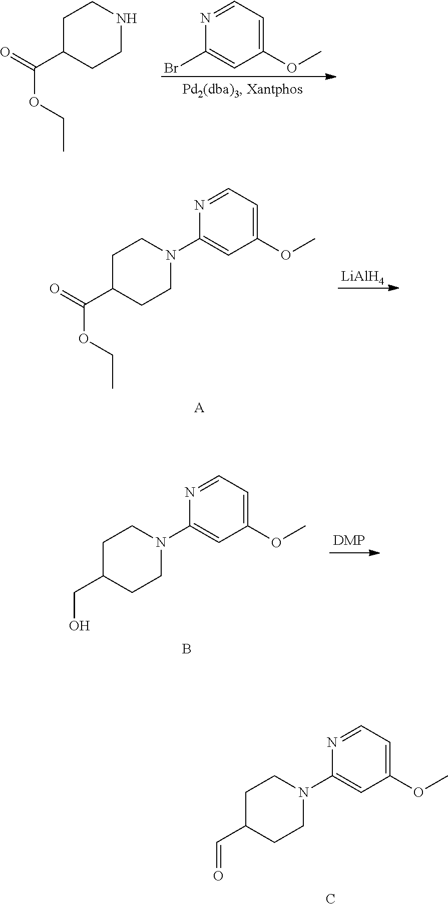

### (A) ethyl 1-(4-methoxypyridin-2-yl)piperidine-4-carboxylate

To a reaction flask were added successively 2-bromo-4-methoxypyridine (500 mg, 2.67 mmol), ethyl piperidine-4-carboxylate (628 mg, 4 mmol), Pd₂(dba)₃ (244 mg, 0.267 mmol), Xantphos (308 mg, 0.534 mmol), cesium carbonate (1.7 g, 5.22 mmol) and dioxane (20 ml), and the mixture was heated to 100° C. and reacted for 16 hours under nitrogen protection. After the reaction was completed, the reaction solution was concentrated, and the residue was purified with flash column chromatography (eluted with a gradient of water/methanol=100:0-0:100) to obtain 100 mg of oily liquid. MS (m/z): 265.2 [M+H]⁺.

### (B) (1-(4-methoxypyridin-2-yl)piperidin-4-yl)methanol

To a reaction flask were added successively ethyl 1-(4-methoxypyridin-2-yl)piperidine-4-carboxylate (100 mg, 0.38 mmol) and tetrahydrofuran (5 ml), and the mixture was cooled to 0° C. Lithium aluminum hydride (29 mg, 0.76 mmol) was added, and the mixture was reacted at room temperature for 5 hours. After the reaction was completed, the reaction solution was concentrated, and the residue was purified with flash column chromatography (eluted with a gradient of water/methanol=100:0-0:100) to obtain 60 mg of oily liquid. MS (m/z): 223.2 [M+H]⁺.

### (C) 1-(4-methoxypyridin-2-yl)piperidine-4-carbaldehyde

To a reaction flask were added successively (1-(4-methoxypyridin-2-yl)piperidin-4-yl)methanol (60 mg, 0.27 mmol) and dichloromethane (5 ml), followed by Dess-Martin periodinane (230 mg, 0.54 mmol), and the mixture was reacted at room temperature for 3 hours. After the reaction was completed, the reaction solution was concentrated, and the residue was purified with flash column chromatography (eluted with a gradient of water/methanol=100:0-0:100) to obtain 40 mg of oily liquid. MS (m/z): 221.2 [M+H]⁺.

### Intermediate 27-1: 1-(6-(((tert-butyldimethylsilyl)oxy)methyl)pyridin-2-yl)piperidine-4-carbaldehyde

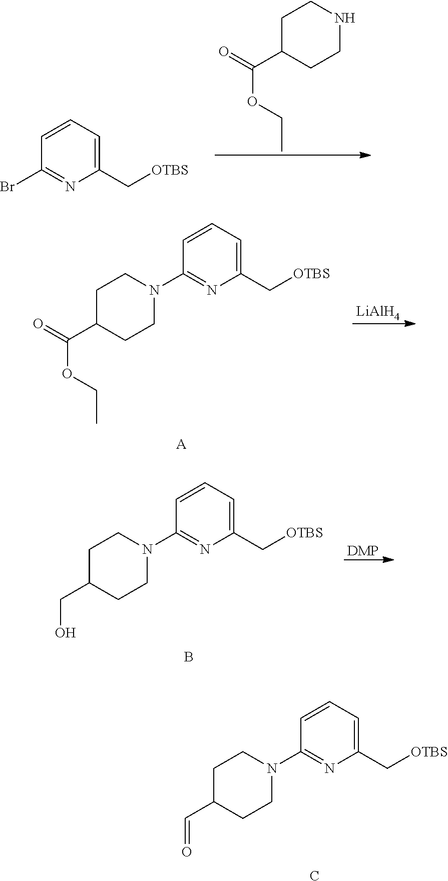

### (A) ethyl 1-(6-(((tert-butyldimethylsilyl)oxy)methyl)pyridin-2-yl)piperidine-4-carboxylate

To a reaction flask were added successively 2-bromo-6-(((tert-butyldimethylsilyl) oxy)methyl)pyridine (500 mg, 1.66 mmol), ethyl piperidine-4-carboxylate (521 mg, 3.32 mmol) and toluene (5 ml), and the mixture was heated to 120° C. and reacted for 16 hours.

After the reaction was completed, the reaction solution was concentrated, and the residue was purified with flash column chromatography (eluted with a gradient of water/methanol=100:0-0:100) to obtain 150 mg of oily liquid. MS (m/z): 379.2 [M+H]⁺.

### (B) (1-(6-(((tert-butyldimethylsilyl)oxy)methyl)pyridin-2-yl)piperidin-4-yl)methanol

To a reaction flask were added successively ethyl 1-(6-(((tert-butyldimethylsilyl) oxy)methyl)pyridin-2-yl)piperidine-4-carboxylate (150 mg, 0.40 mmol) and tetrahydrofuran (5 ml), and the mixture was cooled to 0° C. Lithium aluminum hydride (30 mg, 0.79 mmol) was added, and the mixture was reacted at room temperature for 5 hours. After the reaction was completed, the reaction solution was concentrated, and the residue was purified with flash column chromatography (eluted with a gradient of water/methanol=100:0-0:100) to obtain 100 mg of oily liquid. MS (m/z): 337.2 [M+H]⁺.

### (C) 1-(6-(((tert-butyldimethylsilyl)oxy)methyl)pyridin-2-yl)piperidine-4-carbaldehyde

To a reaction flask were added successively (1-(6-(((tert-butyldimethylsilyl) oxy)methyl)pyridin-2-yl)piperidin-4-yl)methanol (100 mg, 0.3 mmol) and dichloromethane (5 ml), followed by Dess-Martin periodinane (252 mg, 0.59 mmol), and the mixture was reacted at room temperature for 3 hours. After the reaction was completed, the reaction solution was concentrated, and the residue was purified with flash column chromatography (eluted with a gradient of water/methanol=100:0-0:100) to obtain 50 mg of oily liquid. MS (m/z): 335.3 [M+H]⁺.

The following intermediates were prepared according to the preparation procedure of intermediate 27-1 using corresponding starting materials and reagents under appropriate conditions that will be recognized by those skilled in the art.

| | | MS |
|---|---|---|
| Intermediate | Structural formula | (M + H)⁺ |
| 27-2 | 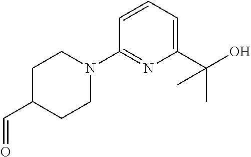 [SMILES: CC(C)(O)c1cccc(N2CCC(C=O)CC2)n1] | 249.2 |

### Intermediate 28: 4-(pyridin-2-yl)benzaldehyde

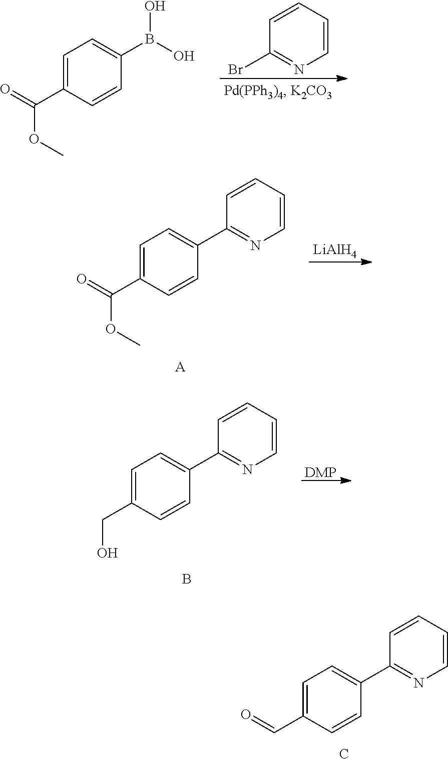

### (A) methyl 4-(pyridin-2-yl)benzoate

To a reaction flask were added successively (4-(methoxycarbonyl)phenyl)boronic acid (800 mg, 4.4 mmol), 2-bromopyridine (500 mg, 3.2 mmol), tetra(triphenylphosphine)palladium (367 mg, 0.32 mmol), potassium carbonate (1 g, 7.2 mmol), dioxane (8 ml) and water (2 ml), and the mixture was heated to 100° C. and reacted for 5 hours under nitrogen protection. After the reaction was completed, the reaction solution was concentrated, and the residue was purified with flash column chromatography (eluted with a gradient of PE/EA=100:0-0:100) to obtain 500 mg of oily liquid. MS (m/z): 214.1 [M+H]⁺.

### (B) (4-(pyridin-2-yl)phenyl)methanol

To a reaction flask were added successively methyl 4-(pyridin-2-yl)benzoate (200 mg, 0.94 mmol) and tetrahydrofuran (10 ml), and the mixture was cooled to 0° C. Lithium aluminum hydride (71 mg, 1.88 mmol) was added, and the mixture was reacted at room temperature for 16 hours. After the reaction was completed, the reaction solution was concentrated, and the residue was purified with flash column chromatography (eluted with a gradient of water/methanol=100:0-0:100) to obtain 150 mg of oily liquid. MS (m/z): 186.1 [M+H]⁺.

### (C) 4-(pyridin-2-yl)benzaldehyde

To a reaction flask were added successively (4-(pyridin-2-yl)phenyl)methanol (150 mg, 0.81 mmol) and dichloromethane (5 ml), followed by Dess-Martin periodinane (688 mg, 1.62 mmol), and the mixture was reacted at room temperature for 3 hours. After the reaction was completed, the reaction solution was concentrated, and the residue was purified with flash column chromatography (eluted with a gradient of water/methanol=100:0-0:100) to obtain 100 mg of oily liquid. MS (m/z): 184.1 [M+H]⁺.

### Intermediate 29: 4-(pyridin-2-yl)cyclohex-3-ene-1-carbaldehyde

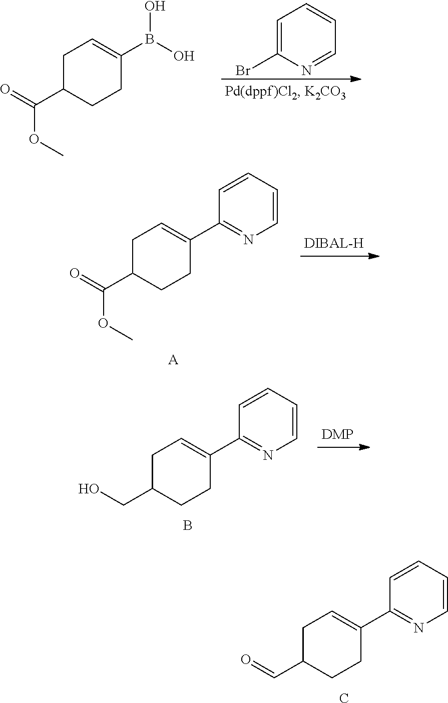

### (A) methyl 4-(pyridin-2-yl)cyclohex-3-ene-1-carboxylate

To a reaction flask were added successively (4-(methyloxycarbonyl)cyclohex-1-ene-1-yl)boronic acid (873 mg, 4.75 mmol), 2-bromopyridine (500 mg, 3.16 mmol), Pd(dppf)Cl₂·CH₂Cl₂ (130 mg, 0.18 mmol), potassium carbonate (1.1 g, 8.0 mmol), dioxane (30 ml) and water (6 ml), and the mixture was heated to 100° C. and reacted for 5 hours under nitrogen protection. After the reaction was completed, the reaction solution was concentrated, and the residue was purified with flash column chromatography (eluted with a gradient of water/methanol=100:0-0:100) to obtain 500 mg of yellow solid. MS (m/z): 218.2 [M+H]⁺.

### (B) (4-(pyridin-2-yl)cyclohex-3-en-1-yl)methanol

To a reaction flask were added methyl 4-(pyridin-2-yl)cyclohex-3-ene-1-carboxylate (500 mg, 2.30 mmol) and anhydrous tetrahydrofuran (15 ml), and the mixture was cooled to 0° C. Diisobutylaluminium hydride (2.76 ml, 2.76 mmol) was added dropwise under nitrogen protection, and then the mixture was warmed to room temperature and stirred for 15 hours. The reaction solution was quenched with a saturated ammonium chloride solution and concentrated, and the residue was purified with flash column chromatography (eluted with a gradient of water/methanol=100:0-0:100) to obtain 400 mg of yellow oil. MS (m/z): 190.2 [M+H]⁺.

### (C) 4-(pyridin-2-yl)cyclohex-3-ene-1-carbaldehyde

To a reaction flask were added (4-(pyridin-2-yl)cyclohex-3-en-1-yl)methanol (400 mg, 2.11 mmol), dichloromethane (20 ml) and Dess-Martin periodinane (1.8 g, 4.24 mmol). The mixture was stirred for 3 hours at room temperature. After the reaction was completed, the reaction solution was washed with a saturated sodium bicarbonate solution and a sodium bisulfite solution, the organic phase was concentrated, and the residue was purified with flash column chromatography (eluted with a gradient of petroleum ether/ethyl acetate=100:0-0:100) to obtain 380 mg of white solid. MS (m/z): 188.7 [M+H]⁺.

### Intermediate 30: 2-methoxyquinazoline-6-carbaldehyde

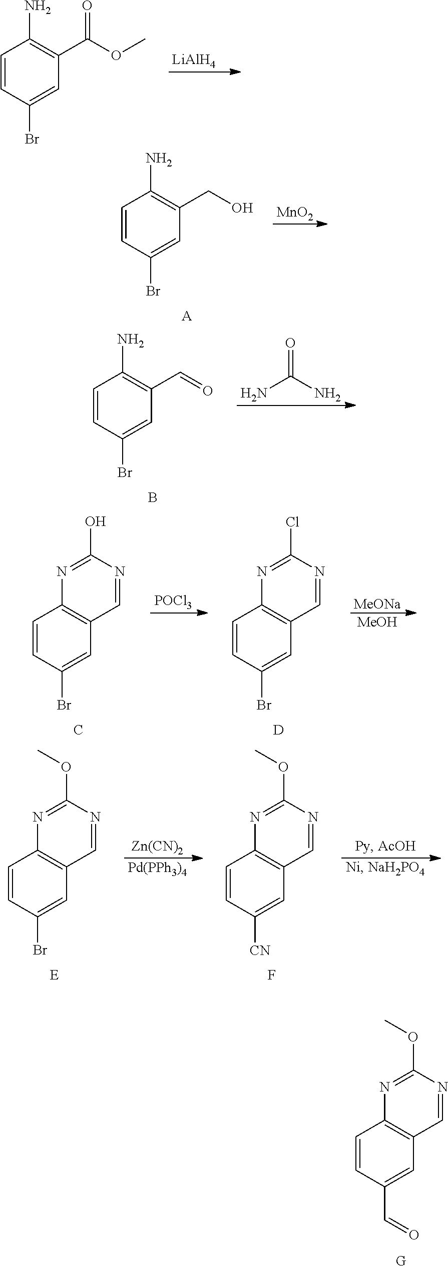

### (A) (2-amino-5-bromophenyl)methanol

To a reaction flask were added lithium aluminum hydride (2.77 g, 73.01 mmol) and anhydrous tetrahydrofuran (40 ml), then methyl 2-amino-5-bromobenzoate (5.6 g, 24.34 mmol) dissolved in tetrahydrofuran (40 ml) was added dropwise, and the mixture was stirred and reacted for 15 hours at room temperature. After the reaction was completed, the reaction solution was quenched with a saturated magnesium sulfate solution (5 ml), and a magnesium sulfate solid (5 g) was added. The mixture was filtered, the filtrate was concentrated, and the residue was purified with flash column chromatography (eluted with a gradient of dichloromethane/methanol=100:0-0:100) to obtain 3.2 g of white solid. MS (m/z): 202.0, 204.0 [M+H]⁺.

### (B) 2-amino-5-bromobenzaldehyde

To a reaction flask were added (2-amino-5-bromophenyl)methanol (3.2 g, 15.84 mmol), manganese dioxide (9.65 g, 111.0 mmol) and dichloromethane (60 ml), and the mixture was stirred for 15 hours at room temperature and filtered. The filtrate was then concentrated, and the residue was purified with flash column chromatography (eluted with a gradient of petroleum ether/ethyl acetate=100:0-0:100) to obtain 2.5 g of yellow solid. MS (m/z): 200.0, 202.0 [M+H]⁺.

### (C) 6-bromoquinazolin-2-ol

To a reaction flask were added 2-amino-5-bromobenzaldehyde (800 mg, 4.0 mmol) and urea (1.92 g, 32.0 mmol), and the mixture was heated to 170° C., stirred and reacted for 1 hour. The reaction solution was cooled to room temperature, and water (30 ml) was added. The mixture was stirred for additional 20 minutes and filtered, and the filter cake was dried to obtain 850 mg of white solid. MS (m/z): 225.0, 227.0 [M+H]⁺.

### (D) 6-bromo-2-chloroquinazoline

To a reaction flask were added 6-bromoquinazolin-2-ol (850 mg, 3.78 mmol) and phosphorus oxychloride (10 ml), and the mixture was heated to reflux overnight. The reaction solution was cooled to room temperature and concentrated. The residue was stirred for 30 minutes in a saturated sodium bicarbonate solution and filtered, and the filter cake was dried to obtain 450 mg of yellow solid. MS (m/z): 243.0, 245.0 [M+H]⁺.

### (E) 6-bromo-2-methoxyquinazoline

To a reaction flask were added 6-bromo-2-chloroquinazoline (450 mg, 1.85 mmol), sodium methoxide (300 mg, 5.54 mmol) and methanol (50 ml), and the mixture was heated to reflux and stirred for 15 hours. The reaction solution was concentrated, and the residue was purified with flash column chromatography (eluted with a gradient of water/methanol=100:0-0:100) to obtain 400 mg of white solid. MS (m/z): 239.0, 241.0 [M+H]⁺.

### (F) 2-methoxyquinazoline-6-carbonitrile

To a reaction flask were added 6-bromo-2-methoxyquinazoline (400 mg, 1.67 mmol), zinc cyanide (235 mg, 2.00 mmol), tetra(triphenylphosphine)palladium (193 mg, 0.167 mmol) and N,N-dimethylformamide (8.0 ml), and the mixture was heated to 100° C. and stirred for 4 hours under nitrogen protection, cooled to room temperature, and purified with flash column chromatography (eluted with a gradient of water/methanol=100:0-0:100) to obtain 200 mg of yellow solid. MS (m/z): 186.1 [M+H]⁺.

### (G) 2-methoxyquinazoline-6-carbaldehyde

To a reaction flask were added 2-methoxyquinazoline-6-carbonitrile (180 mg, 0.97 mmol), acetic acid (5.0 ml) and pyridine (5.0 ml), and then Raney nickel (90 mg) and sodium dihydrogen phosphate monohydrate (401 mg, 2.91 mmol) were added successively. The mixture was heated to 75° C. and stirred for 15 hours, cooled to room temperature and concentrated, and the residue was purified with flash column chromatography (eluted with a gradient of water/methanol=100:0-0:100) to obtain 70 mg of yellow solid. MS (m/z): 189.2 [M+H]⁺.

### Intermediate 31: 2-methylquinazoline-6-carbaldehyde

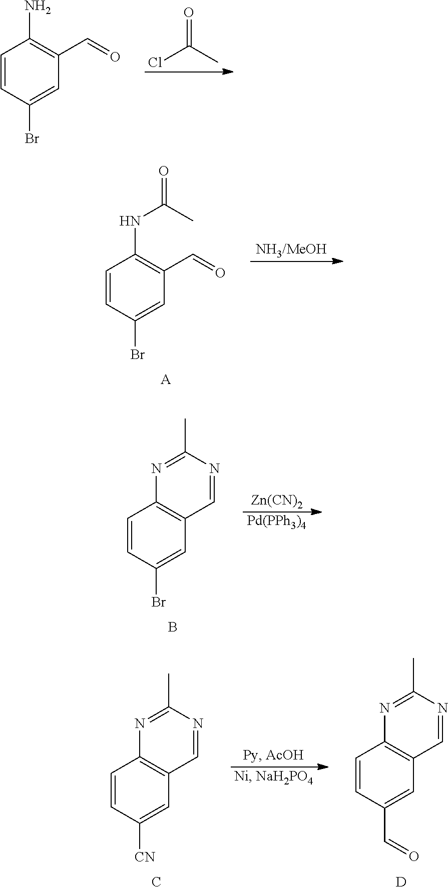

### (A) N-(4-bromo-2-formylphenyl)acetamide

To a reaction flask were added 2-amino-5-bromobenzaldehyde (500 mg, 2.5 mmol), pyridine (395 mg, 5.0 mmol) and dichloromethane (20 ml), then acetyl chloride (235 mg, 3.0 mmol) was added dropwise, and the mixture was stirred and reacted for 15 hours at room temperature. After the reaction was completed, the reaction solution was concentrated, and the residue was purified with flash column chromatography (eluted with a gradient of petroleum ether/ethyl acetate=100:0-0:100) to obtain 420 mg of yellow solid. MS (m/z): 242.0, 244.0 [M+H]⁺.

### (B) 6-bromo-2-methylquinazoline

To a sealed tube were added N-(4-bromo-2-formylphenyl)acetamide (420 mg, 1.74 mmol) and ammonia in methanol (15 ml, 7 mole/liter), and the mixture was heated to 80° C., stirred and reacted for 15 hours under sealed conditions. After the reaction was completed, the reaction solution was concentrated, and the residue was purified with flash column chromatography (eluted with a gradient of petroleum ether/ethyl acetate=100:0-0:100) to obtain 250 mg of yellow solid. MS (m/z): 223.0, 225.0 [M+H]⁺.

### (C) 2-methylquinazoline-6-carbonitrile

To a reaction flask were added 6-bromo-2-methylquinazoline (200 mg, 0.897 mmol), zinc cyanide (105 mg, 0.897 mmol), tetra(triphenylphosphine)palladium (104 mg, 0.09 mmol) and N,N-dimethylformamide (5.0 ml), and the mixture was heated to 100° C. and stirred for 1 hours under nitrogen protection, cooled to room temperature, and purified with flash column chromatography (eluted with a gradient of water/methanol=100:0-0:100) to obtain 100 mg of yellow solid. MS (m/z): 188.2 [M+18]⁺.

### (D) 2-methylquinazoline-6-carbaldehyde

To a reaction flask were added 2-methylquinazoline-6-carbonitrile (100 mg, 0.59 mmol), acetic acid (1.5 ml) and pyridine (1.5 ml), and then Raney nickel (50 mg) and sodium dihydrogen phosphate monohydrate (245 mg, 1.78 mmol) were added successively. The mixture was heated to 75° C. and stirred for 15 hours, cooled to room temperature and concentrated, and the residue was purified with flash column chromatography (eluted with a gradient of water/methanol=100:0-0:100) to obtain 15 mg of yellow solid. MS (m/z): 191.2 [M+18]⁺.

### Intermediate 32: 1-(1-methyl-1H-pyrazol-3-yl)piperidine-4-carbaldehyde

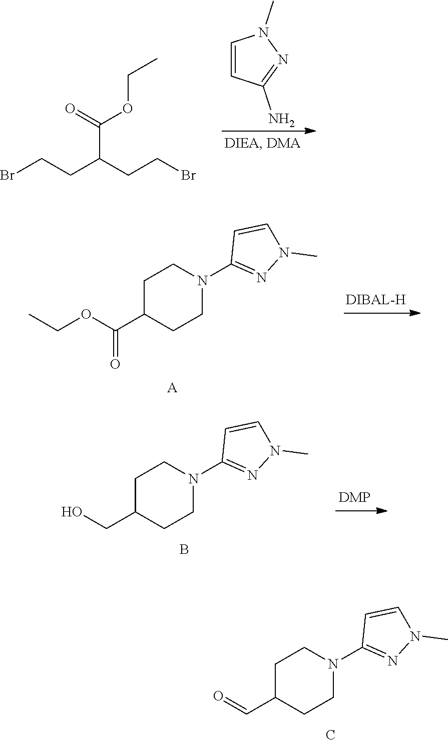

### (A) ethyl 1-(1-methyl-1H-pyrazol-3-yl)piperidine-4-carboxylate

To a reaction flask were added ethyl 4-bromo-2-(2-bromoethyl)butyrate (1.86 g, 6.16 mmol), 1-methyl-1H-pyrazol-3-amine (600 mg, 6.18 mmol), diisopropylethylamine (2.4 g, 18.57 mmol) and N,N-dimethylacetamide (10 ml), and the mixture was heated to 120° C. under microwave and reacted for 2 hours, cooled to room temperature, and purified with flash column chromatography (eluted with a gradient of water/methanol=100:0-0:100) to obtain 1.2 g of white solid. MS (m/z): 238.4 [M+H]⁺.

### (B) (1-(1-methyl-1H-pyrazol-3-yl)piperidin-4-yl)methanol

To a reaction flask were added ethyl 1-(1-methyl-1H-pyrazol-3-yl)piperidine-4-carboxylate (1.2 g, 5.06 mmol) and anhydrous tetrahydrofuran (15 ml), and the mixture was cooled to 0° C. Diisobutylaluminium hydride (6.07 ml, 6.07 mmol, 1.0 mole/liter in toluene) was added dropwise under nitrogen protection, and then the mixture was warmed to room temperature and stirred for 15 hours. The reaction solution was quenched with a saturated ammonium chloride solution and concentrated, and the residue was purified with flash column chromatography (eluted with a gradient of water/methanol=100:0-0:100) to obtain 600 mg of oil. MS (m/z): 196.2 [M+H]⁺.

### (C) 1-(1-methyl-1H-pyrazol-3-yl)piperidine-4-carbaldehyde

To a reaction flask were added (1-(1-methyl-1H-pyrazol-3-yl)piperidin-4-yl)methanol (600 mg, 3.07 mmol), dichloromethane (20 ml) and Dess-Martin periodinane (1.95 g, 4.60 mmol). The mixture was stirred for 2 hours at room temperature. After the reaction was completed, the reaction solution was washed with a saturated sodium bicarbonate solution and a sodium bisulfite solution, the organic phase was concentrated, and the residue was purified with flash column chromatography (eluted with a gradient of water/methanol=100:0-0:100) to obtain 120 mg of yellow solid. MS (m/z): 194.2 [M+H]⁺.

### Intermediate 33: (S)-3-(6-methoxypyridin-3-yl)-2,3-dihydro-[1,4]dioxino[2,3-b]pyridine-7-carbaldehyde

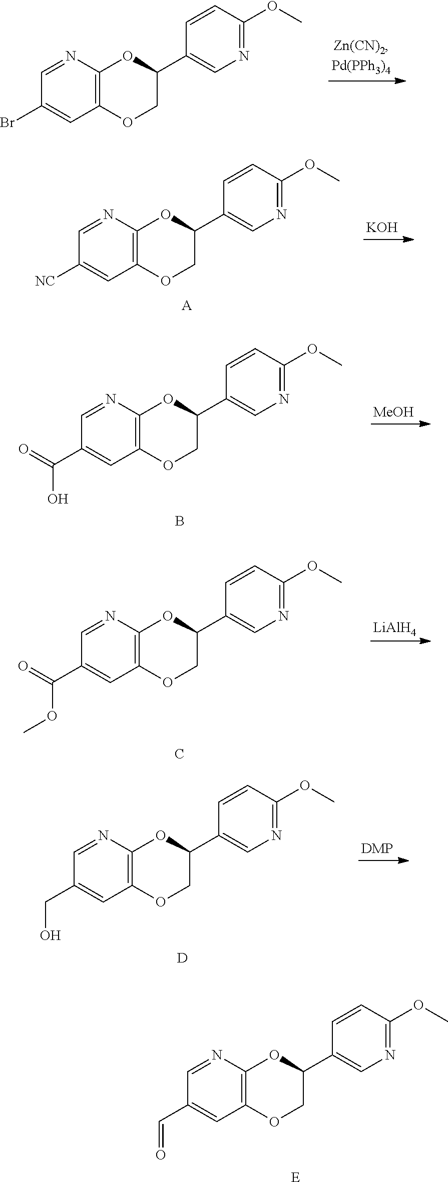

### (A) (S)-3-(6-methoxypyridin-3-yl)-2,3-dihydro-[1,4]dioxino[2,3-b]pyridine-7-carbonitrile

To a reaction flask were added successively (S)-7-bromo-3-(6-methoxypyridin-3-yl)-2,3-dihydro-[1,4]dioxino[2,3-b]pyridine (500 mg, 1.55 mmol), zinc cyanide (145 mg, 1.24 mmol), tetra(triphenylphosphine)palladium (179 mg, 0.155 mmol) and N,N-dimethylformamide (8 ml), and the mixture was heated to 120° C. and reacted for 16 hours under nitrogen protection. After the reaction was completed, the reaction solution was concentrated, and the residue was purified with flash column chromatography (eluted with a gradient of water/methanol=100:0-0:100) to obtain 200 mg of yellow solid. MS (m/z): 270.1 [M+H]⁺.

### (B) (S)-3-(6-methoxypyridin-3-yl)-2,3-dihydro-[1,4]dioxino[2,3-b]pyridine-7-carboxylic acid

To a reaction flask were added successively (S)-3-(6-methoxypyridin-3-yl)-2,3-dihydro-[1,4]dioxino[2,3-b]pyridine-7-carbonitrile (200 mg, 0.74 mmol), potassium hydroxide (166 mg, 2.96 mmol), methanol (5 ml) and water (5 ml), and the mixture was heated to 100° C. and reacted for 16 hours. After the reaction was completed, the reaction solution was concentrated, and the residue was purified with flash column chromatography (eluted with a gradient of water/methanol=100:0-0:100) to obtain 150 mg of white solid. MS (m/z): 289.1 [M+H]⁺.

### (C) methyl (S)-3-(6-methoxypyridin-3-yl)-2,3-dihydro-[1,4]dioxino[2,3-b]pyridine-7-carboxylate

In a reaction flask, (S)-3-(6-methoxypyridin-3-yl)-2,3-dihydro-[1,4]dioxino[2,3-b]pyridine-7-carboxylic acid (150 mg, 0.52 mmol) was dissolved in dichloromethane (5 ml), oxalyl chloride (200 mg, 1.58 mmol) and one drop of N,N-dimethylformamide were added, and the mixture was reacted at room temperature for 2 hours. Methanol (10 ml) was added, and the mixture was reacted at room temperature for 1 hour. After the reaction was completed, the reaction solution was concentrated, and the residue was purified with flash column chromatography (eluted with a gradient of petroleum ether/ethyl acetate=100:0-0:100) to obtain 150 mg of oily liquid. MS (m/z): 303.1 [M+H]⁺.

### (D) (S)-(3-(6-methoxypyridin-3-yl)-2,3-dihydro-[1,4]dioxino[2,3-b]pyridin-7-yl)methanol

To a reaction flask were added successively methyl (S)-3-(6-methoxypyridin-3-yl)-2,3-dihydro-[1,4]dioxino[2,3-b]pyridine-7-carboxylate (150 mg, 0.5 mmol) and tetrahydrofuran (5 ml), and the mixture was cooled to 0° C. Lithium aluminum hydride (75 mg, 1.98 mmol) was added, and the mixture was reacted at room temperature for 3 hours. After the reaction was completed, the reaction solution was concentrated, and the residue was purified with flash column chromatography (eluted with a gradient of water/methanol=100:0-0:100) to obtain 100 mg of oily liquid. MS (m/z): 275.1 [M+H]⁺.

### (E) (S)-3-(6-methoxypyridin-3-yl)-2,3-dihydro-[1,4]dioxino[2,3-b]pyridine-7-carbaldehyde

To a reaction flask were added successively (S)-(3-(6-methoxypyridin-3-yl)-2,3-dihydro-[1,4]dioxino[2,3-b]pyridin-7-yl)methanol (100 mg, 0.36 mmol) and dichloromethane (5 ml), followed by Dess-Martin periodinane (309 mg, 0.73 mmol), and the mixture was reacted at room temperature for 3 hours. After the reaction was completed, the reaction solution was concentrated, and the residue was purified with flash column chromatography (eluted with a gradient of water/methanol=100:0-0:100) to obtain 60 mg of oily liquid. MS (m/z): 273.1 [M+H]⁺.

### Intermediate 34: 5-chloro-6-methoxy-4-methylnicotinaldehyde

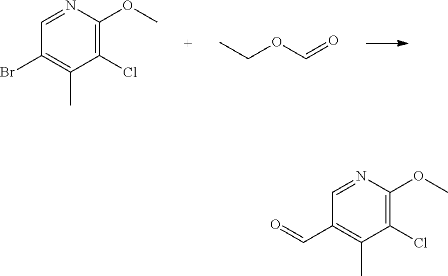

To a reaction flask were added 5-bromo-3-chloro-2-methoxy-4-methylpyridine (200 mg, 0.85 mmol) and anhydrous tetrahydrofuran (8 ml), and the mixture was cooled to −78° C. n-Butyl lithium (0.34 ml, 0.85 mmol) was added dropwise, and after 15 minutes, ethyl formate (63 mg, 0.85 mmol) was added. The mixture was then stirred at room temperature for 2 hours. After the reaction was completed, the reaction solution was concentrated, and the residue was purified with flash column chromatography (eluted with a gradient of water/methanol=100:0-0:100) to obtain 50 mg of oily liquid. MS (m/z): 186.1 [M+H]⁺.

### Intermediate 35: 4-hydroxy-4-(pyridin-2-yl)cyclohexane-1-carbaldehyde

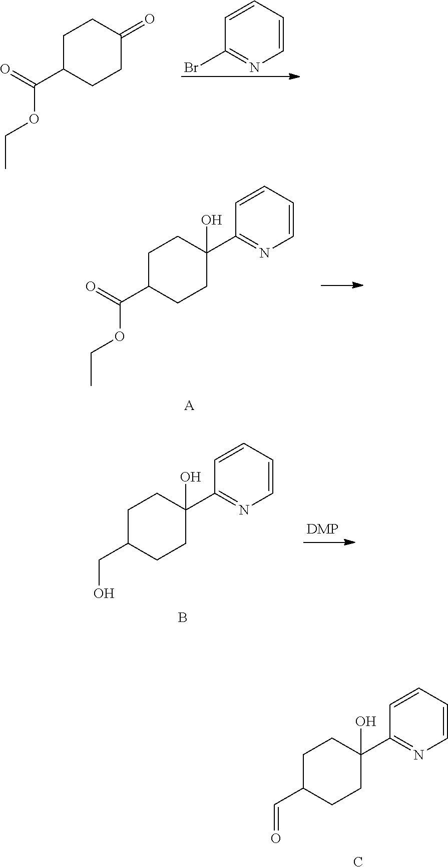

### (A) ethyl 4-hydroxy-4-(pyridin-2-yl)cyclohexane-1-carboxylate

To a reaction flask were added successively 2-bromopyridine (0.87 g, 5.5 mmol) and anhydrous tetrahydrofuran (10 ml), and the mixture was cooled to −78° C. 1.6 mole/liter n-butyl lithium solution (3.4 ml, 5.44 mmol) was added dropwise, and after 30 minutes, ethyl 4-oxocyclohexane-1-carboxylate (0.85 g, 5 mmol) was added dropwise. The mixed solution was reacted at −78° C. for additional 1 hour. The reaction solution was quenched with a saturated aqueous ammonium chloride solution (10 ml) and extracted three times with ethyl acetate. The organic phase was dried and concentrated, and the residue was purified with flash column chromatography (eluted with a gradient of water/methanol=100:0-0:100) to obtain 0.43 g of yellow oil. MS (m/z): 250.2 [M+H]⁺.

### (B) 4-(hydroxymethyl)-1-(pyridin-2-yl)cyclohexan-1-ol

To a reaction flask were added successively ethyl 4-hydroxy-4-(pyridin-2-yl)cyclohexane-1-carboxylate (0.43 g, 1.72 mmol) and tetrahydrofuran (10 ml), and the mixture was cooled under an ice-bath. 1 mole/liter diisobutylaluminium hydride solution (3.44 ml, 3.44 mmol) was added dropwise, and then the mixture was warmed to room temperature and reacted for 2 hours. The reaction solution was quenched with a saturated aqueous ammonium chloride solution (10 ml) and extracted three times with ethyl acetate. The organic phase was dried and concentrated, and the residue was purified with flash column chromatography (eluted with a gradient of water/methanol=100:0-0:100) to obtain 0.24 g of yellow oil. MS (m/z): 208.1 [M+H]⁺.

### (C) 4-hydroxy-4-(pyridin-2-yl)cyclohexane-1-carbaldehyde

To a reaction flask were added 4-(hydroxymethyl)-1-(pyridin-2-yl)cyclohexan-1-ol (0.24 g, 1.16 mmol) and dichloromethane (15 ml), and Dess-Martin periodinane (0.98 g, 2.31 mmol) was added in batches under an ice-bath. The mixture was stirred for 2 hours at room temperature. After the reaction was completed, the reaction solution was washed with a saturated sodium bicarbonate solution and a sodium bisulfite solution, the organic phase was concentrated, and the residue was purified with flash column chromatography (eluted with a gradient of petroleum ether/ethyl acetate=100:0-0:100) to obtain 56 mg of product. MS (m/z): 206.1 [M+H]⁺.

### Intermediate 36: 3-formyl-1-methyl-1H-pyrazole-5-carboxamide

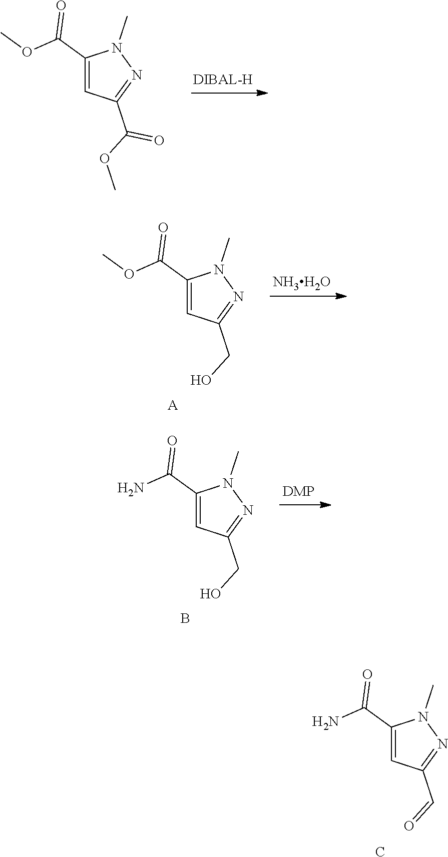

### (A) methyl 3-(hydroxymethyl)-1-methyl-1H-pyrazole-5-carboxylate

To a reaction flask were added successively methyl 1-methyl-1H-pyrazole-3,5-dicarboxylate (991 mg, 5.0 mmol) and tetrahydrofuran (20 ml), and the mixture was cooled to 0° C. 1 mole/liter diisobutylaluminium hydride (10 ml, 10.0 mmol) was added, and the mixture was reacted at room temperature for 2 hours. After the reaction was completed, the reaction solution was concentrated, and the residue was purified with flash column chromatography (eluted with a gradient of water/methanol=100:0-0:100) to obtain 500 mg of oily liquid. MS (m/z): 171.1 [M+H]⁺.

### (B) 3-(hydroxymethyl)-1-methyl-1H-pyrazole-5-carboxamide

To a reaction flask were added successively methyl 3-(hydroxymethyl)-1-methyl-1H-pyrazole-5-carboxylate (500 mg, 2.94 mmol) and aqueous ammonia (10 ml), and the mixture was reacted at room temperature for 15 hours. After the reaction was completed, the reaction solution was concentrated, and the residue was purified with flash column chromatography (eluted with a gradient of water/methanol=100:0-0:100) to obtain 456 mg of oily liquid. MS (m/z): 156.2 [M+H]⁺.

### (C) 3-formyl-1-methyl-1H-pyrazole-5-carboxamide

To a reaction flask were added successively 3-(hydroxymethyl)-1-methyl-1H-pyrazole-5-carboxamide (450 mg, 2.9 mmol) and dichloromethane (25 ml), followed by Dess-Martin periodinane (1230 mg, 2.9 mmol), and the mixture was reacted at room temperature for 15 hours. After the reaction was completed, the reaction solution was concentrated, and the residue was purified with flash column chromatography (eluted with a gradient of water/methanol=100:0-0:100) to obtain 260 mg of oily liquid. MS (m/z): 154.0 [M+H]⁺.

### Intermediate 37: 4-(2-oxopyridin-1(2H)-yl)cyclohexane-1-carbaldehyde

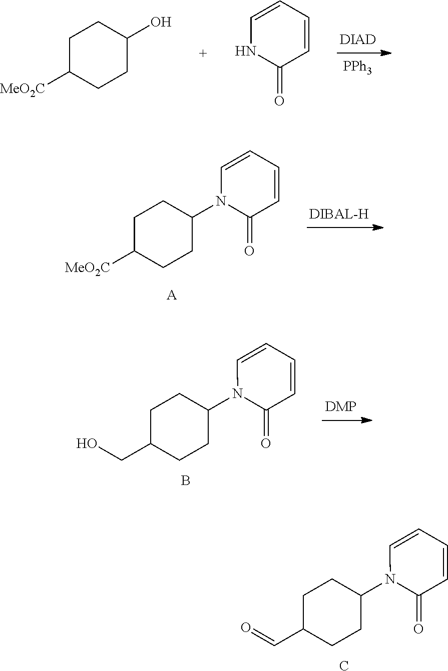

### (A) methyl 4-(2-oxopyridin-1(2H)-yl)cyclohexane-1-carboxylate

To a reaction flask were added successively methyl 4-hydroxycyclohexane-1-carboxylate (1.58 g, 10 mmol), pyridine-2(1H)-one (951 mg, 10 mmol), diisopropyl azodicarboxylate (2.02 g, 10 mmol), triphenylphosphine (2.6 g, 10 mmol) and tetrahydrofuran (40 ml), and the mixture was reacted at room temperature for 16 hours.

After the reaction was completed, the reaction solution was concentrated, and the residue was purified with flash column chromatography (eluted with a gradient of water/methanol=100:0-0:100) to obtain 310 mg of oily liquid. MS (m/z): 236.4 [M+H]⁺.

### (B) 1-(4-(hydroxymethyl)cyclohexyl)pyridin-2(1H)-one

To a reaction flask were added successively methyl 4-(2-oxopyridin-1(2H)-yl)cyclohexane-1-carboxylate (310 mg, 1.32 mmol) and tetrahydrofuran (10 ml), and the mixture was cooled to −78° C. 1 mole/liter diisobutylaluminium hydride (1.32 ml, 1.32 mmol) was added, and the mixture was reacted at room temperature for 16 hours. After the reaction was completed, the reaction solution was concentrated, and the residue was purified with flash column chromatography (eluted with a gradient of water/methanol=100:0-0:100) to obtain 170 mg of oily liquid. MS (m/z): 208.0 [M+H]⁺.

### (C) 4-(2-oxopyridin-1(2H)-yl)cyclohexane-1-carbaldehyde

To a reaction flask were added successively 1-(4-(hydroxymethyl)cyclohexyl) pyridin-2(1H)-one (170 mg, 0.82 mmol) and dichloromethane (15 ml), followed by Dess-Martin periodinane (383 mg, 0.90 mmol), and the mixture was reacted at room temperature for 3 hours. After the reaction was completed, the reaction solution was concentrated, and the residue was purified with flash column chromatography (eluted with a gradient of water/methanol=100:0-0:100) to obtain 70 mg of oily liquid. MS (m/z): 206.1 [M+H]⁺.

### Intermediate 38: 1-(5-methoxypyridin-2-yl)piperidine-4-carbaldehyde

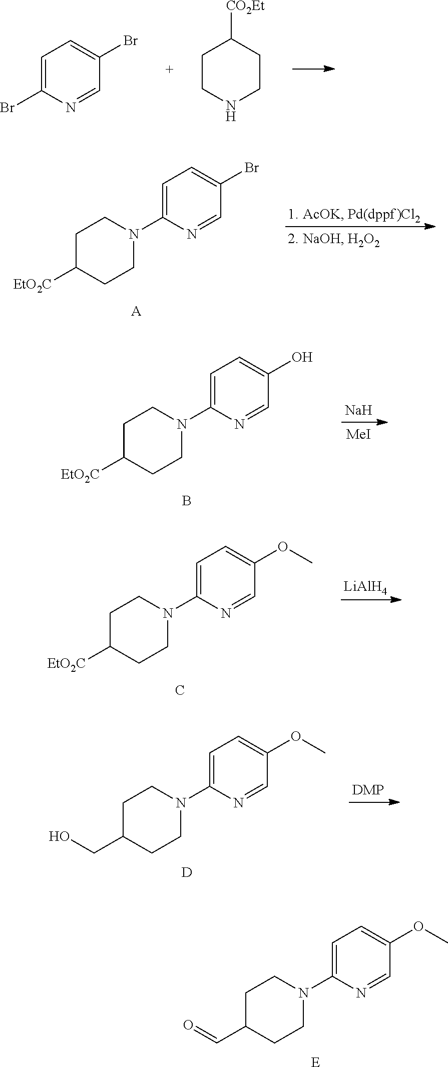

### (A) ethyl 1-(5-bromopyridin-2-yl)piperidine-4-carboxylate

To a reaction flask were added successively 2,5-dibromopyridine (2.37 g, 10 mmol), ethyl piperidine-4-carboxylate (1.57 g, 10 mmol), cesium carbonate (3.26 g, 10 mmol) and N,N-dimethylformamide (30 ml), and the mixture was heated to 110° C. and reacted for 16 hours. After the reaction was completed, water was added, and then the mixture was extracted with ethyl acetate. The organic phase was concentrated, and the residue was purified with flash column chromatography (eluted with a gradient of water/methanol=100:0-0:100) to obtain 2.6 g of oily liquid. MS (m/z): 313.2 [M+H]⁺.

### (B) ethyl 1-(5-hydroxypyridin-2-yl)piperidine-4-carboxylate

To a reaction flask were added successively ethyl 1-(5-bromopyridin-2-yl)piperidine-4-carboxylate (2.6 g, 8.3 mmol), bis(pinacolato)diboron (4.2 g, 16.5 mmol), Pd(dppf)Cl₂·CH₂Cl₂ (607 mg, 0.83 mmol), potassium acetate (1.63 g, 16.6 mmol) and dioxane (40 ml), and the mixture was heated to 100° C. and reacted for 16 hours under nitrogen protection. The reaction solution was concentrated and dissolved in methanol (100 ml) and water (20 ml). Sodium hydroxide (664 mg, 16.6 mmol) and hydrogen peroxide (2 ml) were added at 0′T, and the mixture was reacted at room temperature for 2 hours. After the reaction was completed, the reaction solution was quenched with sodium thiosulfate and extracted with ethyl acetate. The organic phase was concentrated, and the residue was purified with flash column chromatography (eluted with a gradient of water/methanol=100:0-0:100) to obtain 1.1 g of solid. MS (m/z): 251.2 [M+H]⁺.

### (C) ethyl 1-(5-methoxypyridin-2-yl)piperidine-4-carboxylate

To a reaction flask were added successively ethyl 1-(5-hydroxypyridin-2-yl)piperidine-4-carboxylate (1.1 g, 4.39 mmol), sodium hydride (210 mg, 8.75 mmol) and N,N-dimethylformamide (20 ml). Iodomethane (748 mg, 5.27 mmol) was added, and the mixture was reacted at room temperature for 2 hours. After the reaction was completed, water was added, and the mixture was extracted with ethyl acetate. The organic phase was concentrated, and the residue was purified with flash column chromatography (eluted with a gradient of water/methanol=100:0-0:100) to obtain 620 mg of oily liquid. MS (m/z): 265.4 [M+H]⁺.

### (D) (1-(5-methoxypyridin-2-yl)piperidin-4-yl)methanol

To a reaction flask were added successively ethyl 1-(5-methoxypyridin-2-yl)piperidine-4-carboxylate (620 mg, 2.35 mmol) and tetrahydrofuran (10 ml), and the mixture was cooled to 0° C. Lithium aluminum hydride (177 mg, 4.67 mmol) was added, and the mixture was reacted at room temperature for 5 hours. After the reaction was completed, the reaction solution was concentrated, and the residue was purified with flash column chromatography (eluted with a gradient of water/methanol=100:0-0:100) to obtain 370 mg of oily liquid. MS (m/z): 223.4 [M+H]⁺.

### (E) 1-(5-methoxypyridin-2-yl)piperidine-4-carbaldehyde

To a reaction flask were added successively (1-(5-methoxypyridin-2-yl)piperidin-4-yl)methanol (370 mg, 1.66 mmol) and dichloromethane (15 ml), followed by Dess-Martin periodinane (848 mg, 2.00 mmol), and the mixture was reacted at room temperature for 3 hours. After the reaction was completed, the reaction solution was concentrated, and the residue was purified with flash column chromatography (eluted with a gradient of water/methanol=100:0-0:100) to obtain 65 mg of oily liquid. MS (m/z): 221.4 [M+H]⁺.

### Intermediate 39: 5-formyl-N-methylbenzofuran-2-carboxamide

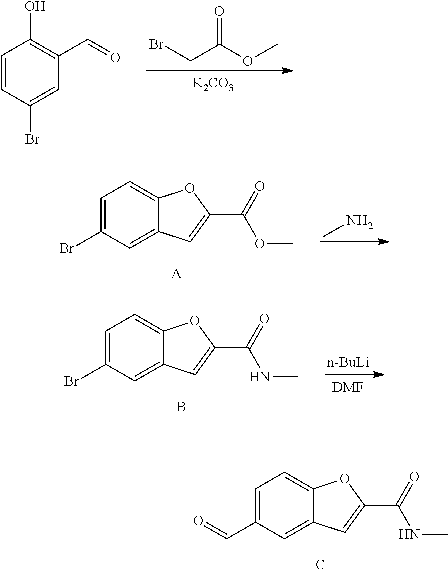

### (A) methyl 5-bromobenzofuran-2-carboxylate

To a reaction flask were added successively 5-bromo-2-hydroxybenzaldehyde (2.01 g, 10 mmol), methyl 2-bromoacetate (2.30 g, 15 mmol), potassium carbonate (2.76 g, 20 mmol) and N,N-dimethylformamide (20 ml), and the mixture was heated to 110° C. and reacted for 5 hours. After the reaction was completed, water was added, and the mixture was extracted with ethyl acetate. The organic phase was concentrated, and the residue was purified with flash column chromatography (eluted with a gradient of water/methanol=100:0-0:100) to obtain 1.6 g of solid. MS (m/z): 255.0 [M+H]⁺.

### (B) 5-bromo-N-methylbenzofuran-2-carboxamide

To a reaction flask were added successively methyl 5-bromobenzofuran-2-carboxylate (765 mg, 3 mmol) and methanamine in methanol (20 ml), and the mixture was heated to 80° C. and reacted for 16 hours. After the reaction was completed, the reaction solution was concentrated, and the residue was purified with flash column chromatography (eluted with a gradient of water/methanol=100:0-0:100) to obtain 762 mg of oily liquid. MS (m/z): 254.2 [M+H]⁺.

### (C) 5-formyl-N-methylbenzofuran-2-carboxamide

To a reaction flask were added 5-bromo-N-methylbenzofuran-2-carboxamide (508 mg, 2 mmol) and anhydrous tetrahydrofuran (20 ml), and the mixture was cooled to −78° C. 1.6 mole/liter n-butyl lithium solution (1.37 ml, 2.2 mmol) was added dropwise, and after 1 hour, N,N-dimethylformamide (0.5 ml) was added. The mixture was then stirred at room temperature for 1 hour. After the reaction was completed, the reaction solution was concentrated, and the residue was purified with flash column chromatography (eluted with a gradient of water/methanol=100:0-0:100) to obtain 180 mg of oily liquid. MS (m/z): 204.2 [M+H]⁺.

### Intermediate 40: 5-formyl-N,3-dimethylbenzofuran-2-carboxamide

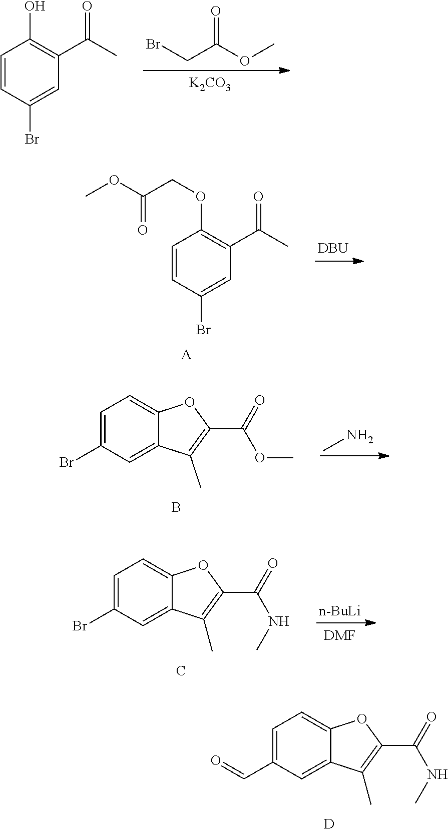

### (A) methyl 2-(2-acetyl-4-bromophenoxy)acetate

To a reaction flask were added successively 1-(5-bromo-2-hydroxylphenyl)ethan-1-one (1.07 g, 5.0 mmol), methyl 2-bromoacetate (765 mg, 5.0 mmol), potassium carbonate (830 mg, 6.0 mmol) and N,N-dimethylformamide (20 ml), and the mixture was heated to 110° C. and reacted for 5 hours. After the reaction was completed, water was added, and the mixture was extracted with ethyl acetate. The organic phase was concentrated, and the residue was purified with flash column chromatography (eluted with a gradient of water/methanol=100:0-0:100) to obtain 980 mg of solid. MS (m/z): 287.2 [M+H]⁺.

### (B) methyl 5-bromo-3-methylbenzofuran-2-carboxylate

To a reaction flask were added successively methyl 2-(2-acetyl-4-bromophenoxy)acetate (980 mg, 3.41 mmol), DBU (1036 mg, 6.81 mmol) and tetrahydrofuran (20 ml), and the mixture was heated to 100° C. and reacted for 2 hours. After the reaction was completed, water was added, and the mixture was extracted with ethyl acetate. The organic phase was concentrated, and the residue was purified with flash column chromatography (eluted with a gradient of water/methanol=100:0-0:100) to obtain 630 mg of solid. MS (m/z): 269.2 [M+H]⁺.

### (C) 5-bromo-N,3-dimethylbenzofuran-2-carboxamide

To a reaction flask were added successively methyl 5-bromo-3-methylbenzofuran-2-carboxylate (630 mg, 2.34 mmol) and methanamine in methanol (20 ml), and the mixture was heated to 80° C. and reacted for 16 hours. After the reaction was completed, the reaction solution was concentrated, and the residue was purified with flash column chromatography (eluted with a gradient of water/methanol=100:0-0:100) to obtain 550 mg of oily liquid. MS (m/z): 270.4 [M+H]⁺.

### (D) 5-formyl-N,3-dimethylbenzofuran-2-carboxamide

To a reaction flask were added 5-bromo-N,3-dimethylbenzofuran-2-carboxamide (534 mg, 2 mmol) and anhydrous tetrahydrofuran (20 ml), and the mixture was cooled to −78° C. 1.6 mole/liter n-butyl lithium solution (1.37 ml, 2.2 mmol) was added dropwise, and after 1 hour, N,N-dimethylformamide (0.5 ml) was added. The mixture was then stirred at room temperature for 1 hour. After the reaction was completed, the reaction solution was concentrated, and the residue was purified with flash column chromatography (eluted with a gradient of water/methanol=100:0-0:100) to obtain 260 mg of oily liquid. MS (m/z): 218.2 [M+H]⁺.

### Example 2

### Preparation of compounds 1-184

### Compound 1: 2-((5-(7-((1-(cyclopropanecarbonyl)piperidin-4-yl)methyl)-2,7-diazaspiro[3.5]nonan-2-yl)-1,2,4-triazin-6-yl)oxy)-N-ethyl-5-fluoro-N-isopropylbenzamide

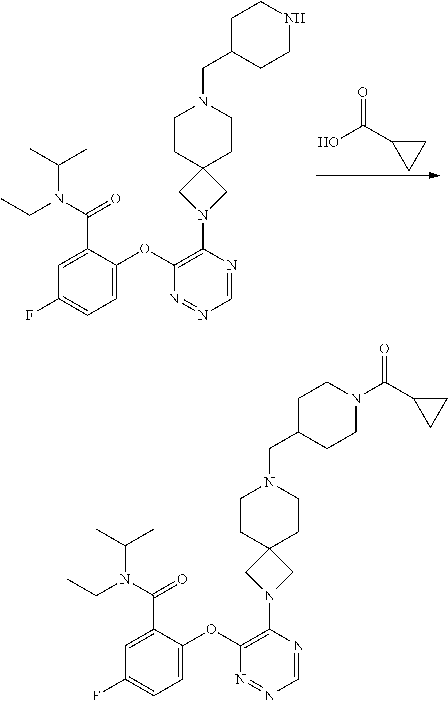 [SMILES: CCN(C(=O)c1cc(F)ccc1Oc1nncnc1N1CC2(CCN(CC3CCNCC3)CC2)C1)C(C)C ; O=C(O)C1CC1 ; CCN(C(=O)c1cc(F)ccc1Oc1nncnc1N1CC2(CCN(CC3CCN(C(=O)C4CC4)CC3)CC2)C1)C(C)C]

To a reaction flask were added successively intermediate 10-1 (40 mg, 0.076 mmol), cyclopropane carboxylic acid (10 mg, 0.116 mmol), HATU (43 mg, 0.113 mmol), N,N-diisopropylethylamine (29 mg, 0.224 mmol) and dichloromethane (5 ml). The mixture was reacted at room temperature for 16 hours, and the reaction solution was concentrated to remove the solvent. The residue was then purified with flash column chromatography (eluted with a gradient of water/methanol=100:0-0:100) to obtain 22 mg of white solid. MS (m/z): 594.4 [M+H]⁺.

¹H NMR (400 MHz, DMSO-d6) δ 8.43 (d, J=0.4 Hz, 1H), 7.42 (dd, J=6.1, 3.5 Hz, 1H), 7.35-7.31 (m, 2H), 4.29 (s, 1H), 4.19 (s, 3H), 3.78 (s, 2H), 3.64-3.55 (m, 1H), 3.08-2.97 (m, 2H), 2.31 (s, 4H), 2.06 (d, J=6.9 Hz, 2H), 2.00-1.87 (m, 2H), 1.76-1.62 (m, 8H), 1.10-1.00 (m, 6H), 0.95-0.90 (m, 3H), 0.68-0.62 (m, 6H).

The following compounds were prepared according to the preparation procedure of compound 1 using corresponding intermediates and reagents under appropriate conditions that will be recognized by those skilled in the art.

| Com- | | MS | | Intermediate |
|---|---|---|---|---|
| pound | Structural formula | (M + H)⁺ | ¹HNMR | used |
| 2 | 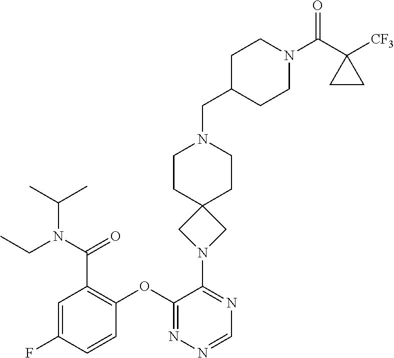 [SMILES: CCN(C(=O)c1cc(F)ccc1Oc1nncnc1N1CC2(CCN(CC3CCN(C(=O)C4(C(F)(F)F)CC4)CC3)CC2)C1)C(C)C] | 662.4 | ¹H NMR (400 MHz, CD₃OD) δ 8.35 (s, 1H), 7.37 (dd, J = 9.0, 4.4 Hz, 1H), 7.23 (td, J = 8.7, 3.0 Hz, 1H), 7.13 (dd, J = 8.0, 2.9 Hz, 1H), 4.45- 4.27 (m, 4H), 4.26-4.14 (m, 1H), 3.97-3.86 (m, 2H), 3.85- 3.77 (m, 1H), 3.52-3.35 (m, 1H), 3.20-3.04 (m, 1H), 3.02- 2.80 (m, 2H), 2.47-2.34 (m, 4H), 2.19 (d, J = 6.5 Hz, 2H), 1.87-1.81 (m, 7H), 1.33- 1.29 (m, 2H), 1.24-1.19 (m, 2H), 1.18-1.14 (m, 4H), 1.12- 1.05 (m, 3H), 1.05-1.01 (m, 1H), 0.94-0.76 (m, 2H). | 10-1 |
| 3 | 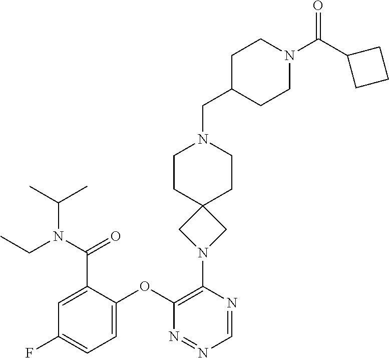 [SMILES: CCN(C(=O)c1cc(F)ccc1Oc1nncnc1N1CC2(CCN(CC3CCN(C(=O)C4CCC4)CC3)CC2)C1)C(C)C] | 608.4 | ¹H NMR (400 MHz, DMSO-d₆) δ 8.43 (d, J = 1.0 Hz, 1H), 7.45- 7.40 (m, 1H), 7.34 (t, J = 7.0 Hz, 2H), 4.29 (d, J = 13.4 Hz, 1H), 4.18 (s, 2H), 3.78 (s, 2H), 3.60 (d, J = 5.8 Hz, 2H), 3.28- 3.22 (m, 1H), 3.05-3.01 (m, 1H), 2.85 (t, J = 12.4 Hz, 1H), 2.26-2.21 (m, 4H), 2.14-2.07 (m, 3H), 2.03 (d, J = 6.3 Hz, 4H), 1.91-1.75 (m, 2H), 1.68- 1.64 (m, 8H), 1.06-0.97 (m, 6H), 0.89-0.85 (m, 3H), 0.69 (d, J = 6.0 Hz, 2H). | 10-1 |
| 4 | 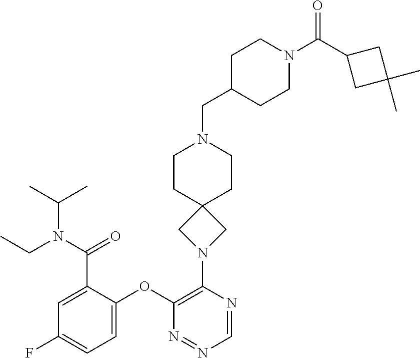 [SMILES: CCN(C(=O)c1cc(F)ccc1Oc1nncnc1N1CC2(CCN(CC3CCN(C(=O)C4CC(C)(C)C4)CC3)CC2)C1)C(C)C] | 636.8 | ¹H NMR (400 MHz, CD₃OD) δ 8.37 (s, 1H), 7.42-7.37 (m, 1H), 7.29-7.24 (m, 1H), 7.23- 7.17 (m, 1H), 4.48-4.44 (m, 1H), 4.40-4.30 (m, 2H), 3.95- 3.86 (m, 2H), 3.83-3.75 (m, 2H), 3.53-3.46 (m, 1H), 3.27- 3.24 (m, 1H), 3.23-3.20 (m, 1H), 2.97 (t, J = 11.9 Hz, 1H), 2.64-2.57 (m, 1H), 2.50- 2.25 (br, 4H), 2.17 (d, J = 6.6 Hz, 2H), 2.02-1.98 (m, 2H), 1.96-1.88 (m, 2H), 1.87-1.82 (m, 5H), 1.80-1.76 (m, 2H), 1.22-1.20 (m, 1H), 1.19 (s, 3H), 1.16-1.11 (m, 5H), 1.09- 1.06 (m, 1H), 1.04 (s, 3H), 1.03-0.96 (m, 2H), 0.81-0.78 (m, 2H). | 10-1 |
| 5 | 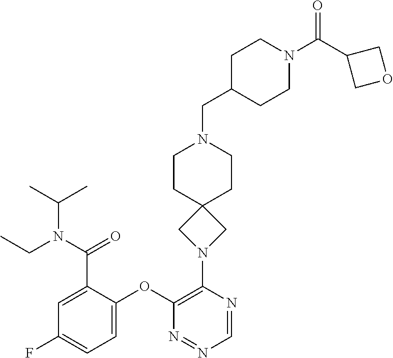 [SMILES: CCN(C(=O)c1cc(F)ccc1Oc1nncnc1N1CC2(CCN(CC3CCN(C(=O)C4COC4)CC3)CC2)C1)C(C)C] | 610.4 | ¹H NMR (400 MHz, CD₃OD) δ 8.38 (s, 1H), 7.43-7.38 (m, 1H), 7.32-7.17 (m, 2H), 5.41 (s, 1H), 5.14 (s, 1H), 4.53- 4.48 (m, 1H), 4.40-4.32 (m, 2H), 4.25-4.17 (m, 2H), 4.13-4.08 (m, 1H), 3.94-3.90 (m, 2H), 3.84-3.79 (m, 1H), 3.55-3.43 (m, 1H), 3.28-3.16 (m, 1H), 3.13-3.07 (m, 1H), 2.77-2.70 (m, 1H), 2.58-2.22 (m, 4H), 2.20 (d, J = 6.7 Hz, 2H), 1.90-1.78 (m, 7H), 1.21- 1.19 (m, 2H), 1.16-1.12 (m, 6H), 1.08-1.04 (m, 1H), 0.82-0.77 (m, 2H). | 10-1 |
| 6 | 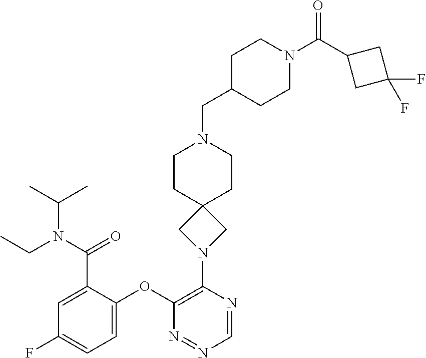 [SMILES: CCN(C(=O)c1cc(F)ccc1Oc1nncnc1N1CC2(CCN(CC3CCN(C(=O)C4CC(F)(F)C4)CC3)CC2)C1)C(C)C] | 644.4 | ¹H NMR (400 MHz, CD₃OD) δ 8.44 (s, 1H), 8.41 (s, 1H), 7.43-7.38 (m, 1H), 7.33-7.19 (m, 2H), 4.51 (d, J = 13.4 Hz, 1H), 4.45-4.38 (m, 2H), 4.06- 3.95 (m, 2H), 3.92-3.76 (m, 2H), 3.58-3.41 (m, 1H), 3.28- 3.17 (m, 2H), 3.11-3.05 (m, 2H), 3.02-2.87 (m, 3H), 2.85- 2.73 (m, 4H), 2.72-2.67 (m, 3H), 2.10-2.03 (m, 5H), 1.87- 1.81 (m, 2H), 1.21-1.17 (m, 2H), 1.16-1.11 (m, 6H), 1.07 (t, J = 7.2 Hz, 1H), 0.82- 0.76 (m, 2H). | 10-1 |
| 7 | 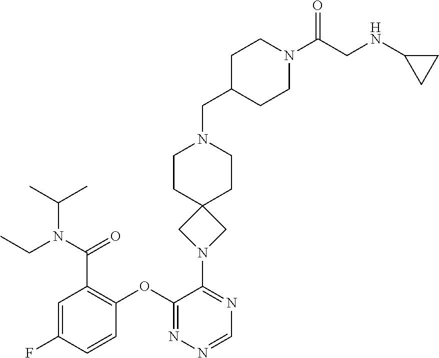 [SMILES: CCN(C(=O)c1cc(F)ccc1Oc1nncnc1N1CC2(CCN(CC3CCN(C(=O)CNC4CC4)CC3)CC2)C1)C(C)C] | 623.4 | ¹H NMR (400 MHz, CD₃OD) δ 8.37 (s, 1H), 7.44-7.36 (m, 1H), 7.32-7.17 (m, 2H), 4.52- 4.43 (m, 1H), 4.40-4.28 (m, 2H), 3.96-3.77 (m, 4H), 3.52- 3.49 (m, 2H), 3.26-3.15 (m, 1H), 3.03 (t, J = 12.0 Hz, 1H), 2.66 (t, J = 12.1 Hz, 1H), 2.57-2.25 (m, 4H), 2.23-2.16 (m, 3H), 1.90-1.77 (m, 7H), 1.34-1.26 (m, 1H), 1.24-1.17 (m, 2H), 1.17-1.08 (m, 6H), 1.08-0.99 (m, 2H), 0.79 (d, J = 6.2 Hz, 2H), 0.48-0.42 (m, 2H), 0.39-0.33 (m, 2H). | 10-1 |
| 8 | 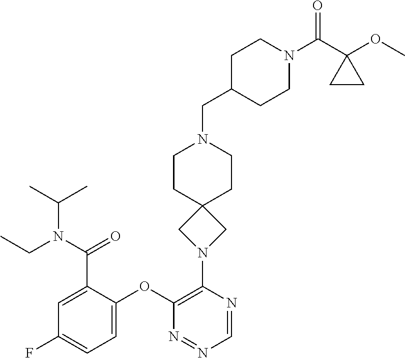 [SMILES: CCN(C(=O)c1cc(F)ccc1Oc1nncnc1N1CC2(CCN(CC3CCN(C(=O)C4(OC)CC4)CC3)CC2)C1)C(C)C] | 624.4 | ¹H NMR (400 MHz, CD₃OD) δ 8.47 (s, 1H), 8.39 (s, 1H), 7.41-7.37 (m, 1H), 7.30-7.24 (m, 1H), 7.22-7.19 (m, 1H), 4.60-4.35 (m, 4H), 4.01-3.92 (m, 2H), 3.84-3.78 (m, 1H), 3.55-3.40 (m, 1H), 3.28 (s, 3H), 3.26-3.16 (m, 1H), 3.16-2.91 (m, 2H), 2.89-2.71 (br, 4H), 2.58 (d, J = 6.9 Hz, 2H), 2.05-1.97 (m, 5H), 1.85 (d, J = 12.5 Hz, 2H), 1.24- 1.18 (m, 3H), 1.17-1.11 (m, 5H), 1.06 (t, J = 7.1 Hz, 1H), 1.02-0.96 (m, 2H), 0.95-0.90 (m, 2H), 0.83-0.77 (m, 2H). | 10-1 |
| 9 | 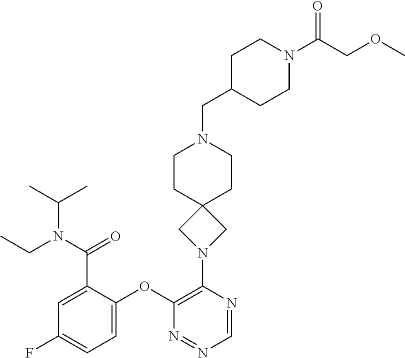 [SMILES: CCN(C(=O)c1cc(F)ccc1Oc1nncnc1N1CC2(CCN(CC3CCN(C(=O)COC)CC3)CC2)C1)C(C)C] | 598.4 | ¹H NMR (400 MHz, CD₃OD) δ 8.41(s, 1H), 7.42-7.39 (m, 1H), 7.33-7.16 (m, 2H), 4.56- 4.36 (m, 3H), 4.19-4.08 (m, 2H), 4.02-3.98 (m, 2H), 3.93- 3.76 (m, 2H), 3.49-3.46 (m, 1H), 3.38 (s, 3H), 3.07-3.03 (m, 6H), 2.76-2.71 (m, 3H), 2.09-2.04 (m, 5H), 1.85-1.81 (m, 2H), 1.26-1.04 (m, 9H), 0.83-0.78 (m, 2H). | 10-1 |
| 10 | 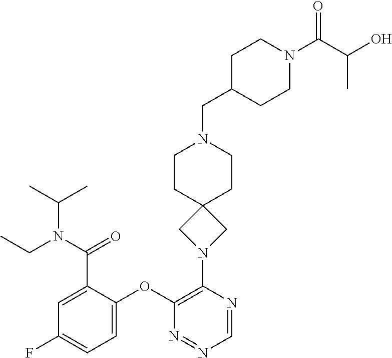 [SMILES: CCN(C(=O)c1cc(F)ccc1Oc1nncnc1N1CC2(CCN(CC3CCN(C(=O)C(C)O)CC3)CC2)C1)C(C)C] | 598.4 | ¹H NMR (400 MHz, CD₃OD) δ 8.39 (s, 1H), 7.42-7.39 (m, 1H), 7.33-7.18 (m, 2H), 4.57- 4.53 (m, 2H), 4.46-4.43 (m, 2H), 4.07-4.03 (m, 3H), 3.93- 3.76 (m, 1H), 3.48-3.45 (m, 1H), 3.15-3.10 (m, 6H), 2.91-2.87 (m, 2H), 2.73-2.69 (m, 1H), 2.13-2.08 (m, 5H), 1.88-1.83 (m, 2H), 1.56-1.36 (m, 2H), 1.32-1.28 (m, 3H), 1.22-1.06 (m, 8H), 0.83- 0.78 (m, 2H). | 10-1 |
| 11 | 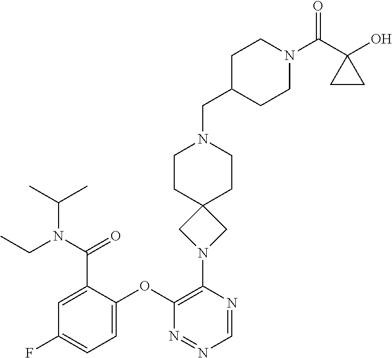 [SMILES: CCN(C(=O)c1cc(F)ccc1Oc1nncnc1N1CC2(CCN(CC3CCN(C(=O)C4(O)CC4)CC3)CC2)C1)C(C)C] | 610.4 | ¹H NMR (400 MHz, CD₃OD) δ 8.42 (s, 1H), 8.34 (s, 1H), 7.44-7.41 (m, 1H), 7.33-7.18 (m, 2H), 4.48-4.43 (m, 4H), 4.05-4.02 (m, 2H), 3.88- 3.77 (m, 1H), 3.49-3.46 (m, 1H), 3.29-3.09 (m, 5H), 2.99- 2.96 (m, 3H), 2.17-2.13 (m, 5H), 1.86-1.83 (m, 2H), 1.36- 1.04 (m, 10H), 1.03-1.00 (m, 2H), 0.90-0.75 (m, 4H). | 10-1 |
| 12 | 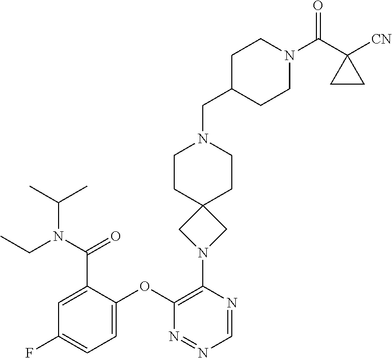 [SMILES: CCN(C(=O)c1cc(F)ccc1Oc1nncnc1N1CC2(CCN(CC3CCN(C(=O)C4(C#N)CC4)CC3)CC2)C1)C(C)C] | 619.4 | ¹H NMR (400 MHz, CD₃OD) δ 8.41 (s, 1H), 8.27 (s, 1H), 7.42-7.38 (m, 1H), 7.33-7.16 (m, 2H), 4.44-4.41 (m, 4H), 4.03-4.39 (m, 2H), 3.88- 3.75 (m, 1H), 3.49-3.45 (m, 1H), 3.25-3.20 (m, 5H), 2.99- 2.95 (m, 2H), 2.87-2.84 (m, 1H), 2.15-2.10 (m, 5H), 1.91- 1.88 (m, 2H), 1.58-1.53 (m, 5H), 1.29-1.02 (m, 9H), 0.82-0.77 (m, 2H). | 10-1 |
| 13 | 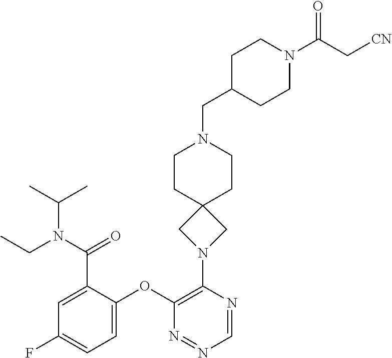 [SMILES: CCN(C(=O)c1cc(F)ccc1Oc1nncnc1N1CC2(CCN(CC3CCN(C(=O)CC#N)CC3)CC2)C1)C(C)C] | 593.6 | ¹H NMR (400 MHz, CD₃OD) δ 8.38 (s, 1H), 7.45-7.37 (m, 1H), 7.32-7.17 (m, 2H), 4.51- 4.15 (m, 4H), 3.98-3.87 (m, 2H), 3.85-3.68 (m, 2H), 3.56-3.42 (m, 1H), 3.28-3.17 (m, 1H), 3.15-3.07 (m, 1H), 2.75-2.63 (m, 1H), 2.59-2.25 (m, 4H), 2.20 (d, J = 6.6 Hz, 2H), 2.03-1.75 (m, 8H), 1.21 (d, J = 6.7 Hz, 2H), 1.18-1.12 (m, 5H), 1.06 (t, J = 7.1 Hz, 2H), 0.80 (d, J = 6.3 Hz, 2H). | 10-1 |
| 14 | 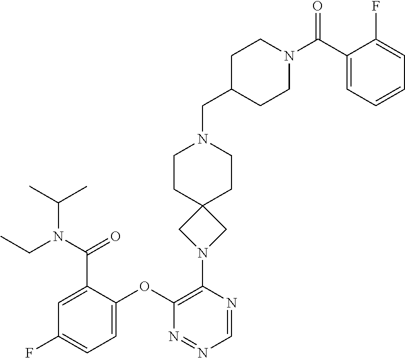 [SMILES: CCN(C(=O)c1cc(F)ccc1Oc1nncnc1N1CC2(CCN(CC3CCN(C(=O)c4ccccc4F)CC3)CC2)C1)C(C)C] | 648.4 | ¹H NMR (400 MHz, CD₃OD) δ 8.37 (s, 1H), 7.51-7.45 (m, 1H), 7.44-7.32 (m, 2H), 7.31- 7.23 (m, 2H), 7.23-7.14 (m, 2H), 4.65 (d, J = 13.1 Hz, 1H), 4.44-4.23 (m, 2H), 3.97- 3.86 (m, 2H), 3.85-3.78 (m, 1H), 3.60-3.42 (m, 2H), 3.26- 3.03 (m, 2H), 2.87 (t, J = 11.8 Hz, 1H), 2.55-2.26 (br, 4H), 2.25-2.18 (m, 2H), 1.95- 1.82 (m, 6H), 1.78-1.70 (m, 1H), 1.32-1.26 (m, 1H), 1.22-1.18 (m, 2H), 1.17-1.10 (m, 5H), 1.06 (t, J = 7.1 Hz, 1H), 0.82-0.78 (m, 2H) | 10-1 |
| 15 | 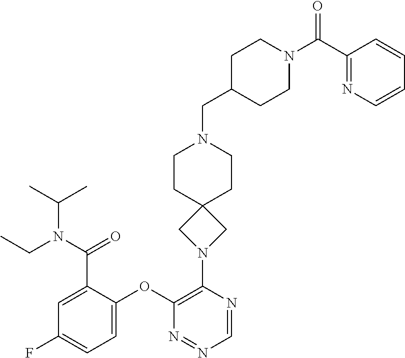 [SMILES: CCN(C(=O)c1cc(F)ccc1Oc1nncnc1N1CC2(CCN(CC3CCN(C(=O)c4ccccn4)CC3)CC2)C1)C(C)C] | 631.4 | ¹H NMR (400 MHz, CD₃OD) δ 8.59-8.56 (m, 1H), 8.37 (s, 1H), 7.97-7.93 (m, 1H), 7.56- 7.53 (m, 1H), 7.50-7.46 (m, 1H), 7.43-7.38 (m, 1H), 7.31- 7.23 (m, 1H), 7.23-7.18 (m, 1H), 4.63 (d, J = 13.1 Hz, 1H), 4.40-4.25 (m, 2H), 3.95- 3.87 (m, 2H), 3.86-3.76 (m, 1H), 3.66-3.60 (m, 1H), 3.57 -3.43 (m, 1H), 3.27-3.15 (m, 1H), 3.14-3.06 (m, 1H), 2.95- 2.83 (m, 1H), 2.55-2.26 (br, 4H), 2.23-2.19 (m, 2H), 1.94- 1.83 (m, 6H), 1.72 (d, J = 12.7 Hz, 1H), 1.28-1.23 (m, 1H), 1.22-1.19 (m, 2H), 1.17- 1.12 (m, 5H), 1.06 (t, J = 7.1 Hz, 1H), 0.83-0.78 (m, 2H). | 10-1 |
| 16 | 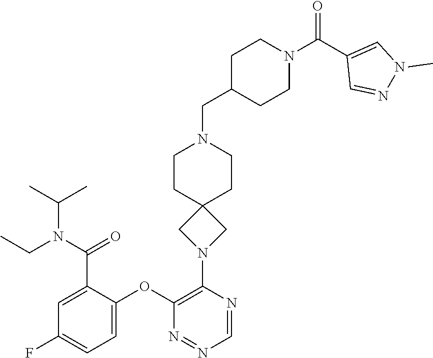 [SMILES: CCN(C(=O)c1cc(F)ccc1Oc1nncnc1N1CC2(CCN(CC3CCN(C(=O)c4cnn(C)c4)CC3)CC2)C1)C(C)C] | 634.4 | ¹H NMR (400 MHz, CD₃OD) δ 8.42 (s, 1H), 7.93 (s, 1H), 7.68 (s, 1H), 7.43-7.36 (m, 1H), 7.33-7.19 (m, 2H), 4.68- 4.39 (m, 3H), 4.30-4.13 (m, 1H), 4.08-3.96 (m, 2H), 3.91 (s, 3H), 3.87-3.76 (m, 1H), 3.53-3.41 (m, 1H), 3.27-2.97 (m, 6H), 2.92-2.86 (m, 2H), 2.26-2.05 (m, 5H), 1.92-1.80 (m, 2H), 1.35-1.21 (m, 3H), 1.20-1.04 (m, 7H), 0.85-1.73 (m, 2H). | 10-1 |
| 17 | 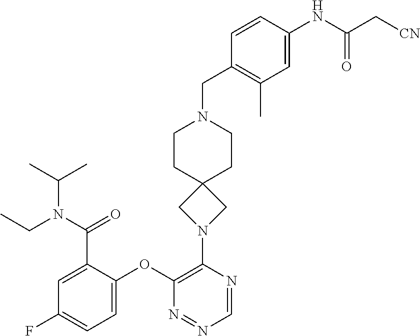 [SMILES: CCN(C(=O)c1cc(F)ccc1Oc1nncnc1N1CC2(CCN(Cc3ccc(NC(=O)CC#N)cc3C)CC2)C1)C(C)C] | 615.4 | ¹H NMR (400 MHz, CD₃OD) δ 8.40 (s, 1H), 7.46-7.36 (m, 3H), 7.34-7.17 (m, 3H), 4.42 (s, 2H), 3.98 (d, J = 7.1 Hz, 2H), 3.90 (s, 2H), 3.86-3.74 (m, 1H), 3.54-3.41 (m, 1H), 3.29 (s, 2H), 3.28-3.15 (m, 1H), 3.06-2.75 (m, 4H), 2.39 (s, 3H), 2.05-1.92 (br, 4H), 1.23-1.03 (m, 7H), 0.79 (d, J = 6.1 Hz, 2H). | 10-2 |

### Compound 18: 2-((5-(7-((1-(1-acryloylazetidine-3-carbonyl)piperidin-4-yl)methyl)-2,7-diazaspiro[3.5]nonan-2-yl)-1,2,4-triazin-6-yl)oxy)-N-ethyl-5-fluoro-N-isopropylbenzamide

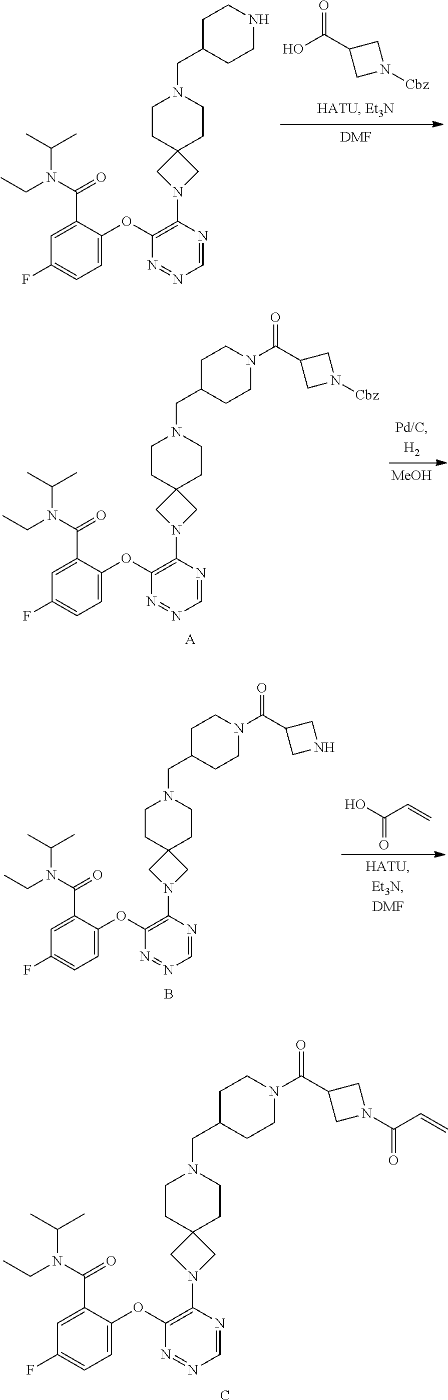

### (A) benzyl 3-(4-((2-(6-(2-(ethyl(isopropyl)carbamoyl)-4-fluorophenoxy)-1,2,4-triazin-5-yl)-2,7-diazaspiro[3.5]nonan-7-yl)methyl)piperidine-1-carbonyl)azetidine-1-carboxylate

To a reaction flask were added successively intermediate 10-1 (150 mg, 0.29 mmol), 1-((benzyloxy)carbonyl)azetidine-3-carboxylic acid (106 mg, 0.45 mmol), HATU (132 mg, 0.35 mmol), triethylamine (90 mg, 0.90 mmol) and N,N-dimethylformamide (3 ml), and the mixture was reacted at room temperature for 2 hours. The reaction solution was purified with flash column chromatography (eluted with a gradient of water/methanol=100:0-0:100) to obtain 220 mg of light yellow solid. MS (m/z): 743.4 [M+H]⁺.

### (B) 2-((5-(7-((1-(azetidine-3-carbonyl)piperidin-4-yl)methyl)-2,7-diazaspiro[3.5]nonan-2-yl)-1,2,4-triazin-6-yl)oxy)-N-ethyl-5-fluoro-N-isopropylbenzamide

To a reaction flask were added successively benzyl 3-(4-((2-(6-(2-(ethyl(isopropyl) carbamoyl)-4-fluorophenoxy)-1,2,4-triazin-5-yl)-2,7-diazaspiro[3.5]nonan-7-yl)methyl) piperidine-1-carbonyl)azetidine-1-carboxylate (220 mg, 0.30 mmol), methanol (5 ml) and palladium carbon (22 mg), and the mixture was reacted under hydrogen at room temperature for 15 hours. The reaction solution was filtered, and the filtrate was concentrated to obtain 140 mg of crude product, which was directly used in the next reaction. MS (m/z): 609.2 [M+H]⁺.

### (C) 2-((5-(7-((1-(1-acryloylazetidine-3-carbonyl)piperidin-4-yl)methyl)-2,7-diazaspiro[3.5]nonan-2-yl)-1,2,4-triazin-6-yl)oxy)-N-ethyl-5-fluoro-N-isopropylbenzamide

To a reaction flask were added successively 2-((5-(7-((1-(azetidine-3-carbonyl) piperidin-4-yl)methyl)-2,7-diazaspiro[3.5]nonan-2-yl)-1,2,4-triazin-6-yl)oxy)-N-ethyl-5-fluoro-N-isopropylbenzamide (70 mg, 0.12 mmol), acrylic acid (17 mg, 0.24 mmol), HATU (54 mg, 0.14 mmol), triethylamine (33 mg, 0.33 mmol) and N,N-dimethylformamide (3 ml), and the mixture was reacted at room temperature for 2 hours. The reaction solution was purified with flash column chromatography (eluted with a gradient of water/methanol=100:0-0:100) to obtain 13.7 mg of light yellow solid. MS (m/z): 663.4 [M+H]⁺.

¹H NMR (400 MHz, CD₃OD) δ 8.38 (s, 1H), 7.42-7.40 (m, 1H), 7.31-7.15 (m, 2H), 6.32-6.29 (m, 1H), 6.25-6.22 (m, 1H), 5.76-5.73 (m, 1H), 4.48-4.45 (m, 3H), 4.38-4.35 (m, 2H), 4.27-4.24 (m, 1H), 4.17-4.14 (m, 1H), 3.97-3.88 (m, 2H), 3.82-3.78 (m, 2H), 3.68-3.65 (m, 1H), 3.49-3.46 (m, 1H), 3.25-3.23 (m, 1H), 3.09-3.05 (m, 1H), 2.72-2.68 (m, 1H), 2.55-2.49 (m, 4H), 2.28-2.25 (m, 2H), 1.89-1.83 (m, 7H), 1.21-1.02 (m, 9H), 0.82-0.78 (m, 2H).

### Compound 19: 2-((5-(7-((1-(1-(dimethylcarbamoyl)cyclopropane-1-carbonyl)piperidin-4-yl)methyl)-2,7-diazaspiro[3.5]nonan-2-yl)-1,2,4-triazin-6-yl)oxy)-N-ethyl-5-fluoro-N-isopropylbenzamide

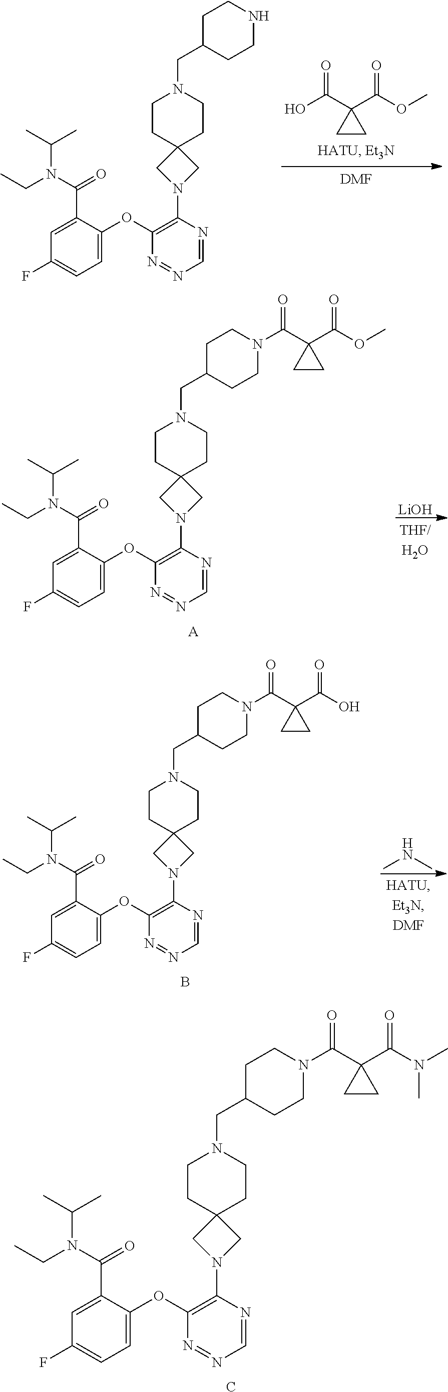

### (A) methyl 1-(4-((2-(6-(2-(ethyl(isopropyl)carbamoyl)-4-fluorophenoxy)-1,2,4-triazin-5-yl)-2,7-diazaspiro[3.5]nonan-7-yl)methyl)piperidine-1-carbonyl)cyclopropane-1-carboxylate

To a reaction flask were added successively intermediate 10-1 (80 mg, 0.15 mmol), 1-(methyloxycarbonyl)cyclopropane-1-carboxylic acid (36 mg, 0.25 mmol), HATU (68 mg, 0.18 mmol), triethylamine (45 mg, 0.45 mmol) and N,N-dimethylformamide (3 ml), and the mixture was reacted at room temperature for 2 hours. The reaction solution was purified with flash column chromatography (eluted with a gradient of water/methanol=100:0-0:100) to obtain 69 mg of light yellow solid. MS (m/z): 652.4 [M+H]⁺.

### (B) 1-(4-((2-(6-(2-(ethyl(isopropyl)carbamoyl)-4-fluorophenoxy)-1,2,4-triazin-5-yl)-2,7-diazaspiro[3.5]nonan-7-yl)methyl)piperidine-1-carbonyl)cyclopropane-1-carboxylic acid

To a reaction flask were added successively methyl 1-(4-((2-(6-(2-(ethyl(isopropyl) carbamoyl)-4-fluorophenoxy)-1,2,4-triazin-5-yl)-2,7-diazaspiro[3.5]nonan-7-yl)methyl) piperidine-1-carbonyl)cyclopropane-1-carboxylate (69 mg, 0.11 mmol), lithium hydroxide monohydrate (14 mg, 0.33 mmol), tetrahydrofuran (4 ml) and water (1 ml), and the mixture was reacted at room temperature for 15 hours. The reaction solution was adjusted to pH 5-6 with 1 mole/liter dilute hydrochloric acid, and concentrated to obtain a crude product, which was purified with flash column chromatography (eluted with a gradient of water/methanol=100:0-0:100) to obtain 47 mg of white solid. MS (m/z): 638.4 [M+H]⁺.

### (C) 2-((5-(7-((1-(1-(dimethylcarbamoyl)cyclopropane-1-carbonyl)piperidin-4-yl)methyl)-2,7-diazaspiro[3.5]nonan-2-yl)-1,2,4-triazin-6-yl)oxy)-N-ethyl-5-fluoro-N-isopropylbenzamide

To a reaction flask were added successively 1-(4-((2-(6-(2-(ethyl(isopropyl) carbamoyl)-4-fluorophenoxy)-1,2,4-triazin-5-yl)-2,7-diazaspiro[3.5]nonan-7-yl) methyl)piperidine-1-carbonyl)cyclopropane-1-carboxylic acid (47 mg, 0.074 mmol), dimethylamine hydrochloride (12 mg, 0.15 mmol), HATU (32 mg, 0.084 mmol), triethylamine (21 mg, 0.21 mmol) and N,N-dimethylformamide (3 ml), and the mixture was reacted at room temperature for 2 hours. The reaction solution was purified with flash column chromatography (eluted with a gradient of water/methanol=100:0-0:100) to obtain 11.3 mg of light yellow solid. MS (m/z): 665.6 [M+H]⁺.

¹H NMR (400 MHz, CD₃OD) δ 8.43 (s, 1H), 8.41 (s, 1H), 7.43-7.40 (m, 1H), 7.27-7.23 (m, 2H), 4.45-4.42 (m, 3H), 4.27-4.23 (m, 1H), 4.01-3.98 (m, 2H), 3.84-3.79 (m, 1H), 3.49-3.46 (m, 1H), 3.22-3.18 (m, 1H), 3.09 (s, 3H), 2.99-2.96 (m, 4H), 2.93 (s, 3H), 2.72-2.68 (m, 3H), 2.07-2.02 (m, 5H), 1.82-1.78 (m, 2H), 1.31-1.04 (m, 14H), 0.82-0.78 (m, 2H).

### Compound 20: 2-((5-(7-((1-acryloylpiperidin-4-yl)methyl)-2,7-diazaspiro[3.5]nonan-2-yl)-1,2,4-triazin-6-yl)oxy)-N-ethyl-5-fluoro-N-isopropylbenzamide

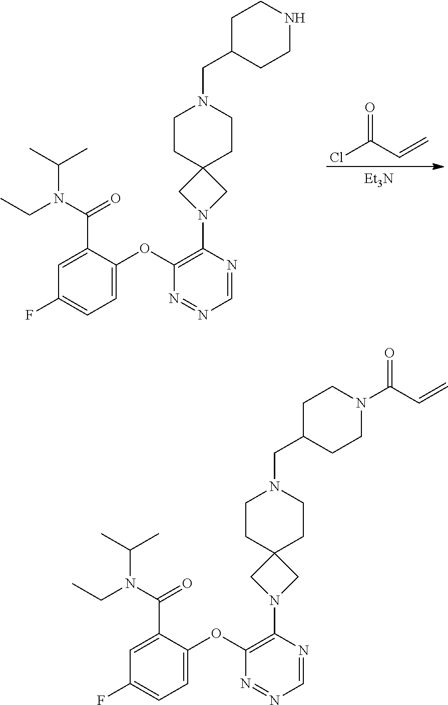

To a reaction flask were added successively intermediate 10-1 (53 mg, 0.1 mmol), triethylamine (0.5 ml) and dichloromethane (5 ml), acryloyl chloride (19 mg, 0.2 mmol) was added at 0° C., and the mixture was reacted at room temperature for 2 hours. After the reaction was completed, the reaction solution was concentrated, and the residue was purified with flash column chromatography (eluted with a gradient of water/methanol=100:0-0:100) to obtain 5 mg of white solid. MS (m/z): 580.3 [M+H]⁺.

¹H NMR (400 MHz, CD₃OD) δ 8.36 (s, 1H), 7.37 (dd, J=9.2, 4.5 Hz, 1H), 7.23 (t, J=8.5 Hz, 1H), 7.14 (d, J=8.1 Hz, 1H), 6.70 (dd, J=16.9, 10.7 Hz, 1H), 6.12 (dd, J=16.9, 1.9 Hz, 1H), 5.67 (dd, J=10.7, 1.9 Hz, H), 4.43-4.14 (m, 2H), 4.11-3.90 (m, 2H), 3.89-3.75 (m, 1H), 3.48-3.36 (m, 1H), 3.18-3.04 (m, 1H), 2.84-2.67 (m, 1H), 2.62-2.42 (m, 4H), 2.31 (d, J=6.7 Hz, 2H), 1.92-1.80 (m, 7H), 1.40-1.24 m, 3H), 1.24-0.99 (m, 9H), 0.97-0.76 (m, 2H).

The following compounds were prepared according to the preparation procedure of compound 20 using corresponding intermediates and reagents under appropriate conditions that will be recognized by those skilled in the art.

| | | MS | | Intermediate |
|---|---|---|---|---|
| Compound | Structural formula | (M + H)⁺ | ¹HNMR | used |
| 21 | 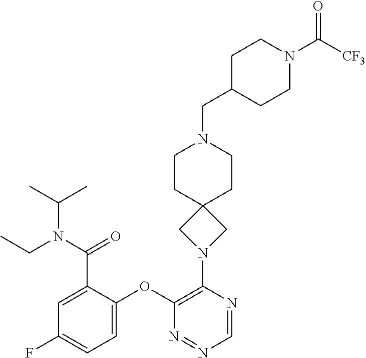 [SMILES: CCN(C(=O)c1cc(F)ccc1Oc1nncnc1N1CC2(CCN(CC3CCN(C(=O)C(F)(F)F)CC3)CC2)C1)C(C)C] | 622.3 | ¹H NMR (400 MHz, DMSO-d₆) δ 8.43 (d, J = 0.7 Hz, 1H), 7.45- 7.40 (m, 1H), 7.33 (dd, J = 8.0, 2.6 Hz, 2H), 4.26-4.16 (m, 4H), 3.78 (s, 2H), 3.63-3.55 (m, 1H), 3.18 (d, J = 12.3 Hz, 1H), 3.08-3.02 (m, 1H), 2.84 (t, J = 12.0 Hz, 1H), 2.30 (s, 4H), 2.07 (d, J = 6.9 Hz, 2H), 1.83-1.68 (m, 8H), 1.10-1.02 (m, 6H), 0.95-0.91 (m, 3H), 0.69 (d, J = 6.3 Hz, 2H). | 10-1 |
| 22 | 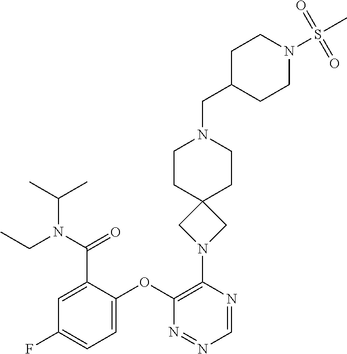 [SMILES: CCN(C(=O)c1cc(F)ccc1Oc1nncnc1N1CC2(CCN(CC3CCN(S(C)(=O)=O)CC3)CC2)C1)C(C)C] | 604.6 | ¹H NMR (400 MHz, CD₃OD) δ 8.38 (s, 1H), 7.44-7.37 (m, 1H), 7.31-7.17 (m, 2H), 4.44-4.30 (m, 2H), 3.98-3.87 (m, 2H), 3.84-3.76 (m, 1H), 3.74-3.65 (m, 2H), 3.55-3.44 (m, 1H), 3.28-3.16 (m, 1H), 2.80 (s, 3H), 2.75-2.67 (m, 2H), 2.23 (d, J = 7.0 Hz, 2H), 1.88-1.84 (m, 5H), 1.75-1.64 (m, 1H), 1.32-1.23 (m, 2H), 1.22-1.19 (m, 2H), 1.17-1.13 (m, 2H), 1.06 (t, J = 7.1 Hz, 1H), 0.80 (d, J = 6.1 Hz, 2H). | 10-1 |
| 23 | 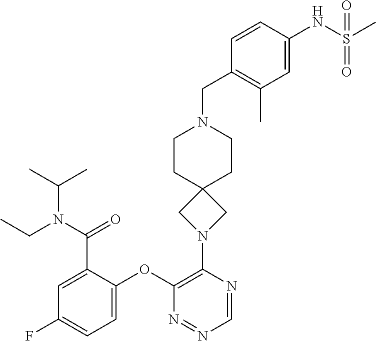 [SMILES: CCN(C(=O)c1cc(F)ccc1Oc1nncnc1N1CC2(CCN(Cc3ccc(NS(C)(=O)=O)cc3C)CC2)C1)C(C)C] | 626.3 | ¹H NMR (400 MHz, CD₃OD) δ 8.88 (d, J = 9.5 Hz, 1H), 7.58-7.47 (m, 2H), 7.41-7.26 (m, 2H), 7.23-7.16 (m, 2H), 4.80 (s, 1H), 4.64 (s, 1H), 4.44 (s, 1H), 4.38-4.35 (m, 2H), 4.28 (s, 1H), 3.88-3.82 (m, 1H), 3.58-3.50 (m, 2H), 3.45-3.36 (m, 1H), 3.28-3.10 (m, 3H), 3.00 (s, 3H), 2.46 (d, J = 8.0 Hz, 3H), 2.37- 2.27 (m, 2H), 2.25- 2.09 (m, 2H), 1.35- 1.26 (m, 3H), 1.20 (t, J = 6.8 Hz, 3H), 1.16- 0.99 (m, 3H). | 10-2 |
| 24 | 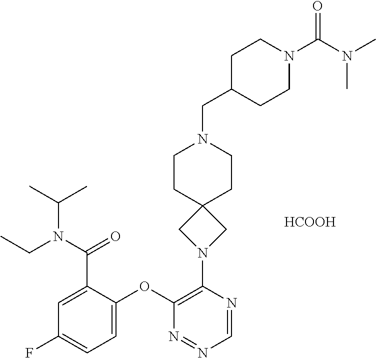 [SMILES: CCN(C(=O)c1cc(F)ccc1Oc1nncnc1N1CC2(CCN(CC3CCN(C(=O)N(C)C)CC3)CC2)C1)C(C)C ; "HCOOH"] | 597.6 | ¹H NMR (400 MHz, CD₃OD) δ 8.42 (s, 1H), 8.34 (s, 1H), 7.42-7.38 (m, 1H), 7.34-7.17 (m, 2H), 4.46 (s, 2H), 4.05- 4.02 (m, 2H), 3.86- 3.76 (m, 1H), 3.70- 3.66 (m, 2H), 3.49- 3.46 (m, 1H), 3.30- 3.12 (m, 5H), 2.96- 2.93 (m, 2H), 2.88- 2.74 (m, 8H), 2.17- 2.14 (m, 4H), 2.06- 2.02 (m, 1H), 1.81- 1.77 (m, 2H), 1.21- 1.15 (m, 9H), 0.83- 0.78 (m, 2H). | 10-1 |
| 25 | 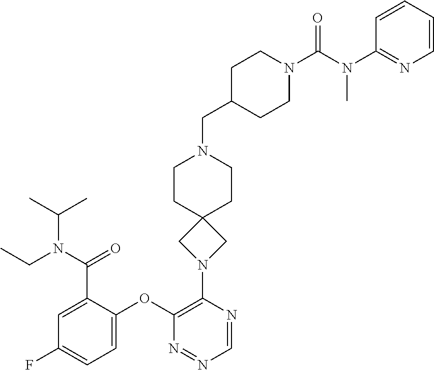 [SMILES: CCN(C(=O)c1cc(F)ccc1Oc1nncnc1N1CC2(CCN(CC3CCN(C(=O)N(C)c4ccccn4)CC3)CC2)C1)C(C)C] | 660.4 | ¹H NMR (400 MHz, CD₃OD) δ 8.37 (s, 1H), 8.27 (d, J = 4.0 Hz, 1H), 7.75-7.67 (m, 1H), 7.42-7.37 (m, 1H), 7.31-7.16 (m, 2H), 7.01-6.98 (m, 2H), 4.40-4.30 (m, 2H), 3.95-3.83 (m, 4H), 3.83-3.77 (m, 1H), 3.55-3.42 (m, 1H), 3.24 (s, 3H), 3.23-3.16 (m, 1H), 2.79 (t, J = 11.9 Hz, 2H), 2.58-2.21 (br, 4H), 2.16 (d, J = 6.6 Hz, 2H), 1.88-1.81 (m, 4H), 1.71 (d, J = 11.6 Hz, 3H), 1.20 (d, J = 6.7 Hz, 2H), 1.17-1.10 (m, 5H), 1.09-1.02 (m, 2H), 0.80 (d, J = 6.1 Hz, 2H). | 10-1 |
| 180 | 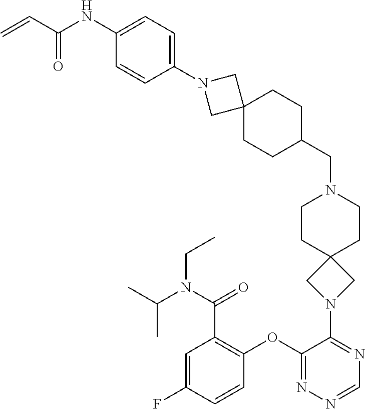 [SMILES: C=CC(=O)Nc1ccc(N2CC3(CCC(CN4CCC5(CC4)CN(c4ncnnc4Oc4ccc(F)cc4C(=O)N(CC)C(C)C)C5)CC3)C2)cc1] | 711.4 | ¹H NMR (400 MHz, DMSO-d₆) δ 9.86 (s, 1H), 8.45 (s, 1H), 7.46-7.43 (m, 3H), 7.36-7.33 (m, 2H), 6.38-6.35 (m, 3H), 6.20-6.14 (m, 1H), 5.67 (d, J = 12.0 Hz, 1H), 4.20 (s, 2H), 3.79 (s, 2H), 3.63- 3.59 (m, 1H), 3.47 (s, 3H), 3.42 (s, 3H), 2.38- 2.31 (m, 4H), 2.03 (d, J = 6.8 Hz, 2H), 1.86- 1.83 (m, 2H), 1.73 (s, 4H), 1.66 (d, J = 11.8 Hz, 2H), 1.45 (t, J = 11.3 Hz, 4H), 1.12-1.01 (m, 6H), 0.90 (d, J = 13.7 Hz, 2H), 0.71 (d, J = 6.1 Hz, 2H). | 10-7 |
| 181 | 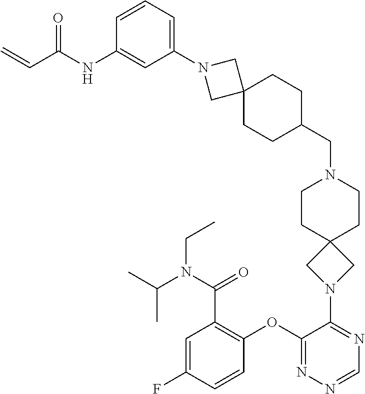 [SMILES: C=CC(=O)Nc1cccc(N2CC3(CCC(CN4CCC5(CC4)CN(c4ncnnc4Oc4ccc(F)cc4C(=O)N(CC)C(C)C)C5)CC3)C2)c1] | 711.4 | ¹H NMR (400 MHz, CD₃OD) δ 8.37 (d, J = 0.7 Hz, 1H), 7.40 (dt, J = 9.5, 4.8 Hz, 1H), 7.27 (td, J = 8.5, 3.3 Hz, 1H), 7.19 (dd, J = 8.0, 2.8 Hz, 1H), 7.10 (t, J = 8.0 Hz, 1H), 6.89-6.84 (m, 2H), 6.40 (dd, J = 8.8, 8.2 Hz, 1H), 6.31 (dd, J = 16.9, 1.5 Hz, 1H), 6.23 (d, J = 8.1 Hz, 1H), 5.73 (dd, J = 9.8, 1.5 Hz, 1H), 4.60 (s, 2H), 4.35 (d, J = 14.2 Hz, 2H), 3.91 (d, J = 7.5 Hz, 2H), 3.80 (dt, J = 13.3, 6.6 Hz, 1H), 3.56 (s, 2H), 3.49 (s, 2H), 3.33 (d, J = 0.8 Hz, 1H), 2.43 (s, 4H), 2.17 (d, J = 7.1 Hz, 2H), 1.95 (d, J = 12.9 | 10-8 |
| | | | Hz, 2H), 1.87 (s, 4H), | |
| | | | 1.76 (d, J = 11.3 Hz, | |
| | | | 2H), 1.53 (dd, J = | |
| | | | 12.6, 10.0 Hz, 4H), 1.20 | |
| | | | (d, J = 6.8 Hz, 1H), 1.14 | |
| | | | (t, J = 6.8 Hz, 4H), 1.07- | |
| | | | 0.98 (m, 3H), 0.80 (d, | |
| | | | J = 6.2 Hz, 2H). | |
| 182 | 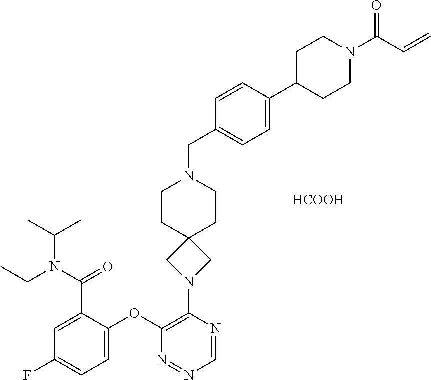 [SMILES: C=CC(=O)N1CCC(c2ccc(CN3CCC4(CC3)CN(c3ncnnc3Oc3ccc(F)cc3C(=O)N(CC)C(C)C)C4)cc2)CC1 ; "HCOOH"] | 656.4 | 1H NMR (400 MHz, CD₃OD) δ 8.40 (s, 1H), 8.36 (s, 1H), 7.43-7.31 (m, 5H), 7.31-7.19 (m, 2H), 6.80 (dd, J = 16.8, 10.7 Hz, 1H), 6.20 (dd, J = 16.8, 2.0 Hz, 1H), 5.74 (dd, J = 10.7, 2.0 Hz, 1H), 4.75-4.67 (m, 1H), 4.49-4.36 (m, 2H), 4.29-4.20 (m, 1H), 4.10 (s, 2H), 4.06-3.96 (m, 2H), 3.85-3.74 (m, 1H), 3.52-3.39 (m, 1H), 3.27-3.17 (m, 2H), 3.15- 2.96 (m, 4H), 2.94-2.75 (m, 2H), 2.11-2.01 (m, 4H), 1.92 (s, 2H), 1.91- 1.85 (m, 1H), 1.70-1.54 (m, 2H), 1.2-1.269 (m, 1H), 1.19-1.16 (m, 2H), 1.14-1.09 (m, 3H), 0.78 (d, J = 6.1 Hz, 2H). | 10-5 |
| 183 | 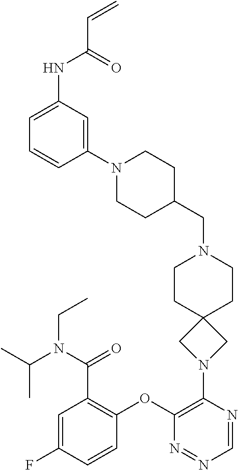 [SMILES: C=CC(=O)Nc1cccc(N2CCC(CN3CCC4(CC3)CN(c3ncnnc3Oc3ccc(F)cc3C(=O)N(CC)C(C)C)C4)CC2)c1] | 671.4 | ¹H NMR (400 MHz, DMSO-d₆) δ 9.94 (s, 1H), 8.42 (s, 1H), 8.12 (s, 1H), 7.44-7.38 (m, 1H), 7.35-7.30 (m, 2H), 7.26 (s, 1H), 7.11-6.99 (m, 2H), 6.61 (d, J = 8.1 Hz, 1H), 6.38 (dd, J = 17.0, 10.1 Hz, 1H), 6.19 (dd, J = 16.9, 2.0 Hz, 1H), 5.69 (dd, J = 10.0, 2.0 Hz, 1H), 4.22 (d, J = 21.4 Hz, 3H), 3.78 (s, 4H), 2.61 (t, J = 11.4 Hz, 3H), 2.29 (s, 2H), 2.12 (d, J = 6.9 Hz, 2H), 1.68 (d, J = 29.8 Hz, 8H), 1.23-0.86 (m, 11H), 0.68 (d, J = 6.3 Hz, 2H). | 10-6 |

### Compound 26: N-cyclopropyl-4-((2-(6-(2-(ethyl(isopropyl)carbamoyl)-4-fluorophenoxy)-1,2,4-triazin-5-yl)-2,7-diazaspiro[3.5]nonan-7-yl)methyl)piperidine-1-carboxamide

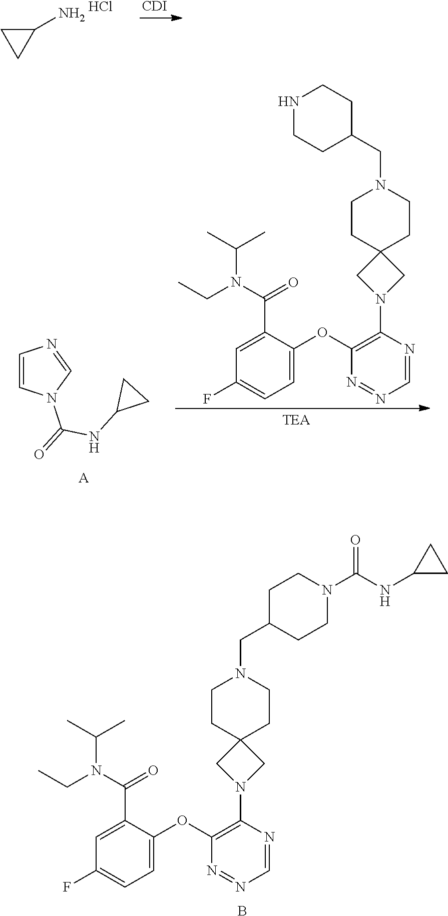

### (A) N-cyclopropyl-1H-imidazole-1-carboxamide

To a reaction flask were added cyclopropylamine (693 mg, 7.41 mmol), N,N′-carbonyldiimidazole (1.15 g, 8.16 mmol) and dichloromethane (30 ml), and the mixture was stirred overnight at room temperature. The reaction solution was concentrated, and the residue was purified with flash column chromatography (eluted with a gradient of dichloromethane/methanol=100:0-0:100) to obtain 907 mg of white solid product. MS (m/z): 152.1 [M+H]⁺.

### (B) N-cyclopropyl-4-((2-(6-(2-(ethyl(isopropyl)carbamoyl)-4-fluorophenoxy)-1,2,4-triazin-5-yl)-2,7-diazaspiro[3.5]nonan-7-yl)methyl)piperidine-1-carboxamide

To a reaction flask were added N-cyclopropyl-1H-imidazole-1-carboxamide (40 mg, 0.27 mmol), intermediate 10-1 (70 mg, 0.133 mmol), triethylamine (27 mg, 0.27 mmol) and dichloromethane (10 ml), and the mixture was stirred overnight at room temperature.

The reaction solution was then concentrated, and the residue was purified with flash column chromatography (eluted with a gradient of water/methanol=100:0-0:100) to obtain 50 mg of white solid product. MS (m/z): 609.4 [M+H]⁺.

¹H NMR (400 MHz, CD₃OD) δ 8.38 (s, 1H), 8.34 (s, 1H), 7.38-7.35 (m, 1H), 7.28-7.20 (m, 1H), 7.18-7.12 (m, 1H), 4.45-4.16 (m, 2H), 3.95 (d, J=13.2 Hz, 4H), 3.87-3.75 (m, 1H), 3.52-3.33 (m, 1H), 3.18-3.08 (m, 1H), 2.90-2.69 (m, 6H), 2.60-2.47 (m, 3H), 2.03-1.97 (m, 4H), 1.92-1.82 (m, 1H), 1.75 (d, J=13.1 Hz, 2H), 1.25-1.01 (m, 9H), 0.95-0.75 (m, 2H), 0.67-0.59 (m, 2H), 0.46-0.39 (m, 2H).

The following compounds were prepared according to the preparation procedure of compound 26 using corresponding intermediates and reagents under appropriate conditions that will be recognized by those skilled in the art.

| | | MS | | Intermediate |
|---|---|---|---|---|
| Compound | Structural formula | (M + H)⁺ | ¹HNMR | used |
| 27 | 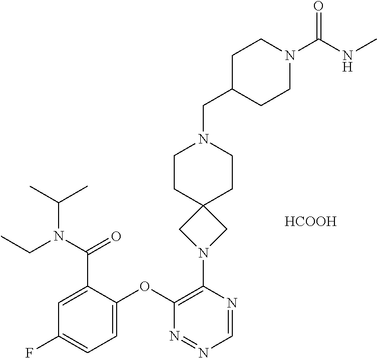 [SMILES: CCN(C(=O)c1cc(F)ccc1Oc1nncnc1N1CC2(CCN(CC3CCN(C(=O)NC)CC3)CC2)C1)C(C)C ; "HCOOH"] | 583.6 | ¹H NMR (400 MHz, CD₃OD) δ 8.41 (s, 2H), 7.42-7.39 (m, 1H), 7.34-7.18 (m, 2H), 4.44 (s, 2H), 4.01 (d, J = 10.7 Hz, 4H), 3.89-3.75 (m, 1H), 3.55- 3.42 (m, 1H), 3.27-3.17 (m, 1H), 3.16-2.95 (m, 4H), 2.85- 2.75 (m, 4H), 2.70 (s, 3H), 2.14-2.05 (m, 4H), 2.04-1.95 (m, 1H), 1.77 (d, J = 12.3 Hz, 2H), 1.23-1.12 (m, 8H), 1.07 (t, J = 7.1 Hz, 1H), 0.80 (d, J = 5.0 Hz, 2H). | 10-1 |
| 28 | 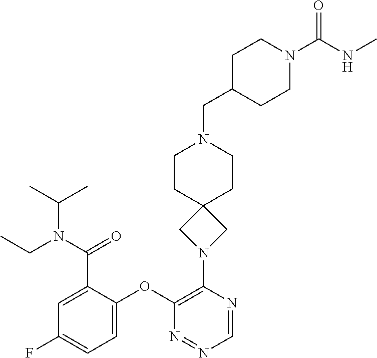 [SMILES: CCN(C(=O)c1cc(F)ccc1Oc1nncnc1N1CC2(CCN(CC3CCN(C(=O)NC)CC3)CC2)C1)C(C)C] | 597.4 | ¹H NMR (400 MHz, CD₃OD) δ 8.38 (s, 1H), 8.34 (s, 1H), 7.38-7.35 (m, 1H), 7.29-7.20 (m, 1H), 7.15 (d, J = 7.8 Hz, 1H), 4.45-4.20 (m, 2H), 4.05- 3.95 (m, 4H), 3.88-3.76 (m, 1H), 3.46-3.33 (m, 1H), 3.16 (q, J = 7.2 Hz, 3H), 2.90- 2.72 (m, 6H), 2.57 (d, J = 6.9 Hz, 2H), 2.00 (t, J = 5.5 Hz, 4H), 1.94-1.82 (m, 1H), 1.76 (d, J = 12.6 Hz, 2H), 1.22- 1.18 (m, 2H), 1.18-1.10 (m, 5H), 1.11-1.05 (m, 4H), 1.04- 1.01 (m, 1H), 0.850.77 (m, 2H). | 10-1 |
| 29 | 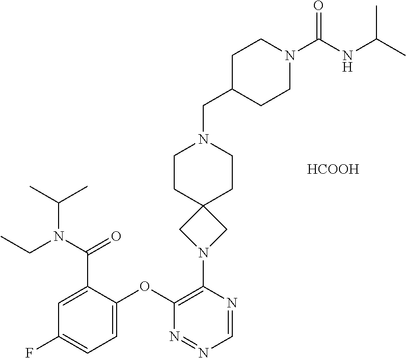 [SMILES: CCN(C(=O)c1cc(F)ccc1Oc1nncnc1N1CC2(CCN(CC3CCN(C(=O)NC(C)C)CC3)CC2)C1)C(C)C ; "HCOOH"] | 611.4 | ¹H NMR (400 MHz, CD₃OD) δ 8.38 (s, 2H), 7.39-7.35 (m, 1H), 7.29-7.20 (m, 1H), 7.18- 7.12 (m, 1H), 4.46-4.20 (m, 2H), 4.06-3.95 (m, 4H), 3.92- 3.74 (m, 2H), 3.45-3.36 (m, 1H), 3.18-3.09 (m, 1H), 2.88- 2.71 (m, 6H), 2.56 (d, J = 6.9 Hz, 2H), 2.02-1.97(m, 4H), 1.94-1.81 (m, 1H), 1.78- 1.71 (m, 2H), 1.23-1.17 (m, 2H), 1.16-1.09 (m, 11H), 1.08- 1.02 (m, 2H), 0.91-0.78 (m, 2H). | 10-1 |
| 30 | 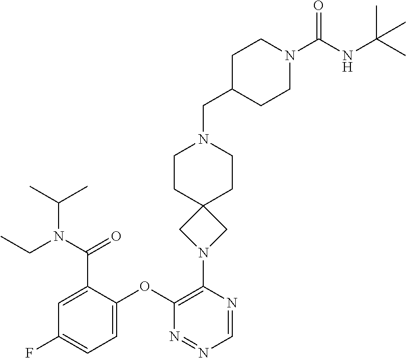 [SMILES: CCN(C(=O)c1cc(F)ccc1Oc1nncnc1N1CC2(CCN(CC3CCN(C(=O)NC(C)(C)C)CC3)CC2)C1)C(C)C] | 625.6 | ¹H NMR (400 MHz, CD₃OD) δ 8.37 (s, 1H), 7.42-7.37 (m, 1H), 7.31-7.23 (m, 1H), 7.23- 7.16 (m, 1H), 4.45-4.24 (m, 2H), 3.99-3.86 (m, 4H), 3.84- 3.77 (m, 1H), 3.54-3.42 (m, 1H), 3.27-3.12 (m, 1H), 2.70 (t, J = 12.0 Hz, 2H), 2.52- 2.25 (br, 4H), 2.17 (d, J = 6.4 Hz, 2H), 1.88-1.82 (m, 4H), 1.77-1.68 (m, 3H), 1.30 (s, 9H), 1.20 (d, J = 6.8 Hz, 2H), 1.16-1.09 (m, 5H), 1.07-1.02 (m, 2H), 0.80 (d, J = 5.8 Hz, 2H). | 10-1 |
| 31 | 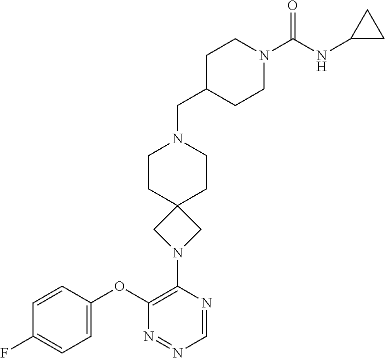 [SMILES: O=C(NC1CC1)N1CCC(CN2CCC3(CC2)CN(c2ncnnc2Oc2ccc(F)cc2)C3)CC1] | 496.4 | ¹H NMR (400 MHz, CD₃OD) δ 8.34 (s, 1H), 7.31-7.20 (m, 2H), 7.20-7.08 (m, 2H), 4.39 (s, 2H), 4.02-3.88 (m, 4H), 2.74-2.70 (m, 2H), 2.52-2.48 (m, 1H), 2.46-2.40 (m, 4H), 2.19-2.16 (m, 2H), 1.89-1.86 (m, 4H), 1.75-1.70 (m, 3H), 1.06-1.02 (m, 2H), 0.67-0.55 (m, 2H), 0.47-0.36 (m, 2H). | 10-4 |
| 32 | 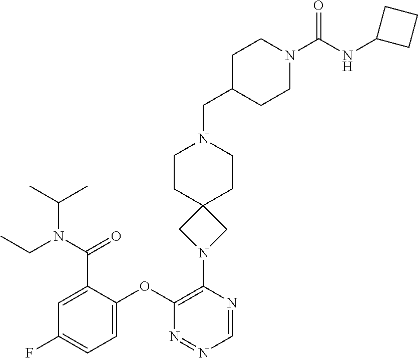 [SMILES: CCN(C(=O)c1cc(F)ccc1Oc1nncnc1N1CC2(CCN(CC3CCN(C(=O)NC4CCC4)CC3)CC2)C1)C(C)C] | 623.4 | ¹H NMR (400 MHz, CD₃OD) δ 8.36 (s, 1H), 7.41-7.37 (m, 1H), 7.30-7.22 (m, 1H), 7.20- 7.17 (m, 1H), 4.40-4.30 (m, 2H), 4.23-4.12 (m, 1H), 3.99 (d, J = 13.1 Hz, 2H), 3.93-3.86 (m, 2H), 3.83-3.78(m, 1H), 3.55-3.46 (m, 1H), 3.27-3.12 (m, 1H), 2.73 (t, J = 12.0 Hz, 2H), 2.53-2.25 (m, 4H), 2.24- 2.19 (m, 2H), 2.17 (d, J = 6.5 Hz, 2H), 1.98-1.89 (m, 2H), 1.88-1.83 (m, 4H), 1.77-1.71 (m, 3H), 1.67-1.60 (m, 2H), 1.20 (d, J = 6.8 Hz, 2H), 1.16- 1.11 (m, 4H), 1.08-0.99 (m, 3H), 0.80 (d, J = 5.2 Hz, 2H). | 10-1 |
| 33 | 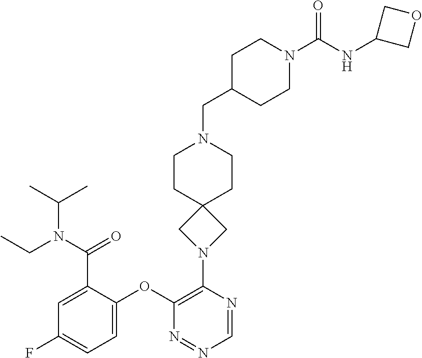 [SMILES: CCN(C(=O)c1cc(F)ccc1Oc1nncnc1N1CC2(CCN(CC3CCN(C(=O)NC4COC4)CC3)CC2)C1)C(C)C] | 625.4 | ¹H NMR (400 MHz, CD₃OD) δ 8.47 (s, 1H), 8.41 (s, 1H), 7.42-7.39 (m, 1H), 7.32-7.19 (m, 2H), 4.83-4.77 (m, 3H), 4.59-4.55 (m, 2H), 4.45-4.36 (m, 2H), 4.05 (d, J = 13.4 Hz, 2H), 4.02-3.93 (m, 2H), 3.87- 3.77 (m, 1H), 3.55-3.45 (br, 1H), 3.27-3.18 (br, 1H), 3.00- 2.85 (br, 4H), 2.84-2.78 (m, 2H), 2.64 (d, J = 6.7 Hz, 2H), 2.06-2.01 (m, 3H), 2.00-1.90 (m, 2H), 1.78 (d, J = 12.1 Hz, 2H), 1.23-1.03 (m, 9H), 0.80 (d, J = 5.5 Hz, 2H). | 10-1 |
| 34 | 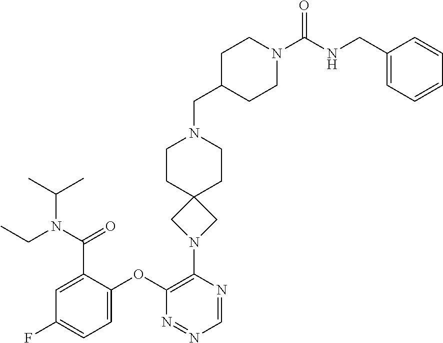 [SMILES: CCN(C(=O)c1cc(F)ccc1Oc1nncnc1N1CC2(CCN(CC3CCN(C(=O)NCc4ccccc4)CC3)CC2)C1)C(C)C] | 659.4 | ¹H NMR (400 MHz, DMSO-d₆) δ 8.43 (d, J = 0.8 Hz, 1H), 7.42 (dd, J = 6.6, 3.6 Hz, 1H), 7.36- 7.32 (m, 2H), 7.26 (d, J = 7.7 Hz, 2H), 7.18 (dd, J = 17.2, 7.3 Hz, 3H), 6.99 (t, J = 5.8 Hz, 1H), 4.20 (d, J = 2.9 Hz, 2H), 4.19 (s, 2H), 3.94 (d, J = 13.0 Hz, 2H), 3.79 (d, J = 7.9 Hz, 2H), 3.58 (dd, J = 13.3, 6.6 Hz, 1H), 3.07-3.04 (m, 1H), 2.61 (d, J = 12.0 Hz, 2H), 2.31 (s, 4H), 2.05 (d, J = 6.5 Hz, 2H), 1.72-1.59 (m, 8H), 1.07-0.98 (m, 6H), 0.91 (t, J = 7.1 Hz, 3H), 0.69 (d, J = 6.3 Hz, 2H). | 10-1 |
| 35 | 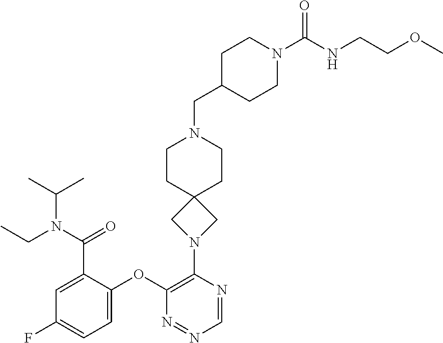 [SMILES: CCN(C(=O)c1cc(F)ccc1Oc1nncnc1N1CC2(CCN(CC3CCN(C(=O)NCCOC)CC3)CC2)C1)C(C)C] | 627.4 | ¹H NMR (400 MHz, DMSO-d₆) δ 8.45 (d, J = 3.4 Hz, 1H), 7.47- 7.41 (m, 1H), 7.39-7.34 (m, 2H), 6.45 (t, J = 5.0 Hz, 1H), 4.21 (s, 2H), 3.90 (d, J = 12.9 Hz, 2H), 3.81 (d, J = 8.2 Hz, 2H), 3.30 (t, J = 5.5 Hz, 2H), 3.22 (s, 3H), 3.14 (d, J = 5.8 Hz, 2H), 3.10- 2.94 (m, 2H), 2.58 (d, J = 12.2 Hz, 2H), 2.33 (s, 4H), 2.06 (d, J = 6.5 Hz, 2H), 1.74-1.59 (m, 8H), 1.06-0.93 (m, 6H), 0.87 (d, J = 11.4 Hz, 3H), 0.71 (s, 2H). | 10-1 |

### Compound 36: 4-((2-(6-(2-(ethyl(isopropyl)carbamoyl)-4-fluorophenoxy)-1,2,4-triazin-5-yl)-2,7-diazaspiro[3.5]nonan-7-yl)methyl)-N-(2-fluorophenyl)piperidine-1-carboxamide

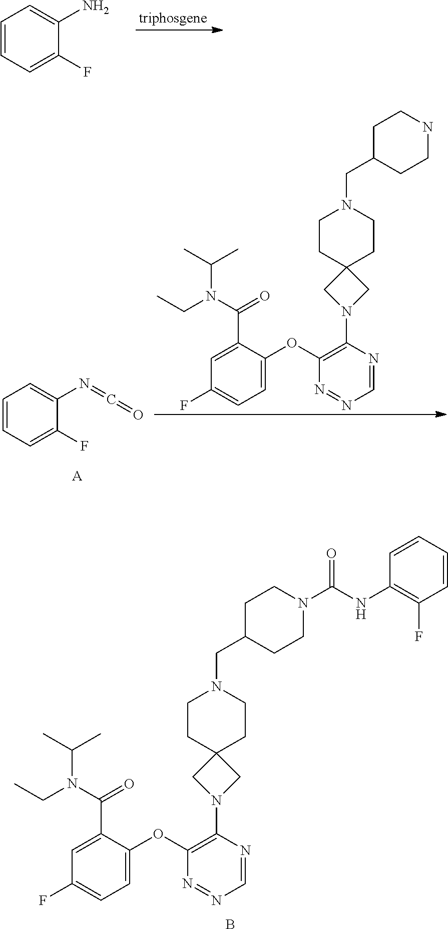

### (A) 1-fluoro-2-isocyanatobenzene

To a reaction flask were added 2-fluoroaniline (200 mg, 1.78 mmol), pyridine (570 mg, 7.21 mmol) and dichloromethane (20 ml), triphosgene (540 mg, 1.82 mmol) was added dropwise under nitrogen protection, and the mixture was stirred and reacted for 15 hours at room temperature. After the reaction was completed, the reaction solution was washed with 1 mole/liter dilute hydrochloric acid and a saturated aqueous sodium chloride solution, and the organic phase was concentrated to obtain a crude product, which was directly used in the next reaction. MS (m/z): 241.1 [2M+H]⁺.

### (B) 4-((2-(6-(2-(ethyl(isopropyl)carbamoyl)-4-fluorophenoxy)-1,2,4-triazin-5-yl)-2,7-diazaspiro[3.5]nonan-7-yl)methyl)-N-(2-fluorophenyl)piperidine-1-carboxamide

To a sealed tube were added the crude product 1-fluoro-2-isocyanatobenzene (150 mg) obtained in the previous step, intermediate 10-1 (50 mg, 0.095 mmol), triethylamine (19 mg, 0.19 mmol) and dichloromethane (20 ml), and then the mixture was heated to 80° C., stirred and reacted for 15 hours. After the reaction was completed, the reaction solution was concentrated, and the residue was purified with flash column chromatography (eluted with a gradient of water/methanol=100:0-0:100) to obtain 30 mg of white solid. MS (m/z): 663.4 [M+H]⁺.

¹H NMR (400 MHz, CD₃OD) δ 8.35 (s, 1H), 8.18 (d, J=4.8 Hz, 1H), 7.76-7.73 (m, 1H), 7.69-7.65 (m, 1H), 7.41-7.34 (m, 1H), 7.26-7.22 (m, 1H), 7.17-7.10 (m, 1H), 7.02-6.92 (m, 1H), 4.40-4.20 (m, 2H), 4.13 (d, J=13.2 Hz, 2H), 4.05-3.74 (m, 3H), 3.55-3.33 (m, 1H), 3.19-3.06 (m, 1H), 2.93 (t, J=12.2 Hz, 2H), 2.47-2.33 (br, 4H), 2.21 (d, J=6.4 Hz, 2H), 1.89-1.86 (m, 5H), 1.85-1.77 (m, 2H), 1.24-1.17 (m, 3H), 1.16-1.12 (m, 4H), 1.07-1.01 (m, 2H), 0.91-0.72 (m, 2H).

The following compounds were prepared according to the preparation procedure of compound 36 using corresponding intermediates and reagents under appropriate conditions that will be recognized by those skilled in the art.

| | | MS | | Intermediate |
|---|---|---|---|---|
| Compound | Structural formula | (M + H)⁺ | ¹HNMR | used |
| 37 | 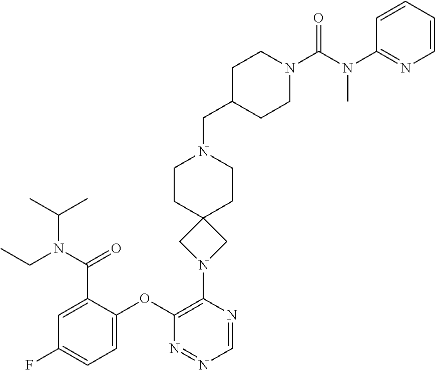 [SMILES: CCN(C(=O)c1cc(F)ccc1Oc1nncnc1N1CC2(CCN(CC3CCN(C(=O)N(C)c4ccccn4)CC3)CC2)C1)C(C)C] | 646.4 | ¹H NMR (400 MHz, CD₃OD) δ 8.35 (s, 1H), 8.18 (d, J = 4.8 Hz, 1H), 7.75 (d, J = 8.4 Hz, 1H), 7.67 (t, J = 7.0 Hz, 1H), 7.41- 7.34 (m, 1H), 7.23 (t, J = 8.4 Hz, 1H), 7.13 (d, J = 7.7 Hz, 1H), 7.02-6.92 (m, 1H), 4.40- 4.20 (m, 2H), 4.13 (d, J = 13.2 Hz, 2H), 4.05-3.74 (m, 3H), 3.55-3.33 (m, 1H), 3.19-3.06 (m, 1H), 2.93 (t, J = 12.2 Hz, 2H), 2.47-2.33 (br, 4H), 2.21 (d, J = 6.4 Hz, 2H), 1.89-1.86 (m, 5H), 1.85-1.77 (m, 2H), 1.24-1.17 (m, 3H), 1.16-1.12 (m, 4H), 1.07-1.01 (m, 2H), 0.91-0.72 (m, 2H). | 10-1 |

### Compound 38: N-ethyl-5-fluoro-N-isopropyl-2-((5-(7-((1-(6-(trifluoromethyl)pyridazin-3-yl)piperidin-4-yl)methyl)-2,7-diazaspiro[3.5]nonan-2-yl)-1,2,4-triazin-6-yl)oxy)benzamide

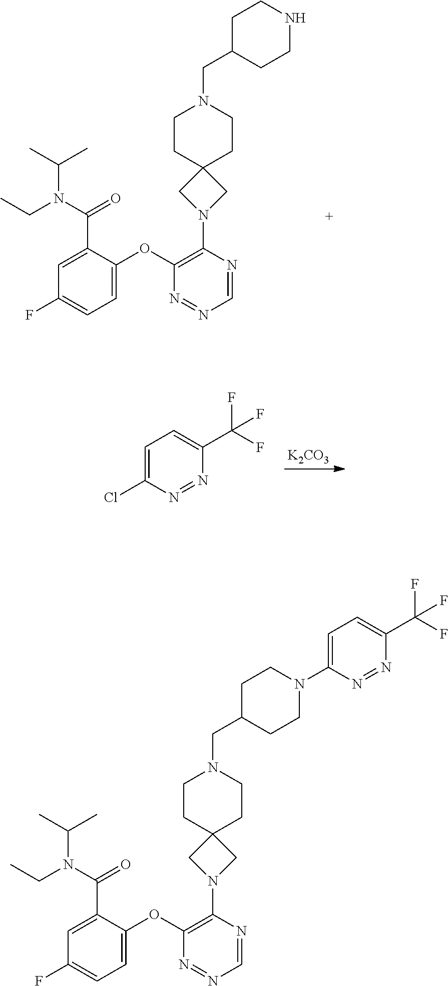

To a reaction flask were added successively intermediate 10-1 (70 mg, 0.13 mmol), 3-chloro-6-(trifluoromethyl)pyridazine (29 mg, 0.16 mmol), potassium carbonate (46 mg, 0.33 mmol) and N,N-dimethylformamide (5 ml), and the mixture was heated to 120° C. and reacted for 3 hours. After the reaction was completed, the reaction solution was cooled to room temperature, and purified with flash column chromatography (eluted with a gradient of water/methanol=100:0-0:100) to obtain 35 mg of white solid. MS (m/z): 672.4 [M+H]⁺.

¹H NMR (400 MHz, CD₃OD) δ 8.45 (s, 1H), 8.40 (s, 1H), 7.64 (d, J=9.7 Hz, 1H), 7.42-7.38 (m, 1H), 7.33 (d, J=9.8 Hz, 1H), 7.30-7.19 (m, 2H), 4.55 (d, J=13.5 Hz, 2H), 4.44-4.38 (m, 2H), 4.02-3.96 (m, 2H), 3.86-3.74 (m, 3H), 3.54-3.41 (m, 1H), 3.27-3.17 (m, 1H), 3.08 (t, J=12.1 Hz, 2H), 3.03-2.70 (m, 4H), 2.63 (d, J=6.6 Hz, 2H), 2.14-2.07 (m, 1H), 2.06-1.98 (m, 4H), 1.91 (d, J=12.8 Hz, 2H), 1.35-1.25 (m, 2H), 1.20-1.12 (m, 6H), 1.06 (t, J=7.1 Hz, 1H), 0.79 (d, J=6.2 Hz, 2H).

The following compounds were prepared according to the preparation procedure of compound 38 using corresponding intermediates and reagents under appropriate conditions that will be recognized by those skilled in the art.

| | | MS | | Intermediate |
|---|---|---|---|---|
| Compound | Structural formula | (M + H)⁺ | ¹HNMR | used |
| 39 | 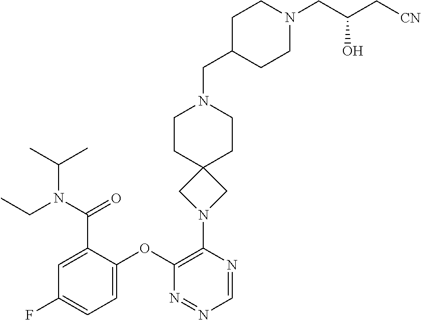 [SMILES: CCN(C(=O)c1cc(F)ccc1Oc1nncnc1N1CC2(CCN(CC3CCN(C[C@@H](O)CC#N)CC3)CC2)C1)C(C)C] | 609.4 | ¹H NMR (400 MHz, DMSO-d₆) δ 8.44 (t, J = 8.0 Hz, 1H), 7.41 (d, J = 4.2 Hz, 1H), 7.33 (dd, J = 7.0, 3.1 Hz, 2H), 4.18 (s, 2H), 3.87-3.75 (m, 3H), 3.64-3.54 (m, 1H), 2.96 (s, 1H), 2.75 (d, J = 12.2 Hz, 2H), 2.65-2.59 (m, 1H), 2.28-2.22 (m, 6H), 2.03 (d, J = 7.1 Hz, 2H), 2.00- 1.83 (m, 4H), 1.73- 1.55 (m, 8H), 1.10- 0.99 (m, 9H), 0.69 (d, J = 6.2 Hz, 2H). | 10-1 |
| 40 | 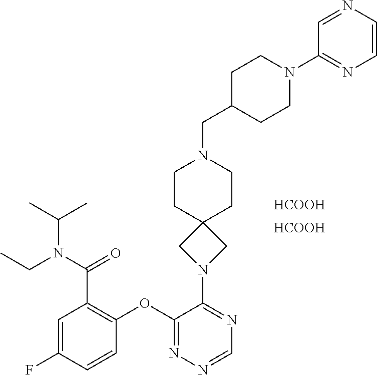 [SMILES: CCN(C(=O)c1cc(F)ccc1Oc1nncnc1N1CC2(CCN(CC3CCN(c4cnccn4)CC3)CC2)C1)C(C)C ; "HCOOH" ; "HCOOH"] | 604.6 | ¹H NMR (400 MHz, CD₃OD) δ 8.42 (s, 1H), 8.29 (s, 2H), 8.19 (s, 1H), 8.07 (s, 1H), 7.73 (d, J = 2.0 Hz, 1H), 7.39 (dt, J = 9.0, 4.5 Hz, 1H), 7.33-7.19 (m, 2H), 4.51-4.38 (m, 4H), 4.10-3.96 (m, 2H), 3.87-3.75 (m, 1H), 3.53- 3.42 m, 1H), 3.27-3.10 (m, 4H), 3.00-2.91 (m, 4H), 2.23-2.08 (m, 5H), 1.93-1.80 (m, 2H), 1.38-1.22 (m, 3H), 1.24-1.03 (m, 7H), 0.79 (d, J = 6.0 Hz, 2H). | 10-1 |
| 41 | 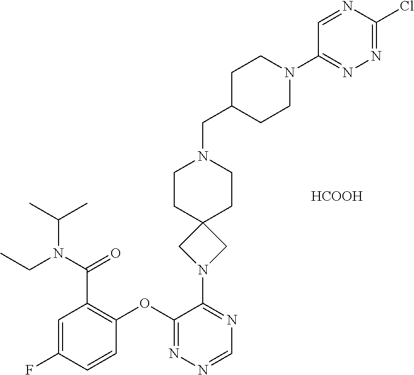 [SMILES: CCN(C(=O)c1cc(F)ccc1Oc1nncnc1N1CC2(CCN(CC3CCN(c4cnc(Cl)nn4)CC3)CC2)C1)C(C)C ; "HCOOH"] | 639.6 | ¹H NMR (400 MHz, CD₃OD) δ 8.42 (s, 1H), 8.34 (s, 1H), 8.30 (s, 1H), 7.34-7.36(m, 1H), 7.33-7.20 (m, 2H), 4.76 (d, J = 13.4 Hz, 2H), 4.45 (s, 2H), 4.10-3.95 (m, 2H), 3.86-3.75 (m, 1H), 3.52-3.37 (m, 1H), 3.27- 3.11 (m, 4H), 3.10-3.02 (m, 2H), 2.92-2.87 (m, 2H), 2.24-2.15 (m, 1H), 2.14-2.05 (m, 4H), 1.94- 1.83 (m, 2H), 1.36-1.23 (m, 3H), 1.21-1.10 (m, 6H), 1.07 (t, J = 7.1 Hz, 1H), 0.79 (d, J = 6.4 Hz, 2H). | 10-1 |
| 42 | 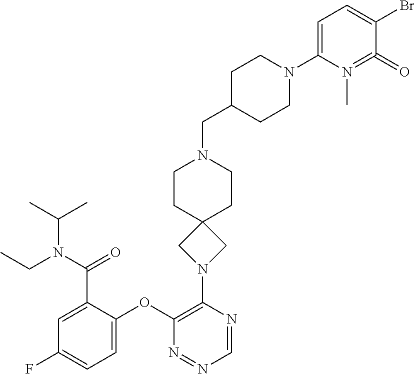 | 711.3 | ¹H NMR (400 MHz, CD₃OD) δ 8.38 (s, 1H), 7.80 (d, J = 8.1 Hz, 1H), 7.43-7.38 (m, 1H), 7.32-7.18 (m, 2H), 5.96 (d, J = 8.2 Hz, 1H), 4.43- 4.22 (m, 2H), 3.98- 3.87 (m, 2H), 3.85- 3.75 (m, 1H), 3.59 (s, 3H), 3.55-3.45 (m, 1H), 3.27-3.18 (m, 3H), 2.69 (t, J = 11.3 Hz, 2H), 2.59-2.32 (m, 4H), 2.26 (d, J = 7.0 Hz, 2H), 1.95- 1.90 (m, 1H), 1.88-1.84 (m, 5H), 1.80-1.71 (m, 1H), 1.44-1.35 (m, 2H), 1.22-1.13 (m, 6H), 1.06 (t, J = 7.1 Hz, 1H), 0.81 (d, J = 6.3 Hz, 2H). | 10-1 |
| 43 | 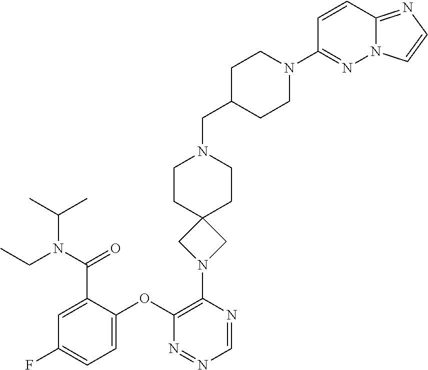 [SMILES: CCN(C(=O)c1cc(F)ccc1Oc1nncnc1N1CC2(CCN(CC3CCN(c4ccc5nccn5n4)CC3)CC2)C1)C(C)C] | 643.6 | ¹H NMR (400 MHz, CD₃OD) δ 8.37 (s, 1H), 7.73 (s, 1H), 7.66 (d, J = 10.0 Hz, 1H), 7.43 (d, J = 1.1 Hz, 1H), 7.42-7.38 (m, 1H), 7.31- 7.24 (m, 1H), 7.25-7.18 (m, 1H), 7.13 (d, J = 10.1 Hz, 1H), 4.41- 4.33 (m, 2H), 4.20 (d, J = 13.1 Hz, 2H), 3.97- 3.88 (m, 2H), 3.86-3.76 (m, 1H), 3.57-3.42 (m, 1H), 3.28-3.16 (m, 1H), 2.91 (t, J = 11.9 Hz, 2H), 2.58-2.30 (br, 4H), 2.21 (d, J = 6.4 Hz, 2H), 1.90-1.86 (m, 5H), 1.85-1.82 (m, 2H), 1.26-1.19 (m, 3H), 1.17-1.10 (m, 5H), 1.05 (t, J = 7.1 Hz, 1H), 0.80 (d, J = 6.4 Hz, 2H). | 10-1 |
| 44 | 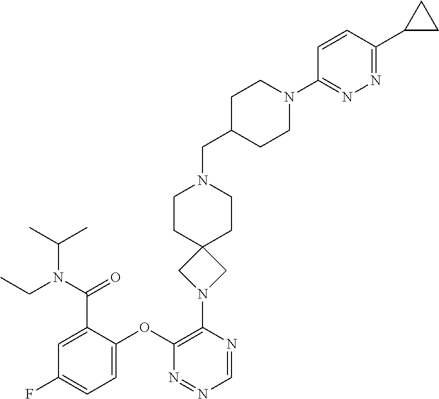 [SMILES: CCN(C(=O)c1cc(F)ccc1Oc1nncnc1N1CC2(CCN(CC3CCN(c4ccc(C5CC5)nn4)CC3)CC2)C1)C(C)C] | 644.4 | ¹H NMR (400 MHz, CD₃OD) δ 8.29 (s, 1H), 7.32 (dd, J = 9.0, 4.5 Hz, 1H), 7.19 (dt, J = 11.8, 5.8 Hz, 1H), 7.11 (dd, J = 8.0, 2.7 Hz, 1H), 7.09-7.01 (m, 2H), 4.27 (d, J = 13.5 Hz, 2H), 4.19 (d, J = 13.2 Hz, 2H), 3.86-3.80 (m, 2H), 3.39 (d, J = 7.2 Hz, 1H), 2.81 (t, J = 12.0 Hz, 2H), 2.30 (s, 4H), 2.12 (d, J = 7.0 Hz, 2H), 1.95 (dd, J = 13.5, 4.7 Hz, 2H), 1.78 (t, J = 8.1 Hz, 8H), 1.12 (d, J = 7.0 Hz, 4H), 1.06 (t, J = 6.8 Hz, 6H), 0.96-0.88 (m, 3H), 0.72 (d, J = 6.2 Hz, 2H). | 10-1 |
| 45 | 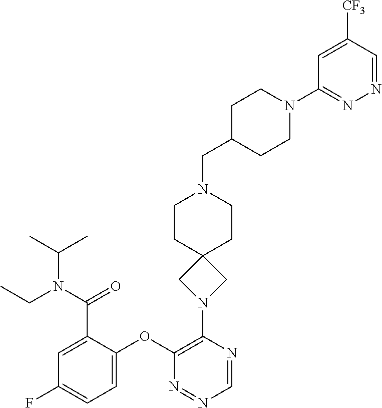 [SMILES: CCN(C(=O)c1cc(F)ccc1Oc1nncnc1N1CC2(CCN(CC3CCN(c4cc(C(F)(F)F)cnn4)CC3)CC2)C1)C(C)C] | 672.4 | ¹H NMR (400 MHz, CD₃OD) δ 8.57 (s, 1H), 8.29 (s, 1H), 7.39 (s, 1H), 7.34-7.30 (m, 1H), 7.21-7.16 (m, 1H), 7.12 (dd, J = 8.0, 2.9 Hz, 1H), 4.41 (d, J = 13.4 Hz, 2H), 4.28 (d, J = 13.6 Hz, 2H), 3.84 (d, J = 8.0 Hz, 2H), 3.76- 3.65 (m, 1H), 3.46-3.33 (m, 1H), 2.95 (t, J = 11.2 Hz, 2H), 2.37 (s, 4H), 2.13 (t, J = 6.6 Hz, 2H), 1.80 (dd, J = 10.6, 7.8 Hz, 8H), 1.06 (t, J = 6.8 Hz, 6H), 1.03-0.79 (m, 3H), 0.72 (d, J = 6.3 Hz, 2H). | 10-1 |
| 46 | 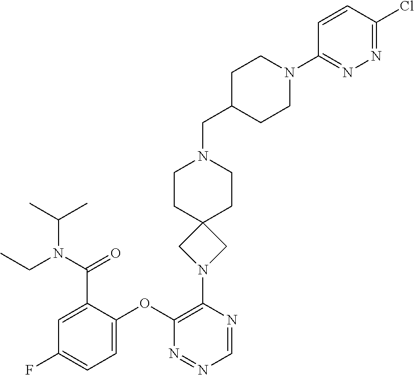 [SMILES: CCN(C(=O)c1cc(F)ccc1Oc1nncnc1N1CC2(CCN(CC3CCN(c4ccc(Cl)nn4)CC3)CC2)C1)C(C)C] | 638.4 | ¹H NMR (400 MHz, DMSO-d₆) δ 8.42 (s, 1H), 7.44 (d, J = 9.6 Hz, 1H), 7.43-7.38 (m, 1H), 7.34-7.31 (m, 3H), 4.25 (s, 2H), 4.18 (s, 2H), 3.77 (s, 2H), 3.06- 3.01 (m, 2H), 2.85 (d, J = 12.0 Hz, 2H), 2.30 (s, 4H), 2.05 (s, 2H), 1.73 (d, J = 13.7 Hz, 8H), 1.09-1.02 (m, 6H), 1.00-0.90 (m, 3H), 0.68 (d, J = 5.9 Hz, 2H). | 10-1 |
| 47 | 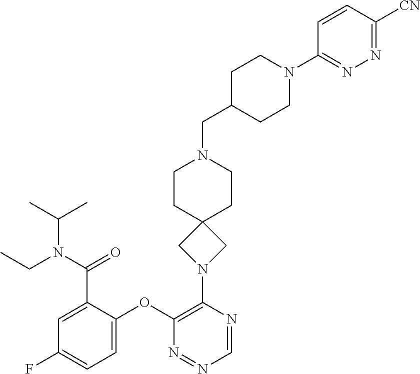 [SMILES: CCN(C(=O)c1cc(F)ccc1Oc1nncnc1N1CC2(CCN(CC3CCN(c4ccc(C#N)nn4)CC3)CC2)C1)C(C)C] | 629.6 | ¹H NMR (400 MHz, a mixture of CD₃OD and CDCl₃) δ 8.34 (s, 1H), 7.55 (d, J = 9.7 Hz, 1H), 7.34-7.29 (m, 1H), 7.22-7.17 (m, 1H), 7.15-7.03 (m, 2H), 4.52 (d, J = 12.8 Hz, 2H), 4.40-4.25 (m, 2H), 3.95-3.87 (m, 2H), 3.84-3.78 (m, 1H), 3.52- 3.42 (m, 1H), 3.25- 3.12 (m, 1H), 3.05 (t, J = 11.9 Hz, 2H), 2.60- 2.25 (br, 4H), 2.20 (d, J = 6.5 Hz, 2H), 1.94- 1.83 (m, 7H), 1.27-1.20 (m, 2H), 1.19-1.16 (m, 2H), 1.15-1.10 (m, 4H), 1.05 (t, J = 7.0 Hz, 1H), 0.79 (d, J = 6.0 Hz, 2H). | 10-1 |
| 48 | 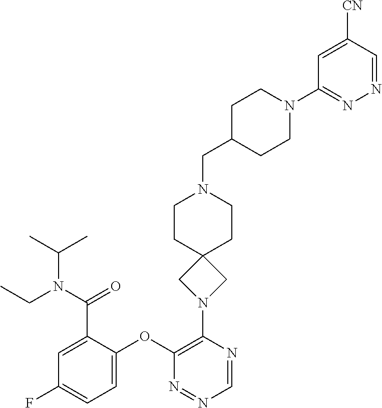 [SMILES: CCN(C(=O)c1cc(F)ccc1Oc1nncnc1N1CC2(CCN(CC3CCN(c4cc(C#N)cnn4)CC3)CC2)C1)C(C)C] | 629.4 | ¹H NMR (400 MHz, CD₃OD) δ 8.50 (d, J = 1.3 Hz, 1H), 8.29 (d, J = 0.9 Hz, 1H), 7.56 (d, J = 0.5 Hz, 1H), 7.34- 7.30 (m, 1H), 7.21-7.16 (m, 1H), 7.11 (dd, J = 7.8, 2.8 Hz, 1H), 4.35 (d, J = 13.3 Hz, 2H), 4.27 (d, J = 13.9 Hz, 2H), 3.83 (d, J = 7.8 Hz, 2H), 3.41- 3.39 (m, 1H), 3.14-3.11 (m, 1H), 2.91 (d, J = 12.5 Hz, 2H), 2.36 (s, 4H), 2.12 (d, J = 6.6 Hz, 2H), 1.83-1.76 (m, 8H), 1.09- 1.05 (m, 6H), 0.99-0.95 (m, 3H), 0.71 (d, J = 6.4 Hz, 2H). | 10-1 |
| 49 | 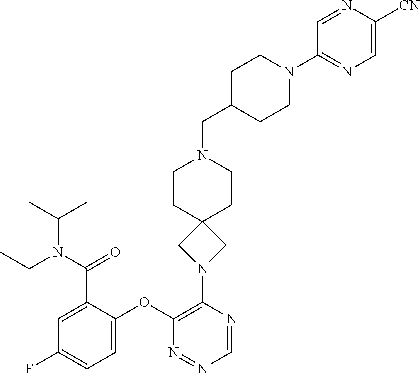 [SMILES: CCN(C(=O)c1cc(F)ccc1Oc1nncnc1N1CC2(CCN(CC3CCN(c4cnc(C#N)cn4)CC3)CC2)C1)C(C)C] | 629.4 | ¹H NMR (400 MHz, DMSO-d₆) δ 8.49 (s, 1H), 8.43 (s, 1H), 8.39 (s, 1H), 7.41 (d, J = 4.7 Hz, 1H), 7.35-7.31 (m, 2H), 4.45 (d, J = 12.7 Hz, 2H), 4.19 (s, 2H), 3.78 (s, 2H), 3.62-3.55 (m, 1H), 2.99- 2.95 (m, 3H), 2.30 (s, 4H), 2.07 (d, J = 6.9 Hz, 2H), 1.78-1.73 (m, 8H), 1.06-0.92 (m, 9H), 0.69 (d, J = 6.1 Hz, 2H). | 10-1 |
| 50 | 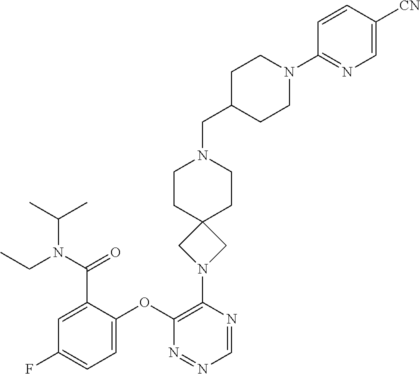 [SMILES: CCN(C(=O)c1cc(F)ccc1Oc1nncnc1N1CC2(CCN(CC3CCN(c4ccc(C#N)cn4)CC3)CC2)C1)C(C)C] | 628.4 | ¹H NMR (400 MHz, CD₃OD) δ 8.38 (d, J = 0.9 Hz, 1H), 8.35 (d, J = 2.4 Hz, 1H), 7.69-7.62 (m, 1H), 7.44-7.37 (m, 1H), 7.31- 7.18 (m, 2H), 6.82 (d, J = 9.2 Hz, 1H), 4.54-4.44 (m, 2H), 4.42-4.31 (m, 2H), 3.96-3.86 (m, 2H), 3.84-3.75 (m, 1H), 3.57- 3.43 (m, 1H), 3.28-3.17 (m, 1H), 3.02-2.90 (m, 2H), 2.55-2.33 (m, 3H), 2.21 (d, J = 6.7 Hz, 2H), 1.89-1.85 (m, 6H), 1.32- 1.27 (m, 2H), 1.21 (d, J = 6.8 Hz, 2H), 1.17-1.12 (m, 6H), 1.06 (t, J = 7.0 Hz, 1H), 0.83-0.77 (m, 2H). | 10-1 |
| 51 | 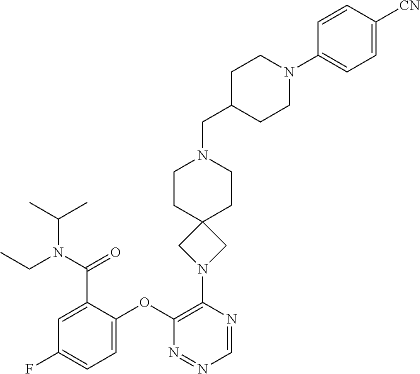 [SMILES: CCN(C(=O)c1cc(F)ccc1Oc1nncnc1N1CC2(CCN(CC3CCN(c4ccc(C#N)cc4)CC3)CC2)C1)C(C)C] | 627.4 | ¹H NMR (400 MHz, CD₃OD) δ 8.38 (s, 1H), 7.47 (d, J = 8.9 Hz, 2H), 7.43-7.38 (m, 1H), 7.32-7.17 (m, 2H), 6.97 (d, J = 9.0 Hz, 2H), 4.45-4.20 (m, 2H), 4.01-3.87 (m, 4H), 3.86-3.75 (m, 1H), 3.58-3.44 (m, 1H), 3.28-3.15 (m, 1H), 2.86 (t, J = 11.8 Hz, 2H), 2.65-2.25 (br, 4H), 2.21 (d, J = 6.5 Hz, 2H), 1.90-1.80 (m, 7H), 1.32- 1.25 (m, 3H), 1.24-1.18 (m, 2H), 1.17-1.12 (m, 4H), 1.06 (t, J = 7.1 Hz, 1H), 0.80 (d, J = 6.3 Hz, 2H). | 10-1 |
| 52 | 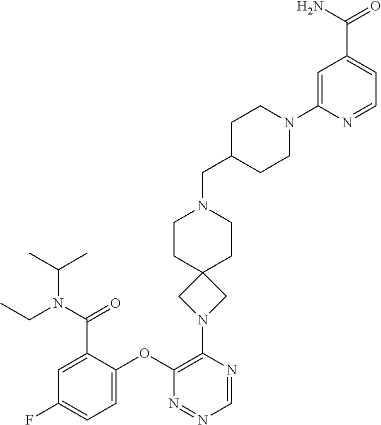 [SMILES: CCN(C(=O)c1cc(F)ccc1Oc1nncnc1N1CC2(CCN(CC3CCN(c4cc(C(N)=O)ccn4)CC3)CC2)C1)C(C)C] | 646.6 | ¹H NMR (400 MHz, CD₃OD) δ 8.37 (d, J = 0.6 Hz, 1H), 8.14 (d, J = 5.3 Hz, 1H), 7.42-7.38 (m, 1H), 7.31-7.16 (m, 3H), 6.94 (d, J = 5.3 Hz, 1H), 4.40-4.30 (m, 4H), 3.95-3.88 (m, 2H), 3.84-3.75 (m, 1H), 3.56-3.43 (m, 1H), 3.26-3.18 (m, 1H), 2.89 (t, J = 11.9 Hz, 2H), 2.60-2.26 (m, 4H), 2.20 (d, J = 6.5 Hz, 2H), 1.90-1.86 (m, 5H), 1.85-1.80 (m, 2H), 1.25-1.17 (m, 3H), 1.17-1.12 (m, 5H), 1.05 (t, J = 7.1 Hz, 1H), 0.80 (d, J = 6.2 Hz, 2H). | 10-1 |
| 53 | 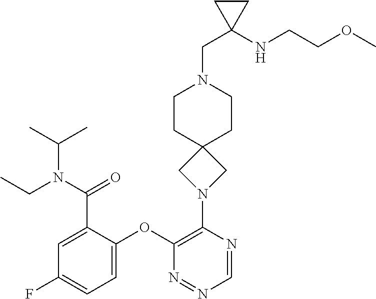 [SMILES: CCN(C(=O)c1cc(F)ccc1Oc1nncnc1N1CC2(CCN(CC3(NCCOC)CC3)CC2)C1)C(C)C] | 556.8 | ¹H NMR (400 MHz, CD₃OD) δ 8.37 (s, 1H), 7.44-7.37 (m, 1H), 7.30-7.17 (m, 2H), 4.41- 4.22 (m, 2H), 3.95-3.84 (m, 2H), 3.84-3.73 (m, 1H), 3.56-3.47 (m, 1H), 3.46 (t, J = 5.2 Hz, 2H), 3.34 (s, 3H), 3.27- 3.13 (m, 1H), 2.78 (t, J = 5.3 Hz, 2H), 2.62- 2.40 (br, 3H), 2.34 (s, 2H), 2.21-1.98 (m, 1H), 1.86-1.81 (br, 4H), 1.23- 1.13 (m, 6H), 1.06 (t, J = 7.1 Hz, 1H), 0.82-0.78 (m, 2H), 0.69-0.60 (m, 2H), 0.39-0.36 (m, 2H). | 10-3 |
| 54 | 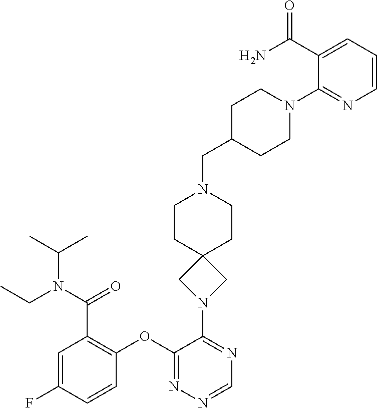 [SMILES: CCN(C(=O)c1cc(F)ccc1Oc1nncnc1N1CC2(CCN(CC3CCN(c4ncccc4C(N)=O)CC3)CC2)C1)C(C)C] | 646.4 | ¹H NMR (400 MHz, CD₃OD) δ 8.42 (s, 1H), 7.93 (s, 1H), 7.68 (s, 1H), 7.43-7.36 (m, 1H), 7.33- 7.19 (m, 2H), 4.68-4.39 (m, 3H), 4.30-4.13 (m, 1H), 4.08-3.96 (m, 2H), 3.91 (s, 3H), 3.87-3.76 (m, 1H), 3.53-3.41 (m, 1H), 3.27-2.97 (m, 6H), 2.92- 2.86 (m, 2H), 2.26-2.05 (m, 5H), 1.92-1.80 (m, 2H), 1.35-1.21 (m, 3H), 1.20-1.04 (m, 7H), 0.85-1.73 (m, 2H). | 10-1 |

### Compound 55: 2-((5-(7-((1-([1,2,4]triazolo[1,5-a]pyridin-7-yl)piperidin-4-yl)methyl)-2,7-diazaspiro[3.5]nonan-2-yl)-1,2,4-triazin-6-yl)oxy)-N-ethyl-5-fluoro-N-isopropylbenizamide

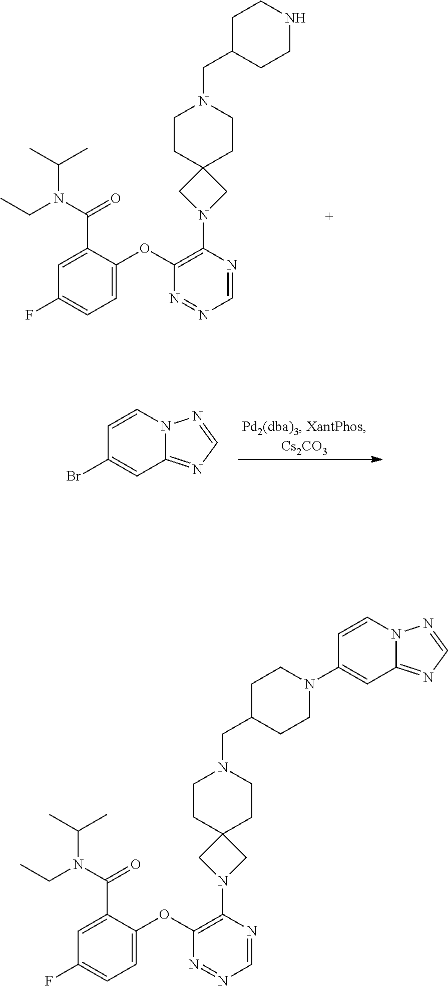 [SMILES: CCN(C(=O)c1cc(F)ccc1Oc1nncnc1N1CC2(CCN(CC3CCNCC3)CC2)C1)C(C)C ; Brc1ccn2ncnc2c1 ; "Pd2(dba)3, XantPhos,Cs2CO3" ; CCN(C(=O)c1cc(F)ccc1Oc1nncnc1N1CC2(CCN(CC3CCN(c4ccn5ncnc5c4)CC3)CC2)C1)C(C)C]

To a reaction flask were added successively intermediate 10-1 (100 mg, 0.19 mmol), 7-bromo-[1,2,4]triazolo[1,5-a]pyridine (56 mg, 0.28 mmol), Pd₂(dba)₃ (17 mg, 0.019 mmol), Xantphos (22 mg, 0.038 mmol), cesium carbonate (155 mg, 0.48 mmol) and dioxane (25 ml), and the mixture was heated to 100° C. and reacted for 15 hours under nitrogen protection. After the reaction was completed, the reaction solution was concentrated, and the residue was purified with flash column chromatography (eluted with a gradient of water/methanol=100:0-0:100) to obtain 25 mg of white solid. MS (m/z): 643.4 [M+H]⁺.

¹H NMR (400 MHz, CD₃OD) δ 8.45-8.42 (m, 2H), 8.32 (s, 1H), 8.15 (s, 1H), 7.43-7.38 (m, 1H), 7.33-7.20 (m, 2H), 7.03-7.01 (m, 1H), 6.83 (d, J=2.3 Hz, 1H), 4.47 (s, 2H), 4.08-3.96 (m, 4H), 3.86-3.79 (m, 1H), 3.56-3.42 (m, 1H), 3.28-3.08 (br, 4H), 3.02-2.95 (m, 4H), 2.20-2.11 (m, 5H), 1.94 (d, J=12.5 Hz, 2H), 1.48-1.36 (m, 2H), 1.20-1.12 (m, 6H), 1.07 (t, J=7.1 Hz, 1H), 0.80 (d, J=6.0 Hz, 2H).

The following compounds were prepared according to the preparation procedure of compound 55 using corresponding intermediates and reagents under appropriate conditions that will be recognized by those skilled in the art.

| | | MS | | Intermediate |
|---|---|---|---|---|
| Compound | Structural formula | (M+H)+ | 1HNMR | used |
| 56 | 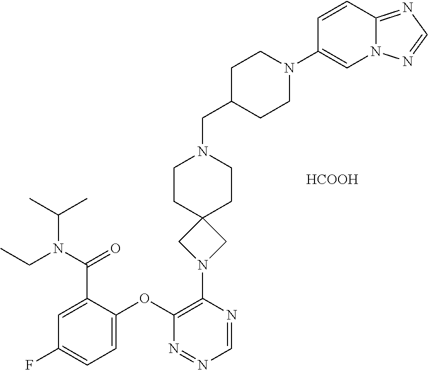 [SMILES: CCN(C(=O)c1cc(F)ccc1Oc1nncnc1N1CC2(CCN(CC3CCN(c4ccc5ncnn5c4)CC3)CC2)C1)C(C)C ; "HCOOH"] | 643.4 | ¹H NMR (400 MHz, CD₃OD) δ 8.42 (s, 2H), 8.27 (s, 1H), 8.22 (s, 1H), 7.68-7.61 (m, 2H), 7.40 (d, J = 4.1 Hz, 1H), 7.35-7.17 (m, 2H), 4.46 (s, 2H), 4.02 (s, 2H), 3.88-3.77 (m, 1H), 3.68 (d, J = 11.9 Hz, 2H), 3.55-3.42 (m, 1H), 3.23-3.00 (m, 5H), 2.89 (d, J = 5.7 Hz, 2H), 2.77 (t, J = 11.6 Hz, 2H), 2.20-2.06 (m, 4H), 2.03-1.91 (m, 3H), 1.58-1.42 (m, 2H), 1.23- 1.05 (m, 7H), 0.85-0.76 (m, 2H). | 10-1 |

### Compound 57: N-ethyl-5-fluoro-N-isopropyl-2-((5-(7-((1-(pyridin-2-yl)piperidin-4-yl)methyl)-2,7-diazaspiro[3.5]nonan-2-yl)-1,2,4-triazin-6-yl)oxy)benzamide

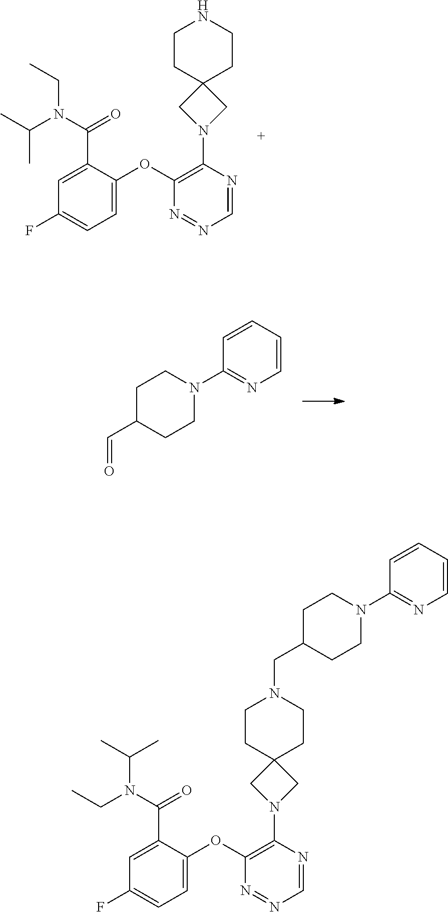 [SMILES: CCN(C(=O)c1cc(F)ccc1Oc1nncnc1N1CC2(CCNCC2)C1)C(C)C ; O=CC1CCN(c2ccccn2)CC1 ; CCN(C(=O)c1cc(F)ccc1Oc1nncnc1N1CC2(CCN(CC3CCN(c4ccccn4)CC3)CC2)C1)C(C)C]

In a reaction flask, intermediate 7-1 (43 mg, 0.1 mmol) and intermediate 12-1 (38 mg, 0.2 mmol) were dissolved in 20 ml of methanol, and then sodium cyanoborohydride (63 mg, 1.0 mmol) was added to the solution. The reaction solution was stirred for 15 hours at room temperature. After the reaction was completed, the reaction solution was concentrated, and the residue was purified with flash column chromatography (eluted with a gradient of water/methanol=100:0-0:100) to obtain 19 mg of product. MS (m/z): 603.4 [M+H]⁺.

¹H NMR (400 MHz, CD₃OD) δ 8.38 (s, 1H), 8.34 (s, 1H), 7.38-7.35 (m, 1H), 7.28-7.20 (m, 1H), 7.18-7.12 (m, 1H), 4.45-4.16 (m, 2H), 3.95 (d, J=13.2 Hz, 4H), 3.87-3.75 (m, 1H), 3.52-3.33 (m, 1H), 3.18-3.08 (m, 1H), 2.90-2.69 (m, 6H), 2.60-2.47 (i, 3H), 1.98 (t, J=5.5 Hz, 4H), 1.92-1.82 (m, 1H), 1.75 (d, J=13.1 Hz, 2H), 1.25-1.01 (m, 9H), 0.95-0.75 (i, 2H), 0.67-0.59 (i, 2H), 0.46-0.39 (m, 2H).

The following compounds were prepared according to the preparation procedure of compound 57 using corresponding intermediates and reagents under appropriate conditions that will be recognized by those skilled in the art.

| | | MS | | Inter- |
|---|---|---|---|---|
| Com- | | (M + | | mediate |
| pound | Structural formula | H)⁺ | ¹HNMR | used |
| 58 | 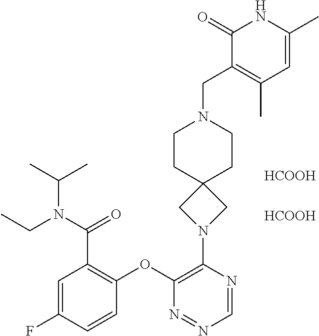 [SMILES: CCN(C(=O)c1cc(F)ccc1Oc1nncnc1N1CC2(CCN(Cc3c(C)cc(C)[nH]c3=O)CC2)C1)C(C)C ; "HCOOH" ; "HCOOH"] | 564.6 | ¹H NMR (400 MHz, CD₃OD) δ 8.45 (s, 2H), 8.42 (s, 1H), 7.43-7.40 (m, 1H), 7.33-7.17 (m, 2H), 6.18 (s, 1H), 4.47 (s, 2H), 4.24-4.12 (m, 2H), 4.04 (s, 2H), 3.84-3.81 (m, 1H), 3.49-3.45 (m, 1H), 3.31-3.25 (m, 5H), 2.32 (s, 3H), 2.27 (s,3H), 2.18-2.12 (m, 4H), 1.16- 1.10 (m, 7H), 0.84-0.78 (m, 2H). | 7-1 |
| 59 | 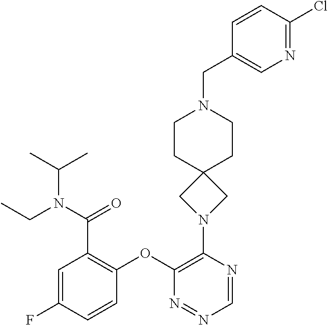 [SMILES: CCN(C(=O)c1cc(F)ccc1Oc1nncnc1N1CC2(CCN(Cc3ccc(Cl)nc3)CC2)C1)C(C)C] | 554.2 | ¹H NMR (400 MHz, DMSO-d6) δ 8.42 (d, J = 0.6 Hz, 1H), 8.28 (d, J = 2.3 Hz, 1H), 7.74 (dd, J = 8.2, 2.3 Hz, 1H), 7.45 (d, J = 8.2 Hz, 1H), 7.41 (dd, J = 6.3, 3.6 Hz, 1H), 7.33 (td, J = 8.0, 3.2 Hz, 2H), 4.22-4.18 (m, 2H), 3.79 (d, J = 7.1 Hz, 2H), 3.62-3.54 (m, 1H), 3.45 (s, 2H), 3.12-2.99 (m, 1H), 2.96 (s, 1H), 2.30- 2.26 (m, 4H), 1.72 (s, 4H), 1.05-0.88 (m, 6H), 0.87-0.65 (m, 3H). | 7-1 |
| 60 | 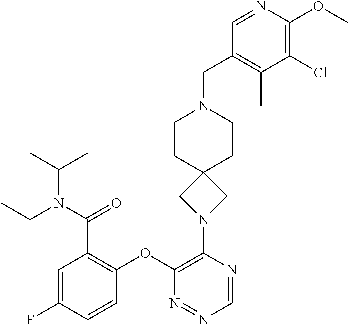 [SMILES: CCN(C(=O)c1cc(F)ccc1Oc1nncnc1N1CC2(CCN(Cc3cnc(OC)c(Cl)c3C)CC2)C1)C(C)C] | 598.2 | ¹H NMR (400 MHz, DMSO-d6) δ 8.42 (s, 1H), 7.85 (s, 1H), 7.44-7.39 (m, 1H), 7.35-7.30 (m, 2H), 4.19 (s, 2H), 3.87 (s, 3H), 3.78 (s, 2H), 3.64-3.55 (m, 1H), 3.36 (s, 2H), 3.07 (s, 1H), 2.98 (s, 1H), 2.36 (s, 3H), 2.31 (s, 4H), 1.68 (s, 4H), 1.06-0.95 (m, 6H), 0.81-0.75 (m, 3H). | 7-1 and 34 |
| 61 | 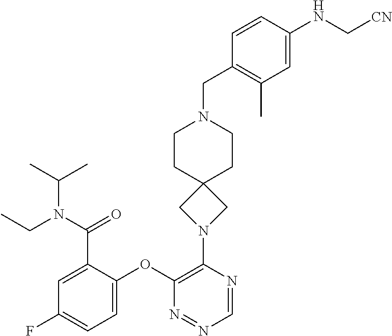 [SMILES: CCN(C(=O)c1cc(F)ccc1Oc1nncnc1N1CC2(CCN(Cc3ccc(NCC#N)cc3C)CC2)C1)C(C)C] | 587.6 | ¹H NMR (400 MHz, CD₃OD) δ 8.37 (s, 1H), 7.44-7.38 (m, 1H), 7.31-7.16 (m, 2H), 7.07 (d, J = 8.2 Hz, 1H), 6.60-6.48 (m, 2H), 4.42-4.30(m, 2H), 4.15 (s, 2H), 3.96-3.86 (m, 2H), 3.85-3.75 (m, 1H), 3.57-3.42 (m, 1H), 3.38 (m, 2H), 3.27- 3.15 (m, 1H), 2.65-2.33 (br, 4H), 2.32 (s, 3H), 1.87-1.80 (m, 4H), 1.22-1.11 (m, 6H), 1.05 (t, J = 7.1 Hz, 1H), 0.80 (d, J = 6.1 Hz, 2H). | 7-1 and 20 |
| 62 | 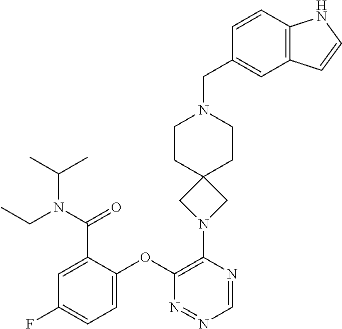 [SMILES: CCN(C(=O)c1cc(F)ccc1Oc1nncnc1N1CC2(CCN(Cc3ccc4[nH]ccc4c3)CC2)C1)C(C)C] | 558.3 | ¹H NMR (400 MHz, DMSO-d6) δ 11.01 (s, 1H), 8.42 (s, 1H), 8.15 (s, 1H), 7.39 (s, 1H), 7.34-7.28 (m, 4H), 7.00 (dd, J = 8.3, 1.4 Hz, 1H), 6.34 (t, J = 2.1 Hz, 1H), 4.17 (s, 2H), 3.79 (d, J = 7.8 Hz, 2H), 3.58-3.54 (m, 1H), 3.52 (s, 2H), 3.36-3.27 (m, 1H), 3.09-2.98 (m, 1H), 2.40-2.30 (m, 4H), 1.72 (s, 4H), 1.05- 0.98 (m, 6H), 0.84-0.64 (m, 3H). | 7-1 |
| 63 | 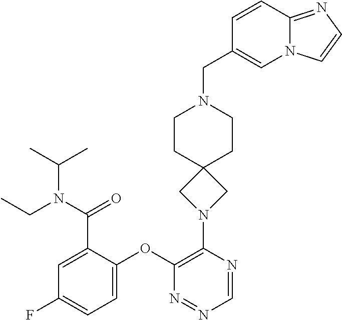 [SMILES: CCN(C(=O)c1cc(F)ccc1Oc1nncnc1N1CC2(CCN(Cc3ccc4nccn4c3)CC2)C1)C(C)C] | 559.3 | ¹H NMR (400 MHz, DMSO-d6) δ 8.44- 8.40 (m, 2H), 7.52 (d, J = 9.1 Hz, 1H), 7.44 (s, 1H), 7.40 (dd, J = 10.8, 6.0 Hz, 1H), 7.36-7.30 (m, 2H), 7.24-7.19 (m, 1H), 6.90 (t, J = 6.8 Hz, 1H), 4.20 (d, J = 15.6 Hz, 2H), 3.76 (s, 4H), 3.63-3.51 (m, 1H), 3.16-2.98 (m, 1H), 2.95 (s, 1H), 2.35 (s, 4H), 1.69 (s, 4H), 1.05- 0.95 (m, 6H), 0.86-0.63 (m, 3H). | 7-1 |
| 64 | 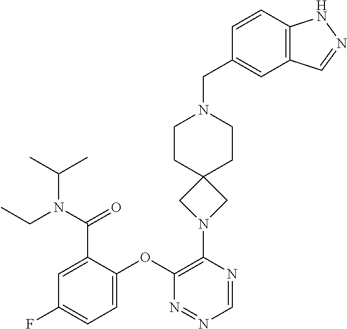 [SMILES: CCN(C(=O)c1cc(F)ccc1Oc1nncnc1N1CC2(CCN(Cc3ccc4[nH]ncc4c3)CC2)C1)C(C)C] | 559.3 | ¹H NMR (400 MHz, CD₃OD) δ 8.38- 8.33 (m, 1H), 8.00 (s, 1H), 7.68 (s, 1H), 7.52-7.48 (m, 1H), 7.43-7.34 (m, 2H), 7.30-7.22 (m, 1H), 7.22-7.14 (m, 1H), 4.43-4.20 (m, 2H), 3.93-3.88 (m, 2H), 3.83-3.71 (m, 1H), 3.62 (s, 2H), 3.46- 3.42 (m, 1H), 3.26-3.13 (m, 1H), 2.57- 2.45 (m, 4H), 1.88-1.83 (m, 4H), 1.20- 0.97 (m, 7H), 0.77-0.72 (m, 2H). | 7-1 |
| 65 | 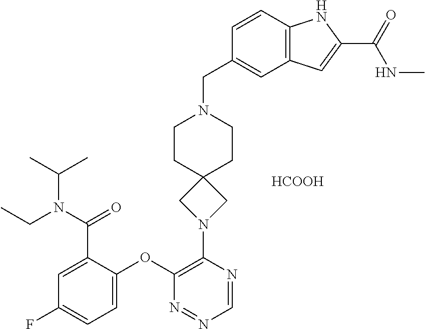 [SMILES: CCN(C(=O)c1cc(F)ccc1Oc1nncnc1N1CC2(CCN(Cc3ccc4[nH]c(C(=O)NC)cc4c3)CC2)C1)C(C)C ; "HCOOH"] | 615.3 | ¹H NMR (400 MHz, CD₃OD) δ 8.42- 8.39 (m, 2H), 7.76 (s, 1H), 7.55-7.52 (m, 1H), 7.41-7.38 (m, 1H), 7.35-7.24 (m, 2H), 7.22-7.18 (m, 1H), 7.07 (s, 1H), 4.46-4.41 (m, 2H), 4.31 (s, 2H), 4.03-3.98 (m, 2H), 3.86-3.74 (m, 1H), 3.45-3.42 (m, 1H), 3.28-3.01 (m, 5H), 2.92 (s, 3H), 2.12-2.07 (m, 4H), 1.18- 1.00 (m, 7H), 0.78-0.73 (m, 2H). | 7-1 |
| 66 | 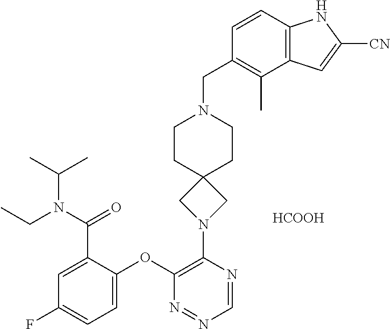 [SMILES: CCN(C(=O)c1cc(F)ccc1Oc1nncnc1N1CC2(CCN(Cc3ccc4[nH]c(C#N)cc4c3C)CC2)C1)C(C)C ; "HCOOH"] | 597.3 | ¹H NMR (400 MHz, DMSO-d6) δ 12.24 (s, 1H), 8.42 (d, J = 0.8 Hz, 1H), 7.41 (s, 2H), 7.33 (dd, J = 13.1, 5.2 Hz, 2H), 7.23-7.17 (m, 2H), 4.19 (s, 2H), 3.78 (s, 2H), 3.60-3.55 (m, 1H), 3.34 (s, 2H), 3.03-2.98 (m, 2H), 2.45 (s, 3H), 2.39-2.36 (m, 4H), 1.68 (s, 4H), 0.99- 0.90 (m, 6H), 0.84-0.66 (m, 3H). | 7-1 |
| 67 | 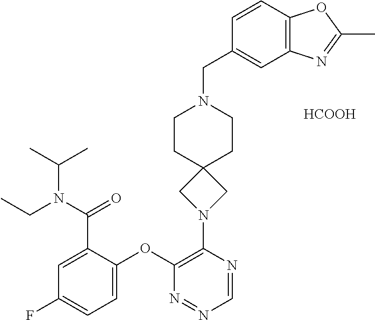 [SMILES: CCN(C(=O)c1cc(F)ccc1Oc1nncnc1N1CC2(CCN(Cc3ccc4oc(C)nc4c3)CC2)C1)C(C)C ; "HCOOH"] | 574.6 | ¹H NMR (400 MHz, CD₃OD) δ 8.46 (s, 1H), 8.39 (s, 1H), 7.68 (s, 1H), 7.59 (d, J = 8.4 Hz, 1H), 7.46-7.35 (m, 2H), 7.34- 7.18 (m, 2H), 4.45-4.32 (m, 2H), 4.03- 3.92 (m, 4H), 3.86-3.75 (m, 1H), 3.52- 3.38 (m, 1H), 3.28-3.09 (m, 1H), 3.01- 2.70 (m, 4H), 2.64 (s, 3H), 2.03-1.92 (br, 4H), 1.22-1.02 (m, 7H), 0.81-0.75 (m, 2H). | 7-1 |
| 68 | 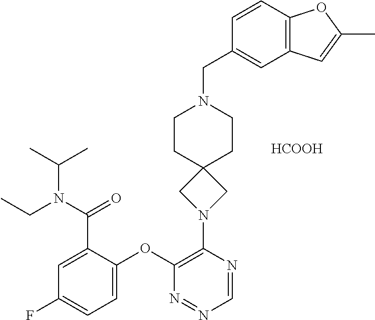 [SMILES: CCN(C(=O)c1cc(F)ccc1Oc1nncnc1N1CC2(CCN(Cc3ccc4oc(C)cc4c3)CC2)C1)C(C)C ; "HCOOH"] | 573.4 | ¹H NMR (400 MHz, CD₃OD) δ 8.40 (s, 2H), 7.60 (s, 1H), 7.48-7.45 (m, 1H), 7.40-7.37 (m, 1H), 7.34-7.24 (m, 2H), 7.23-7.20 (m, 1H), 6.50 (s, 1H), 4.46- 4.43 (m, 2H), 4.26 (s, 2H), 4.03-3.98 (m, 2H), 3.84-3.75 (m, 1H), 3.47-3.43 (m, 1H), 3.22-3.17 (m, 5H), 2.45 (s, 3H), 2.12-2.07 (m, 4H), 1.20-1.03 (m, 7H), 0.79-0.74 (m, 2H). | 7-1 |
| 69 | 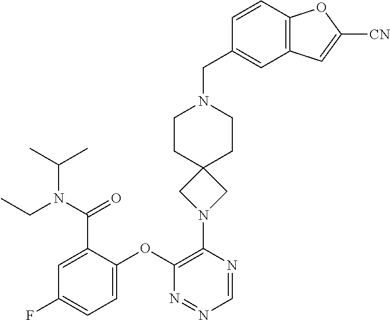 [SMILES: CCN(C(=O)c1cc(F)ccc1Oc1nncnc1N1CC2(CCN(Cc3ccc4oc(C#N)cc4c3)CC2)C1)C(C)C] | 584.4 | ¹H NMR (400 MHz, CD₃OD) δ 8.37 (s, 1H), 7.73 (d, J = 3.4 Hz, 2H), 7.62-7.48 (m, 2H), 7.42-7.35 (m, 1H), 7.31-7.16 (m, 2H), 4.42-4.30 (m, 2H), 3.99-3.86 (m, 3H), 3.79-3.75 (m, 1H), 3.74-3.65 (m, 2H), 3.46 (s, 1H), 3.25-3.14 (m, 1H), 2.55 (s, 2H), 1.95-1.84 (m, 4H), 1.28 (s, 2H), 1.18 (d, J = 6.8 Hz, 1H), 1.4-1.102 (m, 4H), 1.04 (t, J = 7.1 Hz, 1H), 0.77 (d, J = 6.2 Hz, 2H). | 7-1 |
| 70 | 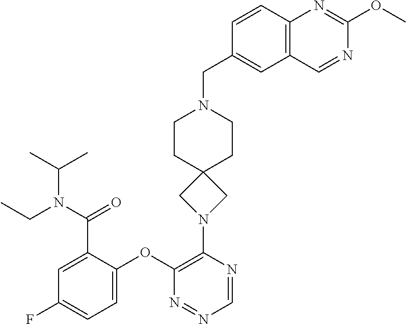 [SMILES: CCN(C(=O)c1cc(F)ccc1Oc1nncnc1N1CC2(CCN(Cc3ccc4nc(OC)ncc4c3)CC2)C1)C(C)C] | 601.4 | ¹H NMR (400 MHz, CD₃OD) δ 9.29 (s, 1H), 8.37 (d, J = 1.0 Hz, 1H), 8.00-7.89 (m, 2H), 7.83-7.73 (m, 1H), 7.44-7.37 (m, 1H), 7.30-7.17 (m, 2H), 4.40-4.34 (m, 2H), 4.10 (s, 3H), 3.95-3.88 (m, 2H), 3.84-3.75 (m, 1H), 3.68 (s, 2H), 3.50- 3.42 (m, 1H), 3.25-3.16 (m, 1H), 2.60- 2.36 (m, 3H), 1.91-1.85 (m, 4H), 1.35- 1.27 (m, 2H), 1.18 (d, J = 6.8 Hz, 1H), 1.15-1.11 (m, 4H), 1.03 (t, J = 7.1 Hz, 1H), 0.77 (d, J = 6.4 Hz, 2H). | 7-1 and 30 |
| 71 | 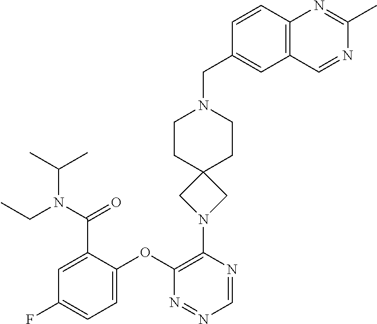 [SMILES: CCN(C(=O)c1cc(F)ccc1Oc1nncnc1N1CC2(CCN(Cc3ccc4nc(C)ncc4c3)CC2)C1)C(C)C] | 585.4 | ¹H NMR (400 MHz, CD₃OD) δ 9.41 (s, 1H), 8.53 (s, 1H), 8.37 (s, 1H), 8.04- 7.97 (m, 2H), 7.89 (d, J = 8.6 Hz, 1H), 7.40 (dd, J = 9.1, 4.6 Hz, 1H), 7.34- 7.13 (m, 2H), 4.45-4.21 (m, 2H), 3.97- 3.88 (m, 2H), 3.83-3.76 (m, 1H), 3.72 (s, 2H), 3.56-3.40 (m, 1H), 3.27-3.15 (m, 1H), 2.82 (s, 3H), 2.63-2.35 (m, 4H), 1.92-1.84 (m, 4H), 1.18 (d, J = 6.8 Hz, 2H), 1.15-1.09 (m, 4H), 1.04 (t, J = 7.1 Hz, 1H), 0.78 (d, J = 6.3 Hz, 2H). | 7-1 and 31 |
| 72 | 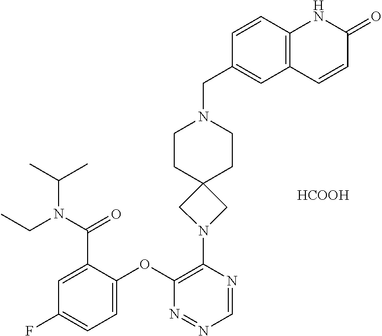 [SMILES: CCN(C(=O)c1cc(F)ccc1Oc1nncnc1N1CC2(CCN(Cc3ccc4[nH]c(=O)ccc4c3)CC2)C1)C(C)C ; "HCOOH"] | 586.8 | ¹H NMR (400 MHz, CD₃OD) δ 8.40 (s, 2H), 7.97 (d, J = 9.5 Hz, 1H), 7.74 (s, 1H), 7.62 (d, J = 8.3 Hz, 1H), 7.42-7.38 (m, 2H), 7.32-7.18 (m, 2H), 6.64 (d, J = 9.5 Hz, 1H), 4.42 (s, 2H), 4.05 (s, 2H), 3.99-3.96 (m, 2H), 3.86-3.77 (m, 1H), 3.48-3.45 (m, 1H), 3.02-2.95 (m, 5H), 2.05-2.00 (m, 4H), 1.16-1.05 (m, 7H), 0.80-0.75 (m, 2H). | 7-1 |
| 73 | 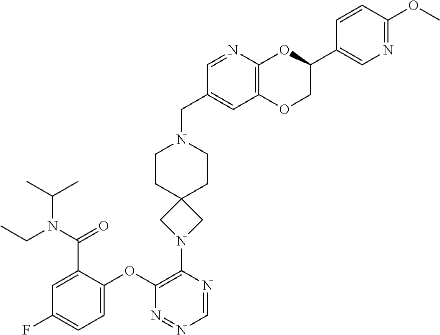 [SMILES: CCN(C(=O)c1cc(F)ccc1Oc1nncnc1N1CC2(CCN(Cc3cnc4c(c3)OC[C@H](c3ccc(OC)nc3)O4)CC2)C1)C(C)C] | 685.3 | ¹H NMR (400 MHz, DMSO-d6) δ 8.43 (d, J = 0.7 Hz, 1H), 8.27 (d, J = 2.4 Hz, 1H), 7.80 (dd, J = 8.2, 2.0 Hz, 1H), 7.67 (d, J = 1.4 Hz, 1H), 7.44-7.39 (m, 1H), 7.35-7.31 (m, 2H), 7.25 (d, J = 1.4 Hz, 1H), 6.88 (d, J = 8.6 Hz, 1H), 5.41 (dd, J = 8.4, 2.1 Hz, 1H), 4.40 (dd, J = 11.2, 2.1 Hz, 1H), 4.21-4.16 (m, 3H), 3.85 (d, J = 0.6 Hz, 3H), 3.78 (s, 2H), 3.64- 3.53 (m, 1H), 3.38 (s, 2H), 3.08-3.02 (m, 2H), 2.33 (s, 4H), 1.73 (s, 4H), 1.05- 0.90 (m, 6H), 0.68 (d, J = 5.8 Hz, 3H). | 7-1 and 33 |
| 74 | 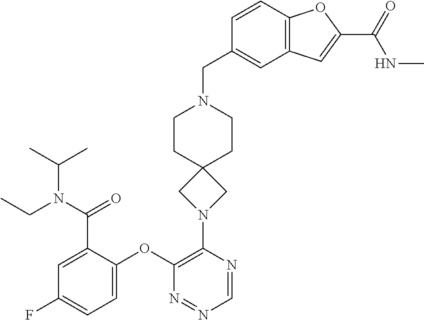 [SMILES: CCN(C(=O)c1cc(F)ccc1Oc1nncnc1N1CC2(CCN(Cc3ccc4oc(C(=O)NC)cc4c3)CC2)C1)C(C)C] | 616.6 | ¹H NMR (400 MHz, CD₃OD) δ 8.37 (s, 1H), 7.68 (s, 1H), 7.55 (d, J = 8.6 Hz, 1H), 7.48 (d, J = 10.5 Hz, 1H), 7.45-7.43 (m, 1H), 7.42-7.37 (m, 1H), 7.31-7.27 (m 1H), 7.27-7.22 (m, 1H), 7.22-7.17 (m, 1H), 454-4.39 (m, 1H), 4.37-4.30 (m, 2H), 3.97-3.88 (m, 2H), 3.84-3.75 (m, 1H), 3.66 (s, 2H), 3.45 (s, 1H), 2.94 (s, 3H), 2.55-2.42 (m, 2H), 1.92-1.85 (m, 4H), 1.30-1.26 (m, 2H), 1.21-1.17 (m, 2H), 1.14-1.11 (m, 4H), 1.04 (t, J = 7.1 Hz, 1H), 0.77 (d, J = 6.1 Hz, 2H). | 7-1 and 39 |
| 75 | 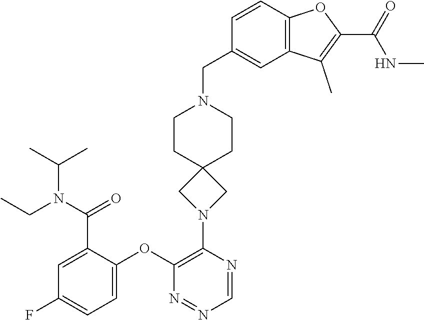 [SMILES: CCN(C(=O)c1cc(F)ccc1Oc1nncnc1N1CC2(CCN(Cc3ccc4oc(C(=O)NC)c(C)c4c3)CC2)C1)C(C)C] | 630.3 | ¹H NMR (400 MHz, CD₃OD) δ 8.37 (s, 1H), 7.63 (s, 1H), 7.49-7.44 (m, 2H), 7.43-7.38 (m, 1H), 7.31-7.24 (m, 1H), 7.23-7.17 (m, 1H), 4.43-4.28 (m, 2H), 3.98-3.87 (m, 2H), 3.84-3.74 (m, 1H), 3.64 (s, 2H), 3.53-3.40 (m, 1H), 3.27- 3.15 (m, 1H), 2.91 (s, 3H), 2.57 (s, 3H), 2.54-2.29 (m, 3H), 1.91-1.85 (m, 4H), 1.34-1.26 (m, 1H), 1.19 (d, J = 6.7 Hz, 2H), 1.15-1.09 (m, 5H), 1.03 (t, J = 7.0 Hz, 1H), 0.77 (d, J = 5.9 Hz, 2H). | 7-1 and 40 |
| 76 | 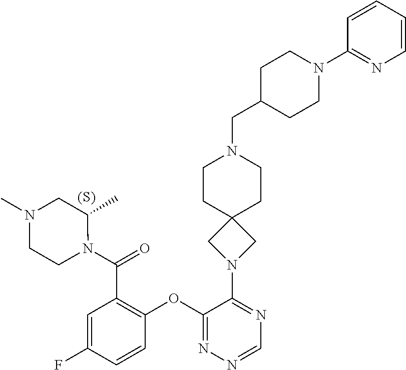 | 630.4 | ¹H NMR (400 MHz, CD₃OD) δ 8.40 (s, 1H), 8.05-8.01 (m, 1H), 7.55-7.48 (m, 1H), 7.46-7.35 (m, 1H), 7.33-7.26 (m, 1H), 7.24-7.12 (m, 1H), 6.80 (d, J = 8.7 Hz, 1H), 6.61 (dd, J = 7.1, 5.1 Hz, 1H), 4.70-4.42 (m, 1H), 4.41-4.29 (m, 2H), 4.25-4.18 (m, 2H), 3.94 (s, 2H), 3.42- 3.34 (m, 1H), 3.19-3.11 (m, 1H), 2.83 (t, J = 11.7 Hz, 3H), 2.75-2.67 (m, 1H), 2.56-2.32 (m, 4H), 2.23 (d, J = 6.2 Hz, 3H), 2.15-2.05 (m, 2H), 1.92-1.82 (m, 7H), 1.36-1.14 (m, 6H), 1.03-0.96 (m, 1H). | 7-5 and 12-1 |
| 77 | 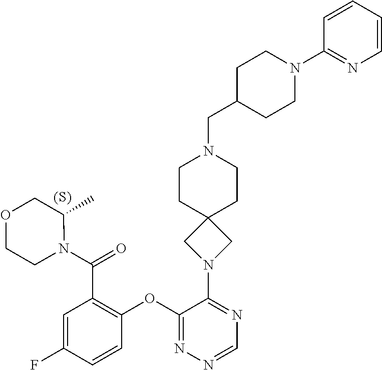 | 617.4 | ¹H NMR (400 MHz, CD₃OD) δ 8.40 (s, 1H), 8.03 (dd, J = 5.0, 2.0 Hz, 1H), 7.55- 7.49 (m, 1H), 7.47-7.35 (m, 1H), 7.33- 7.15 (m, 2H), 6.81 (d, J = 8.4 Hz, 1H), 6.63-6.57 (m, 1H), 4.55-4.29 (m, 3H), 4.25-4.18 (m, 2H), 3.95 (s, 2H), 3.75- 3.61 (m, 2H), 3.59-3.43 (m, 1H), 3.25- 3.13 (m, 1H), 2.94-2.76 (m, 3H), 2.55- 2.36 (m, 3H), 2.24 (d, J = 6.4 Hz, 2H), 1.93-1.78 (m, 8H), 1.39-1.13 (m, 5H), 1.02-0.90 (m, 1H). | 7-6 and 12-1 |
| 78 | 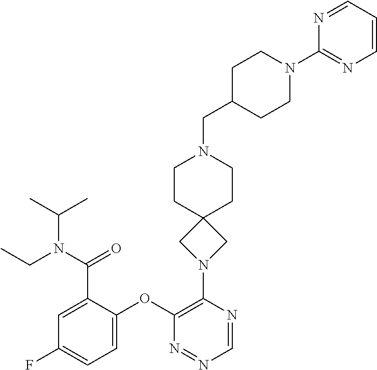 [SMILES: CCN(C(=O)c1cc(F)ccc1Oc1nncnc1N1CC2(CCN(CC3CCN(c4ncccn4)CC3)CC2)C1)C(C)C] | 604.4 | ¹H NMR (400 MHz, DMSO-d6) δ 8.43 (d, J = 0.5 Hz, 1H), 8.30-8.28 (m, 2H), 7.42 (dd, J = 6.2, 3.5 Hz, 1H), 7.34 (td, J = 8.0, 3.3 Hz, 2H), 6.52 (d, J = 4.7 Hz, 1H), 4.61 (d, J = 13.2 Hz, 2H), 4.19 (s, 2H), 3.78 (s, 2H), 3.59 (dd, J = 13.1, 6.6 Hz, 1H), 3.06-3.01 (m, 1H), 2.79 (d, J = 11.1 Hz, 2H), 2.31 (s, 4H), 2.07 (d, J = 6.9 Hz, 2H), 1.72 (s, 8H), 1.06-1.01 (m, 6H), 0.95-0.91 (m, 3H), 0.70 (d, J = 6.0 Hz, 2H). | 7-1 and 12-5 |
| 79 | 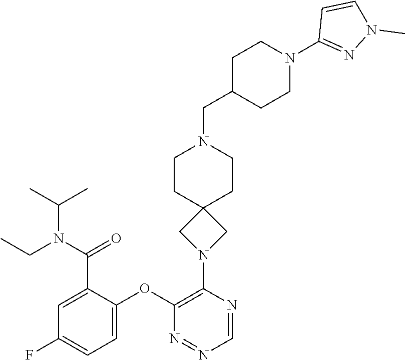 [SMILES: CCN(C(=O)c1cc(F)ccc1Oc1nncnc1N1CC2(CCN(CC3CCN(c4ccn(C)n4)CC3)CC2)C1)C(C)C] | 606.4 | ¹H NMR (400 MHz, CD₃OD) δ 8.37 (s, 1H), 7.42-7.38 (m, 1H), 7.35-7.15 (m, 3H), 5.69 (s, 1H), 4.41-4.25 (m, 2H), 3.96-3.86 (m, 2H), 3.84-3.75 (m, 1H), 3.69 (s, 3H), 3.64 (d, J = 12.3 Hz, 2H), 3.55-3.41 (m, 1H), 3.26-3.18 (m, 1H),2.63 (t, J = 11.6 Hz, 2H), 2.55-2.25 (br, 4H), 2.20 (d, J = 6.9 Hz, 2H), 1.93- 1.76 (m, 6H), 1.74-1.61 (m, 1H), 1.33- 1.24 (m, 2H), 1.20 (d, J = 6.8 Hz, 2H), 1.16-1.10 (m, 4H), 1.05 (t, J = 7.1 Hz, 1H), 0.80 (d, J = 5.8 Hz, 2H). | 7-1 and 32 |
| 80 | 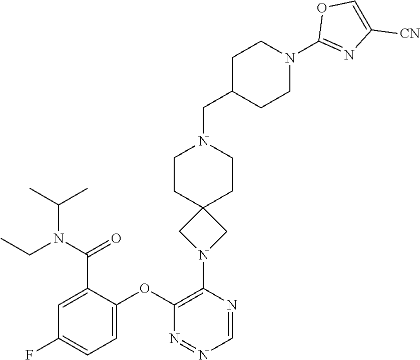 [SMILES: CCN(C(=O)c1cc(F)ccc1Oc1nncnc1N1CC2(CCN(CC3CCN(c4nc(C#N)co4)CC3)CC2)C1)C(C)C] | 618.3 | ¹H NMR (400 MHz, CD₃OD) δ 8.38 (s, 1H), 8.10 (s, 1H), 7.40 (dd, J = 9.1, 4.6 Hz, 1H), 7.30-7.25 (m, 1H), 7.24-7.18 (m, 1H), 4.36 (d, J = 13.4 Hz, 2H), 4.02 (d, J = 13.2 Hz, 2H), 3.92 (d, J = 7.9 Hz, 2H), 3.80 (dd, J = 13.3, 6.7 Hz, 1H), 3.24 (d, J = 6.7 Hz, 1H), 3.06-2.99 (m, 2H), 2.44 (s, 4H), 2.19 (d, J = 7.1 Hz, 2H), 1.85 (d, J = 12.8 Hz, 8H), 1.15 (t, J = 6.9 Hz, 6H), 0.92-0.88 (m, 3H), 0.80 (d, J = 6.3 Hz, 2H). | 7-1 and 18-2 |
| 81 | 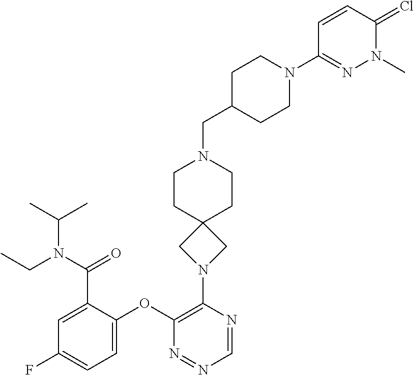 | 634.4 | ¹H NMR (400 MHz, CD₃OD) δ 8.28 (s, 1H), 7.33 (d, J = 10.0 Hz, 1H), 7.31- 7.27 (m, 1H), 7.16 (td, J = 8.1, 2.5 Hz, 1H), 7.09-7.04 (m, 1H), 6.74 (d, J = 9.9 Hz, 1H), 4.26 (s, 2H), 3.82 (d, J = 13.2 Hz, 4H), 3.74 (dd, J = 13.2, 6.7 Hz, 1H), 3.53 (s, 3H), 3.45-3.23 (m, 2H), 3.05 (s, 1H), 2.69 (t, J = 11.6 Hz, 2H), 2.33 (s, 4H), 2.13 (d, J = 6.8 Hz, 2H), 1.76- 1.72 (m, 8H), 1.20-1.11 (m, 6H), 1.07 (d, J = 6.9 Hz, 3H). | 7-1 and 24-4 |
| 82 | 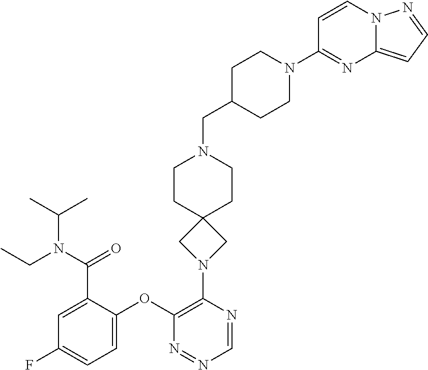 [SMILES: CCN(C(=O)c1cc(F)ccc1Oc1nncnc1N1CC2(CCN(CC3CCN(c4ccn5nccc5n4)CC3)CC2)C1)C(C)C] | 643.4 | ¹H NMR (400 MHz, CD₃OD) δ 8.38 (s, 1H), 8.35 (d, J = 7.9 Hz, 1H), 7.80 (d, J = 2.3 Hz, 1H), 7.43-7.37 (m, 1H), 7.31- 7.17 (m, 2H), 6.65 (d, J = 8.0 Hz, 1H), 6.02 (d, J = 2.2 Hz, 1H), 4.51-4.16 (m, 5H), 3.96-3.88 (m, 2H), 3.86-3.77 (m, 1H), 3.59-3.44 (m, 1H), 3.27-3.14 (m, 1H), 2.99 (t, J = 12.0 Hz, 2H), 2.52-2.39 (m, 2H), 2.26-2.18 (m, 2H), 1.99-1.83 (m, 8H), 1.32-1.26 (m, 1H), 1.21 (d, J = 6.6 Hz, 2H), 1.17-1.12 (m, 5H), 1.06 (t, J = 7.1 Hz, 1H), 0.80 (d, J = 6.1 Hz, 2H). | 7-1 and 12-2 |
| 83 | 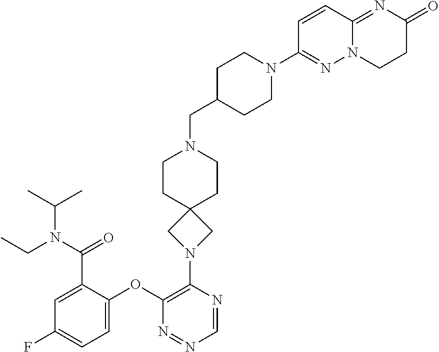 [SMILES: CCN(C(=O)c1cc(F)ccc1Oc1nncnc1N1CC2(CCN(CC3CCN(C4=NN5CCC(=O)N=C5C=C4)CC3)CC2)C1)C(C)C] | 673.4 | ¹H NMR (400 MHz, CD₃OD) δ 8.39- 8.36 (m, 1H), 7.65-7.62 (m, 1H), 7.44- 7.41 (m, 1H), 7.29-7.26 (m, 1H), 7.22- 7.19 (m, 1H), 7.15-7.12 (m, 1H), 4.40- 4.33 (m, 4H), 4.17-4.14 (m, 2H), 3.94- 3.90 (m, 2H), 3.86-3.77 (m, 1H), 3.56- 3.42 (m, 1H), 3.27-3.24 (m, 1H), 2.93- 2.90 (m, 2H), 2.77-2.74 (m, 2H), 2.44- 2.38 (m, 4H), 2.23-2.20 (m, 2H), 1.89- 1.86 (m, 8H), 1.22-1.04 (m, 9H), 0.82- 0.77 (m, 2H). | 7-1 |
| 84 | 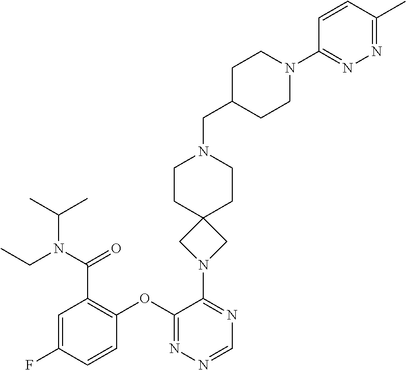 [SMILES: CCN(C(=O)c1cc(F)ccc1Oc1nncnc1N1CC2(CCN(CC3CCN(c4ccc(C)nn4)CC3)CC2)C1)C(C)C] | 618.4 | ¹H NMR (400 MHz, CD₃OD) δ 8.39- 8.36 (m, 1H), 8.29 (d, J = 1.5 Hz, 1H), 7.44-7.36 (m, 1H), 7.31-7.16 (m, 2H), 7.07 (s, 1H), 4.40-4.32 (m, 4H), 3.97- 3.85 (m, 2H), 3.84-3.75 (m, 1H), 3.56- 3.44 (m, 1H), 3.27-3.15 (m, 1H), 2.92 (t, J = 11.8 Hz, 2H), 2.59-2.30 (m, 4H), 2.26 (s, 3H), 2.21 (d, J = 6.5 Hz, 2H), 1.89-1.85 (m, 6H), 1.31-1.23 (m, 1H), 1.22-1.17 (m, 3H), 1.16-1.10 (m, 5H), 1.05 (t, J = 7.1 Hz, 1H), 0.80 (d, J = 6.3 Hz, 2H). | 7-1 and 19-2 |
| 85 | 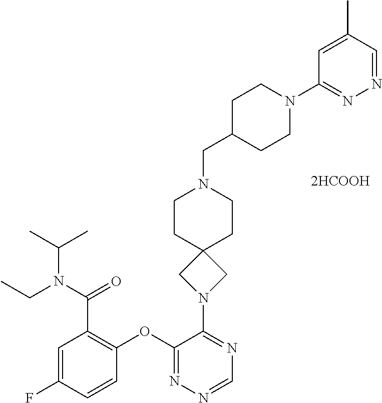 [SMILES: CCN(C(=O)c1cc(F)ccc1Oc1nncnc1N1CC2(CCN(CC3CCN(c4cc(C)cnn4)CC3)CC2)C1)C(C)C ; "2HCOOH"] | 618.4 | ¹H NMR (400 MHz, CD₃OD) δ 8.42 (s, 1H), 8.28 (s, 2H), 7.43-7.37(m, 1H), 7.34-7.31 (m, 1H), 7.30-7.20 (m, 3H), 4.52-4.43 (m, 2H), 4.42-4.31 (m, 2H), 4.10-3.99 (m, 2H), 3.87-3.76 (m, 1H), 3.52-3.40 (m, 1H), 3.28-3.15 (m, 4H), 3.04-2.92 (m, 4H), 2.47 (s, 3H), 2.24- 2.07 (m, 5H), 1.93-1.82 (m, 2H), 1.45- 1.20 (m, 3H), 1.20-1.02 (m, 7H), 0.79 (d, J = 5.8 Hz, 2H). | 7-1 and 19-3 |
| 86 | 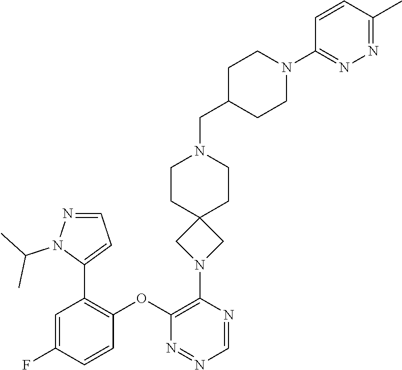 [SMILES: Cc1ccc(N2CCC(CN3CCC4(CC3)CN(c3ncnnc3Oc3ccc(F)cc3-c3ccnn3C(C)C)C4)CC2)nn1] | 613.4 | ¹H NMR (400 MHz, CD₃OD) δ 8.32 (s, 1H), 8.30 (s, 1H), 7.53 (s, 1H), 7.49-7.46 (m, 1H), 7.34 (t, J = 7.0 Hz, 1H), 7.25- 7.18 (m, 1H), 7.08 (s, 1H), 6.19 (s, 1H), 4.43-4.25 (m, 3H), 3.96 (s, 2H), 3.83 (s, 2H), 2.92 (t, J = 12.5 Hz, 2H), 2.55- 2.28 (br, 4H), 2.26 (s, 3H), 2.19 (d, J = 6.4 Hz, 2H), 1.89-1.83 (m, 3H), 1.82- 1.77 (m, 4H), 1.30 (d, J = 6.5 Hz, 6H), 1.22-1.16 (m, 2H). | 7-12 and 19-2 |
| 87 | 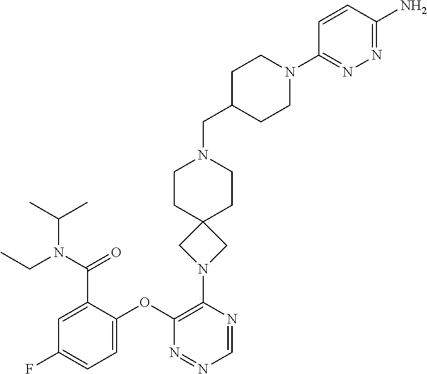 [SMILES: CCN(C(=O)c1cc(F)ccc1Oc1nncnc1N1CC2(CCN(CC3CCN(c4ccc(N)nn4)CC3)CC2)C1)C(C)C] | 619.6 | ¹H NMR (400 MHz, a mixture of CDCl₃ and CD₃OD) δ 8.40 (s, 1H), 7.55-7.52 (m, 1H), 7.32-7.29 (m, 1H), 7.20-7.17 (m, 2H), 7.08-7.05 (m, 1H), 4.44-4.40 (m, 4H), 4.02-4.37 (m, 4H), 3.83-3.79 (m, 1H), 3.46-3.43 (m, 1H), 3.22-3.14 (m, 1H), 2.89-2.85 (m, 4H), 2.68-2.64 (m, 2H), 2.08-2.02 (m, 5H), 1.86-1.82 (m, 2H), 1.27-1.04 (m, 9H), 0.80-0.75 (m, 2H). | 7-1 and 22 |
| 88 | 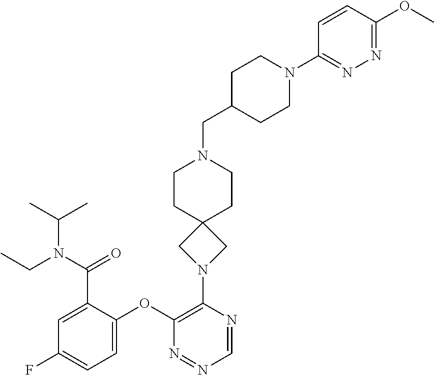 [SMILES: CCN(C(=O)c1cc(F)ccc1Oc1nncnc1N1CC2(CCN(CC3CCN(c4ccc(OC)nn4)CC3)CC2)C1)C(C)C] | 634.4 | ¹H NMR (400 MHz, CD₃OD) δ 8.35 (s,1H), 7.39-7.36 (m, 1H), 7.28-7.25 (m, 1H), 7.23-7.13 (m, 2H), 6.92 (d, J = 9.7 Hz, 1H), 4.40-4.21 (m, 2H), 4.11 (d, J = 12.8 Hz, 2H), 3.94 (s, 3H), 3.93-3.76 (m, 2H), 3.45-3.35 (m,1H), 3.16-3.08 (m,1H), 2.91-2.85 (m,2H), 2.49-2.35 (br, 4H), 2.21 (d, J = 6.7 Hz, 2H), 1.90-1.77 (m, 7H), 1.29-1.13 (m, 7H), 1.08-1.02 (m, 2H), 0.90-0.80 (m, 2H). | 7-1 and 24-1 |
| 89 | 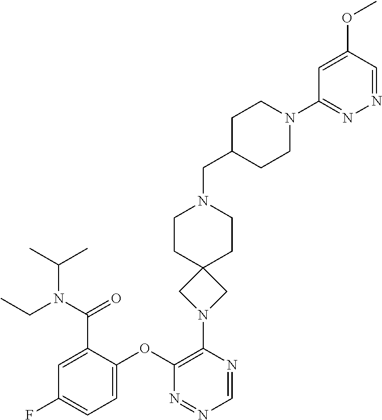 [SMILES: CCN(C(=O)c1cc(F)ccc1Oc1nncnc1N1CC2(CCN(CC3CCN(c4cc(OC)cnn4)CC3)CC2)C1)C(C)C] | 634.4 | ¹H NMR (400 MHz, CD₃OD) δ 8.29 (s, 1H), 8.08 (s, 1H), 7.32 (dd, J = 8.9, 4.6 Hz, 1H), 7.18 (t, J = 8.6 Hz, 1H), 7.11 (d, J = 7.9 Hz, 1H), 6.52 (s, 1H), 4.27 (d, J = 13.2 Hz, 4H), 3.82 (s, 2H), 3.79 (s, 3H), 3.38 (s, 1H), 3.15 (s, 1H), 2.86 (t, J = 12.5 Hz, 2H), 2.36 (s, 4H), 2.11-1.95 (m, 2H), 1.78 (d, J = 5.6 Hz, 8H), 1.15- 1.07 (m, 6H), 0.97 (t, J = 7.0 Hz, 3H), 0.80 (t, J = 6.6 Hz, 2H). | 7-1 and 24-2 |
| 90 | 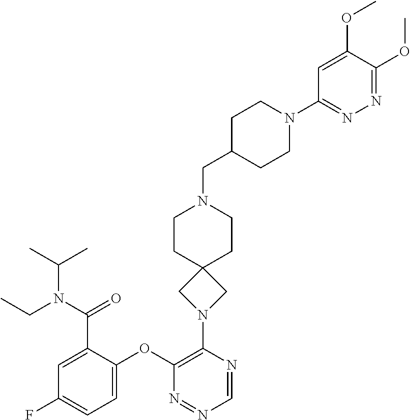 [SMILES: CCN(C(=O)c1cc(F)ccc1Oc1nncnc1N1CC2(CCN(CC3CCN(c4cc(OC)c(OC)nn4)CC3)CC2)C1)C(C)C] | 664.4 | ¹H NMR (400 MHz, CD₃OD) δ 8.38 (s, 1H), 7.43-7.40 (m, 1H), 7.33-7.23 (m, 1H), 7.21-7.18 (m, 1H), 6.68 (s, 1H), 4.39-4.36 (m, 2H), 4.17-4.06 (m, 2H), 3.95 (s, 3H), 3.94-3.89 (m, 2H), 3.88 (s, 3H), 3.83-3.80 (m, 1H), 3.26-3.14 (m, 1H), 2.88-2.85 (m, 2H), 2.46-2.41 (m, 3H), 2.24-2.20 (m, 2H), 1.88-1.83 (m, 7H), 1.37-1.00 (m, 11H), 0.83- 0.79 (m, 2H). | 7-1 and 24-3 |
| 91 | 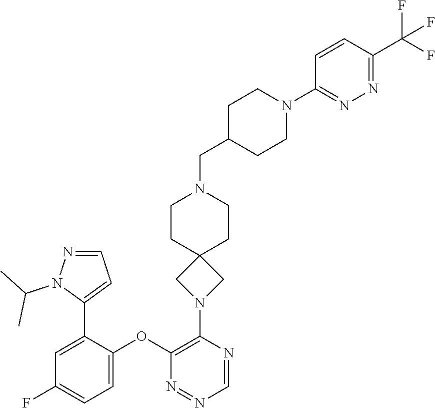 [SMILES: CC(C)n1nccc1-c1cc(F)ccc1Oc1nncnc1N1CC2(CCN(CC3CCN(c4ccc(C(F)(F)F)nn4)CC3)CC2)C1] | 667.4 | ¹H NMR (400 MHz, CD₃OD) δ 8.33 (s, 1H), 7.63 (d, J = 9.7 Hz, 1H), 7.54 (d, J = 1.8 Hz, 1H), 7.51-7.47 (m, 1H), 7.39- 7.29 (m, 2H), 7.24-7.21 (m, 1H), 6.21- 6.19 (m, 1H), 4.53 (d, J = 13.6 Hz, 2H), 4.35-4.29 (m, 1H), 3.97 (s, 2H), 3.84 (s, 2H), 3.06 (t, J = 12.1 Hz, 2H), 2.57-2.28 (br, 4H), 2.21 (d, J = 6.6 Hz, 2H), 1.95- 1.87 (m, 3H), 1.85-1.76 (m, 4H), 1.31 (d, J = 6.6 Hz, 6H), 1.26-1.15 (m, 2H). | 7-12 and 12-3 |
| 92 | 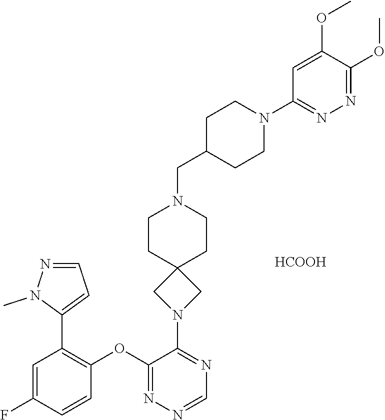 | 631.4 | ¹H NMR (400 MHz, CD₃OD) δ 8.33 (s, 1H), 7.51-7.42 (m, 2H), 7.39-7.27 (m, 2H), 6.76 (s, 1H), 6.25 (d, J = 1.9 Hz, 1H), 424-4.138 (m, 4H), 3.99-3.93 (m, 5H), 3.92 (s, 3H), 3.69 (s, 3H), 3.22-3.02 (m, 4H), 3.01-2.92 (m, 2H), 2.88 (d, J = 6.8 Hz, 2H), 2.21-2.00 (m, 6H), 1.95- 1.85 (m, 2H), 1.43-1.30 (m, 2H). | 7-11 and 24-3 |
| 93 | 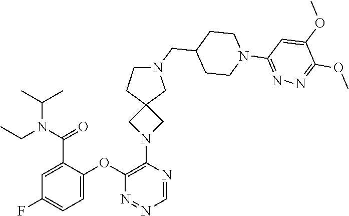 [SMILES: CCN(C(=O)c1cc(F)ccc1Oc1nncnc1N1CC2(CCN(CC3CCN(c4cc(OC)c(OC)nn4)CC3)C2)C1)C(C)C] | 650.4 | ¹H NMR (400 MHz, DMSO-d6) δ 8.44 (s, 1H), 7.44-7.40 (m, 1H), 7.33 (d, J = 7.6 Hz, 2H), 6.71 (s, 1H), 4.41 (s, 2H), 4.14 (d, J = 12.6 Hz, 2H), 4.03 (s, 2H), 3.84 (s, 3H), 3.81 (s, 3H), 3.59 (dt, J = 13.0, 6.5 Hz, 1H), 3.09 (s, 1H), 2.99 (s, 1H), 2.73 (t, J = 11.6 Hz, 4H), 2.64 (s, 1H), 2.53 (s, 1H), 2.25 (d, J = 7.0 Hz, 2H), 2.03 (s, 2H), 1.75 (d, J = 11.6 Hz, 2H), 1.63 (s, 1H), 1.12-1.03 (m, 6H), 1.02-0.91 (m, 3H), 0.69 (s, 2H). | 7-17 and 24-3 |
| 94 | 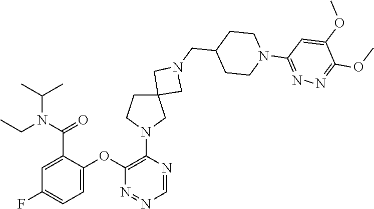 [SMILES: CCN(C(=O)c1cc(F)ccc1Oc1nncnc1N1CCC2(CN(CC3CCN(c4cc(OC)c(OC)nn4)CC3)C2)C1)C(C)C] | 650.4 | ¹H NMR (400 MHz, CD₃OD) δ 8.42 (s, 1H), 7.40 (s, 1H), 7.32-7.17 (m, 2H), 6.68 (s, 1H), 4.20-4.06 (m, 4H), 3.95 (s, 3H), 3.88 (s, 3H), 3.84-3.76 (m, 2H), 3.74-3.63 (m, 1H), 3.55-3.42 (m, 1H), 3.33 (d, J = 6.8 Hz, 2H), 3.24-3.14 (m, 1H), 2.83 (t, J = 12.4 Hz, 2H), 2.48-2.43 (m, 2H), 2.28-2.15 (m, 2H), 1.81 (d, J = 12.8 Hz, 2H), 1.63 (s, 1H), 1.34-1.22 (m, 3H), 1.18-1.12 (m, 4H), 1.08 (t, J = 6.9 Hz, 3H), 0.83-0.75 (m, 2H). | 7-16 and 24-3 |
| 95 | 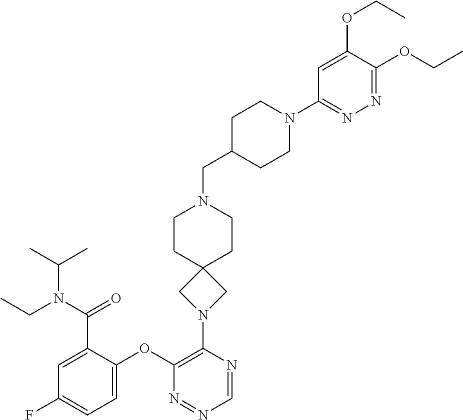 [SMILES: CCOc1cc(N2CCC(CN3CCC4(CC3)CN(c3ncnnc3Oc3ccc(F)cc3C(=O)N(CC)C(C)C)C4)CC2)nnc1OCC] | 692.4 | ¹H NMR (400 MHz, CD₃OD) δ 8.41 (s, 1H), 8.23 (s, 1H), 7.42-7.39 (m, 1H), 7.34-7.15 (m, 2H), 6.82 (s, 1H), 4.46 (s, 2H), 4.35 (q, J = 7.1 Hz, 2H), 4.21 (q, J = 7.0 Hz, 2H), 4.17-4.10 (m, 2H), 4.04-4.00 (m, 2H), 3.86-3.76 (m, 1H), 3.52-3.43 (m, 1H), 3.27-3.17 (m, 3H), 3.11 (t, J = 12.3 Hz, 3H), 3.00 (d, J = 6.6 Hz, 2H), 2.24-2.12 (m, 5H), 1.7-1.90 (m, 2H), 1.7-1.38 (m, 8H), 1.17-1.05 (m, 7H), 0.85-0.77 (m, 2H), 0.71-0.03(m, 2H). | 7-1 and 24-12 |
| 96 | 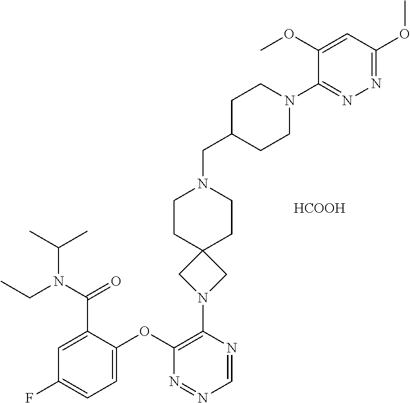 [SMILES: CCN(C(=O)c1cc(F)ccc1Oc1nncnc1N1CC2(CCN(CC3CCN(c4nnc(OC)cc4OC)CC3)CC2)C1)C(C)C ; "HCOOH"] | 664.4 | ¹H NMR (400 MHz, CD₃OD) δ 8.42 (s, 1H), 8.38 (s, 1H), 7.42-7.39 (m, 1H), 7.32-7.27 (m, 1H), 7.25-7.20 (m, 1H), 6.55 (s, 1H), 4.48-4.45 (m, 2H), 4.05- 4.01 (m, 2H), 3.96 (s, 3H), 3.91 (s, 3H), 3.87-3.81 (m, 2H), 3.49-3.46 (m, 1H), 3.23-3.15 (m, 4H), 2.96-2.92 (m, 2H), 2.88-2.77 (m, 2H), 2.15-2.10 (m, 6H), 1.88-1.85 (m, 2H), 1.48-1.45 (m, 2H), 1.16-1.10 (m, 8H), 0.82-0.77 (m, 2H). | 7-1 and 24-5 |
| 97 | 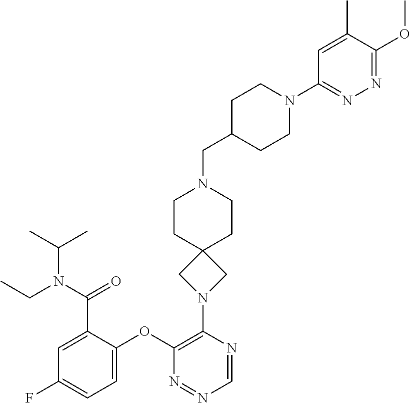 [SMILES: CCN(C(=O)c1cc(F)ccc1Oc1nncnc1N1CC2(CCN(CC3CCN(c4cc(C)c(OC)nn4)CC3)CC2)C1)C(C)C] | 648.4 | ¹H NMR (400 MHz, CD₃OD) δ 8.38 (s, 1H), 7.43-7.40 (m, 1H), 7.29-7.26 (m, 1H), 7.25-7.18 (m, 1H), 7.17 (s, 1H), 4.38-4.35 (m, 2H), 4.14-4.10 (m, 2H), 3.96 (s, 3H), 3.93-3.90 (m, 2H), 3.84- 3.76 (m, 1H), 3.56-3.44 (m, 1H), 3.27- 3.23 (m, 1H), 2.86-2.82 (m, 2H), 2.39- 2.35 (m, 4H), 2.23-2.19 (m, 2H), 2.16 (s, 3H), 1.92-1.79 (m, 7H), 1.25-1.01 (m, 9H), 0.83-0.78 (m, 2H). | 7-1 and 24-6 |
| 98 | 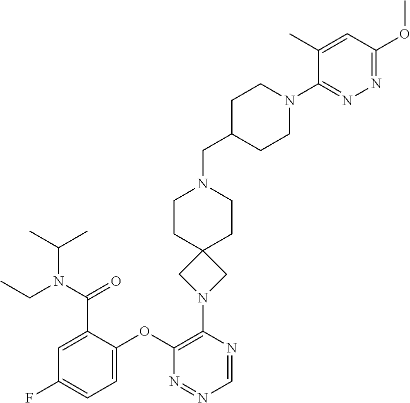 [SMILES: CCN(C(=O)c1cc(F)ccc1Oc1nncnc1N1CC2(CCN(CC3CCN(c4nnc(OC)cc4C)CC3)CC2)C1)C(C)C] | 648.4 | ¹H NMR (400 MHz, CD₃OD) δ 8.38 (s, 1H), 7.48-7.36 (m, 1H), 7.32-7.24 (m, 1H), 7.22-7.19 (m, 1H), 6.92-6.88 (m, 1H), 4.38-4.35 (m, 2H), 3.96 (s, 3H), 3.95-3.92 (m, 2H), 3.86-3.75 (m, 1H), 3.57-3.45 (m, 1H), 3.43-3.40 (m, 2H), 3.27-3.24 (m, 1H), 2.84-2.80 (m, 2H), 2.41-2.38 (m, 4H), 2.29-2.26 (m, 5H), 1.85-1.76 (m, 7H), 1.38-1.34 (m, 2H), 1.25-1.06 (m, 7H), 0.83-0.78 (m, 2H). | 7-1 and 24-7 |
| 99 | 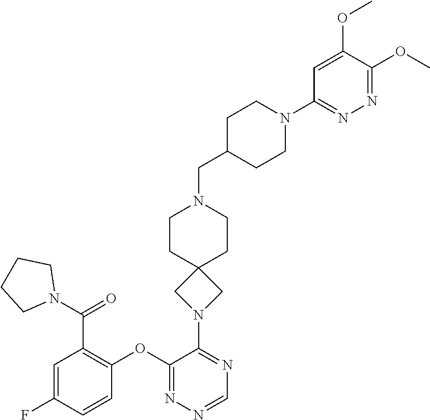 [SMILES: COc1cc(N2CCC(CN3CCC4(CC3)CN(c3ncnnc3Oc3ccc(F)cc3C(=O)N3CCCC3)C4)CC2)nnc1OC] | 648.4 | ¹H NMR (400 MHz, CD₃OD) δ 8.38 (s, 1H), 7.38-7.32 (m, 1H), 7.29-7.21 (m, 2H), 6.72 (s, 1H), 4.52-4.32 (m, 2H), 4.18-4.11 (m, 2H), 4.09-4.00 (m, 1H), 3.96 (s, 3H), 3.92 (s, 3H), 3.35 (t, J = 6.5 Hz, 2H), 3.33-3.31 (m, 2H), 3.10 (s, 4H), 3.20-3.05 (m, 4H), 3.04-2.96 (m, 2H), 2.87-2.81(m, 2H), 2.17-2.11 (m, 4H), 2.10-2.03 (m, 1H), 1.94-1.87 (m, 2H), 1.86-1.74 (m, 4H), 1.45-1.32 (m, 2H), 1.32-1.25 (m, 1H). | 7-3 and 24-3 |
| 100 | 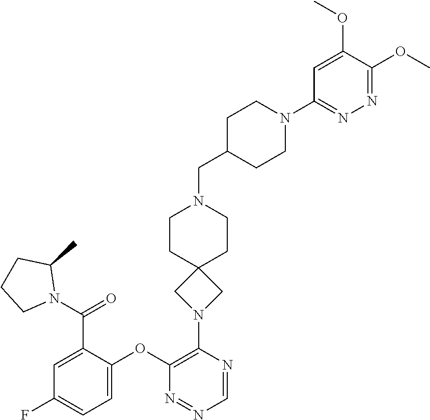 [SMILES: COc1cc(N2CCC(CN3CCC4(CC3)CN(c3ncnnc3Oc3ccc(F)cc3C(=O)N3CCC[C@H]3C)C4)CC2)nnc1OC] | 662.4 | ¹H NMR (400 MHz, DMSO-d6) δ 8.45- 8.42 (m, 1H), 7.43-7.38 (m, 1H), 7.36 (t, J = 7.8 Hz, 2H), 6.71 (s, 1H), 4.23 (q, J = 9.8 Hz, 2H), 4.14 (d, J = 12.9 Hz, 2H), 3.91 (dd, J = 10.6, 7.0 Hz, 1H), 3.84 (s, 3H), 3.81 (s, 3H), 3.18-3.14 (m, 2H), 2.72 (d, J = 11.5 Hz, 2H), 2.31 (s, 4H), 2.08 (d, J = 6.4 Hz, 2H), 1.72 (d, J = 10.5 Hz, 8H), 1.65-1.61 (m, 2H), 1.45-1.41 (m, 1H), 1.21 (s, 3H), 1.08 (d, J = 10.6 Hz, 2H), 0.95 (d, J = 6.3 Hz, 2H). | 7-4 and 24-3 |
| 101 | 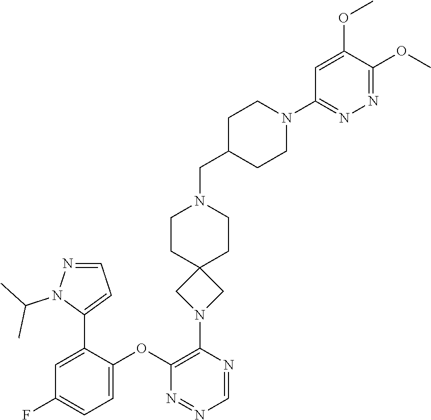 [SMILES: COc1cc(N2CCC(CN3CCC4(CC3)CN(c3ncnnc3Oc3ccc(F)cc3-c3ccnn3C(C)C)C4)CC2)nnc1OC] | 659.4 | ¹H NMR (400 MHz, CD₃OD) δ 8.32 (s, 1H), 7.53 (d, J = 1.8 Hz, 1H), 7.50-7.47 (m, 1H), 7.38-7.30 (m, 1H), 7.23-7.20 (m, 1H), 6.68 (s, 1H), 6.19 (d, J = 1.8 Hz, 1H), 4.34-4.28 (m, 1H), 4.12 (d, J = 13.0 Hz, 2H), 3.96 (s, 2H), 3.94 (s, 3H), 3.87 (s, 3H), 3.83 (s, 2H), 2.85 (t, J = 11.7 Hz, 2H), 2.55-2.22 (br, 4H), 2.20 (d, J = 6.7 Hz, 2H), 1.86-1.83 (m, 3H), 1.82-1.76 (m, 4H), 1.30 (d, J = 6.6 Hz, 6H), 1.28-1.19 (m, 2H). | 7-12 and 24-3 |
| 102 | 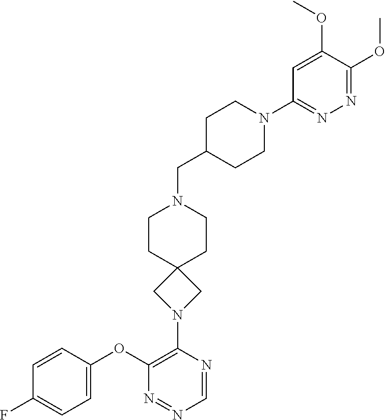 [SMILES: COc1cc(N2CCC(CN3CCC4(CC3)CN(c3ncnnc3Oc3ccc(F)cc3)C4)CC2)nnc1OC] | 551.3 | ¹H NMR (400 MHz, CD₃OD) δ 8.35 (s, 1H), 7.26-7.23 (m, 2H), 7.18-7.14 (m, 2H), 6.67 (s, 1H), 4.39 (s, 2H), 4.14- 4.10 (m, 2H), 3.96-3.91 (m, 5H), 3.87 (s, 3H), 2.86-2.82 (m, 2H), 2.46-2.38 (m, 4H), 2.23-2.18 (m, 2H), 1.89-1.83 (m, 7H), 1.26-1.21 (m, 2H). | 7-7 and 24-3 |
| 103 | 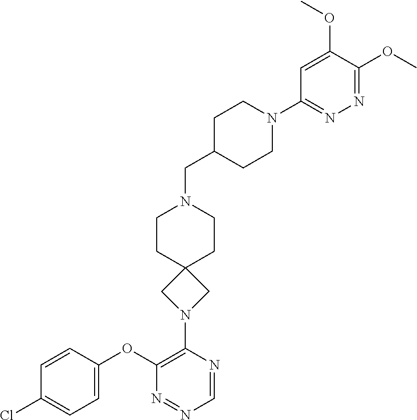 [SMILES: COc1cc(N2CCC(CN3CCC4(CC3)CN(c3ncnnc3Oc3ccc(Cl)cc3)C4)CC2)nnc1OC] | 567.3 | ¹H NMR (400 MHz, CD₃OD) δ 8.27 (d, J = 0.4 Hz, 1H), 7.33 (d, J = 8.6 Hz, 2H), 7.16 (d, J = 8.7 Hz, 2H), 6.59 (s, 1H), 4.30 (s, 2H), 4.04 (d, J = 12.8 Hz, 2H), 3.86 (s, 3H), 3.85 (s, 2H), 3.79 (s, 3H), 2.74 (d, J = 11.6 Hz, 2H), 2.29-2.27(m, 4H), 2.12 (d, J = 6.6 Hz, 2H), 1.80 (d, J = 5.2 Hz, 4H), 1.71 (s, 1H), 1.17 (d, J = 7.3 Hz, 4H). | 7-8 and 24-3 |
| 104 | 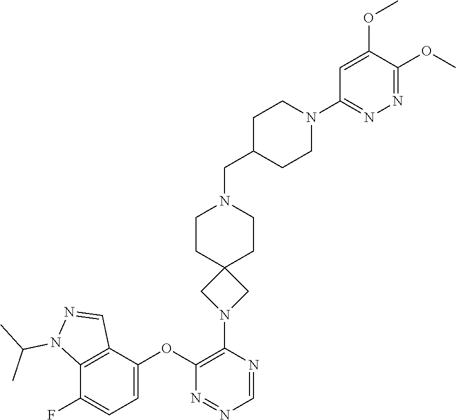 [SMILES: COc1cc(N2CCC(CN3CCC4(CC3)CN(c3ncnnc3Oc3ccc(F)c5c3cnn5C(C)C)C4)CC2)nnc1OC] | 633.3 | ¹H NMR (400 MHz, CD₃OD) δ 8.38 (s, 1H), 7.95 (s, 1H), 7.15-7.09 (m, 1H), 6.94-6.91 (m, 1H), 6.67 (s, 1H), 5.18- 5.12 (m, 1H), 4.45 (s, 2H), 4.12 (d, J = 12.7 Hz, 2H), 3.97 (s, 2H), 3.94 (s, 3H), 3.87 (s, 3H), 2.84 (t, J = 12.1 Hz, 2H), 2.55-2.25 (br, 4H), 2.20 (d, J = 6.6 Hz, 2H), 1.95-1.88 (m, 4H), 1.86-1.78 (m, 3H), 1.56 (d, J = 6.6 Hz, 6H), 1.27-1.18 (m, 2H). | 7-22 and 24-3 |
| 105 | 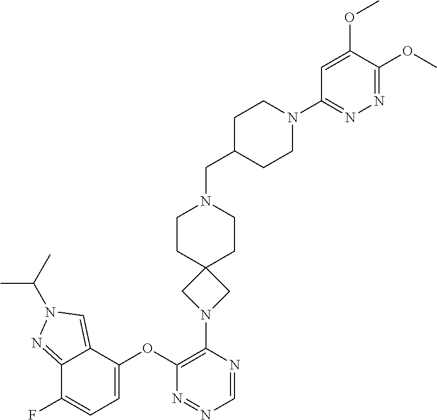 [SMILES: COc1cc(N2CCC(CN3CCC4(CC3)CN(c3ncnnc3Oc3ccc(F)c5nn(C(C)C)cc35)C4)CC2)nnc1OC] | 633.4 | ¹H NMR (400 MHz, CD₃OD) δ 8.38 (s, 1H), 8.34 (d, J = 2.5 Hz, 1H), 7.00-6.95 (m, 1H), 6.85-6.82 (m, 1H), 6.69 (s, 1H), 4.86-4.79 (m, 1H), 4.45 (s, 2H), 4.13 (d, J = 13.1 Hz, 2H), 3.98 (s, 2H), 3.95 (s, 3H), 3.88 (s, 3H), 2.85 (t, J = 11.7 Hz, 2H), 2.55-2.30 (br, 4H), 2.22 (d, J = 6.7 Hz, 2H), 1.95-1.89 (m, 4H), 1.89-1.77 (m, 3H), 1.61 (d, J = 6.7 Hz, 6H), 1.29- 1.21 (m, 2H). | 7-21 and 24-3 |
| 106 | 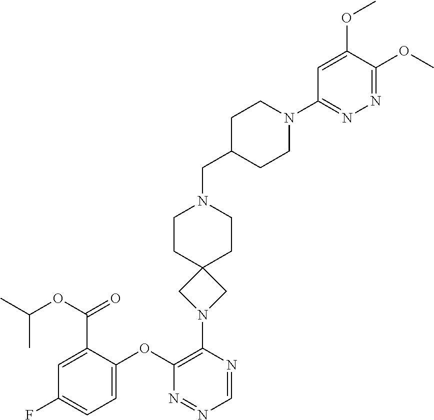 [SMILES: COc1cc(N2CCC(CN3CCC4(CC3)CN(c3ncnnc3Oc3ccc(F)cc3C(=O)OC(C)C)C4)CC2)nnc1OC] | 637.4 | ¹H NMR (400 MHz, CD₃OD) δ 8.34 (s, 1H), 7.74-7.71 (m, 1H), 7.47-7.40 (m, 1H), 7.37-7.33 (m, 1H), 6.68 (s, 1H), 5.13-5.01 (m, 1H), 4.44 (s, 2H), 4.13 (d, J = 13.0 Hz, 2H), 3.98 (s, 2H), 3.95 (s, 3H), 3.88 (s, 3H), 2.85 (t, J = 11.6 Hz, 2H), 2.55-2.32 (m, 4H), 2.22 (d, J = 6.7 Hz, 2H), 1.93-1.89 (m, 4H), 1.87- 1.80 (m, 3H), 1.28-1.23 (m, 2H), 1.17 (J = 6.3 Hz, 6H). | 7-10 and 24-3 |
| 107 | 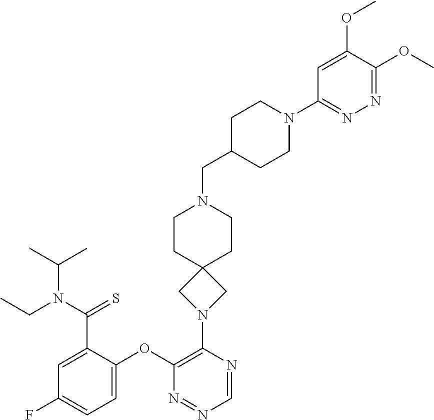 [SMILES: CCN(C(=S)c1cc(F)ccc1Oc1nncnc1N1CC2(CCN(CC3CCN(c4cc(OC)c(OC)nn4)CC3)CC2)C1)C(C)C] | 680.4 | ¹H NMR (400 MHz, CD₃OD) δ 8.37 (s, 1H), 7.35-7.32 (m, 1H), 7.19-6.99 (m, 2H), 6.68 (s, 1H), 4.57 (d, J = 10.1 Hz, 1H), 4.31-4.26 (m, 1H), 4.21-4.09 (m, 3H), 3.95 (s, 3H), 3.94-3.89 (m, 1H), 3.88 (s, 3H), 3.70-3.54 (m, 1H), 3.30- 3.21 (m, 1H), 2.85 (t, J = 12.0 Hz, 2H), 2.58-2.27 (m, 4H), 2.21 (d, J = 6.5 Hz, 2H), 1.92-1.74 (m, 7H), 1.36-1.04 (m, 10H), 0.82 (d, J = 6.5 Hz, 2H). | 7-23 and 24-3 |
| 108 | 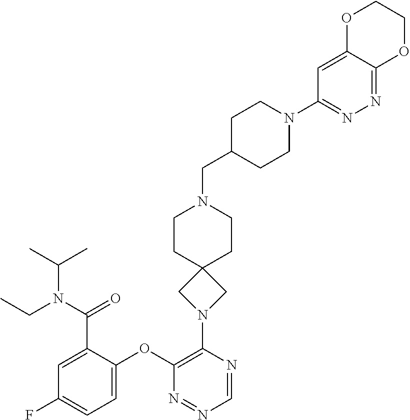 [SMILES: CCN(C(=O)c1cc(F)ccc1Oc1nncnc1N1CC2(CCN(CC3CCN(c4cc5c(nn4)OCCO5)CC3)CC2)C1)C(C)C] | 662.6 | ¹H NMR (400 MHz, CD;OD) δ 8.38 (s, 1H), 7.44-7.40 (m, 1H), 7.26-7.23 (m, 2H), 6.77 (s, 1H), 4.47-4.30 (m, 6H), 4.14-4.10 (m, 2H), 3.94-3.90 (m, 2H), 3.85-3.74 (m, 1H), 3.50-3.47 (m, 1H), 3.25-3.23 (m, 1H), 2.86-2.83 (m, 2H), 2.47-2.44 (m, 4H), 2.23-2.20 (m, 2H), 1.88-1.83 (m, 7H), 1.26-1.05 (m, 9H), 0.83-0.77 (m, 2H). | 7-1 and 24-8 |
| 109 | 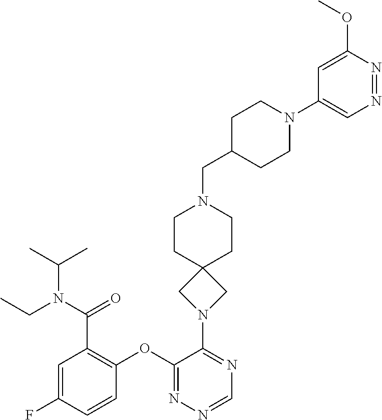 [SMILES: CCN(C(=O)c1cc(F)ccc1Oc1nncnc1N1CC2(CCN(CC3CCN(c4cnnc(OC)c4)CC3)CC2)C1)C(C)C] | 634.4 | ¹H NMR (400 MHz, CD₃OD) δ 8.57 (d, J = 2.6 Hz, 1H), 8.39-8.33 (m, 1H), 7.40 (dt, J = 9.4, 4.8 Hz, 1H), 7.33-7.16 (m, 2H), 6.28 (d, J = 2.5 Hz, 1H), 4.42- 4.28 (m, 2H), 4.02-3.98(m, 1H), 3.98- 3.96 (m, 1H), 3.96 (s, 3H), 3.95-3.87 (m, 2H), 3.84-3.75 (m, 1H), 3.56-3.42 (m, 1H), 3.27-3.15 (m, 1H), 2.93 (t, J = 11.7 Hz, 2H), 2.65-2.25 (m, 4H), 2.21 (d, J = 6.5 Hz, 2H), 1.89-1.84 (m, 6H), 1.30-1.23 (m, 1H), 1.22-1.17 (m, 3H), 1.16-1.10 (m, 5H), 1.05 (t, J = 7.1 Hz, 1H), 0.79 (d, J = 6.4 Hz, 2H). | 7-1 and 21 |
| 110 | 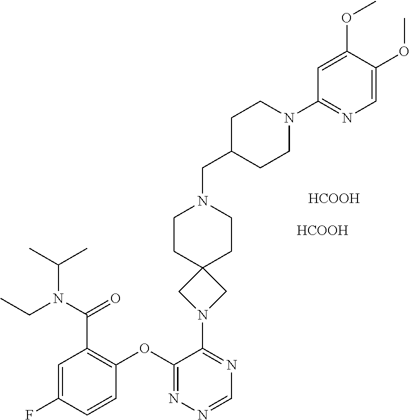 [SMILES: CCN(C(=O)c1cc(F)ccc1Oc1nncnc1N1CC2(CCN(CC3CCN(c4cc(OC)c(OC)cn4)CC3)CC2)C1)C(C)C ; "HCOOH" ; "HCOOH"] | 663.4 | ¹H NMR (400 MHz, CD₃OD) δ 8.42 (s, 1H), 8.30 (s, 2H), 7.59 (s, 1H), 7.45-7.37 (m, 1H), 7.32-7.20 (m, 2H), 6.46 (s, 1H), 4.51-4.41 (m, 2H), 4.17-4.10 (m, 2H), 4.09-3.97 (m, 2H), 3.90 (s, 3H), 3.85-3.80 (m, 1H), 3.78 (s, 3H), 3.55- 3.43 (m, 1H), 3.27-3.15 (m, 4H), 2.96- 2.86 (m, 4H), 2.18-2.11 (m, 4H), 1.92- 1.83 (m, 2H), 1.43-1.25 (m, 4H), 1.21- 1.11 (m, 6H), 1.07 (t, J = 7.1 Hz, 1H), 0.80 (d, J = 5.6 Hz, 2H). | 7-1 and 24-10 |
| 111 | 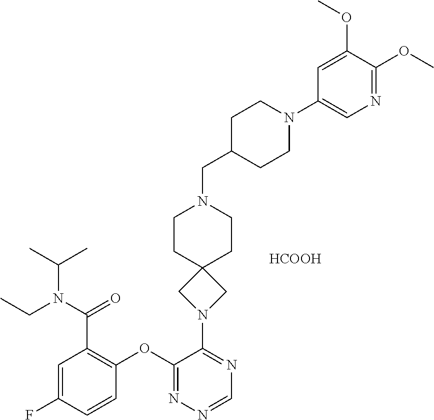 [SMILES: CCN(C(=O)c1cc(F)ccc1Oc1nncnc1N1CC2(CCN(CC3CCN(c4cnc(OC)c(OC)c4)CC3)CC2)C1)C(C)C ; "HCOOH"] | 663.4 | ¹H NMR (400 MHz, CD₃OD) δ 8.42 (s, 1H), 8.37 (s, 1H), 7.42-7.37 (m, 1H), 7.32-7.20 (m, 3H), 7.01 (d, J = 2.2 Hz, 1H), 4.51-4.40 (m, 2H), 4.07-3.94 (m, 2H), 3.86 (s, 3H), 3.81 (s, 3H), 3.58-3.52 (m, 2H), 3.51-3.42 (m, 1H), 3.26-3.08 (m, 4H), 2.93 (d, J = 6.3 Hz, 2H), 2.70 (t, J = 11.6 Hz, 2H), 2.20-2.09 (m, 4H), 1.93-1.87 (m, 2H), 1.52-1.38 (m, 2H), 1.33-1.27 (m, 3H), 1.21-1.10 (m, 6H), 1.07 (t, J = 7.1 Hz, 1H), 0.79 (d, J = 5.9 Hz, 2H). | 7-1 and 24-11 |
| 112 | 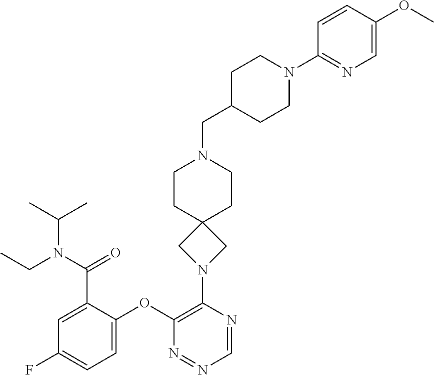 [SMILES: CCN(C(=O)c1cc(F)ccc1Oc1nncnc1N1CC2(CCN(CC3CCN(c4ccc(OC)cn4)CC3)CC2)C1)C(C)C] | 633.4 | ¹H NMR (400 MHz, CD₃OD) δ 8.38 (S, 1H), 7.80 (d, J = 3.1 Hz, 1H), 7.44-7.37 (m, 1H), 7.31-7.19 (m, 3H), 6.81 (d, J = 9.2 Hz, 1H), 4.45-4.30 (m, 2H), 4.08- 4.02 (m, 2H), 3.97-3.88 (m, 2H), 3.84- 3.77 (m, 1H), 3.77 (s, 3H), 3.57-3.44 (m, 1H), 3.28-3.19 (m, 1H), 2.80-2.71 (m, 2H), 2.65-2.42( m, 3H), 2.36-2.36 (m, 2H), 1.95-1.87 (m, 4H), 1.86-1.77 (m, 3H), 1.31-1.24 (m, 2H), 1.23-1.17 (m, 2H), 1.17-1.11 (m, 5H), 1.06 (t, J = 7.1 Hz, 1H), 0.81 (d, J = 6.2 Hz, 2H). | 7-1 and 38 |
| 113 | 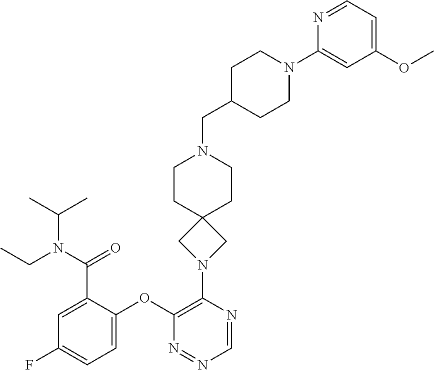 [SMILES: CCN(C(=O)c1cc(F)ccc1Oc1nncnc1N1CC2(CCN(CC3CCN(c4cc(OC)ccn4)CC3)CC2)C1)C(C)C] | 633.4 | ¹H NMR (400 MHz, CD₃OD) δ 8.27 (s, 1H), 7.78 (d, J = 5.9 Hz, 1H), 7.29 (dd, J = 9.1, 4.4 Hz, 1H), 7.15 (td, J = 8.6, 2.9 Hz, 1H), 7.08-7.03 (m, 1H), 6.17 (dd, J = 5.9, 2.1 Hz, 1H), 6.13 (d, J = 2.0 Hz, 1H), 4.25 (s, 2H), 4.06 (d, J = 13.0 Hz, 2H), 3.82-3.79 (m, 2H), 3.71 (s, 3H), 2.74 (t, J = 11.5 Hz, 2H), 2.33 (s, 4H), 2.13-2.08 (m, 3H), 1.96-1.89 (m, 1H), 1.79-1.69 (m, 8H), 1.15-1.02 (m, 9H), 0.96 (s, 2H). | 7-1 and 26 |
| 114 | 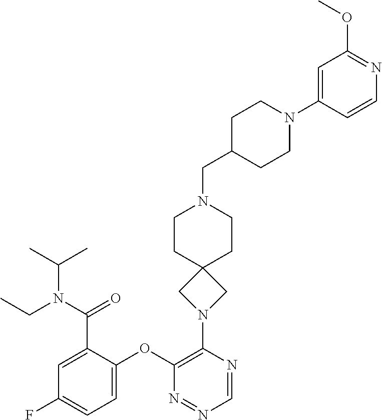 [SMILES: CCN(C(=O)c1cc(F)ccc1Oc1nncnc1N1CC2(CCN(CC3CCN(c4ccnc(OC)c4)CC3)CC2)C1)C(C)C] | 633.4 | ¹H NMR (400 MHz, DMSO-d6) δ 8.42 (d, J = 0.7 Hz, 1H), 7.72 (d, J = 6.1 Hz, 1H), 7.40 (d, J = 4.0 Hz, 1H), 7.34-7.30 (m, 2H), 6.49 (dd, J = 6.2, 1.9 Hz, 1H), 6.04 (d, J = 1.9 Hz, 1H), 4.18 (s, 2H), 3.81 (d, J = 7.6 Hz, 2H), 3.78 (s, 3H), 3.71 (s, 3H), 3.06-3.01 (m, 1H), 2.72 (d, J = 12.0 Hz, 2H), 2.30 (s, 4H), 2.05 (d, J = 6.5 Hz, 2H), 1.68 (d, J = 11.8 Hz, 8H), 1.08-1.04 (m, 6H), 0.99-0.90 (m, 3H), 0.68 (d, J = 6.1 Hz, 2H). | 7-1 and 19-1 |
| 115 | 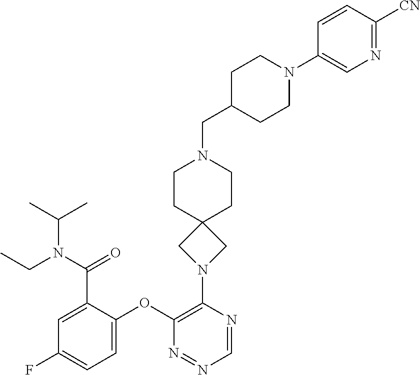 [SMILES: CCN(C(=O)c1cc(F)ccc1Oc1nncnc1N1CC2(CCN(CC3CCN(c4ccc(C#N)nc4)CC3)CC2)C1)C(C)C] | 628.4 | ¹H NMR (400 MHz, DMSO-d6) δ 8.43 (t, J = 1.7 Hz, 1H), 8.37 (d, J = 2.9 Hz, 1H), 7.68 (d, J = 8.9 Hz, 1H), 7.44-7.40 (m, 1H), 7.32 (dd, J = 8.0, 6.2 Hz, 3H), 4.19 (s, 2H), 3.96 (d, J = 13.2 Hz, 2H), 3.78 (s, 2H), 3.59 (dd, J = 13.3, 6.5 Hz, 1H), 3.06-3.01 (m, 1H), 2.86 (d, J = 12.1 Hz, 2H), 2.31 (s, 4H), 2.07 (d, J = 6.6 Hz, 2H), 1.74 (d, J = 12.5 Hz, 8H), 1.11-0.98 (m, 9H), 0.69 (d, J = 6.0 Hz, 2H). | 7-1 and 18-1 |
| 116 | 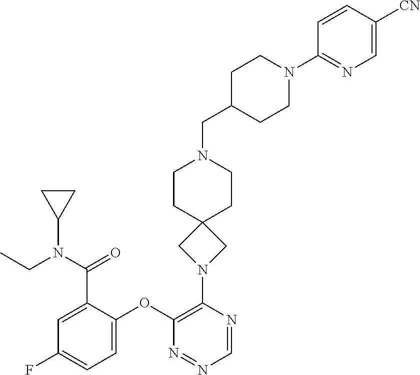 [SMILES: CCN(C(=O)c1cc(F)ccc1Oc1nncnc1N1CC2(CCN(CC3CCN(c4ccc(C#N)cn4)CC3)CC2)C1)C1CC1] | 626.4 | ¹H NMR (400 MHz, CD₃OD) δ 8.44- 8.31 (m, 2H), 7.67-7.64 (m, 1H), 7.43- 7.35 (m, 1H), 7.35-7.16 (m, 2H), 6.82 (d, J = 9.2 Hz, 1H), 4.47 (d, J = 13.4 Hz, 2H), 4.37-4.28 (m, 2H), 3.92 (s, 2H), 3.48 (q, J = 7.0 Hz, 2H), 2.99-2.93 (m, 2H), 2.79-2.62 (m, 1H), 2.55-2.30 (br, 3H), 2.20 (d, J = 6.7 Hz, 2H), 1.95-1.80 (m, 7H), 1.20-1.08 (m, 5H), 0.89-0.42 (m, 4H). | 7-2 and 12-6 |
| 117 | 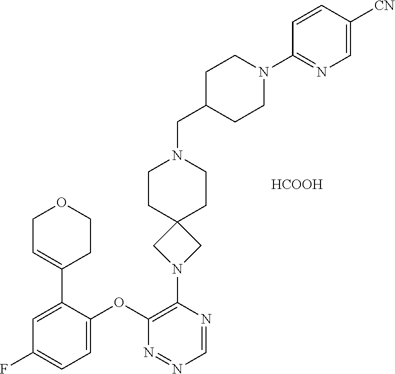 [SMILES: N#Cc1ccc(N2CCC(CN3CCC4(CC3)CN(c3ncnnc3Oc3ccc(F)cc3C3=CCOCC3)C4)CC2)nc1 ; "HCOOH"] | 597.4 | ¹H NMR (400 MHz, CD₃OD) δ 8.53- 8.27 (m, 3H), 7.69-7.66 (m, 1H), 7.24- 7.22 (m, 1H), 7.09-7.06 (m, 2H), 6.85- 6.82 (m, 1H), 5.82-5.79 (m, 1H), 4.53- 4.50 (m, 4H), 4.10-4.05 (m, 4H), 3.72 (s, 2H), 3.18-3.13 (m, 4H), 3.06-2.96 (m, 2H), 2.89-2.86 (m, 2H), 2.31-2.27 (m, 2H), 2.16-2.13 (m, 5H), 1.91-1.88 (m, 2H), 1.28-1.23 (m, 2H). | 7-14 and 12-6 |
| 118 | 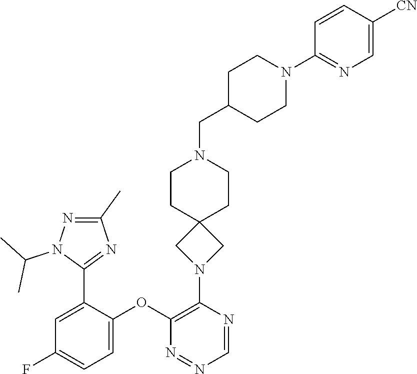 [SMILES: Cc1nc(-c2cc(F)ccc2Oc2nncnc2N2CC3(CCN(CC4CCN(c5ccc(C#N)cn5)CC4)CC3)C2)n(C(C)C)n1] | 638.4 | ¹H NMR (400 MHz, CD₃OD) δ 8.38- 8.35 (m, 1H), 8.21 (s, 1H), 7.71-7.68 (m, 1H), 7.41-7.38 (m, 2H), 7.33-7.30 (m, 1H), 6.86-6.83 (m, 1H), 4.55-4.51 (m, 2H), 4.47-4.38 (m, 1H), 4.06 (s, 2H), 3.86 (s, 2H), 3.18-3.12 (m, 4H), 3.02-2.97 (m, 2H), 2.92-2.88 (m, 2H), 2.30 (s, 3H), 2.13-2.06 (m, 5H), 1.91- 1.87 (m, 2H), 1.37 (d, J = 6.6 Hz, 6H), 1.30-1.25 (m, 2H). | 7-13 and 12-6 |
| 119 | 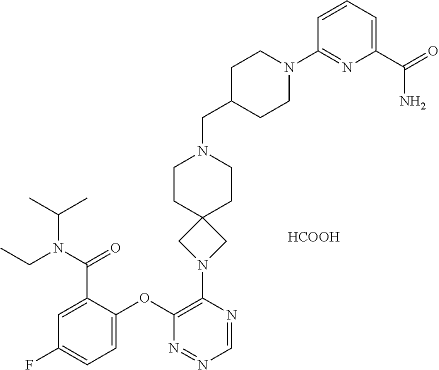 [SMILES: CCN(C(=O)c1cc(F)ccc1Oc1nncnc1N1CC2(CCN(CC3CCN(c4cccc(C(N)=O)n4)CC3)CC2)C1)C(C)C ; "HCOOH"] | 646.4 | ¹H NMR (400 MHz, CD₃OD) δ 8.38 (s, 1H), 7.43-7.40 (m, 1H), 7.33-7.23 (m, 1H), 7.21-7.18 (m, 1H), 6.68 (s, 1H), 4.39-4.36 (m, 2H), 4.17-4.06 (m, 2H), 3.95 (s, 3H), 3.94-3.89 (m, 2H), 3.88 (s, 3H), 3.83-3.80 (m, 1H), 3.26-3.14 (m, 1H), 2.88-2.85 (m, 2H), 2.46-2.41 (m, 3H), 2.24-2.20 (m, 2H), 1.88-1.83 (m, 7H), 1.37-1.00 (m, 11H), 0.83- 0.79 (m, 2H). | 7-1 and 25 |
| 120 | 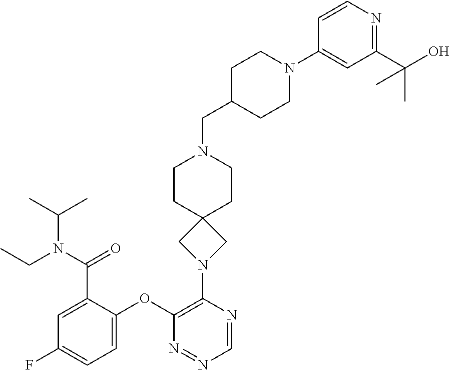 [SMILES: CCN(C(=O)c1cc(F)ccc1Oc1nncnc1N1CC2(CCN(CC3CCN(c4ccnc(C(C)(C)O)c4)CC3)CC2)C1)C(C)C] | 661.4 | ¹H NMR (400 MHz, CD₃OD) δ 8.37 (d, J = 0.7 Hz, 1H), 8.01 (d, J = 6.1 Hz, 1H), 7.40 (dt, J = 9.4, 4.7 Hz, 1H), 7.31-7.24 (m, 1H), 7.20 (dd, J = 8.0, 3.0 Hz, 1H), 7.10 (d, J = 2.5 Hz, 1H), 6.66 (dd, J = 6.1, 2.5 Hz, 1H), 4.36 (d, J = 13.7 Hz, 2H), 4.00 (d, J = 12.8 Hz, 2H), 3.92 (d, J = 8.4 Hz, 2H), 3.50 (dd, J = 13.4, 6.6 Hz, 1H), 3.22 (dd, J = 13.4, 6.8 Hz, 1H), 2.89 (t, J = 11.9 Hz, 2H), 2.43 (s, 4H), 2.20 (d, J = 6.3 Hz, 2H), 1.86 (d, J = 5.7 Hz, 8H), 1.48 (s, 6H), 1.23-1.14 (m, 6H), 1.09-1.05 (m, 3H), 0.79 (d, J = 6.4 Hz, 2H). | 7-1 and 19-4 |
| 121 | 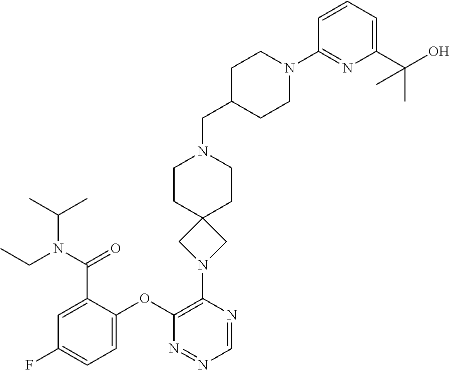 [SMILES: CCN(C(=O)c1cc(F)ccc1Oc1nncnc1N1CC2(CCN(CC3CCN(c4cccc(C(C)(C)O)n4)CC3)CC2)C1)C(C)C] | 661.4 | ¹H NMR (400 MHz, DMSO-d6) δ 8.43 (d, J = 0.6 Hz, 1H), 7.45-7.40 (m, 2H), 7.33 (dd, J = 8.0, 2.4 Hz, 2H), 6.79 (d, J = 7.4 Hz, 1H), 6.59 (d, J = 8.5 Hz, 1H), 4.97 (s, 1H), 4.25-4.18 (m, 4H), 3.78 (s, 2H), 3.63-3.55 (m, 1H), 3.06-3.01 (m, 1H), 2.70 (d, J = 11.7 Hz, 2H), 2.30 (s, 4H), 2.07 (d, J = 6.6 Hz, 2H), 1.72 (s, 8H), 1.34 (s, 6H), 1.11-1.02 (m, 6H), 1.01-0.91 (m, 3H), 0.69 (d, J = 6.1 Hz, 2H). | 7-1 and 27-2 |
| 122 | 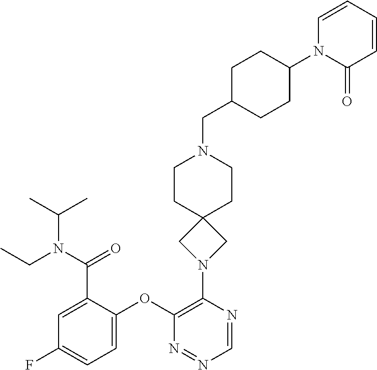 [SMILES: CCN(C(=O)c1cc(F)ccc1Oc1nncnc1N1CC2(CCN(CC3CCC(n4ccccc4=O)CC3)CC2)C1)C(C)C] | 618.4 | ¹H NMR (400 MHz, CD₃OD) δ 8.38 (s, 1H), 7.78-7.68 (m, 1H), 7.51-7.45 (m, 1H), 7.43-7.37 (m, 1H), 7.31-7.25 (m, 1H), 7.24-7.17 (m, 1H), 6.56-6.48 (m, 1H), 6.44-6.39 (m, 1H), 4.85-4.74 (m, 1H), 4.45-4.30 (m, 2H), 4.00-3.89 (m, 2H), 3.85-3.77 (m, 1H), 3.55-3.45 (m, 1H), 3.26-3.18 (m, 1H), 2.58-2.43 (m, 3H), 2.25 (d, J = 6.9 Hz, 1H), 2.06-1.97 (m, 2H), 1.94-1.85 (m, 6H), 1.80-1.65 (m, 4H), 1.29-1.25 (m, 1H), 1.26-1.17 (m, 3H), 1.16-1.13 (m, 5H), 1.06 (t, J = 7.1 Hz, 1H), 0.81 (d, J = 6.0 Hz, 2H). | 7-1 and 37 |
| 123 | 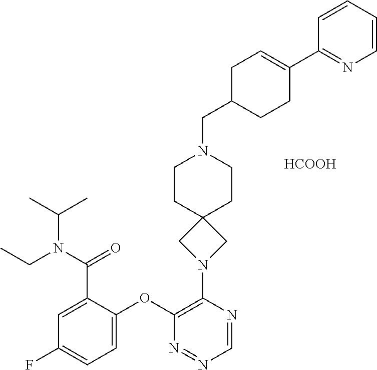 [SMILES: CCN(C(=O)c1cc(F)ccc1Oc1nncnc1N1CC2(CCN(CC3CC=C(c4ccccn4)CC3)CC2)C1)C(C)C ; "HCOOH"] | 600.4 | ¹H NMR (400 MHz, CD₃OD) δ 8.47- 8.45 (m, 1H), 8.42 (s, 1H), 8.34 (s, 1H), 7.82-7.73 (m, 1H), 7.54-7.50 (m, 1H), 7.43-7.38 (m, 1H), 7.33-7.20 (m, 3H), 6.56 (s, 1H), 4.50-4.45 (m, 2H), 4.04 (s, 2H), 3.87-3.77 (m, 1H), 3.55-3.45 (m, 1H), 3.28-3.12 (br, 4H), 3.01 (d, J = 6.6 Hz, 2H), 2.71-2.45 (m, 3H), 2.24-2.18 (m, 1H), 2.17-2.11 (m, 4H), 2.09-2.00 (m, 2H), 1.58-1.48 (m, 1H), 1.23-1.18 (m, 2H), 1.17-1.11 (m, 5H), 1.08 (t, J = 7.1 Hz, 1H), 0.80 (d, J = 5.6 Hz, 2H). | 7-1 and 29 |
| 124 | 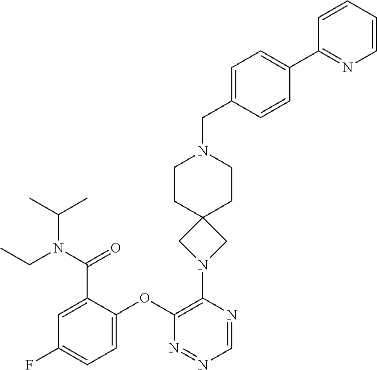 [SMILES: CCN(C(=O)c1cc(F)ccc1Oc1nncnc1N1CC2(CCN(Cc3ccc(-c4ccccn4)cc3)CC2)C1)C(C)C] | 596.4 | ¹H NMR (400 MHz, DMSO-d6) δ 8.63 (d, J = 4.7 Hz, 1H), 8.44-8.42 (m, 1H), 8.02 (d, J = 8.2 Hz, 2H), 7.92 (d, J = 8.1 Hz, 1H), 7.87-7.82 (m, 1H), 7.43-7.37 (m, 3H), 7.33 (dd, J = 11.1, 4.7 Hz, 3H), 4.21 (d, J = 13.8 Hz, 2H), 3.80 (d, J = 7.5 Hz, 2H), 3.62-3.54 (m, 1H), 3.48 (s, 2H), 2.37 (s, 4H), 1.74 (s, 4H), 1.11- 0.98 (m, 6H), 1.00-0.85 (m, 3H), 0.67 (d, J = 6.1 Hz, 2H). | 7-1 and 28 |
| 125 | 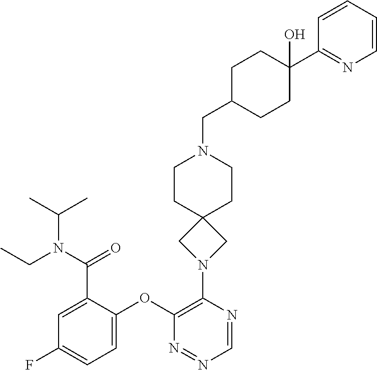 [SMILES: CCN(C(=O)c1cc(F)ccc1Oc1nncnc1N1CC2(CCN(CC3CCC(O)(c4ccccn4)CC3)CC2)C1)C(C)C] | 618.2 | ¹H NMR (400 MHz, CD₃OD) δ 8.38 (s, 1H), 7.43-7.40 (m, 1H), 7.33-7.23 (m, 1H), 7.21-7.18 (m, 1H), 6.68 (s, 1H), 4.39-4.36 (m, 2H), 4.17-4.06 (m, 2H), 3.95 (s, 3H), 3.94-3.89 (m, 2H), 3.88 (s, 3H), 3.83-3.80 (m, 1H), 3.26-3.14 (m, 1H), 2.88-2.85 (m, 2H), 2.46-2.41 (m, 3H), 2.24-2.20 (m, 2H), 1.88-1.83 (m, 7H), 1.37-1.00 (m, 11H), 0.83- 0.79 (m, 2H). | 7-1 and 35 |
| 126 | 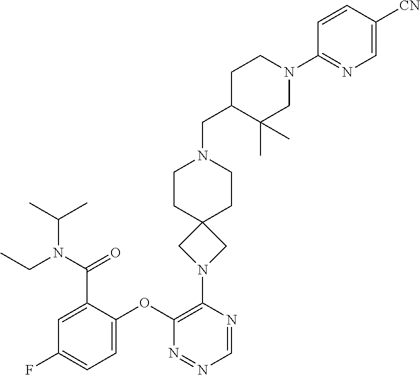 [SMILES: CCN(C(=O)c1cc(F)ccc1Oc1nncnc1N1CC2(CCN(CC3CCN(c4ccc(C#N)cn4)CC3(C)C)CC2)C1)C(C)C] | 656.4 | ¹H NMR (400 MHz, DMSO-d6) δ 8.43 (d, J = 0.7 Hz, 1H), 8.38 (t, J = 3.4 Hz, 1H), 7.73 (dd, J = 9.2, 2.3 Hz, 1H), 7.45- 7.40 (m, 1H), 7.33 (dd, J = 9.9, 5.9 Hz, 2H), 6.91 (d, J = 9.2 Hz, 1H), 4.46 (d, J = 11.5 Hz, 1H), 4.19 (s, 2H), 4.06 (d, J = 12.3 Hz, 1H), 3.78 (s, 2H), 3.64-3.55 (m, 1H), 3.06-3.01 (m, 1H), 2.83 (t, J = 11.5 Hz, 1H), 2.32-2.67 (m, 1H), 2.45- 2.24 (m, 2H), 2.20 (d, J = 12.4 Hz, 1H), 2.06-1.88 (m, 2H), 1.75 (d, J = 24.5 Hz, 6H), 1.55 (s, 1H), 1.10-0.90 (m, 12H), 0.69 (d, J = 6.1 Hz, 2H), 0.66 (s, 3H). | 7-1 and 12-4 |
| 127 | 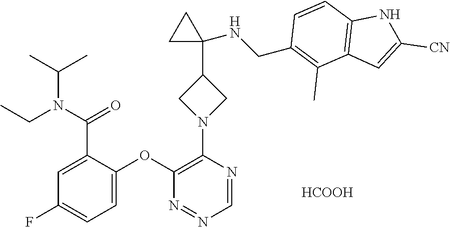 [SMILES: CCN(C(=O)c1cc(F)ccc1Oc1nncnc1N1CC(C2(NCc3ccc4[nH]c(C#N)cc4c3C)CC2)C1)C(C)C ; "HCOOH"] | 583.3 | ¹H NMR (400 MHz, DMSO-d6) δ 12.18 (s, 1H), 8.41 (d, J = 1.3 Hz, 1H), 7.39 (d, J = 4.9 Hz, 2H), 7.34-7.29 (m, 2H), 7.24 (d, J = 8.2 Hz, 1H), 7.16 (d, J = 8.5 Hz, 1H), 4.53 (s, 1H), 4.09 (s, 2H), 3.72 (s, 4H), 3.58 (s, 1H), 3.09 (s, 2H), 2.95 (s, 1H), 2.42 (s, 3H), 1.09-0.98 (m, 6H), 0.96-0.91 (m, 3H), 0.68 (d, J = 5.7 Hz, 2H), 0.63 (s, 2H). | 7-18 |
| 128 | 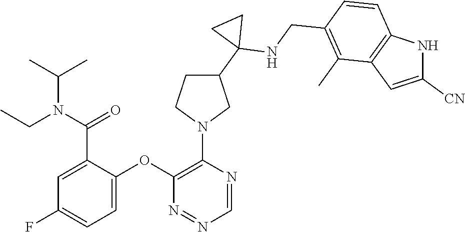 [SMILES: CCN(C(=O)c1cc(F)ccc1Oc1nncnc1N1CCC(C2(NCc3ccc4[nH]c(C#N)cc4c3C)CC2)C1)C(C)C] | 597.6 | 'H NMR (400 MHz, CD₃OD) δ 8.42 (s, 1H), 7.42-7.14 (m, 6H), 4.18-4.15 (m, 1H), 3.96-3.90 (m, 4H), 3.65-3.39 (m, 2H), 3.23-3.20 (m, 1H), 3.01-2.98 (m, 1H), 2.56-2.53 (m, 3H), 2.07-2.03 (m, 1H), 1.67-1.61 (m, 1H), 1.16-1.09 (m, 8H), 0.79-0.74 (m, 6H). | 7-19 |
| 129 | 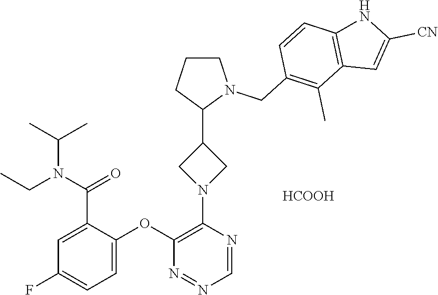 [SMILES: CCN(C(=O)c1cc(F)ccc1Oc1nncnc1N1CC(C2CCCN2Cc2ccc3[nH]c(C#N)cc3c2C)C1)C(C)C ; "HCOOH"] | 597.6 | ¹H NMR (400 MHz, a mixture of CDCl₃ and CD₃OD) δ 8.34 (s, 1H), 8.25 (s, 1H), 7.28-7.19 (m, 5H), 7.05-7.02 (m, 1H), 4.56-4.03 (m, 4H), 3.93-3.86 (m, 3H), 3.43-3.40 (m, 1H), 3.18-3.03 (m, 4H), 2.68-2.65 (m, 1H), 2.54 (s, 3H), 2.19- 2.14 (m, 1H), 1.88-1.83 (m, 3H), 1.11- 1.05 (m, 7H), 0.81-0.75 (m, 2H). | 7-20 |
| 130 | 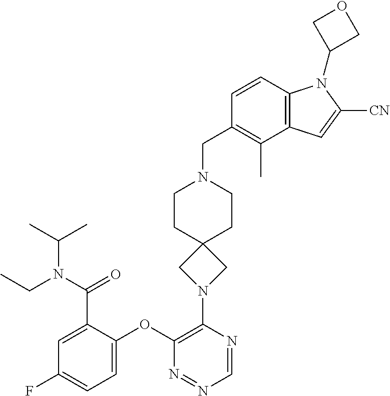 [SMILES: CCN(C(=O)c1cc(F)ccc1Oc1nncnc1N1CC2(CCN(Cc3ccc4c(cc(C#N)n4C4COC4)c3C)CC2)C1)C(C)C] | 653.6 | ¹H NMR (400 MHz, CD₃OD) δ 8.40 (s, 1H), 7.90-7.87 (m, 1H), 7.57-7.46 (m, 2H), 7.42-7.38 (m, 1H), 7.31-7.18 (m, 2H), 5.91-5.80 (m, 1H), 5.25-5.20 (m, 4H), 4.48-4.44 (m, 2H), 4.35-430 (m, 2H), 4.03-4.00 (m, 2H), 3.87-3.75 (m, 1H), 3.47-3.43 (m, 1H), 3.23-3.18 (m, 5H), 2.64 (s, 3H), 2.12-2.07 (m, 4H), 1.15-1.07 (m, 7H), 0.82-0.77 (m, 2H). | 7-1 and 12-7 |
| 174 | 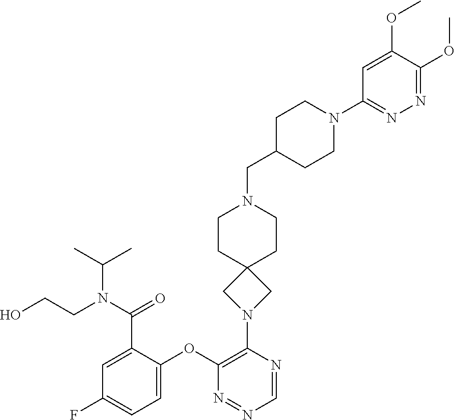 [SMILES: COc1cc(N2CCC(CN3CCC4(CC3)CN(c3ncnnc3Oc3ccc(F)cc3C(=O)N(CCO)C(C)C)C4)CC2)nnc1OC] | 680.5 | ¹H NMR (400 MHz, CD₃OD) δ 8.37 (s, 1H), 7.44-7.34 (m, 1H), 7.26-7.23 (m, 2H), 6.68 (s, 1H), 4.36-4.33 (m, 2H), 4.15-4.11 (m, 2H), 3.95 (s, 3H), 3.94 3.85 (m, 2H), 3.87 (s, 3H), 3.85-3.77 (m, 1H), 3.74-3.70 (m, 1H), 3.55-3.51 (m, 2H), 2.87-2.83 (m, 2H), 2.48-2.38 (m, 4H), 2.29-2.23 (m, 2H), 1.90-1.82 (m, 7H), 1.27-1.21 (m, 5H), 1.16-1.12 (m, 2H), 0.82-0.78 (m, 2H). | 7-25 and 24-3 |

### Compound 131: 2-((5-(7-((2-cyano-3-(3,6-dihydro-2H-pyran-4-yl)-4-methyl-1H-indol-5-yl)methyl)-2,7-diazaspiro[3.5]nonan-2-yl)-1,2,4-triazin-6-yl)oxy)-N-ethyl-5-fluoro-N-isopropylbenzamide

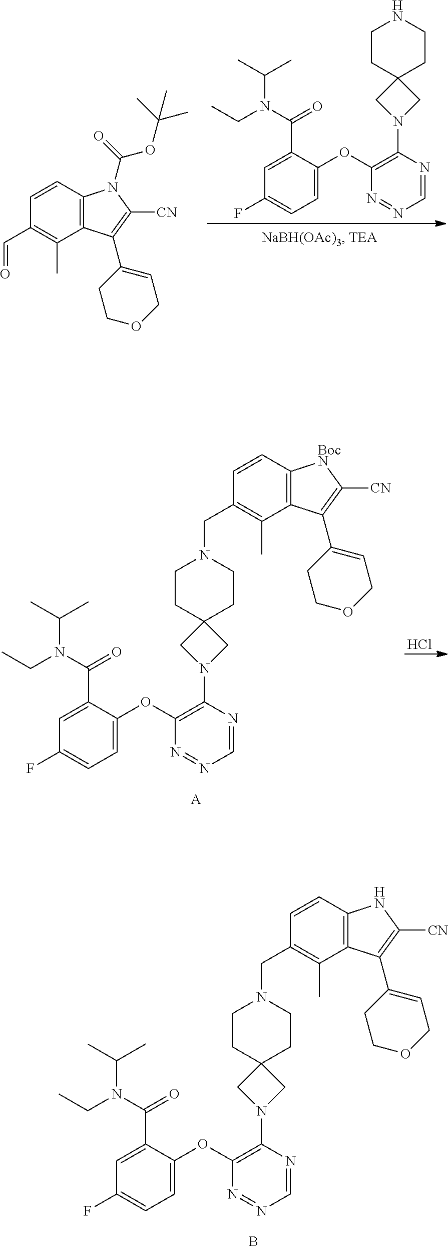

### (A) tert-butyl 2-cyano-3-(3,6-dihydro-2H-pyran-4-yl)-5-((2-(6-(2-(ethyl (isopropyl)carbamoyl)-4-fluorophenoxy)-1,2,4-triazin-5-yl)-2,7-diazaspiro[3.5]nonan-7-yl)methyl)-4-methyl-1H-indole-1-carboxylate

To a reaction flask were added intermediate 16 (86 mg, 0.24 mmol), intermediate 7-1 (50 mg, 0.12 mmol), triethylamine (0.065 ml, 0.47 mmol) and dichloromethane (15 ml), and the mixture was stirred at room temperature for 1 hour. Sodium triacetoxyborohydride (99 mg, 0.47 mmol) was then added, and the mixture was stirred for additional 15 hours. After the reaction was completed, the reaction solution was concentrated, and the residue was purified with flash column chromatography (eluted with a gradient of water/methanol=100:0-0:100) to obtain 38 mg of yellow solid. MS (m/z): 779.4 [M+H]⁺.

### (B) 2-((5-(7-((2-cyano-3-(3,6-dihydro-2H-pyran-4-yl)-4-methyl-1H-indol-5-yl)methyl)-2,7-diazaspiro[3.5]nonan-2-yl)-1,2,4-triazin-6-yl)oxy)-N-ethyl-5-fluoro-N-isopropylbenzamide

To a reaction flask were added tert-butyl 2-cyano-3-(3,6-dihydro-2H-pyran-4-yl)-5-((2-(6-(2-(ethyl(isopropyl)carbamoyl)-4-fluorophenoxy)-1,2,4-triazin-5-yl)-2,7-diazaspiro[3.5]nonan-7-yl)methyl)-4-methyl-1H-indole-1-carboxylate (60 mg, 0.077 mmol) and hydrochloric acid in methanol (5 ml, 6 mole/liter), and the mixture was reacted at room temperature for 1 hour. After the reaction was completed, the reaction solution was concentrated, and the residue was purified with flash column chromatography (eluted with a gradient of dichloromethane/methanol=100:0-0:100) to obtain 40 mg of white solid. MS (m/z): 679.3 [M+H]⁺.

¹H NMR (400 MHz, CD₃OD) δ 12.20 (s, 1H), 8.43 (s, 1H), 8.14 (s, 1H), 7.45-7.38 (m, 1H), 7.37-7.29 (m, 2H), 7.19 (q, J=8.6 Hz, 2H), 5.86 (s, 1H), 4.26-4.15 (m, 4H), 3.87-3.76 (m, 4H), 3.61-3.56 (m, 1H), 3.45 (s, 2H), 3.36-3.32 (m, 1H), 3.10-2.93 (m, 1H), 2.48 (s, 3H), 2.42-2.17 (m, 6H), 1.75-1.63 (br, 4H), 1.11-0.88 (m, 7H), 0.68 (d, J=6.1 Hz, 2H).

### Compound 132: N-ethyl-5-fluoro-2-((5-(7-((1-(6-(hydroxymethyl)pyridin-2-yl)piperidin-4-yl)methyl)-2,7-diazaspiro[3.5]nonan-2-yl)-1,2,4-triazin-6-yl)oxy)-N-isopropylbenzamide

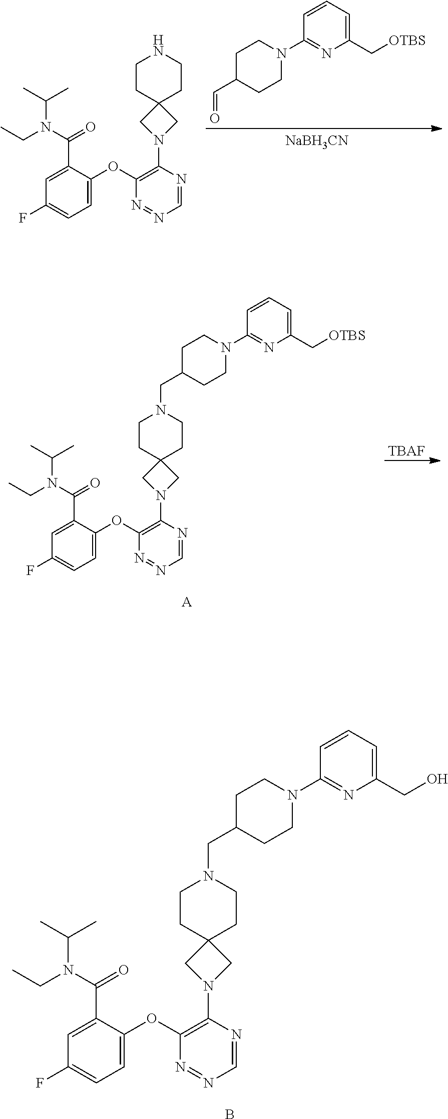

### (A) 2-((5-(7-((1-(6-(((tert-butyldimethylsilyl)oxy)methyl)pyridin-2-yl)piperidin-4-yl)methyl)-2,7-diazaspiro[3.5]nonan-2-yl)-1,2,4-triazin-6-yl)oxy)-N-ethyl-5-fluoro-N-isopropylbenzamide

To a reaction flask were added successively intermediate 7-1 (40 mg, 0.093 mmol), intermediate 27-1 (62 mg, 0.185 mmol), sodium cyanoborohydride (12 mg, 0.191 mmol), methanol (5 ml) and acetic acid (0.5 ml), and the mixture was reacted at room temperature for 5 hours. After the reaction was completed, the reaction solution was concentrated, and the residue was purified with flash column chromatography (eluted with a gradient of water/methanol=100:0-0:100) to obtain 40 mg of white solid. MS (m/z): 747.5 [M+H]⁺.

### (B) N-ethyl-5-fluoro-2-((5-(7-((1-(6-(hydroxymethyl)pyridin-2-yl)piperidin-4-yl)methyl)-2,7-diazaspiro[3.5]nonan-2-yl)-1,2,4-triazin-6-yl)oxy)-N-isopropylbenzamide

To a reaction flask were added successively 2-((5-(7-((1-(6-(((tert-butyldimethylsilyl)oxy)methyl)pyridin-2-yl)piperidin-4-yl)methyl)-2,7-diazaspiro[3.5]nonan-2-yl)-1,2,4-triazin-6-yl)oxy)-N-ethyl-5-fluoro-N-isopropylbenzamide (40 mg, 0.05 mmol), tetrabutylammonium fluoride (30 mg, 0.12 mmol) and tetrahydrofuran (5 ml), and the mixture was reacted at room temperature for 5 hours. After the reaction was completed, the reaction solution was concentrated, and the residue was purified with flash column chromatography (eluted with a gradient of water/methanol=100:0-0:100) to obtain 7 mg of white solid. MS (m/z): 633.3 [M+H]⁺.

¹H NMR (400 MHz, DMSO-d6) δ 8.43 (s, 1H), 7.44 (dd, J=14.1, 6.5 Hz, 2H), 7.35-7.31 (m, 2H), 6.63 (dd, J=15.5, 7.7 Hz, 2H), 5.13 (t, J=5.9 Hz, 1H), 4.34 (d, J=5.9 Hz, 2H), 4.25-4.18 (m, 4H), 3.78 (s, 2H), 3.63-3.55 (m, 1H), 3.03 (d, J=41.9 Hz, 1H), 2.67 (d, J=11.5 Hz, 2H), 2.31 (s, 4H), 2.07 (d, J=6.7 Hz, 2H), 1.71 (d, J=10.3 Hz, 8H), 1.10-1.02 (m, 6H), 1.00-0.91 (m, 3H), 0.70 (d, J=6.5 Hz, 2H).

### Compound 133: N-ethyl-5-fluoro-N-isopropyl-2-((5-(7-((1-(6-vinylpyridazin-3-yl)piperidin-4-yl)methyl)-2,7-diazaspiro[3.5]nonan-2-yl)-1,2,4-triazin-6-yl)oxy)benzamide

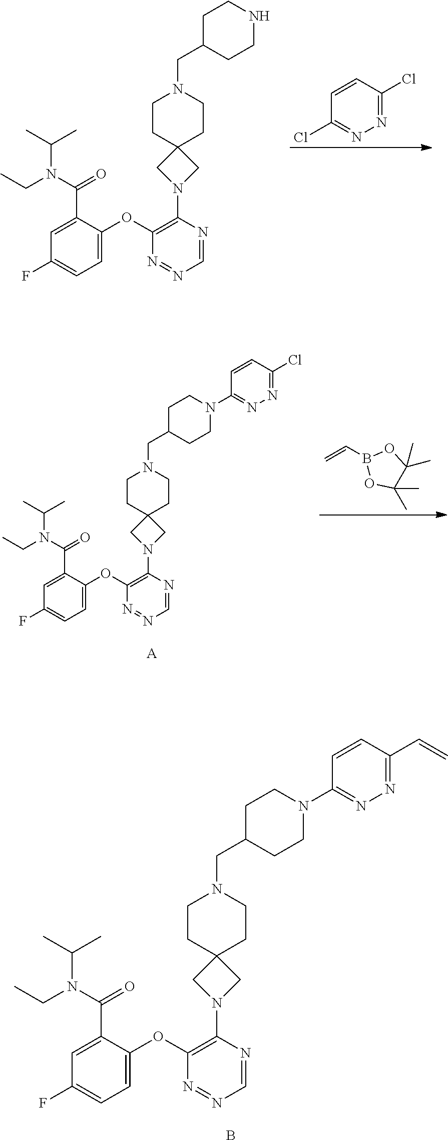

### (A) 2-((5-(7-((1-(6-chloropyridazin-3-yl)piperidin-4-yl)methyl)-2,7-diazaspiro[3.5]nonan-2-yl)-1,2,4-triazin-6-yl)oxy)-N-ethyl-5-fluoro-N-isopropylbenzamide

To a reaction flask were added intermediate 10-1 (350 mg, 0.67 mmol), 3,6-dichloropyridazine (99 mg, 0.67 mmol), potassium carbonate (184 mg, 1.33 mmol) and N,N-dimethylformamide (10 ml), and the mixture was heated to 120° C., stirred and reacted for 2 hours. After the reaction was completed, the reaction solution was purified with flash column chromatography (eluted with a gradient of water/methanol=100:0-0; 100) to obtain 260 mg of yellow solid. MS (m/z): 638.3, 640.3 [M+H]⁺.

### (B) N-ethyl-5-fluoro-N-isopropyl-2-((5-(7-((1-(6-vinylpyridazin-3-yl)piperidin-4-yl)methyl)-2,7-diazaspiro[3.5]nonan-2-yl)-1,2,4-triazin-6-yl)oxy)benzamide

To a reaction flask were added 2-((5-(7-((1-(6-chloropyridazin-3-yl)piperidin-4-yl)methyl)-2,7-diazaspiro[3.5]nonan-2-yl)-1,2,4-triazin-6-yl)oxy)-N-ethyl-5-fluoro-N-isopropylbenzamide (100 mg, 0.16 mmol), 4,4,5,5-tetramethyl-2-vinyl-1,3,2-dioxaborolane (37 mg, 0.24 mmol), potassium carbonate (54 mg, 0.39 mmol), Pd(dppf)Cl₂·CH₂Cl₂ (13 mg, 0.018 mmol), dioxane (20 ml) and water (3 ml), and then the mixture was heated to 100° C., stirred and reacted for 15 hours. After the reaction was completed, the reaction solution was concentrated, and the residue was purified with flash column chromatography (eluted with a gradient of water/methanol=100:0-0:100) to obtain 15 mg of white solid. MS (m/z): 630.6 [M+H]⁺.

¹H NMR (400 MHz, CD₃OD) δ 8.38 (s, 1H), 7.65 (d, J=9.7 Hz, 1H), 7.43-7.40 (m, 1H), 7.28-7.19 (m, 3H), 6.89-6.82 (m, 1H), 5.95 (d, J=17.9 Hz, 1H), 5.45 (d, J=11.2 Hz, 1H), 4.43-4.32 (m, 4H), 3.97-3.86 (m, 2H), 3.84-3.78 (m, 1H), 3.58-3.45 (m, 1H), 3.28-3.17 (m, 1H), 2.97 (t, J=12.0 Hz, 2H), 2.60-2.30 (m, 4H), 2.22 (d, J=6.4 Hz, 2H), 1.92-1.85 (m, 7H), 1.25-1.19 (m, 3H), 1.18-1.10 (m, 5H), 1.06 (t, J=7.1 Hz, 1H), 0.81 (d, J=5.9 Hz, 2H).

The following compounds were prepared according to the preparation procedure of compound 133 using corresponding intermediates and reagents under appropriate conditions that will be recognized by those skilled in the art.

| | | MS | | Intermediate |
|---|---|---|---|---|
| Compound | Structural formula | (M + H)+ | ¹HNMR | used |
| 134 | 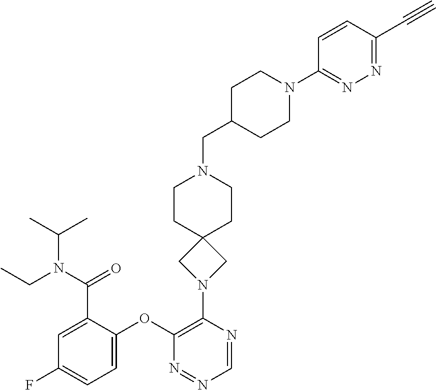 [SMILES: C#Cc1ccc(N2CCC(CN3CCC4(CC3)CN(c3ncnnc3Oc3ccc(F)cc3C(=O)N(CC)C(C)C)C4)CC2)nn1] | 628.6 | ¹H NMR (400 MHz, CD₃OD) δ 8.38 (s, 1H), 7.45-7.38 (m, 2H), 7.30-7.23 (m, 1H), 7.22-7.18 (m, 2H), 4.45 (d, J = 13.2 Hz, 2H), 4.40-4.31 (m, 2H), 3.98- 3.87 (m, 2H), 3.85-3.74 (m, 1H), 3.57- 3.44 (m, 1H), 3.27-3.16 (m, 1H), 2.99 (t, J = 11.9 Hz, 2H), 2.45 (s, 4H), 2.21 (d, J = 6.6 Hz, 2H), 1.96-1.84 (m, 7H), 1.26-1.12 (m, 8H), 1.06 (t, J = 7.1 Hz, 1H), 0.81 (d, J = 6.0 Hz, 2H). | 10-1 |

### Compound 135: N-ethyl-5-fluoro-N-isopropyl-2-((5-(7-((4-(pyridin-2-yl)cyclohexyl)methyl)-2,7-diazaspiro[3.5]nonan-2-yl)-1,2,4-triazin-6-yl)oxy)benzamide

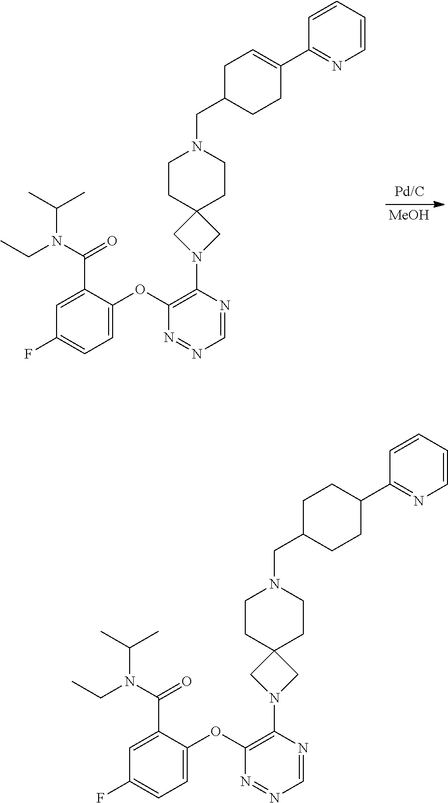 [SMILES: "MeOH" ; CCN(C(=O)c1cc(F)ccc1Oc1nncnc1N1CC2(CCN(CC3CC=C(c4ccccn4)CC3)CC2)C1)C(C)C ; CCN(C(=O)c1cc(F)ccc1Oc1nncnc1N1CC2(CCN(CC3CCC(c4ccccn4)CC3)CC2)C1)C(C)C]

To a reaction flask were added compound 123 (100 mg, 0.167 mmol), palladium carbon (15 mg) and methanol (15 ml), and the mixture was reacted under hydrogen balloon at room temperature for 15 hours. After the reaction was completed, the reaction solution was filtered, the filtrate was concentrated, and the residue was purified with flash column chromatography (eluted with a gradient of water/methanol=100:0-0:100) to obtain 80 mg of white solid. MS (m/z): 602.4 [M+H]⁺.

¹H NMR (400 MHz, CD₃OD) δ 8.48-8.43 (m, 1H), 8.43 (s, 1H), 8.35 (s, 1H), 7.82-7.72 (m, 1H), 7.44-7.20 (m, 5H), 4.47 (s, 2H), 4.20-4.05 (m, 2H), 3.88-3.75 (m, 1H), 3.55-3.42 (m, 1H), 3.30-3.17 (m, 4H), 2.98 (d, J=5.4 Hz, 1H), 2.85-2.68 (m, 1H), 2.32-2.24 (m, 1H), 2.21-2.15 (m, 4H), 2.04-1.95 (m, 2H), 1.88-1.73 (m, 5H), 1.72-1.61 (m, 1H), 1.32-1.26 (m, 1H), 1.25-1.11 (m, 7H), 1.08 (t, J=7.1 Hz, 1H), 0.80 (d, J=5.7 Hz, 2H).

### Compound 136: 6-(4-((2-(6-((5,6′-difluoro-5′-hydroxy-2′,3′,4′,5′-tetrahydro-[1,1′-biphenyl]-2-yl)oxy)-1,2,4-triazin-5-yl)-2,7-diazaspiro[3.5]nonan-7-yl)methyl)piperidin-1-yl)nicotinonitrile

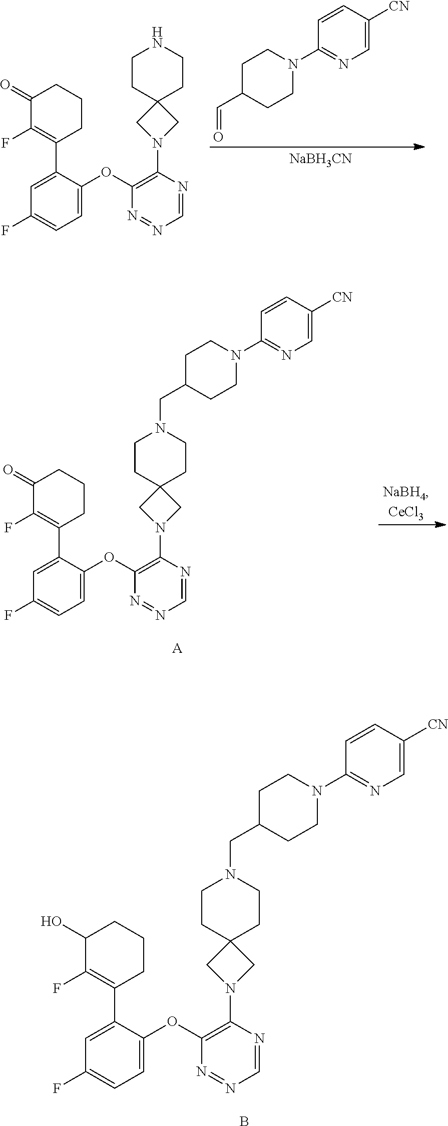

### (A) 6-(4-((2-(6-((5,6′-difluoro-5′-oxo-2′,3′,4′,5′-tetrahydro-[1,1′-biphenyl]-2-yl)oxy)-1,2,4-triazin-5-yl)-2,7-diazaspiro[3.5]nonan-7-yl)methyl)piperidin-1-yl)nicotinonitrile

To a reaction flask were added successively intermediate 7-15 (80 mg, 0.19 mmol), intermediate 12-6 (65 mg, 0.30 mmol), methanol (5 ml) and sodium cyanoborohydride (25 mg, 0.40 mmol), and the mixture was reacted at room temperature for 2 hours. The reaction solution was quenched with a saturated aqueous ammonium chloride solution (2 ml), extracted three times with dichloromethane, dried over anhydrous sodium sulfate, and concentrated to obtain a residue, which was purified with flash column chromatography (eluted with a gradient of water/methanol=100:0-0:100) to obtain 49 mg of yellow solid. MS (m/z): 627.8 [M+H]⁺.

### (B) 6-(4-((2-(6-((5,6′-difluoro-5′-hydroxy-2′,3′,4′,5′-tetrahydro-[1,1′-biphenyl]-2-yl)oxy)-1,2,4-triazin-5-yl)-2,7-diazaspiro[3.5]nonan-7-yl)methyl)piperidin-1-yl)nicotinonitrile

To a reaction flask were added successively 6-(4-((2-(6-((5,6′-difluoro-5′-oxo-2′,3′,4′,5′-tetrahydro-[1,1′-biphenyl]-2-yl)oxy)-1,2,4-triazin-5-yl)-2,7-diazaspiro[3.5]nonan-7-yl)methyl)piperidin-1-yl)nicotinonitrile (49 mg, 0.08 mmol), methanol (5 ml), cerium trichloride (19 mg, 0.08 mmol) and sodium borohydride (3 mg, 0.08 mmol), and the mixture was reacted under an ice-bath for 1 hour. The reaction solution was quenched with a saturated aqueous ammonium chloride solution (2 ml), extracted three times with dichloromethane, dried over anhydrous sodium sulfate, and concentrated to obtain a residue, which was purified with flash column chromatography (eluted with a gradient of water/methanol=100:0-0:100) to obtain 15.6 mg of yellow solid. MS (m/z): 629.8 [M+H]⁺.

¹H NMR (400 MHz, CD₃OD) δ 8.35 (d, J=2.3 Hz, 1H), 8.33 (s, 1H), 7.68-7.64 (m, 1H), 7.26-7.23 (m, 1H), 7.16-7.01 (m, 2H), 6.82 (d, J=9.2 Hz, 1H), 4.51-4.41 (m, 3H), 4.38-4.35 (m, 1H), 4.19-4.12 (m, 1H), 3.94-3.90 (m, 2H), 2.95 (t, J=11.8 Hz, 2H), 2.45-2.40 (m, 4H), 2.20-2.15 (m, 4H), 1.96-1.82 (m, 8H), 1.73-1.66 (m, 2H), 1.53-1.48 (m, 1H), 1.20-1.08 (m, 2H).

### Compound 137: N-ethyl-5-fluoro-N-isopropyl-2-((5-(7-((1-(oxetan-3-yl)piperidin-4-yl)methyl)-2,7-diazaspiro[3.5]nonan-2-yl)-1,2,4-triazin-6-yl)oxy)benzamide

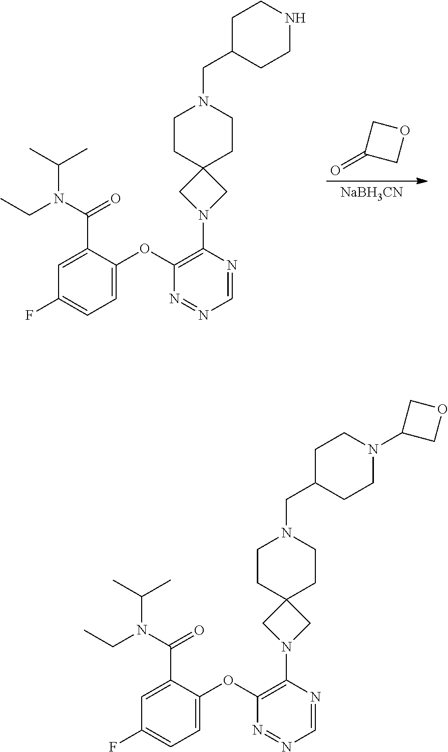

To a reaction flask were added successively intermediate 10-1 (45 mg, 0.086 mmol), oxetane-3-one (12 mg, 0.17 mmol), sodium cyanoborohydride (11 mg, 0.18 mmol), methanol (5 ml) and acetic acid (0.5 ml), and the mixture was reacted at room temperature for 5 hours. After the reaction was completed, the reaction solution was concentrated, and the residue was purified with flash column chromatography (eluted with a gradient of water/methanol=100:0-0:100) to obtain 12.4 mg of oily liquid. MS (m/z): 582.4 [M+H]⁺.

¹H NMR (400 MHz, DMSO-d6) δ 8.43 (d, J=0.9 Hz, 1H), 7.43-7.39 (m, 1H), 7.35-7.31 (m, 2H), 4.48 (t, J=6.2 Hz, 2H), 4.36 (d, J=6.0 Hz, 2H), 4.18 (s, 2H), 3.77 (s, 2H), 3.59 (dt, J=13.1, 6.6 Hz, 1H), 3.06-3.01 (m, 2H), 2.61 (d, J=11.3 Hz, 2H), 2.27 (s, 4H), 2.04 (d, J=7.1 Hz, 2H), 1.67-1.62 (m, 10H), 1.10-1.03 (m, 6H), 1.00-0.93 (m, 3H), 0.69 (d, J=6.1 Hz, 2H).

The following compounds were prepared according to the preparation procedure of compound 137 using corresponding intermediates and reagents under appropriate conditions that will be recognized by those skilled in the art.

| | | MS | | Intermediate |
|---|---|---|---|---|
| Compound | Structural formula | (M + H)⁺ | ¹HNMR | used |
| 138 | 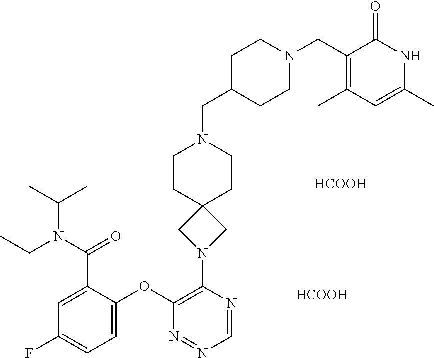 [SMILES: CCN(C(=O)c1cc(F)ccc1Oc1nncnc1N1CC2(CCN(CC3CCN(Cc4c(C)cc(C)[nH]c4=O)CC3)CC2)C1)C(C)C ; "HCOOH" ; "HCOOH"] | 661.4 | ¹H NMR (400 MHz, CD₃OD) δ 8.40 (s, 1H), 8.31 (s, 2H), 7.42-7.38 (m, 1H), 7.29-7.26 (m, 1H), 7.22-7.18 (m, 1H), 6.19 (s, 1H), 4.44-4.40 (m, 2H), 4.19 (s, 2H), 3.99-3.95 (m, 2H), 3.87-3.76 (m, 1H), 3.52-3.48 (m, 2H), 3.25-3.22 (m, 1H), 3.10-3.06 (m, 2H), 2.84-2.78 (m, 4H), 2.62-2.58 (m, 2H), 2.31 (s, 3H), 2.29-2.24 (m, 4H), 2.08-1.93 (m, 7H), 1.60-1.46 (m, 2H), 1.24-1.04 (m, 7H), 0.82-0.77 (m, 2H). | 10-1 |

### Compound 139: 2-((5-(3-(1-(((2-cyano-4-methyl-1H-indol-5-yl)methyl)(methyl) amino)cyclopropyl)azetidin-1-yl)-1,2,4-triazin-6-yl)oxy)-N-ethyl-5-fluoro-N-isopropylbenzamide

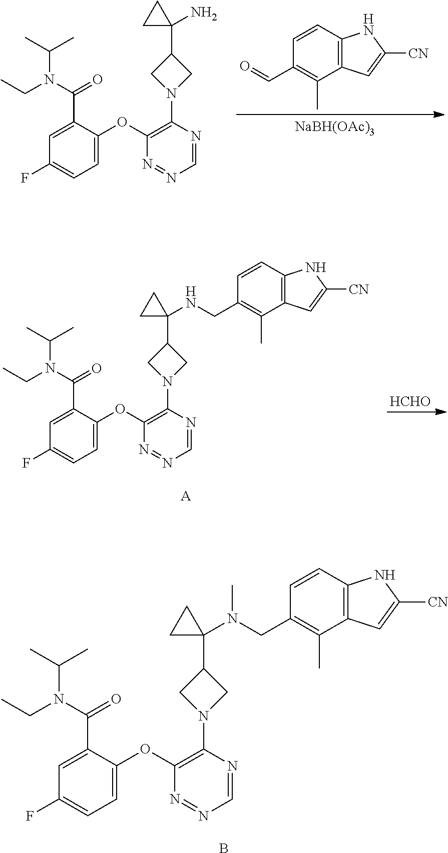

### (A) 2-((5-(3-(1-(((2-cyano-4-methyl-1H-indol-5-yl)methyl)amino)cyclopropyl) azetidin-1-yl)-1,2,4-triazin-6-yl)oxy)-N-ethyl-5-fluoro-N-isopropylbenzamide

To a reaction flask were added successively intermediate 7-18 (55 mg, 0.13 mmol), 5-formyl-4-methyl-1H-indole-2-carbonitrile (25 mg, 0.14 mmol), triethylamine (54 mg, 0.53 mmol), sodium triacetoxyborohydride (112 mg, 0.53 mmol) and dichloromethane (8 ml), and the mixture was reacted at room temperature for 16 hours. After the reaction was completed, the reaction solution was concentrated, and the residue was purified with flash column chromatography (eluted with a gradient of water/methanol=100:0-0:100) to obtain 28 mg of white solid. MS (m/z): 583.3 [M+H]⁺.

### (B) 2-((5-(3-(1-(((2-cyano-4-methyl-1H-indol-5-yl)methyl)(methyl)amino) cyclopropyl)azetidin-1-yl)-1,2,4-triazin-6-yl)oxy)-N-ethyl-5-fluoro-N-isopropylbenzamide

To a reaction flask were added successively 2-((5-(3-(1-(((2-cyano-4-methyl-1H-indol-5-yl)methyl)amino)cyclopropyl)azetidin-1-yl)-1,2,4-triazin-6-yl)oxy)-N-ethyl-5-fluoro-N-isopropylbenzamide (10 mg, 0.017 mmol), an aqueous carbaldehyde solution (0.2 ml), sodium cyanoborohydride (2 mg, 0.032 mmol), methanol (5 ml) and acetic acid (0.5 ml), and the mixture was reacted at room temperature for 16 hours. After the reaction was completed, the reaction solution was concentrated, and the residue was purified with flash column chromatography (eluted with a gradient of water/methanol=100:0-0:100) to obtain 7.4 mg of white solid. MS (m/z): 597.3 [M+H]⁺.

¹H NMR (400 MHz, DMSO-d6) δ 12.23 (s, 1H), 8.43 (s, CH), 7.44-7.39 (m, 2H), 7.34-7.28 (m, 2H), 7.15 (t, J=5.6 Hz, 2H), 4.60 (s, 1H), 4.20 (s, 2H), 3.73-3.53 (m, 6H), 3.13 (s, 1H), 2.42 (s, 3H), 2.01 (s, 3H), 1.09-1.04 (m, 6H), 0.92-0.79 (i, 3H), 0.69 (d, J=5.5 Hz, 2H), 0.65 (s, 2H).

The following compounds were prepared according to the preparation procedure of compound 139 using corresponding intermediates and reagents under appropriate conditions that will be recognized by those skilled in the art.

| | | MS | | Intermediate |
|---|---|---|---|---|
| Compound | Structural formula | (M + H)⁺ | ¹HNMR | used |
| 140 | 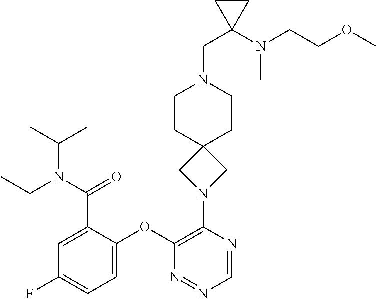 [SMILES: CCN(C(=O)c1cc(F)ccc1Oc1nncnc1N1CC2(CCN(CC3(N(C)CCOC)CC3)CC2)C1)C(C)C] | 570.8 | ¹H NMR (400 MHz, CD₃OD) δ 8.40- 8.31 (m, 1H), 7.44-7.37 (m, 1H), 7.31- 7.15 (m, 2H), 4.41-4.20 (m, 2H), 3.90 (d, J = 7.9 Hz, 2H), 3.83-3.70 (m, 1H), 3.57-3.46 (m, 1H), 3.43 (t, J = 5.9 Hz, 2H), 3.32 (s, 3H), 3.28-3.17 (m, 1H), 3.00 (t, J = 5.9 Hz, 2H), 2.50 (s, 3H), 2.55-2.30(br, 4H) 2.42 (s, 2H), 1.85-1.78 (m, 4H), 1.23-1.12 (m, 6H), 1.06 (t, J = 7.1 Hz, 1H), 0.80 (d, J = 6.3 Hz, 2H), 0.66-0.62 (m, 2H), 0.47- 0.44 (m, 2H). | 10-3 |
| 141 | 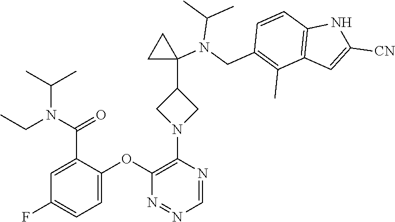 [SMILES: CCN(C(=O)c1cc(F)ccc1Oc1nncnc1N1CC(C2(N(Cc3ccc4[nH]c(C#N)cc4c3C)C(C)C)CC2)C1)C(C)C] | 625.3 | ¹H NMR (400 MHz, CD₃OD) δ 8.39 (s, 1H), 7.38-7.34 (m, 2H), 7.30-7.12 (m, 4H), 4.73-4.69 (m, 2H), 4.26- 4.21 (m, 2H), 4.09-4.06 (m, 2H), 3.79- 3.76 (m, 2H), 3.39-3.35 (m, 2H), 3.23-3.03 (m, 1H), 2.52 (s, 3H), 1.30- 1.04 (m, 13H), 0.79-0.73 (m, 6H). | 7-18 |
| 142 | 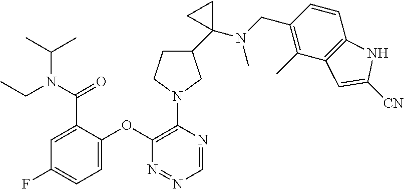 [SMILES: CCN(C(=O)c1cc(F)ccc1Oc1nncnc1N1CCC(C2(N(C)Cc3ccc4[nH]c(C#N)cc4c3C)CC2)C1)C(C)C] | 611.8 | ¹H NMR (400 MHz, CD₃OD) δ 8.41 (s, 1H), 7.36-7.33 (m, 1H), 7.31-7.11 (m, 5H), 4.13-4.08 (m, 1H), 3.90- 3.85 (m, 4H), 3.51-3.47 (m, 2H), 3.19- 3.15 (m, 1H), 3.05-3.02 (m, 1H), 2.55-2.38 (m, 3H), 2.23 (s, 3H), 2.06- 2.01 (m, 1H), 1.46-1.40 (m, 1H), 1.16- 1.11 (m, 7H), 0.89-0.57 (m, 7H). | 7-19 |
| 143 | 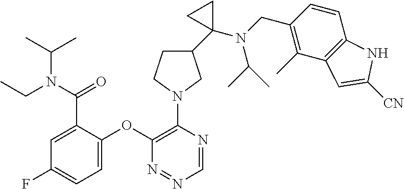 [SMILES: CCN(C(=O)c1cc(F)ccc1Oc1nncnc1N1CCC(C2(N(Cc3ccc4[nH]c(C#N)cc4c3C)C(C)C)CC2)C1)C(C)C] | 639.4 | ¹H NMR (400 MHz, CD₃OD) δ 8.37 (s, 1H), 7.43-7.40 (m, 1H), 7.15-7.09 (m, 5H), 4.46-4.38 (m, 4H), 3.98- 3.78 (m, 4H), 3.46-3.43 (m, 1H), 3.19- 3.15 (m, 2H), 2.78-2.73 (m, 1H), 2.50 (s, 2H), 2.33 (s, 1H), 2.01-1.97 (m, 1H), 1.46-1.41 (m, 1H), 1.12- 1.04 (m, 12H), 0.83-0.72 (m, 4H), 0.58-0.52 (m, 2H). | 7-19 |

### Compound 144: N-ethyl-5-fluoro-N-isopropyl-2-((5-(7-((1-(2-methoxy-N-methylacetamido)cyclopropyl)methyl)-2,7-diazaspiro[3.5]nonan-2-yl)-1,2,4-triazin-6-yl)oxy)benzamide

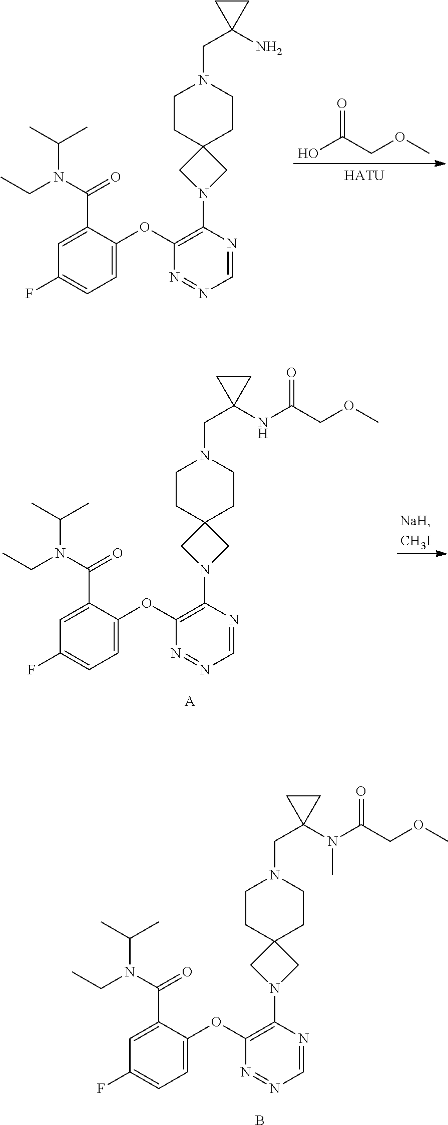

### (A) N-ethyl-5-fluoro-N-isopropyl-2-((5-(7-((1-(2-methoxyacetamido) cyclopropyl)methyl)-2,7-diazaspiro[3.5]nonan-2-yl)-1,2,4-triazin-6-yl)oxy) benzamide

To a reaction flask were added intermediate 10-3 (50 mg, 0.1 mmol), 2-methoxyacetic acid (9 mg, 0.1 mmol), triethylamine (20 mg, 0.2 mmol), HATU (57 mg, 0.15 mmol) and N,N-dimethylformamide (5 ml), and the mixture was stirred and reacted for 15 hours at room temperature. After the reaction was completed, the reaction solution was purified with flash column chromatography (eluted with a gradient of water/methanol=100:0-0:100) to obtain 40 mg of yellow solid. MS (m/z): 570.4 [M+H]⁺.

### (B) N-ethyl-5-fluoro-N-isopropyl-2-((5-(7-((1-(2-methoxy-N-methylacetamido) cyclopropyl)methyl)-2,7-diazaspiro[3.5]nonan-2-yl)-1,2,4-triazin-6-yl)oxy) benzamide

To a reaction flask were added N-ethyl-5-fluoro-N-isopropyl-2-((5-(7-((1-(2-methoxyacetamido)cyclopropyl)methyl)-2,7-diazaspiro[3.5]nonan-2-yl)-1,2,4-triazin-6-yl)oxy)benzamide (40 mg, 0.07 mmol) and N,N-dimethylformamide (5 ml). Sodium hydride (4.2 mg, 0.105 mmol) was added at room temperature, and the mixture was stirred and reacted for half an hour at room temperature. Iodomethane (10 mg, 0.07 mmol) was then added, and the mixture was stirred for additional 15 hours. After the reaction was completed, the reaction solution was purified with flash column chromatography (eluted with a gradient of water/methanol=100:0-0:100) to obtain 25 mg of white solid. MS (m/z): 584.4 [M+H]⁺.

¹H NMR (400 MHz, DMSO-d6) δ 8.46 (s, 1H), 8.40-8.37 (m, 1H), 7.44-7.36 (m, 1H), 7.32-7.17 (m, 2H), 4.51-4.16 (m, 3H), 4.08 (s, 1H), 4.01-3.76 (m, 3H), 3.52-3.45 (m, 1H), 3.42-3.38 (m, 3H), 3.25-3.03 (m, 4H), 3.01-2.96 (m, 3H), 2.95-2.91 (m, 1H), 2.60-2.40 (m, 2H), 2.15-1.97 (m, 3H), 1.88-1.81 (m, 1H), 1.23-1.18 (m, 2H), 1.17-1.11 (m, 4H), 1.10-1.01 (m, 2H), 0.99-0.86 (m, 2H), 0.85-0.70 (m, 3H).

### Compound 145: 3-(((1-(1-(6-(2-(ethyl(isopropyl)carbamoyl)-4-fluorophenoxy)-1,2,4-triazin-5-yl)azetidin-3-yl)cyclopropyl)(methyl)amino)methyl)-1-methyl-1H-pyrazole-5-carboxamide

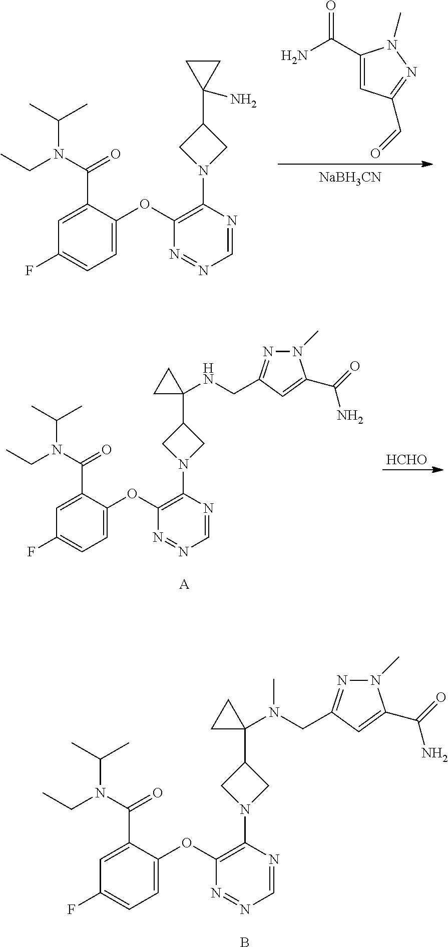

### (A) 3-(((1-(1-(6-(2-(ethyl(isopropyl)carbamoyl)-4-fluorophenoxy)-1,2,4-triazin-5-yl)azetidin-3-yl)cyclopropyl)amino)methyl)-1-methyl-1H-pyrazole-5-carboxamide

To a reaction flask were added successively intermediate 7-18 (150 mg, 0.36 mmol), intermediate 36 (110 mg, 0.72 mmol), triethylamine (1 ml), sodium cyanoborohydride (113 mg, 1.8 mmol) and methanol (10 ml), and the mixture was reacted at room temperature for 2 hours. After the reaction was completed, the reaction solution was concentrated, and the residue was purified with flash column chromatography (eluted with a gradient of water/methanol=100:0-0:100) to obtain 90 mg of solid. MS (m/z): 552.2 [M+H]⁺.

### (B) 3-(((1-(1-(6-(2-(ethyl(isopropyl)carbamoyl)-4-fluorophenoxy)-1,2,4-triazin-5-yl)azetidin-3-yl)cyclopropyl)(methyl)amino)methyl)-1-methyl-1H-pyrazole-5-carboxamide

To a reaction flask were added successively 3-(((1-(1-(6-(2-(ethyl(isopropyl) carbamoyl)-4-fluorophenoxy)-1,2,4-triazin-5-yl)azetidin-3-yl)cyclopropyl)amino) methyl)-1-methyl-1H-pyrazole-5-carboxamide (80 mg, 0.15 mmol), an aqueous carbaldehyde solution (1 ml), sodium cyanoborohydride (45 mg, 0.72 mmol) and methanol (5 ml), and the mixture was reacted at room temperature for 1 hour. After the reaction was completed, the reaction solution was concentrated, and the residue was purified with flash column chromatography (eluted with a gradient of water/methanol=100:0-0:100) to obtain 63 mg of white solid. MS (m/z): 566.8 [M+H]⁺.

¹H NMR (400 MHz, CD₃OD) δ 8.37 (s, 1H), 7.45-7.35 (m, 1H), 7.31-7.17 (m, 2H), 6.69 (s, 1H), 4.72 (s, 1H), 4.33-4.19 (m, 2H), 4.05 (s, 3H), 3.86-3.74 (m, 2H), 3.72-3.56 (m, 4H), 3.54-3.42 (m, 1H), 2.26 (s, 3H), 1.32-2.26 (m, 2H), 1.23-1.11 (m, 7H), 1.05 (t, J=7.1 Hz, 1H), 0.81-0.76 (m, 5H).

### Compound 146: 2-((5-(3-(1-(((5-cyano-1-methyl-1H-pyrazol-3-yl)methyl)(methyl) amino)cyclopropyl)azetidin-1-yl)-1,2,4-triazin-6-yl)oxy)-N-ethyl-5-fluoro-N-isopropylbenzamide

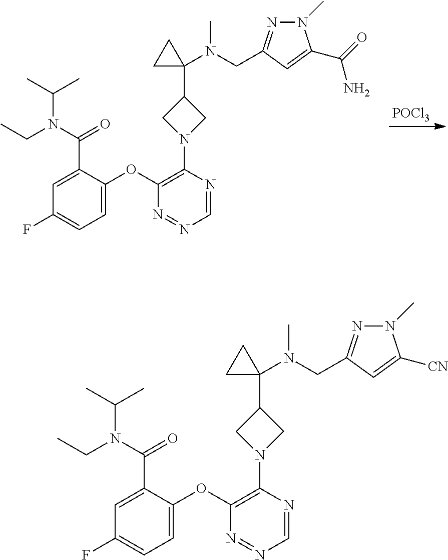

To a reaction flask were added successively compound 145 (50 mg, 0.088 mmol) and pyridine (2 ml), two drops of phosphorus oxychloride were added at 0° C., and the mixture was reacted at room temperature for 1 hour. After the reaction was completed, the reaction solution was concentrated, and the residue was purified with flash column chromatography (eluted with a gradient of water/methanol=100:0-0:100) to obtain 19 mg of white solid. MS (m/z): 548.6 [M+H]⁺.

¹H NMR (400 MHz, CD₃OD) δ 8.39 (s, 1H), 7.43-7.36 (m, 1H), 7.31-7.17 (m, 2H), 6.82 (s, 1H), 4.80-4.65 (m, 1H), 4.35-4.22 (m, 2H), 3.98 (s, 3H), 3.86-3.76 (m, 4H), 3.72-2.64 (m, 1H), 3.52-3.43 (m, 1H), 2.31 (s, 3H), 1.33-1.27 (m, 2H), 1.20 (d, J=6.9 Hz, 2H), 1.17-1.12 ms, 4H), 1.06 (t, J=7.1 Hz, 1H), 0.83-0.77 (m, 5H).

### Compound 147: 2-((5-(7-((1-(3-amino-2-hydroxy-3-oxopropyl)piperidin-4-yl)methyl)-2,7-diazaspiro[3.5]nonan-2-yl)-1,2,4-triazin-6-yl)oxy)-N-ethyl-5-fluoro-N-isopropylbenzamide

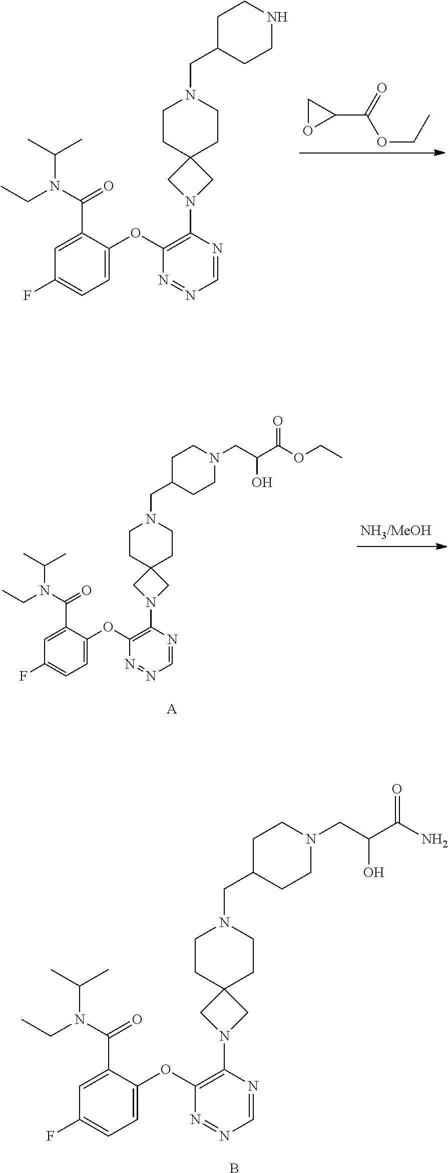

### (A) ethyl 3-(4-((2-(6-(2-(ethyl(isopropyl)carbamoyl)-4-fluorophenoxy)-1,2,4-triazin-5-yl)-2,7-diazaspiro[3.5]nonan-7-yl)methyl)piperidin-1-yl)-2-hydroxypropanoate

To a reaction flask were added successively intermediate 10-1 (100 mg, 0.19 mmol), ethyl oxiran-2-carboxylate (44 mg, 0.38 mmol), triethylamine (38 mg, 0.38 mmol) and methanol (5 ml), and the mixture was heated to 80° C. and reacted for 16 hours. After the reaction was completed, the reaction solution was concentrated, and the residue was purified with flash column chromatography (eluted with a gradient of water/methanol=100:0-0:100) to obtain 40 mg of oily liquid. MS (m/z): 642.4 [M+H]⁺.

### (B) 2-((5-(7-((1-(3-amino-2-hydroxy-3-oxopropyl)piperidin-4-yl)methyl)-2,7-diazaspiro[3.5]nonan-2-yl)-1,2,4-triazin-6-yl)oxy)-N-ethyl-5-fluoro-N-isopropylbenzamide

To a reaction flask were added successively ethyl 3-(4-((2-(6-(2-(ethyl(isopropyl) carbamoyl)-4-fluorophenoxy)-1,2,4-triazin-5-yl)-2,7-diazaspiro[3.5]nonan-7-yl)methyl) piperidin-1-yl)-2-hydroxypropanoate (40 mg, 0.062 mmol) and ammonia in methanol (10 ml), and the mixture was heated to 100° C. and reacted for 16 hours. After the reaction was completed, the reaction solution was concentrated, and the residue was purified with flash column chromatography (eluted with a gradient of water/methanol=100:0-0:100) to obtain 6 mg of white solid. MS (m/z): 613.4 [M+H]⁺.

¹H NMR (400 MHz, CD₃OD) δ 8.36 (s, 1H), 7.39 (dt, J=9.4, 4.8 Hz, 1H), 7.30-7.23 (m, 1H), 7.19 (dd, J=8.0, 3.0 Hz, 1H), 4.35 (d, J=13.6 Hz, 2H), 4.14 (dd, J=8.5, 3.5 Hz, 1H), 3.91 (d, J=8.1 Hz, 2H), 3.80 (dt, J=13.2, 6.6 Hz, 1H), 2.99-2.96 (m, 2H), 2.68 (dd, J=13.1, 3.5 Hz, 1H), 2.55 (dd, J=12.0, 7.5 Hz, 1H), 2.33-2.28 (m, 4H), 2.18 (d, J=6.5 Hz, 2H), 2.13-2.07 (m, 1H), 1.86 (d, J=8.3 Hz, 4H), 1.74 (s, 2H), 1.56 (s, 2H), 1.21 (t, J=11.1 Hz, 4H), 1.12-1.08 (m, 6H), 0.88-0.80 (m, 3H).

The following compounds were prepared according to the preparation procedure of compound 147 using corresponding intermediates and reagents under appropriate conditions that will be recognized by those skilled in the art.

| | | MS | | Intermediate |
|---|---|---|---|---|
| Compound | Structural formula | (M + H)⁺ | ¹HNMR | used |
| 148 | 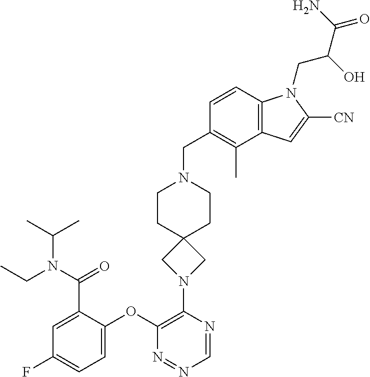 [SMILES: CCN(C(=O)c1cc(F)ccc1Oc1nncnc1N1CC2(CCN(Cc3ccc4c(cc(C#N)n4CC(O)C(N)=O)c3C)CC2)C1)C(C)C] | 684.4 | ¹H NMR (400 MHz, CD₃OD) δ 8.36 (d, J = 1.1 Hz, 1H), 7.40 (dd, J = 9.1, 4.2 Hz, 2H), 7.33-7.30 (m, 2H), 7.29- 7.24 (m, 1H), 7.22-7.17 (m, 1H), 4.64 (dt, J = 9.4, 4.7 Hz, 2H), 4.42-4.32 (m, 4H), 3.91 (d, J = 7.8 Hz, 2H), 3.58 (s, 2H), 2.53 (s, 3H), 2.37 (s, 4H), 2.22- 2.11 (m, 1H), 2.06-1.94 (m, 1H), 1.82 (s, 4H), 1.12-1.05 (m, 6H), 0.90- 0.78 (m, 3H). | 7-1 and 17 |

### Compound 149: 6-(4-((2-(6-(2-(ethyl(isopropyl)carbamoyl)-4-fluorophenoxy)-1,2,4-triazin-5-yl)-2,7-diazaspiro[3.5]nonan-7-yl)methyl)piperidin-1-yl)nicotinamide formate

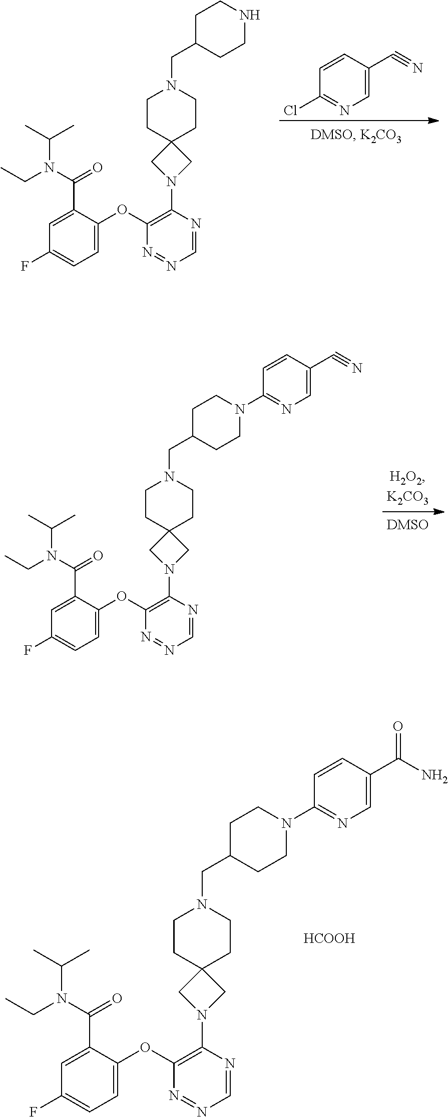 [SMILES: CCN(C(=O)c1cc(F)ccc1Oc1nncnc1N1CC2(CCN(CC3CCNCC3)CC2)C1)C(C)C ; N#Cc1ccc(Cl)nc1 ; CCN(C(=O)c1cc(F)ccc1Oc1nncnc1N1CC2(CCN(CC3CCN(c4ccc(C#N)cn4)CC3)CC2)C1)C(C)C ; CCN(C(=O)c1cc(F)ccc1Oc1nncnc1N1CC2(CCN(CC3CCN(c4ccc(C(N)=O)cn4)CC3)CC2)C1)C(C)C ; "HCOOH"]

### (A) 2-((5-(7-((1-(5-cyanopyridin-2-yl)piperidin-4-yl)methyl)-2,7-diazaspiro[3.5]nonan-2-yl)-1,2,4-triazin-6-yl)oxy)-N-ethyl-5-fluoro-N-isopropylbenzamide

To a reaction flask were added successively intermediate 10-1 (80 mg, 0.15 mmol), 6-chloronicotinonitrile (41 mg, 0.30 mmol), potassium carbonate (52 mg, 0.38 mmol) and DMSO (3 ml), and the mixture was heated to 60° C. and reacted for 3 hours. The reaction solution was purified with flash column chromatography (eluted with a gradient of water/methanol=100:0-0:100) to obtain 72 mg of white solid. MS (m/z): 628.3 [M+H]⁺.

### (B) 6-(4-((2-(6-(2-(ethyl(isopropyl)carbamoyl)-4-fluorophenoxy)-1,2,4-triazin-5-yl)-2,7-diazaspiro[3.5]nonan-7-yl)methyl)piperidin-1-yl)nicotinamide formate

To a reaction flask were added successively 2-((5-(7-((1-(5-cyanopyridin-2-yl)piperidin-4-yl)methyl)-2,7-diazaspiro[3.5]nonan-2-yl)-1,2,4-triazin-6-yl)oxy)-N-ethyl-5-fluoro-N-isopropylbenzamide (72 mg, 0.12 mmol), potassium carbonate (30 mg, 0.22 mmol) and DMSO (3 ml), hydrogen peroxide (0.1 ml) was added dropwise, and the mixture was reacted at room temperature for 3 hours. The reaction solution was quenched with a saturated aqueous ammonium chloride solution (5 ml), extracted three times with ethyl acetate, washed with a saturated aqueous sodium bisulfate solution and saturated brine respectively, dried over anhydrous sodium sulfate, and concentrated to obtain a residue, which was purified with flash column chromatography (eluted with a gradient of water/methanol=100:0-0:100) to obtain 32.5 mg of light yellow solid. MS (m/z): 646.4 [M+H]⁺.

¹H NMR (400 MHz, CD₃OD) δ 8.62-8.59 (m, 1H), 8.41 (s, 1H), 8.38 (s, 1H), 7.96-7.93 (m, 1H), 7.42-7.39 (m, 1H), 7.32-7.19 (m, 2H), 6.84-6.81 (m, 1H), 4.49-4.45 (m, 4H), 4.03-4.00 (m, 2H), 3.88-3.75 (m, 1H), 3.49-3.45 (m, 1H), 3.25-3.15 (m, 5H), 2.99-2.95 (m, 2H), 2.91-2.87 (m, 2H), 2.15-2.09 (m, 5H), 1.89-1.85 (m, 2H), 1.29-1.25 (m, 2H), 1.20-1.06 (m, 7H), 0.81-0.76 (m, 2H).

### Compound 150: 4-(4-((2-(6-(2-(ethyl(isopropyl)carbamoyl)-4-fluorophenoxy)-1,2,4-triazin-5-yl)-2,7-diazaspiro[3.5]nonan-7-yl)methyl)piperidin-1-yl)nicotinamide

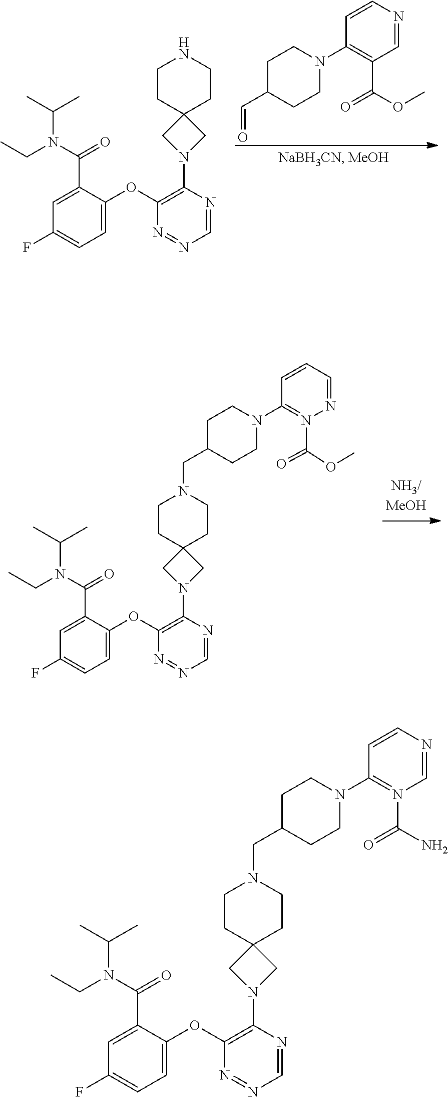

### (A) methyl 4-(4-((2-(6-(2-(ethyl(isopropyl)carbamoyl)-4-fluorophenoxy)-1,2,4-triazin-5-yl)-2,7-diazaspiro[3.5]nonan-7-yl)methyl)piperidin-1-yl)nicotinate

To a reaction flask were added successively intermediate 7-1 (68 mg, 0.16 mmol), intermediate 23 (60 mg, 0.24 mmol), methanol (5 ml) and sodium cyanoborohydride (20 mg, 0.32 mmol), and the mixture was reacted at room temperature for 2 hours. The reaction solution was quenched with a saturated aqueous ammonium chloride solution (2 ml), extracted three times with dichloromethane, dried over anhydrous sodium sulfate, and concentrated to obtain a residue, which was purified with flash column chromatography (eluted with a gradient of water/methanol=100:0-0:100) to obtain 72 mg of yellow solid. MS (m/z): 661.4 [M+H]⁺.

### (B) 4-(4-((2-(6-(2-(ethyl(isopropyl)carbamoyl)-4-fluorophenoxy)-1,2,4-triazin-5-yl)-2,7-diazaspiro[3.5]nonan-7-yl)methyl)piperidin-1-yl)nicotinamide

To a sealed tube were added successively methyl 4-(4-((2-(6-(2-(ethyl(isopropyl) carbamoyl)-4-fluorophenoxy)-1,2,4-triazin-5-yl)-2,7-diazaspiro[3.5]nonan-7-yl)methyl) piperidin-1-yl)nicotinate (72 mg, 0.11 mmol) and 7 mole/liter ammonia in methanol (8 ml, 0.56 mmol), and the mixture was heated to 80° C. and reacted for 40 hours. After the reaction was completed, the reaction solution was concentrated to obtain a crude product, which was purified with flash column chromatography (eluted with a gradient of water/methanol=100:0-0:100) to obtain 32.5 mg of light yellow solid. MS (m/z): 646.4 [M+H]⁺.

¹H NMR (400 MHz, CD₃OD) δ 8.41 (s, 1H), 8.36 (s, 1H), 8.26-8.23 (m, 1H), 7.39-7.36 (m, 1H), 7.26-7.23 (m, 1H), 7.18-7.11 (m, 1H), 6.98-6.94 (m, 1H), 4.35-4.30 (m, 2H), 3.97-3.79 (m, 2H), 3.59-3.55 (m, 2H), 3.13-3.09 (m, 1H), 2.92-2.88 (m, 2H), 2.43-2.38 (m, 4H), 2.24-2.21 (m, 2H), 1.90-1.82 (m, 6H), 1.78-1.75 (m, 1H), 1.38-1.32 (m, 4H), 1.21-0.97 (m, 7H), 0.89-0.85 (m, 2H).

The following compounds were prepared according to the preparation procedure of compound 150 using corresponding intermediates and reagents under appropriate conditions that will be recognized by those skilled in the art.

| | | MS | | Intermediate |
|---|---|---|---|---|
| Compound | Structural formula | (M + H)⁺ | ¹HNMR | used |
| 151 | 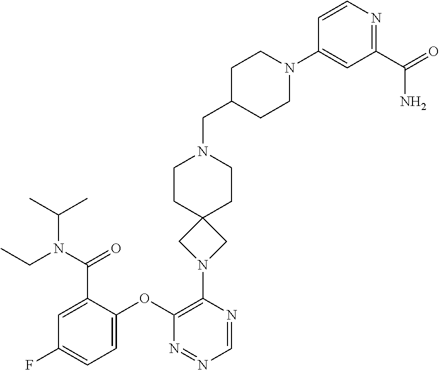 [SMILES: CCN(C(=O)c1cc(F)ccc1Oc1nncnc1N1CC2(CCN(CC3CCN(c4ccnc(C(N)=O)c4)CC3)CC2)C1)C(C)C] | 646.4 | ¹H NMR (400 MHz, CD₃OD) δ 8.39- 8.36 (m, 1H), 8.18-8.15 (m, 1H), 7.55- 7.51 (m, 1H), 7.42-7.40 (m, 1H), 7.28- 7.25 (m, 1H), 7.22-7.19 (m, 1H), 6.92- 6.88 (m, 1H), 4.38-4.35 (m, 2H), 4.07- 3.99 (m, 2H), 3.94-3.90 (m, 2H), 3.82- 3.79 (m, 1H), 3.56-3.44 (m, 1H), 3.27- 3.17 (m, 1H), 2.94-2.91 (m, 2H), 2.46- 2.40 (m, 4H), 2.22-2.18 (m, 2H), 1.89- 1.83 (m, 7H), 1.25-1.02 (m, 9H), 0.83- 0.80 (m, 2H). | 7-1 and 24- 13 |

### Compound 152: 5-((2-(6-(2-(ethyl(isopropyl)carbamoyl)-4-fluorophenoxy)-1,2,4-triazin-5-yl)-2,7-diazaspiro[3.5]nonan-7-yl)methyl)-1-(2-hydroxyethyl)-N,4-dimethyl-1H-indole-2-carboxamide

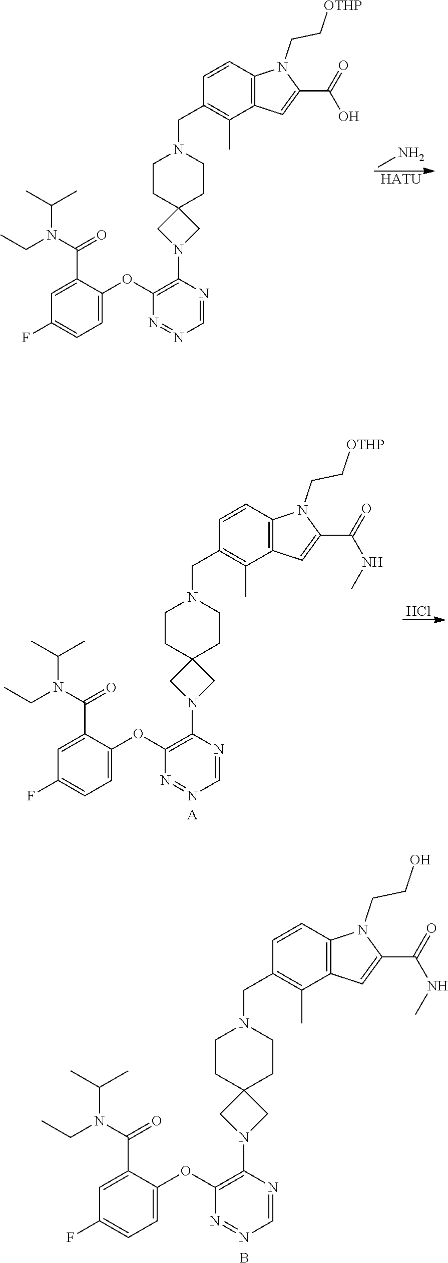

### (A) 5-((2-(6-(2-(ethyl(isopropyl)carbamoyl)-4-fluorophenoxy)-1,2,4-triazin-5-yl)-2,7-diazaspiro[3.5]nonan-7-yl)methyl)-N,4-dimethyl-1-(2-((tetrahydro-2H-pyran-2-yl)oxy)ethyl)-1H-indole-2-carboxamide

To a reaction flask were added successively 5-((2-(6-(2-(ethyl(isopropyl)carbamoyl)-4-fluorophenoxy)-1,2,4-triazin-5-yl)-2,7-diazaspiro[3.5]nonan-7-yl)methyl)-4-methyl-1-(2-((tetrahydro-2H-pyran-2-yl)oxy)ethyl)-1H-indole-2-carboxylic acid (30 mg, 0.04 mmol; prepared from intermediate 7-1 and intermediate 14 according to the preparation procedure of compound 57), methanamine hydrochloride (8 mg, 0.12 mmol), HATU (23 mg, 0.06 mmol), N,N-diisopropylethylamine (21 mg, 0.16 mmol) and dichloromethane (5 ml), and the mixture was reacted at room temperature for 16 hours. After the reaction was completed, the reaction solution was concentrated, and the residue was purified with flash column chromatography (eluted with a gradient of water/methanol=100:0-0:100) to obtain 20 mg of solid. MS (m/z): 757.4 [M+H]⁺.

### (B) 5-((2-(6-(2-(ethyl(isopropyl)carbamoyl)-4-fluorophenoxy)-1,2,4-triazin-5-yl)-2,7-diazaspiro[3.5]nonan-7-yl)methyl)-1-(2-hydroxyethyl)-N,4-dimethyl-1H-indole-2-carboxamide

In a reaction flask, 5-((2-(6-(2-(ethyl(isopropyl)carbamoyl)-4-fluorophenoxy)-1,2,4-triazin-5-yl)-2,7-diazaspiro[3.5]nonan-7-yl)methyl)-N,4-dimethyl-1-(2-((tetrahydro-2H-pyran-2-yl)oxy)ethyl)-1H-indole-2-carboxamide (20 mg, 0.026 mmol) was dissolved in methanol (5 ml), two drops of concentrated hydrochloric acid were added, and the mixture was reacted at room temperature for 0.5 hours. After the reaction was completed, the reaction solution was concentrated, and the residue was purified with flash column chromatography (eluted with a gradient of water/methanol=100:0-0:100) to obtain 11 mg of white solid. MS (m/z): 673.4 [M+H]⁺.

¹H NMR (400 MHz, DMSO-d6) δ 8.43 (s, 1H), 7.42 (dd, J=6.4, 3.6 Hz, 1H), 7.36-7.31 (m, 2H), 7.28 (d, J=8.5 Hz, 1H), 7.11-7.07 (m, 2H), 4.52 (t, J=5.8 Hz, 2H), 4.20 (s, 2H), 3.79 (s, 2H), 3.63 (t, J=5.5 Hz, 2H), 3.58 (d, J=6.7 Hz, 1H), 3.47 (s, 3H), 3.06-3.00 (m, 3H), 2.75 (d, J=4.4 Hz, 3H), 2.45 (s, 3H), 2.41-2.24 (m, 3H), 1.70 (s, 4H), 1.07-0.97 (m, 6H), 0.91-0.67 (in, 3H).

The following compounds were prepared according to the preparation procedure of compound 152 using corresponding intermediates and reagents under appropriate

| | | MS | | Intermediate |
|---|---|---|---|---|
| Compound | Structural formula | (M + H)⁺ | ¹HNMR | used |
| 153 | 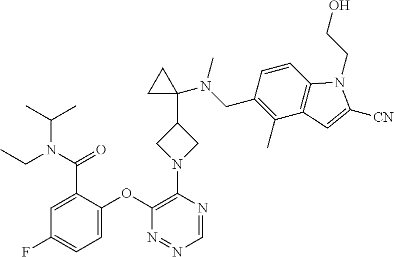 [SMILES: CCN(C(=O)c1cc(F)ccc1Oc1nncnc1N1CC(C2(N(C)Cc3ccc4c(cc(C#N)n4CCO)c3C)CC2)C1)C(C)C] | 641.6 | ¹H NMR (400 MHz, CD₃OD) δ 8.37 (s, 1H), 7.42-7.33 (m, 1H), 7.29-7.25 (m, 4H), 7.21-7.18 (m, 1H), 4.63-4.58 (m, 4H), 4.39-4.35 (m, 2H), 4.30-4.25 (m, 2H), 3.87-3.83 (m, 2H), 3.81-3.76 (m, 3H), 2.49 (s, 3H), 2.15 (s, 3H), 1.23- 1.04 (m, 8H), 0.83-0.77 (m, 6H). | 7-18 and 13- 1 |
| 154 | 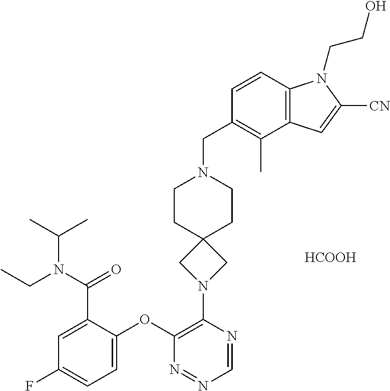 [SMILES: CCN(C(=O)c1cc(F)ccc1Oc1nncnc1N1CC2(CCN(Cc3ccc4c(cc(C#N)n4CCO)c3C)CC2)C1)C(C)C ; "HCOOH"] | 641.4 | ¹H NMR (400 MHz, CD₃OD) δ 8.40 (s, 2H), 7.53-7.35 (m, 4H), 7.32-7.17 (m, 2H), 4.45-4.40 (m, 4H), 4.28 (s, 2H), 4.03-3.98 (m, 2H), 3.89-3.85 (m, 2H), 3.83-3.74 (m, 1H), 3.48-3.45 (m, 1H), 3.20-3.12 (m, 5H), 2.62 (s, 3H), 2.09- 2.03 (m, 4H), 1.24-0.94 (m, 7H), 0.81- 0.75 (m, 2H). | 7-1 and 13-1 |
| 155 | 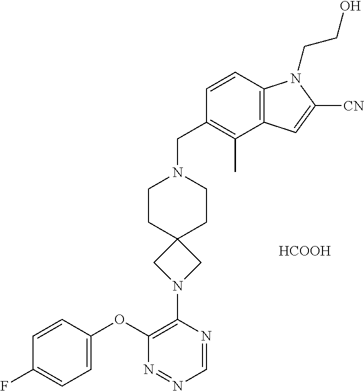 [SMILES: Cc1c(CN2CCC3(CC2)CN(c2ncnnc2Oc2ccc(F)cc2)C3)ccc2c1cc(C#N)n2CCO ; "HCOOH"] | 528.4 | ¹H NMR (400 MHz, CD₃OD) δ 8.44 (s, 1H), 8.37 (s, 1H), 7.45 (s, 2H), 7.38 (s, 1H), 7.29-7.18 (m, 2H), 7.17-7.12 (m, 2H), 4.49 (s, 2H), 4.41 (t, J = 5.2 Hz, 2H), 4.23 (s, 2H), 4.01 (s, 2H), 3.86 (t, J = 5.2 Hz, 2H), 3.14-3.06 (m, 4H), 2.61 (s, 3H), 2.10-2.04 (m, 4H). | 7-7 and 13-1 |
| 156 | 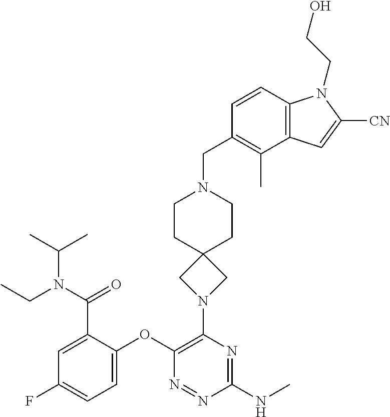 [SMILES: CCN(C(=O)c1cc(F)ccc1Oc1nnc(NC)nc1N1CC2(CCN(Cc3ccc4c(cc(C#N)n4CCO)c3C)CC2)C1)C(C)C] | 670.6 | ¹H NMR (400 MHz, CD₃OD) δ 7.31 (d, J = 4.9 Hz, 3H), 7.29-7.22 (m, 1H), 7.21-7.11 (m, 2H), 4.38 (t, J = 5.4 Hz, 2H), 4.32-4.09 (m, 2H), 3.91-3.78 (m, 4H), 3.74 (s, 1H), 3.57 (s, 2H), 3.55-3.45 (m, 1H), 3.28-3.20 (m, 1H), 2.94 (s, 1H), 2.80 (s, 2H), 2.53 (s, 3H), 2.55-2.30 (br, 3H), 1.83-1.75 (br, 4H), 1.25-1.02 (m, 7H), 0.93-0.86 (m, 2H). | 8 and 13-1 |
| 157 | 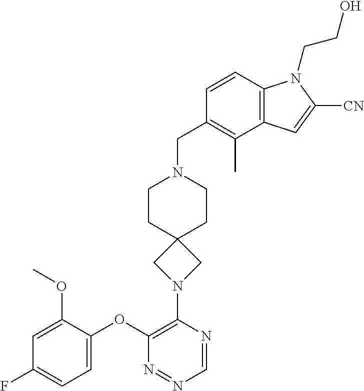 [SMILES: COc1cc(F)ccc1Oc1nncnc1N1CC2(CCN(Cc3ccc4c(cc(C#N)n4CCO)c3C)CC2)C1] | 558.3 | ¹H NMR (400 MHz, CD₃OD) δ 8.85 (d, J = 7.9 Hz, 1H), 7.62-7.50 (m, 2H), 7.45-7.72 (m, 1H), 7.36-7.33 (m, 1H), 7.04-7.00 (m, 1H), 6.83-6.72 (m, 1H), 4.74 (s, 1H), 4.53 (s, 1H), 4.54-4.38 (m, 5H), 4.30-4.27 (m, 1H), 3.89-3.86 (m, 2H), 3.83 (d, J = 7.4 Hz, 3H), 3.55- 3.50 (m, 2H), 3.30-3.16 (m, 2H), 2.67 (d, J = 14.3 Hz, 3H), 2.39-2.34 (m, 2H), 2.19-2.14 (m, 2H). | 7-9 and 13-1 |
| 158 | 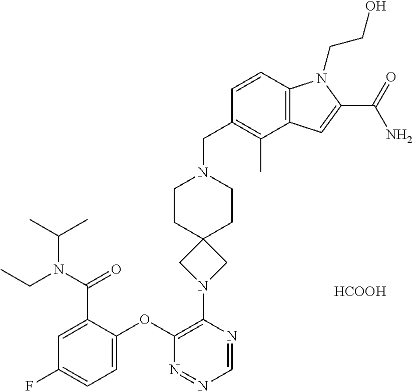 [SMILES: CCN(C(=O)c1cc(F)ccc1Oc1nncnc1N1CC2(CCN(Cc3ccc4c(cc(C(N)=O)n4CCO)c3C)CC2)C1)C(C)C ; "HCOOH"] | 659.4 | ¹H NMR (400 MHz, DMSO-d6) δ 8.43 (d, J = 0.4 Hz, 1H), 7.92 (s, 1H), 7.43 (dd, J = 9.6, 4.7 Hz, 1H), 7.35-7.27 (m, 4H), 7.19 (s, 1H), 7.11 (d, J = 8.6 Hz, 1H), 4.54 (t, J = 5.9 Hz, 2H), 4.20 (s, 2H), 3.79 (s, 2H), 3.62 (d, J = 5.7 Hz, 2H), 3.58 (d, J = 6.8 Hz, 1H), 3.49 (s, 3H), 3.15-3.14 (m, 1H), 3.10-3.01 (m, 2H), 2.45 (s, 3H), 2.30 (s, 3H), 1.70 (s, 4H), 1.07-0.96 (m, 6H), 0.92-0.66 (m, 3H). | 7-1 and 15 |
| 159 | 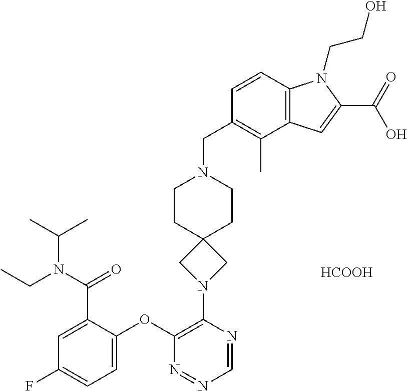 [SMILES: CCN(C(=O)c1cc(F)ccc1Oc1nncnc1N1CC2(CCN(Cc3ccc4c(cc(C(=O)O)n4CCO)c3C)CC2)C1)C(C)C ; "HCOOH"] | 660.4 | ¹H NMR (400 MHz, DMSO-d6) δ 8.43 (s, 1H), 7.45-7.39 (m, 1H), 7.35-7.28 (m, 3H), 7.19-7.13 (m, 2H), 4.55 (t, J = 5.6 Hz, 2H), 4.20 (s, 2H), 3.79 (s, 2H), 3.63 (d, J = 5.2 Hz, 2H), 3.58 (d, J = 6.5 Hz, 1H), 3.49 (s, 3H), 3.16-2.93 (m, 3H), 2.45 (s, 3H), 2.40-2.15 (m, 3H), 1.70 (s, 4H), 1.08-1.01 (m, 6H), 0.92- 0.67 (m, 3H). | 7-1 and 14 |
| 160 | 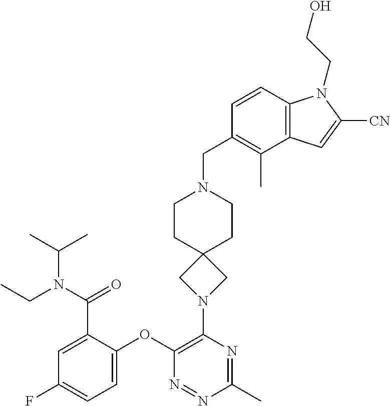 [SMILES: CCN(C(=O)c1cc(F)ccc1Oc1nnc(C)nc1N1CC2(CCN(Cc3ccc4c(cc(C#N)n4CCO)c3C)CC2)C1)C(C)C] | 655.6 | ¹H NMR (400 MHz, CD₃OD) δ 8.38 (s, 1H), 7.48-7.44 (m, 2H), 7.41 (s, 1H), 7.39-7.34 (m, 1H), 7.30-7.17 (m, 2H), 4.42 (t, J = 5.2 Hz, 4H), 4.28 (s, 2H), 4.07-3.95 (m, 2H), 3.87 (t, J = 5.3 Hz, 2H), 3.84-3.75 (m, 1H), 3.53-3.38 (m, 1H), 3.26-3.02 (m, 5H), 2.62 (s, 3H), 2.37 (s, 3H), 2.12-2.00 (br, 4H), 1.19- 1.03 (m, 7H), 0.79 (d, J = 6.2 Hz, 2H). | 9 and 13-1 |
| 161 | 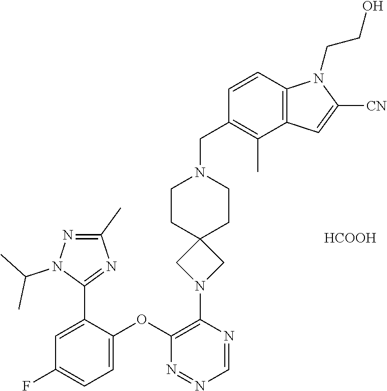 [SMILES: Cc1nc(-c2cc(F)ccc2Oc2nncnc2N2CC3(CCN(Cc4ccc5c(cc(C#N)n5CCO)c4C)CC3)C2)n(C(C)C)n1 ; "HCOOH"] | 651.6 | ¹H NMR (400 MHz, CD₃OD) δ 8.42 (s, 1H), 8.33 (s, 1H), 7.53-7.35 (m, 6H), 4.43-4.38 (m, 3H), 4.27 (s, 2H), 4.21 (s, 2H), 3.97 (s, 2H), 3.87 (t, J = 5.3 Hz, 2H), 3.10-3.05 (m, 4H), 2.64 (s, 3H), 2.32 (s, 3H), 2.05-2.00 (m, 4H), 1.31 (d, J = 6.6 Hz, 6H). | 7-13 and 13- 1 |
| 162 | 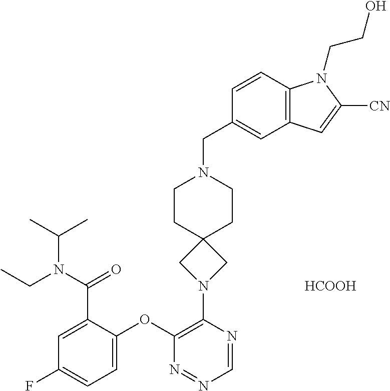 [SMILES: CCN(C(=O)c1cc(F)ccc1Oc1nncnc1N1CC2(CCN(Cc3ccc4c(c3)cc(C#N)n4CCO)CC2)C1)C(C)C ; "HCOOH"] | 627.3 | ¹H NMR (400 MHz, DMSO-d6) δ 8.43 (d, J = 0.6 Hz, 1H), 7.55 (d, J = 9.8 Hz, 2H), 7.40 (d, J = 4.4 Hz, 1H), 7.35-7.30 (m, 4H), 4.33 (t, J = 5.2 Hz, 2H), 4.18 (s, 2H), 3.78 (s, 2H), 3.68 (d, J = 5.2 Hz, 2H), 3.59 (d, J = 6.6 Hz, 1H), 3.54 (s, 3H), 3.08-2.98 (m, 2H), 2.38-2.30 (m, 4H), 1.73 (s, 4H), 1.06-0.93 (m, 6H), 0.90-0.64 (m, 3H). | 7-1 and 13-2 |
| 163 | 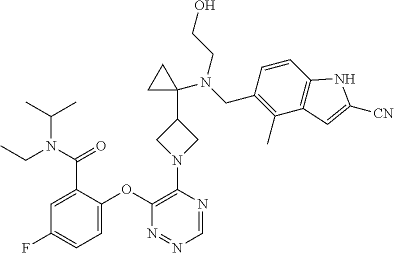 [SMILES: CCN(C(=O)c1cc(F)ccc1Oc1nncnc1N1CC(C2(N(CCO)Cc3ccc4[nH]c(C#N)cc4c3C)CC2)C1)C(C)C] | 627.8 | ¹H NMR (400 MHz, CD₃OD) δ 8.38 (s, 1H), 7.45-7.34 (m, 1H), 7.32-7.13 (m, 5H), 4.79-4.76 (m, 1H), 4.37-4.21 (m, 2H), 3.83-3.78 (m, 5H), 3.51-3.48 (m, 1H), 3.15 (t, J = 6.7 Hz, 2H), 2.74 (t, J = 6.7 Hz, 2H), 2.51 (s, 3H), 1.24-1.07 (m, 6H), 0.94-0.76 (m, 8H). | 7-18 |

### Compound 164: 2-((5-(7-((1-(6-aminopyridin-2-yl)piperidin-4-yl)methyl)-2,7-diazaspiro[3.5]nonan-2-yl)-1,2,4-triazin-6-yl)oxy)-N-ethyl-5-fluoro-N-isopropylbenzamide

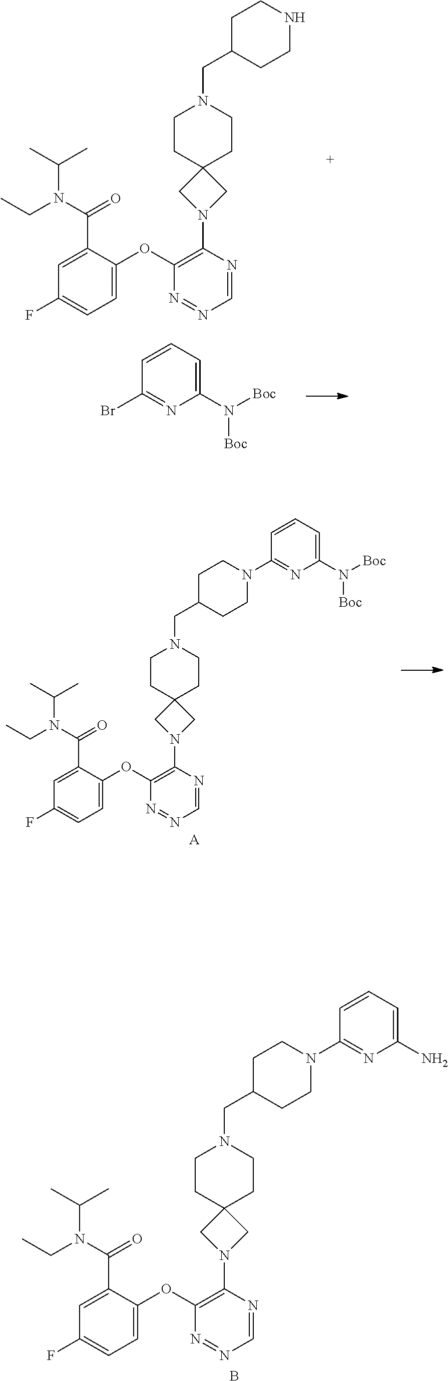

### (A) tert-butyl (tert-butoxycarbonyl)(6-(4-((2-(6-(2-(ethyl(isopropyl) carbamoyl)-4-fluorophenoxy)-1,2,4-triazin-5-yl)-2,7-diazaspiro[3.5]nonan-7-yl)methyl)piperidin-1-yl)pyridin-2-yl)carbamate

To a reaction flask were added successively intermediate 10-1 (50 mg, 0.10 mmol), tert-butyl (6-bromopyridin-2-yl)(tert-butoxycarbonyl)carbamate (74 mg, 0.20 mmol), Pd₂(dba)₃ (9 mg, 0.01 mmol), Xantphos (12 mg, 0.02 mmol), cesium carbonate (65 mg, 0.20 mmol) and 1,4-dioxane (5 ml), and the mixture was heated to 100° C. and reacted for hours under nitrogen protection. After the reaction was completed, the reaction solution was concentrated, and the residue was purified with flash column chromatography (eluted with a gradient of water/methanol=100:0-0:100) to obtain 14 mg of yellow solid. MS (m/z): 818.5 [M+H]⁺.

### (B) 2-((5-(7-((1-(6-aminopyridin-2-yl)piperidin-4-yl)methyl)-2,7-diazaspiro[3.5]nonan-2-yl)-1,2,4-triazin-6-yl)oxy)-N-ethyl-5-fluoro-N-isopropylbenzamide

To a reaction flask were added successively tert-butyl (tert-butoxycarbonyl)(6-(4-((2-(6-(2-(ethyl(isopropyl)carbamoyl)-4-fluorophenoxy)-1,2,4-triazin-5-yl)-2,7-diazaspiro[3.5]nonan-7-yl)methyl)piperidin-1-yl)pyridin-2-yl)carbamate (14 mg, 0.017 mmol), concentrated hydrochloric acid (0.25 ml) and methanol (2 ml), and the mixture was reacted at room temperature for 30 minutes. The reaction solution was concentrated, and the residue was purified with flash column chromatography (eluted with a gradient of water/methanol=100:0-0:100) to obtain 4.5 mg of yellow solid. MS (m/z): 618.4 [M+H]⁺.

¹H NMR (400 MHz, a mixture of CDCl₃ and CD₃OD) δ 8.36 (s, 1H), 7.38-7.35 (m, 1H), 7.26-7.19 (m, 2H), 7.13-7.10 (m, 1H), 6.00-5.97 (m, 1H), 5.89-5.86 (m, 1H), 4.38-4.35 (m, 2H), 4.15-4.12 (m, 2H), 3.93-3.90 (m, 2H), 3.86-3.76 (m, 1H), 2.72-2.68 (m, 2H), 2.47-2.41 (m, 3H), 2.22-2.17 (m, 2H), 1.90-1.85 (m, 5H), 1.81-1.77 (m, 3H), 1.25-1.02 (m, 11H), 0.82-0.78 (m, 2H).

### Compound 165: N-ethyl-5-fluoro-N-isopropyl-2-((5-(7-((1-(pyrimidin-4-yl)piperidin-4-yl)methyl)-2,7-diazaspiro[3.5]nonan-2-yl)-1,2,4-triazin-6-yl)oxy)benzamide

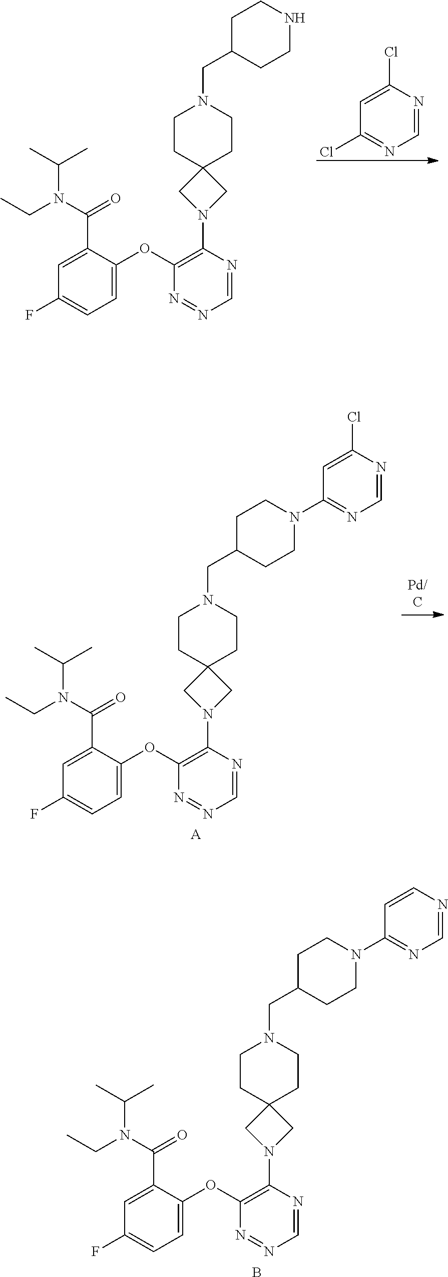

### (A) 2-((5-(7-((1-(6-chloropyrimidin-4-yl)piperidin-4-yl)methyl)-2,7-diazaspiro[3.5]nonan-2-yl)-1,2,4-triazin-6-yl)oxy)-N-ethyl-5-fluoro-N-isopropylbenzamide

To a reaction flask were added intermediate 10-1 (50 mg, 0.095 mmol), 4,6-dichloropyrimidine (14 mg, 0.094 mmol), triethylamine (19 mg, 0.1902 mmol) and dichloromethane (10 ml), and the mixture was stirred and reacted for 15 hours at room temperature. After the reaction was completed, the reaction solution was concentrated, and the residue was purified with flash column chromatography (eluted with a gradient of water/methanol=100:0-0:100) to obtain 30 mg of yellow solid. MS (m/z): 638.3 [M+H]⁺.

### (B) N-ethyl-5-fluoro-N-isopropyl-2-((5-(7-((1-(pyrimidin-4-yl)piperidin-4-yl)methyl)-2,7-diazaspiro[3.5]nonan-2-yl)-1,2,4-triazin-6-yl)oxy)benzamide

To a reaction flask were added 2-((5-(7-((1-(6-chloropyrimidin-4-yl)piperidin-4-yl)methyl)-2,7-diazaspiro[3.5]nonan-2-yl)-1,2,4-triazin-6-yl)oxy)-N-ethyl-5-fluoro-N-isopropylbenzamide (30 mg, 0.047 mmol), palladium carbon (6 mg) and methanol (10 ml), and the mixture was reacted under hydrogen at room temperature for 15 hours. After the reaction was completed, the reaction solution was filtered, the filtrate was concentrated, and the residue was purified with flash column chromatography (eluted with a gradient of water/methanol=100:0-0:100) to obtain 20 mg of white solid. MS (m/z): 604.4 [M+H]⁺.

¹H NMR (400 MHz, DMSO-d6) δ 8.43 (s, 2H), 8.14 (s, 1H), 8.10 (d, J=5.7 Hz, 1H), 7.46-7.36 (m, 1H), 7.36-7.27 (m, 2H), 6.77 (d, J=5.9 Hz, 1H), 4.42-4.30 (m, 2H), 4.19 (s, 2H), 3.82-3.73 (m, 2H), 3.63-3.55 (m, 1H), 3.42-3.30 (m, 1H), 3.10-2.92 (m, 1H), 2.84 (t, J=11.9 Hz, 2H), 2.44-2.15 (br, 4H), 2.10 (d, J=6.4 Hz, 2H), 1.85-1.65 (m, 7H), 1.21 (s, 1H), 1.11-0.91 (m, 8H), 0.74-0.63 (m, 2H).

The following compounds were prepared according to the preparation procedure of compound 165 using corresponding intermediates and reagents under appropriate conditions that will be recognized by those skilled in the art.

| | | MS | | Intermediate |
|---|---|---|---|---|
| Compound | Structural formula | (M + H)⁺ | ¹HNMR | used |
| 166 | 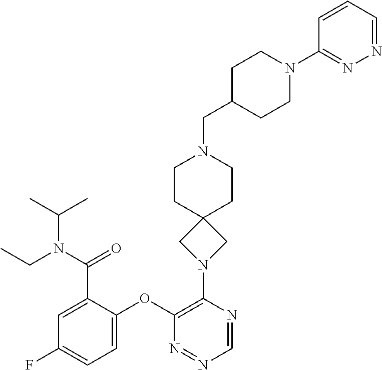 [SMILES: CCN(C(=O)c1cc(F)ccc1Oc1nncnc1N1CC2(CCN(CC3CCN(c4cccnn4)CC3)CC2)C1)C(C)C] | 604.4 | ¹H NMR (400 MHz, CD₃OD) δ 8.40- 8.38 (m, 1H), 8.36 (s, 1H), 7.39-7.36 (m, 1H), 7.34-7.31 (m, 1H), 7.28-7.20 (m, 1H), 7.20-7.11 (m, 2H), 4.36-4.33 (m, 4H), 3.89-3.84 (m, 3H), 3.13-3.10 (m, 1H), 3.01-2.89 (m, 2H), 2.44-2.40 (m, 4H), 2.23-2.20 (m, 2H), 1.89-1.83 (m, 8H), 1.26-1.03 (m, 9H), 0.89-0.85 (m, 2H). | 10-1 |
| 167 | 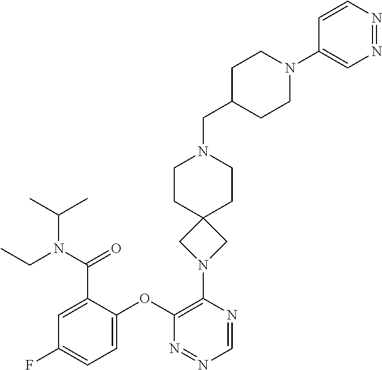 [SMILES: CCN(C(=O)c1cc(F)ccc1Oc1nncnc1N1CC2(CCN(CC3CCN(c4ccnnc4)CC3)CC2)C1)C(C)C] | 604.4 | ¹H NMR (400 MHz, CD₃OD) δ 8.74 (d, J = 3.3 Hz, 1H), 8.42 (d, J = 6.5 Hz, 1H), 8.29 (d, J = 1.1 Hz, 1H), 7.34-7.29 (m, 1H), 7.21-7.16 (m, 1H), 7.11 (dd, J = 7.7, 2.6 Hz, 1H), 6.86 (dd, J = 6.4, 3.1 Hz, 1H), 4.27 (d, J = 14.0 Hz, 2H), 3.99 (d, J = 13.3 Hz, 2H), 3.83 (d, J = 8.1 Hz, 2H), 3.71 (dt, J = 13.3, 6.7 Hz, 1H), 3.41 (dd, J = 13.6, 7.0 Hz, 1H), 2.89 (t, J = 12.1 Hz, 2H), 2.35 (s, 4H), 2.12 (d, J = 6.5 Hz, 2H), 1.84-1.75 (m, 8H), 1.15- 1.05 (m, 6H), 0.99-0.95 (m, 3H), 0.71 (d, J = 6.2 Hz, 2H). | 10-1 |
| 168 | 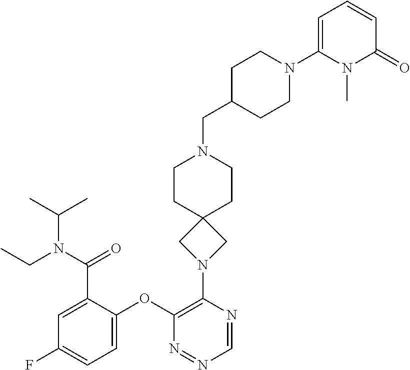 [SMILES: CCN(C(=O)c1cc(F)ccc1Oc1nncnc1N1CC2(CCN(CC3CCN(c4cccc(=O)n4C)CC3)CC2)C1)C(C)C] | 633.6 | ¹H NMR (400 MHz, CD₃OD) δ 8.38 (s, 1H), 7.50-7.37 (m, 2H), 7.32-7.18 (m, 2H), 6.25 (d, J = 8.2 Hz, 1H), 6.04 (d, J = 7.5 Hz, 1H), 4.42-4.22 (m, 2H), 3.97- 3.88 (m, 2H), 3.85-3.75 (m, 1H), 3.54 (s, 3H), 3.53-3.44 (m, 1H), 3.25-3.18 (m, 3H), 2.68 (t, J = 11.6 Hz, 2H), 2.56-2.31 (br, 4H), 2.26 (d, J = 7.0 Hz, 2H), 1.93- 1.90 (m, 1H), 1.90-1.85 (m, 5H), 1.80- 1.70 (m, 1H), 1.44-1.35 (m, 2H), 1.21 (d, J = 6.8 Hz, 2H), 1.18-1.12 (m, 5H), 1.06 (t, J = 7.1 Hz, 1H), 0.81 (d, J = 6.1 Hz, 2H). | 10-1 |

### Compound 169: N-ethyl-5-fluoro-N-isopropyl-2-((5-(7-((1-(pyrazolo[1,5-a]pyrimidine-3-carbonyl)piperidin-4-yl)methy)-2,7-diazaspiro[3.5]nonan-2-yl)-1,2,4-triazin-6-yl)oxy)benzamide

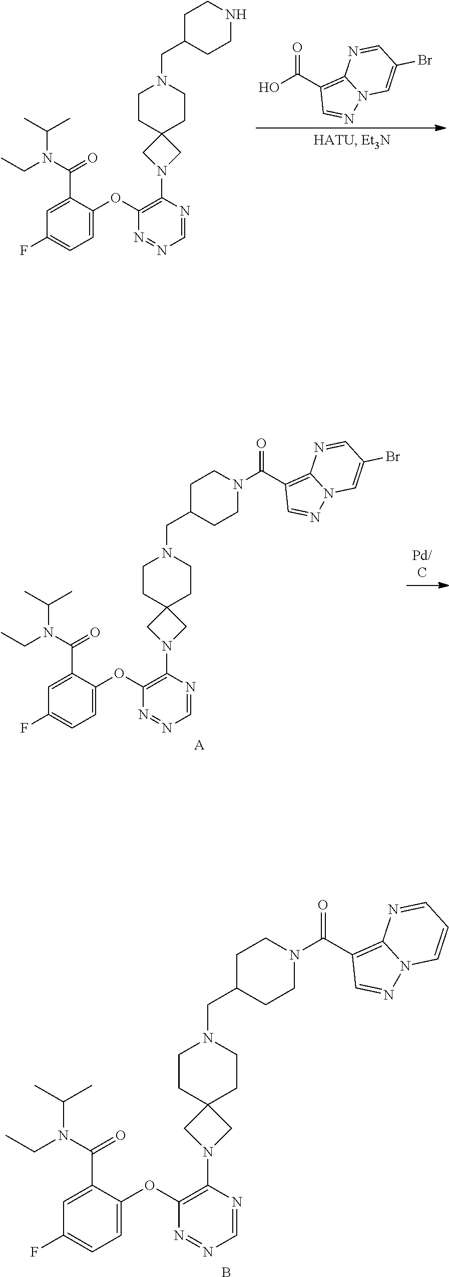

### (A) 2-((5-(7-((1-(6-bromopyrazolo[1,5-a]pyrimidine-3-carbonyl)piperidin-4-yl)methyl)-2,7-diazaspiro[3.5]nonan-2-yl)-1,2,4-triazin-6-yl)oxy)-N-ethyl-5-fluoro-N-isopropylbenzamide

To a reaction flask were added successively intermediate 10-1 (50 mg, 0.10 mmol), 6-bromopyrazolo[1,5-a]pyrimidine-3-carboxylic acid (48 mg, 0.20 mmol), HATU (49 mg, 0.13 mmol), triethylamine (30 mg, 0.30 mmol) and N,N-dimethylformamide (3 ml), and the mixture was reacted at room temperature for 1 hour. The reaction solution was purified with flash column chromatography (eluted with a gradient of water/methanol=100:0-0:100) to obtain 61 mg of light yellow solid. MS (m/z): 749.2 [M+H]⁺.

### (B) N-ethyl-5-fluoro-N-isopropyl-2-((5-(7-((1-(pyrazolo[1,5-a]pyrimidine-3-carbonyl)piperidin-4-yl)methyl)-2,7-diazaspiro[3.5]nonan-2-yl)-1,2,4-triazin-6-yl)oxy)benzamide

To a reaction flask were added successively 2-((5-(7-((1-(6-bromopyrazolo[1,5-a]pyrimidine-3-carbonyl)piperidin-4-yl)methyl)-2,7-diazaspiro[3.5]nonan-2-yl)-1,2,4-triazin-6-yl)oxy)-N-ethyl-5-fluoro-N-isopropylbenzamide (61 mg, 0.081 mmol), palladium carbon (6 mg) and methanol (6 ml), and the mixture was reacted for 2 hours under hydrogen. After the reaction was completed, the reaction solution was filtered, the filtrate was concentrated, and the residue was purified with flash column chromatography (eluted with a gradient of water/methanol=100:0-0:100) to obtain 16.5 mg of product. MS (m/z): 671.4 [M+H]⁺.

¹H NMR (400 MHz, CD₃OD) δ 9.02-8.99 (m, 1H), 8.67 (s, 1H), 8.41-8.38 (m, 2H), 7.40-7.37 (m, 1H), 7.35-7.19 (m, 2H), 7.15-7.13 (m, 1H), 4.70-4.68 (m, 1H), 4.46-4.43 (m, 2H), 4.12-4.08 (m, 3H), 3.82-3.79 (m, 1H), 3.48-3.45 (m, 1H), 3.16-3.11 (m, 6H), 2.92-2.86 (m, 3H), 2.13-2.08 (m, 5H), 1.86-1.82 (m, 2H), 1.33-1.30 (m, 2H), 1.26-0.99 (m, 7H), 0.82-0.77 (m, 2H).

### Compound 170: methyl 4-((2-(6-(2-(ethyl(isopropyl)carbamoyl)-4-fluorophenoxy)-1,2,4-triazin-5-yl)-2,7-diazaspiro[3.5]nonan-7-yl)methyl)piperidine-1-carboxylate

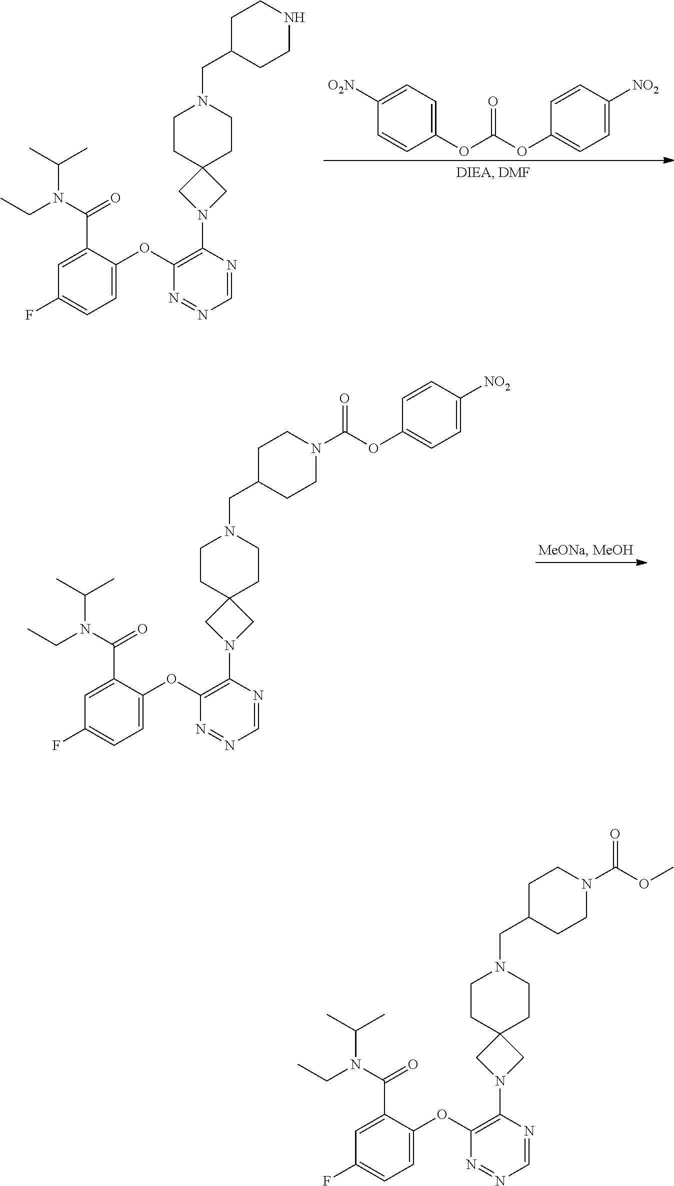 [SMILES: CCN(C(=O)c1cc(F)ccc1Oc1nncnc1N1CC2(CCN(CC3CCNCC3)CC2)C1)C(C)C ; O=C(Oc1ccc([N+](=O)[O-])cc1)Oc1ccc([N+](=O)[O-])cc1 ; CCN(C(=O)c1cc(F)ccc1Oc1nncnc1N1CC2(CCN(CC3CCN(C(=O)Oc4ccc([N+](=O)[O-])cc4)CC3)CC2)C1)C(C)C ; CCN(C(=O)c1cc(F)ccc1Oc1nncnc1N1CC2(CCN(CC3CCN(C(=O)OC)CC3)CC2)C1)C(C)C]

### (A) 4-nitrophenyl 4-((2-(6-(2-(ethyl(isopropyl)carbamoyl)-4-fluorophenoxy)-1,2,4-triazin-5-yl)-2,7-diazaspiro[3.5]nonan-7-yl)methyl)piperidine-1-carboxylate

To a reaction flask were added successively intermediate 10-1 (45 mg, 0.086 mmol), bis(4-nitrobenzene)carbonate (33 mg, 0.11 mmol), N,N-diisopropylethylamine (22 mg, 0.17 mmol) and N,N-dimethylformamide (5 ml), and the mixture was reacted at room temperature for 2 hours. After the reaction was completed, the reaction solution was concentrated, and the residue was purified with flash column chromatography (eluted with a gradient of water/methanol=100:0-0:100) to obtain 50 mg of solid. MS (m/z): 691.4 [M+H]⁺.

### (B) methyl 4-((2-(6-(2-(ethyl(isopropyl)carbamoyl)-4-fluorophenoxy)-1,2,4-triazin-5-yl)-2,7-diazaspiro[3.5]nonan-7-yl)methyl)piperidine-1-carboxylate

To a reaction flask were added successively 4-nitrophenyl 4-((2-(6-(2-(ethyl (isopropyl)carbamoyl)-4-fluorophenoxy)-1,2,4-triazin-5-yl)-2,7-diazaspiro[3.5]nonan-7-yl)methyl)piperidine-1-carboxylate (30 mg, 0.043 mmol), sodium methoxide (5 mg, 0.093 mmol) and methanol (5 ml), and the mixture was heated to 80° C. and reacted for 5 hours. After the reaction was completed, the reaction solution was concentrated, and the residue was purified with flash column chromatography (eluted with a gradient of water/methanol=100:0-0:100) to obtain 6 mg of white solid. MS (m/z): 584.4 [M+H]⁺.

¹H NMR (400 MHz, DMSO-d6) δ 8.42 (s, 1H), 7.43-7.38 (m, 1H), 7.32 (dd, J=9.9, 5.8 Hz, 2H), 4.17 (s, 2H), 3.90 (s, 2H), 3.76 (s, 2H), 3.59 (d, J=6.7 Hz, 1H), 3.53 (s, 3H), 3.03-2.97 (m, 1H), 2.71 (s, 2H), 2.30 (s, 4H), 2.03 (d, J=6.8 Hz, 2H), 1.72-1.61 (m, 8H), 1.09-0.99 (m, 6H), 0.96-0.92 (m, 3H), 0.68 (d, J=5.8 Hz, 2H).

### Compound 171: 2-((5-(7-(1-(1-(5,6-dimethoxypyridazin-3-yl)piperidin-4-yl)ethyl)-2,7-diazaspiro[3.5]nonan-2-yl)-1,2,4-triazin-6-yl)oxy)-N-ethyl-5-fluoro-N-isopropylbenzamide

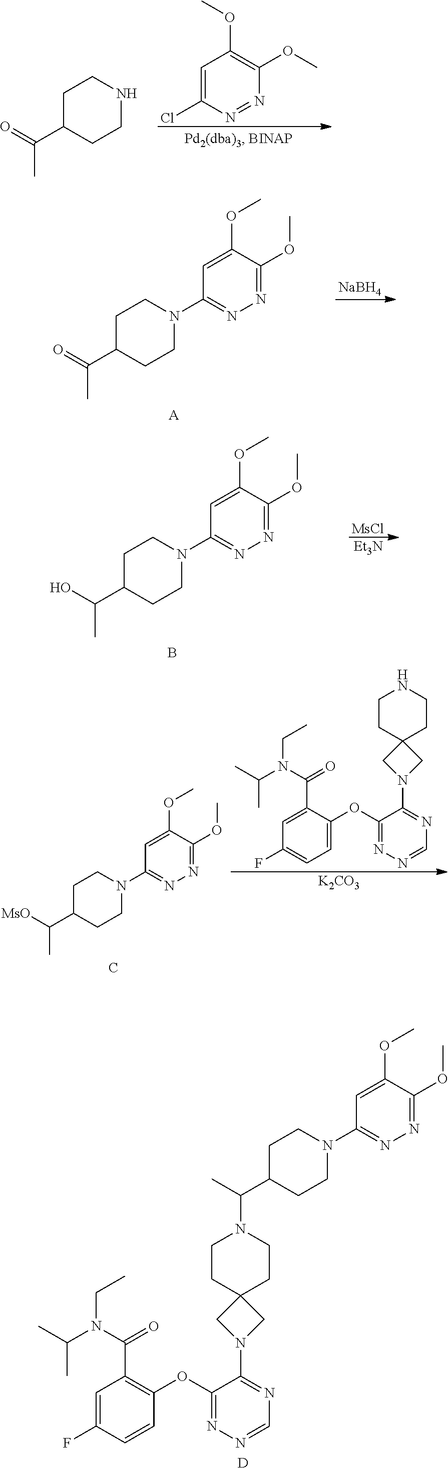

### (A) 1-(1-(5,6-dimethoxypyridazin-3-yl)piperidin-4-yl)ethan-1-one

To a reaction flask were added successively 6-chloro-3,4-dimethoxypyridazine (523 mg, 3 mmol), 1-(piperidin-4-yl)ethan-1-one (572 mg, 4.5 mmol), Pd₂(dba)₃ (137 mg, 0.15 mmol), BINAP (187 mg, 0.3 mmol), sodium methoxide (324 mg, 6 mmol) and toluene (30 ml), and the mixture was heated to 110° C. and reacted for 16 hours under nitrogen protection. After the reaction was completed, the reaction solution was concentrated, and the residue was purified with flash column chromatography (eluted with a gradient of water/methanol=100:0-0:100) to obtain 110 mg of solid. MS (m/z): 266.2 [M+H]⁺.

### (B) 1-(1-(5,6-dimethoxypyridazin-3-yl)piperidin-4-yl)ethan-1-ol

To a reaction flask were added successively 1-(1-(5,6-dimethoxypyridazin-3-yl)piperidin-4-yl)ethan-1-one (110 mg, 0.42 mmol) and methanol (10 ml), and the mixture was cooled to 0° C. Sodium borohydride (46 mg, 1.22 mmol) was added, and the mixture was reacted at room temperature for 1 hour. After the reaction was completed, the reaction solution was concentrated, and the residue was purified with flash column chromatography (eluted with a gradient of water/methanol=100:0-0:100) to obtain 80 mg of solid. MS (m/z): 268.4 [M+H]⁺.

### (C) 1-(1-(5,6-dimethoxypyridazin-3-yl)piperidin-4-yl)ethyl methanesulfonate

To a reaction flask were added successively 1-(1-(5,6-dimethoxypyridazin-3-yl)piperidin-4-yl)ethan-1-ol (80 mg, 0.3 mmol), triethylamine (1 ml) and dichloromethane (10 ml), and the mixture was cooled to 0° C. Methanesulfonyl chloride (52 mg, 0.45 mmol) was added, and the mixture was reacted at room temperature for 2 hours. After the reaction was completed, the reaction solution was concentrated, and the residue was purified with flash column chromatography (eluted with a gradient of water/methanol=100:0-0:100) to obtain 50 mg of solid. MS (m/z): 346.1 [M+H]⁺.

### (D) 2-((5-(7-(1-(1-(5,6-dimethoxypyridazin-3-yl)piperidin-4-yl)ethyl)-2,7-diazaspiro[3.5]nonan-2-yl)-1,2,4-triazin-6-yl)oxy)-N-ethyl-5-fluoro-N-isopropylbenzamide

To a reaction flask were added successively intermediate 7-1 (50 mg, 0.12 mmol), 1-(1-(5,6-dimethoxypyridazin-3-yl)piperidin-4-yl)ethyl methanesulfonate (50 mg, 0.15 mmol), potassium carbonate (33 mg, 0.24 mmol) and dioxane (10 ml), and the mixture was heated to 100° C. and reacted for 15 hours. After the reaction was completed, the reaction solution was concentrated, and the residue was purified with flash column chromatography (eluted with a gradient of water/methanol=100:0-0:100) to obtain 10 mg of white solid. MS (m/z): 678.4 [M+H]⁺.

¹H NMR (400 MHz, CD₃OD) δ 8.41 (s, 1H), 8.36 (s, 2H), 7.44-7.36 (m, 1H), 7.32-7.20 (m, 2H), 6.56 (s, 1H), 4.44 (s, 2H), 4.08-3.98 (m, 5H), 3.95 (s, 3H), 3.87-3.73 (m, 1H), 3.67-3.55 (m, 2H), 3.53-3.41 (m, 1H), 3.26-3.03 (m, 6H), 2.42-2.29 (m, 1H), 2.17-2.03 (m, 5H), 1.92-1.82 (m, 2H), 1.79-1.66 (m, 1H), 1.34-1.24 (m, 4H), 1.20-1.03 (m, 7H), 0.92-0.74 (m, 3H).

### Compound 172: N-ethyl-5-fluoro-N-isopropyl-2-((5-(7-(1-(pyridin-2-yl)piperidine-4-carbonyl)-2,7-diazaspiro[3.5]nonan-2-yl)-1,2,4-triazin-6-yl)oxy)benzamide

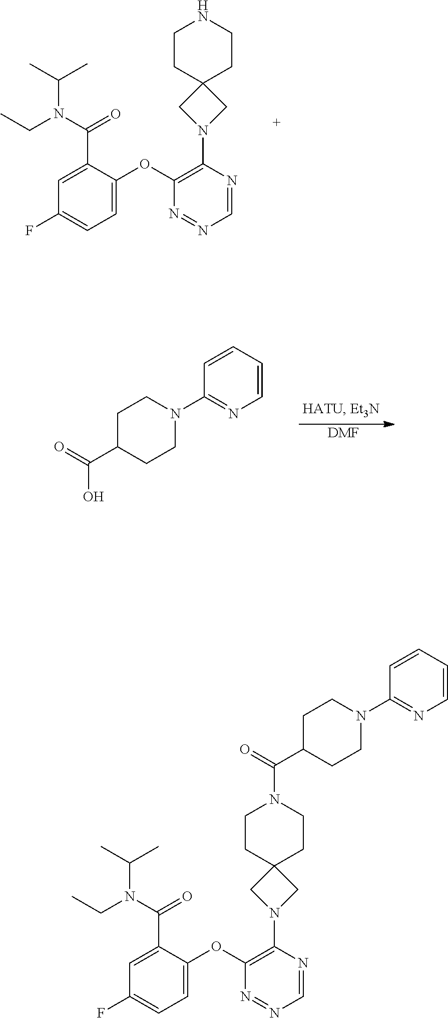

To a reaction flask were added successively intermediate 7-1 (50 mg, 0.12 mmol), intermediate 11 (24 mg, 0.12 mmol), HATU (46 mg, 0.12 mmol), triethylamine (36 mg, 0.36 mmol) and N,N-dimethylformamide (3 ml), and the mixture was reacted at room temperature for 1 hour. The reaction solution was purified with flash column chromatography (eluted with a gradient of water/methanol=100:0-0:100) to obtain 24.6 mg of light yellow solid. MS (m/z): 617.4 [M+H]⁺.

¹H NMR (400 MHz, CD₃OD) δ 8.38 (s, 1H), 7.43-7.40 (m, 1H), 7.33-7.23 (m, 1H), 7.21-7.18 (m, 1H), 6.68 (s, 1H), 4.39-4.36 (m, 2H), 4.17-4.06 (m, 2H), 3.95 (s, 3H), 3.94-3.89 (m, 2H), 3.88 (s, 3H), 3.83-3.80 (m, 1H), 3.26-3.14 (m, 1H), 2.88-2.85 (m, 2H), 2.46-2.41 (m, 3H), 2.24-2.20 (m, 2H), 1.88-1.83 (m, 7H), 1.37-1.00 (m, 11H), 0.83-0.79 (m, 2H).

### Compound 173: N-ethyl-5-fluoro-N-isopropyl-2-((5-(7-((4-(pyridin-2-yl)piperazin-1-yl)methyl)-2-azaspiro[3.5]nonan-2-yl)-1,2,4-triazin-6-yl)oxy)benzamide

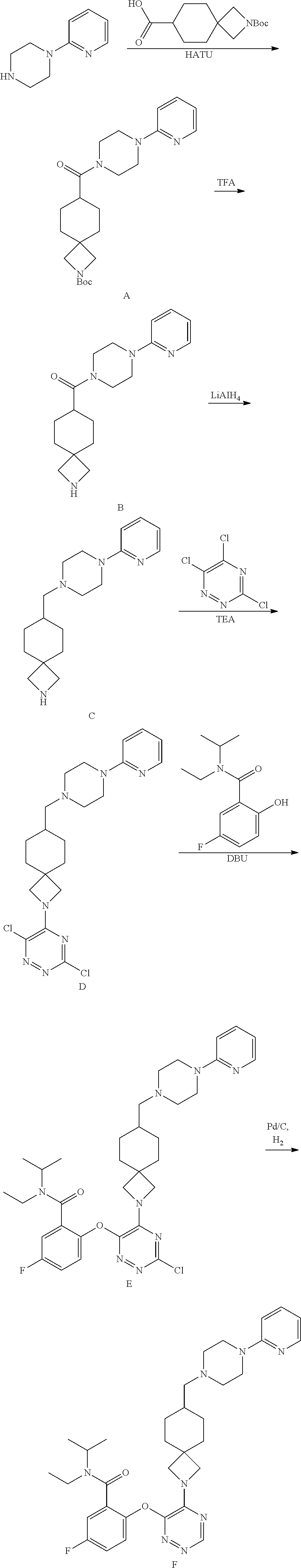

### (A) tert-butyl 7-(4-(pyridin-2-yl)piperazine-1-carbonyl)-2-azaspiro[3.5]nonane-2-carboxylate

To a reaction flask were added successively 1-(pyridine-2-yl)piperazine (480 mg, 2.9 mmol), 2-(tert-butoxycarbonyl)-2-azaspiro[3.5]nonane-7-carboxylic acid (792 mg, 2.9 mmol), HATU (1678 mg, 4.4 mmol), N,N-diisopropylethylamine (1140 mg, 8.8 mmol) and dichloromethane (10 ml), and the mixture was reacted at room temperature for 16 hours. After the reaction was completed, the reaction solution was concentrated, and the residue was purified with flash column chromatography (eluted with a gradient of water/methanol=100:0-0:100) to obtain 500 mg of solid. MS (m/z): 415.1 [M+H]⁺.

### (B) (4-(pyridin-2-yl)piperazin-1-yl)(2-azaspiro[3.5]nonan-7-yl)methanone

To a reaction flask were added successively tert-butyl 7-(4-(pyridin-2-yl)piperazine-1-carbonyl)-2-azaspiro[3.5]nonane-2-carboxylate (500 mg, 1.2 mmol), dichloromethane (5 ml) and trifluoroacetic acid (2 ml), and the mixture was reacted at room temperature for 16 hours. After the reaction was completed, the reaction solution was neutralized with an aqueous sodium bicarbonate solution and concentrated, and the residue was purified with flash column chromatography (eluted with a gradient of water/methanol=100:0-0:100) to obtain 300 mg of solid. MS (m/z): 315.2 [M+H]⁺.

### (C) 7-((4-(pyridin-2-yl)piperazin-1-yl)methyl)-2-azaspiro[3.5]nonane

To a reaction flask were added successively (4-(pyridin-2-yl)piperazin-1-yl)(2-azaspiro[3.5]nonan-7-yl)methanone (300 mg, 0.95 mmol), tetrahydrofuran (10 ml) and lithium aluminum hydride (72 mg, 1.90 mmol), and the mixture was heated to 70′T and reacted for 3 days. After the reaction was completed, the reaction solution was concentrated, and the residue was purified with flash column chromatography (eluted with a gradient of water/methanol=100:0-0:100) to obtain 150 mg of solid. MS (m/z): 301.2 [M+H]⁺.

### (D) 2-(3,6-dichloro-1,2,4-triazin-5-yl)-7-((4-(pyridin-2-yl)piperazin-1-yl)methyl)-2-azaspiro[3.5]nonane

To a reaction flask were added successively 7-((4-(pyridin-2-yl)piperazin-1-yl)methyl)-2-azaspiro[3.5]nonane (130 mg, 0.43 mmol), 3,5,6-trichloro-1,2,4-triazine (116 mg, 0.63 mmol), triethylamine (131 mg, 1.30 mmol) and dichloromethane (5 ml), and the mixture was reacted at room temperature for 3 hours. After the reaction was completed, the reaction solution was concentrated, and the residue was purified with flash column chromatography (eluted with a gradient of water/methanol=100:0-0:100) to obtain 120 mg of solid. MS (m/z): 448.2 [M+H]⁺.

### (E) 2-((3-chloro-5-(7-((4-(pyridin-2-yl)piperazin-1-yl)methyl)-2-azaspiro[3.5]nonan-2-yl)-1,2,4-triazin-6-yl)oxy)-N-ethyl-5-fluoro-N-isopropylbenzamide

To a reaction flask were added successively 2-(3,6-dichloro-1,2,4-triazin-5-yl)-7-((4-(pyridin-2-yl)piperazin-1-yl)methyl)-2-azaspiro[3.5]nonane (120 mg, 0.27 mmol), N-ethyl-5-fluoro-2-hydroxy-N-isopropylbenzamide (61 mg, 0.27 mmol), DBU (101 mg, 0.67 mmol) and tetrahydrofuran (5 ml), and the mixture was reacted at 40° C. for 16 hours. After the reaction was completed, the reaction solution was concentrated, and the residue was purified with flash column chromatography (eluted with a gradient of water/methanol=100:0-0:100) to obtain 30 mg of solid. MS (m/z): 637.4 [M+H]⁺.

### (F) N-ethyl-5-fluoro-N-isopropyl-2-((5-(7-((4-(pyridin-2-yl)piperazin-1-yl)methyl)-2-azaspiro[3.5]nonan-2-yl)-1,2,4-triazin-6-yl)oxy)benzamide

To a reaction flask were added successively 2-((3-chloro-5-(7-((4-(pyridin-2-yl)piperazin-1-yl)methyl)-2-azaspiro[3.5]nonan-2-yl)-1,2,4-triazin-6-yl)oxy)-N-ethyl-5-fluoro-N-isopropylbenzamide (20 mg, 0.03 mmol), palladium carbon (10 mg) and methanol (5 ml), and the mixture was reacted under hydrogen at room temperature for 5 hours. After the reaction was completed, the reaction solution was concentrated, and the residue was purified with flash column chromatography (eluted with a gradient of water/methanol=100:0-0:100) to obtain 6 mg of solid. MS (m/z): 603.4 [M+H]⁺.

¹H NMR (400 MHz, DMSO-d6) δ 8.41 (s, 1H), 8.06 (d, J=4.3 Hz, 1H), 7.48 (t, J=7.8 Hz, 1H), 7.41 (d, J=4.6 Hz, 1H), 7.32 (s, 2H), 6.76 (d, J=8.6 Hz, 1H), 6.61-6.57 (m, 1H), 4.17-4.13 (m, 2H), 3.79-3.71 (m, 2H), 3.62-3.54 (m, 1H), 3.41 (s, 4H), 3.07 (s, 1H), 2.36 (s, 4H), 2.08 (s, 2H), 1.85 (s, 2H), 1.70 (s, 2H), 1.46 (s, 4H), 1.01 (dd, J=14.0, 7.0 Hz, 6H), 0.88-0.84 (m, 3H), 0.67 (d, J=5.0 Hz, 2H).

### Compound 175: 2-((5-(7-((2-cyano-4-methyl-1H-indol-5-yl)methyl-d)-2,7-diazaspiro[3.5]nonan-2-yl)-1,2,4-triazin-6-yl)oxy)-N-ethyl-5-fluoro-N-isopropylbenzamide

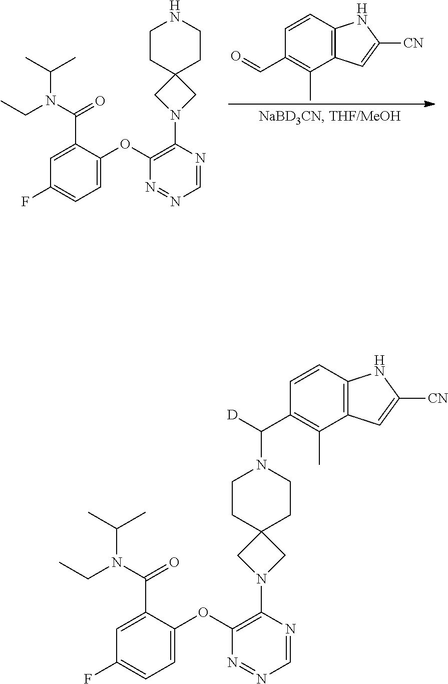

To a reaction flask were added successively intermediate 7-1 (100 mg, 0.23 mmol), 5-formyl-4-methyl-1H-indole-2-carbonitrile (86 mg, 0.46 mmol), NaBD₃CN (30 mg, 0.46 mmol), THE (5 ml) and MeOH (5 ml), and the mixture was reacted at room temperature for 3 days. The reaction solution was quenched with 0.2 ml of saturated ammonium chloride solution and concentrated to obtain the crude product, which was purified with flash column chromatography (eluted with a gradient of water/methanol=100:0-0:100) to obtain 24.3 mg of white solid. MS (m/z): 598.3 [M+H]⁺

¹H NMR (400 MHz, CD₃OD) δ 8.36 (s, 1H), 7.42-7.38 (m, 1H), 7.30-7.23 (m, 3H), 7.22-7.16 (m, 2H), 4.36-4.32 (m, 2H), 3.92-3.88 (m, 2H), 3.84-3.72 (m, 1H), 3.54 (s, 1H), 3.50-3.37 (m, 1H), 3.27-3.14 (m, 1H), 2.54-2.26 (m, 4H), 2.53 (s, 3H), 1.82 (s, 4H), 1.21-0.99 (m, 7H), 0.80-0.75 (m, 2H).

The following compounds were prepared according to the preparation procedure of compound 175 using corresponding intermediates and reagents under appropriate conditions that will be recognized by those skilled in the art.

| | | MS | | Intermediate |
|---|---|---|---|---|
| Compound | Structural formula | (M + H)⁺ | ¹HNMR | used |
| 176 | 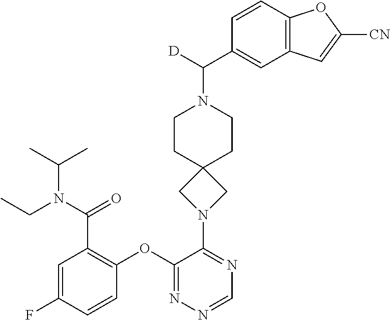 [SMILES: [2H]C(c1ccc2oc(C#N)cc2c1)N1CCC2(CC1)CN(c1ncnnc1Oc1ccc(F)cc1C(=O)N(CC)C(C)C)C2] | 585.3 | ¹H NMR (400 MHz, CD₃OD) δ 8.40 (s, 1H), 8.30 (s, 1H), 7.85 (s, 1H), 7.80 (d, J = 0.7 Hz, 1H), 7.69 (d, J = 8.6 Hz, 1H), 7.64 (dd, J = 8.7, 1.6 Hz, 1H), 7.43-7.36 (m, 1H), 7.31-7.19 (m, 2H), 4.49-4.36 (m, 2H), 4.10-4.06 (m, 1H), 4.03-3.92 (m, 2H), 3.84-3.74 (m, 1H), 3.55-3.40 (m, 1H), 3.27-3.15 (m, 1H), 3.12-2.76 (m, 4H), 2.03-2.00 (m, 3H), 1.31-1.27 (m, 1H), 1.20-1.10 (m, 6H), 1.06 (t, J = 7.1 Hz, 1H), 0.78 (d, J = 5.7 Hz, 2H). | 7-1 |

### Compound 177: 2-((5-(7-((1-(5,6-dimethoxypyridazin-3-yl)piperidin-4-yl)methyl)-2,7-diazaspiro[3.5]nonan-2-yl)-1,2,4-triazin-6-yl)oxy)-5-fluoro-N-isopropylbenzamide

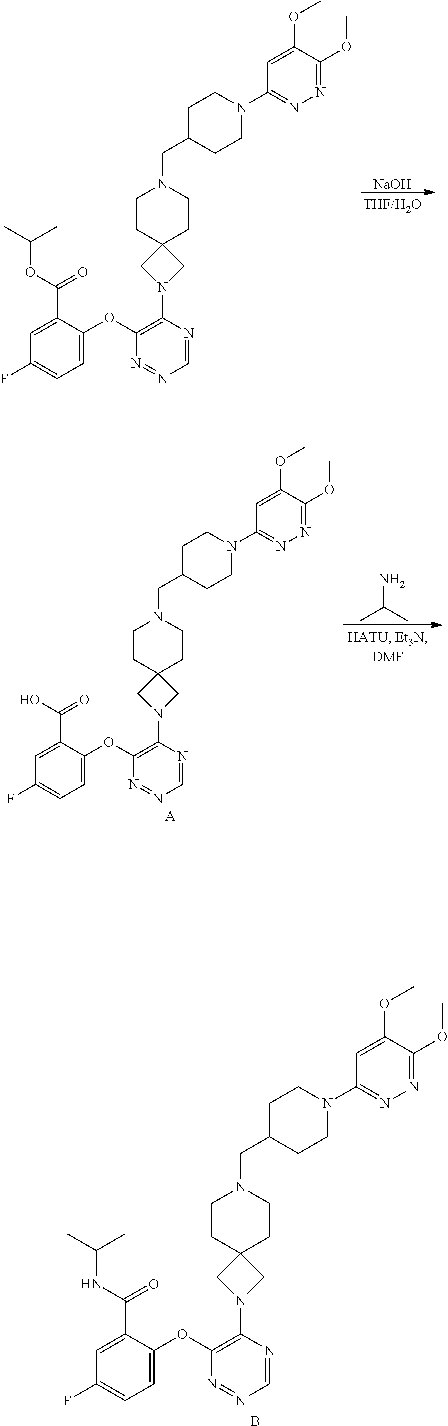

### (A) 2-((5-(7-((1-(5,6-dimethoxypyridazin-3-yl)piperidin-4-yl)methyl)-2,7-diazaspiro[3.5]nonan-2-yl)-1,2,4-triazin-6-yl)oxy)-5-fluorobenzoic acid

To a reaction flask were added successively compound 106 (300 mg, 0.47 mmol), sodium hydroxide (57 mg, 1.41 mmol), THF (8 ml) and water (2 ml), and the mixture was reacted at 50° C. for 15 hours. The reaction solution was adjusted to pH 5-6 with 1N dilute hydrochloric acid, concentrated, and then purified with flash column chromatography (eluted with a gradient of water/methanol=100:0-0:100) to obtain 30 mg of white solid. MS (m/z): 595.2 [M+H]⁺

### (B) 2-((5-(7-((1-(5,6-dimethoxypyridazin-3-yl)piperidin-4-yl)methyl)-2,7-diazaspiro[3.5]nonan-2-yl)-1,2,4-triazin-6-yl)oxy)-5-fluoro-N-isopropylbenzamide

To a reaction flask were added successively 2-((5-(7-((1-(5,6-dimethoxypyridazin-3-yl)piperidin-4-yl)methyl)-2,7-diazaspiro[3.5]nonan-2-yl)-1,2,4-triazin-6-yl)oxy)-5-fluorobenzoic acid (30 mg, 0.05 mmol), isopropylamine (6 mg, 0.10 mmol), HATU (23 mg, 0.06 mmol), triethylamine (15 mg, 0.15 mmol) and DMF (2 ml), and the mixture was reacted at room temperature for 2 hours. The reaction solution was purified with flash column chromatography (eluted with a gradient of water/methanol=100:0-0:100) to obtain 7 mg of light yellow solid. MS (m/z): 636.6 [M+H]+

¹H NMR (400 MHz, CD₃OD) δ 8.33 (d, J=0.7 Hz, 1H), 7.35-7.25 (m, 3H), 6.67 (s, 1H), 4.58 (s, 1H), 4.43 (s, 2H), 4.12 (d, J=13.0 Hz, 2H), 3.94 (s, 3H), 3.87 (s, 3H), 2.85 (t, J=11.7 Hz, 2H), 2.43 (s, 3H), 2.22 (d, J=6.6 Hz, 2H), 1.93-1.82 (m, 7H), 1.25 (d, J=16.5 Hz, 5H), 1.08 (s, 3H), 1.06 (s, 3H).

### Compound 178: 2-((5-(7-((1-(5,6-dimethoxypyridazin-3-yl)piperidin-4-yl)methyl)-2,7-diazaspiro[3.5]nonan-2-yl)-1,2,4-triazin-6-yl)oxy)-N-ethyl-5-fluorobenzamide and Compound 179: (Z)-2-((5-(7-((1-(5,6-dimethoxypyridazin-3-yl)piperidin-4-yl)methyl)-2,7-diazaspiro[3.5]nonan-2-yl)-1,2,4-triazin-6-yl)oxy)-N-ethyl-5-fluorobenzimidic acid

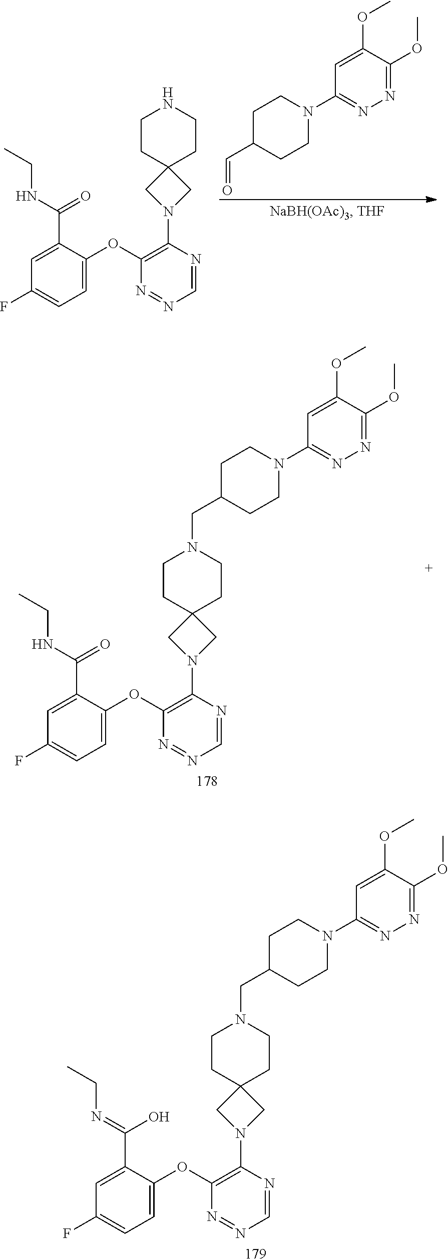

To a reaction flask were added successively intermediate 7-26 (72 mg, 0.19 mmol), intermediate 24-3 (81 mg, 0.32 mmol), NaBH(OAc)₃ (81 mg, 0.38 mmol) and THF (5 ml), and the mixture was reacted at room temperature for 5 hours. The reaction solution was quenched with 0.5 ml of saturated ammonium chloride, concentrated, and then purified with flash column chromatography (eluted with a gradient of water/methanol=100:0-0:100) to obtain two light yellow solid products, which were compound 178 (26.6 mg) and compound 179 (14.2 mg).

### Compound 178: MS (m/z): 622.4 [M+H]⁺

¹H NMR (400 MHz, CD₃OD) δ 8.86 (s, 1H), 7.50-7.37 (m, 3H), 7.05 (s, 1H), 4.73-4.70 (m, 1H), 4.45-4.42 (m, 1H), 4.35-4.32 (m, 1H), 4.21-4.17 (m, 2H), 4.08-4.05 (m, 4H), 3.99-3.94 (m, 4H), 3.69-3.65 (m, 2H), 3.35-3.32 (m, 1H), 3.29-3.25 (m, 1H), 3.16-3.06 (m, 4H), 2.39-2.32 (m, 5H), 2.11-2.06 (m, 2H), 1.51-1.46 (m, 2H), 1.29-1.26 (m, 1H), 1.16-1.11 (m, 3H).

Compound 179: MS (m/z): 622.4 [M+H]⁺

¹H NMR (400 MHz, CD₃OD) δ 8.51 (s, 1H), 8.28 (s, 1H), 7.05-7.02 (m, 1H), 6.95-6.92 (m, 1H), 6.83 (s, 1H), 6.63-6.60 (m, 1H), 4.42-4.32 (m, 1H), 4.19-4.14 (m, 3H), 4.04-4.00 (m, 3H), 3.95 (s, 6H), 3.28-3.15 (m, 4H), 3.09-3.05 (m, 2H), 2.99-2.96 (m, 2H), 2.22-2.17 (m, 5H), 1.98-1.94 (m, 2H), 1.43-1.38 (m, 2H), 1.26-1.20 (m, 3H).

### Compound 184: 2-((5-(7-((1-(5,6-dimethoxypyridazin-3-yl)piperidin-4-yl)methyl)-2,7-diazaspiro[3.5]nonan-2-yl)-1,2,4-triazin-6-yl)oxy)-5-fluorobenzoic acid

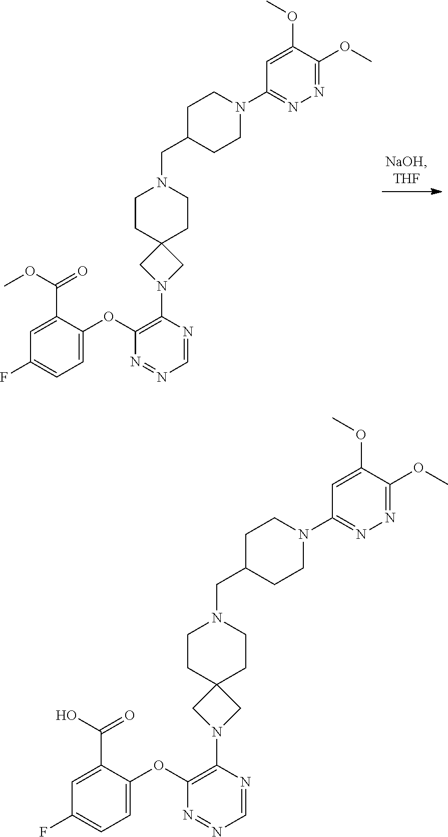 [SMILES: COC(=O)c1cc(F)ccc1Oc1nncnc1N1CC2(CCN(CC3CCN(c4cc(OC)c(OC)nn4)CC3)CC2)C1 ; COc1cc(N2CCC(CN3CCC4(CC3)CN(c3ncnnc3Oc3ccc(F)cc3C(=O)O)C4)CC2)nnc1OC]

To a reaction flask were added successively methyl 2-((5-(7-((1-(5,6-dimethoxypyridazin-3-yl)piperidin-4-yl)methyl)-2,7-diazaspiro[3.5]nonan-2-yl)-1,2,4-triazin-6-yl)oxy)-5-fluorobenzoate (250 mg, 0.41 mmol; prepared from intermediate 7-24 and intermediate 24-3 according to the preparation procedure of compound 57), NaOH (33 mg, 0.82 mmol), THF (4 ml) and water (1 ml), and the mixture was stirred at room temperature for 16 hours. After the reaction was completed, the reaction solution was adjusted to neutral with dilute hydrochloric acid, concentrated, and the residue was purified with flash column chromatography (eluted with a gradient of water/methanol=100:0-0:100) to obtain 4 mg of white solid. MS (m/z): 595.3 [M+H]⁺

¹H NMR (400 MHz, CD₃OD) δ 8.41 (s, 1H), 8.22 (s, 1H), 7.39 (d, J=8.7 Hz, 1H), 7.14 (dd, J=8.7, 4.7 Hz, 1H), 7.06 (t, J=6.7 Hz, 1H), 6.63 (s, 1H), 4.49 (s, 2H), 4.07 (d, J=12.7 Hz, 2H), 3.93 (s, 2H), 3.86 (d, J=0.6 Hz, 3H), 3.80 (s, 3H), 3.03 (s, 3H), 2.83 (t, J=11.2 Hz, 2H), 2.77 (s, 2H), 2.12-1.92 (m, 8H), 1.50 (s, 2H).

### Example 3: Determination of the Activity of the Compounds of the Present Invention on Menin and MLL Protein Binding at a Molecular Level (Fluorescence Polarization Method)

### 1. Reagents, Materials and Instruments:

Recombinant human Menin protein (2-610): ChemPartner, CP2021020801;
MLL polypeptide (6-FAM-Ahx-SG-40): GL Biochem, 865700;
Trizma® hydrochloride buffer solution (Tris): Sigma, T2194-1 L;
NaCl: Invitrogen, AM9760G;
TCEP: Sigma, 646547;
DMSO: Sigma, D4540;
384-well plate: Corning, 4514;
Envision: PerkinElmer.

### 2. Preparation of Reaction Solutions

1) Buffer solution: 20 mL of Tris and 4 mL of NaCl were added respectively to 376 mL of ddH₂O, with pH adjusted to 7.5, and TCEP was added before testing with a dilution ratio of 1:500.
2) 5× test compounds: the test compound was subjected to gradient dilution with the buffer solution to 5-fold the reaction concentration, so that the final concentrations of the compound were 1.000, 0.333, 0.111, 0.037, 0.012, 0.004, 0.001 and 0.0005 μM, respectively.
3) 2.5× Menin protein reaction solution: the Menin protein was diluted to 5 nM with the buffer solution.
4) 2.5×MLL polypeptide reaction solution: the MLL polypeptide was diluted to 5 nM with the buffer solution.

### 3. Methods

### 1) Binding Reaction (20 μL System)

4 μL of 5× test compound was added to each reaction well of the 384-well plate, and the corresponding volume of 10% DMSO was added to the control wells. Except the control wells, 8 μL of Menin protein reaction solution was added to each well. The reaction solution was incubated at room temperature for 30 minutes, and then 8 μL of MLL polypeptide reaction solution was added to each well. The final concentrations of DMSO in the test compound wells and control wells were all 2%. The 384-well plate was sealed and incubated at room temperature for 30 minutes in the dark.

Three control groups were set up:
a blank control group only containing a buffer solution;
a negative control group only containing MLL polypeptide reaction solution; and
a positive control group containing Menin protein reaction solution and MLL polypeptide reaction solution.

### 2) Detection

The vertical polarization S value and the horizontal polarization P value (excitation wavelength was 480 nm, and emission wavelength was 535 nm) were measured with Envision.

### 4. Data Analysis

$$Calculation of G factor : G = ( ( 1000 - mP ) \times corrected S ) / ( ( 1000 + mP ) \times corrected P )$$

Corrected S is the S value of the negative control group—the S value of the blank control group

Corrected P is the P value of the negative control group—the P value of the blank control group

$$Fluorescence polarization value mP : mP = [ ( S - P \times G ) / ( S + P \times G ) ] \times 1000$$

$$Inhibition rate \% ( IR ) = [ ( {mP}_{positive control} - {mP}_{test sample} ) \text{} / ( {mP}_{positive control} - {mP}_{negative control} ) ] \times 100 \%$$

### 5. IC₅₀ Calculation:

calculated by using software XL-Fit™ (version 5.3) supplied by ID Business Solutions (Guildford, UK), which is an additional software to Microsoft Excel.

### 6. Test Results

| Compound | IC₅₀ | Compound | IC₅₀ | Compound | IC₅₀ | Compound | IC₅₀ |
|---|---|---|---|---|---|---|---|
| no. | (μM) | no. | (μM) | no. | (μM) | no. | (μM) |
| 1 | 0.0136 | 45 | 0.0039 | 89 | 0.0028 | 134 | 0.0013 |
| 2 | 0.0300 | 46 | 0.0011 | 90 | 0.0021 | 135 | 0.0555 |
| 3 | 0.0024 | 47 | 0.0026 | 91 | 0.0031 | 136 | 0.1839 |
| 4 | 0.0032 | 48 | 0.0047 | 92 | 0.0109 | 137 | 0.1412 |
| 5 | 0.0213 | 49 | 0.0185 | 93 | 0.0006 | 138 | 0.1019 |
| 6 | 0.0067 | 50 | 0.0054 | 94 | 0.0008 | 139 | 0.0061 |
| 7 | 0.0502 | 51 | 0.0065 | 95 | 0.0032 | 140 | 0.0316 |
| 8 | 0.0346 | 52 | 0.0009 | 96 | 0.0300 | 141 | 0.1534 |
| 9 | 0.0376 | 53 | 0.0990 | 97 | 0.0009 | 142 | 0.1242 |
| 10 | 0.0417 | 54 | 0.0158 | 98 | 0.0071 | 143 | >1 |
| 11 | 0.0051 | 55 | 0.0035 | 99 | 0.0147 | 144 | 0.6162 |
| 12 | 0.0198 | 56 | 0.0284 | 100 | 0.0019 | 145 | >1 |
| 13 | 0.0145 | 57 | 0.0030 | 101 | 0.0009 | 146 | >1 |
| 14 | 0.0295 | 58 | 0.1143 | 102 | 0.0159 | 147 | 0.1163 |
| 15 | 0.0418 | 59 | >1 | 103 | 0.1144 | 148 | 0.0053 |
| 16 | 0.0750 | 60 | 0.5744 | 104 | >1 | 149 | 0.0029 |
| 17 | 0.0487 | 61 | 0.0894 | 105 | 0.4830 | 150 | 0.0415 |
| 18 | 0.0146 | 62 | 0.3238 | 106 | 0.0009 | 151 | 0.0015 |
| 19 | 0.3690 | 63 | >1 | 108 | 0.0216 | 152 | 0.3574 |
| 20 | 0.0987 | 64 | 0.1634 | 109 | 0.0074 | 153 | 0.0052 |
| 21 | 0.0979 | 65 | 0.0839 | 110 | 0.0007 | 154 | 0.0008 |
| 22 | 0.1119 | 66 | 0.0012 | 111 | 0.0052 | 155 | 0.2924 |
| 23 | 0.0232 | 67 | 0.5118 | 112 | 0.0059 | 156 | 0.0437 |
| 24 | 0.0230 | 68 | 0.0502 | 113 | 0.0019 | 157 | 0.3052 |
| 25 | 0.1630 | 69 | 0.0085 | 114 | 0.0023 | 158 | 0.2120 |
| 26 | 0.0009 | 70 | >1 | 115 | 0.0047 | 159 | 0.4495 |
| 27 | 0.0053 | 71 | >1 | 116 | 0.0535 | 160 | 0.0877 |
| 28 | 0.0022 | 72 | 0.0280 | 117 | 0.1753 | 161 | 0.0187 |
| 29 | 0.0025 | 73 | >1 | 118 | >1 | 162 | 0.0139 |
| 30 | 0.0017 | 74 | 0.4175 | 119 | 0.0023 | 163 | 0.0049 |
| 31 | 0.1553 | 75 | 0.9546 | 120 | 0.0152 | 164 | 0.0046 |
| 32 | 0.0010 | 76 | 0.8785 | 121 | 0.0015 | 165 | 0.0053 |
| 33 | 0.0029 | 77 | 0.0738 | 122 | >0.2 | 166 | 0.0026 |
| 34 | 0.0081 | 78 | 0.0288 | 123 | 0.1326 | 167 | 0.0149 |
| 35 | 0.0107 | 79 | 0.0577 | 124 | >0.2 | 168 | >0.2 |
| 36 | 0.0046 | 80 | 0.0066 | 125 | >0.2 | 169 | 0.1663 |
| 37 | 0.0137 | 81 | 0.0549 | 126 | 0.0146 | 170 | 0.0344 |
| 38 | 0.0017 | 82 | 0.0026 | 127 | 0.0243 | 171 | 0.0413 |
| 39 | 0.0683 | 83 | 0.2221 | 128 | 0.0051 | 172 | >0.2 |
| 40 | 0.0114 | 84 | 0.0009 | 129 | 0.1780 | 173 | 0.7496 |
| 41 | 0.0031 | 85 | 0.0042 | 130 | 0.0027 | 107 | 0.0033 |
| 42 | 0.0899 | 86 | 0.0011 | 131 | 0.0180 | 175 | 0.0015 |
| 43 | 0.0064 | 87 | 0.0372 | 132 | 0.0027 | 176 | 0.0061 |
| 44 | 0.0043 | 88 | 0.0011 | 133 | 0.0020 | 177 | 0.0473 |
| 178 | 0.1243 | 180 | 0.0039 | 182 | >1 | 184 | >0.1 |
| 179 | 0.5596 | 181 | 0.0255 | 183 | 0.0040 | | |

### Example 4 Cell Proliferation Assay

### 1. Cell Line

MV-4-11 (ATCC, CRL-9591), a human acute myelogenous leukemia cell line carrying MLL-AF4 mutation. Cells were cultured in an IMDM medium containing 10% heat-inactivated fetal bovine serum (HIFBS) and supplemented with 50 μM β-mercaptoethanol.

### 2. Reagents, Materials and Instruments:

IMDM culture solution: GIBCO, 12440-053;
HIFBS: GIBCO, 10100-147;
β-mercaptoethanol: Sigma, M3148;
CellTiter-Glo® 2.0 assay kit (CTG): Promega, G9243;
DMSO: Sigma, D4540;
96-well cell culture plate: Corning, 3903;
Envision: PerkinElmer.

### 3. Methods

MV-4-11 cells were resuspended in a medium, added to a 96-well plate at a density of 3000 cells/well with a volume of 100 μL/well, and cultured in a cell incubator at 5% CO₂ and 37° C. for 4 hours. The test compound subjected to gradient dilution with the medium was added to a cell culture plate, and the final concentrations of the compound were 1.100, 0.367, 0.122, 0.041, 0.014, 0.005, 0.002 and 0.001 μM, respectively. The corresponding volume of DMSO diluent was added to a blank control well, and the final concentrations of DMSO in the experimental group and control group were both 0.1%. The cell plate was placed in a cell incubator at 5% CO₂ and 37° C. and cultured for 3 days.

### 4. Detection

On day 0 and day 3 of the treatment, 50 μL/well CTG reagent was added to the cell plate and shaken at room temperature in the dark for 10 min, and then the chemiluminescence value of each well was measured with Envision.

### 5. Data Analysis

$$Inhibition rate \% = 100 - ( luminescence {value}_{test sample} - luminescence {value}_{day 0} ) \text{} / ( luminescence {value}_{cell well} - luminescence {value}_{day 0} ) \times 100$$

wherein:
luminescence value_{test sample}: the chemiluminescence value of a cell well treated with the test compound for 3 days.
luminescence value_{day 0}: the chemiluminescence value of a cell well at day 0.
luminescence value_{cell well}: the chemiluminescence value of a cell well not treated with the compound for 3 days.

### 6. GI₅₀ Calculation:

calculated by using software XL-Fit™ (version 5.3) supplied by ID Business Solutions (Guildford, UK), which is an additional software to Microsoft Excel.

### 7. Test Results

| Compound | GI₅₀ | Compound | GI₅₀ | Compound | GI₅₀ | Compound | GI₅₀ |
|---|---|---|---|---|---|---|---|
| no. | (μM) | no. | (μM) | no. | (μM) | no. | (μM) |
| 1 | 0.043 | 45 | 0.007 | 90 | 0.002 | 136 | 0.17 |
| 2 | 0.039 | 46 | 0.002 | 91 | 0.013 | 137 | 0.181 |
| 3 | 0.031 | 47 | 0.003 | 92 | 0.022 | 138 | 0.409 |
| 4 | 0.008 | 48 | 0.015 | 93 | 0.002 | 139 | 0.023 |
| 5 | 0.058 | 49 | 0.024 | 94 | 0.002 | 140 | 0.037 |
| 6 | 0.013 | 50 | 0.007 | 96 | 0.059 | 141 | 0.442 |
| 7 | 0.08 | 51 | 0.009 | 97 | 0.001 | 142 | 0.257 |
| 8 | 0.086 | 52 | 0.003 | 98 | 0.012 | 143 | 2.948 |
| 9 | 0.099 | 53 | 0.087 | 99 | 0.026 | 144 | 0.644 |
| 10 | 0.187 | 54 | 0.021 | 100 | 0.009 | 145 | >3.3 |
| 11 | 0.02 | 55 | 0.004 | 101 | 0.002 | 146 | 0.986 |
| 12 | 0.036 | 56 | 0.045 | 102 | 0.028 | 147 | 1.389 |
| 13 | 0.02 | 57 | 0.005 | 103 | 0.107 | 148 | 0.221 |
| 14 | 0.027 | 58 | 0.23 | 104 | >3.3 | 149 | 0.006 |
| 15 | 0.044 | 59 | >3.3 | 106 | 0.007 | 150 | 0.066 |
| 16 | 0.113 | 60 | 0.693 | 107 | 0.009 | 151 | 0.003 |
| 17 | 0.05 | 61 | 0.11 | 108 | 0.06 | 152 | 0.369 |
| 18 | 0.012 | 62 | 0.193 | 109 | 0.006 | 153 | 0.018 |
| 19 | 0.667 | 64 | 0.226 | 110 | 0.003 | 154 | 0.002 |
| 20 | 0.058 | 66 | 0.003 | 111 | 0.008 | 155 | 0.276 |
| 21 | 0.116 | 67 | 0.309 | 112 | 0.005 | 156 | 0.048 |
| 22 | 0.087 | 68 | 0.071 | 113 | 0.004 | 157 | 0.252 |
| 23 | 0.056 | 69 | 0.016 | 114 | 0.003 | 158 | 0.131 |
| 24 | 0.054 | 70 | 2.147 | 115 | 0.006 | 159 | 0.642 |
| 25 | 0.231 | 71 | 1.284 | 116 | 0.07 | 160 | 0.054 |
| 26 | 0.007 | 72 | 0.081 | 117 | 0.255 | 161 | 0.022 |
| 27 | 0.013 | 73 | 1.27 | 118 | >3.3 | 162 | 0.03 |
| 28 | 0.009 | 74 | 0.176 | 119 | 0.003 | 163 | 0.049 |
| 29 | 0.015 | 75 | 0.883 | 120 | 0.036 | 164 | 0.027 |
| 30 | 0.005 | 76 | 0.932 | 121 | 0.003 | 165 | 0.013 |
| 31 | 0.284 | 77 | 0.06 | 122 | 0.119 | 166 | 0.007 |
| 32 | 0.004 | 78 | 0.02 | 123 | 0.039 | 167 | 0.018 |
| 33 | 0.018 | 79 | 0.078 | 124 | 0.505 | 168 | 0.133 |
| 34 | 0.024 | 80 | 0.016 | 125 | 0.585 | 169 | 0.108 |
| 35 | 0.035 | 81 | 0.117 | 126 | 0.027 | 170 | 0.049 |
| 36 | 0.016 | 82 | 0.007 | 127 | 0.242 | 171 | 0.056 |
| 37 | 0.023 | 84 | 0.002 | 128 | 0.07 | 172 | 0.076 |
| 38 | 0.002 | 85 | 0.006 | 129 | 0.199 | 173 | 0.292 |
| 39 | 0.097 | 86 | 0.006 | 130 | 0.009 | 175 | 0.004 |
| 40 | 0.023 | 87 | 0.156 | 131 | 0.018 | 176 | 0.026 |
| 41 | 0.008 | 88 | 0.004 | 132 | 0.009 | 177 | 0.092 |
| 42 | 0.037 | 89 | 0.004 | 133 | 0.004 | 178 | 0.126 |
| 43 | 0.013 | 95 | 0.009 | 134 | 0.002 | 179 | 0.155 |
| 44 | 0.014 | | | 135 | 0.022 | 180 | 0.010 |
| 181 | 0.007 | 182 | 0.430 | 183 | 0.002 | 184 | >1.1 |

1. A compound of formula (I): 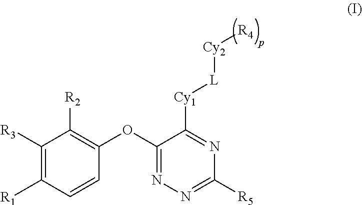
or a pharmaceutically acceptable salt, a solvate, a racemic mixture, an enantiomer, a diastereomer or a tautomer thereof, or a deuterate thereof, wherein
R₁ is selected from hydrogen, halogen, —CN, —OH, —NH₂, C₁₋₆ alkyl, C₁₋₆ haloalkyl, —O—(C₁₋₆ alkyl) and —O—(C₁₋₆ haloalkyl);
R₂ is selected from hydrogen, halogen, —CN, —OH, —NH₂, C₁₋₆ alkyl, C₁₋₆ haloalkyl, —(C₁₋₆ alkyl)-O—(C₁₋₆ alkyl), —(C₁₋₆ alkyl)-OH, —(C₁₋₆ alkyl)-CN, —O—(C₁₋₆ alkyl), —O—(C₁₋₆ haloalkyl), —O—(C₃₋₈ cycloalkyl), —O-(4-8 membered heterocyclyl), C₃₋₈ cycloalkyl, 4-8 membered heterocyclyl, phenyl, 5-12 membered heteroaryl, —S—(C₁₋₆ alkyl), —S—(C₃₋₈ cycloalkyl), —S-(4-8 membered heterocyclyl), —NH(C₁₋₆ alkyl), —N(C₁₋₆ alkyl)₂, —NHCONH₂, —NHCO(C₁₋₆ alkyl), —CONRₐR_{b}, —CSNRₐR_{b}, —COR_{c} and —COORₑ, wherein the C₃₋₈ cycloalkyl, 4-8 membered heterocyclyl, phenyl and 5-12 membered heteroaryl are each optionally substituted with one or more groups independently selected from halogen, —OH, oxo, —CN, —NH₂, —CONH₂, C₁₋₆ alkyl, C₂₋₆ alkenyl, C₂₋₆ alkynyl, C₁₋₆ haloalkyl, —(C₁₋₆ alkyl)-O—(C₁₋₆ alkyl), —(C₁₋₆ alkyl)-OH, —(C₁₋₆ alkyl)-CN, —O—(C₁₋₆ alkyl), —O—(C₁₋₆ haloalkyl), —S—(C₁₋₆ alkyl), —NH(C₁₋₆ alkyl), —N(C₁₋₆ alkyl)₂, —CONH(C₁₋₆ alkyl) and —CON(C₁₋₆ alkyl)₂;
R₃ is selected from hydrogen, halogen, —CN, —OH, —NH₂, C₁₋₆ alkyl, C₁₋₆ haloalkyl, —O—(C₁₋₆ alkyl) and —O—(C₁₋₆ haloalkyl);
or R₂ and R₃ together with the carbon atom to which they are attached form 5-6 membered heteroaryl or 4-6 membered heterocyclyl, wherein the 5-6 membered heteroaryl and 4-6 membered heterocyclyl are each optionally substituted with one or more groups independently selected from halogen, —OH, oxo, —CN, —NH₂, —CONH₂, C₁₋₆ alkyl, C₂₋₆ alkenyl, C₂₋₆ alkynyl, C₁₋₆ haloalkyl, —(C₁₋₆ alkyl)-O—(C₁₋₆ alkyl), —(C₁₋₆ alkyl)-OH, —(C₁₋₆ alkyl)-CN, —O—(C₁₋₆ alkyl) and —O—(C₁₋₆ haloalkyl);
R₅ is selected from hydrogen, halogen, —CN, —OH, C₁₋₆ alkyl, C₁₋₆ haloalkyl, —(C₁₋₆ alkyl)-O—(C₁₋₆ alkyl), —(C₁₋₆ alkyl)-OH, —(C₁₋₆ alkyl)-CN, —O—(C₁₋₆ alkyl), —O—(C₁₋₆ haloalkyl), C₃₋₈ cycloalkyl, 4-8 membered heterocyclyl, —S—(C₁₋₆ alkyl), —NHCONH₂, —NHCO(C₁₋₆ alkyl) and —NRₐR_{b};
Cy₁ is 4-12 membered heterocyclyl, which is optionally substituted with one or more groups independently selected from halogen, —OH, oxo, —CN, —NH₂, —CONH₂, C₁₋₆ alkyl, C₂₋₆ alkenyl, C₂₋₆ alkynyl, C₁₋₆ haloalkyl, —(C₁₋₆ alkyl)-O—(C₁₋₆ alkyl), —(C₁₋₆ alkyl)-OH, —(C₁₋₆ alkyl)-CN, —O—(C₁₋₆ alkyl) and —O—(C₁₋₆ haloalkyl);
Cy₂ is selected from C₃₋₈ cycloalkyl, 4-9 membered heterocyclyl, aryl and 5-14 membered heteroaryl;
R₄ is independently selected from halogen, —CN, —OH, oxo, —SH, C₁₋₆ alkyl, C₂₋₆ alkenyl, C₂₋₆ alkynyl, —O—(C₁₋₆ alkyl), —O—(C₁₋₆ haloalkyl), —O—(C₃₋₈ cycloalkyl), —O-(4-8 membered heterocyclyl), —(C₁₋₆ alkyl)ₘ-(C₃₋₈ cycloalkyl), —(C₁₋₆ alkyl)ₘ-(4-8 membered heterocyclyl), —(C₁₋₆ alkyl)ₘ-phenyl, —(C₁₋₆ alkyl)ₘ-(5-12 membered heteroaryl), —S—(C₁₋₆ alkyl), —S—(C₃₋₈ cycloalkyl), —S-(4-8 membered heterocyclyl), —NHCONH₂, —CONRₐR_{b}, —COR_{c}, —COOR_{c}, —NRₐR_{b}, —NR_{d}COR_{c}, —NR_{d}S(O)ₙR_{f}, —S(O)ₙR_{f} and —S(O)ₙNRₐR_{b}, wherein the C₁₋₆ alkyl, C₃₋₈ cycloalkyl, 4-8 membered heterocyclyl, phenyl and 5-12 membered heteroaryl are each optionally substituted with one or more groups independently selected from halogen, —OH, oxo, —SH, —CN, —NH₂, —CONH₂, C₁₋₆ alkyl, C₂₋₆ alkenyl, C₂₋₆ alkynyl, C₁₋₆ haloalkyl, —(C₁₋₆ alkyl)-O—(C₁₋₆ alkyl), —(C₁₋₆ alkyl)-OH, —(C₁₋₆ alkyl)-CN, —O—(C₁₋₆ alkyl), —O—(C₁₋₆ haloalkyl), —O—(C₃₋₈ cycloalkyl), —O-(4-8 membered heterocyclyl), —S—(C₁₋₆ alkyl), —NH(C₁₋₆ alkyl), —N(C₁₋₆ alkyl)₂, —CO(C₁₋₆ alkyl), —CO(C₂₋₆ alkenyl), —CO(C₂₋₆ alkynyl), —CONH(C₁₋₆ alkyl), —CON(C₁₋₆ alkyl)₂, C₃₋₈ cycloalkyl and 4-8 membered heterocyclyl;
L is absent, or L is CH₂;
Rₐ, R_{b}, R_{c}, R_{d} and Rₑ are each independently selected from hydrogen, C₁₋₆ alkyl, C₂₋₆ alkenyl, C₂₋₆ alkynyl, —(C₁₋₆ alkyl)ₘ-(C₃₋₈ cycloalkyl), —(C₁₋₆ alkyl)ₘ-(4-8 membered heterocyclyl), —(C₁₋₆ alkyl)ₘ-phenyl and —(C₁₋₆ alkyl)ₘ-(5-12 membered heteroaryl), wherein the C₁₋₆ alkyl, C₃₋₈ cycloalkyl, 4-8 membered heterocyclyl, phenyl and 5-12 membered heteroaryl are each optionally substituted with one or more groups independently selected from halogen, —OH, oxo, —SH, —CN, —NH₂, —CONH₂, C₁₋₆ alkyl, C₂₋₆ alkenyl, C₂₋₆ alkynyl, C₁₋₆ haloalkyl, —(C₁₋₆ alkyl)-O—(C₁₋₆ alkyl), —(C₁₋₆ alkyl)-OH, —(C₁₋₆ alkyl)-CN, —O—(C₁₋₆ alkyl), —O—(C₁₋₆ haloalkyl), —O—(C₃₋₈ cycloalkyl), —O-(4-8 membered heterocyclyl), —S—(C₁₋₆ alkyl), —NH(C₁₋₆ alkyl), —N(C₁₋₆ alkyl)₂, —NH(C₃₋₈ cycloalkyl), —NH(4-8 membered heterocyclyl), —CO(C₁₋₆ alkyl), —CO(C₂₋₆ alkenyl), —CO(C₂₋₆ alkynyl), —CO(C₃₋₈ cycloalkyl), —CO(4-8 membered heterocyclyl), —CONH(C₁₋₆ alkyl), —CONH(C₃₋₈ cycloalkyl), —CONH(4-8 membered heterocyclyl) and —CON(C₁₋₆ alkyl)₂;
R_{f} is —CH₃;
p is 0, 1, 2, 3, 4 or 5;
m is 0 or 1; and
n is 1 or 2;
provided that, R₄ is not —NH(6-membered nitrogen-containing heteroaryl) or —NH(phenyl substituted with F).

2. The compound or the pharmaceutically acceptable salt, the solvate, the racemic mixture, the enantiomer, the diastereomer or the tautomer thereof, or the deuterate thereof according to claim 1, wherein
R₁ is halogen, CN or C₁₋₆ haloalkyl;
R₂ is selected from hydrogen, —O—(C₁₋₆ alkyl), C₃₋₈ cycloalkyl, 4-8 membered heterocyclyl, 5-12 membered heteroaryl, —CONRₐR_{b}, —CSNRₐR_{b}, —COR_{c} and —COORₑ, wherein the C₃₋₈ cycloalkyl, 4-8 membered heterocyclyl and 5-12 membered heteroaryl are each optionally substituted with one or more groups independently selected from halogen, —OH, oxo, —CN, —NH₂, —CONH₂, C₁₋₆ alkyl, C₁₋₆ haloalkyl, —(C₁₋₆ alkyl)-O—(C₁₋₆ alkyl), —(C₁₋₆ alkyl)-OH, —(C₁₋₆ alkyl)-CN, —O—(C₁₋₆ alkyl) and —O—(C₁₋₆ haloalkyl);
R₃ is hydrogen;
or R₂ and R₃ together with the carbon atom to which they are attached form 5-6 membered heteroaryl, wherein the 5-6 membered heteroaryl is optionally substituted with one or more groups independently selected from C₁₋₆ alkyl;
R₅ is selected from hydrogen, C₁₋₆ alkyl and —NRₐR_{b};
Cy₁ is 4-12 membered heterocyclyl;
Cy₂ is selected from C₃₋ₛ cycloalkyl, 4-9 membered heterocyclyl, aryl and 5-14 membered heteroaryl;
R₄ is independently selected from halogen, —CN, —OH, oxo, C₁₋₆ alkyl, —O—(C₁₋₆ alkyl), —(C₁₋₆ alkyl)ₘ-(C₃₋₈ cycloalkyl), —(C₁₋₆ alkyl)ₘ-(4-8 membered heterocyclyl), —(C₁₋₆ alkyl)ₘ-phenyl, —(C₁₋₆ alkyl)ₘ-(5-12 membered heteroaryl), —CONRₐR_{b}, —CORₑ, —COORₑ, —NRₐR_{b}, —NR_{d}COR_{c}, —NR_{d}S(O)ₙR_{f}, —S(O)ₙR_{f} and —S(O)ₙNRₐR_{b}, wherein the C₁₋₆ alkyl, C₃₋₈ cycloalkyl, 4-8 membered heterocyclyl, phenyl and 5-12 membered heteroaryl are each optionally substituted with one or more groups independently selected from halogen, —OH, oxo, —CN, —NH₂, —CONH₂, C₁₋₆ alkyl, C₂₋₆ alkenyl, C₂₋₆ alkynyl, C₁₋₆ haloalkyl, —(C₁₋₆ alkyl)-O—(C₁₋₆ alkyl), —(C₁₋₆ alkyl)-OH, —(C₁₋₆ alkyl)-CN, —O—(C₁₋₆ alkyl), —O—(C₁₋₆ haloalkyl), —NH(C₁₋₆ alkyl), —N(C₁₋₆ alkyl)₂, —CONH(C₁₋₆ alkyl), —CON(C₁₋₆ alkyl)₂, C₃₋₈ cycloalkyl and 4-8 membered heterocyclyl;
L is absent, or L is CH₂;
Rₐ, R_{b}, R_{c}, R_{d} and Rₑ are each independently selected from hydrogen, C₁₋₆ alkyl, C₂₋₆ alkenyl, —(C₁₋₆ alkyl)ₘ-(C₃₋₈ cycloalkyl), —(C₁₋₆ alkyl)ₘ-(4-8 membered heterocyclyl), —(C₁₋₆ alkyl)ₘ-phenyl and —(C₁₋₆ alkyl)ₘ-(5-12 membered heteroaryl), wherein the C₁₋₆ alkyl, C₃₋₈ cycloalkyl, 4-8 membered heterocyclyl, phenyl and 5-12 membered heteroaryl are each optionally substituted with one or more groups independently selected from halogen, —OH, —CN, —CONH₂, C₁₋₆ alkyl, C₁₋₆ haloalkyl, —(C₁₋₆ alkyl)-OH, —O—(C₁₋₆ alkyl), —NH(C₃₋₈ cycloalkyl), —CO(C₂₋₆ alkenyl) and —CON(C₁₋₆ alkyl)₂;
R_{f} is —CH₃;
p is 0, 1, 2 or 3;
m is 0 or 1; and
n is 1 or 2;
provided that, R₄ is not —NH(6-membered nitrogen-containing heteroaryl) or —NH(phenyl substituted with F).

3. The compound or the pharmaceutically acceptable salt, the solvate, the racemic mixture, the enantiomer, the diastereomer or the tautomer thereof, or the deuterate thereof according to any one of claims 1-2, wherein the compound is a compound of formula (I-1): 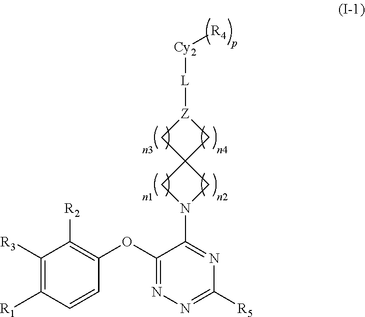
wherein
Z is N or CH; preferably, Z is N; and
n1, n2, n3 and n4 are each independently selected from 1 and 2.

4. The compound or the pharmaceutically acceptable salt, the solvate, the racemic mixture, the enantiomer, the diastereomer or the tautomer thereof, or the deuterate thereof according to any one of claims 1-2, wherein the compound is a compound of formula (I-2): 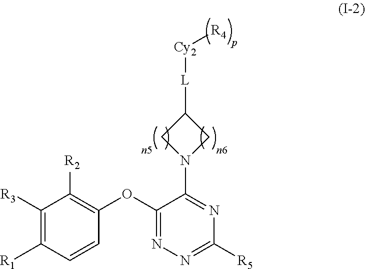
wherein n5 and n6 are each independently selected from 1 and 2.

5. The compound or the pharmaceutically acceptable salt, the solvate, the racemic mixture, the enantiomer, the diastereomer or the tautomer thereof, or the deuterate thereof according to any one of claims 1-4, wherein R₁ is halogen; preferably, R₁ is F or Cl; and more preferably, R₁ is F.

6. The compound or the pharmaceutically acceptable salt, the solvate, the racemic mixture, the enantiomer, the diastereomer or the tautomer thereof, or the deuterate thereof according to any one of claims 1-5, wherein R₂ is selected from hydrogen, —O—(C₁₋₆ alkyl), C₃₋₈ cycloalkyl, 4-8 membered heterocyclyl, 5-12 membered heteroaryl, —CONRₐR_{b}, —CSNRₐR_{b}, —COR_{c} and —COORₑ, wherein Rₐ, R_{b}, R_{c} and Rₑ are each independently selected from hydrogen, C₁₋₆ alkyl, C₁₋₆ haloalkyl, —(C₁₋₆ alkyl)-O—(C₁₋₆ alkyl), —(C₁₋₆ alkyl)-OH, —(C₁₋₆ alkyl)-CN, C₃₋₈ cycloalkyl and 4-8 membered heterocyclyl, wherein the C₃₋₈ cycloalkyl, 4-8 membered heterocyclyl and 5-12 membered heteroaryl are each optionally substituted with one or more groups independently selected from halogen, —OH, oxo, —CN, —NH₂, —CONH₂, C₁₋₆ alkyl, C₁₋₆ haloalkyl, —(C₁₋₆ alkyl)-O—(C₁₋₆ alkyl), —(C₁₋₆ alkyl)-OH, —(C₁₋₆ alkyl)-CN, —O—(C₁₋₆ alkyl) and —O—(C₁₋₆ haloalkyl);
preferably, R₂ is selected from hydrogen, —O—(C₁₋₆ alkyl), C₃₋₆ cycloalkyl, 4-6 membered heterocyclyl, 5-6 membered heteroaryl, —CONRₐR_{b}, —CSNRₐR_{b}, —COR_{c} and —COORₑ, wherein Rₐ, R_{b}, R_{c} and Rₑ are each independently selected from C₁₋₆ alkyl, C₃₋₆ cycloalkyl and 4-6 membered heterocyclyl, wherein the C₃₋₆ cycloalkyl, 4-6 membered heterocyclyl and 5-6 membered heteroaryl are each optionally substituted with one or more groups independently selected from halogen, —OH and C₁₋₆ alkyl;
more preferably, R₂ is selected from —COO(C₁₋₆ alkyl) and —CON(C₁₋₆ alkyl)₂; and
most preferably, R₂ is —CON(C₁₋₆ alkyl)₂.

7. The compound or the pharmaceutically acceptable salt, the solvate, the racemic mixture, the enantiomer, the diastereomer or the tautomer thereof, or the deuterate thereof according to claim 1, wherein R₃ is hydrogen.

8. The compound or the pharmaceutically acceptable salt, the solvate, the racemic mixture, the enantiomer, the diastereomer or the tautomer thereof, or the deuterate thereof according to any one of claims 1-5, wherein R₂ and R₃ together with the carbon atom to which they are attached form 5-6 membered heteroaryl, wherein the 5-6 membered heteroaryl is optionally substituted with one or more groups independently selected from C₁₋₆ alkyl; preferably, R₂ and R₃ together with the carbon atom to which they are attached form pyrazolyl, wherein the pyrazolyl is optionally substituted with one or more groups independently selected from C₁₋₆ alkyl.

9. The compound or the pharmaceutically acceptable salt, the solvate, the racemic mixture, the enantiomer, the diastereomer or the tautomer thereof, or the deuterate thereof according to any one of claims 1-8, wherein R₅ is selected from hydrogen, C₁₋₆ alkyl, —NH₂, —NH(C₁₋₆ alkyl) and —N(C₁₋₆ alkyl)₂; preferably, R₅ is selected from hydrogen, C₁₋₆ alkyl and —NH(C₁₋₆ alkyl); and more preferably, R₅ is hydrogen.

10. The compound or the pharmaceutically acceptable salt, the solvate, the racemic mixture, the enantiomer, the diastereomer or the tautomer thereof, or the deuterate thereof according to any one of claims 1-9, wherein L is CH₂.

11. The compound or the pharmaceutically acceptable salt, the solvate, the racemic mixture, the enantiomer, the diastereomer or the tautomer thereof, or the deuterate thereof according to any one of claims 1-10, wherein Cy₂ is selected from C₃₋₈ cycloalkyl, 4-8 membered heterocyclyl, phenyl and 5-12 membered heteroaryl; preferably, Cy₂ is selected from C₃₋₆ cycloalkyl, 4-6 membered heterocyclyl, phenyl, 5-6 membered heteroaryl and 8-10 membered heteroaryl.

12. The compound or the pharmaceutically acceptable salt, the solvate, the racemic mixture, the enantiomer, the diastereomer or the tautomer thereof, or the deuterate thereof according to claim 11, wherein Cy₂ is selected from cyclopropyl, cyclobutyl, cyclohexyl, cyclohexenyl, pyrrolidyl, piperidyl, piperazinyl, phenyl, pyridyl, pyridinonyl, pyrimidyl, indolyl, indazolyl, benzofuranyl, benzoxazolyl, imidazopyridyl, quinolinyl, quinolinonyl, quinazolinyl and dihydro-[1,4]dioxinopyridyl.

13. The compound or the pharmaceutically acceptable salt, the solvate, the racemic mixture, the enantiomer, the diastereomer or the tautomer thereof, or the deuterate thereof according to claim 12, wherein Cy₂ is selected from 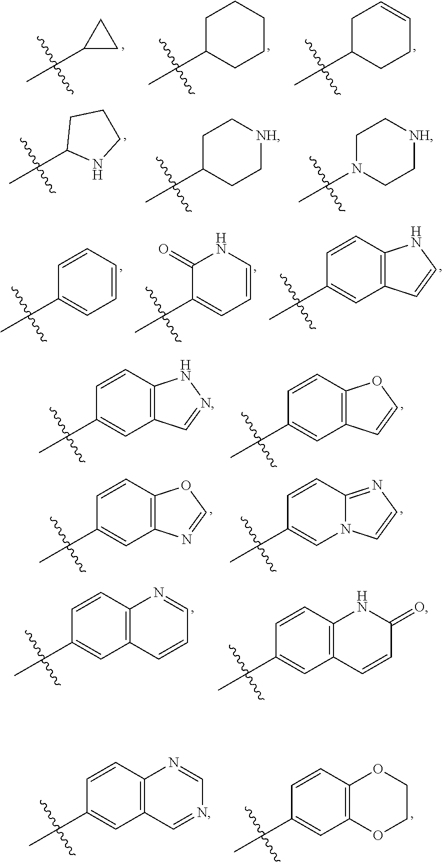
preferably, Cy₂ is selected from 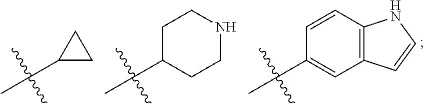
and more preferably, Cy₂ is 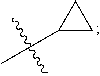 [SMILES: *C1CC1]
or Cy₂ is 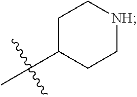 [SMILES: *C1CCNCC1]
or Cy₂ is

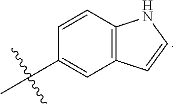 [SMILES: *c1ccc2[nH]ccc2c1]

14. The compound or the pharmaceutically acceptable salt, the solvate, the racemic mixture, the enantiomer, the diastereomer or the tautomer thereof, or the deuterate thereof according to any one of claims 1-13, wherein R₄ is independently selected from halogen, —CN, —OH, oxo, C₁₋₆ alkyl, —O—(C₁₋₆ alkyl), —(C₁₋₆ alkyl)ₘ-(C₃₋₈ cycloalkyl), —(C₁₋₆ alkyl)ₘ-(4-8 membered heterocyclyl), —(C₁₋₆ alkyl)ₘ-phenyl, —(C₁₋₆ alkyl)ₘ-(5-12 membered heteroaryl), —CONRₐR_{b}, —COR_{c}, —COORₑ, —NRₐR_{b}, —NR_{d}COR_{c}, —NR_{d}S(O)ₙR_{f} and —S(O)˜R_{f}, wherein the C₁₋₆ alkyl, C₃₋₈ cycloalkyl, 4-8 membered heterocyclyl, phenyl and 5-12 membered heteroaryl are each optionally substituted with one or more groups independently selected from halogen, —OH, oxo, —CN, —NH₂, —CONH₂, C₁₋₆ alkyl, C₂₋₆ alkenyl, C₂₋₆ alkynyl, C₁₋₆ haloalkyl, —(C₁₋₆ alkyl)-O—(C₁₋₆ alkyl), —(C₁₋₆ alkyl)-OH, —(C₁₋₆ alkyl)-CN, —O—(C₁₋₆ alkyl), —O—(C₁₋₆ haloalkyl), —NH(C₁₋₆ alkyl), —N(C₁₋₆ alkyl)₂, —CONH(C₁₋₆ alkyl), —CON(C₁₋₆ alkyl)₂, C₃₋₈ cycloalkyl and 4-8 membered heterocyclyl;
preferably, R₄ is independently selected from halogen, —CN, —OH, oxo, C₁₋₆ alkyl, —O—(C₁₋₆ alkyl), —(C₁₋₆ alkyl)ₘ-(4-8 membered heterocyclyl), —(C₁₋₆ alkyl)ₘ-phenyl, —(C₁₋₆ alkyl)ₘ-(5-12 membered heteroaryl), —CONRₐR_{b}, —COR_{c}, —COORₑ, —NRₐR_{b}, —NR_{d}COR_{c}, —NR_{d}S(O)ₙR_{f} and —S(O)ₙR_{f}, wherein the C₁₋₆ alkyl, 4-8 membered heterocyclyl, phenyl and 5-12 membered heteroaryl are each optionally substituted with one or more groups independently selected from halogen, —OH, oxo, —CN, —NH₂, —CONH₂, C₁₋₆ alkyl, C₂₋₆ alkenyl, C₂₋₆ alkynyl, C₁₋₆ haloalkyl, —(C₁₋₆ alkyl)-OH, —O—(C₁₋₆ alkyl) and C₃₋₈ cycloalkyl;
provided that, R₄ is not —NH(6-membered nitrogen-containing heteroaryl) or —NH(phenyl substituted with F).

15. The compound or the pharmaceutically acceptable salt, the solvate, the racemic mixture, the enantiomer, the diastereomer or the tautomer thereof, or the deuterate thereof according to claim 14, wherein R₄ is independently selected from:
1) halogen;
2) —CN;
3) —OH;
4) oxo;
5) C₁₋₆ alkyl, which is optionally substituted with one or more groups independently selected from —OH, —CN and —CONH₂;
6) —O—(C₁₋₆ alkyl);
7) —(C₁₋₆ alkyl)ₘ-(4-6 membered heterocyclyl), wherein preferably, the 4-6 membered heterocyclyl is selected from oxetanyl and dihydropyranyl;
8) —(C₁₋₆ alkyl)ₘ-phenyl, wherein the phenyl is optionally substituted with one or more groups independently selected from —CN;
9) —(C₁₋₆ alkyl)ₘ-(5-12 membered heteroaryl), wherein preferably, the 5-12 membered heteroaryl is selected from 5-6 membered heteroaryl and 8-10 membered heteroaryl; more preferably, the 5-12 membered heteroaryl is selected from pyrazolyl, oxazolyl, pyridyl, pyridinonyl, pyrimidyl, pyridazinyl, pyridazinonyl, pyrazinyl, 1,2,4-triazinyl, indolyl, imidazopyridazinyl, [1,2,4]triazolopyridyl, pyrazolopyrimidyl, dihydro-pyrimidopyridazinyl and dihydro-[1,4]dioxinopyridazinyl; and most preferably, the 5-12 membered heteroaryl is selected from pyridyl and pyridazinyl;
wherein the 5-12 membered heteroaryl is optionally substituted with one or more groups independently selected from halogen, —OH, oxo, —CN, —NH₂, —CONH₂, C₁₋₆ alkyl, C₂₋₆ alkenyl, C₂₋₆ alkynyl, C₁₋₆ haloalkyl, —(C₁₋₆ alkyl)-OH, —O—(C₁₋₆ alkyl) and C₃₋₈ cycloalkyl; preferably, the 5-12 membered heteroaryl is optionally substituted with one or more groups independently selected from halogen, —OH, —CN, —CONH₂, C₁₋₆ alkyl, C₂₋₆ alkenyl, C₂₋₆ alkynyl, C₆ haloalkyl, —(C₁₋₆ alkyl)-OH, —O—(C₁₋₆ alkyl) and C₃₋₈ cycloalkyl;
10) —CONRₐR_{b}, wherein Rₐ and R_{b} are each independently selected from hydrogen, C₁₋₆ alkyl, —(C₁₋₆ alkyl)ₘ-(C₃₋₆ cycloalkyl), —(C₁₋₆ alkyl)ₘ-(4-6 membered heterocyclyl), —(C₁₋₆ alkyl)ₘ-phenyl and —(C₁₋₆ alkyl)ₘ-(5-6 membered heteroaryl), wherein the C₁₋₆ alkyl, C₃₋₆ cycloalkyl, 4-6 membered heterocyclyl, phenyl and 5-6 membered heteroaryl are each optionally substituted with one or more groups independently selected from halogen and —O—(C₁₋₆ alkyl);
11) —COR_{c}, wherein R_{c} is selected from C₁₋₆ alkyl, C₂₋₆ alkenyl, —(C₁₋₆ alkyl)ₘ-(C₃₋₈ cycloalkyl), —(C₁₋₆ alkyl)ₘ-(4-6 membered heterocyclyl), —(C₁₋₆ alkyl)ₘ-phenyl and —(C₁₋₆ alkyl)ₘ-(5-10 membered heteroaryl), wherein the C₁₋₆ alkyl, C₃₋₈ cycloalkyl, 4-6 membered heterocyclyl, phenyl and 5-10 membered heteroaryl are each optionally substituted with one or more groups independently selected from halogen, —OH, —CN, C₁₋₆ alkyl, C₁₋₆ haloalkyl, —O—(C₁₋₆ alkyl), —NH(C₃₋₆ cycloalkyl), —CO(C₂₋₆ alkenyl) and —CON(C₁₋₆ alkyl)₂;
12) —COORₑ, wherein Rₑ is selected from hydrogen and C₁₋₆ alkyl;
13) —NRₐR_{b}, wherein Rₐ and R_{b} are each independently selected from hydrogen, C₁₋₆ alkyl and —(C₁₋₆ alkyl)ₘ-(5-10 membered heteroaryl), wherein the C₁₋₆ alkyl and 5-10 membered heteroaryl are each optionally substituted with one or more groups independently selected from halogen, —OH, —CN, —CONH₂, C₁₋₆ alkyl, —(C₁₋₆ alkyl)-OH and —O—(C₁₋₆ alkyl), provided that, R₄ is not —NH(6-membered nitrogen-containing heteroaryl);
14) —NR_{d}COR_{c}, wherein R_{d} is selected from hydrogen and C₁₋₆ alkyl; R_{c} is C₁₋₆ alkyl, which is optionally substituted with one or more groups independently selected from —CN and —O—(C₁₋₆ alkyl);
15) —NR_{d}S(O)ₙR_{f}, wherein R_{d} is hydrogen; R_{f} is —CH₃;
16) —S(O)ₙR_{f}, wherein R_{f} is —CH₃.

16. The compound or the pharmaceutically acceptable salt, the solvate, the racemic mixture, the enantiomer, the diastereomer or the tautomer thereof, or the deuterate thereof according to claim 1, wherein the compound is a compound of formula (I-3): 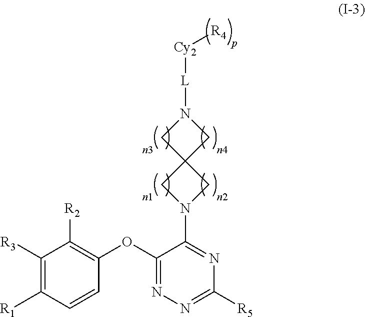
wherein
n1, n2, n3 and n4 are each independently selected from 1 and 2; preferably, both n1 and n2 are 1, and both n3 and n4 are 2;
R₁ is halogen; preferably, R₁ is F or Cl; and more preferably, R₁ is F;
R₂ is selected from hydrogen, —O—(C₁₋₆ alkyl), C₃₋₈ cycloalkyl, 4-8 membered heterocyclyl, 5-12 membered heteroaryl, —CONRₐR_{b}, —CSNRₐR_{b}, —COR_{c} and —COORₑ, wherein Rₐ, R_{b}, R_{c} and Rₑ are each independently selected from hydrogen, C₁₋₆ alkyl, C₁₋₆ haloalkyl, —(C₁₋₆ alkyl)-O—(C₁₋₆ alkyl), —(C₁₋₆ alkyl)-OH, —(C₁₋₆ alkyl)-CN, C₃₋₈ cycloalkyl and 4-8 membered heterocyclyl, wherein the C₃₋₈ cycloalkyl, 4-8 membered heterocyclyl and 5-12 membered heteroaryl are each optionally substituted with one or more groups independently selected from halogen, —OH, oxo, —CN, —NH₂, —CONH₂, C₁₋₆ alkyl, C₁₋₆ haloalkyl, —(C₁₋₆ alkyl)-O—(C₁₋₆ alkyl), —(C₁₋₆ alkyl)-OH, —(C₁₋₆ alkyl)-CN, —O—(C₁₋₆ alkyl) and —O—(C₁₋₆ haloalkyl); preferably, R₂ is selected from hydrogen, —O—(C₁₋₆ alkyl), C₃_6 cycloalkyl, 4-6 membered heterocyclyl, 5-6 membered heteroaryl, —CONRₐR_{b}, —CSNRₐR_{b}, —COR_{c} and —COORₑ, wherein Rₐ, R_{b}, R_{c} and Rₑ are each independently selected from C₁₋₆ alkyl, C₃₋₆ cycloalkyl and 4-6 membered heterocyclyl, wherein the C₃₋₆ cycloalkyl, 4-6 membered heterocyclyl and 5-6 membered heteroaryl are each optionally substituted with one or more groups independently selected from halogen, —OH and C₁₋₆ alkyl; more preferably, R₂ is selected from —COO(C₁₋₆ alkyl) and —CON(C₁₋₆ alkyl)₂; and most preferably, R₂ is —CON(C₁₋₆ alkyl)₂;
R₃ is hydrogen;
or R₂ and R₃ together with the carbon atom to which they are attached form 5-6 membered heteroaryl, wherein the 5-6 membered heteroaryl is optionally substituted with one or more groups independently selected from C₁₋₆ alkyl; preferably, R₂ and R₃ together with the carbon atom to which they are attached form pyrazolyl, wherein the pyrazolyl is optionally substituted with one or more groups independently selected from C₁₋₆ alkyl;
R₅ is selected from hydrogen, C₁₋₆ alkyl, —NH₂, —NH(C₁₋₆ alkyl) and —N(C₁₋₆ alkyl)₂;
preferably, R₅ is selected from hydrogen, C₁_, alkyl and —NH(C₁₋₆ alkyl); and more preferably, R₅ is hydrogen;
Cy₂ is selected from C₃₋₆ cycloalkyl, 4-6 membered heterocyclyl, phenyl, 5-6 membered heteroaryl and 8-10 membered heteroaryl; preferably, Cy₂ is selected from cyclopropyl, cyclobutyl, cyclohexyl, cyclohexenyl, pyrrolidyl, piperidyl, piperazinyl, phenyl, pyridyl, pyridinonyl, pyrimidyl, indolyl, indazolyl, benzofuranyl, benzoxazolyl, imidazopyridyl, quinolinyl, quinolinonyl, quinazolinyl and dihydro-[1,4]dioxinopyridyl; more preferably, Cy₂ is selected from 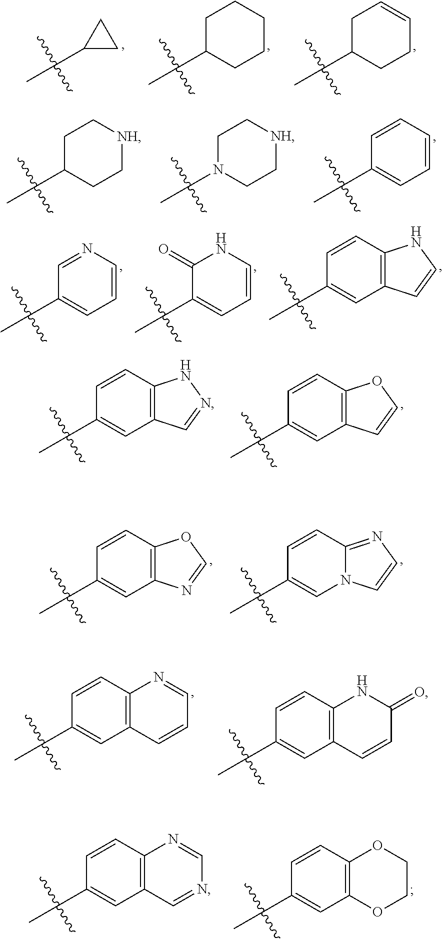
and most preferably, Cy₂ is 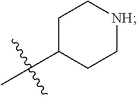 [SMILES: *C1CCNCC1]
R₄ is independently selected from halogen, —CN, —OH, oxo, C₁₋₆ alkyl, —O—(C₁₋₆ alkyl), —(C₁₋₆ alkyl)ₘ-(4-8 membered heterocyclyl), —(C₁₋₆ alkyl)ₘ-phenyl, —(C₁₋₆ alkyl)ₘ-(5-12 membered heteroaryl), —CONRₐR_{b}, —COR_{c}, —COORₑ, —NRₐR_{b}, —NR_{d}COR_{c}, —NR_{d}S(O)ₙR_{f} and —S(O)˜R_{f}, wherein the C₁₋₆ alkyl, 4-8 membered heterocyclyl, phenyl and 5-12 membered heteroaryl are each optionally substituted with one or more groups independently selected from halogen, —OH, oxo, —CN, —NH₂, —CONH₂, C₁₋₆ alkyl, C₂₋₆ alkenyl, C₂₋₆ alkynyl, C₁₋₆ haloalkyl, —(C₁₋₆ alkyl)-OH, —O—(C₁₋₆ alkyl) and C₃₋₈ cycloalkyl; preferably, R₄ is independently selected from:
1) halogen;
2) —CN;
3) —OH;
4) oxo;
5) C₁₋₆ alkyl, which is optionally substituted with one or more groups independently selected from —OH, —CN and —CONH₂;
6) —O—(C₁₋₆ alkyl);
7) —(C₁₋₆ alkyl)ₘ-(4-6 membered heterocyclyl), wherein preferably, the 4-6 membered heterocyclyl is selected from oxetanyl and dihydropyranyl;
8) —(C₁₋₆ alkyl)ₘ-phenyl, wherein the phenyl is optionally substituted with one or more groups independently selected from —CN;
9) —(C₁₋₆ alkyl)ₘ-(5-12 membered heteroaryl), wherein preferably, the 5-12 membered heteroaryl is selected from 5-6 membered heteroaryl and 8-10 membered heteroaryl; more preferably, the 5-12 membered heteroaryl is selected from pyrazolyl, oxazolyl, pyridyl, pyridinonyl, pyrimidyl, pyridazinyl, pyridazinonyl, pyrazinyl, 1,2,4-triazinyl, indolyl, imidazopyridazinyl, [1,2,4]triazolopyridyl, pyrazolopyrimidyl, dihydro-pyrimidopyridazinyl and dihydro-[1,4]dioxinopyridazinyl; and most preferably, the 5-12 membered heteroaryl is selected from pyridyl and pyridazinyl;
wherein the 5-12 membered heteroaryl is optionally substituted with one or more groups independently selected from halogen, —OH, oxo, —CN, —NH₂, —CONH₂, C₁₋₆ alkyl, C₂₋₆ alkenyl, C₂₋₆ alkynyl, C₁₋₆ haloalkyl, —(C₁₋₆ alkyl)-OH, —O—(C₁₋₆ alkyl) and C₃₋₈ cycloalkyl; preferably, the 5-12 membered heteroaryl is optionally substituted with one or more groups independently selected from halogen, —OH, —CN, —CONH₂, C₁₋₆ alkyl, C₂₋₆ alkenyl, C₂₋₆ alkynyl, C₁₋₆ haloalkyl, —(C₁₋₆ alkyl)-OH, —O—(C₁₋₆ alkyl) and C₃₋₈ cycloalkyl;
10) —CONRₐR_{b}, wherein Rₐ and R_{b} are each independently selected from hydrogen, C₁₋₆ alkyl, —(C₁₋₆ alkyl)ₘ-(C₃₋₆ cycloalkyl), —(C₁₋₆ alkyl)ₘ-(4-6 membered heterocyclyl), —(C₁₋₆ alkyl)ₘ-phenyl and —(C₁₋₆ alkyl)ₘ-(5-6 membered heteroaryl), wherein the C₁₋₆ alkyl, C₃₋₆ cycloalkyl, 4-6 membered heterocyclyl, phenyl and 5-6 membered heteroaryl are each optionally substituted with one or more groups independently selected from halogen and —O—(C₁₋₆ alkyl);
11) —COR_{c}, wherein R_{c} is selected from C₁₋₆ alkyl, C₂₋₆ alkenyl, —(C₁₋₆ alkyl)ₘ-(C₃₋₈ cycloalkyl), —(C₁₋₆ alkyl)ₘ-(4-6 membered heterocyclyl), —(C₁₋₆ alkyl)ₘ-phenyl and —(C₁₋₆ alkyl)ₘ-(5-10 membered heteroaryl), wherein the C₁₋₆ alkyl, C₃₋₈ cycloalkyl, 4-6 membered heterocyclyl, phenyl and 5-10 membered heteroaryl are each optionally substituted with one or more groups independently selected from halogen, —OH, —CN, C₁₋₆ alkyl, C₁₋₆ haloalkyl, —O—(C₁₋₆ alkyl), —NH(C₃₋₆ cycloalkyl), —CO(C₂₋₆ alkenyl) and —CON(C₁₋₆ alkyl)₂;
12) —COORₑ, wherein Rₑ is selected from hydrogen and C₁₋₆ alkyl;
13) —NRₐR_{b}, wherein Rₐ and R_{b} are each independently selected from hydrogen and C₁₋₆ alkyl, wherein the C₁₋₆ alkyl is optionally substituted with one or more groups independently selected from —CN and —O—(C₁₋₆ alkyl);
14) —NR_{d}COR_{c}, wherein R_{d} is selected from hydrogen and C₁₋₆ alkyl; Rₑ is C₁₋₆ alkyl, which is optionally substituted with one or more groups independently selected from —CN and —O—(C₁₋₆ alkyl);
15) —NR_{d}S(O)ₙR_{f}, wherein R_{d} is hydrogen; R_{f} is —CH₃;
16) —S(O)ₙR_{f}, wherein R_{f} is —CH₃;
L is CH₂;
p is 0, 1, 2 or 3;
m is 0 or 1; and
n is 1 or 2; preferably, n is 2.

17. The compound or the pharmaceutically acceptable salt, the solvate, the racemic mixture, the enantiomer, the diastereomer or the tautomer thereof, or the deuterate thereof according to claim 1, wherein the compound is a compound of formula (I-4): 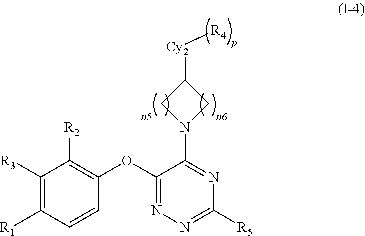
wherein
n5 and n6 are each independently selected from 1 and 2; preferably, both n5 and n6 are 1;
R₁ is halogen; preferably, R₁ is F;
R₂ is —CON(C₁₋₆ alkyl)₂;
R₃ is hydrogen;
R₅ is hydrogen;
Cy₂ is selected from C₃₋₈ cycloalkyl and 4-8 membered heterocyclyl; preferably, Cy₂ is selected from cyclopropyl and pyrrolidyl; more preferably, Cy₂ is selected from 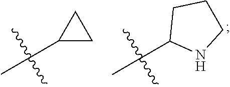
and most preferably, Cy₂ is 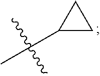 [SMILES: *C1CC1]
R₄ is independently selected from —(C₁₋₆ alkyl)ₘ-(5-12 membered heteroaryl) and —NRₐR_{b}, wherein the 5-12 membered heteroaryl is optionally substituted with one or more groups independently selected from —CN and C₁₋₆ alkyl; preferably, R₄ is independently selected from:
1) —(C₁₋₆ alkyl)-indolyl, wherein the indolyl is optionally substituted with one or more groups independently selected from —CN and C₁₋₆ alkyl;
2) —NRₐR_{b}, wherein Rₐ and R_{b} are each independently selected from hydrogen, C₁₋₆ alkyl and —(C₁₋₆ alkyl)ₘ-(5-10 membered heteroaryl), wherein the C₁₋₆ alkyl and 5-10 membered heteroaryl are each optionally substituted with one or more groups independently selected from —OH, —CN, C₁₋₆ alkyl and —(C₁₋₆ alkyl)-OH, provided that, R₄ is not —NH(6-membered nitrogen-containing heteroaryl);
p is 0, 1 or 2; preferably, p is 1; and
m is 0 or 1.

18. A compound or the pharmaceutically acceptable salt, the solvate, the racemic mixture, the enantiomer, the diastereomer or the tautomer thereof, or the deuterate thereof, which is selected from:

| No. | Structural formula |
|---|---|
| 1 | 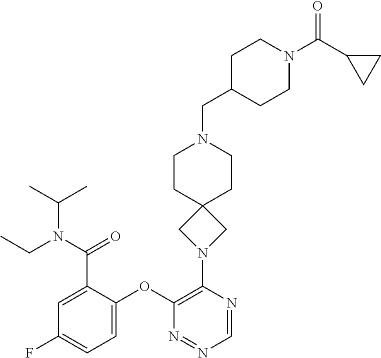 [SMILES: CCN(C(=O)c1cc(F)ccc1Oc1nncnc1N1CC2(CCN(CC3CCN(C(=O)C4CC4)CC3)CC2)C1)C(C)C] |
| 2 | 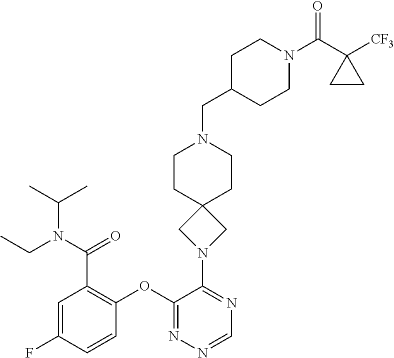 [SMILES: CCN(C(=O)c1cc(F)ccc1Oc1nncnc1N1CC2(CCN(CC3CCN(C(=O)C4(C(F)(F)F)CC4)CC3)CC2)C1)C(C)C] |
| 3 | 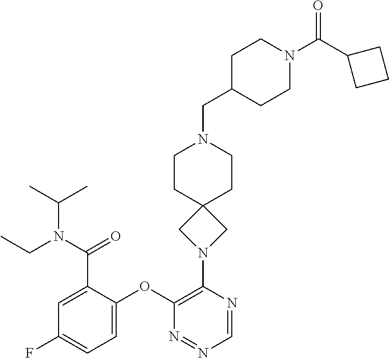 [SMILES: CCN(C(=O)c1cc(F)ccc1Oc1nncnc1N1CC2(CCN(CC3CCN(C(=O)C4CCC4)CC3)CC2)C1)C(C)C] |
| 4 | 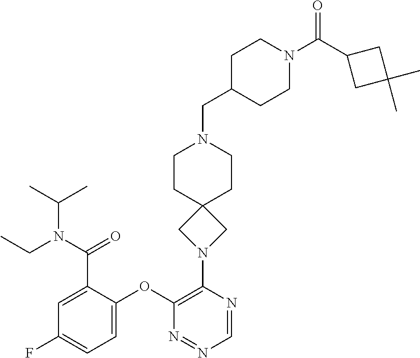 [SMILES: CCN(C(=O)c1cc(F)ccc1Oc1nncnc1N1CC2(CCN(CC3CCN(C(=O)C4CC(C)(C)C4)CC3)CC2)C1)C(C)C] |
| 5 | 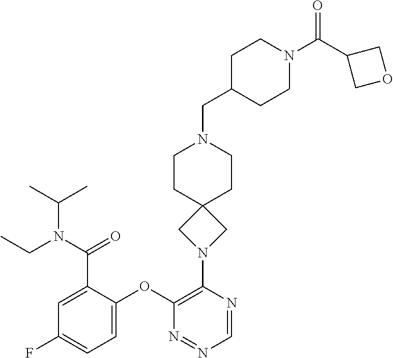 [SMILES: CCN(C(=O)c1cc(F)ccc1Oc1nncnc1N1CC2(CCN(CC3CCN(C(=O)C4COC4)CC3)CC2)C1)C(C)C] |
| 6 | 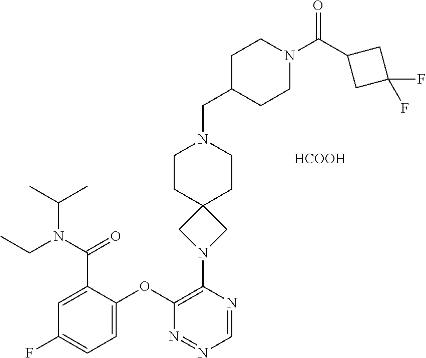 [SMILES: CCN(C(=O)c1cc(F)ccc1Oc1nncnc1N1CC2(CCN(CC3CCN(C(=O)C4CC(F)(F)C4)CC3)CC2)C1)C(C)C ; "HCOOH"] |
| 7 | 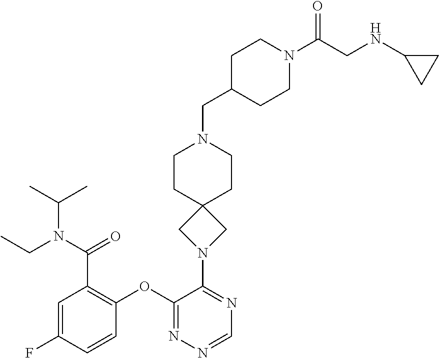 [SMILES: CCN(C(=O)c1cc(F)ccc1Oc1nncnc1N1CC2(CCN(CC3CCN(C(=O)CNC4CC4)CC3)CC2)C1)C(C)C] |
| 8 | 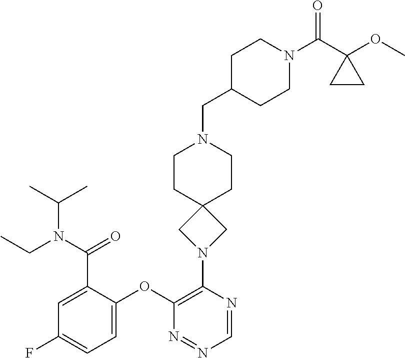 [SMILES: CCN(C(=O)c1cc(F)ccc1Oc1nncnc1N1CC2(CCN(CC3CCN(C(=O)C4(OC)CC4)CC3)CC2)C1)C(C)C] |
| 9 | 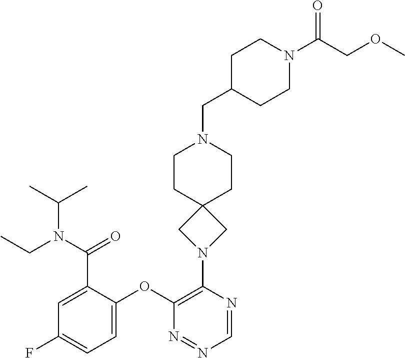 [SMILES: CCN(C(=O)c1cc(F)ccc1Oc1nncnc1N1CC2(CCN(CC3CCN(C(=O)COC)CC3)CC2)C1)C(C)C] |
| 10 | 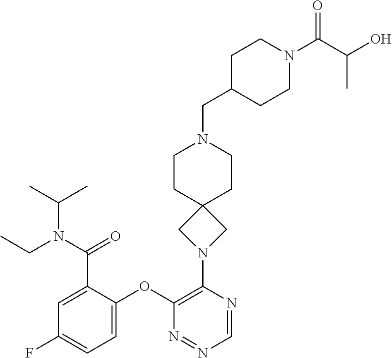 [SMILES: CCN(C(=O)c1cc(F)ccc1Oc1nncnc1N1CC2(CCN(CC3CCN(C(=O)C(C)O)CC3)CC2)C1)C(C)C] |
| 11 | 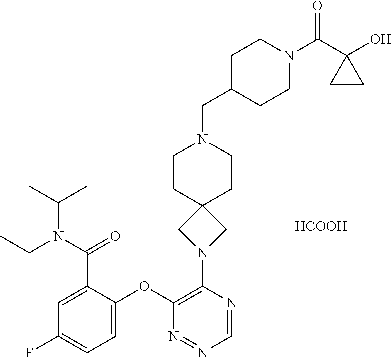 [SMILES: CCN(C(=O)c1cc(F)ccc1Oc1nncnc1N1CC2(CCN(CC3CCN(C(=O)C4(O)CC4)CC3)CC2)C1)C(C)C ; "HCOOH"] |
| 12 | 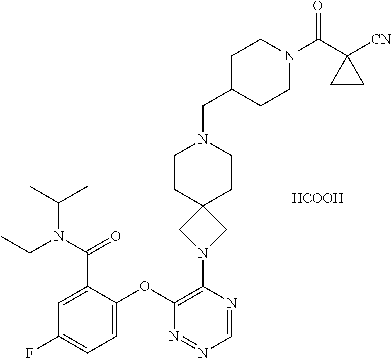 [SMILES: CCN(C(=O)c1cc(F)ccc1Oc1nncnc1N1CC2(CCN(CC3CCN(C(=O)C4(C#N)CC4)CC3)CC2)C1)C(C)C ; "HCOOH"] |
| 13 | 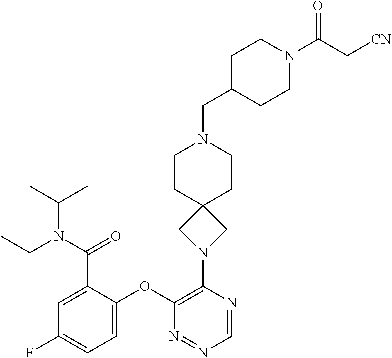 [SMILES: CCN(C(=O)c1cc(F)ccc1Oc1nncnc1N1CC2(CCN(CC3CCN(C(=O)CC#N)CC3)CC2)C1)C(C)C] |
| 14 | 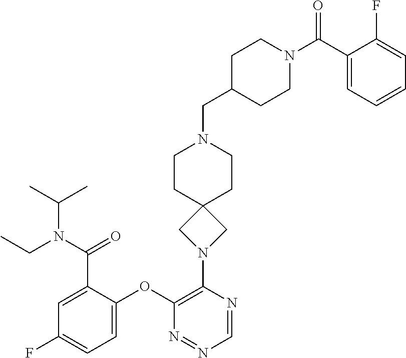 [SMILES: CCN(C(=O)c1cc(F)ccc1Oc1nncnc1N1CC2(CCN(CC3CCN(C(=O)c4ccccc4F)CC3)CC2)C1)C(C)C] |
| 15 | 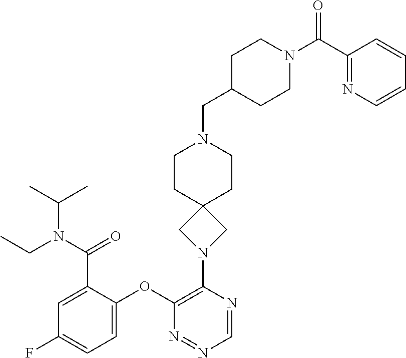 [SMILES: CCN(C(=O)c1cc(F)ccc1Oc1nncnc1N1CC2(CCN(CC3CCN(C(=O)c4ccccn4)CC3)CC2)C1)C(C)C] |
| 16 | 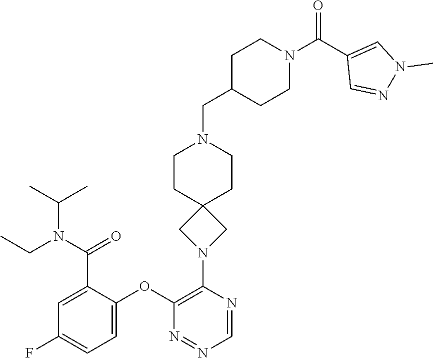 [SMILES: CCN(C(=O)c1cc(F)ccc1Oc1nncnc1N1CC2(CCN(CC3CCN(C(=O)c4cnn(C)c4)CC3)CC2)C1)C(C)C] |
| 17 | 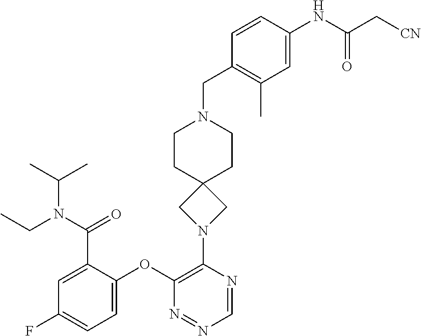 [SMILES: CCN(C(=O)c1cc(F)ccc1Oc1nncnc1N1CC2(CCN(Cc3ccc(NC(=O)CC#N)cc3C)CC2)C1)C(C)C] |
| 18 | 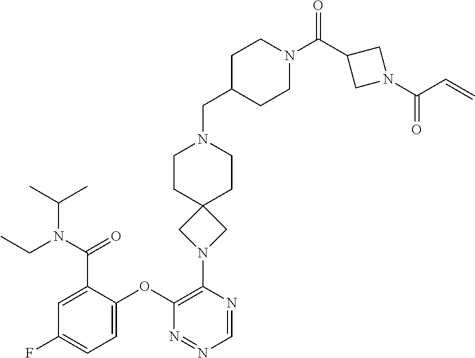 [SMILES: C=CC(=O)N1CC(C(=O)N2CCC(CN3CCC4(CC3)CN(c3ncnnc3Oc3ccc(F)cc3C(=O)N(CC)C(C)C)C4)CC2)C1] |
| 19 | 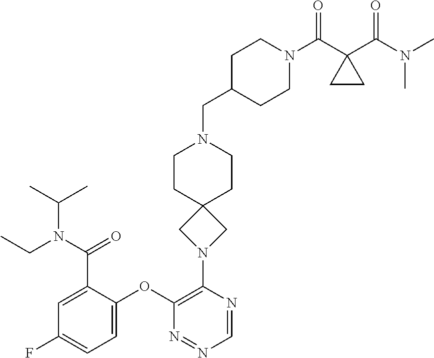 [SMILES: CCN(C(=O)c1cc(F)ccc1Oc1nncnc1N1CC2(CCN(CC3CCN(C(=O)C4(C(=O)N(C)C)CC4)CC3)CC2)C1)C(C)C] |
| 20 | 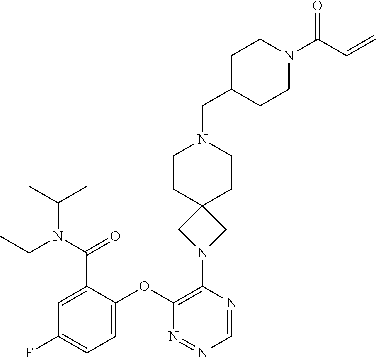 [SMILES: C=CC(=O)N1CCC(CN2CCC3(CC2)CN(c2ncnnc2Oc2ccc(F)cc2C(=O)N(CC)C(C)C)C3)CC1] |
| 21 | 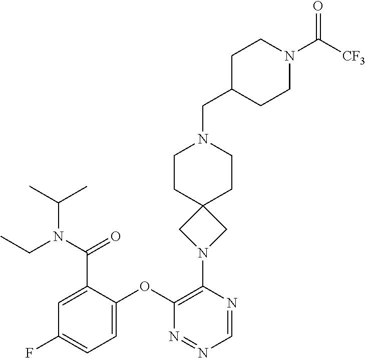 [SMILES: CCN(C(=O)c1cc(F)ccc1Oc1nncnc1N1CC2(CCN(CC3CCN(C(=O)C(F)(F)F)CC3)CC2)C1)C(C)C] |
| 22 | 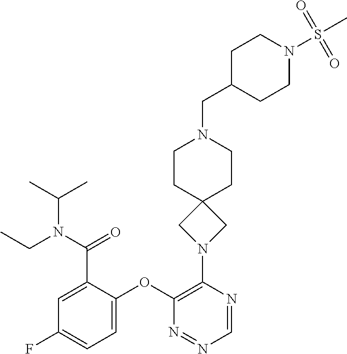 [SMILES: CCN(C(=O)c1cc(F)ccc1Oc1nncnc1N1CC2(CCN(CC3CCN(S(C)(=O)=O)CC3)CC2)C1)C(C)C] |
| 23 | 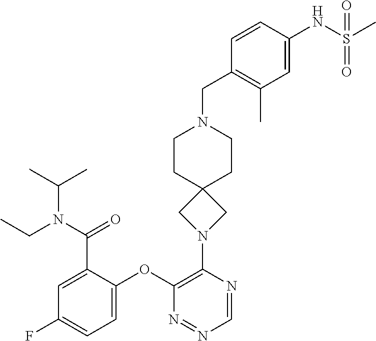 [SMILES: CCN(C(=O)c1cc(F)ccc1Oc1nncnc1N1CC2(CCN(Cc3ccc(NS(C)(=O)=O)cc3C)CC2)C1)C(C)C] |
| 24 | 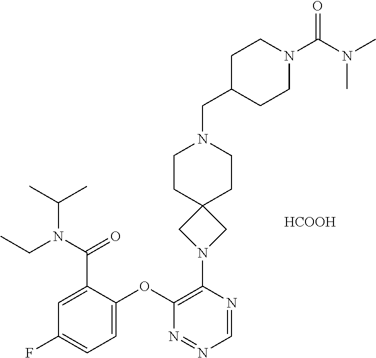 [SMILES: CCN(C(=O)c1cc(F)ccc1Oc1nncnc1N1CC2(CCN(CC3CCN(C(=O)N(C)C)CC3)CC2)C1)C(C)C ; "HCOOH"] |
| 25 | 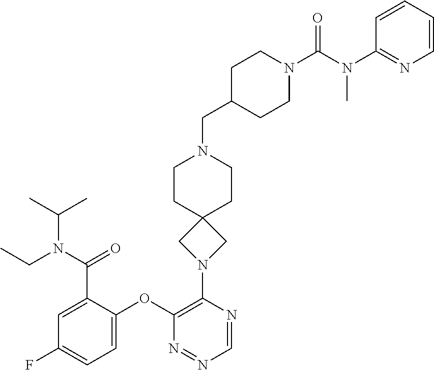 [SMILES: CCN(C(=O)c1cc(F)ccc1Oc1nncnc1N1CC2(CCN(CC3CCN(C(=O)N(C)c4ccccn4)CC3)CC2)C1)C(C)C] |
| 26 | 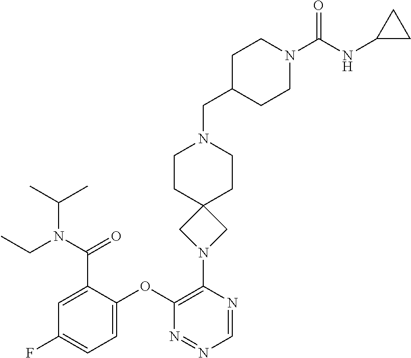 [SMILES: CCN(C(=O)c1cc(F)ccc1Oc1nncnc1N1CC2(CCN(CC3CCN(C(=O)NC4CC4)CC3)CC2)C1)C(C)C] |
| 27 | 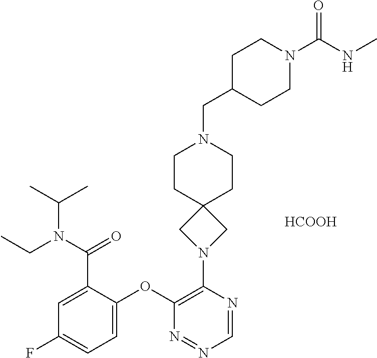 [SMILES: CCN(C(=O)c1cc(F)ccc1Oc1nncnc1N1CC2(CCN(CC3CCN(C(=O)NC)CC3)CC2)C1)C(C)C ; "HCOOH"] |
| 28 | 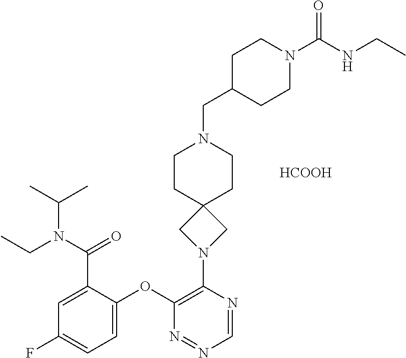 [SMILES: CCNC(=O)N1CCC(CN2CCC3(CC2)CN(c2ncnnc2Oc2ccc(F)cc2C(=O)N(CC)C(C)C)C3)CC1 ; "HCOOH"] |
| 29 | 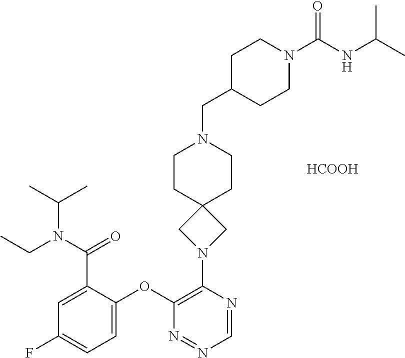 [SMILES: CCN(C(=O)c1cc(F)ccc1Oc1nncnc1N1CC2(CCN(CC3CCN(C(=O)NC(C)C)CC3)CC2)C1)C(C)C ; "HCOOH"] |
| 30 | 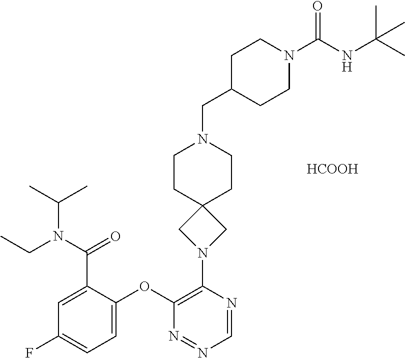 [SMILES: CCN(C(=O)c1cc(F)ccc1Oc1nncnc1N1CC2(CCN(CC3CCN(C(=O)NC(C)(C)C)CC3)CC2)C1)C(C)C ; "HCOOH"] |
| 31 | 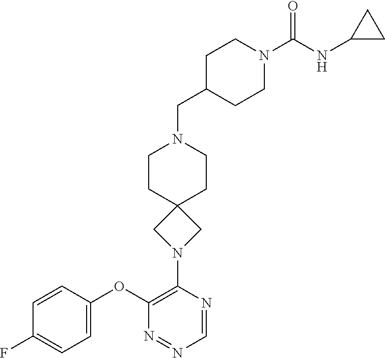 [SMILES: O=C(NC1CC1)N1CCC(CN2CCC3(CC2)CN(c2ncnnc2Oc2ccc(F)cc2)C3)CC1] |
| 32 | 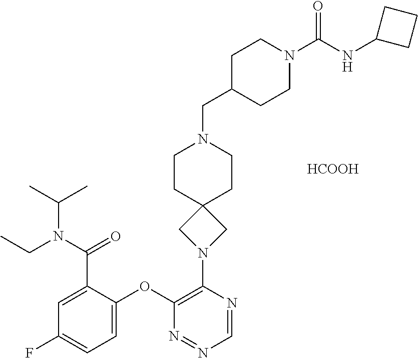 [SMILES: CCN(C(=O)c1cc(F)ccc1Oc1nncnc1N1CC2(CCN(CC3CCN(C(=O)NC4CCC4)CC3)CC2)C1)C(C)C ; "HCOOH"] |
| 33 | 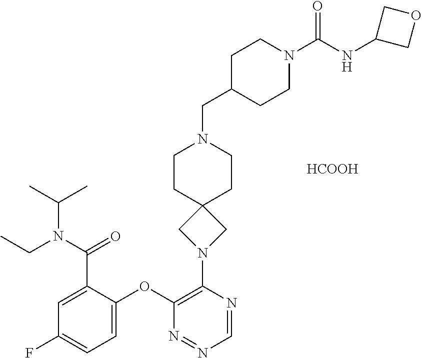 [SMILES: CCN(C(=O)c1cc(F)ccc1Oc1nncnc1N1CC2(CCN(CC3CCN(C(=O)NC4COC4)CC3)CC2)C1)C(C)C ; "HCOOH"] |
| 34 | 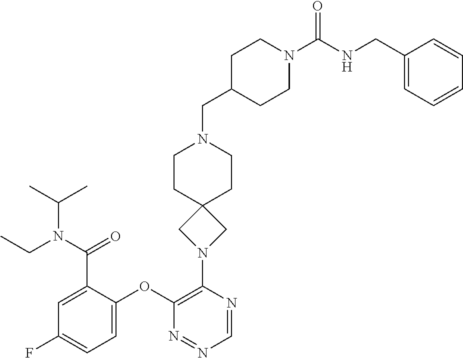 [SMILES: CCN(C(=O)c1cc(F)ccc1Oc1nncnc1N1CC2(CCN(CC3CCN(C(=O)NCc4ccccc4)CC3)CC2)C1)C(C)C] |
| 35 | 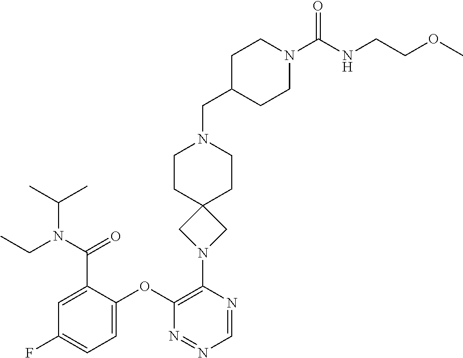 [SMILES: CCN(C(=O)c1cc(F)ccc1Oc1nncnc1N1CC2(CCN(CC3CCN(C(=O)NCCOC)CC3)CC2)C1)C(C)C] |
| 36 | 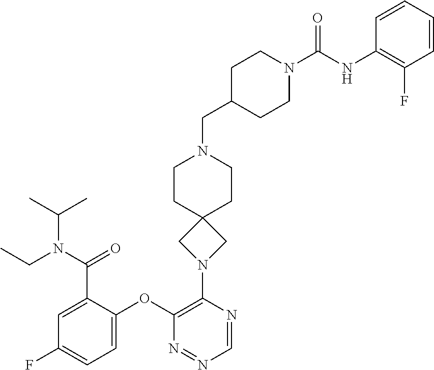 [SMILES: CCN(C(=O)c1cc(F)ccc1Oc1nncnc1N1CC2(CCN(CC3CCN(C(=O)Nc4ccccc4F)CC3)CC2)C1)C(C)C] |
| 37 | 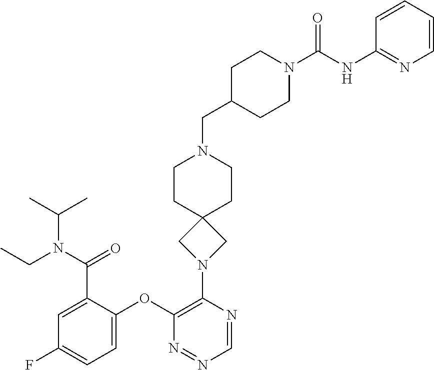 [SMILES: CCN(C(=O)c1cc(F)ccc1Oc1nncnc1N1CC2(CCN(CC3CCN(C(=O)Nc4ccccn4)CC3)CC2)C1)C(C)C] |
| 38 | 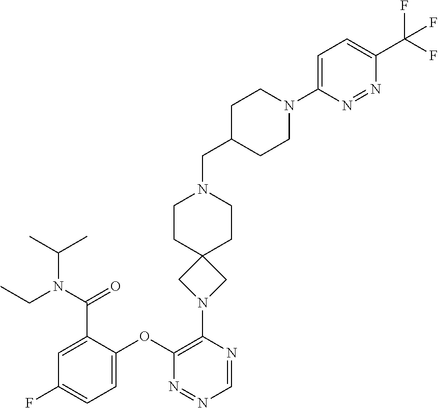 [SMILES: CCN(C(=O)c1cc(F)ccc1Oc1nncnc1N1CC2(CCN(CC3CCN(c4ccc(C(F)(F)F)nn4)CC3)CC2)C1)C(C)C] |
| 39 | 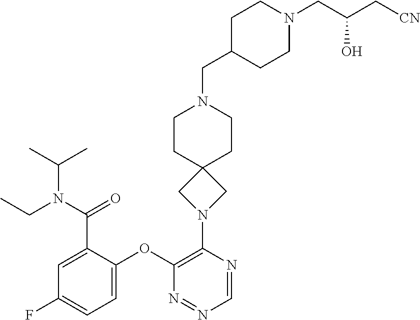 [SMILES: CCN(C(=O)c1cc(F)ccc1Oc1nncnc1N1CC2(CCN(CC3CCN(C[C@@H](O)CC#N)CC3)CC2)C1)C(C)C] |
| 40 | 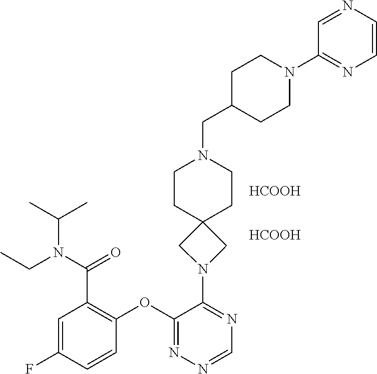 |
| 41 | 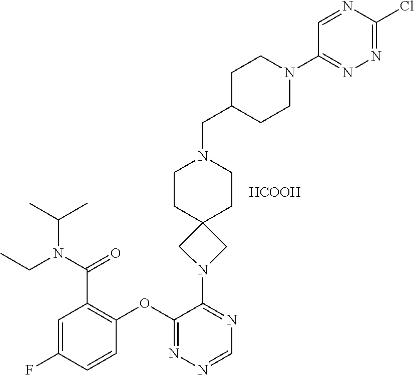 |
| 42 | 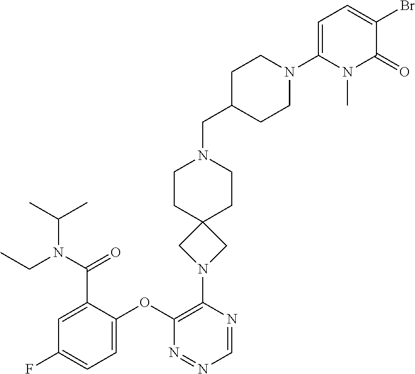 [SMILES: CCN(C(=O)c1cc(F)ccc1Oc1nncnc1N1CC2(CCN(CC3CCN(c4ccc(Br)c(=O)n4C)CC3)CC2)C1)C(C)C] |
| 43 | 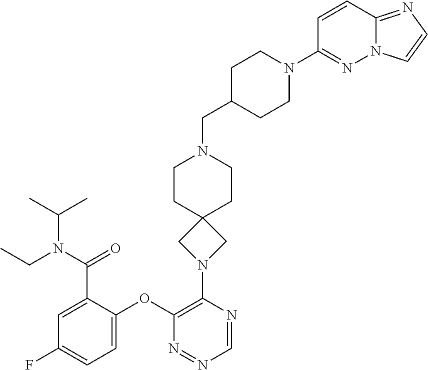 [SMILES: CCN(C(=O)c1cc(F)ccc1Oc1nncnc1N1CC2(CCN(CC3CCN(c4ccc5nccn5n4)CC3)CC2)C1)C(C)C] |
| 44 | 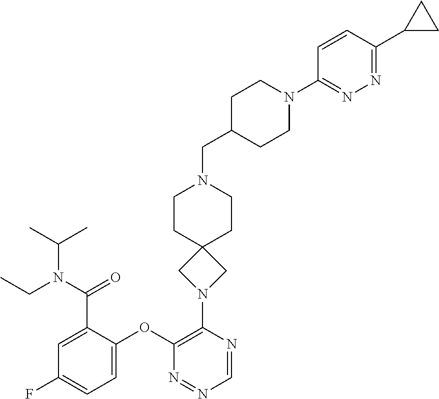 [SMILES: CCN(C(=O)c1cc(F)ccc1Oc1nncnc1N1CC2(CCN(CC3CCN(c4ccc(C5CC5)nn4)CC3)CC2)C1)C(C)C] |
| 45 | 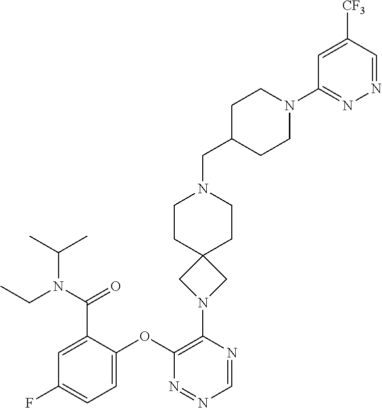 [SMILES: CCN(C(=O)c1cc(F)ccc1Oc1nncnc1N1CC2(CCN(CC3CCN(c4cc(C(F)(F)F)cnn4)CC3)CC2)C1)C(C)C] |
| 46 | 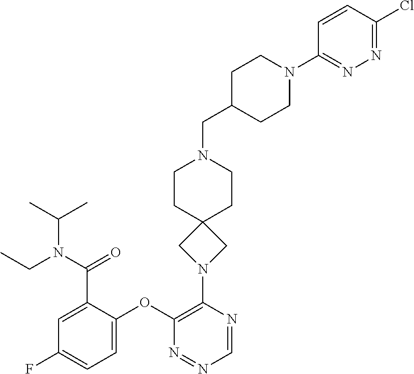 [SMILES: CCN(C(=O)c1cc(F)ccc1Oc1nncnc1N1CC2(CCN(CC3CCN(c4ccc(Cl)nn4)CC3)CC2)C1)C(C)C] |
| 47 | 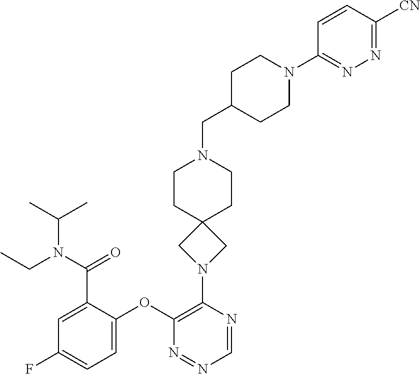 [SMILES: CCN(C(=O)c1cc(F)ccc1Oc1nncnc1N1CC2(CCN(CC3CCN(c4ccc(C#N)nn4)CC3)CC2)C1)C(C)C] |
| 48 | 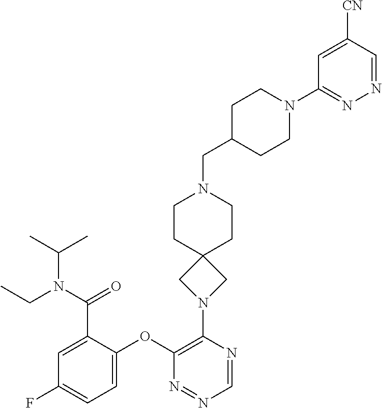 [SMILES: CCN(C(=O)c1cc(F)ccc1Oc1nncnc1N1CC2(CCN(CC3CCN(c4cc(C#N)cnn4)CC3)CC2)C1)C(C)C] |
| 49 | 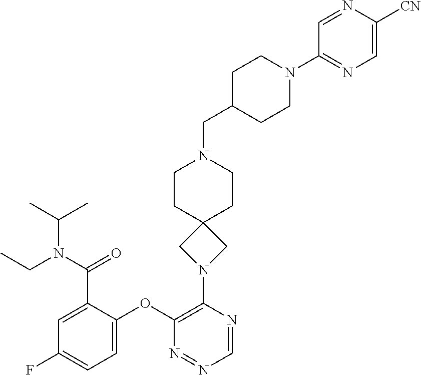 [SMILES: CCN(C(=O)c1cc(F)ccc1Oc1nncnc1N1CC2(CCN(CC3CCN(c4cnc(C#N)cn4)CC3)CC2)C1)C(C)C] |
| 50 | 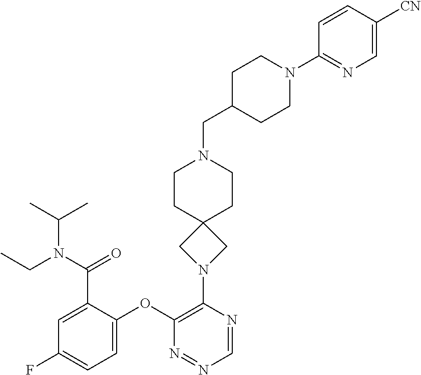 [SMILES: CCN(C(=O)c1cc(F)ccc1Oc1nncnc1N1CC2(CCN(CC3CCN(c4ccc(C#N)cn4)CC3)CC2)C1)C(C)C] |
| 51 | 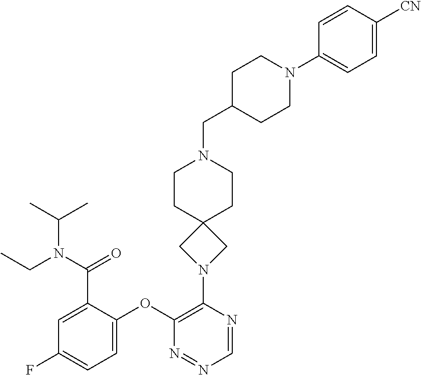 [SMILES: CCN(C(=O)c1cc(F)ccc1Oc1nncnc1N1CC2(CCN(CC3CCN(c4ccc(C#N)cc4)CC3)CC2)C1)C(C)C] |
| 52 | 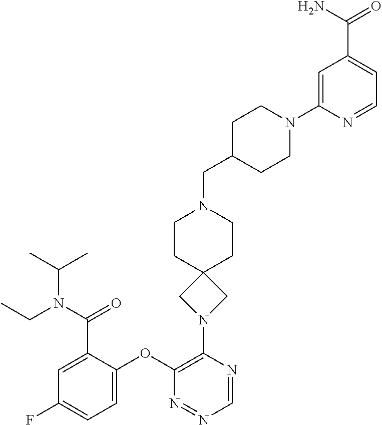 [SMILES: CCN(C(=O)c1cc(F)ccc1Oc1nncnc1N1CC2(CCN(CC3CCN(c4cc(C(N)=O)ccn4)CC3)CC2)C1)C(C)C] |
| 53 | 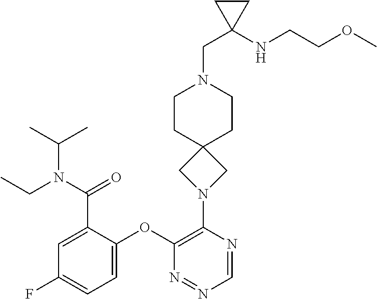 [SMILES: CCN(C(=O)c1cc(F)ccc1Oc1nncnc1N1CC2(CCN(CC3(NCCOC)CC3)CC2)C1)C(C)C] |
| 54 | 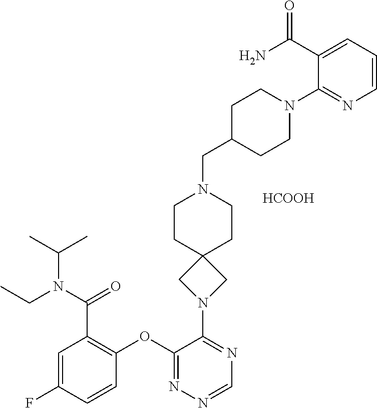 [SMILES: CCN(C(=O)c1cc(F)ccc1Oc1nncnc1N1CC2(CCN(CC3CCN(c4ncccc4C(N)=O)CC3)CC2)C1)C(C)C ; "HCOOH"] |
| 55 | 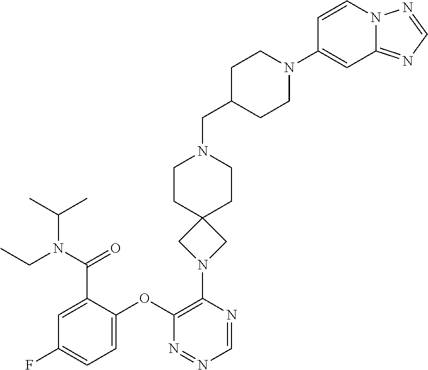 [SMILES: CCN(C(=O)c1cc(F)ccc1Oc1nncnc1N1CC2(CCN(CC3CCN(c4ccn5ncnc5c4)CC3)CC2)C1)C(C)C] |
| 56 | 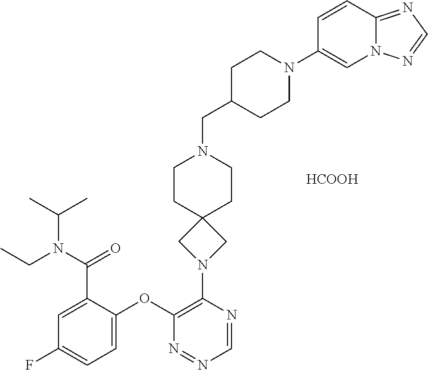 [SMILES: CCN(C(=O)c1cc(F)ccc1Oc1nncnc1N1CC2(CCN(CC3CCN(c4ccc5ncnn5c4)CC3)CC2)C1)C(C)C ; "HCOOH"] |
| 57 | 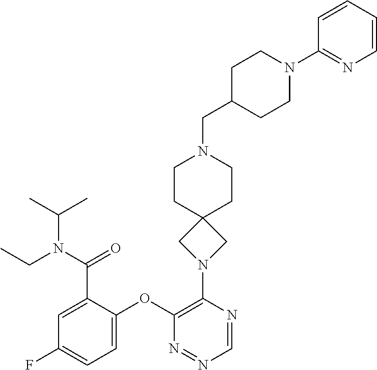 [SMILES: CCN(C(=O)c1cc(F)ccc1Oc1nncnc1N1CC2(CCN(CC3CCN(c4ccccn4)CC3)CC2)C1)C(C)C] |
| 58 | 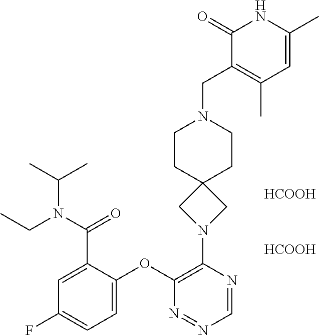 [SMILES: CCN(C(=O)c1cc(F)ccc1Oc1nncnc1N1CC2(CCN(Cc3c(C)cc(C)[nH]c3=O)CC2)C1)C(C)C ; "HCOOH" ; "HCOOH"] |
| 59 | 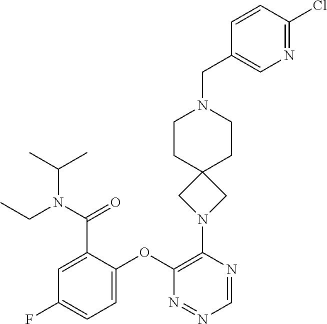 [SMILES: CCN(C(=O)c1cc(F)ccc1Oc1nncnc1N1CC2(CCN(Cc3ccc(Cl)nc3)CC2)C1)C(C)C] |
| 60 | 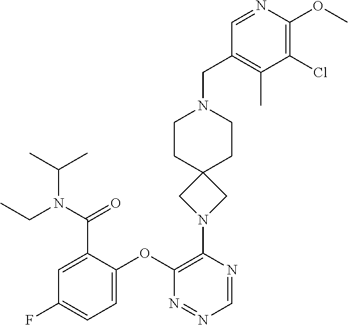 [SMILES: CCN(C(=O)c1cc(F)ccc1Oc1nncnc1N1CC2(CCN(Cc3cnc(OC)c(Cl)c3C)CC2)C1)C(C)C] |
| 61 | 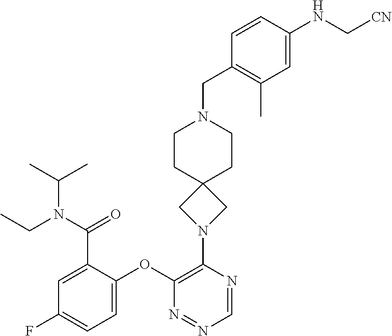 [SMILES: CCN(C(=O)c1cc(F)ccc1Oc1nncnc1N1CC2(CCN(Cc3ccc(NCC#N)cc3C)CC2)C1)C(C)C] |
| 62 | 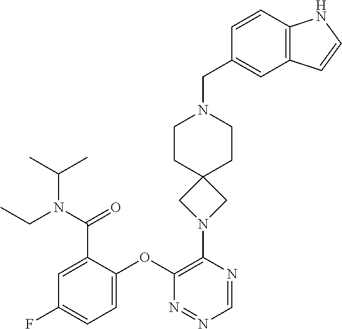 [SMILES: CCN(C(=O)c1cc(F)ccc1Oc1nncnc1N1CC2(CCN(Cc3ccc4[nH]ccc4c3)CC2)C1)C(C)C] |
| 63 | 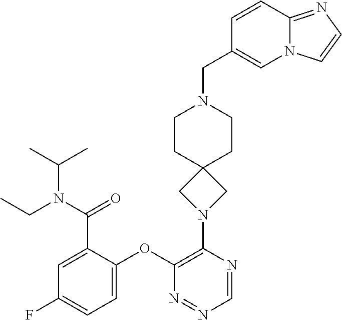 [SMILES: CCN(C(=O)c1cc(F)ccc1Oc1nncnc1N1CC2(CCN(Cc3ccc4nccn4c3)CC2)C1)C(C)C] |
| 64 | 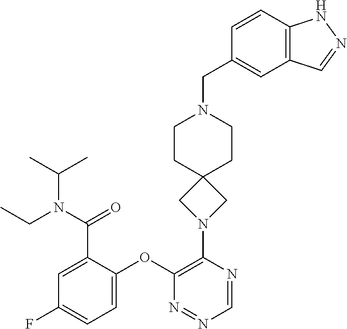 [SMILES: CCN(C(=O)c1cc(F)ccc1Oc1nncnc1N1CC2(CCN(Cc3ccc4[nH]ncc4c3)CC2)C1)C(C)C] |
| 65 | 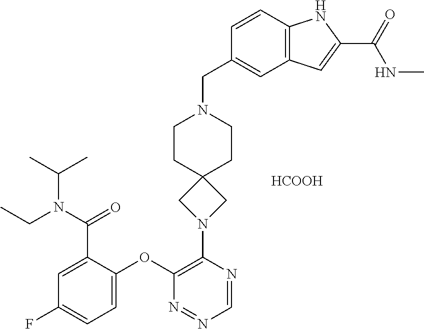 [SMILES: CCN(C(=O)c1cc(F)ccc1Oc1nncnc1N1CC2(CCN(Cc3ccc4[nH]c(C(=O)NC)cc4c3)CC2)C1)C(C)C ; "HCOOH"] |
| 66 | 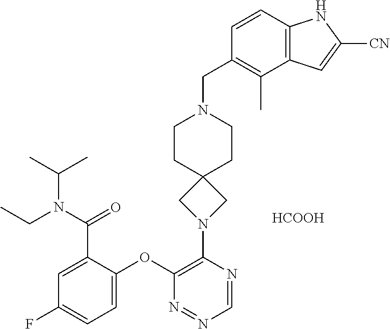 [SMILES: CCN(C(=O)c1cc(F)ccc1Oc1nncnc1N1CC2(CCN(Cc3ccc4[nH]c(C#N)cc4c3C)CC2)C1)C(C)C ; "HCOOH"] |
| 67 | 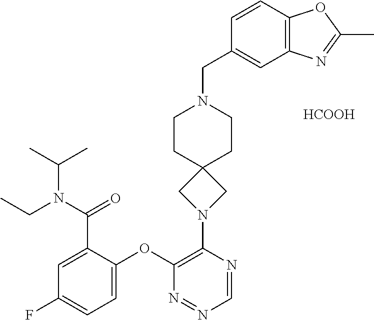 [SMILES: CCN(C(=O)c1cc(F)ccc1Oc1nncnc1N1CC2(CCN(Cc3ccc4oc(C)nc4c3)CC2)C1)C(C)C ; "HCOOH"] |
| 68 | 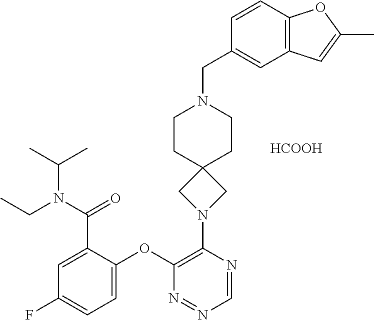 [SMILES: CCN(C(=O)c1cc(F)ccc1Oc1nncnc1N1CC2(CCN(Cc3ccc4oc(C)cc4c3)CC2)C1)C(C)C ; "HCOOH"] |
| 69 | 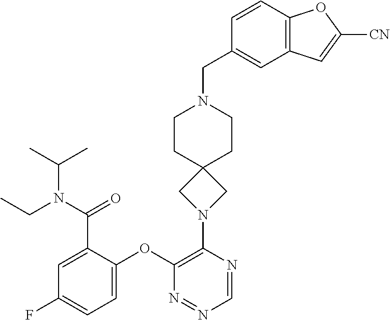 [SMILES: CCN(C(=O)c1cc(F)ccc1Oc1nncnc1N1CC2(CCN(Cc3ccc4oc(C#N)cc4c3)CC2)C1)C(C)C] |
| 70 | 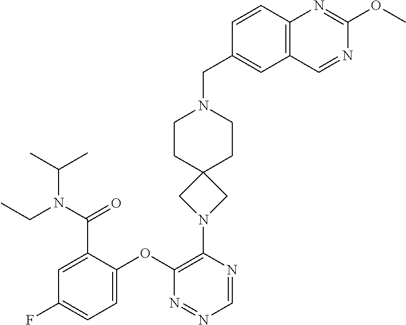 [SMILES: CCN(C(=O)c1cc(F)ccc1Oc1nncnc1N1CC2(CCN(Cc3ccc4nc(OC)ncc4c3)CC2)C1)C(C)C] |
| 71 | 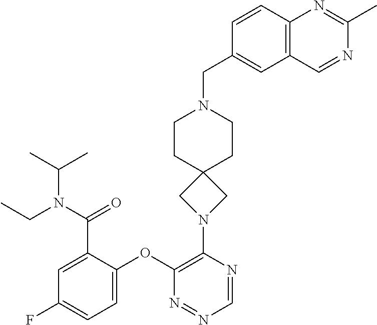 [SMILES: CCN(C(=O)c1cc(F)ccc1Oc1nncnc1N1CC2(CCN(Cc3ccc4nc(C)ncc4c3)CC2)C1)C(C)C] |
| 72 | 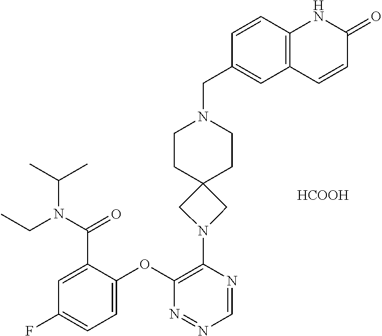 [SMILES: CCN(C(=O)c1cc(F)ccc1Oc1nncnc1N1CC2(CCN(Cc3ccc4[nH]c(=O)ccc4c3)CC2)C1)C(C)C ; "HCOOH"] |
| 73 | 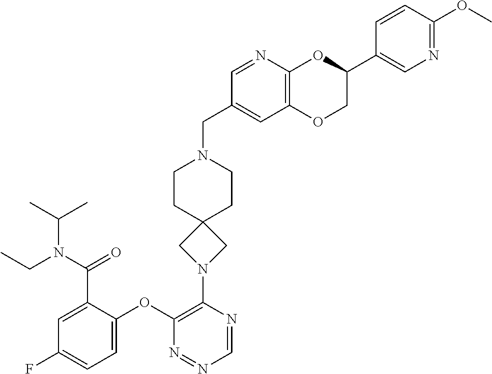 [SMILES: CCN(C(=O)c1cc(F)ccc1Oc1nncnc1N1CC2(CCN(Cc3cnc4c(c3)OC[C@H](c3ccc(OC)nc3)O4)CC2)C1)C(C)C] |
| 74 | 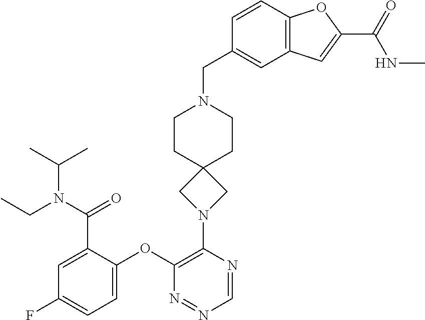 [SMILES: CCN(C(=O)c1cc(F)ccc1Oc1nncnc1N1CC2(CCN(Cc3ccc4oc(C(=O)NC)cc4c3)CC2)C1)C(C)C] |
| 75 | 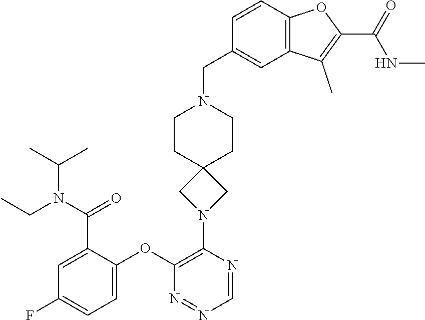 [SMILES: CCN(C(=O)c1cc(F)ccc1Oc1nncnc1N1CC2(CCN(Cc3ccc4oc(C(=O)NC)c(C)c4c3)CC2)C1)C(C)C] |
| 76 | 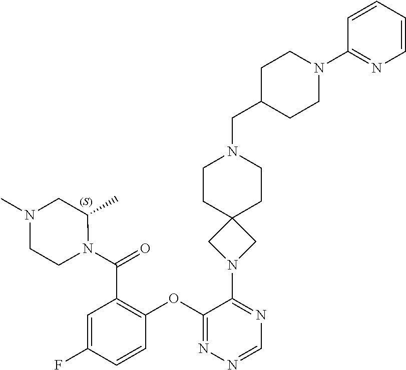 |
| 77 | 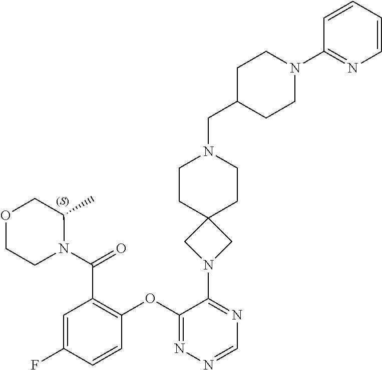 |
| 78 | 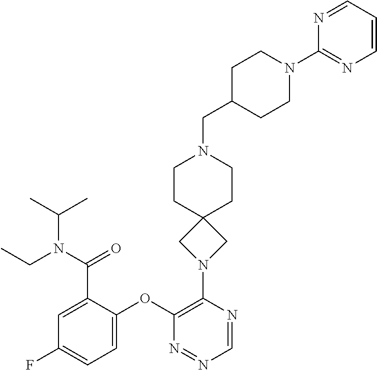 [SMILES: CCN(C(=O)c1cc(F)ccc1Oc1nncnc1N1CC2(CCN(CC3CCN(c4ncccn4)CC3)CC2)C1)C(C)C] |
| 79 | 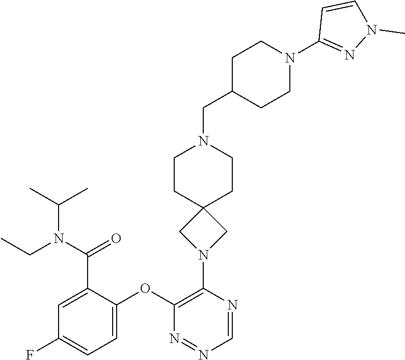 [SMILES: CCN(C(=O)c1cc(F)ccc1Oc1nncnc1N1CC2(CCN(CC3CCN(c4ccn(C)n4)CC3)CC2)C1)C(C)C] |
| 80 | 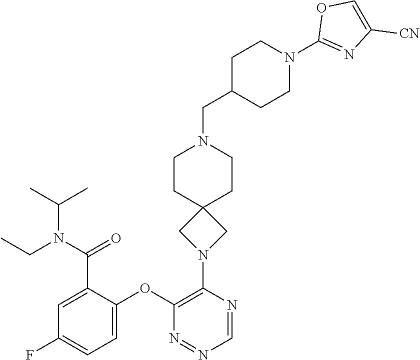 [SMILES: CCN(C(=O)c1cc(F)ccc1Oc1nncnc1N1CC2(CCN(CC3CCN(c4nc(C#N)co4)CC3)CC2)C1)C(C)C] |
| 81 | 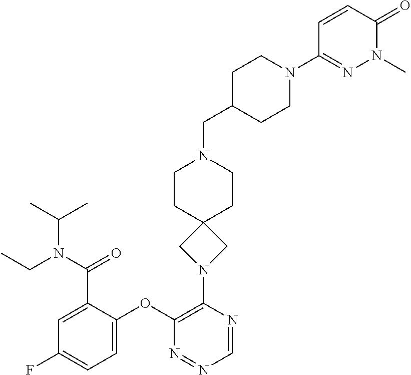 [SMILES: CCN(C(=O)c1cc(F)ccc1Oc1nncnc1N1CC2(CCN(CC3CCN(c4ccc(=O)n(C)n4)CC3)CC2)C1)C(C)C] |
| 82 | 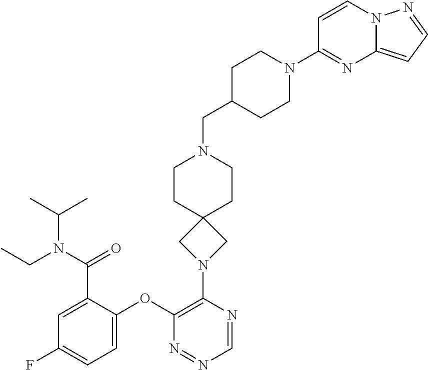 [SMILES: CCN(C(=O)c1cc(F)ccc1Oc1nncnc1N1CC2(CCN(CC3CCN(c4ccn5nccc5n4)CC3)CC2)C1)C(C)C] |
| 83 | 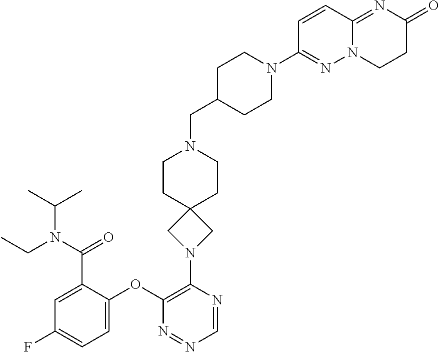 [SMILES: CCN(C(=O)c1cc(F)ccc1Oc1nncnc1N1CC2(CCN(CC3CCN(C4=NN5CCC(=O)N=C5C=C4)CC3)CC2)C1)C(C)C] |
| 84 | 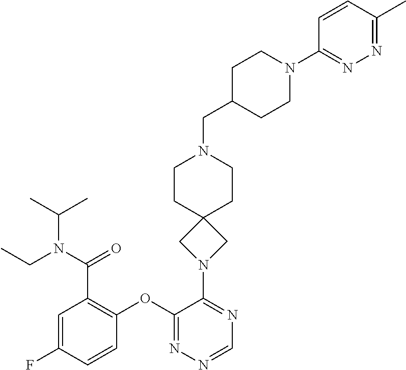 [SMILES: CCN(C(=O)c1cc(F)ccc1Oc1nncnc1N1CC2(CCN(CC3CCN(c4ccc(C)nn4)CC3)CC2)C1)C(C)C] |
| 85 | 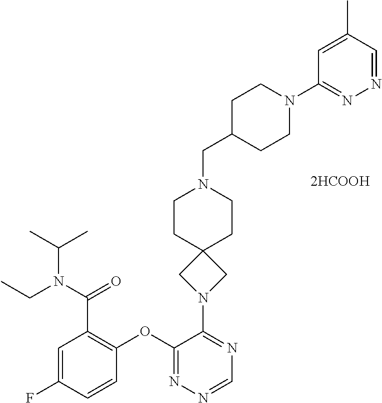 [SMILES: CCN(C(=O)c1cc(F)ccc1Oc1nncnc1N1CC2(CCN(CC3CCN(c4cc(C)cnn4)CC3)CC2)C1)C(C)C ; "2HCOOH"] |
| 86 | 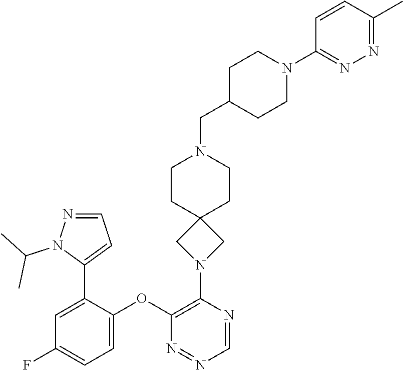 [SMILES: Cc1ccc(N2CCC(CN3CCC4(CC3)CN(c3ncnnc3Oc3ccc(F)cc3-c3ccnn3C(C)C)C4)CC2)nn1] |
| 87 | 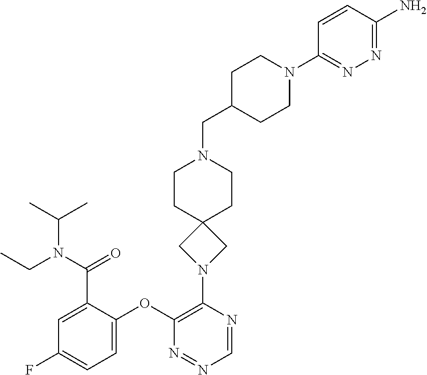 [SMILES: CCN(C(=O)c1cc(F)ccc1Oc1nncnc1N1CC2(CCN(CC3CCN(c4ccc(N)nn4)CC3)CC2)C1)C(C)C] |
| 88 | 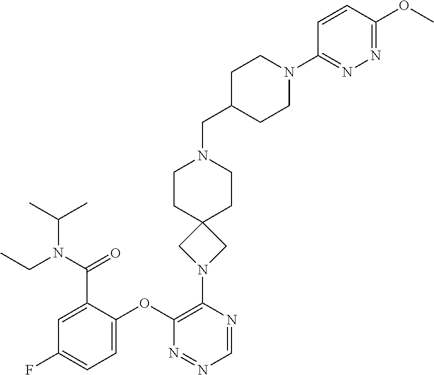 [SMILES: CCN(C(=O)c1cc(F)ccc1Oc1nncnc1N1CC2(CCN(CC3CCN(c4ccc(OC)nn4)CC3)CC2)C1)C(C)C] |
| 89 | 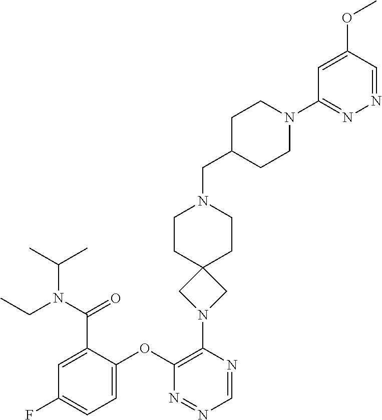 [SMILES: CCN(C(=O)c1cc(F)ccc1Oc1nncnc1N1CC2(CCN(CC3CCN(c4cc(OC)cnn4)CC3)CC2)C1)C(C)C] |
| 90 | 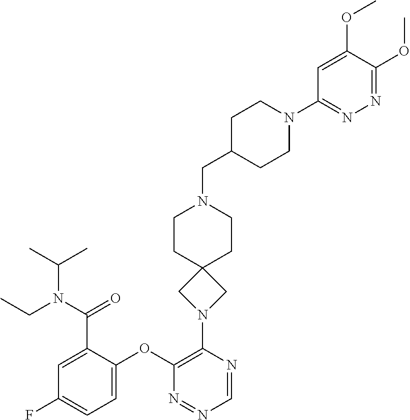 [SMILES: CCN(C(=O)c1cc(F)ccc1Oc1nncnc1N1CC2(CCN(CC3CCN(c4cc(OC)c(OC)nn4)CC3)CC2)C1)C(C)C] |
| 91 | 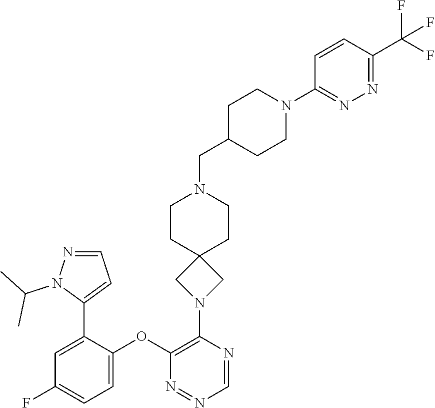 [SMILES: CC(C)n1nccc1-c1cc(F)ccc1Oc1nncnc1N1CC2(CCN(CC3CCN(c4ccc(C(F)(F)F)nn4)CC3)CC2)C1] |
| 92 | 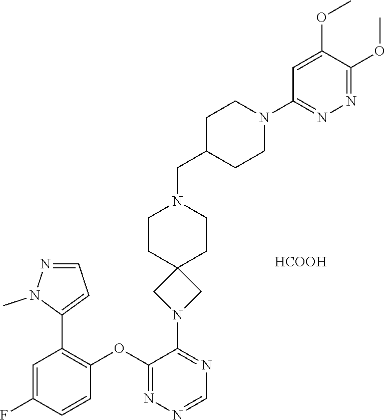 |
| 93 | 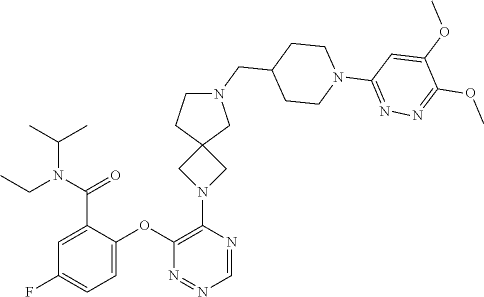 [SMILES: CCN(C(=O)c1cc(F)ccc1Oc1nncnc1N1CC2(CCN(CC3CCN(c4cc(OC)c(OC)nn4)CC3)C2)C1)C(C)C] |
| 94 | 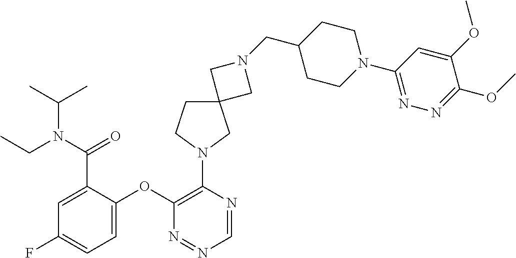 [SMILES: CCN(C(=O)c1cc(F)ccc1Oc1nncnc1N1CCC2(CN(CC3CCN(c4cc(OC)c(OC)nn4)CC3)C2)C1)C(C)C] |
| 95 | 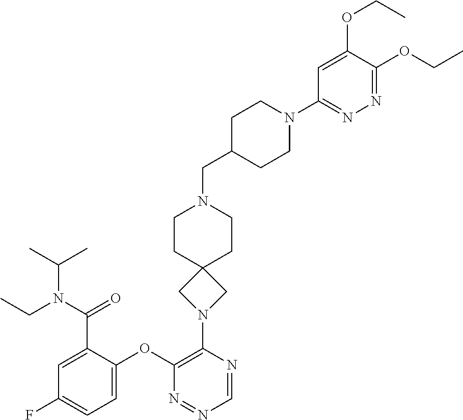 [SMILES: CCOc1cc(N2CCC(CN3CCC4(CC3)CN(c3ncnnc3Oc3ccc(F)cc3C(=O)N(CC)C(C)C)C4)CC2)nnc1OCC] |
| 96 | 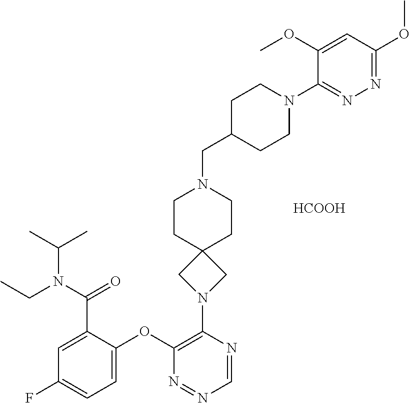 [SMILES: CCN(C(=O)c1cc(F)ccc1Oc1nncnc1N1CC2(CCN(CC3CCN(c4nnc(OC)cc4OC)CC3)CC2)C1)C(C)C ; "HCOOH"] |
| 97 | 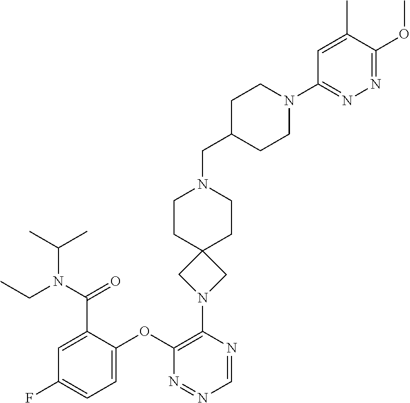 [SMILES: CCN(C(=O)c1cc(F)ccc1Oc1nncnc1N1CC2(CCN(CC3CCN(c4cc(C)c(OC)nn4)CC3)CC2)C1)C(C)C] |
| 98 | 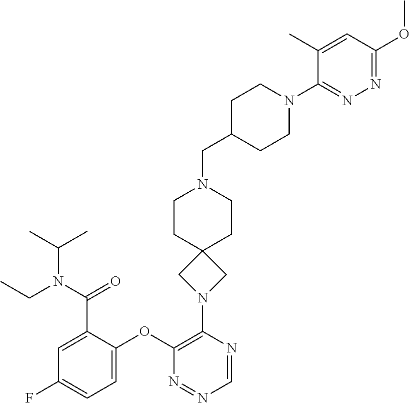 [SMILES: CCN(C(=O)c1cc(F)ccc1Oc1nncnc1N1CC2(CCN(CC3CCN(c4nnc(OC)cc4C)CC3)CC2)C1)C(C)C] |
| 99 | 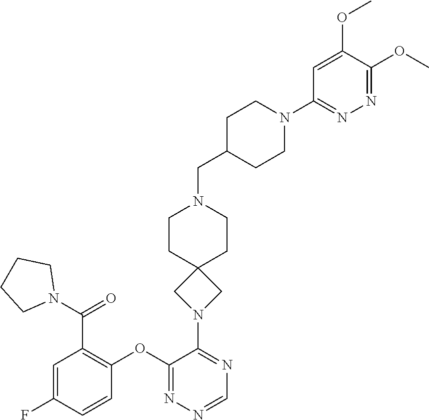 [SMILES: COc1cc(N2CCC(CN3CCC4(CC3)CN(c3ncnnc3Oc3ccc(F)cc3C(=O)N3CCCC3)C4)CC2)nnc1OC] |
| 100 | 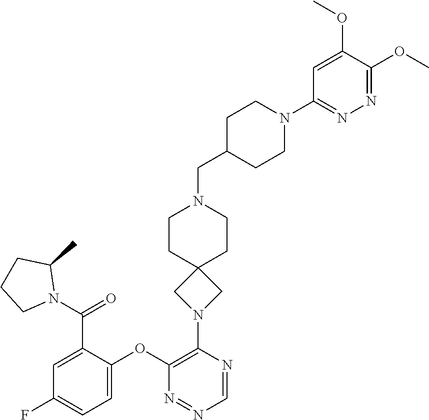 [SMILES: COc1cc(N2CCC(CN3CCC4(CC3)CN(c3ncnnc3Oc3ccc(F)cc3C(=O)N3CCC[C@H]3C)C4)CC2)nnc1OC] |
| 101 | 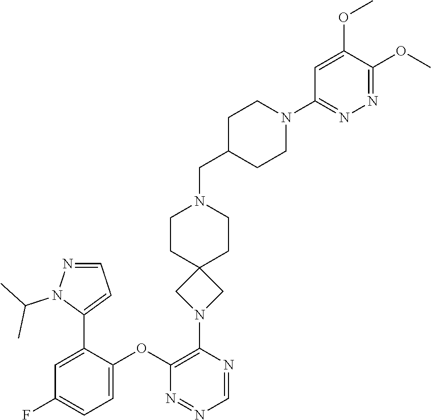 [SMILES: COc1cc(N2CCC(CN3CCC4(CC3)CN(c3ncnnc3Oc3ccc(F)cc3-c3ccnn3C(C)C)C4)CC2)nnc1OC] |
| 102 | 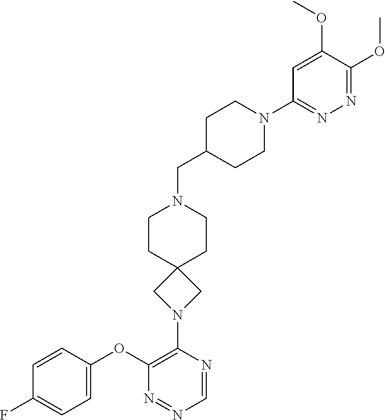 [SMILES: COc1cc(N2CCC(CN3CCC4(CC3)CN(c3ncnnc3Oc3ccc(F)cc3)C4)CC2)nnc1OC] |
| 103 | 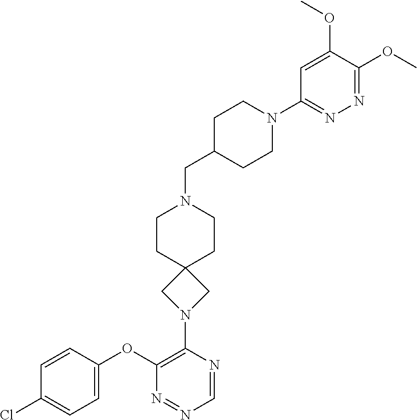 [SMILES: COc1cc(N2CCC(CN3CCC4(CC3)CN(c3ncnnc3Oc3ccc(Cl)cc3)C4)CC2)nnc1OC] |
| 104 | 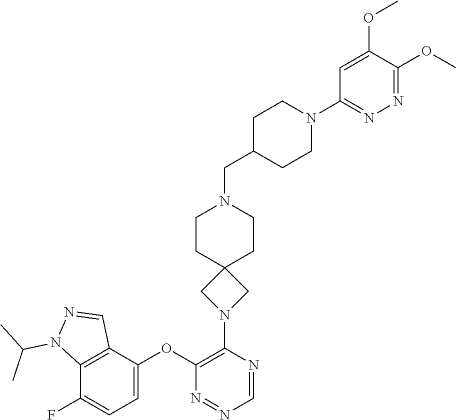 [SMILES: COc1cc(N2CCC(CN3CCC4(CC3)CN(c3ncnnc3Oc3ccc(F)c5c3cnn5C(C)C)C4)CC2)nnc1OC] |
| 105 | 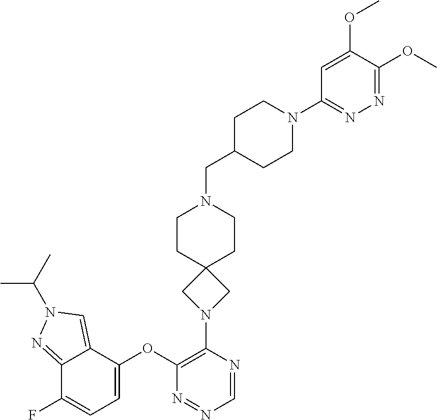 [SMILES: COc1cc(N2CCC(CN3CCC4(CC3)CN(c3ncnnc3Oc3ccc(F)c5nn(C(C)C)cc35)C4)CC2)nnc1OC] |
| 106 | 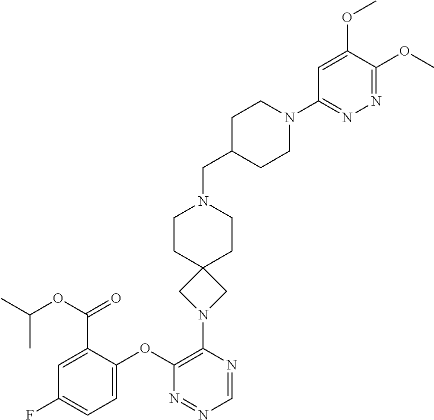 [SMILES: COc1cc(N2CCC(CN3CCC4(CC3)CN(c3ncnnc3Oc3ccc(F)cc3C(=O)OC(C)C)C4)CC2)nnc1OC] |
| 107 | 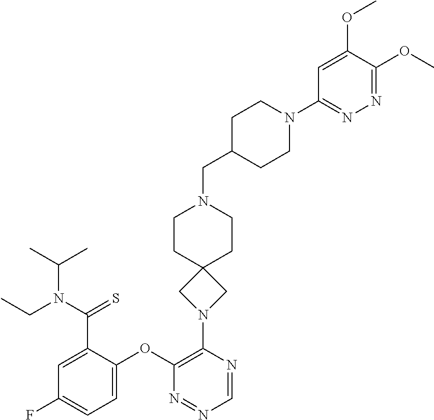 [SMILES: CCN(C(=S)c1cc(F)ccc1Oc1nncnc1N1CC2(CCN(CC3CCN(c4cc(OC)c(OC)nn4)CC3)CC2)C1)C(C)C] |
| 108 | 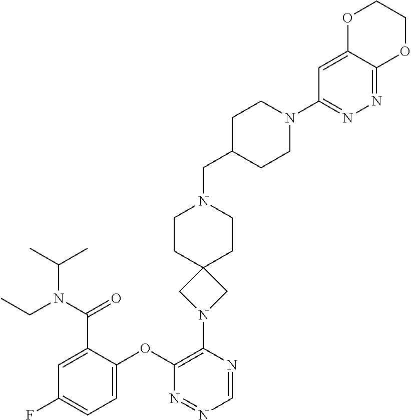 [SMILES: CCN(C(=O)c1cc(F)ccc1Oc1nncnc1N1CC2(CCN(CC3CCN(c4cc5c(nn4)OCCO5)CC3)CC2)C1)C(C)C] |
| 109 | 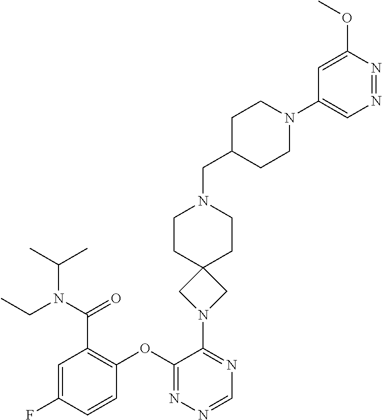 [SMILES: CCN(C(=O)c1cc(F)ccc1Oc1nncnc1N1CC2(CCN(CC3CCN(c4cnnc(OC)c4)CC3)CC2)C1)C(C)C] |
| 110 | 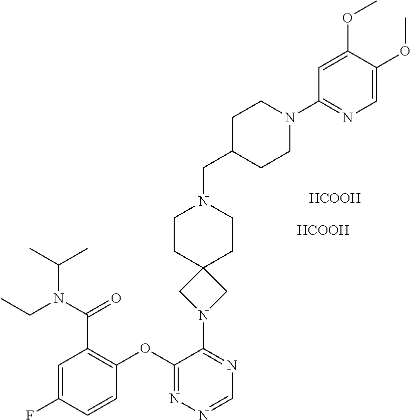 [SMILES: CCN(C(=O)c1cc(F)ccc1Oc1nncnc1N1CC2(CCN(CC3CCN(c4cc(OC)c(OC)cn4)CC3)CC2)C1)C(C)C ; "HCOOH" ; "HCOOH"] |
| 111 | 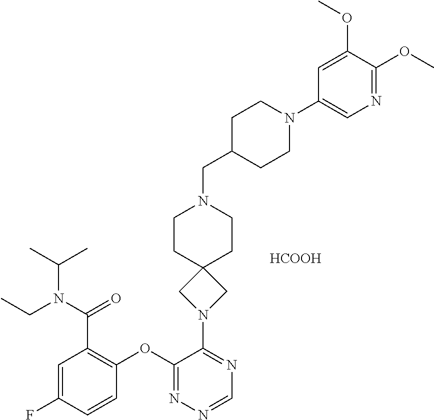 [SMILES: CCN(C(=O)c1cc(F)ccc1Oc1nncnc1N1CC2(CCN(CC3CCN(c4cnc(OC)c(OC)c4)CC3)CC2)C1)C(C)C ; "HCOOH"] |
| 112 | 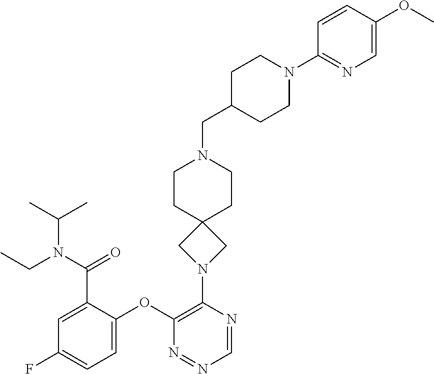 [SMILES: CCN(C(=O)c1cc(F)ccc1Oc1nncnc1N1CC2(CCN(CC3CCN(c4ccc(OC)cn4)CC3)CC2)C1)C(C)C] |
| 113 | 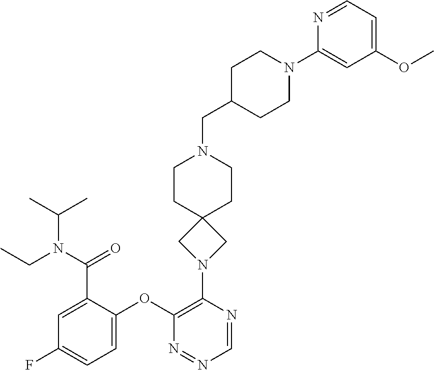 [SMILES: CCN(C(=O)c1cc(F)ccc1Oc1nncnc1N1CC2(CCN(CC3CCN(c4cc(OC)ccn4)CC3)CC2)C1)C(C)C] |
| 114 | 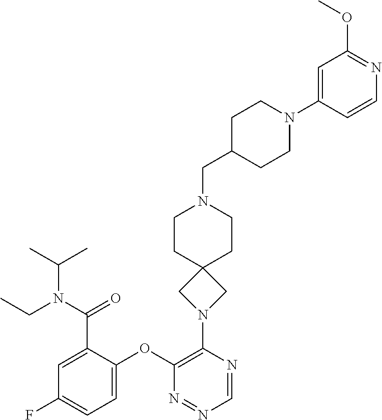 [SMILES: CCN(C(=O)c1cc(F)ccc1Oc1nncnc1N1CC2(CCN(CC3CCN(c4ccnc(OC)c4)CC3)CC2)C1)C(C)C] |
| 115 | 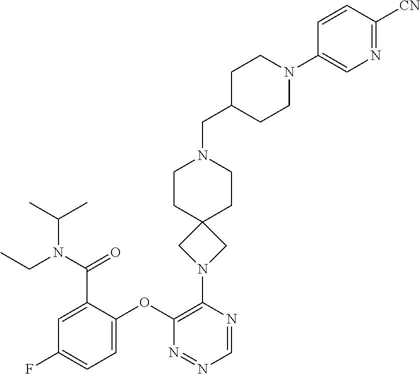 [SMILES: CCN(C(=O)c1cc(F)ccc1Oc1nncnc1N1CC2(CCN(CC3CCN(c4ccc(C#N)nc4)CC3)CC2)C1)C(C)C] |
| 116 | 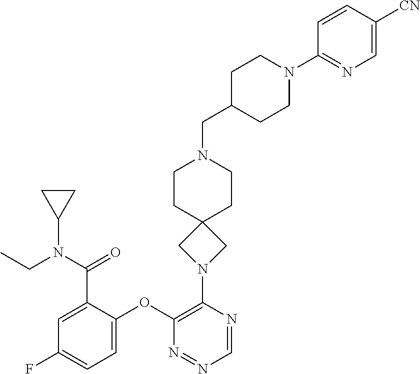 [SMILES: CCN(C(=O)c1cc(F)ccc1Oc1nncnc1N1CC2(CCN(CC3CCN(c4ccc(C#N)cn4)CC3)CC2)C1)C1CC1] |
| 117 | 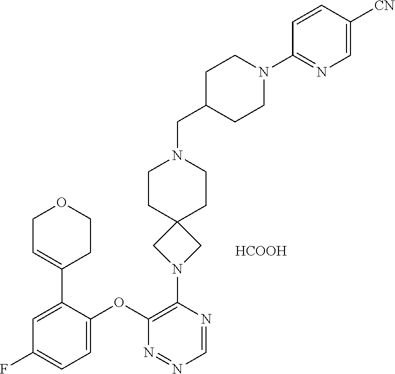 [SMILES: N#Cc1ccc(N2CCC(CN3CCC4(CC3)CN(c3ncnnc3Oc3ccc(F)cc3C3=CCOCC3)C4)CC2)nc1 ; "HCOOH"] |
| 118 | 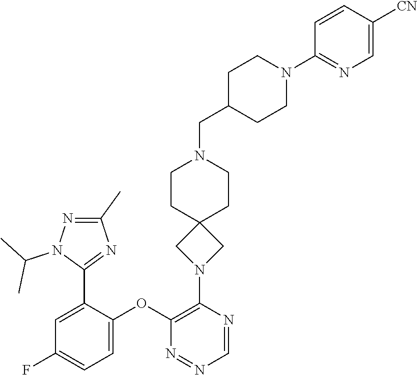 [SMILES: Cc1nc(-c2cc(F)ccc2Oc2nncnc2N2CC3(CCN(CC4CCN(c5ccc(C#N)cn5)CC4)CC3)C2)n(C(C)C)n1] |
| 119 | 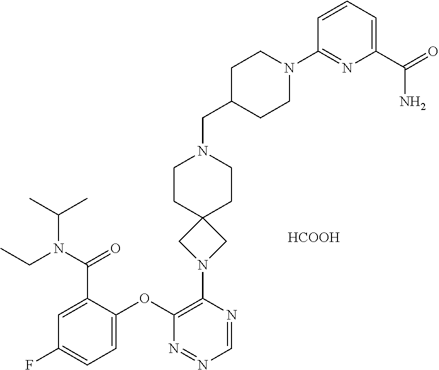 [SMILES: CCN(C(=O)c1cc(F)ccc1Oc1nncnc1N1CC2(CCN(CC3CCN(c4cccc(C(N)=O)n4)CC3)CC2)C1)C(C)C ; "HCOOH"] |
| 120 | 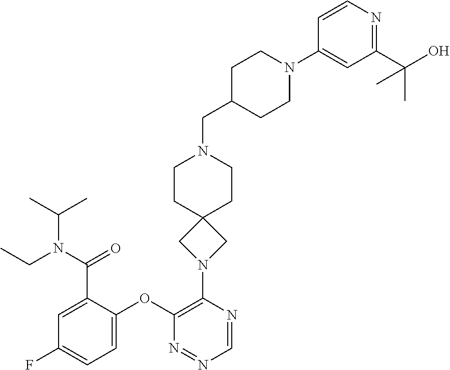 [SMILES: CCN(C(=O)c1cc(F)ccc1Oc1nncnc1N1CC2(CCN(CC3CCN(c4ccnc(C(C)(C)O)c4)CC3)CC2)C1)C(C)C] |
| 121 | 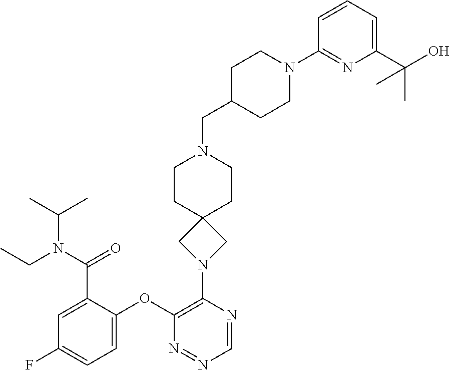 [SMILES: CCN(C(=O)c1cc(F)ccc1Oc1nncnc1N1CC2(CCN(CC3CCN(c4cccc(C(C)(C)O)n4)CC3)CC2)C1)C(C)C] |
| 122 | 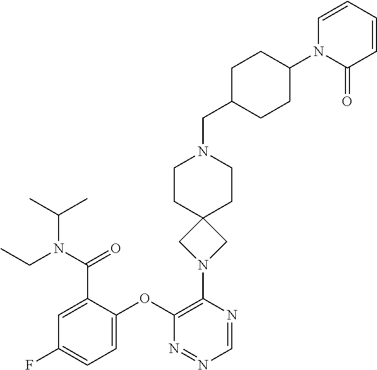 [SMILES: CCN(C(=O)c1cc(F)ccc1Oc1nncnc1N1CC2(CCN(CC3CCC(n4ccccc4=O)CC3)CC2)C1)C(C)C] |
| 123 | 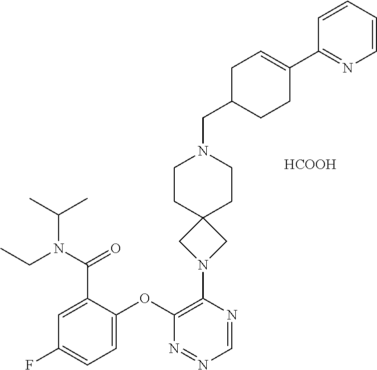 [SMILES: CCN(C(=O)c1cc(F)ccc1Oc1nncnc1N1CC2(CCN(CC3CC=C(c4ccccn4)CC3)CC2)C1)C(C)C ; "HCOOH"] |
| 124 | 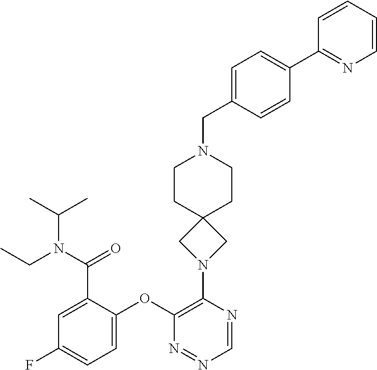 [SMILES: CCN(C(=O)c1cc(F)ccc1Oc1nncnc1N1CC2(CCN(Cc3ccc(-c4ccccn4)cc3)CC2)C1)C(C)C] |
| 125 | 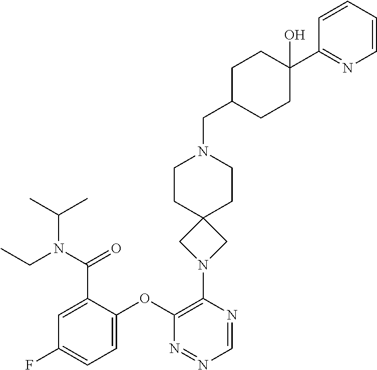 [SMILES: CCN(C(=O)c1cc(F)ccc1Oc1nncnc1N1CC2(CCN(CC3CCC(O)(c4ccccn4)CC3)CC2)C1)C(C)C] |
| 126 | 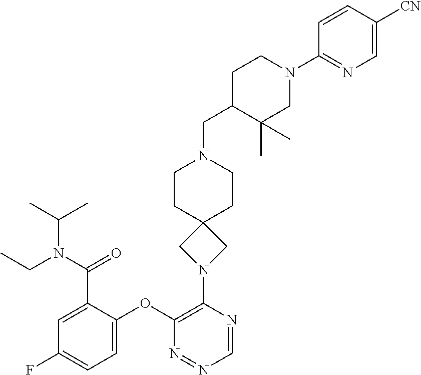 [SMILES: CCN(C(=O)c1cc(F)ccc1Oc1nncnc1N1CC2(CCN(CC3CCN(c4ccc(C#N)cn4)CC3(C)C)CC2)C1)C(C)C] |
| 127 | 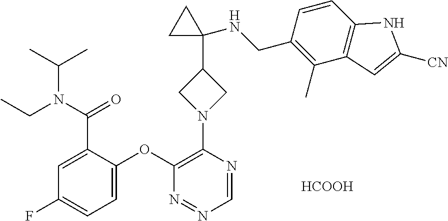 [SMILES: CCN(C(=O)c1cc(F)ccc1Oc1nncnc1N1CC(C2(NCc3ccc4[nH]c(C#N)cc4c3C)CC2)C1)C(C)C ; "HCOOH"] |
| 128 | 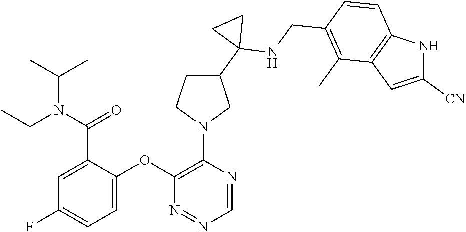 [SMILES: CCN(C(=O)c1cc(F)ccc1Oc1nncnc1N1CCC(C2(NCc3ccc4[nH]c(C#N)cc4c3C)CC2)C1)C(C)C] |
| 129 | 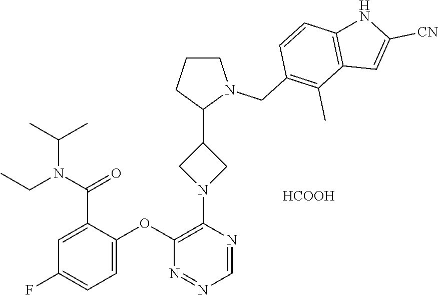 [SMILES: CCN(C(=O)c1cc(F)ccc1Oc1nncnc1N1CC(C2CCCN2Cc2ccc3[nH]c(C#N)cc3c2C)C1)C(C)C ; "HCOOH"] |
| 130 | 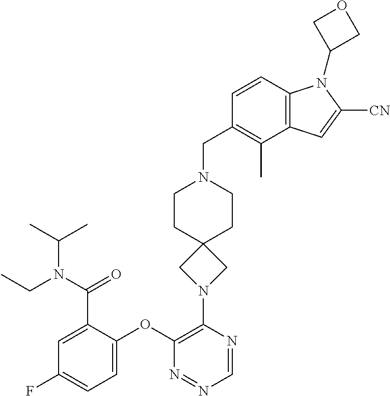 [SMILES: CCN(C(=O)c1cc(F)ccc1Oc1nncnc1N1CC2(CCN(Cc3ccc4c(cc(C#N)n4C4COC4)c3C)CC2)C1)C(C)C] |
| 131 | 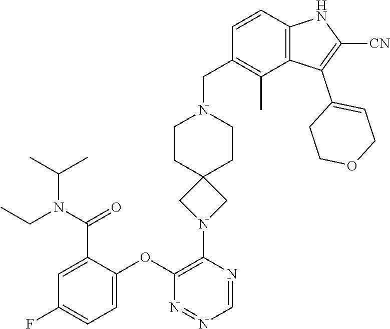 [SMILES: CCN(C(=O)c1cc(F)ccc1Oc1nncnc1N1CC2(CCN(Cc3ccc4[nH]c(C#N)c(C5=CCOCC5)c4c3C)CC2)C1)C(C)C] |
| 132 | 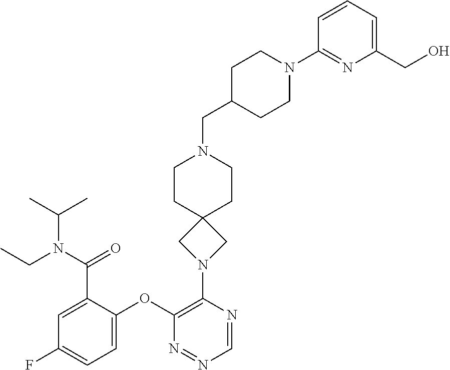 [SMILES: CCN(C(=O)c1cc(F)ccc1Oc1nncnc1N1CC2(CCN(CC3CCN(c4cccc(CO)n4)CC3)CC2)C1)C(C)C] |
| 133 | 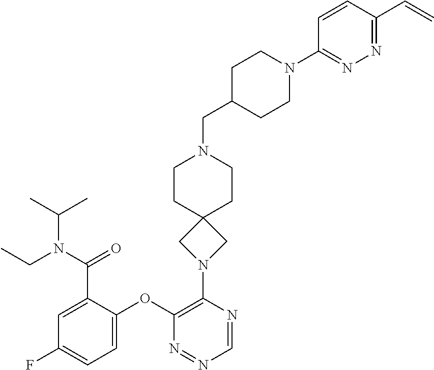 [SMILES: C=Cc1ccc(N2CCC(CN3CCC4(CC3)CN(c3ncnnc3Oc3ccc(F)cc3C(=O)N(CC)C(C)C)C4)CC2)nn1] |
| 134 | 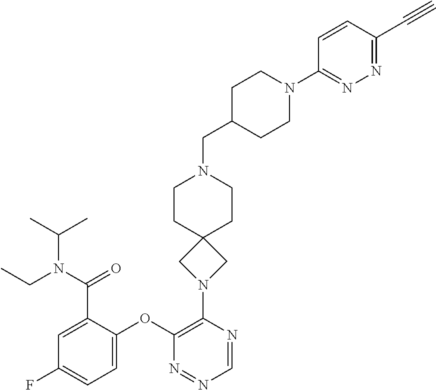 [SMILES: C#Cc1ccc(N2CCC(CN3CCC4(CC3)CN(c3ncnnc3Oc3ccc(F)cc3C(=O)N(CC)C(C)C)C4)CC2)nn1] |
| 135 | 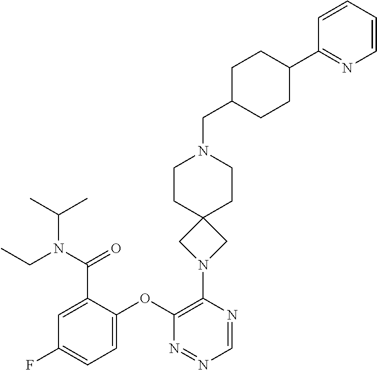 [SMILES: CCN(C(=O)c1cc(F)ccc1Oc1nncnc1N1CC2(CCN(CC3CCC(c4ccccn4)CC3)CC2)C1)C(C)C] |
| 136 | 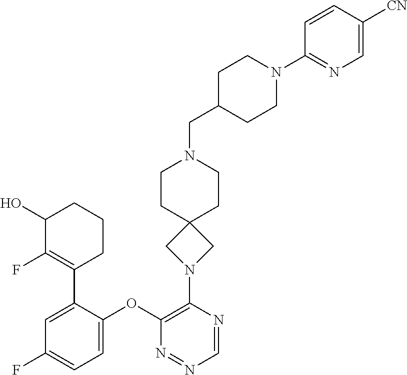 [SMILES: N#Cc1ccc(N2CCC(CN3CCC4(CC3)CN(c3ncnnc3Oc3ccc(F)cc3C3=C(F)C(O)CCC3)C4)CC2)nc1] |
| 137 | 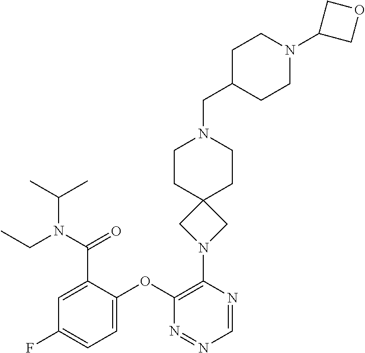 [SMILES: CCN(C(=O)c1cc(F)ccc1Oc1nncnc1N1CC2(CCN(CC3CCN(C4COC4)CC3)CC2)C1)C(C)C] |
| 138 | 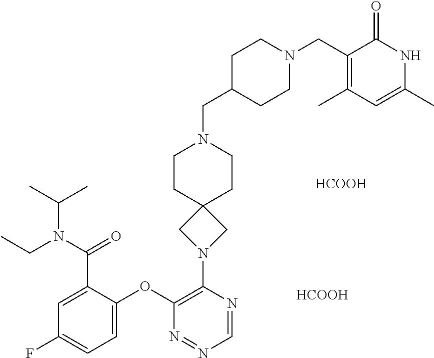 [SMILES: CCN(C(=O)c1cc(F)ccc1Oc1nncnc1N1CC2(CCN(CC3CCN(Cc4c(C)cc(C)[nH]c4=O)CC3)CC2)C1)C(C)C ; "HCOOH" ; "HCOOH"] |
| 139 | 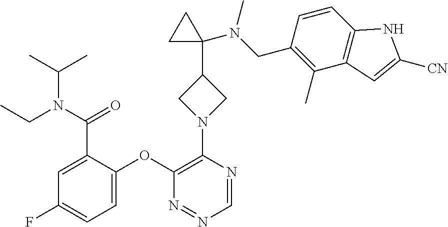 [SMILES: CCN(C(=O)c1cc(F)ccc1Oc1nncnc1N1CC(C2(N(C)Cc3ccc4[nH]c(C#N)cc4c3C)CC2)C1)C(C)C] |
| 140 | 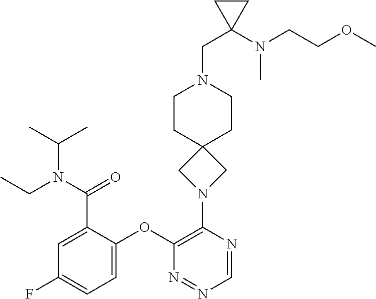 [SMILES: CCN(C(=O)c1cc(F)ccc1Oc1nncnc1N1CC2(CCN(CC3(N(C)CCOC)CC3)CC2)C1)C(C)C] |
| 141 | 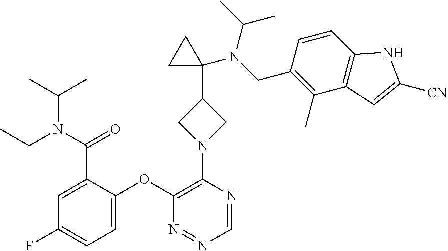 [SMILES: CCN(C(=O)c1cc(F)ccc1Oc1nncnc1N1CC(C2(N(Cc3ccc4[nH]c(C#N)cc4c3C)C(C)C)CC2)C1)C(C)C] |
| 142 | 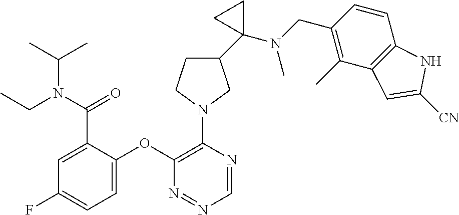 [SMILES: CCN(C(=O)c1cc(F)ccc1Oc1nncnc1N1CCC(C2(N(C)Cc3ccc4[nH]c(C#N)cc4c3C)CC2)C1)C(C)C] |
| 143 | 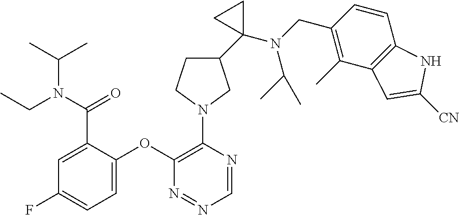 [SMILES: CCN(C(=O)c1cc(F)ccc1Oc1nncnc1N1CCC(C2(N(Cc3ccc4[nH]c(C#N)cc4c3C)C(C)C)CC2)C1)C(C)C] |
| 144 | 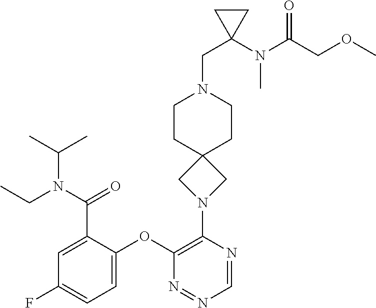 [SMILES: CCN(C(=O)c1cc(F)ccc1Oc1nncnc1N1CC2(CCN(CC3(N(C)C(=O)COC)CC3)CC2)C1)C(C)C] |
| 145 | 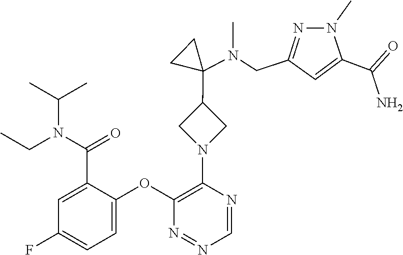 [SMILES: CCN(C(=O)c1cc(F)ccc1Oc1nncnc1N1CC(C2(N(C)Cc3cc(C(N)=O)n(C)n3)CC2)C1)C(C)C] |
| 146 | 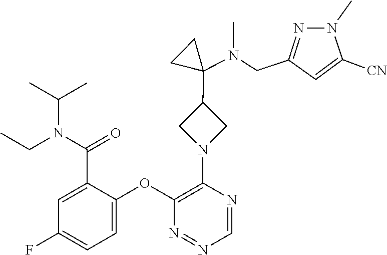 [SMILES: CCN(C(=O)c1cc(F)ccc1Oc1nncnc1N1CC(C2(N(C)Cc3cc(C#N)n(C)n3)CC2)C1)C(C)C] |
| 147 | 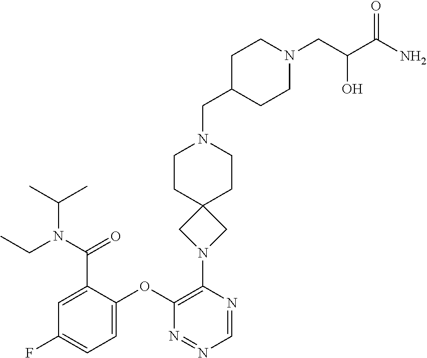 [SMILES: CCN(C(=O)c1cc(F)ccc1Oc1nncnc1N1CC2(CCN(CC3CCN(CC(O)C(N)=O)CC3)CC2)C1)C(C)C] |
| 148 | 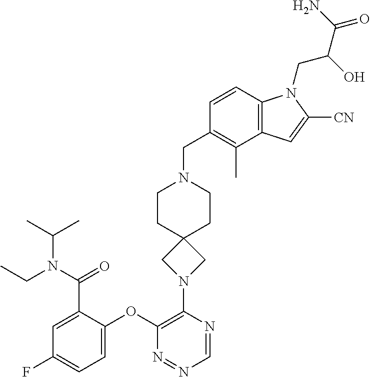 [SMILES: CCN(C(=O)c1cc(F)ccc1Oc1nncnc1N1CC2(CCN(Cc3ccc4c(cc(C#N)n4CC(O)C(N)=O)c3C)CC2)C1)C(C)C] |
| 149 | 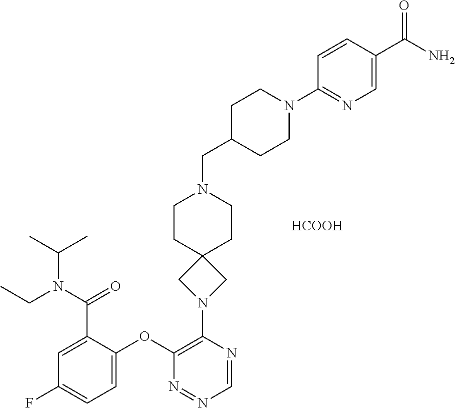 [SMILES: CCN(C(=O)c1cc(F)ccc1Oc1nncnc1N1CC2(CCN(CC3CCN(c4ccc(C(N)=O)cn4)CC3)CC2)C1)C(C)C ; "HCOOH"] |
| 150 | 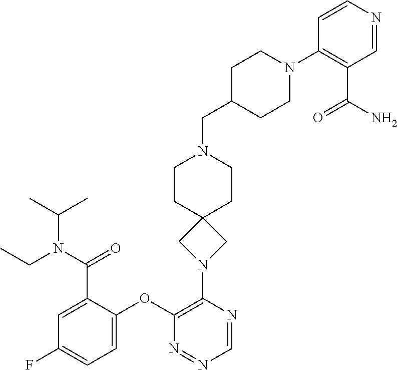 [SMILES: CCN(C(=O)c1cc(F)ccc1Oc1nncnc1N1CC2(CCN(CC3CCN(c4ccncc4C(N)=O)CC3)CC2)C1)C(C)C] |
| 151 | 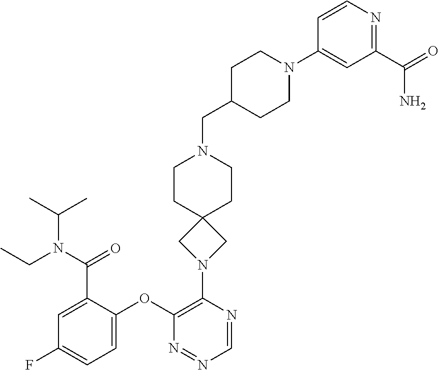 [SMILES: CCN(C(=O)c1cc(F)ccc1Oc1nncnc1N1CC2(CCN(CC3CCN(c4ccnc(C(N)=O)c4)CC3)CC2)C1)C(C)C] |
| 152 | 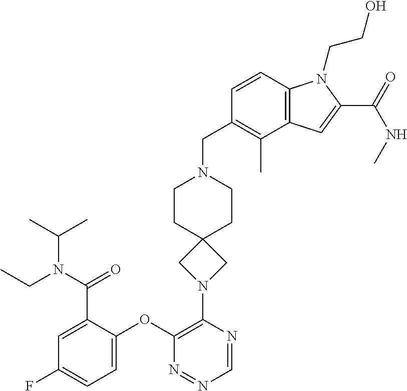 [SMILES: CCN(C(=O)c1cc(F)ccc1Oc1nncnc1N1CC2(CCN(Cc3ccc4c(cc(C(=O)NC)n4CCO)c3C)CC2)C1)C(C)C] |
| 153 | 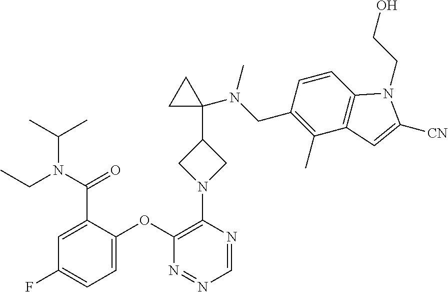 [SMILES: CCN(C(=O)c1cc(F)ccc1Oc1nncnc1N1CC(C2(N(C)Cc3ccc4c(cc(C#N)n4CCO)c3C)CC2)C1)C(C)C] |
| 154 | 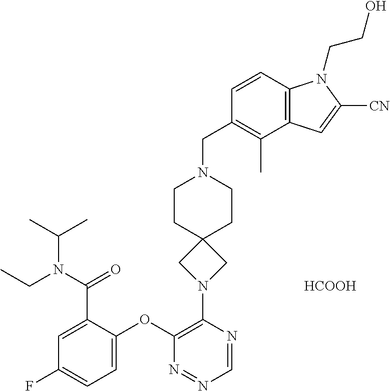 [SMILES: CCN(C(=O)c1cc(F)ccc1Oc1nncnc1N1CC2(CCN(Cc3ccc4c(cc(C#N)n4CCO)c3C)CC2)C1)C(C)C ; "HCOOH"] |
| 155 | 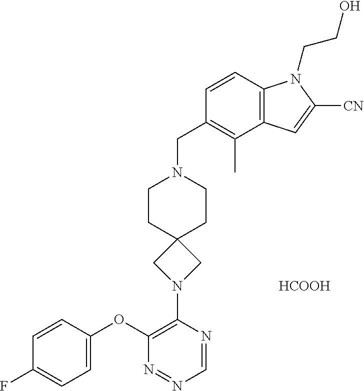 [SMILES: Cc1c(CN2CCC3(CC2)CN(c2ncnnc2Oc2ccc(F)cc2)C3)ccc2c1cc(C#N)n2CCO ; "HCOOH"] |
| 156 | 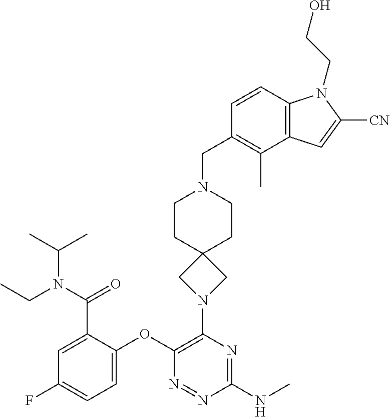 [SMILES: CCN(C(=O)c1cc(F)ccc1Oc1nnc(NC)nc1N1CC2(CCN(Cc3ccc4c(cc(C#N)n4CCO)c3C)CC2)C1)C(C)C] |
| 157 | 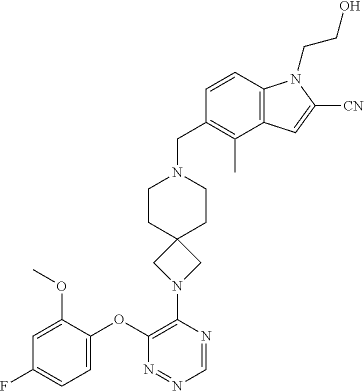 [SMILES: COc1cc(F)ccc1Oc1nncnc1N1CC2(CCN(Cc3ccc4c(cc(C#N)n4CCO)c3C)CC2)C1] |
| 158 | 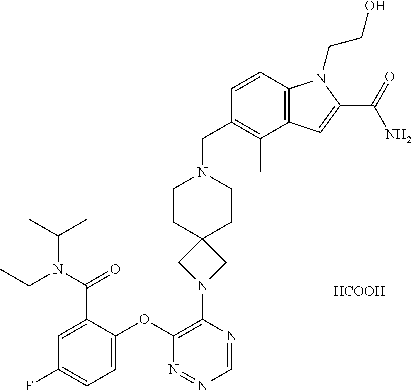 [SMILES: CCN(C(=O)c1cc(F)ccc1Oc1nncnc1N1CC2(CCN(Cc3ccc4c(cc(C(N)=O)n4CCO)c3C)CC2)C1)C(C)C ; "HCOOH"] |
| 159 | 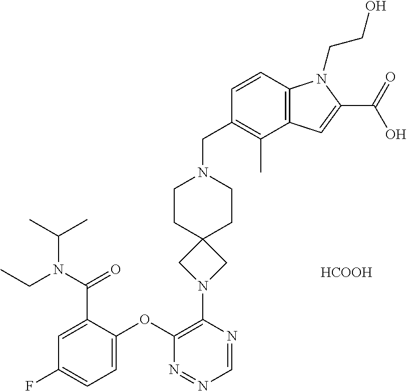 [SMILES: CCN(C(=O)c1cc(F)ccc1Oc1nncnc1N1CC2(CCN(Cc3ccc4c(cc(C(=O)O)n4CCO)c3C)CC2)C1)C(C)C ; "HCOOH"] |
| 160 | 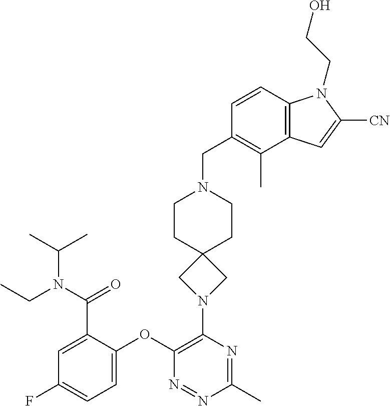 [SMILES: CCN(C(=O)c1cc(F)ccc1Oc1nnc(C)nc1N1CC2(CCN(Cc3ccc4c(cc(C#N)n4CCO)c3C)CC2)C1)C(C)C] |
| 161 | 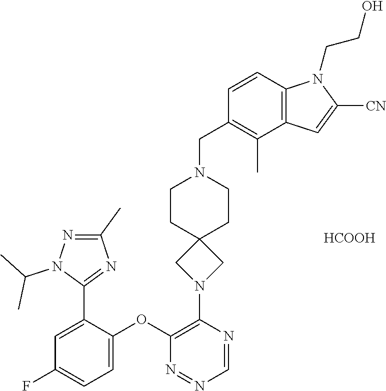 [SMILES: Cc1nc(-c2cc(F)ccc2Oc2nncnc2N2CC3(CCN(Cc4ccc5c(cc(C#N)n5CCO)c4C)CC3)C2)n(C(C)C)n1 ; "HCOOH"] |
| 162 | 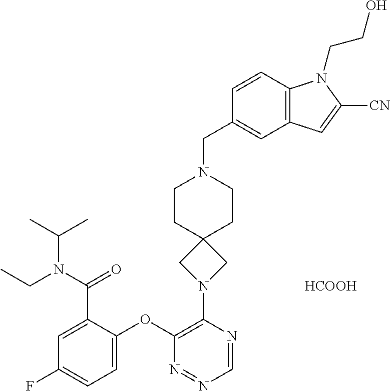 [SMILES: CCN(C(=O)c1cc(F)ccc1Oc1nncnc1N1CC2(CCN(Cc3ccc4c(c3)cc(C#N)n4CCO)CC2)C1)C(C)C ; "HCOOH"] |
| 163 | 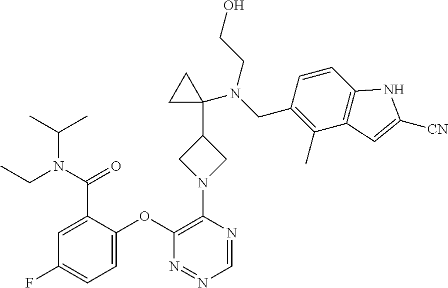 [SMILES: CCN(C(=O)c1cc(F)ccc1Oc1nncnc1N1CC(C2(N(CCO)Cc3ccc4[nH]c(C#N)cc4c3C)CC2)C1)C(C)C] |
| 164 | 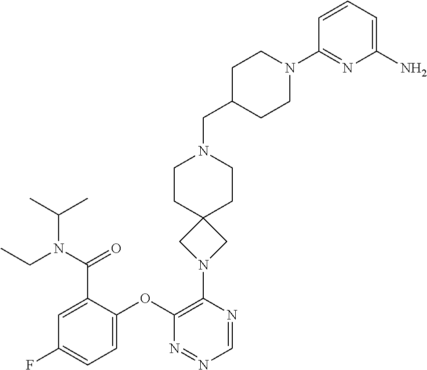 [SMILES: CCN(C(=O)c1cc(F)ccc1Oc1nncnc1N1CC2(CCN(CC3CCN(c4cccc(N)n4)CC3)CC2)C1)C(C)C] |
| 165 | 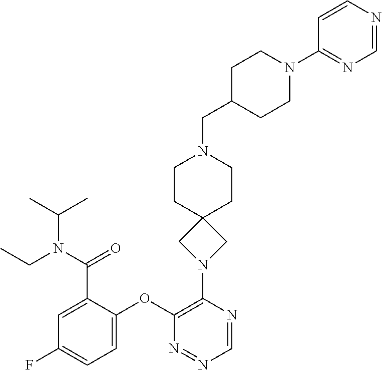 [SMILES: CCN(C(=O)c1cc(F)ccc1Oc1nncnc1N1CC2(CCN(CC3CCN(c4ccncn4)CC3)CC2)C1)C(C)C] |
| 166 | 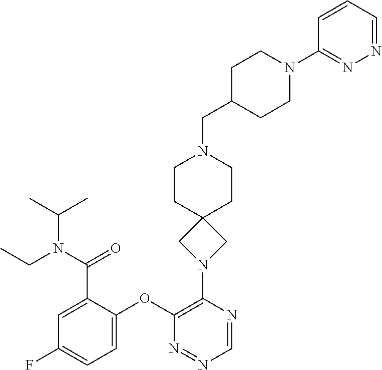 [SMILES: CCN(C(=O)c1cc(F)ccc1Oc1nncnc1N1CC2(CCN(CC3CCN(c4cccnn4)CC3)CC2)C1)C(C)C] |
| 167 | 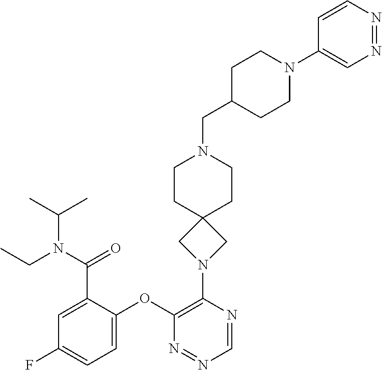 [SMILES: CCN(C(=O)c1cc(F)ccc1Oc1nncnc1N1CC2(CCN(CC3CCN(c4ccnnc4)CC3)CC2)C1)C(C)C] |
| 168 | 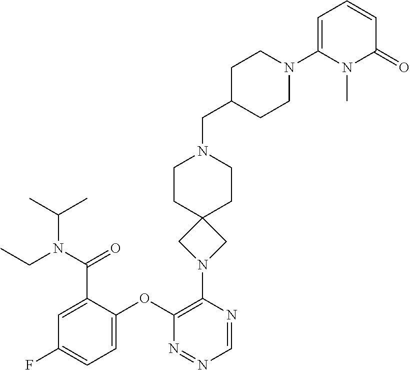 [SMILES: CCN(C(=O)c1cc(F)ccc1Oc1nncnc1N1CC2(CCN(CC3CCN(c4cccc(=O)n4C)CC3)CC2)C1)C(C)C] |
| 169 | 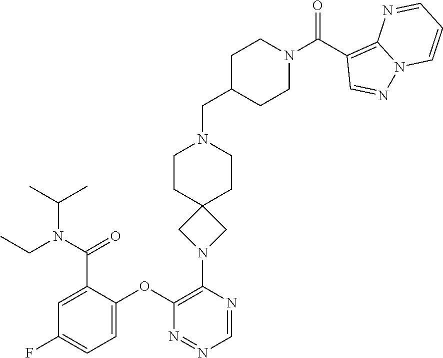 [SMILES: CCN(C(=O)c1cc(F)ccc1Oc1nncnc1N1CC2(CCN(CC3CCN(C(=O)c4cnn5cccnc45)CC3)CC2)C1)C(C)C] |
| 170 | 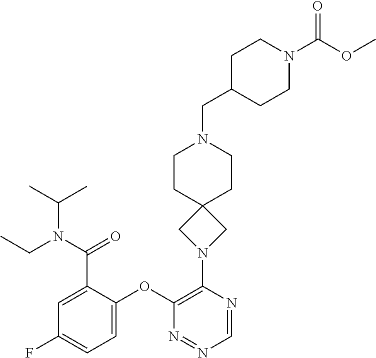 [SMILES: CCN(C(=O)c1cc(F)ccc1Oc1nncnc1N1CC2(CCN(CC3CCN(C(=O)OC)CC3)CC2)C1)C(C)C] |
| 171 | 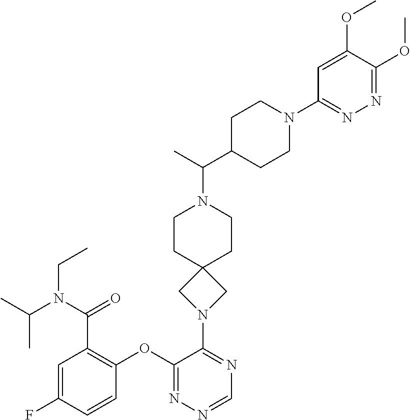 [SMILES: CCN(C(=O)c1cc(F)ccc1Oc1nncnc1N1CC2(CCN(C(C)C3CCN(c4cc(OC)c(OC)nn4)CC3)CC2)C1)C(C)C] |
| 172 | 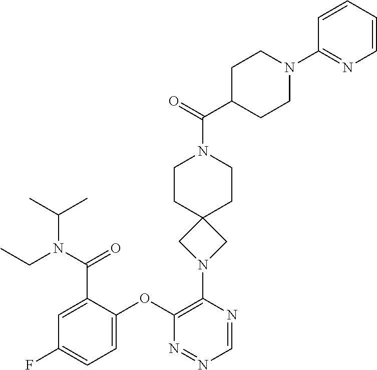 [SMILES: CCN(C(=O)c1cc(F)ccc1Oc1nncnc1N1CC2(CCN(C(=O)C3CCN(c4ccccn4)CC3)CC2)C1)C(C)C] |
| 173 | 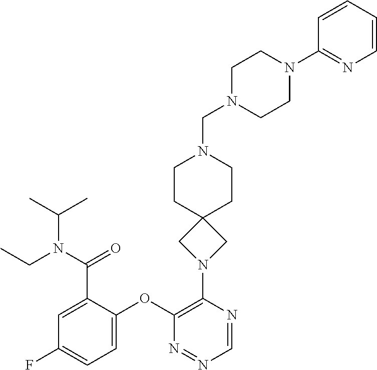 [SMILES: CCN(C(=O)c1cc(F)ccc1Oc1nncnc1N1CC2(CCN(CN3CCN(c4ccccn4)CC3)CC2)C1)C(C)C] |
| 174 | 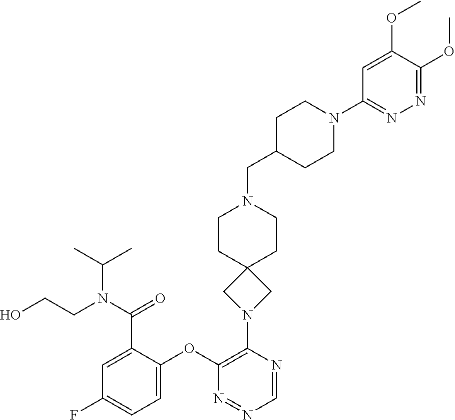 [SMILES: COc1cc(N2CCC(CN3CCC4(CC3)CN(c3ncnnc3Oc3ccc(F)cc3C(=O)N(CCO)C(C)C)C4)CC2)nnc1OC] |
| 175 | 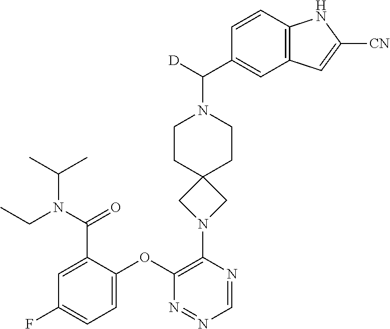 [SMILES: [2H]C(c1ccc2[nH]c(C#N)cc2c1)N1CCC2(CC1)CN(c1ncnnc1Oc1ccc(F)cc1C(=O)N(CC)C(C)C)C2] |
| 176 | 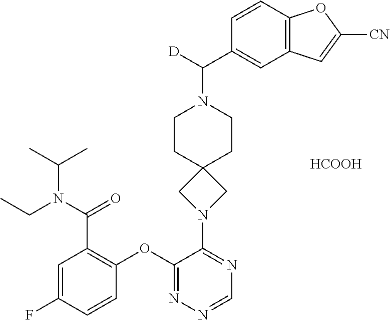 [SMILES: [2H]C(c1ccc2oc(C#N)cc2c1)N1CCC2(CC1)CN(c1ncnnc1Oc1ccc(F)cc1C(=O)N(CC)C(C)C)C2 ; "HCOOH"] |
| 177 | 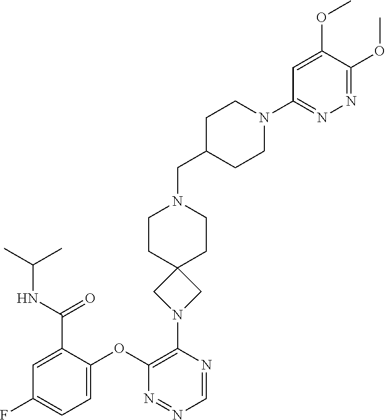 [SMILES: COc1cc(N2CCC(CN3CCC4(CC3)CN(c3ncnnc3Oc3ccc(F)cc3C(=O)NC(C)C)C4)CC2)nnc1OC] |
| 178 | 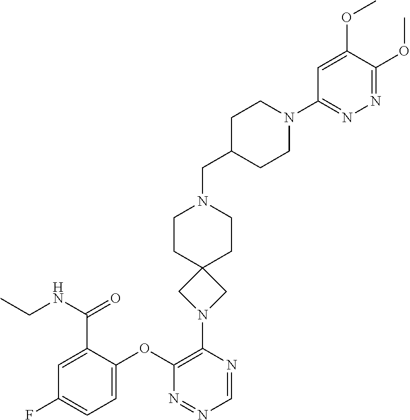 [SMILES: CCNC(=O)c1cc(F)ccc1Oc1nncnc1N1CC2(CCN(CC3CCN(c4cc(OC)c(OC)nn4)CC3)CC2)C1] |
| 179 | 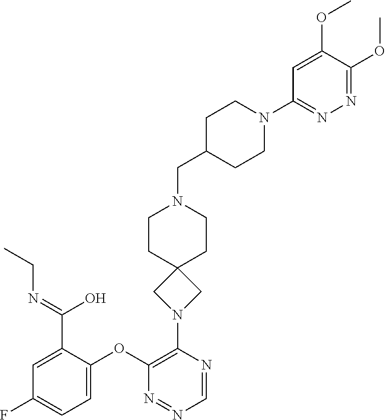 [SMILES: CC/N=C(\O)c1cc(F)ccc1Oc1nncnc1N1CC2(CCN(CC3CCN(c4cc(OC)c(OC)nn4)CC3)CC2)C1] |
| 180 | 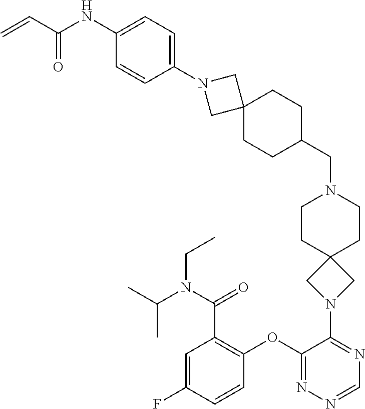 [SMILES: C=CC(=O)Nc1ccc(N2CC3(CCC(CN4CCC5(CC4)CN(c4ncnnc4Oc4ccc(F)cc4C(=O)N(CC)C(C)C)C5)CC3)C2)cc1] |
| 181 | 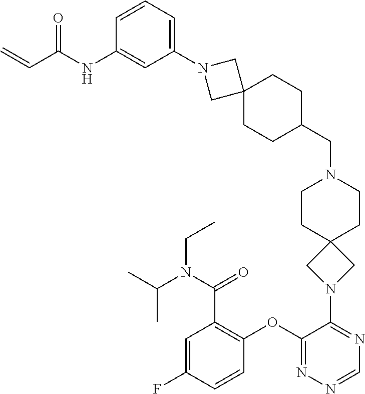 [SMILES: C=CC(=O)Nc1cccc(N2CC3(CCC(CN4CCC5(CC4)CN(c4ncnnc4Oc4ccc(F)cc4C(=O)N(CC)C(C)C)C5)CC3)C2)c1] |
| 182 | 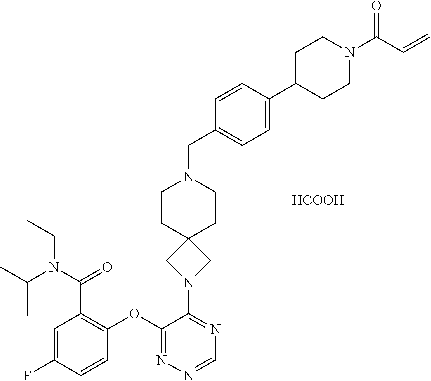 [SMILES: C=CC(=O)N1CCC(c2ccc(CN3CCC4(CC3)CN(c3ncnnc3Oc3ccc(F)cc3C(=O)N(CC)C(C)C)C4)cc2)CC1 ; "HCOOH"] |
| 183 | 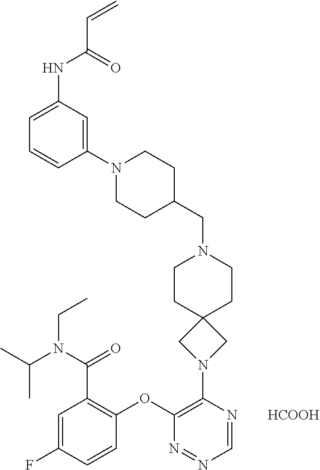 [SMILES: C=CC(=O)Nc1cccc(N2CCC(CN3CCC4(CC3)CN(c3ncnnc3Oc3ccc(F)cc3C(=O)N(CC)C(C)C)C4)CC2)c1 ; "HCOOH"] |
| 184 | 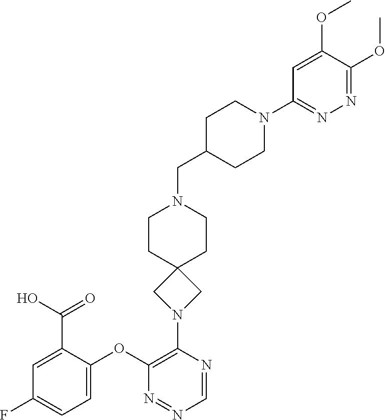 [SMILES: COc1cc(N2CCC(CN3CCC4(CC3)CN(c3ncnnc3Oc3ccc(F)cc3C(=O)O)C4)CC2)nnc1OC] |

19. A pharmaceutical composition, comprising the compound and/or the pharmaceutically acceptable salt thereof according to any one of claims 1-18, and optionally comprising a pharmaceutically acceptable excipient.

20. A method of in vivo or in vitro inhibiting menin-MLL interaction, comprising contacting menin with an effective amount of the compound and/or the pharmaceutically acceptable salt thereof according to any one of claims 1-18.

21. Use of the compound and/or the pharmaceutically acceptable salt thereof according to any one of claims 1-18 in the manufacture of a medicament for treating or preventing a disease mediated by menin-MLL interaction or at least in part by menin-MLL interaction, wherein the disease mediated by menin-MLL interaction or at least in part by menin-MLL interaction is preferably cancer; the cancer is preferably a hematologic malignancy or solid tumor, including leukemia, lymphoma and myeloma; and the cancer is more preferably selected from acute leukemia, chronic leukemia, myeloid leukemia, myelogenous leukemia, lymphocytic leukemia, lymphoblastic leukemia, acute myelogenous leukemia (AML), chronic myelogenous leukemia (CML), acute lymphocytic leukemia (ALL), B-cell acute lymphocytic leukemia (B-ALL), T-cell prolymphocytic leukemia (T-PLL), chronic lymphocytic leukemia (CLL), chronic myelocytic leukemia, large granular lymphocytic leukemia, hairy cell leukemia (HCL), mixed lineage leukemia (MLL), MLL-related leukemia, MLL-rearranged leukemia (MLL-r), MLL-PTD leukemia, MLL-positive leukemia, NPM1 mutant leukemia, leukemia exhibiting HOX/MEIS1 gene expression signatures, myelodysplastic syndrome (MDS), myeloproliferative neoplasm (MPN), multiple myeloma (MM), Hodgkin's lymphoma, non-Hodgkin's lymphoma, diffuse large B-cell lymphoma (DLBCL), B-cell lymphoma, T-cell lymphoma, mantle cell lymphoma, follicular lymphoma (FL), Waldenstrom's macroglobulinemia, prostate cancer, breast cancer, lung cancer, liver cancer, colon cancer, colorectal cancer, pancreatic cancer, melanoma, and glioblastoma (GBM).

22. A method of treating or preventing a disease in a subject, comprising administering to the subject in need thereof an effective amount of the compound and/or the pharmaceutically acceptable salt thereof according to any one of claims 1-18, wherein the disease is a disease mediated by menin-MLL interaction or at least in part by menin-MLL interaction; the disease is preferably cancer; the cancer is preferably a hematologic malignancy or solid tumor, including leukemia, lymphoma and myeloma; and the cancer is more preferably selected from acute leukemia, chronic leukemia, myeloid leukemia, myelogenous leukemia, lymphocytic leukemia, lymphoblastic leukemia, acute myelogenous leukemia (AML), chronic myelogenous leukemia (CML), acute lymphocytic leukemia (ALL), B-cell acute lymphocytic leukemia (B-ALL), T-cell prolymphocytic leukemia (T-PLL), chronic lymphocytic leukemia (CLL), chronic myelocytic leukemia, large granular lymphocytic leukemia, hairy cell leukemia (HCL), mixed lineage leukemia (MLL), MLL-related leukemia, MLL-rearranged leukemia (MLL-r), MLL-PTD leukemia, MLL-positive leukemia, NPM1 mutant leukemia, leukemia exhibiting HOX/MEIS1 gene expression signatures, myelodysplastic syndrome (MDS), myeloproliferative neoplasm (MPN), multiple myeloma (MM), Hodgkin's lymphoma, non-Hodgkin's lymphoma, diffuse large B-cell lymphoma (DLBCL), B-cell lymphoma, T-cell lymphoma, mantle cell lymphoma, follicular lymphoma (FL), Waldenstrom's macroglobulinemia, prostate cancer, breast cancer, lung cancer, liver cancer, colon cancer, colorectal cancer, pancreatic cancer, melanoma, and glioblastoma (GBM).

23. The compound and/or the pharmaceutically acceptable salt thereof according to any one of claims 1-18, for use as a medicament.

24. The compound and/or the pharmaceutically acceptable salt thereof according to any one of claims 1-18, for use in treating or preventing a disease mediated by menin-MLL interaction or at least in part by menin-MLL interaction, wherein the disease is preferably cancer; the cancer is preferably a hematologic malignancy or solid tumor, including leukemia, lymphoma and myeloma; and the cancer is more preferably selected from acute leukemia, chronic leukemia, myeloid leukemia, myelogenous leukemia, lymphocytic leukemia, lymphoblastic leukemia, acute myelogenous leukemia (AML), chronic myelogenous leukemia (CML), acute lymphocytic leukemia (ALL), B-cell acute lymphocytic leukemia (B-ALL), T-cell prolymphocytic leukemia (T-PLL), chronic lymphocytic leukemia (CLL), chronic myelocytic leukemia, large granular lymphocytic leukemia, hairy cell leukemia (HCL), mixed lineage leukemia (MLL), MLL-related leukemia, MLL-rearranged leukemia (MLL-r), MLL-PTD leukemia, MLL-positive leukemia, NPM1 mutant leukemia, leukemia exhibiting HOX/MEIS1 gene expression signatures, myelodysplastic syndrome (MDS), myeloproliferative neoplasm (MPN), multiple myeloma (MM), Hodgkin's lymphoma, non-Hodgkin's lymphoma, diffuse large B-cell lymphoma (DLBCL), B-cell lymphoma, T-cell lymphoma, mantle cell lymphoma, follicular lymphoma (FL), Waldenstrom's macroglobulinemia, prostate cancer, breast cancer, lung cancer, liver cancer, colon cancer, colorectal cancer, pancreatic cancer, melanoma, and glioblastoma (GBM).

25. A pharmaceutical combination, comprising the compound and/or the pharmaceutically acceptable salt thereof according to any one of claims 1-18, and at least one additional therapeutic agent, wherein the additional therapeutic agent is preferably selected from an anti-neoplastic active agent, an anti-inflammatory agent or an immunomodulator, wherein the anti-neoplastic active agent includes a chemotherapeutic agent, an immune checkpoint inhibitor or agonist, and a targeted therapeutic agent.

26. A compound of formula (III): 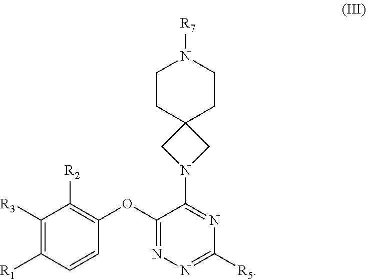
or a pharmaceutically acceptable salt, a solvate, a racemic mixture, an enantiomer, a diastereomer or a tautomer thereof, or a deuterate thereof, wherein
R₁, R₂, R₃ and R₅ are as defined in any one of claims 1-17;
R₇ is hydrogen or an amino protecting group; preferably, R₇ is hydrogen or an amino protecting group selected from Boc (tert-butoxycarbonyl), benzyl, Pmb (p-methoxybenzyl) and Cbz (benzyloxycarbonyl); and more preferably, R₇ is hydrogen or Boc (tert-butoxycarbonyl);
provided that, when R₅ is H or Cl, R₂ is not —C(O)N(CH(CH₃)₂)₂, —C(O)N(CH(CH₃)₂)(CH₃), —C(O)N(CH(CH₃)₂)(CH₂CH₃), —C(O)N(CH(CH₃)₂)(CH₂CHF₂), —C(O)N(CH(CH₃)₂)(CH₂CF₃), —C(O)N(CH(CH₃)₂)(CH₂CH₂OH), —C(O)N(cyclopropyl)₂, —C(O)N(CH(CH₃)₂)(cyclopropyl), —C(O)N(CH₂CH₃)(cyclopropyl), —C(O)N(CH(CH₃)₂)(cyclobutyl), pyrimidyl substituted with isopropyl, and pyrazolyl substituted with isopropyl and/or Cl.

27. A compound selected from: 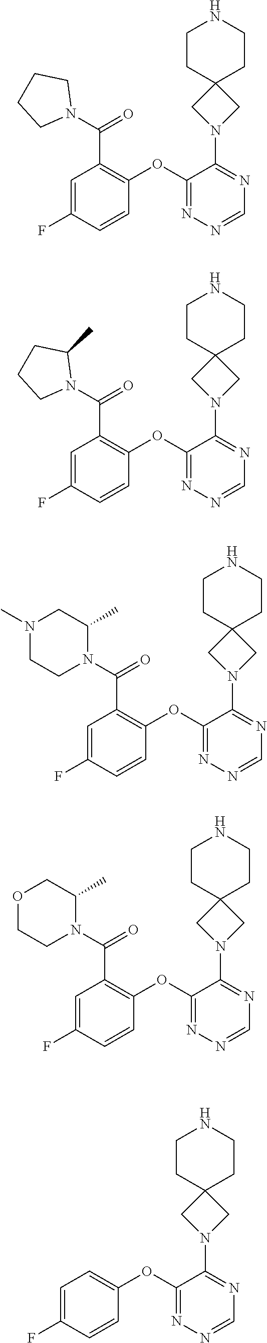 [SMILES: O=C(c1cc(F)ccc1Oc1nncnc1N1CC2(CCNCC2)C1)N1CCCC1 ; C[C@@H]1CCCN1C(=O)c1cc(F)ccc1Oc1nncnc1N1CC2(CCNCC2)C1 ; C[C@H]1CN(C)CCN1C(=O)c1cc(F)ccc1Oc1nncnc1N1CC2(CCNCC2)C1 ; C[C@H]1COCCN1C(=O)c1cc(F)ccc1Oc1nncnc1N1CC2(CCNCC2)C1 ; Fc1ccc(Oc2nncnc2N2CC3(CCNCC3)C2)cc1] 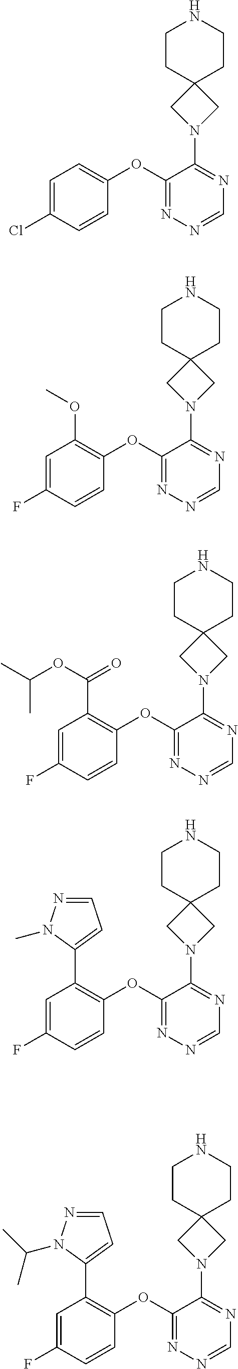 [SMILES: Clc1ccc(Oc2nncnc2N2CC3(CCNCC3)C2)cc1 ; COc1cc(F)ccc1Oc1nncnc1N1CC2(CCNCC2)C1 ; CC(C)OC(=O)c1cc(F)ccc1Oc1nncnc1N1CC2(CCNCC2)C1 ; Cn1nccc1-c1cc(F)ccc1Oc1nncnc1N1CC2(CCNCC2)C1 ; CC(C)n1nccc1-c1cc(F)ccc1Oc1nncnc1N1CC2(CCNCC2)C1] 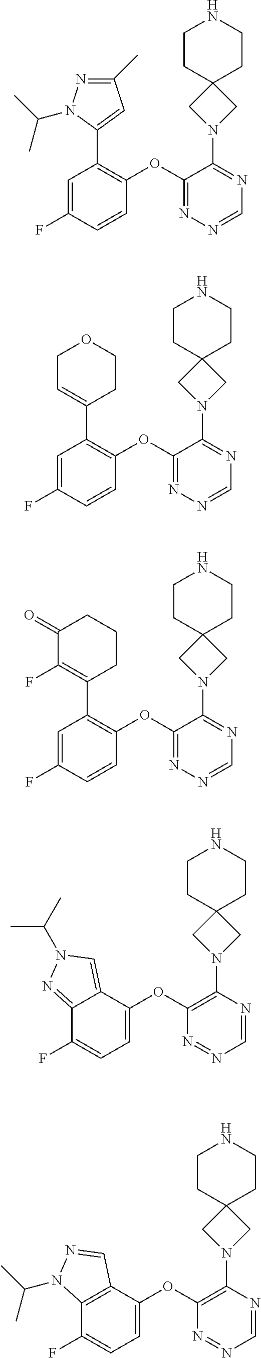 [SMILES: Cc1cc(-c2cc(F)ccc2Oc2nncnc2N2CC3(CCNCC3)C2)n(C(C)C)n1 ; Fc1ccc(Oc2nncnc2N2CC3(CCNCC3)C2)c(C2=CCOCC2)c1 ; O=C1CCCC(c2cc(F)ccc2Oc2nncnc2N2CC3(CCNCC3)C2)=C1F ; CC(C)n1cc2c(Oc3nncnc3N3CC4(CCNCC4)C3)ccc(F)c2n1 ; CC(C)n1ncc2c(Oc3nncnc3N3CC4(CCNCC4)C3)ccc(F)c21] 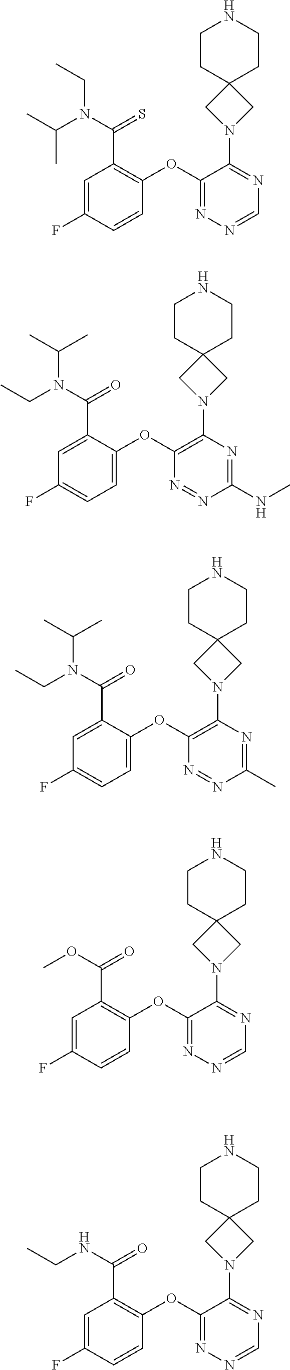
or a pharmaceutically acceptable salt, a solvate, a racemic mixture, an enantiomer, a diastereomer or a tautomer thereof, or a deuterate thereof.
